SEQUENCE LISTING (184 sequences)

SEQ ID NO: 1 - protein, 752 aa, Chlamydomonas reinhardtii
MDKAERAAGGPNAASEDDWLLEFWPEPAADFPAPVAPMLSQHQDAAQLPEAMPQQQGLALGGYGLTQQPSDFMQTGMPGFDAFSSGKAATLGLPLLADPQRASTDGASALMNAAQQSSEYMLAPGMGGMPHLLAPSVGTALPGTGHTGFADLSMGGMAGGIPGLGGPGIMHGQYFMQPQRAATGPAKSRLRWTPELHNRFVNAVNSLGGPDKATPKGILKLMGVDGLTIYHIKSHLQKYRLNIRLPGESGLAGDSADGSDGERSDGEGGVRRATSLERADTMSGMAGGAAAALGRAGGTPGGALISPGLAGGTSSTGGMAAGGGGGGGLVTEPSISRGTVLNAAGAVATAAPAAAAPAGGSAAVKRPAGTSLSSGSTASATRRNLEEALLFQMELQKKLHEQLETQRQLQLSLEAHGRYIASLMEQEGLTSRLPELSGGAPAAAPVAAGGAAGGMIAPPPPQQQLQHQPQLLQPQGSLPAGGSSEAHAAAGAGTMVVHQQQQQHVHHHHQQQQVQMQQHARHCDTCGAGGAGGAPSGGSSMQQLQAAEQQRTELVVAGRLGSMPAPASSSPLAGQAHQQQPLAGGAAHLVHVHSHTPGGQPHVQHQDAFAGAATAAAHASPGLPQSHSHLLPADLSSNAGPDTSAGQIKPEPDMSQQQQQQEQQEAEQLAQGLLNDSSAGAGAVSGSDGGGLGDFDFGDFGDLDGGAQGGLLGPGDLIGIAELEAAAAHEQQQEQEHDPLDADRAKRQRVEP

SEQ ID NO: 2 - DNA, 2259 bp, Chlamydomonas reinhardtii
  source 1..2259: mol_type = other DNA
atggacaaagctgaacgcgctgctggtggccctaacgctgcaagcgaggacgactggctgctggagttttggccggagcctgcagcggactttcctgcaccggtcgctccgatgctgtcgcagcatcaagacgcagcacagctgcctgaggccatgccgcagcagcaaggactggcgctgggtggatatggtctcacgcagcagccttctgactttatgcaaacgggcatgcccggcttcgacgcgttcagcagcgggaaggctgcaaccctcgggctgcccctgcttgccgacccccagcgcgcctccaccgacggcgcctctgcgcttatgaacgcggcgcagcagtcctcagagtacatgctggcccccggcatgggcggcatgccgcatctactagcaccgagcgttggcacggcgctgcccggcactgggcacaccggcttcgcggacctgtccatggggggcatggcggggggcatcccgggcctcggggggccaggcattatgcatgggcagtacttcatgcagccgcagcgagcagccacgggccccgccaagagccggctgcgctggacgccggagctgcacaaccgcttcgtcaacgcggtgaactcgctgggggggccggacaaggccacgcccaagggcatccttaagctcatgggcgtcgacggcctcaccatctaccacatcaagtcgcacctgcagaagtaccgcctcaacatccggctgccgggagagagcggcctcgcgggcgactcggcggacggctcggacggcgagcgctcggacggcgagggcggcgtgcggcgcgccacctcgctggagcgggcagacaccatgtcggggatggcgggaggggccgccgcagcgttagggagagcgggcgggacgccgggcggtgcgctaatctcccccggccttgccggcgggacgtcaagcaccggtgggatggcagccggcggcggcgggggtggcggcttggtgactgagcccagcatctctaggggcacggtcctcaacgcggccggcgcagttgccaccgccgcgccggctgcggcggcgcctgccggcgggtccgccgccgtgaagcggccggcgggtacgtctctgagcagcggcagcactgcctcggctactcggcgcaatctggaggaggcgctgctgttccaaatggagctgcagaagaagctgcacgagcagctggagacgcagcgtcaactgcagctgagcttggaggcgcacgggcgctacatcgccagcctcatggagcaggagggactcacctcgcgactgcccgagctcagcggcggcgcgccggcggcggcgcctgtggccgcaggcggcgcagcgggcggcatgattgcgccgccgccaccgcagcagcagctgcagcaccagccgcagctgctgcagccgcagggcagcttgccagccggcggttcctctgaagcccatgccgcagccggcgccggcacgatggtggtgcaccagcagcagcagcagcacgtgcaccatcatcaccagcagcagcaggtgcagatgcagcagcatgcccgccactgcgacacgtgtggcgccggtggcgctgggggtgcgcccagcggcggcagcagcatgcagcagcttcaggctgcggagcagcagcgcacggagcttgttgtggcggggcggctaggctccatgccggcgcccgcctcttcgtcgccgctagcagggcaggcacaccagcagcagccgctggccggcggggcggcgcacttggtgcacgtgcactcgcacacgcctggggggcagccgcacgtgcagcaccaggacgcgtttgccggcgcggctacggcggcagcgcacgcttcgccggggctgccgcagtcacattcgcacctgctcccagccgacctctccagcaacgccggtcctgacacaagcgcggggcagattaagcctgagcctgatatgtcgcagcaacagcagcaacaagagcaacaggaggcggagcagcttgcgcagggtttgctcaatgacagcagcgctggcgcgggggctgtcagcggcagcgatggtgggggccttggggactttgacttcggtgatttcggggacctggacgggggagcccagggcggcctactaggccccggagacctcattggcatcgccgagctggaggcagcggccgcgcacgagcagcagcaagagcaagagcacgacccactagatgcggatcgcgcaaagcggcagcgagtggagccatag

SEQ ID NO: 3 - protein, 804 aa, Chlamydomonas reinhardtii
MKFTHQLKFNSVPEWREHYIQYGHLKKYIYALAKKEADLQAGGQDEEALLAPLLEAERDQGPTEEGFQRELDAQLAATLSFFAVKEADLLAKVSALELDIQSLEKIPNRAEASTLARMGGSASPGGPMSSPRAAAAAAMSAMASLVSHSPSTLDLARMVNSTPPEDHRKIRVKFWENPPRHLFSTNLNTRRAKLQARFQDLYISLHDLREFLHINKEGFRKIIKKHDKLTRAVDLRARWWPNVEAHLAPAAKQAELDGAIGALTDHYAVLYTRGDVAQAEEQLSRGLREHITVERNTVWRDMAAMERKYAAVSVKQAAAPGARVTWLRTHARWLKLALSVAVFVVLANVEVWPGAENEPRNNCLALLVFASLLWSLEAVPLFVTSMALPLLIVALGVLVDRSKDPPQRMTPQQAAPAIFHAMFSQTIMLLLGGFAIAAALSKHAIAKQVAVSILSRVGRKPRNVLLAAMFTATFASMWISNVAAPVLCFGLIQPILRTLDPGHPFAKALVMGIALASNVGGMTSPISSPQNIFAIERMSLDGRPPSWLAWFAVALPVAVACNFVCWGLLLLCYQPGKAIAEVRPIKPNTDPINGTQVYIIVVSLLTVAAWCANTFLQRYTGEMGVIAVVPLVAFFGFDVLNKDDFNSFLWNVVMLAMGGLSLGEAVKSSGLLAALALTISDLVMGLSLWQVAAIFCGMVLVATTFISHTVGAMVILPIVQSVGEAMAGTPHPKLLVMAAALMCSGAMGLPVSGFPNMNAVSLEDSTGNAIVGTGDFLAVGVPSSVFAYGIIVSLGYVLMLAVGF

SEQ ID NO: 4 - DNA, 2415 bp, Chlamydomonas reinhardtii
  source 1..2415: mol_type = other DNA
atgaagtttacgcaccagctgaagtttaatagtgtgccggaatggagagaacactacatacagtatggacatcttaagaagtacatttatgcgctagctaagaaggaagcggaccttcaagctggcggccaagatgaggaggcgctgctcgccccgctgttggaagcggagcgtgatcagggccccacggaggagggcttccagcgggagttagacgcgcagcttgcggccacgctaagcttcttcgcggtgaaggaggcggacctgctcgccaaggtgtccgcactggagctggacattcaaagcctggagaagatccccaaccgcgccgaggcgtccacactggcgcgcatgggcggcagcgccagccccggcggccccatgagcagcccgcgcgccgccgccgccgccgccatgtcggccatggcctcgctggtcagccacagcccctccacactggacctggcgcgcatggtcaacagcacgccgccagaggaccaccgcaagatccgggtcaagttctgggagaacccgccccgccacctgttcagcaccaacctcaacacgcgcagggccaagctgcaggcgcgcttccaggacctgtacatctcgctgcacgacctgcgcgagttcttgcacatcaacaaggagggcttccgcaagatcatcaagaagcacgacaagctgacccgcgccgtggacctgcgcgcccgctggtggcccaacgtggaggcgcacctggcgcccgccgccaagcaggccgagctggacggcgccatcggggcgctgaccgaccactacgcagtgctgtacacgcgcggtgacgtggctcaggcggaggagcagctgtcgcgggggctgcgcgagcacatcaccgtggagcgaaacaccgtgtggcgagacatggcggccatggagcgcaagtacgcggcggtgtcggtgaagcaggcggccgcgcccggggcgcgcgtcacgtggctgcgcacgcacgcgcgctggctgaagctggccctgagtgtggcggtgttcgtggtgctggccaatgtagaggtgtggccgggcgccgagaacgagccgcgcaacaactgcctggcgctgctagtgttcgcgtcgctgctgtggagcctggaggctgtgccgctgttcgtgaccagcatggcgctgccgctgctgattgtggcgctgggggtgctggtggaccgctccaaggacccgccgcagcgcatgaccccgcagcaggcggcgccggccatcttccacgccatgttctcgcagaccatcatgctgctgctgggcggcttcgccatcgccgccgccctgtccaaacacgccatcgccaagcaggtggcggtgtccatcctgtcccgtgtgggccgcaagccgcgcaatgtgctgctggcggccatgttcacagccaccttcgccagcatgtggatcagcaacgtggcggcgcccgtgctgtgcttcggactcatacagcccatcctcaggacgctggaccccggccacccgttcgccaaggcgctggtgatgggcattgcgctggcgtccaacgtgggcggcatgaccagccccatcagcagcccgcagaacatcttcgccatcgagcgcatgagcctggacggccgcccgccctcgtggctggcctggttcgcggtggcgctgcccgtggcggtcgcatgtaactttgtgtgctggggtctgctgctgctgtgctaccagcccggcaaggccatcgccgaggtgcggcccatcaagcccaacaccgaccccatcaatggcacacaggtgtacatcattgttgtgtcgctgctgacggtggcggcctggtgcgccaacaccttcctgcagcgctacactggcgagatgggcgtgatcgcggtggtgccgctggtggcgttcttcggcttcgacgtgctcaacaaggacgacttcaacagcttcctgtggaacgtggtcatgctggccatggggggactcagcctgggcgaggccgtcaagagcagcggcctgctggcggcgctggcgctcaccatcagcgacctggtcatggggctcagcctgtggcaggtggcggccatattctgtggcatggtacttgtggccaccaccttcatcagccacacggtgggcgccatggtcatcctgcccatcgtgcagagcgtgggcgaggccatggccggcacgccgcaccccaagctgctggtcatggcggcggcgctcatgtgctcgggcgccatgggcctgcctgtgagcggcttccccaacatgaacgccgtgagcctggaggacagcaccggcaacgccatcgtgggcaccggcgacttcctggcggtgggcgtgcccagctccgtgttcgcgtacggcatcattgtctcgctgggctacgtgctcatgctggcggtgggcttctag

SEQ ID NO: 5 - protein, 359 aa, Monoraphidium sp.
  source 1..359: note = Monoraphidium neglectum
MQQDLLGGPPGPMHHDEQHEMLQQQPQQQQAQPQAQQQQQQQHPQQQHQQHQHLPGKAAFPPGMGVPGMDHFHGTPYGMQAVPMQPGHFEHLLNAMPVTGHSLSSSFATDNVHMSGAQPTLYLAAGFNMAGSKPGVSAAGSAGGGGGGGSKTRLRWTPELHSSFVRSVQQLGGPDKATPKGILKAMNMDGLTIFHIKSHLQKYRLNARVPGASSVDGGSDGCAAGDSAEGNSGSRPASAALDGLGSVPVSALTRKNLEDALVLQMELQKKLHEQLELLLLLLLLLLLLLLMLLLLLLLLLLLLLLLLLLLLLLLLLLLLLLLLLLLLLRLLSLEPRRSCDGSVACSCSCAEVFISGELY

SEQ ID NO: 6 - protein, 205 aa, unidentified
  source 1..205: note = Entransia fimbriata
AQSSSKRMPADSGAEPTGSTGSGHHSQGRMSEGVYLSSNKQRLRWTPELHELFVSAVHELGGADRSTPKGILRLMGKQGLTIYHVKSHLQKYRLAKLSGQSKYSQPATPPPQGADTGMAPLPPIRPGSSGGGAPTASVTSTVSEQDIEKDAGMGALPKSLQISEALRMQMEVQKRLHEQLEVQRQLQLRIEAQGKYLQQIIEEQQ

SEQ ID NO: 7 - protein, 228 aa, Golenkinia sp.
  source 1..228: note = Golenkinia longispicula
AGGSAKTRLRWTPELHSRFVASVNQLGGPDKATPKGILKLMGVEGLTIYHIKSHLQKYRLNIRLPEASSSGPLTSSDIGLDTPDATMSISEATMPIVSEAQVQQRVEQSSVQTLTSLTSTQAETQSTTPTASASLEPVTHFVSQPMAVVDPTSESRRFTRKDLEEALLLQMELQKKLHEQLECQRQLQHHLEAHGRYIAQLMEQEGLAHRLADLTGQPLHPGPSSSEA

SEQ ID NO: 8 - protein, 202 aa, Oedogonium cardiacum
ARAGTVKARLRWTPELHTRFVASVQSLGGPEKATPKGILKLMGVDGLTIYHIKSHLQKFRLNMRLPESTNTSQGNEAGTSSKRSKKDDLQGGDSPPFEQPKASETSTSSQPPPGALTTSTATSAPEALIHHEFPFPQFGSGSGTITRKDLEEAMLLQMEMQKKLHDQLETQRHLQLSLEAHGRYIASLIEQEGLGQQMPELS

SEQ ID NO: 9 - protein, 270 aa, Cylindrocapsa geminella
SGDYVYPAAAPGHLSQLGPGMGPGLQSMSHSMGFLAEGSHASGSHGMYPHSTIFTNTPSKDGSRKGRLRWTPELHGRFVSAVTQLGGADKATPKGILKIMGVEGLTIYHIKSHLQKYRLNIKMPEAEAGGTGQSTDTALKVESNVPAVGQARPLGAAVPPGNGSFRDMGAEVNPAAVSARADTTAGPFVGEQRGDQVTQPVRGQEQLPPVVQQSSAAPADSVNIHEVLKQHVALQRKLAEQLETQRQLQSHLEQHGRFLRELINTSASTS

SEQ ID NO: 10 - protein, 186 aa, unidentified
  source 1..186: note = Scherffelia dubia
SPSAFKPRLRWTNELHNQFIEAVETLGGHGKATPSALLKHMNMEGLTLGHVKSHLQKYRTEIRRAKEARCKVKDVLKEIKRDKASKPGAGGKALDVAREAYEDGPNARELEEAMHTQLELQRLLCDQLEAQKKLQSSLEQHTKYISVLMRNKSDVRTKPRDPPDTGSIEPGFQAVGAESGPSTSEA

SEQ ID NO: 11 - protein, 180 aa, unidentified
  source 1..180: note = Hafniomonas reticulata
KSRLRWTPELHTRFVAAVSSLGGPEKATPKGVLKLMGVEGLTIYHIKSHLQKYRLNIKMPADGKQMSGSDMSGAVLGDPNRPELPSVSSLALDTSEATHLSPHLRPHGSPAPLAATSVPSLSSLPGSIDAKGSLEQALLFQMELQKKLHEQLEAQRQLQHSLEAHGRYIASLMEAAQEGV

SEQ ID NO: 12 - protein, 200 aa, Tetraselmis sp.
  source 1..200: note = Tetraselmis chui
QNTNFQMPSGMHFPNFNPNVPDGNMPNFGSSLFPPTTFRPRLRWTNDLHNQFLESVEQLGGHGKATPSAILKHMAVDGLSLSHVKSHLQKYRTELKRAKAVRGRALNDMNQIKKGARRRAGEGSGGSSAEEGLDILGSTHEELQKQLAAKAKGPNAKELEDAMRTQLELQKMLCAQLEAQKKLQSSLEQHTKYISVLMQK

SEQ ID NO: 13 - protein, 218 aa, Volvox globator
EFLPVLGFDAYSAKPTGLGLGGLLPDPPRTSTDGASTLLQSSDFMLSMPAVPHLMQPGVGTLQPPQSAFPDLTLPGAGSLGLNSGLLHHPSGHFMGQPQRAATATAPGHGPAKSRLRWTPELHNRFVMAVNQLGGPEKATPKGILKLMGVDGLTIYHIKSHLQKYRLNIRLPGDGVQGDSAADSDMSDGEPGGDGFGGPSTVAGEMQSGLAGGGGVSG

SEQ ID NO: 14 - protein, 736 aa, Volvox aureus
  source 1..736: note = Volvox aureus-M1028
GRAALPMDKAERAASNAIGNEDDWLLEFWPEPAAADFLGPVAGAMQQQQQHPLQLDHSQLPEQVPHSGSFQMSQFGLSPPTSDYLPGLQFDAYGSKPHGLSGLGGLFHDHQRSSTNGASTLLQPSDLLFPMCGVTHALMQHPAGVAGFQQPAFPDLPLGGVGLHPGLLPGHYLSHQQRAASCPAKSRLRWTPELHNRFVASVNQLGGPEKATPKGIMKLMGVDGLTIYHIKSHLQKYRLNIRLPGETMPGDSADTDGSDGEGEAPSASMDRLDRLEATQSGMLGGEGGAGGAGGGATTAATEQTVSISAQGKSGRRSGPAGGTSCSSGSAPSATRRNLEEALLFQMELQKKLHEQLETQRQLQLSLEAHGRYIASLMEQEGLTGKLPELTEAPLGGGGASASIGSRERRASGGLGAGLSSVQQAPLGSAPPPLTTSKDRGGRGIAAGRAISGGCGALQSPATNLSGASPHLQASSGGVAGVGLQPLQPPPAAVGAAAGRQGNQQQKPQHQFQNQQQQQQQQQKQVQAVGNSILTGVRHSPLHGLPSLGGSGGGGRGSVTSVTSSSSMHFQMQQDHQRLELMRLGRLGSHPTPGSPSGNPLVDGGGGGGGAGVNEKPQHIVSNTGLAVVGESSIPLEQPVVILQDGGHSGQTAALAHQQPEPPQVHPQPALRAASGQLESGLGLGDALEGIIGESGNGGGGANGGSVVPLPDFDFGDFPDLDSGGLEHQGLLGPGDL

SEQ ID NO: 15 - protein, 162 aa, unidentified
  source 1..162: note = Ignatius tetrasporus
AKPRLRWTPELHKRFVHAVQQLGGPDKATPKAILKLMASPGLTIFHIKSHLQKFRLNIKLPDTKKEGSKATVSGQSEPFADQADNAVSMQFEEPTSEPEVAVSPSAGSSHVAYKGLLGKNLGEALVRQMELQKKLHKQLESQRHLQLSLEAHGRYIAGLIAR

SEQ ID NO: 16 - protein, 267 aa, Gonium pectorale
MDKAERQAATSIGPEDDWLLEFWPEPTVSDLPRFGPAMQPLHQPHQPLDAPQHPELLSQQQQQHQALQLGAYGLQAQAPMGSDYGLPGLGFDAFGGKAPLGMGGMLSEHPRASEGASAMLPPSDFMLPMGGVGSMPHLMQPGMGALQQHSFHDFSLGGAGLAQGMLHGHYMAHQQRAAAGPSKNRLRWTPELHTRFVTSVNQLGGPEKATPKGILKLMGVDGLTIYHIKSHLQKYRLNIRLPGEGGMQGDSPVDSEMSEGEGAAPSA

SEQ ID NO: 17 - protein, 264 aa, unidentified
  source 1..264: note = Planophila terrestris
QPSASTTGKSGRLRWTLELHKKFADAVAKLNGPDKATPKGILKLMDTKGLTIYHIKSHLQKYRSNMRTMQPAPMQAALSDAHTATHHDARSFGMQSFDAAVSASGAMHSGALTSNPLGLGLNFPQSSVSSMGAMLGGMGSAAAGPLTMTGLPPIRTDASLLRAASQGPSQGPDALSWAHAHGTSPAFARGGSPTCSVHSGAGRRDEGRGASGEGVDQSHSWTKALVKQMELQKQLHEQLSMQRQLQLNLEAHGRYMLRLVAKEG

SEQ ID NO: 18 - protein, 571 aa, Pteromonas angulosa
GGATASGGCRAGNGLGSGMSEPHEPRDGKQHLGSLDELWLDDHAVGELSWWPEPQLPLPDPLLLQGGAAGLMLGHQPQQQAQQQQPAYHGRMPPQQQQQQPSQFNNEYGYAPVPHDPYALGAHAQAPAGPSGDMSAGVSAVNYASLTPGPAIPQPHLPPQPMPQQLQQHPGYMDPSMMGGMYGGQFYPAAPQQGPAKSRLRWTPELHNRFVAAVNQLGGAEKATPKGILKFMGVDGLTIFHIKSHLQKYRLNIKMPGEGTPMPGDSDSESIDVGVRPMQQPQQQMQQPQQQMQMQQQGDTSMRSKSRGDSGDGVRGGGAVVAPASQAAGPGLSSSTSSAINRKNLEEALLLQMELQKKLHEQLETQRQLQLSLEAHGRYIASLMEQEGLTQRIPQLHGNAQMPASTANAAAAVAAAAAAAAAASSKHNDPRGAAAGSSASGQGGQSAAPSQPALEHRAASGAGAMPAHYQQQPPQQQYMQQQQQQHPVSDSGMAAAAAAAAAAAAAAGMAGPSEMYAGHSQQQHHSSQQQQQPYLQLQHRTSQQQGEHQPSSIAAAGSAWGEAASTGHGPG

SEQ ID NO: 19 - protein, 188 aa, Asteromonas gracilis
GPSKSRLRWTPELHNRFVQAVNYLGGPDRATPKGILKLVNGEGLTIYHIKSHLQKYRLNIKLPMDPSGSEYMSDSQQDVSASGEMRSSVGHVVTRRRASPMPGAAQETPQQHATQVQAVGNTQEGTSRCLDTSAVASGDKSKSLEDALLFQMELQKKLHEQLESQRQLQQSLEAHGRYIASLMEQEGL

SEQ ID NO: 20 - protein, 186 aa, Haematococcus pluvialis
  source 1..186: note = Haematococcus pluvialis-B
KSRLRWTPELHNRFVQSVTTLGGPDRATPKGILKLMGVDGLTMYHIKSHLQKYRLNIKMPAESGGQDSLSDSQDQQPPSAMEVRSSSRGPTSTPQLRAPGSSYDCSGQAPALVSAASVTAVPAPSSAGAASSGTNRRNLEDALLFQMELQKKLHEQLESQRQLQLSLEAHGRYIASLMEQEGLTQR

SEQ ID NO: 21 - protein, 111 aa, Chlamydomonas sp.
  source 1..111: note = Chlamydomonas bilatus-B
GHPGHHEQFRRQSEDKPGSSKSRLRWTPELHNRFVNAVNQLGGAEKATPKGVLKLVNVEGLTIYHIKSHLQKYRLSMKLPGDAGGAESPAESDSGLEQGQGQQPTRRRSSM

SEQ ID NO: 22 - protein, 261 aa, unidentified
  source 1..261: note = Vitreochlamys sp.
GIGHNVLALQGLPSQSPAQMLPFVPDYSGQLGAGALPPGLHLQQGVLRSSSAAQAQRARLRWTPELHNRFVTAVNALGDKATPKGILKLMGAEDLTIYHIKSHLQKYRLNIRLPSGPHADSGVSSDNDMGGMGNALSTVSSGPLGDLADFPQQHEFQTQQQRSLSMSQTQQQLSQPQAPFIPGTASSPMQAASIAPNPGGSSPTMQQMMPDQQHRMGNLEHALLVQLELQKKLHEQLEAQRQIQMKIEAHGRYLASLMASE

SEQ ID NO: 23 - protein, 259 aa, Botryococcus sp.
  source 1..259: note = Botryococcus terribilis
GMGGHQAPLGGAPIFTTSRSVSSSQGMRDPGKQRLRWTPELHARFVGAVNQLHGPERATPKGILKLMDIDGLTIYHIKSHLQKYRLNIKLPGQGGPMFEEDDMSERRSTKRRRTKARSTKRRRKARGRISDSESEEDHYESEEEENDPEEVEAEEEEDDDGGEVASRGTGAPSGVPVPVPHHPARSQPMPTASSTEDSVDADGKKATRHDRQAALTKALNDQMEMQQKLMEQLESQRRLQSQVERHTAYLRGLMEEEGL

SEQ ID NO: 24 - protein, 234 aa, Eudorina elegans
GRVALPMAKAERASGTVVCNEDDWLVEFWPEPAAADLLSAVPGAMQAQQQHPHQQLDPSQLSELLPQQTGLQMGQFSLHQTNEYLSSIQFDAFGGGKATGLAGLGGLLPDHQRSSTDGASALLQSSDFMLPMAGGLQQPAFPDLALGGVTLNPGMMPAHFLGHQQRAASGPAKSRLRWTPELHNRFVASVNQLGGPEKATPKGILKLMSVDGLTIYHIKSHLQKYRLNIRLPGE

SEQ ID NO: 25 - protein, 184 aa, Pandorina morum
GLGGLLPDHQRSSTDGASALLQSSDFMLPLGGVPHLMQPGVAGLQQSAFPDLALGGVGINQMLLQGHFLAHPQRAASGPAKSRLRWTPELHNRFVASVNQLGGPDKATPKGILKLMGVDGLTIYHIKSHLQKYRLNIRLPGETTQGDSADSDASDGEAADPSASMDRTVETQSGLGGGCGGSLA

SEQ ID NO: 26 - protein, 201 aa, Oedogonium foveolatum
ARAGTVKARLRWTPELHTRFVASVQSLGGPDKATPKGILKLMGVEGLTIYHIKSHLQKFRLNMRLPESTSTSQGNEAGTSSKRSKKDEPQGGDSPAVEQPKASETSTASQPPPAALTTSTATSAPAAALHHEFHFPQLGRGIGSITRKDLEEAMLLQMEMQKKLHEQLETQRHLQLSLEAHGRYIASLIEQEGLAQQMPEL

SEQ ID NO: 27 - protein, 235 aa, Chlamydomonas sp.
  source 1..235: note = Chlamydomonas sp.-M2762
PQRPAPKGSSKSRLRWTPELHNRFVNSVNQLGGPDKATPKGILKLMSVDGLTIYHIKSHLQKYRLNVKTPGDSAAMYDMDSDGDGEGEVTDTRPARSKGQSEATTSSGGTARGKHSNRQHQAASAPVGLPAAAPAPPVPGMTTAASLPVVSSNNRKNLEDALLVQMDLQKKLHEQLENQRQLQAQLQAHGHYIASLMQQEGMATPAETQPPAPDTKPPGLPSTSAPAGLPGPLPP

SEQ ID NO: 28 - protein, 240 aa, Chlamydomonas sp.
  source 1..240: note = Chlamydomonas sp.-M2762
GVSKSRLRWTPELHNRFAAAVRLLGGPDKATPKGILSQMSAPGLTIYHIKSHLQKYRLSSKSPGNFSLNDDSDDGLAGEGDEDTSCMASGHRQDFAAAALPGDADRRAAHPGSPRRAIVTNMEVSGSPAPSVRPQAAVMTAPRLPDAAASNRRNLEMALLRQMELQKKLHEQLEAQRHLQLSLEAHGHYIATLMQKEGYAGGPTPPEPAAGACPAPATAAAGTAVISSAVPQGLARACSS

SEQ ID NO: 29 - protein, 195 aa, Chlamydomonas sp.
  source 1..195: note = Chlamydomonas noctigama
YQMPGIVGAAPTKKGRLRWTPELHACFVNSVHQLGGFEKATPKEILRLMKTEGITLYHIKSHLQKYRHCMKLGRLGGTDSSDASENLPGDQQSPQPILDCHMPGRTDGSLEVAPSRPADGGRTTCHRHNDSTRQFSDANVQASASSCSIRRTALEEAIALQKELQKKFREQMQTQIELQARLEAHGRYIATLVER

SEQ ID NO: 30 - protein, 238 aa, unidentified
  source 1..238: note = Carteria crucifera
GTPKSRLRWTPELHNRFVNAVNQLGGPEKATPKGIMKLMSVDGLTIYHIKSHLQKYRLNIRLPAESQLTDSSTENKHELQGQSPVQEPQQQERDCGGTSAIPCELTVPTTTSGSGAVTTVPNALYTNVQASIVASQASIVASAHPPPSTAEPPVQAGPSSSERRSSPEPSSSTRKNLEEALLFQMELQKKLHEQLESQRQLQLSLEAHGRYIASLMEQEGLTHKLPELTGQTLGAPSS

SEQ ID NO: 31 - protein, 746 aa, Volvox aureus
  source 1..746: note = Volvox aureus-M2242
GRAALPMDKAERAASNAIGNEDDWLLEFWPEPAAADFLGPVAGAMQQQHPLQLDHSQLPEQVPHSGSFQMGQFGLSPPTSDYLPGLQFDAYGSKPHGLSGLGGLFHDHQRSSTNGASTLLQPSDLLFPMCGVTHALLQHPGGVAGFQQPAFPDLPLGGVGLHPGLLPGHYLSHQQRAASCPAKSRLRWTPELHNRFVASVNQLGGPEKATPKGIMKLMGVDGLTIYHIKSHLQKYRLNIRLPGETMPGDSADTDASDGEGEAPSASMDRLDRLEATQSGMLGGEGGGGGTGGGATTAATEQTVSISAQGKSGRRSGPAGGTSCSSGSAPSATRRNLEEALLFQMELQKKLHEQLETQRQLQLSLEAHGRYIASLMEQEGLTGKLPELTEAPLGGGGASASIGSRERRASGGLGAGLSSVPQPPLGSGPPLLTTCKDRGGRGIAAGRAASGSCGALQSPATNLSGASPHLQASSGGVAGVGLQPLQPPPAAVGAAAAASQSATEASASVPEQQQQQQQQQQQQQQQQQQKQQQQQNQVQAVGNCILTGVRHSPLHGLPSLGGSGGGGRGSVTSVTSSSSMHFQMQQDHQRLELMRLGRLGSHPTPGSPSGNPQVDGGGGGAGVSEKPQQVVFNPGLVVVRESSIPLEQPVVILQDGGHHGQTAALAHLQPEPRQVHPQPALRSASGQLGSGLGLGDALEGIMGESGNGGGGANGGIVGPLPDFDFGDFPDLDSGGLEHQGLLGPGDL

SEQ ID NO: 32 - protein, 186 aa, Phacotus lenticularis
PKSRLRWTPELHNRFVSAVNQLGGADKATPKGILKLMGVDGLTIFHIKSHLQKYRLNIKMPGDSSMLAGDSDSESIDPQRSLRMPEPMRSKSKGDSGDAQRGPAVPSGAPAPAGPSMPAPSPSGAGPSMPAPSSSTSSAINRKNLEEALLFQMELQKKLHEQLETQRQLQLSLEAHGRYIASLMEQ

SEQ ID NO: 33 - protein, 249 aa, Stephanosphaera pluvialis
GQALMQPQFSSQAPKPDPAAPKQRLRWTPELHNLFVQAVDQLGGPERATPKGILNLMSVEKLTIYHIKSHLQKYRLNIKCPNGDSGAAGDSDSYDQAPSGGVVEGRSLSRGSVPTLTHPHASLTSLLTGSMPSAAATSPQAVTPAASAIHVHSPPPVGCPTQPSLQLTVGTSAPGSLSQAPVSQLSNMSGGLSTTVTAANRKNLEDALMVQMELQKRLHEQLEQQRQLQLSLEAHGRYIASLMEREGMT

SEQ ID NO: 34 - protein, 585 aa, Chlamydomonas sp.
  source 1..585: note = Chlamydomonas eustigma
MADPIGQPSLPTDDPLLLTLKTGGVDEPDLDFNSWLEFWPESELPSMHSFLPQANNVPIDESYRQGFALQSAIPDIPRMQGGLLDNYDRVPTLLSAGSARDMHMLADFHGNMKSLPHHAGFSSFSGDMGPSAGSYYHEKESRHGSNSRSRLRWTPELHNRFVNSVNQLAGPEKATPKGILKLMNVEGLTIYHIKSHLQKYRLNIKMPGDMNLESCGDDSDMDERPTSTTPVDRNRRAPDLERQVSLNMDRLGRAGKVASERETLDQGRRGMDTSSGLAGPATGHTESTPPRPSTSAGTTAPLSSSSSALNRKNLEDALLFQMELQKKLHEQLETQRQLQLSLEAHGRYIASLMEQEVLASKQDGQQPSTEPSLGGGGGTTRGGITAAGLCRPPSGASDEVMIADKAGMVSTSGAASPQYLDHGGAKQGGAHLQYFSSAGSEASASAAGTGLHGLSVSASNGAHGAMLSGNQFMGMGGDSSMALMSPGKHMETHMGGTVMHGKQLHGVMRGGMVGTAAAPSPPLLPLMDAHPSHDGSPGASLLPTSLMMVQQSSPDLELLVHEAGSLASAGDCNHASKRIKLENEL

SEQ ID NO: 35 - protein, 764 aa, Chlamydomonas sp.
  source 1..764: note = Chlamydomonas incerta
MDKAERAPGGPNAASEDDWLLEFWPEPAADFPAPVAAMQPQHQDATQLQEAVPQQQGLALGAYGLAQQPSDFMQSSMPGFDAFGSGKAATLAGLPGLLPDAQRASTDGASALMNAAQQSSEYMLAAGMGGVQHLLAPSVGTALPGSGHTGFADLSMGGLAGGLAGLGGPGMMHHGQFFMQPQRAATGPAKSRLRWTPELHNRFVNSVNSLGGPDKATPKGILKLMGVDGLTIYHIKSHLQKYRLNIRLPGESGVAGDSADGSDGERSDGEGGGGGGRRATSLERADTMSGMAGAVAGGRPGGALLSPGLAGATTSTGAVGGGGGLMTEPSISRGAVLNAAGAAPAGVVAAAVGGSAGVKRPAGTSLSSGSTASATRRNLEEALLFQMELQKKLHDQLETQRQLQLSLEAHGRYIASLMEQEGLTSRLPELSGGGPAAPPAGAGGAAGGMIAPPPAQQQLQHQPQQLLQPQGSLPAGGGSGVDAGSGGGGMNLQPQHQHVHLHHHQQQLQPLARHCDTCGAGGAGGAPSGGSSMQQLQAAEQQRTELVATGRLGSMPAPASSSPLAGQQHQPLAGGAAHVHMHAHTPGAQPQPHVQRQDSYAGAAAAAAAAAAAALPQSHSHTLPADLSSNAVADPSAGPIKPEQGLSPQQQQQQQEQQEAEQLAQGLLHDSSAGAGAVSGSDGGGLGDFDFGDFGDLDGGAQGGLLGAGDLIGIAELEAAAAHEQQLQQQQQQQQQQQQQQQQLQGQEQEQLDADRAKRQRLEQ

SEQ ID NO: 36 - protein, 741 aa, Chlamydomonas sp.
  source 1..741: note = Chlamydomonas schloesseri
MDKAERAAGCTNAASEDDWLLEFWPEPATDFPAPGAAMQPAHQDATQLPETIPQQQGLALGAYGLTQQPADFMQSGIPGFDAFSTGKAPGLAGLPSLLPDPQRASTDGASALMTAAQQPSEYMLPPAMGSVPHLLAPSVGTVLPGTGHTGFPDLSMGGMPGGLAGLGGPGMMHGQFFMQPQRAATGPAKSRLRWTPELHNRFVTAVNQLGGPDKATPKGILKLMGVDGLTIYHIKSHLQKYRLNIRLPGESGLAGDSADGSDGERSDGEGGGGGGAMGRRASSLERADTASGMAGPGAVAPGRAGSTPGGQLLSPGVGATAGAMAAAAEPSMSRGAVLGASGAAAAAAPAAAGAAAGGVKRPAGGPSLSSGSTASAARRNLEEALLFQMELQKKLHEQLETQRQLQLSLEAHGRYIASLMEQEGLTSRLPELSGGAPAAPPAAPGGMLAPPPQPQQQQQQQQLLQPQGSLPAGGTTAGLDAAAATGCGGGEGPQVQQQQQQQHHVRHCETCGAGGAPSGGSSMQQLQAAEQQRNELAAAGRLGSMPAAASSSPHAGHAPLQPPGAHLHVPTPGMHQQQQQHIQPQDSYAGAAAAAAAQASPAALPQSHSHTLPGDMSSSGVMEQPGPGLVKAEPGLSPQQQQQQPQMQQDADQGLLGDGGAGAAGVSGSDGGGLGDFDFGDFGDLDGSAQGGLLGPGDLIGIAELEAAAAHDQQQQLQQLHQQEEQEQLDADRVKRQRLEQ

SEQ ID NO: 37 - protein, 541 aa, unidentified
  source 1..541: note = Chromochloris zofingiensis
MDQRREPAAASRDADTADINWLEFWPESEFKIDGAPAGGSMDPSLGTLGGLGDYLGSNLQHPQLATAAPLQLTLPGEFGSAQGLPLLSSLEAYQGSGDLNVLQSTQPGQLPQLLSSAPLAGHSLSSSYGSDPSGFTANLTSPALYPTGSYLAQNSKPGLPPKTRLRWTPELHSRFVSAVHQLGGPDKATPKGILKLIGVDGLTIFHIKSHLQKYRLNIRLPESGRSDSQGGSEPLEGGSGADSRMRAPSSTQTQAKLGLSNQLESGQAGTEATPSATAASSQDYAAAHTVSRPRSSNAGPSSGAGKAAVGDQLPAAGGSGSYSTVGQGGAKPAAQKQQQLKQQLTAGLPISAVTRKDLEDALLLQMELQKKLHEQLESQRQLQLSLEAHGRYIASLMEQEGLGHRVPDIAQLTGVQPEHLQHKEQQAGGVPLSFTQSLTEDLNVDDSALHMFPGGHSAGPSHRHQHATEQGLPDSPHLLLNFPELNELADVPQAGAGSNMLLPHMPMGQQNHMQPTHKRQRLDEGGSAQNHGSHSGSRQQH

SEQ ID NO: 38 - protein, 492 aa, Coccomyxa sp.
  source 1..492: note = Coccomyxa subellipsoidea
MQQNQHYNWVSQHVSVEDHQEPHTQHQLPQTVGHEHLTAAHAAFSDFGQHRGDQAIAGITGDQAELLEAISGTETELGLPYTALDHLQPHHPQNDLHQHGVGYGMERNPSEPTQSQGKDAKPRLRWTPELHARFVSAVASLEGPDKATPKSILKLMAVEGLTIYHIKSHLQKYRLNVRLPGESGDMISGPDESEEPSRRKRRSRSHGQASSRRRSSRQRKRRRSSDEDSDEDDMEDEDMDDDENFEEGISRARPGTSVSGINGSSPHGGSPRGGSPRGVSPRGRSPRGGSPHGGGNSMACLQVEPLEVPDLDPEKQHSLEEALLKQMDMQKRLHEQLEEQRRLQLSLEAHGRYITSLIQKKGLEGLPPQTKEALDAALVPPQGSGLSTLTHNTAPQWTPSVSEGSSLAQQVGHVMHHSTAFMLGSASATDPESSLLLDTNMQAAAAVWDPSQAHGLDQSGSKQLYEEPKELYEEPGQLYEERGGHVKPEEQL

SEQ ID NO: 39 - protein, 516 aa, unidentified
  source 1..516: note = Symbiochloris reticulata
MDTHDIAPALPERSLEWLEFWPEAEFRAEDHSMNSNPFCWVQHDDQQPSSGAKASAGPVSAAQTSEPGMLFPGQLQPVSAALLSHFTEVHTGVQADVSHSFLPPDYMQPHFCGSDLPEAAHSMPLASAAQQAPVFGATANEPSSAGAGSSQAGKPRLRWTPELHSRFVAAVNHLGGPDRATPKGVLKLMLVEGLTIYHIKSHLQKYRLNIRLPGDSGPVGSLSGSRKKRKRSRRARSSDLEDEEEEDDMDEADSMEDMLPGDELHGRQQAVGEAGLALDAALPEQGNAQVPGQQPEQQPNAQRQRDLEEALILQMDMQKRLHEQLESQRQLQLSLEAHGRYISSLIEREGLQSKLPAGTHAAMQSGLPRLPEASLGMAAGMCGPADGSGAGTIAPGTSGGMSWGQMTHVTLPHSAESPPLLSHTSRTGATAADAGQFLMVGDPGDLGPLPSMLLDTDLQAAAAVWDDGMHRPRKHAPNGHLEHASGLDEGLFDQHEGEEHGRLQRRRQPSSRLRQS

SEQ ID NO: 40 - protein, 802 aa, unidentified
  source 1..802: note = Edaphochlamys debaryana
MAKAEGRPGTIVGSEEDWLLEFWPEPTLELSGPAAMQPQQSPSLDAPITDLQQLAPQQTSQQAQQPGGLPLGQYALASAADYLQTAQHALSAYDPYRTKSAPPLPLGLLPDRPSDCASGLLPPPAGGEYLGALGTAQGGLGPVPHPLMAPGAVSGLQQGQPGGGYGDLGLGAMGMGMGGLGLQQGMLHPHAHYFAAPPRAAAGPSKSRLRWTPELHNRFVQAVNTLGGPDKATPKGILKLMGVDGLTIYHIKSHLQKYRLNIRLPGDSAAGPQGDSADDSDAEGGGGGTTATGMAAAPSMSLDRGGMETTSGLLGRRLGSNAATAAAAAGFLAGGGGGGGGGMAEPSLSNSIAAAQVAQQQAAAAAAAQMAAARPAGGSTSSGSTPSATRRNLEEALLFQMELQKKLHEQLETQRQLQLSLEAHGRYIASLMEQEGLTSRLPQLSSGDGPTAQLALPGPGGEGGGDGLQRQPSGIGGGGGGPQQGGPLVGATGQGVDHAGLGGVGPDGRRISSQGLGAPSPQALLPFQLSSAGQPTGRHQLGMQPSPQHLPGPGGDGGGGGGPGDEHQRRRSEIAYDGTGGSGLTGGASGGSSVQQLAVAEAQRHDLMRAGRLGSMPSAAAAALQAAGSNSLPQQHMYSPAQQDSLGLSQQQQQQQAQADAQAHAQAHAAAQEHAAAAAVAAGMQLSMAHAPSGSGLGDGGGGLGDGGGGLGDFDLADFVGDLDASGVAALEGQGFAGLQGGLQGDSEMGLLAGIGDDLAAAAAEAQAQGLVSPRRGSSGGEDSGRSKRARLQGSSSGEGQG

SEQ ID NO: 41 - protein, 607 aa, Enallax sp.
  source 1..607: note = Enallax costatus
MDPGPNHSLGPLEPDHCDLGYLEFWQESEFKLEPAAHHTLGMDHLGDHFLAAGAVGGSAGQYGQLGLAAGPDPYSNQGIPLVPALNDQHFQAGDVSLLSTSTGQGNQVPQLLTTPALESYTSSYGADPLSSMPSGAMLYSSGAFAMPGSKGSSAFDAPSNKTRLRWTPELHSRFVSAVNQLGGPDKATPKGILKLMGVDGLTIFHIKSHLQKYRLNIRLPEGAQPAMSTGSMQEGDAAAAAVDSAADTQTAVMSGAQAAAAQQPSQQQQQRGQQDKSGQQDKPTQQQQQQAPALVPQPSSSAGRAAASLSPLIREGSTTSIPGLSSGAVPDMQAPLLPPGTGSGGPAGQQQQQQSQQQPPPPQQQLKQAQQQPLQQPQQHARPVPETAAAAGGAAATDENNDAAIKHSTRRDLERALLRQMELQKQLHEQLEMQRALQHSFEVHQRYIHSLMEQEGLAHKIPEMSAALGAVAAATATAPPGSVVSEAMPVQPAQPSNNSQPLQQQQQQQPPGAQAGAAVPAPGQQQQQQVLQLPQKQQHTGHASANDAAPVAAAAVTDQFLSDAELLMGFPDLQHDTGDIDPIQQHLLGDEAAGGPPKRQRMSGQDV

SEQ ID NO: 42 - protein, 596 aa, unidentified
  source 1..596: note = Mesostigma viride
MNRPPVGSASRTADQQLSSSAEVQPRTVQKLPTTVDELINQEWPIWGELAPNDDSITTCWTDLLTGPPPKNQDMHRPQHATIQDDTSPGLYLARQQYLPGMGTLPPGGVPPLCAPPGLMDGGGMNLVPGMQASMAAAQSQQPPKQRLRWTPELHDRFVNAVQNLGGADRATPKGVLRVMGVQGLTIYHVKSHLQKYRLAKFLPEEGGNSSKSLGGSKRDTDSDNDDASDGDPLKMADLKAGATELLTGEDGSVNIEEALRMQMEVQKRLHEQLELQRALQLKIEAQGRYLQQIMEEQRNAALARRAQAGGAASGATTQGQATSAGQAASASSSRSSAGGGGKGPEGAAAPAAGEGGAGADSISHAADGARAGAEAGDAQRQAVASPSGAPVFAASGVHDADGAGATCPAVGAAGGHAPSPALVPKTESVACGGSLAMPDALASLPGGGGHHLGASGKLPGCELPLPSWSEPGAALLTANGGILPFPSKVEGRNLPQLSLPSHLLLGVEDVDDGGGGGGGGGGGGQSLQSGVGVGSKRAYDEMMGGGIAMEDGAGDRLHTDGSGLPTGGSLLPDDASLLAAQGGHASGGADPAPHML

SEQ ID NO: 43 - protein, 563 aa, unidentified
  source 1..563: note = Raphidocelis subcapitata
MAERTPGSPAGEGDEAVLAGLAGWLNDELSYWPEWPVGPPAPPLDPQAHCDGPVIALPGAHCPMEQRQVAAGPPGPHGGAGPHAVAQPQQQHPALQAGQGHALDAFQSYQATAYGMQLAVHAQQGGFDPGMLGAAGALAPGALFGVPPAYGMAGGKPGAMAGGNKSRLRWTPELHASFVAAAESLGGADKATPKGILKLMAVPGLTIFHIKSHLQKYRLNVRAPDGTEGASDGGGESAVEGASGEGGATVRMGALRAESLDATAPSSALALPPTALGASPAVGVKPEHPEVDAHSLLKQQQHAVPASTTSTCAGLSSATGLEAAAAAGGAGSEAAAGGPSTARRRNLEDALQLQMDLQRRLHDQLEAQRALQLSLEAHGRYIARLMEQEGLGHRLQDLAAITAPGPGAGAEAEAAPGGGDGGGAAGSGGAGSGGAGPGGAPAAAPASEANSSGLRAAAGGCGGGRSVAGGCCDGALPLARAGSSALDSSDHPAEPHQQPARWQQPTPPPSASGRRDDRSQDQRLHAAAGQLLAWGRSAPPPHDAAGLDAAGAPQGKRPRLSGA

SEQ ID NO: 44 - protein, 442 aa, unidentified
  source 1..442: note = Symbiochloris reticulata Africa
MLFPGQLQPVSAALLSHFTEADVSHSFLPPDYMQPHFCGSDLPEAAHSMPLASAAQQAPVFGATANEPSSAGAGSSQAGKPRLRWTPELHSRFVAAVNHLGGPDRATPKGVLKLMLVEGLTIYHIKSHLQKYRLNIRLPGDSGPVGSLSGSRKKRKRSRRARSSDLEDEEEEDDMDEADSMEDMLPGDELHGRQQAVGEAGLALDAALPEQGNAQVPGQQPEQQPNAQRQRDLEEALILQMDMQKRLHEQLESQRQLQLSLEAHGRYISSLIEREGLQSKLPAGTHAAMQSGLPRLPEASLGMAAGMCGPADGSGAGTIAPGTSGGMSWGQMTHVTLPHSAESPPLLSHTSRTGATAADAGQFLMVGDPGDLGPLPSMLLDTDLQAAAAVWDDGMHRPRKHAPNGHLEHASGLDEGLFDQHEGEEHGRLQRRRQPSSRLRQS

SEQ ID NO: 45 - protein, 634 aa, Tetradesmus sp.
  source 1..634: note = Tetradesmus deserticola
MDSGAHDLGDHTGDWLEFWHESEFKLDGVSTAAAQPGQHAPMDLPGGLGDFFLPSGSMLPQPHSGDAQQLVLAPAGDPYAGSLTMLPGLEQQQQHYKGPDLSFMSTSSGAAGQMTQLMPPTAQLESYTSSFSSDPTLSGMHSAPMLYHAASFQLPGTRSGSLQEAPAGKTRLRWTPELHSRFVQSVNSLGGPDKATPKGILKLMSVDGLTIFHIKSHLQKYRLNIRLPETSEMGAQPANSSGSPDQEATAATDSAADTHATLATSTINPSAAAAVAAGAAAPTAAVAPASASAGGGSSLQQQQQQQSLVPTSQQQQQQQPPPPQQQQQQRILSGVEQLSGASPLQLTTSGVLEMPDSAASAAQQQQQQQQQQQQQPTGAAADAAEDSLHMKSDTRRDLERALLQQMHLQKKLHEQLETQRQLQHSLEVHQRYIHKLMEQEGLAHKIPEMSAAFNAGALPPPGSVVSEAMPGQPLAVGTAPQQQQQQQQQASSAAPPLQRHHSLPHQQQLHTGVGNSDAAAGVGTSKRSSSHHHHHHHQHHQQHHPQQQHHPQPMQQQQEPGQDAAGIDPLPGSCGNLLSDHELLLGFPELRDSGDEGGGMGLLSEPGQPQGKRQRLLTPDIAKWPSVDSAEGQH

SEQ ID NO: 46 - protein, 475 aa, Tetraselmis sp.
  source 1..475: note = Tetraselmis striata
MNIRHDDDAAAASVQVRERVPWSQGIRGVEGAVVVVFILARFTPTAPQTRQTLQPPYRQLARHREDPATSPDPRTGRPAMDLNEDADAELNFFKAMEAFSPPSFEGGEAEDHLHGLSVPGLPHMAGATDTHAHNPPASTGDGSQSATVHNTSGARGHDLLVNNGHSMWEPLSFEEVMRNGGVNPSQASSLASTSTAATELLMHRGNTFLPSGNGGGRQAPPGQFGMGGMPSMMAFGAPQQQQQHQQHQPTPQQQPPQRNGSEDGMQHFGGLFPQSAAFRPRLRWTNDLHNQFLDSVERLGGTDKATPSAILKHMGVDGLSLGHVKSHLQKYRTELKRAKAVRGKAMDDMHQMKKGARSKAAAADVAAEAAEVVAEASGSAEAGLEQLGATQRELQRQLAARAASGPNAKELEEAMRTQLELQKMLCAQLEAQKELQRSLEQHTKYISVLMKRQSGDDLHAHGEGDTAGEHEMSKA

SEQ ID NO: 47 - protein, 536 aa, Trebouxia sp.
MDNDTIDWLDLDYWPEKDSKKPADMDNSFAWLAQQAQPLSGQPLPGSQYQVQPHVMQPHDGLLYHDTFHPHSTAGSLLSDLSGDLLDTTAVDISNAQFEAIPSQSPHQQSNMQLRSDTAHNGAPQPLQDMIQAPVFGRSTSSMSQQAGNNSQSAAAQAAGKPRLRWTPELHTRFVGCVSQLGGPEKATPKGIMKLMSVEGLTIYHIKSHLQKYRLNIRLPESEQVEMSEAVSGEHEGRKSQRGKRRSTRKQRKRSKRSSSRRRALEKSDGDDDEADDLDDDQFDEEEGDNELDGHAASSGVGEASSMLDGVTNREEDAQREVQRQRNLEQALLIQMEMQKKLHEQLESQRQLQLSLEAHGRYITSLIEREGLQHRLLPQLVAAAAPSLARTVPALAALAASMPPGSSGQISDQQTHYMPLSASGASEFSPQQLLAGRFSSLPNSVNLNQDPSPGATDAARSLDVSPSSLSRHVSGAVPRNPFGTINQAAFGEPSSPGLLLNTDLQAAAAAWDDQQRHILTGPGSRPLDGMPAVPGQ

SEQ ID NO: 48 - DNA, 1080 bp, Monoraphidium sp.
  source 1..1080: mol_type = other DNA; note = Monoraphidium neglectum
atgcagcaggacctgctgggtggccctcctgggcccatgcatcatgatgaacagcacgaaatgttgcagcagcagccgcagcagcagcaggcacaaccccaggcgcagcagcagcaacagcagcagcacccgcagcagcagcaccagcagcaccagcacctgcccggcaaggccgcctttcccccgggcatgggtgtgcctggcatggatcactttcacggcacaccctacggcatgcaagctgtgcccatgcagccaggccactttgaacatctgctcaacgcaatgcccgtgacgggtcactcgttgagctcgtcgttcgcaacggacaacgtgcacatgagcggagcccagcccacgctctatctggccgcgggtttcaacatggccggttccaagccaggcgtgagtgcggctggctcggctggcggcggtggcggtggcggcagcaagacgcgcctgcgatggacgcccgagctccactcgtcatttgtgcgctccgtgcagcagctgggagggcccgacaaggcgacgcccaagggcatcctgaaagccatgaacatggatggcctcaccattttccacatcaagagccacctgcagaagtaccgcctgaatgcgcgcgtgcccggcgccagcagcgttgacggtggcagcgatggctgcgcggctggggactcggcagagggcaacagcggcagccgaccggcctctgcagctttagacgggctgggcagcgtccccgtgtctgccctgacgcgcaagaatctggaggacgccttggtgctgcagatggagctccagaaaaagctccacgagcagctcgagctcctgctgctgctgctgctgctgctgctgctgctgctgcttatgctgctgctgctgctgctgctgctgctgctgctgctgctgctgctgctgctgctgctgctgctgctgctgctgctgctgctgctgctgctgctgctgctgctgctgctgctgcggctgctgagcctggagccgcgtcgttcctgcgacggcagcgtcgcgtgcagctgcagctgcgctgaggtgttcatctcgggtgagctctactag

SEQ ID NO: 49 - DNA, 615 bp, unidentified
  source 1..615: mol_type = other DNA; note = Entransia fimbriata
gcccagagcagcagcaagaggatgccggctgactcgggggcagagcctactgggagcaccggcagcggccaccattcgcaggggcggatgtccgaaggggtgtacctttccagcaacaaacagcgcctccgctggacgccggagctgcacgagcttttcgtctctgctgtgcacgagctaggaggcgcagaccgctcgaccccgaagggcatcctccgcctcatgggcaagcaaggccttaccatctaccatgtgaagagccacctccagaagtaccgtcttgccaagctcagcggacagtccaagtacagccagccagccaccccgccccctcaaggcgccgatactgggatggcccccctgccacccatacgccccgggtcctccggtggtggcgcgccgacagcctccgtcacctccactgtctcggagcaggacattgagaaggatgcgggcatgggagctctgcctaaatcactgcagatctcagaggcactccggatgcagatggaggtccagaaacgcctccatgagcagctcgaggtccagcgccagctccagcttcgtattgaggctcaggggaagtacctacagcagattatagaggagcagcag

SEQ ID NO: 50 - DNA, 684 bp, Golenkinia sp.
  source 1..684: mol_type = other DNA; note = Golenkinia longispicula
gcaggtggatcggctaagactcgactgcgttggacaccggaattgcacagcagatttgtggcctcggtcaaccagcttgggggaccagacaaggccactcctaaagggatcctgaaactgatgggagttgaggggctcacaatctaccatattaagagccacttgcaaaagtaccgccttaatatccgtctccccgaggcatcgtccagtggccccctgacctcctccgatatcggtctagacaccccggacgcgacgatgtcaatatcagaagcaaccatgcccatagtgtctgaagctcaggtgcagcagcgggtggagcagagctctgtacagaccctaacatcattgacgagcacccaggctgagacgcaatctacaactcccacagcttctgcttcactagagcccgtcacccactttgtctcccagcccatggcagtggttgaccccacctcagaatccaggaggttcacccgcaaggacctggaggaggcattgctgctgcaaatggagttgcaaaagaagttgcatgaacagctagagtgtcagcgccagttgcaacatcatttggaggcacatgggcgctatatcgctcagctcatggagcaagagggcctggcccatcgcctggcggatttaacaggccaacccctgcatccgggacccagtagctcagaggca

SEQ ID NO: 51 - DNA, 606 bp, Oedogonium cardiacum
  source 1..606: mol_type = other DNA
gcccgtgcaggcactgtgaaggcaaggctgcgctggactcctgagcttcacacacgcttcgtggcatctgttcaaagtttaggcgggccagaaaaagcaactcccaagggcattttgaagctgatgggagtagacgggctaaccatttatcatataaagagccatttgcaaaagtttcgtcttaacatgcgtctgcctgaatcaactaatactagtcaaggaaacgaggctggcacaagcagcaaacgaagtaagaaagatgatctccaaggaggtgattcccctccttttgagcaaccgaaagccagtgaaacctctacatcttcacaaccaccaccaggtgcattgaccacctccacagcaaccagcgcacctgaagctctaatccatcatgaattcccgttccctcaattcgggtctgggtctggtactatcacccgtaaggacttggaggaagccatgttgctgcaaatggagatgcagaagaaactgcatgatcagttggagactcagcgacacttgcagctcagtttagaggcacatggcaggtacattgcaagtcttattgagcaggagggtctagggcaacagatgccggaattgagt

SEQ ID NO: 52 - DNA, 810 bp, Cylindrocapsa geminella
  source 1..810: mol_type = other DNA
agtggtgattacgtatacccagcagccgcacctggccacctgagccagctagggcccggaatgggccccgggcttcagtccatgtcacatagcatgggcttcttggctgaaggctctcatgcgtctggctctcacggaatgtacccgcactcaactatttttacgaacactccgtcgaaagatggatcgaggaaaggccgactaagatggactcctgaactacacggtcgctttgttagtgctgtaacccagcttggtggtgcggataaagcaactccaaaggggattttgaaaatcatgggtgtagagggcctaactatttaccatatcaagagccatctgcagaagtatcgtttgaacatcaagatgccagaagcagaggcaggagggacaggccagtcaaccgatactgcgttgaaggttgaaagcaacgtgccagcagtaggtcaggccaggccgctaggcgctgcagttcctccgggcaatggttccttcagggacatgggtgcggaagtgaacccggctgctgtttctgcacgtgctgacaccacggctggtccgtttgttggagaacagcgtggagatcaagtcacacagcctgtccgcggtcaggagcagctgccgccagtggtccagcagagcagcgcggcaccagcggacagtgtgaacattcacgaggtcctcaagcagcacgtagcactgcagcgaaaactggcggagcagcttgagacgcagcgccagcttcagtctcacctggagcaacacggcaggttcctgcgcgagttgataaacactagtgccagcacgtcg

SEQ ID NO: 53 - DNA, 558 bp, unidentified
  source 1..558: mol_type = other DNA; note = Scherffelia dubia
tccccgtctgcattcaagccgcggctacgctggacaaacgaactgcacaaccaatttatagaagcagtggagactcttggggggcatggcaaggccacaccatcagcactgctgaagcacatgaacatggagggcctgacccttggacatgtcaagagccatctccagaagtaccgcacagagattcgccgtgccaaggaagctcggtgcaaggtcaaagatgttctcaaggagatcaagcgggacaaagccagcaagcctggggcagggggcaaggcgttggatgttgcaagagaggcgtacgaggatgggcccaatgcgagggagctcgaggaggctatgcacacccagctggagctgcagcgactgctgtgcgaccagcttgaggcccagaagaagctgcagagtagcttggagcagcacacaaagtacatctcggtcctcatgcggaacaagtcggatgtgcgcaccaagcccagggacccaccggacacaggctccattgaaccgggttttcaggccgtgggggcagagagcgggcccagcacttcggaggcc

SEQ ID NO: 54 - DNA, 540 bp, unidentified
  source 1..540: mol_type = other DNA; note = Hafniomonas reticulata
aagagccgtctccgctggacgcctgagctgcacacccgctttgttgccgctgtatccagcctgggtggcccagagaaggccacgcccaagggtgtgttgaagctcatgggcgtagaaggcctgaccatctaccatatcaagagccaccttcaaaaataccgcctcaacatcaagatgcctgcagacggcaagcaaatgtccggcagtgacatgagcggggccgtactaggagacccgaaccgaccagagttaccctcagtcagcagccttgccttagacacaagcgaagccacacacctttcaccgcacttgcgaccacatgggagcccagctcccttggcggccaccagcgtcccgtccctcagcagcctgccgggctcgatcgatgcgaagggcagcctagagcaggcgctgctgttccagatggagctgcagaagaagctgcatgagcagctggaggctcagcggcagctgcaacacagcctggaggcgcatggccgctacatcgcctcactgatggaggctgcgcaggagggcgtc

SEQ ID NO: 55 - DNA, 600 bp, Tetraselmis sp.
  source 1..600: mol_type = other DNA; note = Tetraselmis chui
cagaacaccaacttccaaatgccctcgggcatgcactttccgaatttcaacccaaacgtgcccgatggaaatatgcccaacttcggttcaagtttatttccaccgactacgtttagaccgcggctgcggtggaccaacgacctgcacaaccagtttcttgagtcggtggagcagctgggtgggcacggcaaagccacgccatccgcgatccttaagcacatggctgtcgacggactgtcgttaagccacgtgaaaagtcacctccaaaaatataggaccgagctgaagagggcaaaggcggtgcgggggagggcactgaacgatatgaaccagatcaagaagggcgctcgccgtagggcgggcgagggaagcggagggagtagtgcggaggagggtctggatattttggggtctacgcacgaggagctgcagaagcagctggcagcgaaggcgaaaggccccaacgcgaaagagctggaggacgccatgcgcacgcagctggagctacagaagatgctctgcgcgcaactcgaggcgcagaagaagttgcagagcagtttggagcagcacacaaagtacatatcggtgttgatgcaaaag

SEQ ID NO: 56 - DNA, 654 bp, Volvox globator
  source 1..654: mol_type = other DNA
gaattcctgcccgtccttggctttgacgcatacagcgcaaagccaactggactgggcttgggcgggcttctaccagacccgccccgaacatctaccgatggagcatctacgctgctccaatcctcagattttatgctatccatgccagccgtaccgcacctgatgcagcccggcgtaggaactttgcagcccccgcagtccgctttcccggacctcacgctcccaggcgccggtagcctggggctcaactctgggctactccaccacccgagtggccacttcatgggtcagcctcagcgcgcagccactgccacggcccccggtcacggacccgccaagagccgtcttcgttggacccccgagctccacaatcgcttcgtcatggccgtcaaccaactgggcggaccggagaaagcaacaccaaaaggcatcctcaagttaatgggcgtggatggcctcacaatataccacattaagagccacttgcaaaaataccgtttaaatatacgtctaccaggcgacggggttcagggagacagtgcggcggattcggacatgtcggacggggaaccgggcggcgacggattcggcgggccatccacggttgcgggggaaatgcagtctgggctagctggcggaggcggcgtcagtggg

SEQ ID NO: 57 - DNA, 2208 bp, Volvox aureus
  source 1..2208: mol_type = other DNA; strain = M1028
gggcgtgctgccctccccatggacaaggctgagcgcgcagccagcaacgcgattggcaacgaggacgactggctcttggagttctggccggagccagctgcggcggacttccttggcccagttgcgggcgcgatgcagcagcagcagcagcacccgttacaattggaccattcacagctgccagagcaggttccccactctggaagttttcagatgagccaattcggcctcagtcctcctaccagcgattacctcccggggctccagttcgacgcatacggcagcaaaccgcacggtctcagcgggctcggcgggctttttcacgaccaccaacgctcctccaccaacggtgcatcaacactgctccaaccctccgacttattgtttcccatgtgtggggttactcacgcacttatgcaacaccctgcgggcgttgcgggcttccaacagcccgcttttccagacctgccgctcggcggggtgggtctgcacccggggttgcttcccggccactatctgtcgcatcagcaaagagcagcctcgtgccccgcgaagagccgcctccgctggacccccgagcttcataatcgcttcgtggcctcagtgaaccagctaggcggccccgagaaggccactccgaagggtatcatgaagctgatgggcgtagacggcctcaccatataccacatcaagagccacctgcagaagtatcgcttgaacatacggctgcctggggagacgatgcccggcgacagtgcagacacggatggctccgacggcgaaggcgaagcaccttcagcgtcaatggacagattggacaggttggaagcaacgcagtcggggatgttggggggagaaggtggcgccggtggcgctggaggaggggccacgactgccgcaacggagcaaacggtgtccatcagcgctcagggaaagtctggtcggcgctcgggtcctgccggtggtacttcttgcagcagcggcagtgccccctcagctacgcggcgcaacctcgaagaggcgcttctgttccagatggagcttcagaagaagctccacgaacagttggagacgcaacgtcagctgcagctcagtttggaggcgcacggccgctatatcgccagcttgatggaacaggagggccttacggggaagctgccggagctgactgaagccccgctgggtggtggcggcgccagtgcttccattggaagccgggagcgccgggcttccggcggcctaggagcggggctgtcatcagtacaacaggcgccgttgggaagcgcgccgccaccactcaccacgtctaaagaccgtgggggaagagggatagctgccggccgcgctatcagtgggggctgtggcgcgcttcagtctccggccacgaatctgagtggggcttcaccccatctccaggcttcgtctgggggcgttgccggcgtcgggctgcagccgctacagccgccacctgctgctgtgggggctgcagccgggcggcaaggcaatcagcaacagaagcctcagcatcagttccagaaccagcaacagcagcaacaacagcaacagaagcaagtgcaagcggttggcaatagcattctcactggtgtacgacatagccctctgcacggattgccatcacttggcggtagtggtggcggcggccgcggaagtgtgaccagtgtgaccagtagtagctcgatgcattttcagatgcagcaggaccatcagcgtttggaacttatgcggttgggccgacttgggtcgcacccgaccccggggtcaccaagcggtaatccgctggtcgatggtggcggcggcggaggaggagcaggagtgaatgagaagcctcagcacattgtttccaacactggcctggcggttgtgggtgagagcagtatcccgctggagcaacctgtagtgatactgcaggatggtggccattctgggcaaacagcagccctagcgcatcagcagcctgagccgccgcaggtgcacccgcagccggcattgagggcagcgtcgggacagctggaatctgggctcgggttgggggatgcattggagggaattataggcgaaagtggaaatggcggtggtggtgccaacggtggcagtgtagtgccgctgccggacttcgactttggagattttccggatttggatagcggaggattggagcatcagggtttgttgggacctggtgacctg

SEQ ID NO: 58 - DNA, 486 bp, unidentified
  source 1..486: mol_type = other DNA; note = Ignatius tetrasporus
gcgaagccgcgattgcgatggacgcccgagctgcacaaacgctttgttcatgcagtgcagcagctcggcggtccagacaaggcgacgcccaaagccattctaaagctcatggcatcacctggcttgacaatttttcacatcaaaagccatttgcagaagtttaggctcaacatcaaactccctgatacgaagaaagaaggttcaaaagcgacagtatccggccaatcagagccgtttgcggaccaggctgacaatgctgtcagcatgcagtttgaggagcccaccagcgaaccggaagttgcagtctcgccatcagcaggcagctcgcatgtcgcttacaagggtctgctcggcaagaaccttggcgaagctttagtgcggcagatggagctgcaaaagaagcttcacaagcagctggagtcgcagcgacatttacaattgagtttggaagcgcatggtcgctacatcgccggtctgattgcgcgc

SEQ ID NO: 59 - DNA, 801 bp, Gonium pectorale
  source 1..801: mol_type = other DNA
atggacaaggctgaacggcaagccgccacctcaatagggcctgaagacgactggctgctggagttctggccagagcctacggtttccgacctgccgcggtttgggcccgcgatgcagcccctgcaccagccccaccagcccctggacgcaccccagcatccggaactcctctcacaacagcagcaacaacatcaagccttgcaactaggagcgtatggcctgcaagcacaagcgcccatgggatcggactatggcttgcccggactcggtttcgacgcgtttggcggcaaggccccgctgggcatgggcgggatgctgtccgagcacccgcgcgcttcggagggagcttccgccatgctgcccccatcggactttatgctgccaatgggcggcgtgggcagcatgccgcacctcatgcagccggggatgggtgcgctgcagcagcactccttccacgacttcagccttgggggcgcagggctggcgcagggcatgctgcacggacactacatggcccaccagcagcgagcagcggcgggcccctcgaagaaccgtctccgctggacgccggagctgcacacccgcttcgtgacctcggtcaaccagctaggaggccccgagaaggccacccccaagggcatcctcaagctgatgggcgtggacggcctcaccatctaccacatcaagagccacctgcaaaagtaccgcctcaacatccggctgccgggcgagggcggcatgcagggcgactcgccggtcgactcggagatgtcggaaggggagggcgccgcgccgtcggcg

SEQ ID NO: 60 - DNA, 792 bp, unidentified
  source 1..792: mol_type = other DNA; note = Planophila terrestris
cagccgtccgcgagcaccacagggaagtcagggcgactccgatggacgctggagctgcacaagaagtttgcagacgcggtggcgaagctgaacggccccgataaagccacgccaaagggcatactgaagctcatggacaccaaaggcctcaccatttaccacatcaaatcgcacctgcaaaagtacaggtccaacatgcgcacaatgcagccggcgcccatgcaagcggcgctctccgacgcgcacacagcaacacatcacgacgcgcgcagctttggcatgcagtcctttgacgctgcggtctcagcgtccggggccatgcattcgggcgcgctcacatcaaacccgctgggtttgggtttgaatttcccgcagagctcggtcagcagcatgggcgcgatgctcggcggcatgggctcagcggctgcgggcccgctcacgatgactgggctgccgccgatccgcaccgacgcgtcgctgctgcgggccgcgagtcaggggcctagtcaggggcccgacgcgctgagctgggcgcatgcgcatggcacctctcccgcgtttgcgcgcggcggctcaccgacgtgcagcgtgcacagcggggcggggcggcgggacgaggggcgcggggcgtccggcgagggggtggaccagagccacagctggaccaaggcgcttgtcaaacagatggagctccagaagcagcttcatgagcagctctcgatgcagcggcagctgcagctcaacctcgaggcgcacggccgctacatgttgcggctcgtggccaaggagggc

SEQ ID NO: 61 - DNA, 1713 bp, Pteromonas angulosa
  source 1..1713: mol_type = other DNA
gggggcgcgacagcctctggaggctgccgggcgggtaatgggctggggagcggaatgagcgagccacacgagcctcgggacggcaagcagcacttgggctcgctcgacgagctttggctggacgaccacgccgtaggcgagctttcctggtggccggagccccagcttccactaccagacccactgctgctgcaaggtggcgcagcgggcttgatgctgggccaccagcctcagcagcaggcccagcagcagcaacccgcctaccatggccgcatgcccccgcagcagcagcagcagcagccctcccagttcaacaacgagtatggttacgcgccagtgccccatgacccatatgcactgggggcgcacgctcaggcgccagcagggccctcgggagacatgtcagcaggggtcagtgcagtgaattacgccagcttgacccctgggcctgcgatcccccagccacacttgccaccgcagccaatgccacagcagctgcagcagcaccctggctacatggacccgtcgatgatgggcggcatgtatggcggccagttctacccagcagccccccagcaagggcccgccaagtcgcgactcaggtggacccctgagctgcacaaccggtttgtggcagcagtgaaccagctgggcggtgctgaaaaggccacccccaagggcatcctcaagttcatgggcgtggacgggctcaccatcttccacatcaaaagccacttgcagaagtaccgcctgaacatcaagatgcctggggagggcacccccatgcccggcgacagcgacagcgagtccatcgatgtgggggtgcgccccatgcagcagccacagcagcagatgcagcagccacagcagcagatgcagatgcagcagcagggtgacacgtccatgcgcagcaagtccaggggcgacagcggggatggagtgcggggtggtggcgcagtagttgcacctgcgtcccaggcagctgggccagggttgtcctcctcaacgtcgtctgccatcaaccgcaagaacctggaggaggcgctgctgctgcagatggagctgcagaagaagctacacgagcagctcgagacgcaacgccagctgcaactgagtctggaggcacacgggcgatacatagcaagcttgatggagcaggaggggcttacgcagcgcatcccgcagctgcacggcaatgcacagatgcctgccagcacagccaatgctgcggcagcggtcgcagcggcggctgcggcggcagcagcagcatcaagcaagcacaacgacccacggggagctgcggctgggtccagtgcgtctgggcaaggggggcagtctgcggcaccatctcagcctgcattggagcacagggcagcgtcaggagcaggagccatgcccgcgcactaccagcagcagccgccacagcagcagtacatgcagcagcaacagcagcaacaccctgtgtctgacagcggcatggccgccgccgcggcagccgcagcagccgctgcagctgcagctggcatggcaggccctagcgagatgtacgctgggcactcccagcagcagcaccactcgtcacagcagcagcagcagccatacctccagctgcagcaccggacatcgcagcagcagggcgagcaccagccctccagtatagcagcagccggcagcgcgtggggggaagcagcatccacgggtcacgggcctggg

SEQ ID NO: 62 - DNA, 564 bp, Asteromonas gracilis
  source 1..564: mol_type = other DNA
ggtccatctaagtcgagattacgttggacccccgagttgcacaatcgttttgtacaagcagtcaattatcttggtggtccagatagggcaacaccgaaaggtatcctcaagcttgtcaatggggaaggcctcaccatctaccacatcaaaagccatctacagaagtaccgtttgaacatcaagctaccaatggatccctcgggatcggaatatatgagtgacagccagcaggacgtgtcagcctcaggagagatgcgcagcagtgtagggcatgtggtgaccaggaggagagcttcgccgatgcccggtgctgcccaagaaacgcctcagcaacatgcgacacaagtgcaggcagttggcaacactcaagagggaacatcaaggtgtttagatacttcagccgttgcgtcgggggacaagtccaaaagcctagaggatgcgcttttgtttcagatggagctgcagaagaaattgcatgaacagctggagtcgcagcgccagctgcagcaaagtctggaggcacatggacgttacatcgccagcctgatggagcaggaggggcta

SEQ ID NO: 63 - DNA, 558 bp, Haematococcus pluvialis
  source 1..558: mol_type = other DNA; note = Haematococcus pluvialis-B
aagtcgcgattgcgctggacgcccgagctgcacaacagatttgtgcagtcagtgactacccttgggggcccagaccgagcgacgcccaaaggcatccttaagctgatgggcgtagatggtcttaccatgtaccacattaagagtcacttgcagaagtaccggctcaacattaaaatgccagctgaaagcgggggccaggacagcttgagcgacagccaggaccagcagccacccagcgccatggaagtccgaagcagcagtcgtgggccaacatctacaccacagttgcgggcgccaggctcgagctacgactgtagtgggcaagccccagctctggtctcggcagcgtctgtcaccgctgtacccgcaccgtcttcggccggtgccgcctcttcaggcacaaacaggcgcaaccttgaggatgcactgctgttccagatggagctgcagaaaaagcttcatgagcagttggagtctcaacggcaactacagctcagccttgaggcgcatggtcgctacattgccagccttatggagcaagaaggcttgacacagcgg

SEQ ID NO: 64 - DNA, 333 bp, Chlamydomonas sp.
  source 1..333: mol_type = other DNA; note = Chlamydomonas bilatus-B
gggcaccccgggcaccatgagcagttccgaagacaaagcgaagacaagcctggctcctccaaatcgcggcttaggtggacgcccgagctgcacaaccgcttcgtcaacgctgtgaatcagctgggaggcgcggagaaggccactccgaagggcgtgctcaagctcgtgaacgtcgaggggctcacgatttaccacatcaagagccacctccaaaagtatcgtctcagcatgaagcttcctggcgatgccggcggcgcagagagccctgcagagtctgacagtggcctggagcaggggcaggggcagcagcccacgcggcgccgcagcagcatg

SEQ ID NO: 65 - DNA, 783 bp, unidentified
  source 1..783: mol_type = other DNA; note = Vitreochlamys sp.
ggcatcggacacaacgtgctggcgctccaggggttgccgtctcaatcaccagcccaaatgcttccgttcgtgcccgactacagcggccaattaggagcaggagccttgccgcctggcctccacttacaacaaggcgtgctgaggagcagcagcgccgctcaagcacagagagcccgtctgcgatggacgccagagctgcacaaccgatttgtcactgccgtcaatgcactcggtgacaaagcaactcctaaagggattctcaagcttatgggagctgaggacctgaccatctatcacatcaagagtcacctgcaaaagtaccgcctgaacatccgtcttcccagtggtccccacgccgattctggggtgtcctcagacaatgacatgggcggaatgggcaatgcgctgagcacggtgtcatctgggccgctcggggacctcgcagatttcccacagcagcacgagttccagacgcagcagcaaaggagcttgagcatgtcgcagacccagcagcagctgtcgcagccgcaagcacccttcatacctgggacagcgtccagcccaatgcaggcagcgtctatcgcacccaatccaggaggttcgtcaccgacaatgcagcagatgatgcctgaccagcagcatcgcatgggcaaccttgagcatgcgctgctggtccagttggagctgcagaagaaactgcacgaacaattggaggcccagcgacagatacaaatgaagattgaggcgcatgggcgatatctggcgagtctaatggcctcagag

SEQ ID NO: 66 - DNA, 777 bp, Botryococcus sp.
  source 1..777: mol_type = other DNA; note = Botryococcus terribilis
ggcatggggggccaccaagctcccttgggtggggccccgatcttcacaacaagcagatcagtgagttcctctcagggcatgagagacccgggaaaacagcgtctgcgctggacgccggagctgcacgcgcgcttcgtcggagcggtaaaccagcttcacgggccggaaagggcgacgccaaaaggcattctgaagctgatggatatcgacgggctgacaatctatcacatcaaaagccacctgcagaaataccgcctcaacatcaaactcccgggccaaggcgggccgatgtttgaagaggacgacatgtcggagcggcgcagcacgaagagacggcgcactaaggcgcgctctacgaagcggcggcggaaggcacggggtcgaatctcggacagcgagagcgaggaggaccattacgagagcgaggaagaggagaacgaccctgaggaagtcgaggcggaagaggaggaggacgacgacggaggcgaggtagcgtcccgagggaccggcgctccctcgggcgtccctgtgccggtgccacatcaccctgcaagatcgcagcccatgcctactgccagcagcacagaggactcggtggacgctgacgggaagaaagcaacccgccacgaccggcaggccgcgcttactaaggccctcaacgaccagatggagatgcagcagaagctgatggagcagctggagtctcagcgacgcctccaaagccaagtggagaggcacacggcgtacctgcgggggcttatggaggaggagggcctc

SEQ ID NO: 67 - DNA, 702 bp, Eudorina elegans
  source 1..702: mol_type = other DNA
gggcgtgttgccctccccatggccaaggctgagcgcgcatctggcaccgtagtttgcaacgaggacgactggcttgtggagttttggccggagccagcggcggcagacctcctgagcgcggttccgggcgcaatgcaggcgcagcagcaacacccgcaccaacaactggacccctcccagctatcggagcttctcccacagcagacggggctccagatgggccaatttagcttgcatcaaaccaatgaatacctctcaagcatccagttcgacgcgtttggtggtggcaaagcgactggactggcggggctgggcgggttgctgccagatcaccagcgctcctccacagacggcgcttcggcgctgctgcagtcctcagatttcatgctgcccatggccggcggcctgcagcagccggccttcccggacttagcgttggggggcgtgacgctcaacccaggcatgatgcccgcccacttcctgggtcaccagcaacgagcagcgtcaggacccgccaagagccgactgcgctggacgcccgagctgcacaaccgcttcgtggcgtcggtcaaccaactgggcgggccggagaaggcgacgcccaagggcatcttgaagctcatgagcgtggatggcctcacaatctaccacattaagagccacctccaaaagtaccgcctcaatatccgcctgcccggcgaa

SEQ ID NO: 68 - DNA, 552 bp, Pandorina morum
  source 1..552: mol_type = other DNA
gggctcggcgggctgttgcccgatcaccagcgctcgtccacagatggcgcgtcagcgctgctgcagtcttccgatttcatgcttcccctcggtggcgtgccccaccttatgcagcccggcgtcgctggcttgcagcagtccgcgtttccggacctggcgctgggcggcgtgggcatcaatcagatgctcctgcaaggtcactttctggcgcacccgcagcgagcagcgtcgggccccgccaagagccggctgcggtggacgcccgagttacacaaccgcttcgtggcatctgtcaaccagctgggcggcccggacaaggccacgcccaagggcatcctgaagctcatgggcgtggacggcctcacgatataccacatcaagagccacctgcagaagtacaggctcaacatccggctgcccggggagaccacgcagggcgactctgcggactcggacgcatccgacggcgaggcagcggacccctcggcgtccatggaccgcactgtagagacgcagtcgggactgggcgggggctgcggcggatccttggcc

SEQ ID NO: 69 - DNA, 603 bp, Oedogonium foveolatum
  source 1..603: mol_type = other DNA
gcccgtgcaggcactgtcaaggcacgcctgcgttggacgccagagcttcatacacgctttgtggcatctgtccagagcctgggcgggccagacaaggccactccaaagggcattttaaaactgatgggagttgaaggattaacaatttatcacataaagagccatttgcagaaatttcggcttaatatgcgtctgcctgaatcgacatccaccagccaaggaaacgaggccggcacaagcagtaaacgcagcaagaaagatgagcctcaaggaggcgattctccagctgttgaacaaccgaaagccagtgagacttctacagcttcgcaacctccacccgctgcgttgacaacttccacggcaaccagtgcgcctgcagccgcacttcatcatgaatttcactttccccaacttggccgtggtatcggatctatcacgcgcaaagatttggaggaagccatgttgctgcagatggaaatgcagaaaaaactccacgagcagctggagacacaaaggcacctacaactcagtttagaagcgcatggaaggtacattgcgagtcttatcgagcaggagggcctggcccagcagatgcctgaattg

SEQ ID NO: 70 - DNA, 705 bp, Chlamydomonas sp.
  source 1..705: mol_type = other DNA; strain = M2762
ccgcagcgccctgctcccaaaggctcctccaagtcacgactgcggtggactcccgagcttcataatcgctttgtcaactcagtcaaccagctgggaggaccagacaaggctactcccaagggcatcctcaagttgatgtctgttgatggcttgacaatctatcacatcaagagccacttgcagaagtatcgcctcaacgtgaagaccccgggtgactccgcagcaatgtatgatatggactctgatggggacggtgagggcgaggtgacagacacccgaccggcgcgctccaagggtcagagcgaggcgacaacatcttcgggtgggacagccaggggcaagcacagcaaccggcagcatcaggctgcctcggcgcccgtgggccttcctgcggcagcgccagcgccaccggtgcctggtatgaccaccgccgcgtcgctgcctgtggtgtccagcaacaaccgcaagaacctagaagatgcgctgctcgtgcagatggacctgcagaagaagcttcatgagcagctggagaaccagcgccagctgcaggcccagctgcaggcacacgggcactacatcgccagcctcatgcagcaggagggcatggccacgcccgctgagacgcagcccccagcgccagacaccaagccaccgggcctgcccagcacctccgcaccggctgggctcccgggcccacttccacct

SEQ ID NO: 71 - DNA, 720 bp, Chlamydomonas sp.
  source 1..720: mol_type = other DNA; strain = M2762
ggtgtctccaagtcaaggctgcgttggactccagagctacacaaccgctttgcagctgctgtgaggctgctgggaggaccagacaaggcgacgccaaagggcatcctcagccagatgagcgcgcctgggctgactatttaccacatcaagagccacctgcagaaataccgcctcagcagcaaaagccctggcaacttcagcctgaacgatgactctgacgacgggcttgcaggggagggagacgaggacacgagctgcatggccagtggccaccgccaagactttgccgctgcagcgctgcccggcgatgctgacaggcgagcggcacatcctggctcacccagacgagcgatcgtcaccaacatggaggtgtcaggcagcccggccccgtcagtgcggccgcaagcagcggtcatgacagctccgcgcctgcctgatgccgccgcgagcaatcgcaggaacctggagatggctctgctgaggcagatggagctgcagaagaagctgcatgagcagctcgaggcccagcgccacctgcagctgagcctggaggctcacggccactacatcgccacgctgatgcagaaggagggctacgccgggggcccgacgcccccggagcccgctgcgggagcctgcccggccccagccacagcggcggctggcacggccgtgatcagctcagccgttccccagggcctcgccagggcctgctcatca

SEQ ID NO: 72 - DNA, 585 bp, Chlamydomonas sp.
  source 1..585: mol_type = other DNA; note = Chlamydomonas noctigama
tatcaaatgccaggtattgtcggcgctgcacccacgaagaaaggcagactgcgttggacaccggaattacatgcctgcttcgtcaattcggtccaccagctgggaggatttgagaaagcgacaccgaaagaaatcctgagactaatgaagaccgagggcataacgttgtaccatattaagagccatcttcagaagtacaggcactgcatgaagctcggaagacttgggggcacagacagctcagatgcgtcggaaaacctcccgggcgatcaacagtctccccaaccgatactggattgccatatgcctggacggacagacgggagtttggaggtcgcgccatcgcggccagcggatggaggacgtacgacatgtcatcgtcacaatgacagcacccgacagttttcagatgcaaatgtgcaggcctctgcgtcctcctgcagcattcgccgcaccgcactagaggaagcaattgctctgcagaaggaactgcagaaaaaattccgggagcagatgcagacgcagatagagctgcaagctcgtctggaggcccatggccgctacatagcgacgctggttgagcgt

SEQ ID NO: 73 - DNA, 714 bp, unidentified
  source 1..714: mol_type = other DNA; note = Carteria crucifera
ggaacaccgaagtcccgtttgcgatggactcccgagctgcataaccggtttgtaaacgcagtaaatcagctcggtggcccggagaaagcaacaccgaaaggcattatgaagctcatgagtgtggacggccttacaatataccatataaaaagccacttacaaaagtacagactaaacattcgacttccggctgagtcgcagcttacagatagcagcaccgagaacaaacacgagctgcaaggccagtcgccagttcaagaaccgcaacagcaggagagagattgcggtggaacgtcagcaataccgtgcgaattaacagttcctacgacaacgtcaggaagcggtgcagtaacgaccgttcccaacgccctgtacactaacgtccaagcctccatcgtcgcttcgcaagcctccatcgtcgcttcggcccacccacccccgtcaaccgccgagccaccggttcaggccgggcccagctcatcggagcgcaggtcctcccctgaaccctcttcctcgactaggaaaaatctcgaagaggcgctgcttttccaaatggagctccagaaaaagctgcatgagcagttggagtctcaacggcagttgcagttgagcttagaagctcacggtcgttatattgctagcctgatggagcaggagggtcttacgcataaactaccggaactcacagggcaaacgttaggagcaccttcatca

SEQ ID NO: 74 - DNA, 2238 bp, Volvox aureus
  source 1..2238: mol_type = other DNA; strain = M2242
gggcgtgctgccctccccatggacaaggctgaacgcgcagccagcaacgcgattggcaacgaggacgactggctcttggagttctggccggagccagctgcggcggacttccttggcccagttgcgggcgcgatgcagcagcagcacccgttacaattggaccattcacagctgccagagcaggttccccactctggaagttttcagatgggccagtttggtctcagtcctcctaccagcgattacctcccggggctccagttcgatgcatacggcagcaaaccgcacggtctcagcgggctcggcgggctttttcacgaccaccaacgctcctccaccaacggtgcatcaacactgctccaaccctcagacttattgtttcccatgtgtggggttactcacgcacttctgcaacaccctgggggcgttgcgggcttccaacagcccgcatttccagacctgccgctcggcggggtgggtctgcacccggggttgcttcccggtcactatctgtcgcaccagcaaagagcagcctcttgccccgcgaagagccgcctccgctggacccccgagcttcataatcgcttcgtggcctcagtgaaccagctaggcggccccgagaaggccactccgaagggcatcatgaagctgatgggagtagacggcctcaccatataccacatcaagagccacctgcagaagtatcgcttgaacatacggctgcctggggagacgatgcccggcgacagtgcagacacggatgcctccgacggcgaaggcgaagcaccttcagcgtcaatggacagattggacaggttggaagcaacgcagtcggggatgttggggggagaaggtggcggtggtggcactggaggaggggccactaccgccgcgacggagcaaacggtgtccatcagcgctcagggaaagtctggtcggcgctcgggtcccgccggtggtacttcttgcagcagcggcagtgccccctccgctacgcggcgcaacctcgaggaggcgcttctgttccagatggagcttcagaagaagctccacgagcagttggagacgcaacgtcagctgcagctcagtttggaggctcacggccgctacatcgccagcttgatggaacaggagggccttacggggaaactgccggagctgactgaagccccgctgggtggtggcggcgccagtgcttccattggcagccgtgagcgccgggcttcaggcggcctaggcgcggggctgtcctcagtgccacagccgcccttgggaagcgggccgccactactcaccacgtgtaaggaccgggggggaagagggatagctgccggccgcgctgccagtgggagctgcggcgcgctgcagtcgccagccacgaatctgagtggggcttcaccccatctccaggcttcgtctgggggcgttgccggcgtcgggctacagccgctacagccgccacctgctgctgtgggcgctgcagctgcggcaagtcaatcagcaacagaagcctcagcatcagttccagaacagcaacagcagcaacagcagcaacagcagcaacagcagcaacaacagcaacagaagcaacagcagcaacagaatcaggtgcaagcggttggcaattgcattctcactggtgtacgacatagccctctgcacggattgccatctcttggcggtagtggtggcggcggccgtggaagtgtgaccagtgtgaccagtagtagctcgatgcattttcagatgcagcaggatcatcagcgtttggaactgatgcggttgggccgacttgggtcgcaccccacccctgggtcaccaagcggtaatccgcaggtcgacggtggcggaggaggagcaggagtgagtgagaagcctcagcaagttgttttcaaccctggcctggtggttgtgcgtgagagcagtatcccgctggagcaacctgtagtgatactgcaggatggtggccatcatggccaaacagcagccctagcgcacctgcagcctgagccgcggcaggtgcacccgcagccggcattgaggtcagcatcgggacagctgggatcggggctcgggttgggggatgcattggagggaattatgggcgagagtggaaatggcggtggtggtgccaacggtggcattgtagggccgctgccggactttgactttggagatttcccggatttggatagcggaggattggaacatcagggtttgttgggacctggtgacctg

SEQ ID NO: 75 - DNA, 558 bp, Phacotus lenticularis
  source 1..558: mol_type = other DNA
cccaagtctcgcctcaggtggacgccggagctacacaatcgtttcgtgtcagcagtgaaccagctggggggtgctgacaaggccacacccaagggcattctgaagctgatgggagtggatgggctcactatcttccacatcaagagccacctgcagaagtaccgcctgaacatcaagatgcccggggacagcagtatgcttgcaggcgactctgacagcgagtccatcgacccgcagcgcagcctccgcatgcccgagcccatgcgcagcaagtccaagggcgacagcggggacgcgcagcggggcccagcagtgccgtcgggtgccccagcgccagcagggcccagcatgccggcgccctcgccttctggcgccgggcccagcatgcccgcgccctcgtcgtccacgtcgtctgccatcaaccgcaagaacctggaggaggcgctgctgttccagatggagctgcagaagaagcttcatgagcagcttgagacacaacggcagctgcagctcagtctggaggcgcatggccgctacatagccagcctgatggagcag

SEQ ID NO: 76 - DNA, 747 bp, Stephanosphaera pluvialis
  source 1..747: mol_type = other DNA
ggacaggcgttgatgcagccccaattctcatcgcaagctcccaaacccgacccagctgcgcccaagcagcggctccgatggacacccgagctgcacaatctattcgttcaagcggtggaccagctgggaggcccagagcgtgccacacccaagggcatcctcaatttgatgagtgtcgaaaagctcaccatttaccacatcaaaagtcaccttcagaagtaccgtctcaacatcaagtgccctaatggcgattctggtgcagctggagacagtgacagctacgaccaggcaccatcaggtggcgtggttgaagggcgcagcctcagcaggggctcagtgcccacactcacacacccacatgcttccctgaccagtcttttgaccggcagcatgccttcagcagcagccacttcaccccaggcagtcacaccagcagcatcagcaatccacgtgcattcacccccacctgtgggctgccctacccagccgtctctgcagctcactgtaggcacaagcgcacctggcagcctcagccaggctcctgtgtcccagctcagcaacatgtctggtgggctgtctaccactgtcaccgctgccaacaggaagaacctagaggacgccctgatggtccagatggagctgcaaaagcgcttacatgagcagctggagcaacaacggcagctacagctcagtcttgaggcgcatggcaggtacatagccagcctgatggaacgagagggcatgaca

SEQ ID NO: 77 - DNA, 1758 bp, Chlamydomonas sp.
  source 1..1758: mol_type = other DNA; note = Chlamydomonas eustigma
atggctgaccccatcggccagcccagcctcccaacagacgaccctctgttattaacattgaaaactggaggggtcgacgaacctgacctcgattttaattcttggttagaattctggcctgaatccgaactcccgagcatgcacagctttttacctcaagcaaataatgtgccaatagatgaatcttaccgacaaggatttgccttgcagtcagcaataccagacatcccacggatgcagggcggtctcctagataactatgacagagtaccgaccctcttgtcagctggatcagcgagagacatgcatatgctagcagattttcatggtaatatgaagtctcttccacatcatgctggtttctcttcgttcagtggagacatggggccttctgctgggtcgtattaccatgagaaggagagtagacacggctcaaattcaaggtctcgtcttcgatggacgcctgaactccacaaccgctttgtgaactcagtaaatcagttggcagggcctgagaaagccaccccaaaaggcatcttgaagctcatgaatgtggagggattgacaatctatcacataaaaagccatttgcagaagtatcgcctcaacatcaaaatgccaggagacatgaacttggagagctgtggtgatgactcagacatggatgaaagacccacatccactacgcctgtggacaggaaccgccgagctcctgacctcgagcgccaagtctccctcaatatggacagactggggagagctggcaaggttgctagtgagagggaaactctagatcaaggcaggagaggcatggacacatcatcaggcttggcaggcccagctactggccatacagagtcaacgcctcctcgaccctccacaagtgctggaaccacggctccactctcgtcatcatcatcagcactgaaccgcaaaaatctagaggatgctttgctgttccaaatggagctacagaagaagctgcatgaacaacttgagactcagcgccagctgcagctcagtctagaggcccatgggcggtacattgctagtttgatggagcaggaagttctggcatccaagcaggatggtcaacaaccttctactgagccctcacttggtggaggaggggggacaacccggggtgggatcacagctgctggcttgtgcagacctccttctggggcctctgatgaagtcatgatagctgataaggcagggatggtgtcaacctcaggtgcagcatcaccgcagtacctcgatcatggtggtgcaaagcaaggaggagcccacctgcagtacttcagctccgcaggcagtgaggcctctgcctctgctgcaggcacaggcctgcatggcctttcagtctctgcaagcaatggtgctcatggagccatgctgtcaggtaatcagtttatgggcatgggaggggatagctccatggcgctcatgtctccaggtaagcacatggagactcacatgggtggaaccgtcatgcatgggaagcagctgcatggtgtcatgcgaggaggcatggttggtacagctgcagcacccagtccccccctcctcccgctcatggacgcccatccctcccatgacggctcccctggtgcctccttgttgcctacctccctcatgatggttcagcagtcgtccccagatctggagctattggtacatgaagcaggcagcctggcttcagcgggagactgcaaccatgcatctaagcgcattaagttagaaaatgagttatga

SEQ ID NO: 78 - DNA, 2295 bp, Chlamydomonas sp.
  source 1..2295: mol_type = other DNA; note = Chlamydomonas incerta
atggacaaagctgaacgcgctcctggtggccctaacgccgcaagcgaggacgactggctgctggagttctggccggagcctgccgcggacttccctgcgccggtcgcagcgatgcagccgcagcatcaggacgctacacagctgcaggaggccgttccgcagcagcaagggctcgcgctgggtgcatatggcctcgcccagcagccctctgacttcatgcagtcaagcatgcccggctttgacgcgttcggcagcgggaaggccgcaaccctcgccgggctgcccggcctgctgcccgacgcccagcgcgcctccaccgacggcgcctctgcgcttatgaacgcggcgcagcagtcctcggagtacatgctggccgccggcatgggcggcgtgcagcacttgttagcaccgagcgttggcacggcgctgcccggcagcgggcacaccggcttcgcggacctgtcgatggggggcctggcgggcggccttgcgggcctgggggggccggggatgatgcaccacgggcagttcttcatgcagccgcagcgagcagccacaggccccgccaagagccggctgcgctggacgccggagctgcataatcgcttcgtcaactcggtcaacagcctgggcgggccggacaaggccacgcccaagggcatcctcaagctcatgggcgtggacggcctcaccatctaccacatcaagtcgcacctgcagaagtaccgcctcaacatccggctgccgggcgagagcggcgtggcgggcgactcggcggacggctcggacggcgagcgctcagacggcgagggcggcggcggcggcgggcggcgcgccacgtcgctggagcgggcggacacgatgtcgggtatggccggcgccgtggcgggcgggaggccgggcggggcgctgctgtcgccggggctcgccggcgccacgacgagcaccggtgcggtgggcggcggcggcggcttgatgaccgagcccagcatctcgcggggcgcggtgctgaacgcggccggcgcagctcccgcgggggtggtggcggcggcggtcggcggttcggccggcgtgaagcggccggcggggacgtcgctgagcagcggcagcacggcgtcggcgacgcggcgcaacctggaggaggcgctgctgttccagatggagctgcagaagaagctgcacgaccagctcgagacgcagcggcagctgcagctcagtctggaggcgcacgggcgctatatcgccagcctgatggagcaggagggcctcacctcgcgcctgccagagctcagcggcggcggaccagccgcgccgcccgctggcgcgggcggcgccgccggcggcatgattgcgccgccgccggcgcagcagcagctgcagcaccagccgcagcagctgctgcagccgcagggcagcctgccggccggcggtggctccggcgtagatgctggcagcggcggcggcggcatgaacctgcagccacagcaccagcatgtccacctccatcaccaccagcagcagctgcagccgctcgcgcgccattgcgacacgtgtggcgccggcggcgctggcggggcgcccagcggcggcagcagcatgcagcagctgcaggcggccgagcagcagcgcactgagttggtggcgacggggcggctcgggtccatgcccgcgcccgcgtcttcgtcacccctcgcggggcagcagcaccagccgctggccggcggggcggcgcacgtgcacatgcacgcgcacacgcccggggcgcagccgcagccgcacgtgcagcggcaggactcatatgctggcgcggccgctgcggcggctgcggcggctgctgcagcattgccgcaatcgcattcacacacgctcccagccgacctgtccagcaacgccgtcgctgacccaagcgcagggccgatcaagcctgagcagggcctgtcgccgcaacagcaacagcaacaacaagagcagcaggaagcggagcagctggcgcaggggttgctccatgacagcagcgccggcgcaggggctgtgagcggtagcgacggcggcgggcttggcgacttcgactttggcgatttcggggacctcgacggcggcgcccagggcggcctgcttggcgccggggacctgatcggcatcgcggagctagaggcggcggccgcgcacgagcagcagctgcagcagcaacagcagcagcaacagcagcagcaacagcagcagcaacagctgcagggacaagaacaggagcagctggatgcggaccgcgcaaaacggcagcggctggagcagtag

SEQ ID NO: 79 - DNA, 2226 bp, Chlamydomonas sp.
  source 1..2226: mol_type = other DNA; note = Chlamydomonas schloesseri
atggacaaagctgaacgcgctgctggctgcaccaacgctgcgagcgaggacgactggctgctggagttttggccggagcctgccacggactttccagcgccgggggcagcgatgcagccggcgcatcaggacgcgacacagctgccagagaccattccgcagcagcaagggctggcgctgggcgcatatggcctcacccagcagccggcggactttatgcagtcgggcatacccggctttgacgcattcagcactgggaaggcgcccgggctggccggactgccgagccttcttccggacccgcagcgcgcctccaccgacggggcatcagcgctgatgactgcggcgcagcagccctcagagtacatgctgccgccagccatgggcagcgtgccgcaccttctggcgccaagcgttggcacggtgctgcccggcacggggcacacagggttccccgacctttccatgggcggcatgccgggcggccttgccggcctcggggggcccggcatgatgcatgggcagttcttcatgcagccgcagcgagcagccacgggccccgccaagagccggctacgctggacgcctgagctgcacaaccgcttcgtgacggcggtcaaccagctgggcgggcccgacaaggccacgcccaagggcatcctcaagctcatgggcgtggacggcctcaccatctaccacatcaagtcgcacctgcagaagtaccgcctcaacatacggctgccgggcgagagcgggttggcgggcgactcggcggacggctccgacggcgagcgctccgacggcgagggcggaggaggcgggggcgcgatggggcggcgcgcgagctcgctggagcgggcggacactgcctcgggcatggcggggcccggcgcggtggcgccggggcgggcgggcagcacgccgggagggcagctgctctcaccgggcgtgggggccaccgctggcgccatggcggcggcagccgagccgtccatgtccaggggcgcggtcctgggcgcgtcgggcgctgccgcagccgcggcgccggcggcggccggggcggcggccggcggcgtgaagcgaccggctggggggccgtcgctgagcagcggcagcacggcctcggcggcgcggcgcaacctggaggaggcgctgctgttccaaatggagctgcagaagaagctgcacgagcagctggagacgcagcggcagctgcagctgagcctggaggcgcacggccgctatatcgccagcctgatggagcaggagggcctcacctcgcgactgcccgagctcagcggcggcgcgcccgcggcgccgcccgcggcccccggcggcatgcttgcgcctccgccccagccgcagcaacagcaacagcaacagcagctgctgcagccgcaaggcagcctgccggccggtgggaccaccgccgggttggacgcggctgccgccactggttgtgggggaggcgaaggcccgcaggtgcagcagcagcagcagcagcagcaccatgtccgccactgcgagacgtgcggcgctggtggcgcccccagcggcggtagcagcatgcagcagctgcaggcggcagagcagcagcgcaatgagctggcggctgcggggcggctgggctccatgccggcggccgcgtcctcgtcgccgcacgcggggcacgcgccgctgcagccgccgggcgcacatctgcatgtgcccacgcctgggatgcatcagcagcagcaacagcacatacagccgcaagactcgtacgctggcgcagctgcggcggcagcagcacaagcgtcaccggcggcgctcccacagtcgcattcgcacacgctgccaggggacatgagcagcagcggggtcatggagcagccaggtccagggctggttaaggccgagccaggcctgtcgccgcagcagcagcaacagcaaccgcaaatgcagcaagacgcagatcaggggttactcggcgacggcggcgccggtgcagcgggtgtgagcggcagtgacggcggcggcctaggcgacttcgacttcggggattttggggacctggacggcagcgcccagggtggcctgttgggtcctggcgacctcattggcattgcggagctggaggcagcggccgcacacgaccagcagcaacagctgcagcagctacaccagcaggaggagcaggaacaactggatgcggatcgcgtgaagcggcagcggctggagcagtag

SEQ ID NO: 80 - DNA, 1626 bp, unidentified
  source 1..1626: mol_type = other DNA; note = Chromochloris zofingiensis
atggaccagcgcagggagcccgctgcagctagtcgtgacgctgatacagctgatatcaattggttagagttctggccagaaagcgagtttaaaatcgacggtgctccggcaggtggcagcatggacccgagcttgggcaccttaggaggacttggggattatctgggctctaatctgcagcacccccagctggcaacagctgcgccgctgcaactcaccctgccaggagagtttggcagcgcacagggacttcccttgctaagctctctggaagcgtatcagggaagtggagatctgaatgtgttacaaagcacgcaaccagggcagttgccacagctgctgtcatcagcacccttagctggacattcactgtcgtcatcatatgggtctgacccctcaggtttcactgccaacctcacatctccagcactataccccactgggtcgtatcttgctcaaaattcaaagccaggtttgccgccgaaaacgcgcttgcgatggactcctgagttgcacagcaggtttgtatcagccgtgcatcagcttggtgggccagataaagcaactcccaagggcatccttaagctcataggggtagatgggttgaccatatttcatatcaaaagtcacctgcagaagtacaggctcaacatccgactaccagaatctggacgatcagattcccagggtggttcagagcccctggaaggtggctcaggtgcagacagcaggatgagagcacccagctccacacaaacacaagcgaaactagggttgagcaatcaactagaatcaggtcaagcaggaaccgaagctacaccctcagctacagctgcatcatcgcaggactatgctgccgcccacacagtgtcgcgaccacgcagctcaaatgctggccccagcagcggtgcaggcaaagcagctgttggggatcagctgccagcagctggcggatcaggttcatactcgacggtgggacaaggtggggcaaagccagcagcacagaaacagcagcaattgaagcagcagttgacagctgggttgccaatatctgctgtcacgcgaaaagacctagaggatgcgctcttactacaaatggagctgcagaagaagctgcatgagcaattggagtctcaacgccagctacaactgagtcttgaagcccatggcaggtacatagccagcctgatggaacaggaaggcttggggcacagagtacctgacatagctcagcttactggtgtacagccggagcatttgcagcacaaagagcagcaagcaggcggtgtgcctctgtcattcacccagtcactgacagaagacctcaatgttgatgactcagctttacatatgtttccagggggtcattcagcaggtcctagtcataggcatcagcatgcgacagaacagggtctgccagattctccacaccttcttttgaacttccccgaactcaatgagctagcagatgtcccacaagcaggtgcaggcagcaatatgttgttgccacacatgcccatgggacagcagaatcacatgcagccaacccacaaacggcagcgcttagatgagggtggcagcgcgcagaaccacggcagtcattctggttcacgacaacaacactga

SEQ ID NO: 81 - DNA, 1479 bp, Coccomyxa sp.
  source 1..1479: mol_type = other DNA; note = Coccomyxa subellipsoidea
atgcagcagaaccagcactacaactgggtatcacagcatgtcagcgttgaagatcatcaggagccgcatacgcagcatcaattgccgcaaacagtaggacacgagcacctcacagcagctcacgctgccttctcagactttgggcagcacaggggcgatcaggcgattgcagggatcacaggggatcaggcagaactgttggaggccatatctggtacagagacagaactggggctgccatacactgcccttgatcatctgcagcctcaccacccccagaacgacttgcatcagcatggggttggctatgggatggagagaaatccatcagagcccacacagtcgcaggggaaggatgcgaagccgcgcttgcgctggacgccagagttgcatgcgcgcttcgtgtcagcagtggcttccttggaggggccggacaaggcaacgccgaagagcatcctgaagctgatggcagtggagggcctgaccatctaccacatcaagagccacttgcagaagtaccggctcaacgtgcggctgccgggcgagtccggtgacatgatcagcggccccgacgagtctgaggagccctcgcgacgcaagcggcgcagccgctcgcatggccaggcctccagccgcaggcgcagcagccggcagcgcaaacggcgcaggtcgtcggacgaggacagcgatgaggacgacatggaggatgaggatatggatgacgacgagaacttcgaggagggcatcagccgcgcgcggccgggcaccagcgtgagcggcatcaacggcagcagtccccacggtggcagcccgcgcggtggcagcccccggggggtcagcccccggggccgcagcccgcgaggagggagcccgcatggaggagggaatagcatggcctgcctgcaggtggagccgctggaggtgcctgacctggaccccgagaagcagcacagcctggaggaggccctgctcaagcagatggacatgcagaagcggctgcacgaacagctggaggagcagcggcggctgcagttgtcgctggaggcgcacgggcggtacatcaccagcttgatacagaagaaggggctggaggggcttcctccgcagaccaaggaggctttagatgccgccctggtgcccccacaaggctcagggctgagcacgctgacacacaacacggcaccgcagtggacgccgtcagtgagcgaggggtcttcgctggcgcagcaggtcggccatgtcatgcaccacagcacagccttcatgctgggctcagccagcgccacagaccctgaatcatctctccttctggacaccaatatgcaggcggcggcagctgtgtgggatccgagccaggcgcatgggctggaccagagcggctctaagcagctgtatgaggagccaaaagagctgtacgaggagcccggacagctgtatgaggagaggggcggccacgtcaagccagaggagcaactctga

SEQ ID NO: 82 - DNA, 1551 bp, unidentified
  source 1..1551: mol_type = other DNA; note = Symbiochloris reticulata
atggacacccatgacatagcgcctgcgcttccagagagaagcttggagtggctggaattttggccagaggcagagttcagagcagaggaccacagtatgaacagcaacccgttttgttgggtacagcatgatgatcagcaaccgtcaagtggcgccaaagcctccgctggaccagtgtcagcggcacagacctcggaacctggcatgctctttccagggcaattgcagccggtctctgcagcccttctgagtcacttcacggaggtgcatactggcgtgcaggccgacgtctcgcacagcttcctaccgcccgactatatgcagccccacttctgtggtagtgatttgccggaagctgcccactctatgccgcttgcttcagcagctcagcaggcgcctgtttttggtgcaactgccaacgagccaagctcagctggagctggcagctcacaagctggaaagccgcgcttgagatggacgccagagctgcactcccgctttgtcgctgccgtcaatcacctgggtggacctgatcgagctactcccaaaggcgtgctcaagctgatgttggtggagggtctcactatctaccatatcaagagccacctgcagaagtatcgtctgaacatccgcctgccaggcgactccggccccgtcggcagcctcagtggctcacgcaagaagcgcaaacgcagcagacgagccaggtcctcagacttggaggatgaggaggaggaagatgacatggatgaggcagacagcatggaggacatgttgcctggagatgagctacatggcaggcagcaggcagttggtgaggctggtctggcactggatgcagccctgccagagcagggcaacgctcaggtgcctgggcagcagcctgagcagcagccaaatgctcaacggcaacgagatcttgaagaggccctcatcttgcagatggatatgcagaagcgcttgcacgagcagctggagtcacagcggcagctgcagctcagcctggaggcgcacggccgctacatcagcagcttgatagagcgcgagggtctgcagagcaaactgccagccgggacgcatgcagccatgcagagcggcctgccccggctgcccgaggcgtccctcggcatggctgcaggcatgtgcggtccagcagacggcagcggagcgggcaccattgcgccgggcacgtccggcggcatgtcatggggtcagatgacccatgtcaccctaccgcacagcgcagagtcacctccgctgctgtcccacaccagccgcaccggcgctactgctgcggatgccgggcagttcctgatggtcggggatcccggagatctggggccgctgcccagcatgcttctggacaccgatctgcaagcagcggcagctgtgtgggacgacggcatgcaccggccccgaaagcatgcgccgaatgggcacctagagcatgcttcggggcttgacgagggcctctttgaccagcatgaaggggaagagcatggaaggctgcagcgccgcagacagccatcttctcgcctcagacaatcatga

SEQ ID NO: 83 - DNA, 2409 bp, unidentified
  source 1..2409: mol_type = other DNA; note = Edaphochlamys debaryana
atggccaaggctgaaggtcgccctggaaccattgtgggaagcgaggaggactggttattggagttttggccggagccgacgctggagctctcggggccggccgcgatgcagccgcagcagtcgccgtcgcttgacgcgcccataaccgacctgcagcagcttgcaccacagcaaacatcacaacaggcgcagcagcccggagggctgccgctggggcaatacgcgctcgcctcggctgccgattacctccaaaccgcgcagcatgccctcagcgcatacgacccctaccggaccaagtccgcgccgcccctgcccctgggcctgctcccagaccggccttcggactgtgcatcggggttactgcctccgccagctggcggggaatacctgggggccctgggcacagcccaggggggtctggggcccgtgccacaccccctcatggcgccgggcgctgtctcagggctacagcaagggcagccggggggcggctacggagacctcggcctgggggccatgggcatgggcatgggcggcctgggcctgcagcaggggatgctgcacccgcacgcccattacttcgcggcgcccccccgtgccgccgcgggccctagcaagagccggctccgatggacgcccgagctccacaaccgcttcgtccaagccgtgaacacgctcggcgggcctgacaaggcaacgcccaagggcattctaaagctcatgggcgtggacggactcaccatctatcacatcaagtcccatctgcagaaataccgccttaatattaggctgcctggggactccgccgccgggccccaaggcgactccgccgatgactccgacgcggagggcggcggcggcggcacgaccgcgaccggcatggcggcggcaccgtcgatgtcgttagaccgcggcggcatggagacgacgtcggggctgctgggccgtcggctcggcagcaacgccgccaccgccgccgccgccgcgggcttcctggcggggggcggcgggggtggcggcggcggcatggcggagccaagcctgtccaactcgatagcggcggcgcaggtggcgcagcagcaggccgccgccgcggcggcggcgcagatggcggcggcgcggcccgccggcggcagcaccagcagcggcagtacgccgtcggcgacccggcggaacctggaggaggcgctgctcttccaaatggagctccagaagaagctgcacgagcagctggagacccagcgccagctgcagctcagcctcgaggcgcacgggcgctacatcgcctcgctcatggagcaggagggcctcacctcccgcctgccgcagctcagcagcggcgacgggccgacggcgcagctcgcgctacccgggcccggcggcgagggcggcggcgacgggctgcagcggcagccgtcgggcattggcggcggcggcggcgggccgcagcagggcgggccgctggtgggggcgacggggcaaggcgtggaccacgcggggctaggcggcgtggggcccgacgggcggcggatatcctcccagggcctcggcgcgccctctccccaggcgctcctgccgttccagttgtccagtgctgggcagccgacgggccgccaccagctagggatgcagccctcgccccagcacctgccggggcccggcggcgacggaggcggcggcggggggcccggcgacgagcatcagcggcggcggtccgagatcgcgtacgacggtacgggtgggtcgggcctcactggcggcgccagcggcggctcgtccgtacagcagctggcggtggcggaggcgcagcggcatgacctcatgcgagccggccgcctcgggtccatgccgtccgccgccgccgccgcactccaggccgccggctccaactcgcttccgcagcagcacatgtactcgcccgcgcagcaagactccctcggcctctcccaacagcagcagcagcagcaagcacaagccgacgcccaggcgcacgcgcaagcgcacgcagcggcccaggagcacgcggcggctgccgccgtcgccgccggcatgcagctcagcatggcacacgcgccgtcgggcagcggcctcggcgacggcggcggcgggctgggcgacggcggcggcgggctgggggatttcgacctggcggacttcgtcggcgacctagacgccagcggcgtggcggcgctcgaggggcaggggttcgcggggctccaggggggcctgcagggggactccgagatgggccttcttgcgggtatcggagacgacctggcggcggcggccgccgaggcgcaggcacaagggctcgtctcgccgcggcgcgggtcctcgggcggggaggatagcgggcggagcaagcgcgcgcggctgcagggcagctcgtcgggggagggccagggctag

SEQ ID NO: 84 - DNA, 1824 bp, Enallax sp.
  source 1..1824: mol_type = other DNA; note = Enallax costatus
atggatcccgggcctaatcattctttgggccctttagaaccagatcattgtgaccttggatacctggagttttggcaggaaagcgagttcaagctcgaacctgcagcacaccataccttagggatggaccacctaggcgaccactttttggcagcaggagcagttggagggagcgcagggcagtatgggcagcttggtttggcggcgggaccagacccctacagcaaccaaggcatacctctggtgccagcccttaacgatcagcacttccaggcgggtgacgtatcgctgctgagcacgtccacgggccaaggcaatcaggtgccacagctgctgacaacacctgctctggagtcttatacgtcgtcctacggggcagacccactcagcagcatgccatccggggcaatgctttactcatcgggagcgttcgcgatgcctggcagcaaggggtcttcagcttttgacgctccttcaaacaagacacgtctgcgctggacaccagagttgcacagccgctttgtcagcgcagtgaatcagttgggaggtcctgataaagcaacaccaaaaggcatccttaagctcatgggggtggatgggctaaccatatttcacatcaagagtcacctgcagaagtacaggctcaacatcaggctgccagaaggtgctcagccagccatgagcaccgggtctatgcaggagggggatgcagcagccgcagctgtagatagtgcagctgatacacagacagcggtgatgtccggagcacaagcagctgcagctcaacagccaagtcagcagcagcagcagcgtggtcagcaggataaatctggtcagcaggataagcctactcagcagcagcagcagcaggcaccagctctggtccctcagccaagcagcagtgctggccgtgcagctgcttctctatccccattgatacgtgagggttcaacaacatctatacctgggctcagctcaggggcagtgcccgacatgcaagcaccactgctacctcctggtactggctcgggtgggccggcaggacagcagcaacagcagcagtcgcagcagcaaccgccgccgccgcagcagcagttgaagcaagcgcaacagcagcctctacaacagccgcagcagcatgctcggccggtgccggagacggcagcagcagctggaggagcagcagctactgacgagaacaatgatgctgccatcaagcacagcactcggcgtgacctggagagggctctcctgcgccagatggagctgcagaagcagctgcatgaacaacttgagatgcagcgtgcactgcagcatagctttgaggttcatcagcgctacatacacagcctcatggagcaggaggggctggctcacaagataccagagatgtcagcagccttaggggcagtagcagcagccacagctacagcaccacctggaagtgtggtcagtgaagccatgccagtgcagcccgcacagcccagtaacaacagtcagccactgcagcagcagcagcagcagcagccgccaggagcacaagctggagctgcagtgccagcccctgggcagcagcaacagcagcaggtgctgcagctaccacagaagcaacaacatacaggccatgcaagcgccaatgatgctgcgcctgtggcagctgctgcggtgactgatcagttcctgagtgatgctgagctgttgatgggtttccctgatctgcagcatgacaccggtgacatagaccccatacagcagcacctgctcggggatgaggcagcagggggaccaccaaagaggcagcgcatgtcagggcaggatgtctga

SEQ ID NO: 85 - DNA, 1791 bp, unidentified
  source 1..1791: mol_type = other DNA; note = Mesostigma viride
atgaaccgccccccggtcgggagtgcttcacgtaccgctgaccagcagctgagttcgtcagcggaggtgcagccaaggaccgttcaaaagctgcctaccactgttgacgagcttataaatcaagaatggcctatttggggagagcttgcgcctaacgatgacagcattactacttgctggacggacttgttgaccgggcctccgcccaagaatcaagacatgcaccgtccacaacatgcaaccattcaagatgatacgtctccagggctttaccttgccaggcagcagtatctcccagggatggggacactgccacctggcggggttccccccctgtgtgccccccccggcttgatggatggtggcggcatgaacctggtgccgggcatgcaggcctcgatggcggcggcccagtcgcagcagccgcccaagcagcggttgcggtggacgccagagctgcacgaccgctttgtcaacgcggtgcagaacctggggggagcggaccgtgccactcccaagggtgtcctgcgcgtgatgggtgtgcaggggctgaccatctaccacgtgaagagccacctacagaagtaccggctggccaagttcctgcctgaggagggaggcaactcatccaaatcgctgggcggtagcaagcgcgacacggacagcgacaacgatgacgcgtcggacggcgacccgctcaagatggcggacctcaaggcgggcgcgaccgagctgctgacgggggaggacggctcggtgaacattgaggaggcgctgcgcatgcagatggaggtgcagaagcggctgcatgaacagctcgagctgcaacgcgcgctgcagctcaagatcgaggcacagggtcgctacctgcagcagattatggaggagcagcggaacgccgcgctcgcacgccgcgcgcaggctgggggggcggccagcggcgccaccacgcaggggcaggccacctcggcagggcaagctgcgtccgccagcagtagcaggagcagcgcggggggtgggggaaaggggcccgagggggcagcagcgccggcagcgggtgagggaggtgccggggcggatagcattagccacgctgcggatggtgcccgcgcgggtgcagaggcaggagatgcacagcgccaggcggtcgcctccccatcgggcgcccccgtgtttgcagcctcgggcgtacacgacgcggatggagcgggcgccacctgtccagccgtgggcgcagcaggtggccacgcgccatccccggccctggtgccgaagacagaatcggttgcttgcggtggttcccttgcgatgcccgatgcgctggcgtcactcccagggggcggcggtcaccacctgggcgcgagtggcaagctgccagggtgcgaattgccgctgccctcgtggtcggagccaggtgccgcgctgctgacggccaacgggggcatcttgccgttccctagtaaggtcgaggggcgcaatctaccccagttgtcgctgccgtcgcacctgctgctgggggtggaggatgtggatgatggtggcggtggtgggggtggtggtggcggtgggggccagtccttgcaatcaggtgtcggtgttggcagcaagcgcgcgtacgatgagatgatgggggggggcatcgcgatggaggacggagcgggcgaccggctgcacacggacggcagtgggttgcccacaggggggtccctgctgcccgatgacgcgtccctgctggcggcccagggggggcacgcgtccggtggggcggacccagctccacacatgctctag

SEQ ID NO: 86 - DNA, 1692 bp, unidentified
  source 1..1692: mol_type = other DNA; note = Raphidocelis subcapitata
atggcggagcgcacccccggcagccccgcgggggagggcgacgaggcggttctcgcgggcctcgcgggctggctgaacgatgagctcagctattggccggagtggcccgtcggccccccagccccgcccctggacccccaggcccactgcgacggccccgtgatcgctctcccgggcgctcactgcccgatggagcagcggcaggttgcagcgggaccccccggaccccacgggggggcgggaccccacgcggtggcacagccccagcagcagcatcccgccctgcaggcgggccaggggcacgccttggatgctttccagtcctaccaggcgaccgcttacggcatgcagttggcggtgcacgcgcagcaggggggcttcgaccctggcatgctcggcgcggcgggcgcgctcgcgcccggcgcgctcttcggcgtgccgccagcgtacggcatggccggcggcaagccaggcgccatggccggcggcaacaaatcccgcctgcgctggacgcccgagctgcacgcgtcctttgttgccgccgcggagtcgctcgggggcgctgacaaggcgacgcccaagggcatcctgaagctcatggccgtgcccgggctcaccatcttccacatcaagagccacctccaaaagtaccgcctcaacgtgcgcgcaccggacgggaccgagggcgccagcgacggcggcggggagtcggccgtcgagggcgccagcggcgagggcggcgcgacggtgcgcatgggcgcgctgcgggcggagagcctggatgccacggcgcctagcagcgcgctggcgctgccgccgacggcgctgggcgcttcgccggcggtgggtgtgaagccggagcacccagaggtcgatgcgcacagcctgcttaagcagcagcagcacgcagtgcctgccagcaccaccagcacgtgcgcgggcctcagcagcgcgacgggcctcgaggcggcggcggcggcgggtggcgccggctccgaggcggcggcgggcggcccctccacggcgcggcgcaggaacctggaggatgcgctgcagctgcagatggacctgcagaggcggctgcacgaccagctcgaggcgcagcgggcgctgcagctgagcctggaggcacacgggcgctacatcgcgcggctgatggagcaggagggcctcggccacaggctgcaggacctcgcggcgataaccgcgccgggcccgggcgcgggcgcggaggccgaggcggcgccgggcggcggcgatggcggcggcgcggcgggctctggcggcgcgggctctggcggcgcgggccccggcggcgcgcccgcggcggcgccggccagcgaggcgaacagcagcggcttgagggcggcggccggcggctgcggcggcggcaggagtgtggcgggcggctgctgcgacggtgcgctcccgctggcgcgggcgggctcctctgccctggacagcagcgaccaccccgcagagccgcaccagcagccggcgaggtggcagcagcccacgccgccgccctccgcgtcagggcggcgggacgaccgcagccaagaccagcggctgcacgccgccgccgggcagctgctggcgtggggccgcagcgcgccgccgccgcacgacgcggccgggctggacgcggccggcgcgccgcagggtaagcggccgcggctcagtggcgcctga

SEQ ID NO: 87 - DNA, 1329 bp, unidentified
  source 1..1329: mol_type = other DNA; note = Symbiochloris reticulata Africa
atgctctttccagggcaattgcagccggtctctgcagcccttctgagtcacttcacggaggccgacgtctcgcacagcttcctaccgcccgactatatgcagccccacttctgtggtagtgatttgccggaagctgcccactctatgccgcttgcttcagcagctcagcaggcgcctgtttttggtgcaactgccaacgagccaagctcagctggagctggcagctcacaagctggaaagccgcgcttgagatggacgccagagctgcactcccgctttgtcgctgccgtcaatcacctgggtggacctgatcgagctactcccaaaggcgtgctcaagctgatgttggtggagggtctcactatctaccatatcaagagccacctgcagaagtatcgtctgaacatccgcctgccaggcgactccggccccgtcggcagcctcagtggctcacgcaagaagcgcaaacgcagcagacgagccaggtcctcagacttggaggatgaggaggaggaagatgacatggatgaggcagacagcatggaggacatgttgcctggagatgagctacatggcaggcagcaggcagttggtgaggctggtctggcactggatgcagccctgccagagcagggcaacgctcaggtgcctgggcagcagcctgagcagcagccaaatgctcaacggcaacgagatcttgaagaggccctcatcttgcagatggatatgcagaagcgcttgcacgagcagctggagtcacagcggcagctgcagctcagcctggaggcgcacggccgctacatcagcagcttgatagagcgcgagggtctgcagagcaaactgccagccgggacgcatgcagccatgcagagcggcctgccccggctgcccgaggcgtccctcggcatggctgcaggcatgtgcggtccagcagacggcagcggagcgggcaccattgcgccgggcacgtccggcggcatgtcatggggtcagatgacccatgtcaccctaccgcacagcgcagagtcacctccgctgctgtcccacaccagccgcaccggcgccactgctgcggatgccgggcagttcctgatggtcggggatcccggagatctggggccgctgcccagcatgcttctggacaccgatctgcaagcagcggcagctgtgtgggacgacggcatgcaccggccccgaaagcatgcgccgaatgggcacctagagcatgcttcggggcttgacgagggcctctttgaccagcatgaaggggaagagcatggaaggctgcagcgccgcagacagccatcttctcgcctcagacaatcatga

SEQ ID NO: 88 - DNA, 1905 bp, Tetradesmus sp.
  source 1..1905: mol_type = other DNA; note = Tetradesmus deserticola
atggactctggtgctcatgacttaggggaccatacaggcgattggcttgagttttggcacgagtctgagtttaagttagacggcgtatcgaccgcagcagcccagcccggccagcacgcccctatggacctgcctggagggctcggcgacttcttcttgcccagcggcagcatgctgccgcagccgcactctggagacgcgcagcagttggtattagcacctgcaggcgatccctatgcaggcagcctgaccatgctgccagggctggaacagcaacagcagcactacaaggggcctgacctgtcgttcatgagcacatcctctggagcagcagggcagatgacgcagttgatgccgcctactgcacagctggagtcgtacacttcttcattcagctcagaccctaccctcagcggcatgcattcagcacctatgctgtatcacgcagcttcttttcagctgccgggcacgaggtctgggagcctgcaagaagcccctgcaggcaagacacggctgcgctggacaccggagctacacagccgctttgtgcagtcagtcaattcccttggcggccctgataaggcaacacctaagggcatactgaagctcatgtcagtagatgggctcaccatcttccacatcaagagtcacctgcagaagtaccgtctgaacatcaggctgccggagacctcagagatgggtgcacagcctgcaaacagcagcggatcaccagaccaggaggcaacagcagcaacagacagcgcagcagacacgcacgcaacgctggcgacaagcaccataaacccatcagcagcagcagcagtggcagcaggcgctgccgcacctactgcagctgtggcaccagccagtgcgagtgctggcgggggtagttcgctgcagcagcagcagcagcagcagtcactggttcctacgtctcagcagcagcagcaacagcagccaccgccgccgcagcagcagcagcagcagcgcattttgagtggtgtcgagcagctgtcaggtgcatcgccactgcagctgaccacttcaggcgttctggagatgccagacagcgctgcgtccgcagcccagcagcagcagcagcagcagcagcagcagcaacagcagcccacaggcgctgcagcagatgctgcagaggacagcctgcacatgaagagtgacacgcggcgggacttggaacgtgcactgctgcagcagatgcacctgcagaagaagctgcacgagcagcttgagactcagcgtcagctgcagcatagtctggaggtccaccagcgatacatccacaagctcatggagcaagaagggctagcgcacaagatccctgagatgtcagcagctttcaacgcgggagcattgccaccgccaggcagtgtcgtgagtgaagcaatgccaggccagccactggcagtcggaaccgctcctcagcagcagcagcagcagcagcagcaggcgagcagtgcagcgccgccgctgcagcggcaccattcgctgccgcaccagcaacagttgcacactggtgtcggcaactctgatgcagctgctggtgttggcacctcaaagcgtagcagtagtcaccaccaccaccaccaccatcaacatcatcagcagcaccacccccagcagcagcaccacccgcagccgatgcagcagcagcaggagccaggtcaagacgcagcaggtatcgacccgctgcctggcagctgtggcaacctgctgagtgatcatgagctgctgctggggttccctgagctgcgcgacagtggtgacgagggcgggggcatggggctgctcagcgagcctgggcagccgcaaggcaagcggcagcggctgctgacgcccgacatagccaagtggccttcagtggacagcgctgaaggccagcactga

SEQ ID NO: 89 - DNA, 1428 bp, Tetraselmis sp.
  source 1..1428: mol_type = other DNA; note = Tetraselmis striata
atgaacattcgtcacgacgacgatgccgccgccgccagcgtacaggtgcgagaacgggttccatggtcacaggggattcggggggtggagggtgcggtggtggtggttttcatcctagcccgcttcactccaaccgcgccacagacccgccagaccttgcagcccccgtatcgccagctcgcgcggcatcgtgaggaccctgccacgagccccgacccgcgcaccggtcgtcccgccatggacctcaacgaggacgcggacgcggagctgaactttttcaaggcgatggaagccttcagtccacccagctttgagggcggcgaggcggaggaccacctgcacggcctcagcgtcccgggtctcccccacatggccggggccaccgatacacatgcgcacaatccccccgccagcacaggagacggctcgcaaagcgccacggtgcacaacacatccggggcacgtgggcatgaccttctggtgaacaacgggcacagcatgtgggagccgctgtcgtttgaggaggtgatgcgcaacggcggcgtgaatccctcccaggcctcaagcctcgcgtcgaccagcaccgccgccacggagctgctgatgcatcgcggcaacacgttcctgcccagcgggaatggcggcggcaggcaggcgccgcctggccagtttgggatgggcggcatgccgtccatgatggcgtttggtgccccgcagcagcagcagcaacatcagcaacaccagccgacgcctcagcagcagccgccgcagcggaatgggtcggaggatggcatgcagcactttggaggcctgttcccgcagtcggcggcgttccggccgcggctgcgctggaccaacgacctgcacaaccagtttctggacagcgtcgagcggctgggcggcaccgacaaggccacgccgtccgcgatcctcaagcacatgggcgtggatgggctcagcctgggccacgtgaagagccacctgcagaagtaccgcaccgagctgaagcgcgccaaggcggtgcggggcaaggcgatggacgacatgcaccagatgaagaagggggcgcgcagcaaggcggcggcggcggacgtggcggcggaggcggcggaggtggtggcggaggcgagcggtagcgcggaggcggggctggagcagctgggcgcgacgcagcgggagctgcagcggcagctcgcggcgcgcgcggcgagcgggcccaacgccaaggagctggaggaggcgatgcgcacgcagctggagctgcagaagatgctgtgcgcgcagctggaggcgcagaaggagctgcagcggagcctggagcagcacaccaagtacatttctgtgctgatgaagcggcaatcgggggacgaccttcacgcgcacggcgagggtgacacggccggcgagcatgagatgtccaaggcctga

SEQ ID NO: 90 - DNA, 1611 bp, Trebouxia sp.
  source 1..1611: mol_type = other DNA
atggacaacgacaccatagactggcttgacctggactactggcctgagaaagattccaaaaagcctgcagacatggacaactcgttcgcttggcttgcacagcaagctcagccattatctgggcagccactgccaggatcgcagtaccaggtccagccacatgtaatgcagccacatgatggactgttgtatcacgacacgtttcacccccactccacagcaggatccctgctttcagacctctcaggggatttgctagatacaacagctgtagacatttcgaatgcacagttcgaggccattccttcgcagagcccgcaccagcagagcaacatgcaattacgatcagatactgctcacaacggagccccacaacctctgcaagacatgatccaagctcccgtctttggcagaagcacttcatctatgtctcagcaagcaggcaacaactcccagtctgcagcagcacaagctgctggcaaaccgcgcttgcggtggacgcctgagcttcatactcgctttgttggctgtgtaagtcagctaggcggtcctgaaaaagccacacccaagggtatcatgaagctcatgtcagtggaaggtctcaccatataccatatcaagagtcacttgcaaaagtacaggctaaacatcaggctgccggagtcagagcaggtcgaaatgagtgaagctgtgtcaggtgagcatgaggggcgcaaaagtcagcgaggcaaaaggcgcagcaccaggaaacagcgcaaacgctcaaagcgctcgtctagcaggcgacgtgccttggagaagagtgatggtgatgatgatgaagccgatgacttggatgatgatcagttcgatgaggaggagggtgacaatgagctggatgggcatgctgcttcttctggagtaggggaggcttccagcatgctggatggagtcaccaacagggaagaagatgcacaacgtgaggtgcagcggcagcgcaatctggagcaggctttgctgatccagatggaaatgcagaagaagctgcatgagcagttggaatcacagcggcagcttcagctcagcttagaggcgcatggccgctacatcaccagcctcattgaaagagaaggtctgcagcacagattgttgccgcagctggtagctgcagctgcccccagtctggcacgcactgtccctgccctagcagcactagcagcctcaatgcctccaggctcctcaggtcagatttcagatcagcagactcactatatgcctctgtcagcttctggcgcttcagaattctcccctcagcagctgctggctggcagattttcctccttgcctaactcagtcaatctgaaccaggatcccagtcccggtgcaacagacgctgcaagatccctggacgtgtcgccatcttcgctgagcaggcatgtcagtggtgcagttccgcgtaacccattcggcactatcaatcaggctgcgtttggagagccaagctcgcctggattactgctgaacaccgacctgcaggctgctgctgccgcttgggacgatcagcagcggcatatcctgacaggtcctggaagcagacccttggatgggatgccagctgtacctggtcaatag

SEQ ID NO: 91 - protein, 529 aa, Monoraphidium sp.
  source 1..529: note = Monoraphidium neglectum
MDKAERELRSELREEVGFERNTVWRDMVAMERRTGAVVRQDTHGITDDTIREPWVKRYWQPMTLTVSLIALVTLLLVPIFEDEPEKQNCLALLVFASLLWCTEALPLFVTSMIVPLLVVVLRVLVDRTVSPPERLSPEKAAPAVFHIMFGQVIMLLLGGFAIAAALSKHFIAKQLAVAILSRVGRRPRDVLLANMLVATFASMWISNVAAPVLCFSLVQPILRTLPPTHPFAKALVIGIALASNLGGMTSPISSPQNIFAIERMSIGGDPPSWLTWFAVALPVAFFGNVLCWGLILIVYKPGLKIKEVRPLKPPEDPLSATQIYVVVVSLATVALWCCNNLLSHITGEMGVLAILPLVAFFGFGVLSKDDFNGFLWNVVMLAMGGLALGEAVKSSGLLLTIAQAIQSMVDGLDLWSVLAIFCALVLMATTFISHTVGAMVILPIVQSVGQQMPGHHDKLLVMGAALMCSGAMGLPVSGFPNMNAVALEDPTGVNYVDTVDFLKVGVPGSVMVYWLIVSVGYVLMRAVGY

SEQ ID NO: 92 - protein, 782 aa, unidentified
  source 1..782: note = Entransia fimbriata
MKFSHALKFNSVSEWKAFYIDYVHLKRFVYQLEAAAVTALPETPRLTSLSLVSTLGDVEAGEEASGPSPLVTDAAFVHALERELEKIVTFYAKKEQELVSQIENAETQAREFEARQYTRSRRQGHGQSTMTAPEGGRRVGDEGAVDLREPLLGGKESSGLGSGAGPDSTAQRVWVWQQEDSGVRLAKERLRLQMTDLYVQLFGLQDYIDLNRTGFRKILKKHAKVTHHALQSEFMPRVNVGLDKKHEQQQEAALARVVQLYSIVCCNGSSELARLELKRHLRDHLVWERNTVWQDMVSKERRSASAHVYEEKTAWYLRCLRPLSLLLAVGVLVALLVVPVFPEAPKQNCLALLAFVSLLWCTETIPLYTTSMLVPLLAVVLRVLVDSKTGRRLSPQEAASAIFHTMFSEVIMLLLGGFAIASALSKYFIAKRLATTILSRVGQAPQNVLLASMLVASFLSMWISNVAAPVLCFSLVQPILRTLPSGHPYGRALVMGIALASNLGGMASPISSPQNIFAIEELSLLGDPPSWLQWFVIALPICLVGNIGCWALLLAAYAPHKQPNTIRPLKPTQDPITLSQVYVVVVSVVTVALWCANSWLRQYLGQMGVVAVVPLVAFFGTGILTKDDFNNFLWNVVMLAMGGLALGECVTSSGLLHSIAASIQATVANLGPWQVAAVFCGLVLVATTFISHTVGAMVILPIVMTVGSAMPDPHPKLLVMASVLMCSGAMGLPVSGFPNMNAIALEDGTGKTYLTTLDFLWVGIPSSLLAYGIIITLGYGIM

SEQ ID NO: 93 - protein, 691 aa, Golenkinia sp.
  source 1..691: note = Golenkinia longispicula
MKFTHQLKFNCAPEWKDYYIHYSVLKKLIYQIGGDDIRESAGIGPAEQEPLLPVIDKDEKERQFESLLNTELTRILDFYTRTERELFSQLEGLGLEIREYEEGRLPTTSEGLETELDGRRRFWSSHDLPKDLKAAKDSLAMKLEDLYEELNNLLEYITLNHTGFRKILKKHDKVTQRELKSQYMPLVEAKLVHNKKERVQERIDQVVHQYAVIVCNGHEGRALTELRKKLRDHLVFERQTVWQDMVAIERRTAAAQVSNKAPEGGKGPRKWCQRLWSPLAIGASLLVLALLLRMSLFDEPAKQNCFAMLVFCSMLWCSEAVPLFVTSMMVPFLVVVLRVLTDGTPERQRQTPNQAAGAIFHAMFSQTIMLLLGGFTIAAALSKHFIAKQLAAAILSRVGRKPATVLLTSMMVATFASMWISNVAAPVLCFSLMQTILRTLSPSHPFAKSLVLGIALASNIGGMTSPIASPQNIFAVERMSLHGSPPGWLSWFGVAIPVSILSNLLVWAVILVVYRPGRYIKEVRPLKAPEDPLTGTQMYVIGVSLLTVVLWCCNSFLQKYTGEMGVLALLPMIAFFGFGILNKDDFNGFLWNVVMLAMGGLALGEAVKSSGLLASIAQGIEELVQGMSLYQVSVVFGLMVLVCTTFISHTVGAMVILPIVQSVGERMSDPHPNVLVMSAALLCSCAMGLPV

SEQ ID NO: 94 - protein, 428 aa, Oedogonium cardiacum
EAIPLFVTSMLVPVLAVSLRVIVVDGRRLEPPDAASYLFGKMFSQVVMLLLGGFAIAAALSKHNIARKMAIAVLSRVGREPAHVLLATMMVATFLSMWISNVAAPVLCFSLVQPILRTLDTNHSFAKALVMGIALASNVGGMASPISSPQNIFAIQVMGSGGDKPPSWTQWFAVALPVSVVCNMLIWALLLLVYQPHKHIKEVRPIRAIQDKYTLQQVMVVLVSLVTVALWCLNGVLEPYLGSMGVIAILPLVAFFGFGILTKDDFNAFLWHVVMLAMGGLAVGECVKSSHLLQTIAAEIQEMTVGWSMYAIVCMFCALVLCCTTFISHTVGAFVILPVLKSVGDEMARSGQPNHSKLLVMAAALMCSGAMGLPVSGFPNMNAVALEDQAGLNYVATIDFIKAGLLSSVFAYVIIITLGYSLMFMIGF

SEQ ID NO: 95 - protein, 752 aa, Cylindrocapsa geminella
MKYGTQLKLNMNPDWKDHYINYSMLKHMIYQISNKENQVPIDFEQGEPAEPLQPLLRWNSAPDLESTFVKAFEGELARVIEFYTQKEAELFAKCGTLGLKIHQMDGLSAPPSDSPVASQAEDERIAFWAHVPGHLVAQRDELRKEMEALYVALKDLESFRSLNFEGFRKALKKHDKETTTALMPQLMPVLQSKLSSSQTAIIQQRGDVVLHLFAVVVCHGDLQAARRDLDSRLQDEVLFERRSIWQDMVATERLRGSAPQVVPKDSGSAYGASLTGWFERNKQWTLLLLSFGAFAVLLNYPVFDDESKNNCLAMFVLLSLLWSTEALPLFVTSMLVPLLVVILRVLVDNTQDPPVRLTPQDAAASIFHSMFSQTILLLLGGFTIAAALTKHFLAKQLAVTILSRVGRKPEHVLLANMLVATFLSMWISNVASPVLCFSLVTPILRTLPTHHPFGKALVMGIALASNLGGMTSPISSPQNLFAIERMGLDGHPPSWLAWFLVALPVSFLGNLICWGVLLAVYRPQQKIREVRQLKPSGDPITWKQVYVLVVSLTTVGLWCANTALQQYTGEMGVLAIVPLVAFFGFDILNKDDFNHFLWNVVMLAMGGLALGEAVKSSGLLLTIAEAIKELVIGFDLWTVLVIFCGLVLVGTTFISHTVGAMVILPIVQSVGDQLPGPPHAKLLVMGSALMCSGAMGLPVSGFPNMNAVALEDPTGLNYVATIDFLKVGVVCSFFTYGIIVTLGYGLMLLAGF

SEQ ID NO: 96 - protein, 544 aa, unidentified
  source 1..544: note = Scherffelia dubia
LNVTGFRKILKKHDKVTNKELKGDFLPIVASKLNPKKERVDQVLQELIGVYATIGCEGDVNAAAAQLQVNLRDQVEFERQAVWKDKIEEERKLANAKVVDKGKKAWYADYKKPFCLLLSAALIFGVLGSPLFPSSPPKRNCLAIFVGAAALWCTEAVPLYVTSMLIPAAVVTLRALEDADGVRMTATETADRVFSKMFSQTIMLLLGGFTMAAAITKHLIAKRMAFQVLCRVGRRPGNVLLASMFIALFSSMWISNVASPVMCYGIVQPILRTLAPDDRFASALVMAIALAANVGGMTSPIASPQNIFAVERMAMDGNPPSWLAWFSVSMPVSMFCILVLWRIILAVYRIGPHTAEVRPMRPLDDVFTMQHVFIILISVLTMGLWCANTWLLSVLGGMGITALLPMVAFFGSGILNKLDFESFPWSVVMLAMGGIVLGEAVKSSGLLGFIAQGIVGLVDGFTVWQVLMIFGAVIGVVTSFISHTVGAMVILPVVQSVGEEMAKASGVAHYKLLVMGAALCCSGGMALPVSSFPNMTAASLTDPT

SEQ ID NO: 97 - protein, 499 aa, unidentified
  source 1..499: note = Hafniomonas reticulata
TAGNAEKAEGELSAHLREQVQYERNTVWRDMVANERTKTAESSKQVRTLSQSIVCIAIVAVVALIHANIFPDQPSKNNCLAMLVFVSILWASEAVPLFATSMMVPLLTVLLGIWVDPSQKPPKRLDHTAAASAVFSAMFNQVIMLLLGGFAIAAALSKHFIAKRVAVAILSRVGRKPHNIILASMCVAAFMSMWISNVAAPVLCFSLIAPILRTLDASHPFAKALVMGIALASNVGGMTSPISSPQNLFAIERMSMPPSWLSWFAVALPVAGISIILCWLIILIVYRPWTKVKEVRPLKQDDPITYTQAFVVLVSVVTVGLWCAESQLESVFGQMGVIAILPMLAFFATGILNKDDFNGFLWNVVMLAMGGLVLGGAVKNCGLLEDIANAIKNQVADLELFQVLVVFCLLVLFCTTFISHTVGAIVILPIVQQVGESFPGTPHSKLLVMGAALMCSGAMGLPVSGFPNMNAVALEDATGQTYVQTLDFLKVSVPCSICA

SEQ ID NO: 98 - protein, 234 aa, Tetraselmis sp.
  source 1..234: note = Tetraselmis chui
LLSAAVLWCTEAVPLYVTSMALIFAVVVLRAMLDGDGVRMSAQDTMKRVFSKIFSQTVMLLLGGFTMAAALSKHLIAKRLAIGVMSQVGRKPSNVLIASMAIALFSSMWISNVAAPVLCFSIVQPILRTLPTDDPLGAALVMGIAMSSNIGGMTSPIASPQNIFAIERMSLDGHPPSWITWFAVSMPVSITCMLLVWRLLLAIYPVSSNQDVRPLRQLHDPFTFQHVYIIIVCL

SEQ ID NO: 99 - protein, 434 aa, Volvox globator
LWSLEAIPLFVTSMALPLLIVVLGVLQDSPNTEKPATRLTPQQAATAIFHAMFSQTIMLLLGGFAIAAALSKHAIAKQVAVAILSRVGRKPRNVLLAAMFTATFASMWISNVAAPVLCFGLVQPILRTLDPGHQFAKSLVMGIALASNVGGMTSPISSPQNIFAIERMSLDGQPPSWLSWFTVALPVSIAANMLCWAILLVVYQPDRAIAEVRPIKPNTDPTNGTQVYIIVVALLTVSAWCANTFLQSYTGEMGIIAILPLVAFFGFDVLNKDDFNSFLWNVVMLAMGGLALGEAVKSSGLLAALASDISDLVLGLSLWQVTLIFCGLVLVATTFISHTVGAMVILPIVQSVGERMIGTPHPKLLVMATALTCSGAMGLPVSGFPNMNAVSLEDATGNPIVSTKDFLMVGVPSSIAAYGIIVTLGYGLMLLVGF

SEQ ID NO: 100 - protein, 807 aa, Volvox aureus
  source 1..807: note = Volvox aureus-M1028
MKFTHQLKFNSVPEWREHYIQYAHLKKYIYALAKREADLQAGGQLHDDELLTPLVPETSRQGFSEEGFQRELDAQLASILSFFAVKEAELLAKVSELELDVQSLEKIPNRQEASSMSRISGNPSTTGYHSSSSPRGPVGLPSMSLMSVSPSTLDLARMVDSTPPEDFRKVRVKFWENPPRHVFSPNLNTRRQKLLGRFQDLFIGLHDLREYLHINKEGFRKTIKKHDKLTRSVDLRVRWWPNVEVHLAPVAKQQELERAIAALTDHYAVLYMGGDLTKADEQLSRGLREHITVERNTVWRDMAAMERKYAAVSVKQATAPRDGGRQAHVRWAKLAACCLVFASLLLWGGPVENGQVNQPRNNCLALLVFASLLWSLEAVPLYVTSMALPFLIVAMGVLVDHPSDSKDPPRRLTPQQAAPAIFHAMFSQTIMLLLGGFSIAAALSKHAIAKQVAVAILSRVGRKPRNVLLAAMFTATFASMWISNVAAPVLCFGLIQPILRTLDPGHPFAKSLVMGIALASNVGGMTSPISSPQNIFAIERMSMDGQPPSWLSWFAVAIPVSITCNFLCWALLLLVYQPGRALGEVRPLKPNTDPINGTQVYIIVVSMLTVAAWCANTFLQRYTGEMGIIAIVPLVAFFGFDVLNKDDFNSFLWNVVMLAMGGLSLGEAVKSSGLLAALTNSISELVTGFTMFQVTLIFCGLVLLATTFISHTVGAMVILPIVQSVGESMAGTPHPKLLVMASALMCSGAMGLPVSGFPNMNAVSLEDSTGNAIVSTKDFLYVGVPSSVMAYGIIVSLGYVLMLLVGM

SEQ ID NO: 101 - protein, 606 aa, unidentified
  source 1..606: note = Ignatius tetrasporus
DMYSKLEDLVNFLELNREGFRKALKKHDKVTQRNLSPILLPEALEALNVQDNKNAIEERKQDVVQCYATSQQKGEVLAATNVLKRQQREMVEFERSTVWREHMAVERQHAQATAKAVELTGWQHWWSSHRNLIWIAVAVAAFFIVLLVPMPIFDTVEQHNCAALLTMAGILWCFEALPLFATGMLVPFLVVVLRVMRVADTPQHACTHDCRLSAPDAASAVFHSMMDQVIFLLLGGFTIAAALTKHNIAKQLASAGLASVRDAPGKLLFAAMNIATVSSLFISNVAAPVLCFSLVQPILRTHKHDHPFSKALVIGIALASNIGGMTSPISSPQNIFAIQKMDEDGRAPSWLVWFAVALPVAFACNAACFTVIWHFYKPYKTRTAIRLPKLTDKLNSTQVFVIVVSLLTVGLWCANAQLEKYFGKMGIIAILPVVLFYGSGVLNKDDLNNYLWNVVVLAMGGLALGEAVKSSGLLHVIANLLADAVGSLDLWLVLIVFCGFVLIGTTFISHTVGAMIILPILQTVGKNLPGAPHPQLLVMGAALMCSGAMGLSVSGFPNMTAAAQQASTGEHYINSADFLQVGIPCSVITYGIIVTLGYGLMLAVGL

SEQ ID NO: 102 - protein, 810 aa, Gonium pectorale
MKFTHQLKFNSVPEWREHYIQYAHLKKYIYALAKKEADLQAGAPTIEEGPLAPLLQDARATQGPSEEGFQRELDAQLAALLAFFAVKEADLLAKVSGLELDVQSLEKIPGRREASTLSRLGITGGPCSSSDAIAPGAAVGATVAPLTTLTMDASPSTLDLARMVTCTPPQDHRKVRVKYWENPPRSTFSPNLNSRKMKLQGRFQDLYIGLHDLRQFLIINKEGFRKIIKKHDKLTRMVDLRDCWWPNVEAHLAPTTKQQELDRAIADISDHYAVVYTGGDVAKAEEQLSRGLREHITFERNTVWRDMAAMERKYAAVSVKQAAPPGGAKASRLRDYLQWTKLALSCAVFAILLNVDVWPGPQNGPRNNCLALLVFASLLWSLEAVPLFVTSMAIPFLVVTLEVLTDGTKDPPQRLTPQQAASVVFHAMFSQTVMLLLGGFSIAAALSKHAIAKQVAVTILSRVGRRPRNVLLAAMFTATFASMWISNVAAPVLCFGLVQPILRTLNPGHPFAKALVMGIALASNVGGMTSPISSPQNIFAIERMSMDGHPPSWLSWFAVALPVSIIINLVCWALLLLVYQPERYITEVRRVKPNTDPVNGTQVYIVIVSLLTVVCWCGNSYLQRYTGEMGIIAIVPLVAFFGFGVLNKDDFNSFLWNVVMLAMGGLSLGEAVKSSGLLAALALDISNLVTGLSLWQVAAVFFGMVLVATTFISHTVGAMVILPIVQSVGEAMASPTHPKLLVMGAALMCSGAMGLPVSGFPNMNAVSLEDSTGNAIVSTKDFLLVGVPSSFFAYLVIVTLGYVLMLLVGL

SEQ ID NO: 103 - protein, 801 aa, unidentified
  source 1..801: note = Planophila terrestris
MKFSHSLKFNSVAEWRQHYINYGALKKLSYAIEKQEEEGRQREMFAQQSMDLRQRSMRQSVDLPRGAVEEDEARRRASLDASSSFTGSVQQPLLQRLSSGLGGSLRLSMLERSAEEGGKVSQADFQRSLDSELHKIVDFYITKEAELKKELAAAELDARAAEASSAGSSGFAEMQAERLPRPSFWRTAASDALKAKMHERLCSLYVQLVDLLNFIELNRTGFRKILKKHDKVTDVSLMTDYMPVVTSKLSSKREEDLGAMINEVIKLHAMVMHNSDTNASEVDLKRNLRDHVTYERDTIWRDMVALERRNITVKVPEGAPTGGFAKWWQIYHTPVMVAAALLLFFVILNIDIWPNDTPKRRCAAMLALLVTLWTSEALPLYVTSMLVPLLTVLLRILPDEAAPDGHPQQLPAPKAAEAVFKVMFSQVIMLLLGGFAIAGALSKHYIAKAMASNILSRVGTRPRDVILANMFVATFASMWISNVAAPVLCFSLIQPILRTLPSHHPVSKCLVMGIALASNVGGMTSPISSPQNIFAIELMARDGAAPNWLTWFVVSIPVSIASNLFIWAILLAVYRPGLAIREVRHMRRVQEPITKVQVYVVAVSSLTVALWCASSALEQYLGSQGIIAIFPLVMFFGLGVLDKDDFNNFLWHVVMLAQGGLVLGLAVKGSGLLADVAGAIRGVTAGMSLFGILFTFCALVLVCTTFISHTVGAMVILPIVRSVGQHLSPTPHPRLLVMGAALACSGAMGLPVSGFPNMNAVSLEDATGKTYVNTLDFLMVGLLGSLTTFCVIVTLGYALMM

SEQ ID NO: 104 - protein, 823 aa, Pteromonas angulosa
MKFTHQLKFNSVPEWRDNYIDYAHLKKFIFAIARAEQDDIQQLHGGADGTSMPLLQHTVTMGHDKVDATEDNLRQALDKELQRVISFYMTKEAETLAKVTSMELEINTLEMTRAPRGTSMDHMQGAQRGGSGGSGGSGGGVDLNQQLPSPPQGLATDVEATPPAAHVSASMAAPKSPGTMSRQMRVDFWARANPGARHGGSFGGGSAAFLFVRELQSHKERLRVVFSDLYLQLHDLLNFLRVNKEGFRKIIKKHDKMTSSNLKEHYWPVLESKYPIVRADMLEATINSLVDLYAVIYNQGSVELAKDHLDKLLRDQIKVERNTVWRDMVAQERRTTAAVVEGAVRRPWWAQLTPHIALLSSVLVFAVLLSMEDIFEGEPEKQNCLALLIFVSMLWATEAVPLYVTSLAIPLLVVVLKVLMDKSVDPPVRMTAQQAAPAIFHSMMSQVILLLLGGSAIASALTKHFIAKKLAQVVLSRAGRQPHNVLLALMLVAVVASMFISNVAAPVLCWSLVDPILKAFDAENPFSKSLVMGIALASNIGGMTSPISSPQNIFAIERMSMDGHPPSWLAWFAVALPVSFICILVCWGLILAVYRPWTKVAEVRPLKPSSDKVTFTQFYVVAVTAVTVTLWCFNTQLQPYTGEMGVVATIPIIAFFGFGVLNKDDFLSSPWLVMTLAMGGLGLGEAVKSSGLLLSIAHTIGDVVQGMDVFTVCCIFCALVLVCTSFISHTVGAMIILPIVQSVGEQMPGPHHAKLLVMSSALMCSGAMGLPVSGFPNMFLISKDDGTGKNYINTLDFIKVGVPGSIGAYFVIVSVGYLLMLAV

SEQ ID NO: 105 - protein, 780 aa, Asteromonas gracilis
MKFSHQLKFNSVADWKEHYIHYANLKKIIYEIARLEQARANPDAGEVTELGEPLLSRPPVQNYELAISTKESEFVGELDRELAHIITFILRKEAELVSQLEALDLEVHSLESADPQYRKSLDRDFLDQDAAVGAENGTGYQAGIPARPERIKFWSQGAEPHLAARDARNVAQLKPAQREALSQKFVDLFTTLNDLLEYLVLNREGFRKLIKKHDKMTSSASMKESYWPLIEQRYPEHKRVSMGQNIERLVDLYAILFEGGDTSSAREALSQNLRQHIKVERNTVWRDMVAMERRTVAATVDAPKRKRAWFSTHRKHLSLLLASIVFASMLSLKLFKEPEKSNCAAILVFVSLLWASEAIPLFVTAMVVPVLVVSLRVLVDDSSAKHPIRLSSTDAANAIFHAMFSQVTMLLLGGFTIAAALSKHFIAKQMAVAVLSRVGRLPRNVLLASMCVAAFASMWISNVAAPVLCFSLVQPILRTLDVSSPFAKSLVMGIALASNIGGMTSPISSPQNIFAIERMSMDGVAPSWLSWFAVALPVSFISIILCWLLLLLVYRPGMSTTEVRPLKPYTDPMNMTQVYVIVVSMVTVLLWCANSEVQQFVGNMGVVAVLPMIAFFGFGVLSKDDFNGFLWNVVMLAMGGSALGEAVKSSGLLSTFANDISGQVHGLDLWTVSAIFCGVVLICATFISHTVAAMVILPIVQSVGEAMQENPHPKLLVMATALTCSAAMGLPVSGFPNMNAVSLEDGTGQTFVNTLDFLKVGVPSSVATYFVIISAGYYFM

SEQ ID NO: 106 - protein, 629 aa, Haematococcus pluvialis
  source 1..629: note = Haematococcus pluvialis-B
RVRFWAELGMRRGGRDLRFARDVMRIRFHDLYTSLNDLIEYLSLNREGFRKLIKKHDKLTSTCLKEAYWPDFERRYPMKRKEELERHLDRLIELYAVMFAGGDTRKARDLLLKTLREHIKVERNTVWRDMVALERRTVAATVGAASGVARLSKYKAYSERLGLLAALLVFAALLWAPVFEEKEKSNCLAILVLASMLWATEAIPLFATAMLIPVLVVMLRVLVDHGRPAGAQRLTPQEAAPLIFHAMFSQVIMLLLGGFTIAAALSKHFIAKQMAVAVLSRVGRKPHNVLLASMFVAIFASMWISNVAAPVLCFSLVQPILRTLDVNTPFAKSLVMGIALASNIGGMTSPISSPQNIFAIERMSMDGNPPSWLSWFFVALPVAIISNFICWAAILLVYQPWHKTSEVRPIKPSSDPVTWTQVYVIFVSLATVGLWCGNVALQKYTGEMGVVAVLPMVAFFGFGVLNKDDFNGFLWNVVMLAMGGSALGEAVKSSGLLLTIAQGIQEMVDGLNLWTVTIIFCACVLVCTTFISHTVGAMVILPIVQSVGESMPGQPHPKLLVMSAALMCSGAMGLPVSGFPNMNAVSLEDSTGQTYVGALDFIKVGVPSSILAYAVIITVGYSLMLIIGF

SEQ ID NO: 107 - protein, 380 aa, Chlamydomonas sp.
  source 1..380: note = Chlamydomonas bilatus-B
QTIMLLLGGFAIAGALSKHFIAKQLAIAVLSRVGRKPHNVLLAAMFVATFASMWISNVAAPVLCFSIIMPILKTLDTASSFAKSMVMGIALASNVGGMTSPISSPQNIFAIERMSMDGQPPSWLAWFAVALPVATLCNLLCWLLILAVYQPWRTINDVRPLKPNTDPMNFTQAYVIFISLATVGLWCANTSLQQYTGEMGVVAVLPLVAFFGSGVLSKDDFNGFLWNVVMLAMGGLALGEAVKSSGLLQSMAEGITEVTDGMDLYQVLLVFCLMVLISTTFISHTVGAMVILPIVQSVGEAMPGSPHPKLLVMASALMCSGAMGLPVSGFPNMNAVSLEDSTGQNYVDTLDFLKVGVPGSVLAYGVIVTLGYNLMLMVRF

SEQ ID NO: 108 - protein, 644 aa, unidentified
  source 1..644: note = Vitreochlamys sp.
MKFSSLLKFNCVPEWRDHYVQYGHLKKYIYALAKWEADHLHETQPPDLESLTSPLLPTSGLGSAYGPSEEAFQRELDQSLLEVIRFFSMKEAELVSKCQALLLELVSVEKLPSGSSAGRRSFSGASTPSGAATPTSSAPHGSTANVLAGAKSRLTASPQTSPHVTLSGAKGAGGLGGMHLSPSVVHLMDVANHKDHRTVRVEFWRKPPRRLFQNLEAARSKLKPRLQELYIALHDLAEFLHLNREGLRKVVKKHDKLTRRVTLKTKWWPQVEHLIPPTKKEEVDRAVSELVDNYAVLFTGGSMAAAEQALSQGLRDYVTMERNTVWRDMAAMERRFASLAVKKGSASFIATWWTQPLKIAVSLLVLSVLLNVTIWPEDEKNNCLAVLLFASMMWSLEAIPLFVTSMTIPFLVVCFQLLVDHSQDPPVRMTAQQAAPAIFHAMFSQVIMLLLGGFAIAAALSKHAIAKQISVAVLSRVGRKPRNVLLASMFVATFASMWISNVAAPVLCFGLIQPILRTLDPGHPFAKCLVMGIALASNVGGMTSPISSPQNIFAIERMSVDGKAPSWLSWFAVALPVSIVSNLICWAVLLLVYRPWTKIQEVRPIKPITDPINGTQVYIIVVSVVTVALWCSNTILQPYTGEMG

SEQ ID NO: 109 - protein, 804 aa, Botryococcus sp.
  source 1..804: note = Botryococcus terribilis
MKFSQQIIFNSVPEWKDNYISYAQLKRLIYSEEAARLAAGRDGARGASMRLQRLRKTAMQFKDDLKKEADKVVRFFHEEVESIWSRFHLVLHEIECFEQQEWLPPSAAGLDTSPTSPLLSATSMPATPKTPPMPSPRSSPFQRAGSAGMGLLRTVTGILPRPKRSPRTLSGPLLEVEDGTPRDDSKTWIWQQAEPSIARKRDELRGQLSEVYQDANNMIEFRRLNLDGFRKILKKYDKVLEGLPGAEKLSESQFPGIKERLEALDLTRMQEVEGEVVRLYARVCCSGVYAVAEELLKKQKKDRIVFDRSTVWKEMVERERKRSAAHVEGGAAPRAWYQRHWQLMACAFCGAVFLALLWVPIFEEVEKQHCAALLAFVSLLWCTEALPLFATAMLVPFLVVTLGVLVDRSVDPPHRLTPQEAAPAVFKTMMSQVIMLLLASFAIAGALSKHFIAKWLASVFLSRFGKRPSRVLLANMGVATFASMWISNVAAPVLCFSLLQPILRNLSAKDSFAKALVLGIALASNLGGMTSPIASPQNLFAIQQMSVGGNAPSWLQWWLVALPVAIIGNLVCWGLLLWRYQPPPDDVRELHEAKGFHINPTQMYVVAVSLLTVGLWCCNGYLTPYFGEMGVIAIIPLVAFFGTGVLDKDDFNAFLWNVVILAMGGMALGSAVDSSGLLLTIAKKLEGLVSSHGPWVVLAIFCALVLFATTFVSHTVGAIVILPIVRAVGETMTDPHPKMLVMGAALMCSGAMGLPVSGFPNMNAISLEDKTGVNYLTTKDFLLVGVPSSVATWGIIVSVGYVLM

SEQ ID NO: 110 - protein, 804 aa, Eudorina elegans
MKFTHTLKFNAADSWREHYIQYAHLKKYIYALAKREADLQAGGHVPDDESLHAPLVPETSRSGQGVSEEGFQRELDAQLAAILSFFAVKEAELLAKVSELELDLQSLEKIPNRQEASTMSRLGGGGGAAGSNPTGSPGTAAVAAVSAVLPSLSILSVSPSTLDLARMVNSTPPEEHRKVRVKFWENPPRHVFLPSLHARRTKLQGRFQDLYIGLHDLREYLHINKEGFRKIIKKHDKLTRAVDLRVRWWPNVEAHLAPDAKQQELDRAIAALTDHYAVLYMGGDVAKADEQLSHGLREHITVERNTVWRDMAAMERKYAAVSVKQAAAPGGLRGSYRKLAACCAVFAVMLHVKVWGEDEDEPKNNCLALLAFASLLWSLEAVPLFVTSMALPLLIVVTGVLVGPDKQPLTPQQAAPAIFHAMFSQTIMLLLGGFAIAAALSKHAIAKQVAVAILSRVGRKPRNVLLAAMFTATFASMWISNVAAPVLCFGLIQPILRTLDPGHPFAKSLVMGIALASNVGGMTSPISSPQNIFAIECMSFDGHPPSWLSWFAVALPVSITCNFACWAVLLLVYQPGRAIAEVRPIKPNTDPINGTQVYIIVVSLLTVAAWCANTFLQRYTGEMGVIAILPLVAFFGFDVLNKDDFNSFLWNVVMLAMGGLSLGEAVKSSGLLAALASDISGVVKDLTLFQVAVIFCGMVLVATTFISHTVGAMVILPIVQTVGKAMEGTPHPKLLVMAAALMCSGAMGLPVSGFPNMNAVSLEDSTGNAIVSTQDFLYVGVPSSIIAYGIIVTLGYVLMLLVGL

SEQ ID NO: 111 - protein, 821 aa, Pandorina morum
MKFTHQLKFNSVPEWREHYIQYAHLKKYIYALAKREADLQAGGDEDGLLSPLVPETSRAGQGVSEEGFQRELDAQLASILSFFAVKEAELLAKVSELELDVQSLEKIPSRQEASVSLSRLGAGGGSGGGNPTSSPGSAAVSAVSAVLPSLSLLSVSPSTLDLARMVSSTPPEEHRKVRVKFWENPPRHVFSPNLHARRAKLQGRFQDLYIGLHDLREYLHINKEGFRKIIKKHDKLTRAVDLRARWWPNVEAHLAPDAKQQELDRAIAALTDHYAVLYTGGDVEKAEEQLSRGLREHITVERNTVWRDMAAMERKYAAVSVKQAAAPGLLRFSANRAHVRWAKLALCCVVFAILLNVDFYKENDMEPPDVQRAKNKCLALLVFASMLWSLEAVPLFVTSMALPFLIVMLGVLMDSDGKERLQPKSAAPAIFHAMFSQTVMLLLGGFAIAAALSKHAIAKQVAAAILSRVGRKPRNVLLAAMFTATFASMWISNVAAPVLCFGLIQPILRTLDPGHPFAKSLVMGIALASNVGGMTSPISSPQNIFAIEEMSKGANPPSWLSWFAVALPVSIACNLICWAVLLLAYRPGHVISEVRPIKPNTDPINGTQVYIIVVSLLTVAAWCSNTFLQRYIGEMGVIAIVPLVAFFGFDVLNKDDFNSFLWNVVMLAMGGMSLGEAVKSSGLLSALATDISNLVLHLSIFQITVIFCGMVLVATTFISHTVGAMVILPIVWSVGEKIKGDDPASQSHSKLLVMAAALMCSGAMGLPVSGFPNMNAVSLEDSTGNPIVNTQDFIYVGVPSSIFAYGVIVTLGYVLMSLVGF

SEQ ID NO: 112 - protein, 783 aa, Oedogonium foveolatum
VKFTHYLKFNSVPEWRGQYLAYGLLKKLIYKQEKLLALSRAAPHPESVDIEHEEPTVETPFLQVPSTPPSQLDLSPRRSFDRSFLSGKLSPRSASTTGNPEIEFVRLLGSERTRLNEFIASKYTELTGQLSNVTEVMRVKEMEGGLPHSDPNPYSLAAHRVAFWSQAPMQKAREQLIPQLVELCVFLTGLKDYVEMNKEGFRKILKKWDKVNEARLSEQEMPLVEQTLDVGRRLQDLDEAIGHVMSLYALLTSKGNMDLAWRSMKEHQSEHIRFQRSTVWHDLIALERRTLTATAVRPVDEVMGWWAVNRKHFMIVASLMVFLLLLEAKTFEGDEAAPQRNCLALLVFVSCLWATEAIPLFVTSMLVPLLAVSLRVVVVDGKRLEPPDAATFMFGKMFSQVIMLLLGGFAIAAALSKHNIARKMAISVLSRVGRAPGRVLLATMMVATFLSMWISNVAAPVLCFSLVQPILRTLDTNHQFAKALVMGIALASNVGGMTSPISSPQNIFAIQVMSGGGHSPPSWTQWFVVALPVSAVCNVLIWGLLLAVYQPHKHIKEVRPIRALQDAFTLQQVVVVLVSLLTVTLWCLNGMLEPYLGSMGVIAILPLVAFFGFGILTKDDFNAFLWNVVMLAMGGLAVGECVKSSHLLQSIARGIQDTTAGWSLYCVLAMFCALVLCCTTFISHTVGAFVILPVLQSVGDEMAAAGQPNHSKLLVMAAALMCSGAMGLPVSGFPNMNAVALEDQAGFNYVATIDFIKVGLLSSGFAYVVIISLGYLLMLMVGF

SEQ ID NO: 113 - protein, 188 aa, Chlamydomonas sp.
  source 1..188: note = Chlamydomonas sp.-M2762
PARRLDKLDAAIAKLVDLHAVIYLAGDATKAKDQLSRVLRDVERNTVWRDMVAMERRAVSATVEGTKRPPWWKGYTEHMGLVLSVAVFAVLLSVEIFDEEEKNNCLALLAFVSMLWATEAIPLFATSMLVPPLVVILRVLVDRTKDPPVRLTAQQAAPTIFHAMFSQTIMLLLGGFAIAAALSKHFIA

SEQ ID NO: 114 - protein, 772 aa, Chlamydomonas sp.
  source 1..772: note = Chlamydomonas noctigama
MKFTHQLKFNTVPEWRDHYIHYAALKKIIYAIAKAEADEHQHPAGHDDEHLGVALLDKVEATEEYLIKSLDKELAEVIKFYMAKEAEILGKLEQLDLEVHSLEQRSALGTTLRSTSMPLPSDAVPVILEEDDLSRTESVRASRTEFWRTNSRSLKPTSRALIKDSGKMKQRIIDLYSSLHDLADFLNFNKEGFRKILKKHDKVTSSNLKDRYWRVVEDKYPSKKAEVLEQAMDRLTDQFAVLYLQGDTVKAKDTLGRVLREQIKVERNTVWKDMVAMERRTVAAVIKPGAAEPKKVSFFAKHHSRIMLLLSVVVFASLLSVEIFPEPEKQNCLAMLVFVSLLWATEAIPLYATSMLVPPLVVLLKVLVDRSHEEPIRMTAQQAAPTIFHAMFSQTIMLLLGGFAIAAALSKHFIAKQLAIAVMSRVGRKPHNVLLASMFVATFASMWISNVAAPVLTFSIVMPILKTLETSSAFAKSMVMGIALASNIGGMTSPISSPQNIFAIERMSMDGQPPSWLSWFAVSLPVSSVCIILCWLLILAVYQPWRSVSDVRPLKPNTDPMNMTQVFVIVISMATVGLWCANTALQSYTGEMGVVAMLPLVAFFGFGVLSKDDFNGFLWNVVMLAMGGLALGEAVKSSGLLQSIAEAIKEVTDGYDLYQVLLVFCVMILVCTTFISHTVGAMVILPIVQKVGEDMPGPHPKLLVMAAALMCSGAMGLPVSGFPNMQAVSLEDSTGQNYVDTLDFLKVGVPGSVLAYLVIVSLGYTLMLLVRF

SEQ ID NO: 115 - protein, 209 aa, unidentified
  source 1..209: note = Carteria crucifera
QVYVIVVSVVTVVLWCLNSALQNVTGEMGVIAIIPMVAFFGTGVLSKDDFNGFLWNVVMLAMGGLAMGEAVKSSGLLAAIAEGIKELVAGMDLWEVLAIFCSLILVCTTFISHTVGAMVILPIVQSVGEMALGHPHPRLLVMGSALMCSGAMGLPVSGFPNMNAVALEDSTGVNYVSTVDFLWVGIPSSIFAYVVIVTVGYFLMLMVRF

SEQ ID NO: 116 - protein, 445 aa, Volvox aureus
  source 1..445: note = Volvox aureus-M2242
CLALLVFASLLWSLEAVPLYVTSMALPFLIVAMGVLVDHPNDSKDPPKRLTPQQAAPAIFHAMFSQTIMLLLGGFSISAALSKHAIAKQVAVAILSRVGRKPRNVLLAAMFTATFASMWISNVAAPVLCFGLIQPILRTLDPGHPFAKSLVMGIALASNVGGMTSPISSPQNIFAIERMSMDGQPPSWLSWFAVAIPVSITCNFLCWALLLLVYQPGRALGEVRPLKPNTDPINGTQVYIIVVSMLTVAAWCANTFLQRYTGEMGIIAIVPLVAFFGFDVLNKDDFNSFLWNVVMLAMGGLSLGEAVKSSGLLAALTNSISELVTGFTMFQVTLIFCGLVLLATTFISHTVGAMVILPIVQSVGESMAGTPHPKLLVMASALMCSGAMGLPVSGFPNMNAVSLEDSTGNAIVSTKDFLYVGVPSSVMAYGIIVSLGYVLMLLVGM

SEQ ID NO: 117 - protein, 870 aa, Phacotus lenticularis
MKFTHQLKFNSVPEWRDQYVDYAHLKRFIYAIARAEQDDIQQLHEVHDTTMPLLPHTVTMGHDKVEATEENLRQALDKELQRVISFYMAKEADILAKVTALELGIHALEKLPARGVSLELDPTRQGSQVAAGGVAGGGAPPGGRHVPLLQGAPSVTREGSGGIAHSISPQQSSSSPQLGGPGSGRTAGTGGSQSASPQPSGAMHGGDLEAPLQGGDHTGKTSPHWSRAARVEFWGRAQPAHRFTGGASFSAASFSGPFVRDMQAHKERLRPQFSDLYLSLHDLLGFLRLNKEGFRKIIKKHDKMTSSNLREQYWPLLEAKYPIQRAELLEATIASLVDHYAVIYLGGDVGTSKAHLDKVLRDQIQVERNTVWRDMVAQERRTTAAVVATTYKQKVWAKVTPHIALVSSVAVFALLLSVEDLFPEAPEKQNCLALLIFVSMLWATEAVPLYVTSLAIPLLAVTLRVLVDKTTDPPQRMPAQQAAPAIFHSMCSQVILLLLGGSAIASALTKHFIAKKLAQVVLAQAGRQPHNVLLALMLVATVASMFISNVAAPVLCWSLVEPILKSFDADNPFSKSLVMGIALASNIGGMTSPISSPQNIFAIERMGMDGHPPSWLSWFAVALPVSFICILVCWGLILGVYRPWSKVAELRPLKASADKVTFTQIYVVLVTVVTVGLWCCNTMLQPYTGEMGIVATIPIIAFFGFGVLNKDDFLSSPWLVMTLAMGGLALGEAVKSSGLLLSIAHSIGDLVQDLDLFTVCVIFCGLVLVCASFISHTVGAMIILPIVQSVGEQMPGPHHSKLLVMASALMCSGAMGLPVSGFPNMFLISKDDGTGKNYVNTLDFIKVGVPGSVGAFFVIATVGYVLMLMV

SEQ ID NO: 118 - protein, 632 aa, Stephanosphaera pluvialis
RRQRVRFWASLDTRAELRDLRLVRGMMRYRFNDIYTTLNDLMEYIMLNREGLRKVVKKHDKLTTTVALKESYWPTVDQQLALSKRDAMAQQIEQLVDLYAVMFTAGDVDAAKELLSKNLREHIKVERNTVWRDMVALERRTVAATVQQTTGKAAAKLQRYREPLCLLLSLAAFFALLRAAPFAEPEKNQCLALLALCSLLWATEAVPLFATALAIPPLVVVMRVLVDRSDPAAPHRLTPQQAAPAIFHAMFSQVIMLLLGGFAIAAALSKHFIAKQMAVAVLSRVGRKPHNVLLAAMFVATFASMWISNVAAPVLCFSLVQPILRTMDVTTPFAKSLVMGIALASNIGGMTSPISSPQNIFAIERMGMDGHPPSWLAWFAVALPVAIISNLLAWGLLLLVYRPWTHTTEVRPLKPSSDPINLTQVYVCLVSLATVGLWCANTALQKYTGEMGVVAVLPLVAFFGFGVLNKDDFNGFLWNVVMLAMGGSALGEAVKSSGLLVSIAESIRQLVAGMDLWMVTVVFCLAVLFCTTFISHTVGAMVILPIVQSVGEAMPGPPHSKLLVMASALMCSGAMGLPVSGFPNMNAVSLEDATGQTYVSASDFIAVGVPSSVAAYAVIVTVGYSLMLLVGF

SEQ ID NO: 119 - protein, 813 aa, Chlamydomonas sp.
  source 1..813: note = Chlamydomonas eustigma
MKFTHQIKFNSVPEWRDHYIDYAHLKKIIYAIAKAEADEQQQHHLDEEHPLLTRQQTAHGEKVEATEEALIQALDKELAKIIKFIMAKEAETLGKLAQLDLEVHSLEAQRVGSMFTPPIVNRFTSLQDAGNTRLGGSLPDPQKDGFETLGLADRRPSEVMEEAVRPDLEGGIGSNSFRASRVHFWHSNSLPATTRTGARVLAKDSAKMKPRITDLFVVLHDLKNYLSLNKEGFRKILKKHDKMTSSNLKSRYWCIIEEQYPSKKEEGIMQAINKLVDLYAVLFLKGDFEKASSVLNRVLGEQIKVERNTVWRDMVAMERKTVNAAVHKPQGVATRVTWLQQNMKHILLMLAVLTFATLLTVQTFEEPEKNNCLAMLVFVSMLWATEAIPLFATSMLVPPLVVILRVMVDHTKSPPERMPAKDAAPAIFHSMFSQAIMLLLGGFAIAAALSKHYIAKQLAISVMSRVGRKPQFVILAAMCVAAFVSMFISNVAAPVLTYSIVMPILKTLDTGCPFGKALVMGIALASNVGGMTSPISSPQNIFAIQLMSNDSNPPSWLAWFAISLPVSALCVLMCWSLILIVYQPWRRVAEVRPLKPSTDPINGTQVYVIIISLATVALWCANTVLTPYTGEMGVVAVLPLVAFFGFGVLSKEDFNGFLWNVIMLAMGGMAVGEAVKSSGLLHSIALGIQDLTSGLDLFQVMIIFCLLVLICTTFISHTVGAMVILPIVQSVGESMPGTAHPKLLVMATVLMCSGAMGLPISGFPNMQAVSLDDGMGQNYVSTIDFLMVGVPSSVLAYFVIVSVGYSLMLLVRF

SEQ ID NO: 120 - protein, 809 aa, Chlamydomonas sp.
  source 1..809: note = Chlamydomonas incerta
MKFTHQLKFNSVPEWREHYIQYGHLKKYIYALAKREADLQAGGQEEEALLAPLLLEAGRDQGPTEEGFQQELDAQLAATLSFFAVKEADLLAKVSALELDIQSLEKIPNRAEASTLARMGMGMGGSASPGGPMSSPRAAAAAAMSAVASLVSHSPSTLDLARMVNSTPPEDHRKVRVKFWENPPRHLFSTNLSARRAKLQARFQDLYISLHDLREFLHINKEGFRKIIKKHDKLTRAVDLRARWWPNVEAHLAPAAKQAELDGAIAALTDHYAVLYTRGDVAQAEEQLSRGLREHITVERNTVWRDMAAMERKYAAVSVKQAAAPGARVTWLRTHARWLKLALSVAVLVVLANVEVWPGPENEPRNNCLALLVFASLLWSLEAVPLFVTSMALPLLIVAMGVLVDRSKDPPQRMSPQQAAPAIFHAMFSQTIMLLLGGFSIAAALSKHAIAKQVAVAILSRVGRKPRHVLLAAMFTATFASMWISNVAAPVLCFGLIQPILRTLDPGHPFAKALVMGIALASNVGGMTSPISSPQNIFAIERMSLDGSPPSWLAWFAVALPVAVAANFVCWGLLLLCYQPDKAIAEVRPIKPNTDPINGTQVYIIVVSLLTVAAWCANTFLQRYTGEMGVIAVVPLVAFFGFDVLNKDDFNSFLWNVVMLAMGGLSLGEAVKSSGLLAALALTISDLVTGLSLWQVATIFCGMVLVATTFISHTVGAMVILPIVQSVGEAMPGTPHPKLLVMAAALMCSGAMGLPVSGFPNMNAVSLEDSTGNAIVGTGDFLAVGVPSSVFAYGIIVSLGYLLMLAVGF

SEQ ID NO: 121 - protein, 753 aa, Chlamydomonas sp.
  source 1..753: note = Chlamydomonas schloesseri
MKFTHQLKFNSVPEWREHYIQYGHLKKYIYALAKKEADLQAGGHDDEEALLAPLLEAGRDQGPTEEGFQRELDAQLAATLSFFAVKEADLLAKVSALELDIQSLEKIPNRAEASTLARMGGPGSAMASPGGGGPMASPRAAAAAAMSAVASLVSHSPSTLDLARLVNNTPPEDHRKIRVKFWENPPRHLFSTNLSTRRAKLQARFQDLYISLHDLREFLHINKEGFRKIIKKHDKLTRAVDLRARWWPNVEAHLAPAAKQAELDGAIAQLTDHYAVLYTRGDVAQAEEQLSRGLREHITVERNTVWRDMAAMERKYAAVSVKQAAAPGARVTWLRTHARWLKLAGAVLVFLVLANVQVWPGAENEPRNNCLALLVFASLLWSLEAVPLFVTSMALPLLIVALGVLVDHTKDPPQRMTPQQAAPAIFHAMFSQTIMLLLGGFSIAAALSKHAIAKQVAVAILSRVGRKPRNVLLAAMFTATFASMWISNVAAPVLCFGLIQPILRTLDPGHPFAKALVMGIALASNVGGMTSPISSPQNIFAIERMSLDGRPPSWLAWFAVALPVAVACNFVCWGLLLLCYQPGKAIAEVRPIKPNTDPINGTQVYIIVVSLLTVAAWCANTFLQREVHKSIYATTGEMGVIAVVPLVAFFGFDVLNKDDFNSFLWNVVMLAMGGLSLGEAVKSSGLLAALALSISDLVTGLSLWQVATIFCGMSAAKLWSPPGGRSNQPGPRQQPQKGYCWYNNAGPSGLTNH

SEQ ID NO: 122 - protein, 770 aa, unidentified
  source 1..770: note = Chromochloris zofingiensis
MKFSQTLKFNRRPDWEIHYINYAHLKRLITKVQQAEFAEQNNLPLHFGDEEAGVRSPLLSQTSFNRQQSVSAALTRQQSFTISAAQCDEAFIKALDSELARIIQFYMRKESELLARFESAALRIHSIEGPALPGPAALDTAQRIQFWSQDTKEIALEREKLRSEMTDLYEQLHALSKYLELNFTGFRKILKKHDKMTSQNQYKDSYMPIVEAKLPLKNREMISGVINNLVEMYAVVCTRGDVNRAQAELKRKLKDEVAFERSTVWRDMVAMERRGASVAVHEASSLADQPKKPRWWQAHRQLLLVTLCVTVFAVLLSVPIFQQPEKQNCLALLAFVSLLWCTEAIPLFVTSILVPLLIVVLRVLVDRSADPPRRLPPQEAAPAVFHVMFSQVIMLLLGGFAIAAALSKHFIAKQLAVAILSRVGRKPQYVLLANMLVATFASMWISNVAAPVLCFSLVQPILRTLSPSHAFAKSLVIGIALASNLGGMTSPISSPQNIFAIERMSMDGNPPSWLSWFAVALPVSVLGNLLCWGLILLVYNPGATIKEVRPVKPPEDPLNGTQIYVILVSVATVGLWCFNSFIQHVTGEMGVLAILPLVAFFGFGVLDKDDFNGFLWNVVMLAMGGLALGEAVKSSGLLLTIATGIQDFVAGLGLWSVLAVFCFLVLICTTFISHTVGAMIILPIVQSVGETMSGTPHPKLLVMGSALMCSGAMGLPVSGFPNMNAVALEDPTGQNYVNTIDFLKVGVPGSIMAYGVIVSLGYVLMIAVGM

SEQ ID NO: 123 - protein, 801 aa, Coccomyxa sp.
  source 1..801: note = Coccomyxa subellipsoidea
MKFGAERAGHALLSWLTAAWLWLLQAWEVVAEWGRQCWGALLHAWHYIASAVMQAVHWQTENRIADLGRIPEEVGGDLDRTISLALEEGGDDIKGAFDSELNRITTFHKKKEEELLGAVDKLGEEVSSAVEPSAQQSAPDASSPLLGTSRNAEALYWGQDTVAVRIAREQLRETFQELYVEIQGLIDFVEVNRTGFRKALKKHDKVLGALGHPKMQPTYMPNVEAAFPEKNRLRVSEAQKQLVELYAVVCCHNNLLLAQLELKAQLRSQLKLERTTVWKDMVEKERKENAATVDDSGAESKPWYRSSLFMIALSCVVFAVLLSVPIFEERAKQNCLALLGFASMLWCTEALPLYVTSMLVPLLAVVLRVMVDDSGKHPVRKSAPDAADAIFKAMFSQASSQLFISPHCTIERHVDGLPSYPTTIMLLLGGFAIASAFTKHFIAKRVAVWVLGKVSAKPHAVLIANMFVATFASMWITNVAAPVLCFSVLDPILRTLPSGHSFGKALVLGIALASNLGGMTSPISSPQNIFAIQEMGRDGEPPSWLAWFAVALPVACVGNFACWGFLLLAYRPGRTLKEVRRMPFSSDPFTWKQIYVVVISLGTVGLWCANTALSKFTGQMGIVAIVPMVAFFGFGLLSKDDFNNQLWNVVMLAMGGSALGEAVKSSGLLSSIAHSIEDVVAGMGVWAVFAIFCALVLVATTFISHTVGAMVILPIVSAVGAQMEEPHPRLLVMGAALMCSGAMGLPVSGFPNMTAYAKEDPTGNPWLSTIDFFKVGVPCSLATYGLIVTVGYGIMKFVLGW

SEQ ID NO: 124 - protein, 792 aa, Coccomyxa sp.
  source 1..792: note = Coccomyxa subellipsoidea
MKFGAERAGHALLSWLTAAWLWLLQAWEVVAEWGRQCWGALLHAWHYIASAVMQAVHWVRGLEEVGGDLDRTISLALEEGGDDIKGAFDSELNRITTFHKKKEEELLGAVDKLGEEVSSAVEPSAQQSAPDASSPLLGTSRNAEALYWGQDTVAVRIAREQLRETFQELYVEIQGLIDFVEVNRTGFRKALKKHDKVLGALGHPKMQPTYMPNVEAAFPEKNRLRVSEAQKQLVELYAVVCCHNNLLLAQLELKAQLRSQLKLERTTVWKDMVEKERKENAATVDDSGAESKPWYRSSLFMIALSCVVFAVLLSVPIFEERAKQNCLALLGFASMLWCTEALPLYVTSMLVPLLAVVLRVMVDDSGKHPVRKSAPDAADAIFKAMFSQASSQLFISPHCTIERHVDGLPSYPTTIMLLLGGFAIASAFTKHFIAKRVAVWVLGKVSAKPHAVLIANMFVATFASMWITNVAAPVLCFSVLDPILRTLPSGHSFGKALVLGIALASNLGGMTSPISSPQNIFAIQEMGRDGEPPSWLAWFAVALPVACVGNFACWGFLLLAYRPGRTLKEVRRMPFSSDPFTWKQIYVVVISLGTVGLWCANTALSKFTGQMGIVAIVPMVAFFGFGLLSKDDFNNQLWNVVMLAMGGSALGEAVKSSGLLSSIAHSIEDVVAGMGVWAVFAIFCALVLVATTFISHTVGAMVILPIVSAVGAQMEEPHPRLLVMGAALMCSGAMGLPVSGFPNMTAYAKEDPTGNPWLSTIDFFKVGVPCSLATYGLIVTVGYGIMKFVLGW

SEQ ID NO: 125 - protein, 603 aa, unidentified
  source 1..603: note = Symbiochloris reticulata
MQLGLGRDDMQRLFVLLTGLERYIDLNIAGFRKALKKHDKVLADAESGKLKETYMPTVHRQCCLNKKPILETLYAIVCCDGNNEMALIDLKRRLGETVQFERNTVWKDMVQKDRKRGTLKVDDGLIGSWWHRARQPAAIAMSLAVFVVLLYTPTFREPEKRNCLALLAFTSLLWCTEALPLYVTSMLVPLLVVVLRVLVDGSQHPPQRLSCKQAAPHIFHAMNSQVIMLLLGGFTIAAALSKHAIAKILASWVLSKVGQRPGAVLMANMLVATFASMWISNVAAPVLCFSLVQPVLRTLDATHSFAKSLVMGIALASNLGGMTSPISSPQNLFAIERMSMAGLPPSWLSWFAVALPVAFLGNFLVCGLLLLVYQDPHFTEVRPMQPIKDPINGKQMYIIAVSVGSVTMWCFNSVLQQWFGEMGIIAILPMIAFYGFGILDKDDFNSMLWNVVMLAMGGLALGEAVTSSGLLLSIAEQLQHLVQGASVWRVLVIFCGLVLVATTFVSHTVGAMVVLPIIQSVGSQLSDPHPKLLVMGAALMCSGAMGLPVSGFPNMNAVALEDSKGINYLTTIDFFKVGLLSSLIAYGLIVTLGYGIMYYGIGW

SEQ ID NO: 126 - protein, 813 aa, unidentified
  source 1..813: note = Edaphochlamys debaryana
MKFTHQLKFNSVPEWREHYIQYAHLKKYIYALAKKEADHQADGAGTGDVEGLIAPLLQDGGRASGPTEEGFQRELDSQLAALLGFFAVKEADLLAKVSELELEVQSMEKIPNRNEASNLVRARGGGSAASGTPSPGASPRASAAGAALSALSGLLAASPSTMDLARMVAASPPEDHRSVRVAFWKNPPRHLFSSSLQSRAAKLQSRFQDLYIALHDLREFLHINKEGFRKIIKKHDKLTRSVDLRARWWPNVEAHLAPAAKQAELDGAIAGLTDTYAVVYCRGDASSAEELLSRGLREHITVERNTVWRDMAALERKYAAVSVKQAAGAAKPSWLWRHARWLKLGFALAVFGIMLQYEVWPGPENAPRNGCLALLVFASLLWSLEAVPLFVTSMLLPLLIVLLGVLVDRTKDPPQRMTPQQAAPAIFHAMFSQTIMLLLGGFAIAAALSKHAIAKQFAVAILSRVGRRPRNVLLASMFTATFASMWISNVAAPVLCFGLIQPILRTLDPGHPFAKALVMGIALASNVGGMTSPISSPQNIFAIERMSLDGRPPSWLAWFAVALPVSIACNFVCWGLLLAVYRPERVIAEVRPIKPNTDPINGTQVYICAVSLLTVGAWCANTFLQKFTGEMGVVAVVPLVAFFGFDVLNKDDFNSFLWNVVMLAMGGLCLGEAVKSSGLLAALALGISDLVTGLSLWQVAVVFCGMVLVATTFISHTVGAMVILPIVQSVGEAMPGTPHPKLLVMAAALMCSGAMGLPVSGFPNMNAVSLEDATGNAIVATQDFLLSGVPGSIAAYGIIVTLGHHTMALLAAP

SEQ ID NO: 127 - protein, 762 aa, Enallax sp.
  source 1..762: note = Enallax costatus
MKFTHVLKFNSVPEWRESYINYPLLKKLILAASTAEYHEAYEGLALTQDEEAGPRSPLLSAQPSLSRSLSVTMTREQREKEFLEALDNELAKIIRFYLKKEAEISAKFEELSMMVHHAEGIPSPTPEQMADGHDVTTAARVAFWSQGGRAVAAQREKLKTSLEELYATTFSLANYVEQNRTGFRKILKKHDKLVSHTMSSNYLPIVDQKFPASHAATLHHQLEAITALYAVVCCNGNLEHANSILRKQQQEQVSFQRNSIWKDMVGQERRAATVRVQDGKEVEPESWFTAHRQAVILAIALAVFVVLLTVPIFKQPEKQNCLALLAFASMLWCTEAIPLFVTSMLVPFLVVVLQVLDDVTQEPPERLTPKQAAPRVFHTMFSQTIMLLLGGFAIAAALSKHFIAKQLAVAILSRVGRKPHHVLLANMLVATFASMWISNVAAPVLCFSLVQPILRTLPTTHAFCKSLVIGIALASNLGGMTSPIASPQNIFAVERMGMGGTPPSWLEWFAIALPVSFLGNLLCWGLLLLVYKPGKDIKEVRPLKPTEDPLTGTQIYVIVISLATVTLWCCNSFLQEYTGEMGVLAIFPLVAFFGFGVLNKDDFNGFLWNVVMLAMGGLALGEAVQSSGLLLEISNSISHLVAGQSLWAVLAIFCGLVLVGTTFISHTVGAMVILPIVQAVGQQMPGGDHSKLLVMGAALMCSGAMGLPVSGFPNMNAVALEDPTGANYVYTKDFLLVGVPGSIMAYGIIISVGYLLMLAVGF

SEQ ID NO: 128 - protein, 773 aa, unidentified
  source 1..773: note = Mesostigma viride
MKFGKVLKDDAVPDWIPKYVAYKKLKRVVQRMELTVEQELQQAASKRGAAGSSDVTSPLATKETLLQRKSDEFMEGVEEEVAKVNHFYDEMVSALRCDLEAYEKQLAAQLAGGNKKAFQKMFVLASDLNAYITLNSTAFRKIMKKHDKLTGLHRMDAFVARIKHEGFMEAKALRELSARLEAMMSPDALDSLKQQYHLERQKRSESAGGSTGSPAKPTRILFSIAVFFLILALPPFWSARPASGGNDDGIADVSDGAGVSGGVAFGVDYGYEGEPASLGAQGGVGEAAVAARDRLMRVLWERHYARDEAASSSIGDYVSGNSAFGPTQEERAHRCFALLIFIACMWVLEALPYFVTSLMIPPLVVMLNIMADPTDKDKALSAPDSSRLVLSSMFDHVLILLLGGFTLSAAFGQCAFELRIAGALQRALGHRPWLFMLAIMLLSLFLCMWLSNVTAPVLMLSVLLPILRDFDHGGRYPKALLLGLAFACNLGGMVTPIASPQNAVALVALDAQHFTITFFEWMAVALPFCVLLVVVVWAYLIFALRPDDVVSIPPVMYKTTPLSSKHIWVLLFSLATIGLWSTLSLTVSVLGDLGIIALLFMVFAFGTGVLSKHDLNSFSWHLLLLIAGGNVLGRAVQSSGLIQIVAQIVTPYLHDILWVAALELLAFMIIITTFVSHSVAAIIMMPLIVAIGKEISPLSAEVLVLLCTLADSAAMALPMTSFPNVNSLLVEDDYGVPYLRVVDFIKVGAPVSIMVVTAIATLGYSLAVFVLRP

SEQ ID NO: 129 - protein, 791 aa, unidentified
  source 1..791: note = Raphidocelis subcapitata
MKFTHQLKFNAVPEWKEHYINYPLLKKIIYATRAAECQDAYDGVGGDEEAAGPSASGGSLLRSPRTSLSGGSLRAPLLQGVGGLSLSRSGSVGARAGDSEFIKALDQELARIISFYLRKEGELTSAFESLNLQLHSRDGCDAAAPAAGGAGGGGGGAAGFGTAPAAPAAGAVDGAAAAEAGEAAAAAAVPQSQAERQRRAEFQRRTAYWAANDRGVAAERERFRQKLVGLFVQLDGLKKYLEMNHTGFRKILKKHDKETTQHQYKDSYMAIVDAKLPLRSLEGLNRLIERLREMHAAVCCKGNLEKAERELRSELREEVGFERNTVWRDMVAMERRTGAVVLQEPAHGIADESRQEPWLRRHWQPLALCVSGLAFAALLAAPLFEGAPEKRNCLAMLAFVSLLWCTEALPLFVTSMLVPLLVVVLRVLVDRTVEPPVRLEPQQAAPAIFRVMFGQVIMLLLGGFAIAAALSKHFIAKQLAVAILSRVGRRPRDVLLANMLVATFASMWISNVAAPVLCFSLVQPILRTLPPSHPFAKSLVIGIALASNLGGMTSPISSPQNIFAIERMSMDGHPPSWLAWFAVALPVAFAGNVLCWGLILAVYRPGQKIREVRPLKPPEDPLSPTQVYVVVVSLATVALWCCNSLVAGVTGEMGVLAILPLVAFFGFGVLSKDDFNGFLWNVVMLAMGGLALGEAVKSSGLLLTIAQSVGQQLPGPPHDKLLVMGAALMCSGAMGLPVSGFPNMNAVALEDPTGVNYVDTIDFLKVGVPGSVLAYWIIVTVGYGIMRAVGM

SEQ ID NO: 130 - protein, 731 aa, unidentified
  source 1..731: note = Symbiochloris reticulata Africa
MKFTKELKYNAVEEWRAHYINYAAFKRLIYGEEKRKFGDNERMVPGTPQEDDHPTQEPLLHQTDDKAFMSLLDSELARVHEFYLERERELGGQLDSLLSHARTVEVNERPATPSTEHGRRSSEGRLHLARRSSSRMQGALADLQAEAVSSEFWSQNQDFAVQAAREQLRDDMQRLFVLLTGLERYIDLNIAGFRKALKKHDKVLADAESGKLKETYMPTVHRQCCLNKKPILEGALRKLQTLYAIVCCDGNNEMALIDLKRRLGETVQFERNTVWKDMVQKDRKRGTLKVDDGLIGSWWHRARQPAAIAMSLAVFVVLLYTPTFREPEKRNCLALLAFTSLLWCTEALPLYVTSMLVPLLVVVLRVLVDGSQHPPQRLSCKQAAPHIFHAMNSQVIMLLLGGFTIAAALSKHAIAKILASWVLSKVGQRPGAVLMANMLVATFASMWISNVAAPVLCFSLVQPVLRTLDATHSFAKSLVMVALPVAFLGNFLVCGLLLLVYQDPHFTEVRPMQPIKDPINGKQMYIIAVSVGSVTMWCFNSVLQQWFGEMGIIAILPMIAFYGFGILDKDDFNSMLWNVVMLAMGGLALGEAVTSSGLLLSIAEQLQHLVQGASVWRVLVIFCGLVLVATTFVSHTVGAMVVLPIIQSVGSQLSDPHPKLLVMGAALMCSGAMGLPVSGFPNMNAVALEDSKGINYLTTIDFFKVGLLSSLIAYGLIVTLGYGIMYYGIGW

SEQ ID NO: 131 - protein, 766 aa, Tetradesmus sp.
  source 1..766: note = Tetradesmus deserticola
MKFTHTLKYNSVPEWRESYINYSLLKKLILAASTAEYHEAYEGVHPAADLEDAGPRSPLLSRQASLQASLSRSLSVTMTREQREKEFLETLDNELAKIIRFYLKKEAEITAKYEEVSMMVQHAEGIASPTPGQAAEVSGLQAAQRTAFWSQSSRPVAAQREKLRAALEDLYATCCNLASYVEQNRTGFRKILKKHDKLVSHPMSAIYLPIVDQKFPESHAAHLRAQMDAIASLYSMVCCNGNADKAAAILRKQQQEQVFFERNSIWKDMVGQERRAATLHLQDGKEAVQESWLSTHRQAMLVTLALAVFAFLLYYPIFKEPEKQNCLALLAFASILWCTEAIPLFVTSMLVPFLIVLLRVLDDVDQEPPARLTPQQAAPRVFHTMFSQTIMLLLGGFAIAAALSKHFIAKQLAVAILSRVGRKPHHVLLANMLVATFASMWISNVAAPVLCFSLVQPILRTLPTNHAFCKSLVLGIALASNLGGMTSPISSPQNIFAIERMSMGGSPPSWLQWFAIALPVSFLGNVLCWAVILAVYKPGQNIKEVRPLKPNEDPMSGTQIYTIIVSLATVTAWCCNSFLQAYTGEMGVLAIIPLVAFFGFGVLSKDDFNGFLWNVVMLAMGGLALGEAVQSSGLLATISNLISDLVGGQSLWAVLAIFCALVLVGTTFISHTVGAMVILPIVQSVGDKMPGGHSKLLVMGAALMCSGAMGLPVSGFPNMNAVSLEDSTGQNYIGTADFLKVGVLGSVLAYGIIISIGYGLMLAVGF

SEQ ID NO: 132 - protein, 754 aa, Tetraselmis sp.
  source 1..754: note = Tetraselmis striata
MKFEHALEFNSVPEWRGHYLNYEQLKRLVYAVEAQQSAAQRASLDLSRRPSGVQEDPEAGSPLLPGGSEVEGGQEAEAEFVSCAEGELKRVHAFLTAREAGLLGQWEEAALAAHSAEASYVPARTTRGGAFTRSHWWQQPTMQAQRRTLVATLGSLFVSLHDLSSYAELNETGFRKILKKHDKVTGGALKGALLPVVQARLGAKRARLDQALEEVTSLYATLAFDGDADVAAAHLREGLREQVVFERSAVWKDRMEEERRVATAHVVGPKAAAAKPWLLSGKAIAGLAALALAGAVLGSSAFGADDAGATKRACLAILLASAVLWCTEAVPLYVTSMALIFAVVTLRAMLDGDGARLSAPDAMKRVFSKIFSQTVMLLLGGFTMAAALSKHLIAKRLAIGVMAQVGRRPASVLLAAMGIALFSSMWISNVAAPVLCFSIVAPILRTLPTDDPLGAAMVIGIAMASNIGGMTSPIASPQNIFAIERMSMDGHPPSWLAWFAVSMPVSITCLLLVWRLLLIIYPIDRDQEVRPLRQLDDPFTLHHAFVIAVCLATMGLWCANTWLLHLLGGMGVTALIPMVAFFGFGTLGKDDFESFPWSVVMLAMGGIILGDAATESGLLAAMTEQIVGVVGSLTVCEVLVIFTGVIAVVTSFISHTVGAMVILPVVQSIGAELAKSTGVDHSKLLVMGGALMCSGGMALPVSGFPNMSASSIQDPTGRNYVHVGDFLKTGIPSTAITWLCVIAIGYPIMSAINL

SEQ ID NO: 133 - protein, 261 aa, Trebouxia sp.
MKFSQALKANSVPDWKHHYIHYSRLKKMIFRLEQLQGNAPLSPVPEHRQSLDFTNPSAPLLSRQSSSMLQRTSSGLEHAHIDELMFEREIHDELARVKAFYVEKHDELDAEVLAVLAKVAAAERRGISGPGHQDVEGGQSLPEEQRIAFWTDVNVPRNIKERLSGALTDVYIQLDNLSKFVELNYDGFRKILKKHDKMTNTELSGRLMPTVSDMLAKEQRKGALEGLKNSVVHEYALIAHSGGEREAEQELGRHRRDQLDF

SEQ ID NO: 134 - DNA, 1590 bp, Monoraphidium sp.
  source 1..1590: mol_type = other DNA; note = Monoraphidium neglectum
atggacaaggctgagagggagctgcgcagtgagctgcgggaggaggtcgggttcgagcgcaacacggtgtggcgcgacatggtggccatggagcggcgcacgggggcggtcgtcaggcaggacacccacggcatcacagacgacaccatccgcgagccctgggtgaagcgctactggcagcccatgacgctgactgtgtcgctcatcgcgctcgtcacgctgctgctggtgcccatatttgaggacgagcccgagaagcagaactgcctggccctgctggtgtttgcgagcctgctgtggtgcactgaggcgctgccgctgtttgtgacgtccatgatcgtgccgctgctggtggtggtgctgcgggtgctggtggacaggactgtcagtccgcccgagcgcctgtccccggagaaggccgcccccgcagtgttccacatcatgtttggccaggtcatcatgctgctcctgggcggcttcgccatcgccgcggccctgtcgaagcatttcatagccaagcagctcgccgtcgccatcctgtcgcgcgtcgggcgccgcccccgggacgtgctgcttgccaacatgctggtggcgacgtttgccagcatgtggatctcaaacgtggccgcgccggtgctgtgcttcagcttagtgcagccgatcctgcgcaccctgccgccgacccaccccttcgccaaagccctggtcatcggcatcgccctggcctcaaacctgggcggcatgacatcacccatctcgtccccccagaacatctttgccattgagcgcatgtccatcggcggggaccccccctcctggctcacctggtttgcggtggccctgcccgtggcgttctttggcaacgtgctgtgctgggggctcatcctcatagtctacaagccggggctcaaaatcaaggaggtgcgcccgctgaagcccccggaggaccccctcagcgccacccagatctacgtggtggtggtgtcactggccacggtggccctgtggtgctgcaataacctgctgtcacacatcacgggagagatgggcgtgctcgcaatcctgccgcttgtcgcgttcttcgggttcggtgtgctgtcgaaggacgacttcaacggtttcctgtggaacgtggtgatgctggctatggggggcctggccctgggggaggccgtcaagtccagcgggctgctgctcaccattgcacaggccatccagtcgatggtggacggcctcgacctgtggagcgtcctggccatattctgcgccctcgtcctcatggccaccaccttcatctcccacaccgtcggcgccatggtcatcctgcccatcgtgcagtccgtcggccagcagatgccgggccaccacgacaagctactggtcatgggcgcggccctcatgtgcagcggggccatgggcctgccggtcagcgggttcccaaacatgaacgcggtcgcgctggaggacccgacgggggtcaactacgtcgacaccgtcgactttttgaaggtcggggtgccggggtccgtcatggtttactggctgatcgtgagcgtcggctacgtgctgatgcgggcggtggggtactga

SEQ ID NO: 135 - DNA, 2346 bp, unidentified
  source 1..2346: mol_type = other DNA; note = Entransia fimbriata
atgaagttctcccatgccctgaaattcaattccgtgtcagagtggaaggcattctatatagactatgtacacctcaagaggtttgtgtaccaactggaggccgctgcggtgaccgcccttccagaaaccccgcgtctgactagcttatctctcgtgtccacactgggggatgtcgaggcaggggaggaggcatcgggcccttcgcctttggtcacagatgccgcattcgtgcatgccctggagcgggagcttgagaagatagtcaccttctacgcaaagaaggagcaggagctggtctcacagatcgagaatgcggagacgcaggcgcgggagtttgaggcgcggcagtacaccagaagcaggcgccaggggcacgggcaatccacgatgacggcgccggaaggggggaggcgtgtgggcgatgagggagcggtggatcttcgagaacctctgttgggagggaaggagagcagtggactggggagcggtgccgggcctgactccacagcacagcgcgtgtgggtgtggcagcaggaggacagtggagtgcggctggcgaaggagaggctcaggctccagatgacggacctctatgtccagctgtttggcctgcaagattacatcgacctcaaccgcacagggttccggaagatcctcaagaagcacgccaaggtgacgcaccatgcgctgcagtcggagttcatgccgcgggtcaatgtggggctggacaaaaaacatgagcagcagcaggaggctgctctggcacgtgtggtccagttgtactccatcgtgtgctgcaacggaagctcggagcttgcccggctggagctcaaacgccacctgagggaccacctggtgtgggagaggaacactgtgtggcaggacatggtgtccaaggagcgccggagtgcttcagctcacgtgtacgaggagaagacagcctggtacctgcgctgcctgcggccgctgtcgctgctgctggccgtgggtgtgctggtcgcgcttctcgtggtgcccgtcttccccgaggcccccaagcagaactgtctggcactgcttgcctttgtctctcttctttggtgcactgagaccatccccctctacaccacctccatgctcgtccccctgctggccgtcgtcctccgggtgctggttgactccaagacgggccgccggttgagcccccaggaggcagcctcagccatattccacacgatgttctcagaggtgatcatgctcctcctcggtggctttgccatcgcgtccgccctcagcaagtacttcatcgccaagcgcctcgccacgacgatcctgtcccgcgtcggccaggccccccagaacgtcctcctcgcctcgatgctggtcgcttccttcctttccatgtggatctccaacgtggccgcccccgtgctctgcttctcgctcgtgcagcccatactgcggacgctgcccagtggccacccctacggccgggcactggtcatgggcattgccctcgcatcaaacctcggcggcatggccagccccatcagcagtccccagaacatcttcgctatcgaggagctctcgctgctcggtgaccccccgagctggctgcaatggttcgtcatcgccctccccatctgcctcgtcgggaacatcgggtgctgggccctgctgctcgccgcctacgccccgcacaagcagcccaacacgatccgaccgctgaaaccgacccaggatcccatcacactgtcccaggtctatgttgtggtggtcagtgtggtcacggtggccctctggtgtgccaacagttggctcaggcaatacctgggccagatgggagttgtcgctgtcgtccccctcgtcgccttcttcggcaccgggatactcaccaaggacgatttcaacaacttcttgtggaacgtcgtgatgctggccatggggggcctcgccctcggcgagtgtgtcaccagctccggcctcctccactccatcgccgcctccatccaggcgaccgttgcgaacctgggcccgtggcaagttgcggccgtgttctgcggattggttctggtggccaccaccttcatttcccacacagtcggggccatggtgatactacccattgtgatgaccgtggggtcagccatgccggacccccaccccaagctcctagtcatggcctctgtgctgatgtgctcaggcgccatggggctcccagtgagtggcttcccaaatatgaacgccattgcactcgaagatggcacagggaagacgtacctgaccaccctagatttcctatgggttggcattccaagctcattgctcgcatacgggatcatcatcaccctgggttacgggattatg

SEQ ID NO: 136 - DNA, 2073 bp, Golenkinia sp.
  source 1..2073: mol_type = other DNA; note = Golenkinia longispicula
atgaagtttactcatcagttgaagttcaactgtgccccggagtggaaggactattacattcactactccgtgttgaaaaagctcatatatcagataggaggcgatgatatacgtgagagtgcgggtataggccctgcggagcaagagcctctgctgccagtgattgacaaagacgagaaagagagacagtttgagagtctcttaaacactgagctgacgaggatccttgacttctacacccgcacggagcgtgaactgttttctcagctagagggcctgggccttgagatcagagaatatgaggagggaaggctacccacaacatctgaagggttggaaacagagctggatggtcgccggcgcttctggtcgtcccacgacctccccaaggacctcaaagccgccaaagacagcctggccatgaagcttgaggacctgtacgaggagctgaacaatctgttggagtacatcacgttgaaccacacgggcttcaggaagatcctcaagaaacacgacaaggtgacccagagggagctgaagtcgcagtacatgcccctggtggaggccaagctggtccataacaagaaggagagagtgcaggagcgcatcgaccaggtggtccatcagtatgcagtgattgtctgcaatggccatgaaggccgggccctcacagaactcaggaagaagttgagggaccaccttgtattcgagcgccagactgtgtggcaggacatggtggcaatagagcgtcgcacagctgcagcacaggtcagcaacaaggcgcctgagggaggcaaaggcccccgtaagtggtgccagcggttgtggagtcccctggccataggtgcctctttgctggtgctggctctgctgctgaggatgtccctctttgacgagccggcgaaacagaactgttttgctatgttggtgttctgctccatgttgtggtgctcagaggcggtgcccctgtttgtgacgtccatgatggtgcccttcctggtggtggtgttgagggtgctgacagatggcactcctgagcggcagaggcagacccccaaccaggcagccggggccatattccatgcgatgttctcacagaccatcatgctgctcctcggtggcttcaccattgcggcggctcttagtaagcacttcatagctaaacagttggcagcggcaattctcagccgagtgggtaggaagcccgcaacagtgctgctgaccagtatgatggtggcgacctttgccagcatgtggatttccaacgtggcggctccggtgctgtgcttttctctgatgcagaccatcctgaggaccctttccccctcacacccgtttgccaagagtttggtgctgggcattgccctggcctccaacatcggggggatgacctcgcccattgccagcccacagaacatcttcgcagtagagaggatgtcactccatggcagccccccggggtggttgagttggtttggtgtcgccattcctgtcagtatcttgtccaacctgcttgtttgggccgttatcttggtcgtgtacaggccaggccgctacatcaaggaggtaaggcccctcaaggcccccgaagaccccctcacaggtacccaaatgtacgtcatcggggtcagtctgctcactgtggtgctctggtgctgcaactccttcctccagaaatataccggagagatgggggtgctggccctactgcccatgattgccttctttggctttggtatcttgaacaaggacgacttcaacggctttctttggaacgtggtgatgttggccatggggggtctggccttaggcgaggcagtgaagagcagcggtctgctggcctccattgctcagggcattgaggagctggtgcagggtatgagcttgtaccaggtgtctgtggtgtttggcctcatggtgctggtgtgtaccaccttcatcagccatacggtgggggccatggtaatcctgcccattgtgcagtccgtgggggagaggatgtcagatccccatcccaacgtattagtcatgagcgctgcactgttgtgtagctgcgcgatgggtctgccggtc

SEQ ID NO: 137 - DNA, 1284 bp, Oedogonium cardiacum
  source 1..1284: mol_type = other DNA
gaggcaatccccctctttgtgaccagcatgctcgtgcccgtgttggccgtatccctgcgggtgatagtggtggacggaaggaggctggagccgcctgacgcggcctcatacttgttcgggaagatgttctcacaggtggtcatgttgctgctcggtggttttgccattgctgcagctttgagcaagcataacattgcgcgcaagatggccatagccgtcctttcaagagttgggcgcgaacctgcacacgttttgctagccacaatgatggttgccactttcctatctatgtggatttcaaatgtggctgcaccagtgctttgtttctctctcgtgcagcccatcttgaggacactggatacaaaccatagctttgcgaaggcgctggtgatgggcatcgcgctagcttccaatgtgggcggcatggccagccccatcagtagtccccaaaacatattcgccatccaagtcatgggcagtgggggtgacaaacctccaagttggacgcaatggtttgctgtggcattgccagtgtccgtcgtgtgcaacatgctcatttgggcactgctattgctggtgtaccaaccacataagcacatcaaagaggttcgccccattcgtgcaatccaggacaagtacactcttcagcaagtcatggtggttctggtcagtctggttacagtagctctgtggtgcctgaatggcgtgctggagccctacctgggatctatgggtgtcatagctatcttaccgctggtggcgttcttcggttttggcatcttgaccaaggatgatttcaacgcgtttttgtggcatgtggtcatgttggccatggggggactggcagtgggagagtgtgtgaagagctcacacttattgcagaccattgctgccgaaatccaggagatgacggttggatggtccatgtatgctattgtgtgcatgttttgtgcgctggtgctatgctgcacgaccttcatttcgcacacggtgggcgccttcgtgattttgcctgttttgaagagtgtgggagacgagatggcgagatccgggcagccaaaccactccaagcttttggtcatggccgcagctctgatgtgctctggtgccatgggcctgccagtgagtggtttccccaacatgaacgcggtggccctggaggaccaagcggggctcaactacgtagccacaatagacttcatcaaagcagggctgctcagctcagtgtttgcatacgtcattattataaccctgggatactcacttatgtttatgatcggtttt

SEQ ID NO: 138 - DNA, 2256 bp, Cylindrocapsa geminella
  source 1..2256: mol_type = other DNA
atgaagtacggcacgcagcttaagctgaacatgaatcctgactggaaggatcactacatcaactattccatgctgaagcacatgatctatcagatctccaataaggagaaccaggtgcctattgactttgagcagggagagccagcagagcccctacagcccctccttcgatggaacagcgcgccggacctggagagcacgttcgtaaaggcatttgaaggggagctcgcgcgtgtgattgagttctacacgcagaaagaggcagagctcttcgccaagtgcggtactctaggtttgaagattcaccagatggatgggctgagcgccccgccgtcggactctccagtcgcttctcaggccgaggacgagcgcatcgcgttctgggcgcacgtgccgggccacttggtcgcccagcgcgacgagcttcgcaaagagatggaggcgctgtacgtggcccttaaggacctggaaagcttcaggtcgctgaacttcgagggcttccgcaaggctctgaaaaagcacgataaggagaccaccactgcgcttatgccgcagctgatgcccgtgctgcagtccaagctgtcctcgtcgcaaacggccatcatccaacagcgcggagacgtggtgctgcacctgttcgctgtggttgtgtgccatggcgacctgcaggcggctcgcagggacctcgactcgcggctgcaggatgaggttctgttcgagcgacgcagcatctggcaggatatggtggcgaccgagcggctgcgtggctcagcgccgcaagtggtgcccaaggactccggatcggcgtacggcgcgagcctgacaggctggttcgagcggaacaagcagtggacactgctgctgctgtcgttcggggcgttcgcggtgctgctgaactaccccgtgttcgacgacgagtccaagaacaactgcctcgcgatgttcgtgctgctgtctcttctgtggagcacagaggcgctcccactattcgtcacgagcatgctggtgccgctgctggtggtcatactcagggtgctggtggacaacacccaggacccgcccgtgcggctgacgcctcaggacgctgcggcgtccatcttccactcgatgttctcgcaaaccatcctgctgctgctcggcggcttcaccatcgctgccgcgctgaccaagcacttcctggccaagcagctggcggtgaccatcctctcacgtgtcggccgcaaacccgagcacgtgctgctggccaacatgctggtagcaacgttcttgtcaatgtggatatcgaacgtggcgtcgcctgtgctgtgcttctcgctggtcactccgatcctgcgcacgctgcccacgcaccaccccttcggcaaggcgctcgttatggggatcgcgctcgcgtcgaacctcggggggatgaccagcccgatcagcagccctcagaacctgtttgcgatcgagcgcatgggtctggatggtcacccgcccagctggctggcgtggtttctggtggcactgcccgtgtccttcttaggcaacctgatctgctggggcgtgctgctggcagtgtaccgcccgcagcagaagattcgagaggtccgtcagctgaagcccagcggcgaccccatcacgtggaagcaggtgtacgtgcttgtcgtgagcctcaccacagtgggcctgtggtgcgctaatacggccttgcagcagtacacgggcgagatgggcgtgctggcgatcgtgccgctggtggcgttcttcggtttcgacatcctcaacaaggacgacttcaaccactttctgtggaacgtggtgatgctggcgatgggcggcctggcgctgggcgaggcagtcaagtcgtccggcctgctgctgaccatcgccgaggcgatcaaggaactcgtaatcgggttcgatctctggactgttctcgtgatcttctgcggccttgttctcgtgggcaccaccttcatctcgcacaccgtcggcgcgatggtgatcctcccgatcgtgcaatccgtgggcgaccagctgcccggcccgccgcacgcgaagcttctcgtgatgggttcggccctaatgtgctccggcgcaatgggcctgcccgtgagcggcttcccgaacatgaacgcggtggcactggaggatcccacggggctgaactacgtggccaccatcgacttcctcaaggtcggcgtggtctgctcgttctttacgtacggcatcatagtaacgctcggctacggcctcatgctgctggccggcttc

SEQ ID NO: 139 - DNA, 1632 bp, unidentified
  source 1..1632: mol_type = other DNA; note = Scherffelia dubia
ctgaacgtgacggggttccgcaagatcctgaagaagcacgacaaggtgacgaacaaggagctcaagggcgacttcctgcccatcgtggccagcaagctcaaccccaagaaggagcgcgtggaccaggtgctgcaggagctcatcggcgtgtacgcgacgataggctgcgagggcgatgtgaacgccgcagccgcgcagctgcaggtcaacctgcgcgaccaggtggagttcgagcggcaggcggtgtggaaggacaagatcgaggaggagcgcaagctggccaacgccaaggtggtggacaagggcaagaaggcatggtacgcggactacaagaagcccttctgcctgctgctcagcgccgcgctcatcttcggggtgctcggctcgcccctcttcccctcctccccccccaagcgcaactgcctcgccatcttcgtgggcgccgccgcgctgtggtgcaccgaagcggtgccgctgtacgtcacctccatgctcatccccgccgccgtcgtcacgctgcgcgcgctcgaggacgcggacggcgtgcggatgactgccacagagaccgcggaccgcgtgttctcaaagatgttcagccaaaccatcatgctgctgttgggggggttcaccatggcagctgccatcaccaagcacctcatcgccaagcggatggcctttcaggtgctctgccgcgtggggcggcggccggggaatgtgttgctggcctctatgttcattgccctcttcagcagcatgtggatctccaacgtggcctcgcccgtcatgtgctacggcatcgtgcagcccatcctgcgcacgctggcgcccgacgaccgcttcgcgtctgcgctggtgatggcgatcgcgctggcggccaacgtaggcggcatgacctcccccatcgccagcccgcagaacatctttgcagtggagcgcatggcgatggatggcaacccgcccagctggctggcctggttctcggtctccatgcccgtctccatgttctgcatcctcgtgctgtggcgcatcatcctcgcggtgtacaggatcgggccgcacactgcggaggtgcgccccatgcggccgctggacgacgtcttcacgatgcagcacgtgttcatcattctcatcagcgtcctcacgatgggcctctggtgcgccaacacgtggctgctcagcgtgctcggcggcatgggcatcaccgcgctgctgccgatggtggcattcttcggctcgggcatcctcaacaagctcgacttcgagagcttcccctggagcgtggtgatgctcgccatgggcggcattgtgctgggggaggcggtcaagagcagcgggctgctgggcttcatcgcgcagggcatagtggggctggtggatgggttcacagtgtggcaggtgctgatgatcttcggcgcggtgatcggggtggtcaccagcttcatctcgcacactgtgggcgcgatggtcatcctgccggtggtgcagagcgtgggcgaggagatggccaaggcctcgggcgtggcgcactacaagctgctggtgatgggggcggcgctgtgctgctcgggtgggatggcgctgccggtgagcagcttccccaacatgacagccgcctcgctgacagaccccacc

SEQ ID NO: 140 - DNA, 1497 bp, unidentified
  source 1..1497: mol_type = other DNA; note = Hafniomonas reticulata
actgctggcaatgccgagaaggctgagggtgagctgagtgctcatctaagagagcaagtgcagtacgagcgcaatacagtgtggcgtgacatggtggcgaacgaacgcacgaagaccgccgaaagcagcaagcaagttcgaacgctatcgcaatctattgtctgcatcgccatcgtcgctgttgtcgctctgatccatgccaacatcttcccagatcaaccctccaaaaacaactgccttgcgatgctcgtgtttgtgagcatcctatgggcatccgaagcagttcctctgtttgcgacgtctatgatggtgcctttgcttacggtgcttcttggcatttgggtggacccttctcaaaaaccacctaagaggctggaccacacggctgctgcttctgctgttttctctgctatgttcaaccaggtcatcatgcttttgctcggtggctttgccatcgccgccgccctctcaaaacacttcatcgcgaagcgtgttgctgtcgccattctctcccgtgtgggtcgcaaacctcacaacatcatcctcgcgtctatgtgtgtggcggccttcatgtcgatgtggatatcgaacgtcgcggcgcctgtgctttgcttttcgttgatagcgcctatcttgcgaacgttggatgcatcgcacccctttgccaaggcgttggtgatgggcattgctttggcgtcgaatgtgggagggatgacgagccctattagcagcccccagaacctgttcgcgattgagcgcatgtccatgcccccttcctggttgtcttggtttgcggttgcgcttccagtggcaggtatttctatcattctctgttggctcatcattctcattgtataccggccgtggacaaaagtgaaggaagtccgacccttgaaacaagatgatcctatcacgtacacgcaggcattcgtcgttctcgtcagcgtcgtcacggttggtctttggtgtgcggagtctcagctagaaagtgtgtttggacagatgggggtgattgcaattttgcccatgctcgcattctttgcaactggtattctcaacaaggatgacttcaacggtttcttgtggaacgtggttatgcttgccatgggcgggttagtgctaggtggagctgtgaagaactgtggattgttagaagacattgcaaacgctatcaagaaccaagttgctgacttggaattgttccaggttcttgtcgtgttctgcctgctcgtgctcttctgcacgacattcatttcccatacagtgggcgccattgtcattctacccatcgttcagcaagtgggcgaaagtttccctggcacccctcattcaaagttgctggtcatgggcgccgccctcatgtgcagtggtgctatgggcctacccgtgtcaggcttcccaaacatgaatgcggtcgcactggaggacgccaccggacagacgtacgtgcagactcttgattttctcaaagtcagtgttccttgctcaatctgtgcg

SEQ ID NO: 141 - DNA, 702 bp, Tetraselmis sp.
  source 1..702: mol_type = other DNA; note = Tetraselmis chui
ctactgagcgcagccgtgctttggtgcacggaggccgtcccgctttacgtcacgtcgatggcgctcatcttcgctgtggtcgttctgcgggcgatgttggacggggacggcgtgcggatgtcagcgcaggacaccatgaagcgcgtattctctaagatcttcagccagactgtcatgcttctcctagggggcttcactatggcggcagccctgtcgaagcacctgatcgccaagcggttggccatcggggtgatgtcgcaggtgggccgtaagccatccaacgtgctcatcgcctccatggccatcgcactcttcagcagcatgtggatctccaacgtagcggcgcccgtactgtgttttagcatagtgcagcctatcctgcgtactctacctacggatgaccctctaggtgctgcgctggttatgggtatcgcgatgtcctccaacatcggcggcatgacctcccccatcgccagccctcaaaacatattcgccatcgaacgcatgtccctggacggccaccctcctagctggattacctggtttgccgtctccatgcccgtgtctatcacctgcatgctgctcgtatggagactcctcctcgccatttacccggtcagtagcaatcaggatgtccgtccgctgcggcaactacacgacccgttcacgtttcagcacgtgtacataatcatcgtgtgctta

SEQ ID NO: 142 - DNA, 1302 bp, Volvox globator
  source 1..1302: mol_type = other DNA
ctgtggtcgttggaggccattcccctgttcgtcaccagcatggctcttccgctcctcatcgtcgtgctgggggtgctacaagactcacccaacacagagaaacctgccactcgactcactccgcagcaggcggcgaccgccatattccatgccatgttctcacagactatcatgctgctgttgggcggtttcgctatcgctgcggcgctctccaaacatgccattgccaagcaggttgcggttgccattttgtccagagtgggtcgtaagcctcgcaacgtgctgctggccgccatgttcaccgccacgttcgcctccatgtggatatcgaacgtggcagcacccgtgctgtgtttcggattggtacaacccattttgcgcaccctggaccctggacaccaattcgccaagtctctagtcatgggtatcgccctcgcctccaatgtgggtggcatgacctcccccatctcgtcgccgcagaatatcttcgcgatagagcgcatgtccttggacggccaaccgcctagctggctgtcctggtttacggtggcgctacccgtgtcgattgcagccaacatgctctgctgggcgatcctgctggtggtgtaccaacccgaccgagccatcgcggaggttcggcccattaaacccaacaccgaccccaccaacggcacccaggtgtacatcatcgttgtggcgctgctcacggtgtcagcatggtgtgcaaacacattcctacagagctacacgggtgagatgggtatcatcgccatcttgccccttgtggcgttcttcgggttcgatgtgctcaacaaggatgacttcaactcctttctgtggaatgtggtcatgctggccatggggggactcgcgctgggtgaggccgttaagagcagtggcctgctggccgccctcgcctcggacatcagcgacctggtgttgggtctcagtctctggcaggttacactcatcttctgtggactggttctggtggccaccactttcatcagccacacggtgggcgccatggtcatcctacccatcgtacagtccgtgggggagcgaatgattggcacaccgcaccccaaactgctcgtcatggcgacagcactgacatgttcaggtgccatgggtctgcctgtgagtggcttccccaacatgaacgcggtgagtctggaggacgccacgggcaaccccatcgtcagcacaaaggactttctcatggtgggggtgccctcgtccattgcggcgtacggcatcattgtgacgctggggtacggcttgatgctgttggtgggcttt

SEQ ID NO: 143 - DNA, 2421 bp, Volvox aureus
  source 1..2421: mol_type = other DNA; strain = M1028
atgaagttcacacatcagctgaagtttaacagcgtcccggagtggcgcgagcactacatacagtatgcacatttaaaaaagtacatatatgctcttgcaaagagggaggcggatttgcaggctgggggccagcttcatgatgacgagttgctcaccccacttgttccggagacttcacgccagggctttagcgaagaaggctttcaacgggaacttgatgcccagcttgcttcaattctttcattttttgctgtcaaagaagcagagctgcttgctaaagtgtcggaactggaattagacgtgcaaagtttggaaaaaattccgaatcggcaagaagcatcctcgatgtctcgcattagcggtaatccatcgactaccggctatcacagcagcagcagtcctagggggcctgtcggtttgccgtcgatgtcgttgatgtccgtttctccctccacgcttgacctggcacggatggtggactctactccgccagaggacttccgcaaagtgcgagtcaagttctgggaaaatccgccacggcacgttttctctccaaaccttaacacgcgacggcaaaagcttttagggcggtttcaggacctgttcattggcctgcacgacttgcgggagtatttgcacatcaacaaggaaggatttcggaagactatcaagaagcacgataagctgactcgctctgtcgatcttcgcgtgcgctggtggccgaacgtggaagtgcacttggcgcccgtagcgaagcaacaagagctagagagggcaattgcggcgctcacggatcactatgcggtgctgtacatggggggtgaccttacaaaagccgacgagcaactgtcgcgaggcctgcgtgagcatatcacggtggagcgcaatactgtgtggcgcgatatggcggcaatggagcgcaaatacgcggcggtgtctgtcaaacaggcgacagctcccagagatggtgggcggcaagctcatgtccggtgggctaagctggccgcgtgctgcctggtcttcgcatcgctgttgctatgggggggacccgtggagaatgggcaggtcaaccagcccagaaacaactgcttggcgttgctggtgtttgcatctttgttgtggtcactggaggccgtgcctctgtacgtcacaagtatggctctaccttttctgattgtcgccatgggtgtccttgttgaccacccaagtgattccaaggatccacctagacgcctcacaccgcagcaagctgcaccagcgatcttccatgccatgttctcacagacaatcatgttacttcttggtggtttttcgatagcggccgctctctccaagcacgccatcgcaaagcaggtggctgtggccattttatctcgtgttggaaggaaaccacgcaacgtactgcttgctgcaatgtttacagccacattcgcatcaatgtggatttccaatgtggcggcaccagtactctgttttggcctaattcaacccatcctgagaaccctggacccgggacatccttttgcgaagtccctggttatgggcattgcgctggcttccaatgtcggaggaatgacatcgcccatatcttcaccccaaaacatttttgcaatagagcgcatgtcgatggatggacagccgcccagctggctctcttggttcgctgtagcaataccagtgtccatcacatgcaattttctctgttgggccttactccttctggtctaccaaccaggacgagctttgggtgaggtccgaccgctaaaacccaacacggaccccattaacggaacacaggtgtacatcattgttgtgtcgatgttgactgtggcagcctggtgtgcgaacacattcctccagaggtatactggcgagatgggcatcatcgccattgtccctttggtagcttttttcggtttcgacgtactcaacaaggatgatttcaactcgtttctatggaacgtcgtcatgctggccatgggcgggttgtctctaggtgaagcagttaagagcagcggtttgcttgcagcactcacaaacagtattagtgaactggtgacaggcttcactatgttccaagttaccctcatattctgtggcctggtgctgctggcgacaacgtttatcagccacacagtaggggctatggtcatcctaccaatcgtgcagagtgttggagagagcatggctgggacaccacaccctaagctgttggtcatggcatcggcgctcatgtgctctggtgctatgggcttgcctgtcagcggatttccgaacatgaacgctgtcagcttggaggatagcactggcaatgcaattgtcagtactaaggacttcctgtacgtcggtgtgccttcatcggtgatggcttatggcataatcgtcagtctagggtatgtgctcatgctgttggtaggtatg

SEQ ID NO: 144 - DNA, 1818 bp, unidentified
  source 1..1818: mol_type = other DNA; note = Ignatius tetrasporus
gacatgtactccaaactggaagatctggtcaactttctcgaactgaatcgtgaaggcttcaggaaagcattgaagaagcatgataaagtgactcaacgcaatctgtctccaatcttgctgccggaagctttggaggcactcaatgtgcaggacaacaagaacgcaattgaagaaaggaagcaggatgtcgtgcagtgttatgcaacaagtcagcagaaaggcgaagtgcttgccgcaaccaatgtactcaaacgccagcaaagggagatggtggagtttgagcgcagcacggtatggcgggagcacatggcagtggagcggcagcacgcgcaagcgaccgcgaaagccgtggagttgacaggctggcagcattggtggagcagccatcgcaatctgatttggatcgcggttgctgtcgccgcgttcttcattgtgctgcttgttcccatgcccatctttgacaccgttgaacagcacaactgcgctgctttgctaactatggcgggcattctatggtgcttcgaagcgctgccgctgttcgcaacgggcatgctggtaccgtttcttgttgttgtgctgcgcgtgatgcgcgttgccgacacaccacagcacgcttgcacgcatgactgccggctgtcggcgcctgatgctgcgagcgctgtgtttcacagcatgatggaccaggtgatatttttgctgttgggaggctttacgatagcagcagcgctgacgaaacacaacattgcgaagcaactcgcatcagcaggattggcaagtgtgcgcgacgcgcctggcaaactgttgttcgcagcaatgaacattgcgacagtgtcgagtcttttcatctccaatgttgcggctcctgtgctctgcttctcgctcgtgcagccgattttgcgcacgcacaagcacgatcatccgttttccaaggcgctggttatcggcatcgcgcttgcatcaaatattggcggcatgacctcccccatttcaagtccgcagaacatatttgcgattcagaagatggatgaagacggacgagcaccaagctggctcgtttggttcgcagttgcgctgcctgtcgcgtttgcatgcaacgccgcgtgcttcacagtcatctggcacttctacaaaccctacaaaacacgcacggctattcgtctcccgaaacttaccgacaaactgaactccactcaggtgtttgtgatcgtcgtgagcttgctgacggttggattgtggtgcgcgaacgcgcagctcgaaaagtactttggcaaaatgggaatcattgcgatcctgcctgttgtgttgttttatggaagtggcgttttaaacaaagacgacttgaacaactacttgtggaacgttgtcgtgctggcaatgggtgggctcgcgcttggagaggctgtgaagtcatcgggcctgttacacgtcatcgcgaatctgttggcggatgctgttggatcgttggacctgtggctcgtgctcatcgtgttttgtgggtttgtgctgatcggtaccactttcatctcccacacggtgggagcgatgatcatcttgcccatccttcaaacagttggcaagaatcttccaggcgcgccgcatccgcagctgttagtcatgggagccgcattgatgtgcagcggcgccatgggtttgtccgtcagtggctttccaaacatgactgcagctgctcaacaagccagcaccggcgagcattacatcaattcagctgatttcctgcaagttggcattccgtgttccgtcattacatatggcatcatcgttactcttgggtacggtttgatgctggctgtgggactg

SEQ ID NO: 145 - DNA, 2430 bp, Gonium pectorale
  source 1..2430: mol_type = other DNA
atgaagtttacccaccagctgaagttcaacagcgtcccggaatggcgtgaacactacatacagtatgcgcatcttaagaagtacatatacgcgcttgcgaagaaggaggccgatttgcaggccggagcgccgacaattgaagaaggtccgcttgcaccgctgttgcaggatgcgcgtgctacacagggcccctccgaggaaggctttcagcgcgagcttgacgctcagctggcagctctcttggccttctttgccgttaaagaggcggatttgcttgcgaaggtgtccgggctggaactggacgtgcagagcttggaaaagattcctggtcgtcgcgaagcttcaacattatcacgtctggggataacaggtggcccatgcagcagctcagatgccattgcccctggtgccgcggtgggcgcgactgtagcgcccttgaccacactgacaatggacgcgagcccgtccacccttgacctggcaaggatggtcacatgcacgccgcctcaagaccatcgcaaagtgcgcgtcaagtattgggaaaatccacctcggagcacattctcgcctaacctcaactcgcggaaaatgaagctgcagggacgcttccaggacctgtacattgggttgcacgatttgcggcagttccttattattaataaagagggctttcgcaagatcatcaagaagcacgacaagctaacccgcatggtggaccttcgtgattgttggtggccaaacgtagaggcgcacctggccccaaccaccaagcagcaggaacttgatcgagctatcgccgacatctccgaccactacgccgttgtttacaccggaggcgatgtggccaaggcggaggaacagctttccaggggcctccgtgaacacatcacttttgaacgaaatacagtctggcgtgacatggcggccatggagcgcaagtacgccgctgtgtcagtcaagcaagctgccccaccgggtggtgccaaggccagccggctgcgcgactaccttcagtggacaaagcttgcactgtcgtgcgcagtgtttgcaatcttgctgaacgtcgatgtgtggccagggccacaaaacggcccgcggaacaactgcctggcactactcgttttcgcctcacttctatggtcattagaggccgtcccgctcttcgtgacaagtatggccatcccttttctggttgtgacgttagaggttttgacagacgggacgaaggatccaccgcagcgcctgactcctcagcaagcggcatctgtcgttttccatgcaatgttctcacagaccgttatgctcctgctgggtggcttctctattgcggctgcgctgtcgaaacatgcaattgctaaacaggtggcagttacgatcttgtcccgagtcgggcgcagaccacgcaacgtccttcttgctgccatgttcaccgcgacatttgcctcaatgtggatatcgaatgtggcggcaccggtactatgctttggccttgtgcagccaatcttgaggacgttaaatccggggcatccatttgctaaggcattagtgatgggcatcgcactggcgtcgaatgttgggggtatgacatcaccaatatcatcgccacagaacatcttcgctatagagcggatgtccatggacggccacccccctagctggctttcatggtttgctgtcgcgctgccagtgtccatcataattaacctggtatgctgggcattactcctgttggtctaccagccagagcgctacatcactgaagtgcggcgcgtcaagccaaatacggacccagtaaacgggacacaggtgtacattgtgatagtgtcactgctgaccgttgtctgctggtgtggcaactcatatctgcaaagatacaccggcgaaatgggcattattgctattgtcccattggttgcgttctttggatttggcgttctcaataaggatgatttcaactcgtttttgtggaatgttgttatgctggccatgggtggcttgtcattaggtgaagccgtgaagagcagtggccttttggcagccctcgcgttagacatcagcaatctggttaccgggctcagcttatggcaggtcgcggccgtattctttggcatggttttggtggcgacgactttcatcagccatacggttggcgctatggtcattctaccgatagtgcagtctgtcggcgaggccatggccagcccgacgcacccgaagctgcttgtcatgggcgcggctctcatgtgctcaggcgccatgggtcttccagtaagtggctttccgaacatgaacgccgttagcctcgaggacagcactggcaatgctattgttagcacgaaagacttcctcctcgtgggtgtgccatcatctttcttcgcatatctggtcatagttacgcttgggtacgtcctgatgctcttggtagggtta

SEQ ID NO: 146 - DNA, 2403 bp, unidentified
  source 1..2403: mol_type = other DNA; note = Planophila terrestris
atgaagttttcccactccctcaagttcaacagcgtcgcagaatggaggcagcactacataaattacggcgccttgaagaagctgtcttatgcgatcgaaaagcaggaggaggaaggccggcagcgcgagatgtttgcgcagcaaagcatggacctgcggcagcgctcgatgcgccagagcgtcgatctgccgcggggcgcagtggaggaggacgaagcccggcggcgcgcgtcgctcgacgcatcctcctcgttcacgggctccgttcagcagccgctcctgcagcggctctcgtctggcctgggcgggtctttgcgtttgtccatgctggagcgcagtgcggaagagggcggcaaggtctcgcaggccgacttccagcgcagcctcgactcggagctgcataagatagtggacttctacatcacaaaggaggcggagctgaagaaggagcttgcggccgcggagctcgatgcccgcgccgccgaagcgtcgtctgcgggatcgtctggctttgcagagatgcaggcggagcggctgccgcggccgtcgttctggcggaccgccgcgtccgacgcgctcaaagccaagatgcacgagcgcctgtgctcgctgtacgtgcagctggtggacctgctgaacttcatcgagctcaaccgcacgggcttccgcaagatcctcaagaagcacgacaaggtgaccgacgtgtcgctcatgaccgactacatgcccgtcgtgaccagcaagctgagctccaagcgcgaggaggaccttggggcgatgatcaatgaagtcatcaagctgcacgccatggtaatgcacaactcagacacgaacgcttcggaggtcgacctcaagcgcaacttgcgggatcacgtgacatatgagcgcgacaccatctggcgcgacatggttgcgctggagcgccgcaacattactgtcaaggtgccggagggggcgccgacgggtgggtttgcaaaatggtggcagatataccacacgcccgtcatggtggcggcggcgctgctgctctttttcgtcatcctcaacatcgacatctggccgaacgacacgcccaagcgccgctgcgccgcgatgctcgctctcctcgtcacgctgtggaccagcgaggctttgccactgtacgtcacgtccatgctggtgcccctgctgactgtgttgctgcgcatcctcccggacgaagctgccccagacggacaccctcagcagctgccggcgccgaaagccgcggaagccgtcttcaaggtgatgttttcgcaagtgatcatgctgctgcttggtggctttgccatcgcaggcgctctctcaaagcactacatcgccaaggccatggcgtccaatatcttgtcccgcgtcggcactcgcccacgggacgtcatcctggccaacatgtttgttgcaacgtttgccagcatgtggatctccaacgtggcggcgccggttctgtgcttctcgctcatacagccgatcctgcgcacgctgccgagtcaccatcccgtcagcaagtgcctggtgatgggcatcgcgctggcgtccaatgtgggcggcatgacgtcgccgatctcgagcccccagaacattttcgcaattgagctgatggcgcgcgacggcgcagcgcccaactggctcacgtggttcgtggtcagcatcccggtgtcgatcgcgtccaacctcttcatctgggccatcctgctggcggtgtaccggcccgggctcgcgatccgagaggtccgccacatgcgccgcgtgcaggagccgatcaccaaagtacaggtgtatgtggtcgctgtcagcagcctgacggtcgcgctctggtgcgccagctcagcgctggagcagtacctcggcagccagggcatcatcgcgatctttccgcttgtcatgttcttcggcctcggcgttctcgacaaagacgacttcaacaactttctctggcatgttgtcatgctcgctcaggggggcctcgtgcttgggctggcggtgaaggggtcgggcctgctggcggacgtggcaggcgcgatccgcggcgtcacggccggcatgtcgctcttcggcatcctcttcacgttctgcgcgctggtgctcgtatgcacgaccttcatctcgcacaccgtcggcgcgatggtcatcctgcccatcgtgcgctccgtggggcagcacctctccccgacgccgcacccgcggctgctcgtgatgggcgccgcgctcgcgtgctccggcgcgatgggcctgcccgtgtccggatttccaaacatgaacgcggtttcgcttgaagatgccactggaaagacttacgtgaacacactcgattttctcatggtgggcctgctgggaagcttgactacgttctgtgtcattgtgacgctgggctacgctctgatgatg

SEQ ID NO: 147 - DNA, 2469 bp, Pteromonas angulosa
  source 1..2469: mol_type = other DNA
atgaagttcacgcaccagctcaagttcaactccgtgccggagtggcgcgacaactacattgactatgcgcacctcaagaagttcatctttgccatcgcgcgggcggagcaggatgacatccagcagctgcatggcggtgctgatggcacatccatgcccttgctacagcacacggtcaccatggggcacgacaaggtggacgccacggaggacaacctccgccaggcgctggacaaggagctgcagcgcgtgatcagcttctacatgaccaaggaggcggagacgctggccaaggtgacttccatggagcttgagatcaacacgctggagatgacccgtgccccccgaggcacctccatggaccacatgcagggagcccagcgtggaggatctggtggatccggtggctccggaggcggagtggacctgaaccagcagctgccatcgcctccccagggccttgccacggacgtggaggccacccccccagcggcgcacgtgagcgccagcatggcagcccccaagtcacctggcaccatgtcgcgccagatgcgtgtggacttctgggcgcgcgccaaccccggcgccaggcacggcgggtcgttcggcggcggctccgcggccttcctcttcgtgcgcgagctgcagtcccacaaggagcgcctgcgtgtcgtgttcagcgacctctacctgcagctgcacgacctgctcaacttcctgagggtgaacaaggagggcttccgcaagatcatcaagaagcacgacaagatgacgagcagcaacctgaaggagcactactggccagtcctggagtccaagtaccccatcgtgcgcgccgacatgctggaggcaaccatcaactcgctggtcgacctctacgcagtcatctacaaccagggcagcgtggagctggccaaggaccacctggacaagctgctgcgcgaccagatcaaggtggagcgcaacacggtgtggcgcgacatggtggcccaggagcgccgcaccaccgcggccgtggtggagggtgcggtcaggcggccctggtgggcccagctgaccccccacatcgcactgctgagcagcgtgctggtgtttgcagtcctgctgtccatggaggacatcttcgagggcgagccggagaagcagaactgcctggcgctcctcatctttgtctccatgctctgggccaccgaggcggtcccactctacgtcacctccctcgccatccccctgctggtggtggtgctcaaggtgctgatggacaagagcgtggacccccccgtgcgcatgacggcgcagcaggccgcgcccgccatcttccactccatgatgtcgcaggtcatcctgctgctgctgggcggctctgccatcgcctccgccctcaccaagcacttcatcgccaagaagctggcccaggtggtgctctcgcgcgccggccggcaaccacacaatgtgctgctggcgctgatgttggtggccgttgttgcctccatgttcatctccaacgtggctgcgcctgtgctgtgctggtcgcttgtggacccgatcctcaaggcctttgacgcagagaacccgttctccaagtcgctggtcatgggcatcgcgctggcctccaacatcggcggcatgacctcccccatctcctccccccagaacatctttgccatcgagcgcatgagcatggacggccacccgccttcctggctggcgtggttcgccgtcgccctgcccgtctccttcatctgcatcctggtgtgctggggcctgatcctcgccgtgtatcgcccctggaccaaggtggctgaggtgaggcccctgaagccaagctccgacaaggtcaccttcacgcagttctacgttgtggccgtcaccgccgtcaccgtcaccctgtggtgcttcaacacccagctgcagccgtacaccggggagatgggcgtggtggccaccatccccatcatcgccttctttggctttggtgtgctcaacaaggacgacttcctgtcctccccctggctggtcatgacgctggccatgggcggcctgggcctgggtgaggcggtcaagagcagcggcctgctgctgtccatcgcacacaccatcggcgacgtggtgcagggcatggacgtcttcaccgtctgctgcatcttctgcgccctggtcctggtctgcacctccttcatcagccacacggtgggcgccatgatcatcctgcccatcgtgcagtcggtgggcgagcagatgcccggcccccaccacgccaagttgctggtcatgtcctccgccctcatgtgctccggagccatgggcctccctgtcagcggcttccccaacatgttcctcatcagcaaggacgacggcacgggcaagaactacatcaacacgctcgacttcatcaaggtgggcgtgcccggctccatcggcgcctactttgtgattgtctccgtcggctacctcctcatgctggcggtc

SEQ ID NO: 148 - DNA, 2340 bp, Asteromonas gracilis
  source 1..2340: mol_type = other DNA
atgaaattttcgcaccaactgaaattcaatagcgtagctgactggaaagagcattatattcattatgctaaccttaagaaaatcatttacgaaatcgctcgtctggagcaggcgcgagccaatccagatgccggagaggtcacagaattgggggagcccttactttccagaccaccagttcaaaattacgaacttgctatttctacaaaggaatctgaatttgtcggggagcttgatcgtgagctggctcatatcattacattcatcttgcggaaggaggctgagttggtgagccagcttgaagcactagatctcgaggtccacagcttggagagtgcagatccccaatatcgtaagagcttggatagggattttttggaccaggatgctgcagtaggagctgagaatgggaccgggtatcaagcgggcattccagctcgacctgagcgaatcaagttctggtcccaaggtgctgaacctcaccttgctgctcgtgatgcgcgaaatgtggcccaactgaaacctgcacaaagagaggctctgtcacagaaattcgtcgacctttttaccactttaaatgacctattggaatacctggtcctcaaccgcgaaggcttcaggaagctgatcaagaagcatgataagatgacgtcttcagcaagcatgaaggaaagctactggcctctgattgagcagcgttatccagagcataagagggtttcaatgggacagaatattgagcggctggtggacctgtatgctattctgttcgaggggggtgacacgtcgtcagccagagaggccctgtcacagaatctgcggcagcatatcaaagtagagcgaaacactgtctggagggacatggtggcaatggagaggcgcacagttgctgcaacagtagacgcgcctaagaggaagagagcctggttcagcactcacaggaagcacctgtccctcctgctggcttcgattgtgttcgcatctatgctcagcttgaagctttttaaagagcctgagaaatcgaattgtgcggcgattctggtgttcgtgtcgcttctctgggcgagtgaagctatccccttattcgtgaccgcaatggtggtccctgtcctggtggtgtcgctgcgtgttcttgttgacgactctagcgcaaagcatcccatccgcctttcttccacggatgctgccaacgccatcttccacgccatgttctcccaggttaccatgctgctcctgggtggcttcaccatcgcagccgccttgtccaagcacttcattgccaaacaaatggcggttgcagtgctgtccagggtgggcaggcttccgcgcaacgtccttttggcgtcgatgtgtgtcgctgccttcgcatccatgtggatctccaacgttgccgcaccggtgctgtgcttctctctagtgcagcccattctgcgcacactggacgtgagctcacccttcgcgaaaagcctcgtcatgggcattgccctcgcctcaaacatagggggtatgaccagccctatctcctccccccaaaatatcttcgccatcgagcgcatgtccatggatggcgtcgctcccagctggctgtcctggttcgcggtggcgctacccgtctctttcatcagcattatcctctgctggctcctcctcctcctcgtgtatcggcctgggatgtctacaaccgaggtccggcctctgaagccgtatacggaccctatgaacatgacgcaggtgtacgtgattgtggtgtccatggtcacggttttgctgtggtgcgccaactccgaggtgcagcagtttgtggggaacatgggggttgtggcggtgctgcccatgattgctttcttcggctttggggtgttgagcaaggacgacttcaatggcttcctatggaacgtggtcatgctggccatggggggctcggccctgggggaggctgtgaagagcagcggcctgctgtccacgtttgcaaatgacatcagcgggcaggtacatgggcttgacttgtggaccgtcagtgcgattttctgcggtgtcgtgctcatctgcgccacgttcatcagccacacggtggcggcaatggtcattctccccattgtccagtccgtcggcgaggcgatgcaggagaaccctcatccaaagctgctggtcatggccactgccctcacgtgctcagctgccatgggcttgcccgtgagtggcttccccaatatgaatgctgtgtcactggaggatggcaccggccaaaccttcgtcaacaccctggactttttgaaggtgggcgtccccagctccgtcgctacgtatttcgtgattatttctgcgggctactacttcatg

SEQ ID NO: 149 - DNA, 1887 bp, Haematococcus pluvialis
  source 1..1887: mol_type = other DNA; note = Haematococcus pluvialis-B
cgggtgcggttctgggcagagctgggcatgcggcgcggcggcagagacttgcggtttgcccgggatgtcatgcgcatccgcttccatgacttgtacaccagcctgaacgacctcatagagtacctcagcttgaatagggaggggttccgcaagctcatcaagaagcatgacaagctgaccagcacatgcctcaaggaggcctactggcctgactttgagcgtcgctaccccatgaagcgcaaggaagagctggagcggcaccttgatcgcctcatagagctgtacgctgtcatgtttgctggcggagacacgcgcaaggccagggacctgctgctgaagacgctgcgagagcacatcaaggtggagcgcaacacagtgtggagggacatggtggcactggagcggcgcactgtggcagccactgtgggcgctgcttctggggtagctcgcctcagcaagtacaaggcatacagcgagcgccttggcctgctggcagccctcctggtctttgctgcgctgttgtgggcaccagtgtttgaggagaaggagaagagcaactgcctggccattctcgtcctggcatccatgctttgggccacagaggccattcctttgtttgccacggccatgctcatccccgtactggtggtcatgctcagggtgttggtggaccacgggcggcctgcaggtgcccagcgcctcacccctcaggaggcggcccccctcatcttccacgccatgttctcccaggtcatcatgctgctgctcggggggttcaccatagctgctgccctttcaaaacacttcatcgccaagcagatggcagtagcagtactgtccagggtggggcgtaagccacacaacgtgctgctggcctccatgtttgtggccatctttgcctccatgtggatctctaacgttgctgcgcctgtgctgtgcttctccctggtgcagcccatactcagaaccttggatgtaaacactccctttgccaaatccctggtcatggggatagcccttgcctcaaacatcggtggtatgacctctcctatctcctcaccccagaacatctttgccattgagcgcatgtccatggatggcaacccacccagctggctcagctggttcttcgtggcactgcctgtggcaatcatcagcaacttcatctgttgggccgccattctcctggtgtaccagccctggcacaagacgtctgaggtccggcccatcaagcccagctcggaccctgtcacctggacgcaggtgtatgtcatatttgtgtcgctggcgacggtgggtttgtggtgcggcaacgtggccctgcagaagtacacaggggagatgggtgtggtggcggtgctgcccatggtggcattctttgggttcggtgtgctcaacaaggatgacttcaacggcttcttgtggaacgtggtgatgctggccatgggcggttcagccctgggggaagctgtgaagagcagtggcctgctgttgaccattgctcaaggcatccaagagatggtggatggcctgaacctgtggactgtcaccatcatcttctgcgcctgtgtgctggtgtgcaccaccttcatctctcacacagtgggcgccatggtcatcctgcctatcgtgcaaagcgtgggggagagcatgcccggccagccccaccccaagctgctggtcatgagcgcagcactcatgtgctcaggcgccatgggcttgcccgtcagcggcttccccaacatgaatgccgtgtccctggaagactccacagggcagacatatgtcggagcactcgacttcatcaaggtcggggtacctagctcgatcctagcgtacgccgtgatcatcacggtcgggtattcgttgatgttgatcattggcttc

SEQ ID NO: 150 - DNA, 1140 bp, Chlamydomonas sp.
  source 1..1140: mol_type = other DNA; note = Chlamydomonas bilatus-B
cagaccatcatgctcctgctcggtggctttgccatcgccggcgcgctgagcaagcacttcatcgccaagcagctcgcgatcgccgtgctgtcgcgcgtcgggcgcaagccccacaacgtgctgctcgccgccatgtttgtggcgacgtttgcgtcaatgtggatcagcaacgtggcagcaccggtgctctgcttctccatcatcatgcccatcctcaagacgttggacacagccagctcgttcgccaagtccatggtgatgggcatcgcgctcgcgtccaacgtgggcggcatgacatcgcccatcagcagcccgcagaacatctttgccatcgagcgcatgtccatggacgggcagcccccctcctggctcgcctggtttgcggtcgcactgcccgtcgcaacgctctgcaacctgctgtgctggctgctcatcctggccgtgtaccagccctggcgcaccatcaatgacgtgaggccgctcaagcccaacacagaccccatgaacttcacacaggcttacgtcatcttcatctcattggcgacagtggggctgtggtgcgccaacaccagcttgcaacagtacactggcgagatgggcgttgtggctgtactgcccctggttgccttctttggctccggagtgctcagcaaggatgacttcaacggcttcctctggaacgtggtcatgctggccatgggaggcctggcgcttggcgaggctgtaaagagcagcggcctgctccagtccatggcagagggcatcacggaggtgacggatgggatggacctctaccaggtgctgctggtcttctgcctcatggtgctcatcagcaccaccttcatcagccacacggtcggcgcgatggtgatcctgcccattgtgcagagcgtgggtgaggctatgccgggcagcccgcaccccaagctcctggtcatggccagtgctctcatgtgctcaggcgccatgggcctgccagtcagcggcttccccaacatgaacgccgtttcactcgaggactcaaccgggcagaactacgtggacacgctggacttcctcaaggttggagtgccaggctcggtactcgcctatggcgtcattgtcaccctgggctacaacctcatgctcatggtgcgcttc

SEQ ID NO: 151 - DNA, 1932 bp, unidentified
  source 1..1932: mol_type = other DNA; note = Vitreochlamys sp.
atgaagtttagctctcttttaaagttcaactgtgtacctgagtggcgtgaccattacgttcagtatggccacctgaaaaagtacatatacgcgttggcgaaatgggaagcggaccacctacacgaaactcagcctccggacctggagtcgctcacgtctcccttgctgccaaccagtggactggggtctgcttatggacccagcgaggaggccttccagcgtgagctggaccagtcactgctggaggtgatccgcttcttcagcatgaaggaggcagagttggtgtccaagtgccaggcgctgctgctggagctggtctcggtggagaagctgccgtccgggtcgtccgcggggcgccgctccttcagcggcgcctccacgccctcgggtgccgccacgcccacgtcctcggcgccccacggctccaccgccaacgtcctggccggcgccaagtcgcgtctcaccgcctcgccgcagacgtcgccccacgtgacgctcagcggcgccaagggcgcgggggggctgggcggcatgcacttgtcgccctccgtcgtgcatctcatggacgtggctaaccacaaggaccaccgtacagtgcgtgtggaattctggcgcaagcccccgcgccgcctgttccagaacctggaggccgctcgcagcaagctcaagcccaggctccaggagctgtacattgctctgcacgaccttgccgagttcctccacctcaaccgcgagggcctgcgcaaggtcgtgaagaaacacgacaagctgacccgccgggtcacgctcaagaccaagtggtggccccaagtggagcacctcatcccgcccaccaagaaggaggaggtggacagggcagtttcggagctggtggacaactacgcggtcttgttcacgggcggctcaatggcggctgctgagcaggcgctcagccaggggctgcgtgactacgtgaccatggagcgcaacaccgtgtggcgtgacatggctgccatggagcgccgctttgcatccctcgcagtcaagaagggctccgcaagcttcattgccacctggtggacgcagccgctcaagatcgccgtgagtctgttggtgctgtcggtgctgctcaacgtgacgatctggcccgaggacgagaagaacaactgtctggcggtgctgctgtttgcgtccatgatgtggtcgctggaggctatcccgctgtttgtcacgtccatgaccatccccttcctcgtcgtctgcttccagctgctggtggaccattcgcaagacccacccgtgcgtatgaccgcgcagcaggctgcacccgccatcttccacgccatgttcagccaggtcatcatgctgctgctggggggctttgccatcgcagcggcgctttccaagcatgccatcgccaagcagatctcagtggctgtcctctcacgcgtcgggcgtaagccgcgcaacgtgctcctcgctagcatgtttgtggctacatttgcgtccatgtggatttcaaacgtggctgcgcccgtgctgtgctttggcctcatccagcccatcctccgcaccctcgacccaggccaccctttcgccaagtgcttggtgatgggcatcgcgctggccagcaacgtgggcggcatgacgtcgcccatcagctccccgcagaacatctttgccattgagcgcatgtccgtggacggcaaggcgccttcgtggctgtcgtggtttgccgtcgcgctccctgtgtccattgtgtccaacctgatctgctgggcggtgctgctgctggtgtaccgcccctggaccaaaatccaggaggtacgacccatcaagcccatcaccgaccccatcaacggcacgcaggtgtacatcatcgtggtgtcggtggtgacggtggcgctgtggtgcagcaacaccatcctacaaccgtacacgggcgagatgggc

SEQ ID NO: 152 - DNA, 2412 bp, Botryococcus sp.
  source 1..2412: mol_type = other DNA; note = Botryococcus terribilis
atgaagttttcccagcagatcatattcaattccgttcctgaatggaaagataactacatcagctatgcccaactcaaaaggttgatatactccgaggaagctgcacgtttagcggctggcagagatggagcccggggggcatcgatgcggctgcagcgtctgcggaagacagcgatgcagttcaaggacgatctcaagaaggaagctgacaaggtcgtcaggttcttccatgaggaggtggagagtatctggagcaggttccatctggtactgcacgagatcgaatgctttgagcagcaggagtggcttcccccctctgctgcgggcctggacacctccccgaccagcccccttctctccgccacatccatgcccgccacccccaaaacgccccccatgccaagcccgcgctccagccccttccagagggccggctcagccggcatggggctcttgcggacggtgacggggattctcccgcggccgaagcgcagccctagaactttgtctgggcccttgcttgaggtggaggatgggaccccccgggatgattctaagacctggatttggcagcaggcagaacccagtattgcccggaaacgggacgagctcaggggtcagttgtcggaggtgtaccaggatgcaaacaacatgatcgagttccggagattgaacctggacggctttcgcaagatcctgaagaagtacgacaaagtcctggaaggcctccccggagcagagaaactgtctgaatcacagtttcctgggatcaaggagagactcgaggcactggacttgacacggatgcaggaagtggaaggggaggtggtgaggttgtacgcgcgcgtgtgctgctccggtgtatatgcagttgctgaggaactgctcaagaagcagaagaaggatcgcatcgtctttgaccggagcaccgtctggaaggagatggtcgagcgggaaagaaaacgcagcgctgcgcacgtggagggcggggccgcgcctcgtgcctggtaccagcgccactggcagctcatggcctgcgccttctgcggggccgtgttcctcgcactgctctgggtgcccatctttgaggaggtggagaagcaacactgcgccgccctcttggccttcgtctcactcctctggtgcacggaggccctcccgctgtttgccactgccatgctggtgcccttcctggtggtgaccctcggggtgttggtggaccgcagcgttgaccctccccaccgcctcacgccccaggaggctgcccccgccgtcttcaagaccatgatgtcccaggtgatcatgctgctgctcgcgagctttgccattgccggagcgctgagcaagcacttcatcgccaagtggctggcgtcggtcttcctgtcgcgcttcggcaagcggccctctcgggtgcttctggccaacatgggggtggccacgtttgccagcatgtggattagtaatgtggctgcgcctgtgctctgcttctccctgctgcagcctatcctccgcaacctctctgcaaaggacagctttgcaaaggcactcgtgctaggcattgccctagcaagcaatctggggggcatgacgagtccgattgccagcccccagaacctgtttgcaatccagcagatgtcggtcgggggcaatgccccctcctggctccagtggtggttggttgctctgcctgttgccattataggcaacctggtgtgctggggcctgctgctgtggagataccagccgcccccagatgacgtgcgcgagctgcatgaagccaaggggttccacatcaacccgacccagatgtatgtggtggctgtctcactcctgaccgtggggctctggtgctgcaacggctacttgactccctactttggggagatgggcgtcatcgccatcatcccgctggttgcgttcttcggcacgggtgtcctggacaaggatgacttcaacgcattcctgtggaacgttgtgattctggcaatgggcgggatggcgctgggatctgcagtggacagctcaggtcttctgctcacaatcgcaaaaaagctggaaggtctggtgtcctcgcacggcccctgggtggtactggccattttctgcgcgctggtactgtttgcgacgacgtttgtctcccacacggtcggggccatcgtcatcctcccgatcgtgcgagccgtcggggagaccatgacggacccacacccaaagatgctggtcatgggggcagccctcatgtgctccggtgccatgggcctgcccgtctccggcttccccaacatgaacgcaatctcccttgaggacaagacaggcgtcaactacctgacgaccaaagacttcctgctggtgggggtcccgtcgtccgtcgccacgtggggcatcattgtcagtgtgggctacgtgctcatg

SEQ ID NO: 153 - DNA, 2412 bp, Eudorina elegans
  source 1..2412: mol_type = other DNA
atgaagtttacacataccctaaagtttaacgcagccgactcgtggagggaacactacattcaatatgcacacctgaagaagtacatttacgcccttgcaaaaagggaggcggacctccaggctggtggccatgtgcccgacgacgagtctctgcacgcgccgcttgtaccggagacctctcgctctggacagggcgtcagcgaagaaggctttcagcgggaactcgacgcccagctggccgccatcctttcattttttgctgtcaaagaagcggaactgcttgcgaaggtgtcggagttggagttggacttgcaaagtttggagaagatacctaatcggcaagaagcgtcgaccatgtcgcgcctcggtggcggcggtggggcagcgggcagcaacccaaccggcagccctggaactgccgccgtggcggctgtctctgcggtgttgccctccttgtcgattctctctgtcagtccctccaccctcgacctggcgcgcatggtgaactccactccgccggaggagcaccgcaaggtccgggtgaagttctgggagaacccgccaaggcacgtgttcttaccgagcctgcatgcacgacggacaaagctccagggccgctttcaggatctgtacatcggcttgcatgacctgcgggagtacttgcacatcaacaaggaaggctttcgcaaaattattaagaagcatgacaagttgacgcgtgcagtggatcttcgcgtgcgctggtggcccaacgttgaggcgcacctggcacctgatgccaagcagcaggagctggatcgggccattgcagcgctgacggatcactatgcggtgctgtacatggggggcgacgtggctaaggctgatgagcagctgtcccacggcctccgtgagcatattacggtggagcggaacacggtgtggcgcgatatggcggccatggagcgcaagtacgcagcggtgtccgtcaagcaggcagcggctcctgggggcctcagaggcagctaccgcaagctcgcggcctgctgtgcagtgttcgcagtgatgttgcacgtgaaggtttggggggaggatgaggacgagcccaagaacaactgcttggcgttgctggcgtttgcgtctctgctgtggtcgcttgaggccgtcccgctgttcgtaaccagcatggcccttccgctgctcattgtggtaacgggcgtactggtcgggcccgacaagcagcccctcaccccgcagcaagcggccccggccatcttccatgctatgttttcccagacgattatgctgctgctgggcggcttcgcaattgcggctgcactgtccaagcacgccattgcgaagcaggtggcggttgctattttgtcccgcgtggggcgcaagccccgcaacgtgcttcttgctgcgatgttcaccgcaacctttgcgtcgatgtggatttccaatgtcgcggcaccagtgctgtgcttcgggctcatccagccaatcttgcgcacccttgacccgggtcatccatttgccaagtctctggtgatggggattgcgttggcgtcgaacgttggaggcatgacatcgcccatatcatcgccgcagaacattttcgcgatcgagtgcatgtcattcgacggccatccccccagctggctttcctggtttgccgttgccctgccggtgtccataacatgtaactttgcctgctgggctgtgctcctactcgtttaccagcctgggcgggcaatcgcggaggtgcgacctatcaagccaaacacggatcccatcaacgggactcaggtttacatcatcgtcgtgtcgctgctgaccgtggctgcctggtgcgcaaacactttccttcaaaggtacactggtgagatgggtgtcattgcgattttgccgctcgttgcgttctttggcttcgatgtactcaacaaggacgacttcaactcattcctgtggaacgtggtcatgctggccatgggagggttgtcgctaggtgaggcggtcaagagcagcggcttgctggcagcgctcgcatcggacatcagcggggtggtgaaggatctcaccctgttccaggtggcggttatattctgcggaatggtgttggtggcgacaacattcatcagtcacacagtgggggctatggtcatcctgcccatcgtacaaactgtcggaaaggccatggaggggacgccgcatccaaagcttcttgttatggcagcggcgctgatgtgctcaggtgccatgggtttgccagtgagtggcttccctaatatgaacgcggttagcctggaggacagcactggcaatgccatcgtcagtacgcaggacttcttgtatgtcggtgtgccttcgtcaataatcgcctacggtattatagttaccctaggatatgtgctgatgctgctggttgggctt

SEQ ID NO: 154 - DNA, 2463 bp, Pandorina morum
  source 1..2463: mol_type = other DNA
atgaagtttacacaccagctgaagttcaacagtgtccccgaatggcgggagcactacattcagtacgcacatcttaagaaatatatttatgcattggccaaacgggaggcagatctgcaggctggaggcgacgaagatggactcttatcacccttggtaccggagacgtctcgggccggtcagggtgtcagcgaggaaggctttcagcgggagcttgatgctcagcttgcgtccattctttcgttctttgccgtcaaagaagcggagctacttgcgaaagtgtcggagctggaattggatgtacaaagcctggaaaagataccgagccgtcaggaggcgtctgtctccctatcacgcttgggcgctggcgggggatcagggggagggaaccccacgagcagccccgggtcggcggccgtgtcggccgtgtccgccgtgctgccctcgctgtcgctgctatcggtcagcccctccacactcgacctcgcacggatggtcagctccaccccgcccgaggagcaccggaaggtgcgggtcaagttctgggagaacccgccgcggcacgtcttctcaccaaacctacacgcgcggcgggcgaagctgcagggtcgtttccaggacctgtacatcggcttgcacgacctgcgcgagtacctgcacatcaacaaggagggtttccgcaagatcatcaagaagcacgacaagttgacgcgcgcggtagacctccgagcacgatggtggcccaacgtggaggcgcacctcgcgcccgacgcgaagcagcaggagctcgaccgcgccatcgcggcgctgacggaccactacgcggtgctgtacacgggtggcgacgttgagaaggctgaggagcagctgtcgcgcggcctgcgggagcacatcacggtggagcgcaacacagtgtggcgcgatatggcggccatggagcgcaagtacgcggccgtgtcggtgaagcaggcggcggcgcccggcttactgcggttcagcgccaatcgggcgcatgtgcgatgggcaaagctggcgctctgctgcgtggtgttcgccatcctgctcaacgtggacttttacaaggagaacgatatggagccgcccgacgtacaacgggcaaagaataagtgcctcgccttgctggtgttcgcatccatgctgtggtctctggaggcggtgccgctattcgtgactagcatggctctgccgtttctgatcgtcatgctgggggtccttatggactccgacggcaaggagcggctccagcccaagagtgcggcgcccgccattttccacgcgatgttctcccagacggtcatgcttctgctcggcggcttcgccattgcggctgctctgtccaagcatgcaattgcaaagcaggtggcggcggccatcttgtcgcgtgtgggacggaagccccgcaacgtgctgctcgccgccatgttcaccgcgacgttcgcgtcaatgtggatctccaacgtcgctgcaccggtcttgtgcttcgggctcatccagccaatcttgcggacactcgaccctggccacccctttgccaagtcactcgtgatggggatcgcactggcatcgaacgtcggcggcatgacatctccaatctcgtcgccacagaacattttcgccatcgaggagatgtccaagggtgccaatccgccgagctggctttcctggttcgccgtggcgttgccggtttccatcgcatgcaatctgatttgctgggccgtgctgctcctggcgtaccggcctggccacgtcatctccgaggtgcggcccatcaaacccaacacggaccccatcaatggcacacaggtgtacatcatcgtggtgtcgctcttgaccgtggctgcctggtgttcaaatacatttctacaaaggtacatcggtgaaatgggcgtcatcgccatcgtgccactggtggcgttcttcgggttcgacgtgctcaacaaagacgacttcaattcgttcctgtggaatgtcgtcatgctcgccatgggaggcatgtcgctcggcgaggctgtcaagagcagcgggctgctgtcggcgcttgcgacggacatcagcaacttggtacttcacttatccatcttccaaataactgtcattttctgcggcatggtgctggtggcgaccaccttcatcagccacaccgtcggagccatggtcatactgccgattgtttggtccgtgggcgaaaagatcaagggtgacgacccagccagtcagagccattcaaagctcctggtgatggctgcggcgctcatgtgttcaggtgccatgggtttgcccgtaagcggctttccaaacatgaacgctgtgagcctcgaagacagcacaggcaatccgatcgtaaacacgcaagacttcatctacgttggtgtgccttcgtcaatttttgcatacggggtcatcgtcaccttgggttacgtgctgatgtcgttggtgggcttt

SEQ ID NO: 155 - DNA, 2349 bp, Oedogonium foveolatum
  source 1..2349: mol_type = other DNA
gtgaagttcacacactatcttaagttcaacagtgttccagaatggagaggccaatacctggcgtatggcttattgaagaagcttatatacaagcaggagaagctgcttgctttaagccgggcggctccccatccagaatccgttgacattgaacatgaggagcccacggtggaaacgccattcttgcaagtcccgtcgacaccaccatctcagctggatttgtccccccgtcgctcctttgaccgcagcttcctctcgggtaaactgtccccccgcagcgcgagcacaacgggcaaccctgagattgagtttgtgaggttattgggctcggagcgcactcgcctcaatgagttcatcgcgagcaaatatacggagctgactgggcaactatccaacgtgacagaggtgatgcgagtgaaggagatggagggcggcctgccccactcggaccccaacccatacagcctggcggcacatcgggtggcgttctggagccaggctcccatgcagaaggcgcgcgagcagctcatcccgcagctggtggagctgtgcgtgttcctgacggggctcaaggactacgtggagatgaacaaggaggggttccggaagatcctgaagaagtgggacaaggtcaacgaggcgcggctgagcgaacaggaaatgccgctggtggagcagacgctggacgtggggcggaggctgcaggaccttgatgaagccattggtcacgtgatgtctttgtacgcgctgctcacctccaagggcaacatggacctggcctggcgcagcatgaaggagcaccagtcggagcacatcaggttccagcgcagcaccgtgtggcacgacctgatcgccctcgagcgccgcaccctgaccgccaccgccgtgcggcctgtagatgaggtgatgggctggtgggctgtcaaccggaagcacttcatgattgtggcctcgctgatggtgttcctgctgctgctggaggccaagacgtttgagggtgacgaagccgccccgcagcggaactgcctcgcgctgctggtctttgtctcgtgcctgtgggctacagaggcgatcccgctgtttgtgacgagcatgctggtgccgctgctggcggtgtcgctgcgtgtggtggtggtggacgggaagcggctggagccaccagatgcggccaccttcatgttcgggaaaatgttctcacaggtgatcatgctgctgttgggcggctttgccattgcagcggccctgagcaagcacaacattgcgcgcaagatggccatatcggtgctgtcccgcgtggggagagccccggggcgggtcttgttggcgaccatgatggtggccacattcctgtcgatgtggatctccaacgtggccgcgccagtgctgtgcttctctctcgtgcagcccatactgcggactctcgacacaaaccaccagtttgccaaggcgcttgtgatgggcatcgcgctggcctccaacgtgggcggcatgaccagccccatcagcagcccgcagaacatcttcgccatccaggtcatgtcaggcggcggccacagcccccccagctggacgcagtggttcgtggtggcgctgcccgtgtctgccgtgtgcaacgtgctcatctgggggctgctgctggcggtgtaccagccgcacaagcacatcaaagaggtgcggcccatccgcgccctccaggacgccttcacgctgcagcaggtggtggtggtgctggtcagcctgctcaccgtgacgctgtggtgcctcaatggcatgctggagccctacctggggtccatgggggtcatcgccatcctgccgctggtggccttcttcgggttcggcattctgaccaaggacgacttcaacgccttcctgtggaacgtggtcatgctggccatgggggggctggcggtcggggagtgcgtgaagagctcgcacctactgcagtccatcgcgcgcggcatccaggacacgactgccggctggtccctgtactgcgtgctggccatgttctgcgcgctggtgctgtgctgcacgaccttcatctcacacacggtgggcgcatttgtgatcctccctgtgctgcagagcgtgggagacgagatggcggcggccggacagccgaaccactccaagctgctggtcatggcggcagctctgatgtgctccggcgccatgggcctgccggtcagcggcttccccaacatgaacgccgtggcgctggaggaccaggcggggttcaattacgtggccacaatcgacttcatcaaagtggggctgctcagctcagggtttgcgtacgtggtcattatctcgctcggatatttactgatgctgatggtgggcttt

SEQ ID NO: 156 - DNA, 564 bp, Chlamydomonas sp.
  source 1..564: mol_type = other DNA; strain = M2762
ccagcgcgcaggctggacaagctggacgcagccatcgcaaagctggtggacctgcacgcggtcatctacctggcgggtgatgccaccaaggccaaggaccagctctcccgcgtgctgcgcgacgtggagcgcaacacggtgtggcgcgacatggtcgccatggagcggcgggctgtgagcgccacggtggagggcaccaagcggccgccatggtggaagggctacacggagcacatggggctggtgctgagcgtggctgtcttcgcggtgcttctctcggtggagatattcgacgaggaagagaagaacaactgcctggcgctgctggcctttgtgtccatgctgtgggccacggaggccatcccgctcttcgccacctccatgctggtgccgcccctggtggtcatcctgagggtgctcgtggaccgcaccaaggacccccccgtgcgcctcacagcgcagcaggccgcgcccaccatcttccacgccatgttctcgcagaccatcatgctgctgctgggcggcttcgccatcgccgccgccctctccaagcatttcatcgcc

SEQ ID NO: 157 - DNA, 2316 bp, Chlamydomonas sp.
  source 1..2316: mol_type = other DNA; note = Chlamydomonas noctigama
atgaagttcacccaccagctcaagttcaacacagtgccggaatggagggaccattacatccactacgcggcgctgaagaaaatcatctacgccatcgccaaagccgaggctgacgagcatcagcatccagcgggccatgacgacgaacacctgggcgtcgcactgctggataaggttgaggccactgaggagtacctgatcaagagcctggacaaggagctggctgaggtcatcaagttctacatggccaaggaggcggagatcctgggcaagctggagcagctggacctggaggtgcacagcctggagcagcgcagtgctctgggcacgacgctgcggtcgacgtcgatgcccctgcccagcgatgctgtgcctgtgatccttgaagaagatgacctgtctcgcaccgagtcggtgcgtgcctcccgcaccgagttctggcgcaccaacagccgcagcctcaagcccacctccagagccctcatcaaggacagcggcaagatgaagcagcgcatcatagacctgtacagctctctgcacgacctggcggacttccttaacttcaacaaggagggcttccgcaagatactgaagaagcatgacaaggtgaccagcagcaacctgaaggaccgctactggagggtggtggaggacaagtaccccagcaagaaggcagaggtgctggagcaggccatggacaggctgaccgaccagtttgcagtgctgtacctgcagggtgacacagtgaaggccaaggacaccctgggcagggtgctgagggagcagatcaaggttgagcgcaacactgtgtggaaggacatggtggccatggagcgccgcacagtggcagctgtcatcaagccgggtgcagcggagcccaagaaggtgtccttcttcgccaagcaccactcccggatcatgctgctgctctccgtggtggtctttgcatcgctgctgtcggtggagatcttcccggagcctgagaagcagaactgccttgccatgctggtgtttgtgtccctgctgtgggccactgaggccatccccttgtatgccacctccatgctggtgccccccctggtggtgctgctcaaggtgctggtggaccgcagtcacgaggagcccatccgcatgacagcgcagcaagcggcccccaccatcttccatgccatgttctcccagaccatcatgctgttgctgggtggctttgccatcgccgccgccctcagcaagcacttcatcgccaagcagctggccatcgccgtgatgtcacgcgtggggcgcaagcctcacaacgtgctgctggcctccatgttcgtcgccacctttgcgtcgatgtggatcagcaacgtcgcggcgccggtgctcaccttctccatcgtcatgcccatcctcaagaccctggagaccagcagtgcctttgccaagtccatggtcatgggcatcgccctggcctccaacattgggggcatgacctcgcccatcagcagcccccagaacatctttgccatcgagcgcatgtccatggacgggcagccccccagctggctctcctggttcgcagtgtcactgccggtgtccagcgtgtgcatcatcctgtgctggctgctcatcctggcagtgtaccagccctggcgcagcgtcagtgacgtgcgccccctcaagcccaacaccgaccccatgaacatgacacaggtgtttgtgattgtgatctccatggcaacggtcggcctgtggtgcgccaacacagccctgcagtcgtacactggggagatgggtgtggtggcgatgctgccgctggttgcgttctttggctttggagtgctcagcaaggatgacttcaatggcttcctgtggaacgtggtcatgctggccatggggggcctggctctgggggaggctgtcaagagcagtggtctgctgcagtccattgctgaggccattaaggaggtcactgatggatatgacttgtaccaggtcctgctggtcttctgtgtcatgatcctcgtctgcaccaccttcatcagccacaccgttggtgccatggtgatcctgcccatcgtgcagaaggtcggggaggacatgcctgggccccaccccaagctgctggtcatggccgctgcccttatgtgctcaggtgccatgggcctgcccgtgagtggcttccccaacatgcaggctgtgtccctggaggactccactgggcagaactatgtggacaccctggacttcctcaaggtcggtgtgcccggctccgtactggcataccttgtcatcgtatcacttggctacacgctgatgctcctggtccgcttt

SEQ ID NO: 158 - DNA, 627 bp, unidentified
  source 1..627: mol_type = other DNA; note = Carteria crucifera
caggtgtatgtgatcgttgtgagtgtggtgactgttgtgctgtggtgcctcaacagtgccctacagaatgtgacgggtgagatgggagtcattgccatcatacccatggtggccttctttggcactggggtgctcagtaaggatgacttcaacggattcttgtggaacgtggtgatgttggccatgggcggtctagccatgggagaagcggtgaagagcagcgggctgttggcagctattgctgagggcatcaaagaattggtggcagggatggacttgtgggaggtgctagcgatattctgcagcctcatcctggtctgtaccaccttcatcagccacacggtgggggccatggtgatcttgcccatcgtgcagtcagtgggggagatggcgctgggccaccctcacccccgcctcttagtcatggggtcagcgctcatgtgcagtggcgccatgggcctgcccgtgtcaggcttccccaacatgaacgcagtggctctggaggactccactggggtcaactacgttagcactgtggacttcttgtgggtgggtatccccagcagcatctttgcttacgtggtcattgtgacagtcggctacttcctcatgctcatggtcagattc

SEQ ID NO: 159 - DNA, 1335 bp, Volvox aureus
  source 1..1335: mol_type = other DNA; strain = M2242
tgcttggcgttgctggtgtttgcatctttgttgtggtcactggaggccgtgcctctgtacgtaacaagtatggctctaccttttctgattgtcgccatgggtgtccttgttgaccacccaaatgattccaaggatccacctaaacgcctcacaccgcagcaagctgcaccagcgatcttccatgccatgttctcacagacaatcatgttacttcttggtggtttctcgatatcagccgctctgtccaagcacgccatcgcaaagcaggtggctgtggctattttgtctcgtgttggaagaaaaccacgcaacgtactgcttgctgcaatgtttacagccacattcgcatcaatgtggatttccaatgtggcggcaccagtactctgttttggcctaattcaacccatcctgaggaccctggaccctggacatccttttgcgaagtccctggttatgggcattgcgctggcttccaatgtcggaggaatgacgtcacccatatcttcaccccaaaacatttttgcaatagagcgcatgtcgatggatggacagccgcccagctggctctcttggttcgctgtagcaataccagtttccatcacatgcaattttctctgttgggccttactccttctggtctaccaaccaggacgagctttgggtgaggtccgaccgttaaaacccaacacggaccccatcaacgggacacaggtgtacatcatcgttgtgtcgatgttgactgtggcagcctggtgtgcgaacacattcctccagaggtatactggcgagatgggcatcatcgccattgtcccattggtagcgtttttcggtttcgacgtactgaataaggatgatttcaactcgtttctctggaacgtcgtcatgctggccatgggcgggttgtctctaggtgaagcagttaagagcagcggtttgcttgcagcgctcacaaacagtattagtgaactggtaacaggcttcactatgtttcaagttaccctcatattctgtggcctggtgctgctggcgacgacgtttatcagccacacagtaggggctatggttatcctaccaatcgtgcagagtgttggagagagtatggcggggacaccacaccctaagctgttggtcatggcatcggcgctcatgtgctctggtgctatgggcttgcctgtcagcggatttccgaacatgaacgctgtcagcttggaggatagcactggaaatgcgattgtcagtactaaggacttcctgtacgtcggtgtgccttcatcggtgatggcttatggcataatcgtcagtctagggtatgtgctcatgctgttggtaggtatg

SEQ ID NO: 160 - DNA, 2610 bp, Phacotus lenticularis
  source 1..2610: mol_type = other DNA
atgaagttcacgcatcagctcaagtttaactcggtcccagagtggcgggaccagtatgttgactacgcccacctcaagcgcttcatctatgccattgcccgcgccgagcaggatgacatccagcagctgcacgaggtgcacgacaccacgatgcctctgctgccccacaccgtcaccatgggtcatgacaaggtggaggccacggaggagaacctgcgccaggcgctggacaaggagctgcagcgcgtcatctccttctacatggccaaggaggcggacatcttggccaaggtgacggcgctggagctgggcatccatgcgctggagaagctgcccgcccggggggtcagtctggagctggaccccacccggcagggcagccaggtggcggctgggggcgttgcgggagggggggcccccccaggggggcgccacgtccccctactgcagggcgccccctcggtcacgcgcgagggctcgggcggcattgcgcactccatctccccccagcagtcctcctcctccccccagctgggcggccccggcagtggccgcaccgccggcaccgggggcagtcagtccgcctccccccagccctcgggagccatgcacgggggggacctggaggcccccctgcaggggggcgaccacaccggcaagacctccccccactggtcacgcgccgcccgcgtcgagttctgggggcgggcccagcccgcgcaccgcttcaccgggggagcctccttctcagccgcctccttctcggggcccttcgtgcgcgacatgcaggcccacaaggagaggctgcgcccccagttcagcgacctctatctctccctgcacgacctcctgggcttcctcaggctcaacaaggagggcttccgcaagatcatcaagaagcacgacaagatgaccagcagcaacctgagggagcagtactggccgctgctggaggccaagtaccccatccagagggccgagctactggaggccaccatcgcctccctggtggaccactacgccgtcatctacctggggggcgacgtgggcacctccaaggcccacctggacaaggtgctgcgtgaccagatccaggtggagcgcaacacagtgtggagggacatggtggctcaggagaggcgcaccacagcggccgtggtggccaccacgtacaagcagaaagtgtgggccaaggtgacgccccacatcgcgctggtctccagcgtcgccgtctttgccctgctgctgtctgtggaggacctgttccccgaggcgcccgagaagcagaactgcctggcgctcctaatcttcgtgtccatgctgtgggccaccgaggcggtccccctctacgtcacctccctggccatccccctgctggcggtcacgctgagggtgctggtggacaagaccaccgaccccccccagcgcatgccggcgcagcaggccgcccccgccatcttccactccatgtgctcccaggtgatcctgctgctgctgggtggctcggccatcgcctcggccctcaccaagcacttcattgccaagaagctggcgcaggtggtgctggcccaggccggccgccagccccacaatgtgctgctggcgctcatgctggtggcaaccgtggcctccatgttcatctccaacgtggccgcccccgtgctctgctggtccctggtggagcccatcctcaagtcctttgacgccgacaaccccttctccaagtcgctggttatgggcatcgcgctggcctccaacattggcggcatgacctccccaatctcctccccccagaacatctttgccattgagcgcatgggcatggacgggcaccccccctcctggctctcctggttcgcggtggcgctgccagtctccttcatctgcatcctggtgtgctggggcctcatcctgggggtgtaccgcccctggtccaaggtggcggagctgaggcccctcaaggccagcgccgacaaggtcaccttcacccagatctacgtggtgctggtgacggtggtgacggtggggctctggtgctgcaacacgatgctgcagccgtacacgggcgagatgggcatcgtggccaccatccccatcatcgccttctttggctttggggtgctcaacaaggacgacttcctctcctccccctggctggtcatgacgctggccatgggcggcctggcgctgggcgaggcggtcaagagcagcgggctgctgctgtccatcgcacactccattggcgacctggtgcaggacctggacctcttcaccgtctgcgtcatcttctgtggcttagtcctggtctgcgcctccttcatcagccacacggtgggcgccatgatcatcctgcccatcgtgcagtcggtgggcgagcagatgccgggcccccaccactccaagctcctggtcatggcctcggccctcatgtgctcgggggccatgggcctgcccgtgtcgggcttccccaacatgttcctcatctccaaggacgacggcaccggcaagaactacgtcaacacgctggacttcatcaaggtgggcgtgcccggctcagtgggcgccttcttcgtcatcgccaccgtgggctacgtcctcatgctcatggtg

SEQ ID NO: 161 - DNA, 1896 bp, Stephanosphaera pluvialis
  source 1..1896: mol_type = other DNA
aggagacagagagtgcgcttctgggcttctctagacaccagggcagagctccgggacttgcggctggtgcggggcatgatgcgctaccgcttcaacgacatctacacaacactgaatgacctcatggaatacatcatgctgaaccgcgaggggctccgcaaggtggtgaagaagcacgacaagctgaccacaactgtggcgctcaaggaatcctactggccgactgtggaccaacagctggcgctgagcaagagggatgcaatggcccagcagatagagcagctggtggacctgtacgcggtgatgttcacagcaggggacgtggacgctgcgaaggagctgctgagcaaaaacttgcgcgaacacatcaaggtggagcgcaacacggtgtggcgggacatggtggcccttgagcgtcgcacagtggctgccacagtgcagcagaccacaggcaaggccgcagccaaattgcagaggtacagggagcccttgtgtctgctgctgtcccttgctgccttcttcgccctgctgcgggcagccccctttgctgagccagagaagaaccagtgcctggccctgctggcgctgtgctccctgttgtgggcaacagaggcagtgcccctgttcgccactgccctggccatcccccctttggttgtggtcatgcgggtgttggtggacaggagtgaccctgcagcgccacacaggctgacgccccagcaggccgcccctgccatcttccacgccatgttctctcaggtcatcatgctgctgcttggcgggtttgccatcgctgccgcactctccaagcacttcattgcaaagcagatggcagtggcagtcttatcccgtgtgggacgcaagccacacaacgtgctgctggctgccatgtttgtggccacctttgcgtccatgtggatcagcaacgtggccgcccctgtgctttgcttctcccttgtgcaacccatcctgcggaccatggacgtgacaacgccatttgccaagagcctggtgatgggcatcgcgctggcatcaaacataggtggcatgacctcgcccatctcctcgccccagaacatttttgccatagagaggatgggtatggatgggcacccgcccagctggcttgcatggtttgcagtagcccttcccgtcgccatcatcagtaacctgctggcttggggcttgttgctgctggtgtaccggccatggacacacaccacagaggtccgccccctcaagcccagctccgaccccatcaacctgacgcaggtgtatgtgtgcctggtgagtctggcaacagtgggtctgtggtgcgccaacacagcactgcagaagtacactggggagatgggtgttgtggcagtgctaccccttgtggccttctttggctttggtgtgctgaacaaggacgatttcaatggcttcctttggaacgttgtcatgttggccatgggcggctcagcactgggtgaggctgtcaagagcagcgggctgctggtgtccatcgctgagagcatcaggcagctggtggcaggcatggacctgtggatggtgacggtggtgttctgcctggcagtgctgttctgcaccaccttcatctcacacacagtgggagccatggtcatcctaccaattgtgcagagcgtgggggaggccatgcccgggccgccccactccaagctgctggtcatggcctctgcgctcatgtgctcaggtgctatgggcctgccagtcagtggcttccccaacatgaatgcagtgtcactggaggatgctacagggcaaacgtatgtcagtgccagtgacttcattgcagtgggagtgccatccagcgtagcagcatatgcagtcatagtcacagtggggtacagtctgatgctgctggttgggttc

SEQ ID NO: 162 - DNA, 2442 bp, Chlamydomonas sp.
  source 1..2442: mol_type = other DNA; note = Chlamydomonas eustigma
atgaagttcactcatcaaattaagttcaacagtgttcccgagtggagagaccactacatcgactatgcccacctgaagaaaatcatatatgcaatcgcaaaagctgaggctgatgagcagcagcaacaccatctagatgaggaacatcctcttcttaccagacaacagactgctcatggagagaaggttgaagctactgaggaggctttgatccaggcacttgataaggagcttgccaagattatcaaattcattatggccaaagaggctgagaccctggggaagctagctcagctggatctagaggttcacagtcttgaggcccagcgggttggaagtatgttcacacctcccattgtgaacaggttcacatcacttcaagatgcagggaatacacgtcttggaggctcactgccagatccccaaaaagatggttttgaaactctaggactagccgacagaagaccctctgaggttatggaggaggctgtacgccctgatctggaagggggtattggctcaaactctttccgtgcctcccgagtccatttttggcactcaaacagtctgcctgctaccactcgcactggtgctcgagtactggctaaggacagtgctaagatgaaaccaagaatcacggacctgtttgttgtgctgcatgacttgaagaactatctgtccttaaacaaggagggcttcagaaagattttgaagaagcatgacaagatgactagcagtaatctgaagagcaggtattggtgtatcattgaggaacagtaccccagcaagaaagaggagggcatcatgcaagctatcaacaagctagtggatctgtatgctgtgctcttcctgaagggtgattttgagaaggcatcaagcgtccttaatcgtgtgctaggagaacagattaaggtggagaggaacactgtgtggagggatatggtggcaatggagcgcaagactgtgaatgctgcagttcataagccacaaggtgtggccactcgtgtcacctggctgcagcagaacatgaagcacatcctgttgatgcttgccgtcctgacctttgccacacttctgacagtgcagacctttgaagagccagagaagaacaactgcttggctatgctggtgtttgtgtccatgctgtgggccactgaggccatccctctgtttgccacctccatgctggtgccacccttagtggtcatcctcagggttatggtggatcacacaaagtcacctccagagcgcatgcctgcgaaggatgctgcacctgccatcttccactccatgttctcacaggcaatcatgctcttgctgggtggtttcgctattgctgctgccctgagcaagcactacattgccaagcagctagctatctcggtcatgtccagggttggacgtaaaccccagtttgtgatcctggccgccatgtgtgtggcagccttcgtgtccatgttcatcagcaatgtagctgcgcctgtactcacgtactctattgtcatgcccatcttgaagaccttggatacaggctgtccctttggtaaggccttagtgatgggtattgccctggcatcgaatgtcggagggatgacttcccccatcagctctcctcagaacatatttgcgatccagctcatgtcaaacgacagcaacccgcccagctggctggcctggtttgccatctccctgccagtatcggctctttgtgtcttgatgtgctggtccctgatcttgattgtgtatcagccttggcgccgcgtggccgaggtgcgtcccctaaagcccagcactgatcccatcaacggcacccaggtctatgtcatcatcatctccctggctacagtggctttgtggtgtgccaacaccgtcctcacaccgtacactggggagatgggggttgtagcagtgttgccgctggttgccttctttggtttcggggtgttgagcaaagaggacttcaatgggttcttgtggaatgttatcatgctggccatgggaggcatggctgtgggagaagctgtaaaaagcagtggcctcctccactccattgcgctgggcatacaggatctgacctcaggtcttgatctcttccaggtcatgatcatcttctgcctcctggttctcatttgcacaaccttcatcagccacacggtgggtgccatggtcatcctgcctatcgtccagagtgtgggagagtccatgccgggcacagcgcaccccaaattactggttatggcaaccgtcctcatgtgctctggagctatgggcttgccaatcagtggttttccaaacatgcaagctgtttccctggatgatggcatggggcagaactatgtcagcaccatagacttccttatggtgggagtccccagctctgtgctagcttacttcgtcatagtctcagtgggatactctctaatgctcctagtgcgcttttga

SEQ ID NO: 163 - DNA, 2430 bp, Chlamydomonas sp.
  source 1..2430: mol_type = other DNA; note = Chlamydomonas incerta
atgaagttcactcaccaacttaagtttaatagtgtgccggaatggcgagaacactatatacagtatggacacctaaagaaatatatttatgcgcttgcaaagagggaagcagaccttcaagctggcggccaggaagaggaggcgcttctcgccccgctgctgctggaagcggggcgcgatcagggccccacggaggagggcttccagcaggagctggatgcgcagctcgcagccacgttgagcttcttcgcggtgaaggaggctgacctgctcgccaaggtgtcggcgctggagctggacatccagagcctggagaagatccccaaccgcgccgaggcctccaccctggcgcgcatgggcatgggcatgggcggcagcgccagccccggcggccccatgagcagcccgcgcgccgccgccgccgccgccatgtcggccgtggcctcgctggtcagccacagcccctccacactggacctggcgcgcatggtcaacagcacgccgcccgaggaccaccgcaaggtgcgggtcaagttctgggagaacccgccgcggcacctgttcagcaccaacctcagcgcgcgccgtgccaagctgcaggcgcgcttccaggacctgtacatctcgctacacgacctgcgcgagttcctgcacatcaacaaggagggcttccgcaagatcatcaagaagcacgacaagctgacgcgcgccgtggacctgcgcgcgcgctggtggcccaacgtggaggcgcacctggcgcccgccgccaagcaggcggagctggacggtgccatagccgcgctgaccgaccactacgccgtgctgtacacgcgcggcgacgtggcccaggcggaggagcagctgtcgcgcgggctgcgggagcacatcaccgtggagcgaaacaccgtgtggcgcgacatggcggccatggagcgcaagtacgcggcggtgtcggtgaagcaggcggcggcgcccggggcgcgagtcacgtggctgcgcacgcacgcgcgctggctgaagctagcgctgagcgtggcggtgctggtggtgctggccaacgtggaggtgtggccggggcccgagaacgagccgcgcaacaactgcctggcgctgctggtgttcgcgtcgctgctgtggagcctggaggccgtgccgctgttcgtgaccagcatggcgctgccgctgctgatcgtggccatgggcgtgctggtggaccgcagcaaggacccgccgcagcgcatgagcccgcagcaggcggcgccagccatcttccacgccatgttctcgcagaccatcatgctgctgctgggcggcttctccatcgccgccgcgctgtccaagcacgccatcgccaagcaggtggctgtggccatcctgtcgcgtgtgggccgcaagccgcgccacgtgctgctggcggccatgttcaccgccaccttcgccagcatgtggatcagcaacgtggccgcgcccgtgctgtgcttcggcctcatacagcccatcctcaggacgctggaccccggccacccctttgccaaggcgctggtgatgggcatcgcgctggcctccaacgtgggcggcatgacctcgcccatcagcagcccgcagaacatcttcgccatcgagcgcatgagcctggacggcagcccgccctcctggctagcctggttcgcggtggcgctgcccgtggccgtggcggccaacttcgtgtgctgggggctgctgctgctgtgctaccagcccgacaaggccatcgccgaggtgcgccccatcaagcccaacaccgaccccatcaacggcacccaggtgtacatcatcgtggtgtcgctgctgacggtggcggcctggtgcgccaacaccttcctgcagcgctacacgggcgagatgggcgtgatcgcggtggtgccgctggtggcgttcttcggcttcgacgtgctcaacaaggacgatttcaacagcttcctgtggaacgtggtcatgctggccatgggcggcctcagcctgggcgaggccgtcaagagcagcggcctgctggcggcgctggcgctcaccatcagcgacctggtcacggggctcagcctgtggcaggtggccaccatattctgcggcatggtgctcgtggccaccaccttcatcagccacaccgtgggcgccatggtcatcctgcccatcgtgcagagcgtgggcgaggccatgcccggcacgccgcaccccaagctgctggtcatggcggcggcgctcatgtgctccggcgccatgggcctgccggtgagcggcttccccaacatgaacgcggtcagcctggaggacagcaccggcaacgccatcgtgggcaccggcgacttcctggcggtgggcgtgcccagctccgtgttcgcgtacggcatcatcgtctcgctcggctacctgctcatgctggcggtgggcttctag

SEQ ID NO: 164 - DNA, 2262 bp, Chlamydomonas sp.
  source 1..2262: mol_type = other DNA; note = Chlamydomonas schloesseri
atgaagttcacacaccaactcaagtttaatagtgtgccggaatggcgagaacactatatacaatatgggcatttgaaaaaatacatttatgcgcttgctaagaaggaagcggacctgcaagctggcggccacgatgacgaggaggcgctgcttgctccgctgctggaagcaggacgtgatcagggccccacggaggagggcttccagcgtgagctggatgcgcagctcgcggccacgctgagcttcttcgcggtgaaggaggccgacctgctggccaaggtgtccgcgctggagctggacatccagagcctggagaagatccccaaccgcgccgaggcctccaccctggcgcgcatgggcggccccggcagcgccatggccagccccggcggcggcggccccatggccagcccgcgcgccgccgccgccgccgccatgtcggcggtggcgtcgctggtcagccacagcccctccaccctggacctggcgcgcctggtcaacaacacgccgccggaggaccaccgcaagatccgcgtcaagttctgggagaacccgccgcgccacctgttcagcaccaacctcagcacgcgtagggctaagctgcaggcgcgcttccaggacctgtacatctcgctgcacgacctgcgcgagttcctgcacattaacaaggagggcttcaggaagatcatcaagaagcacgacaagctgacgcgcgccgtggacctgcgcgcgcgctggtggcccaacgtcgaggcgcacctggcgcccgccgcaaagcaggcggagctggacggagccatcgcgcagctgacggaccactacgcggtgctgtacacgcgcggcgacgtggcgcaggcggaggagcagctgtcgcgcgggctgcgtgagcacatcaccgtggagcgaaacaccgtgtggcgcgacatggcggccatggagcgcaagtacgcggccgtgtcggtgaagcaggcggcggcgccaggggccagggtcacgtggctgcgcacgcacgcgcgctggctcaagctggcgggcgccgtgcttgtgttcctggtgctggccaacgtgcaggtgtggccgggcgccgagaacgagccgcgcaacaactgcctggcgctgctggtgttcgcgtcgctgctgtggagcctggaggccgtgcccctgttcgtgaccagcatggccctgccgctgctgatcgtggctctgggcgtgttggtggaccacaccaaggacccgccgcagcgcatgacgccgcagcaggccgcgcccgccatattccacgccatgttctcgcagaccatcatgctgctgctgggcggcttctccatcgccgccgcgctgtccaaacacgccatcgccaagcaggtggctgtggccatcctctcgcgtgtgggccgcaagccgcgcaacgtgctgcttgcggccatgttcactgccaccttcgccagcatgtggatcagcaacgtggcagcgcccgtgctgtgcttcgggctcatccagcccatcctcaggacgctggacccgggccacccctttgccaaggcgctggtgatgggcatcgcgctggcctccaacgtgggcggcatgacctcgcccatcagcagcccgcagaacatcttcgccatcgagcgcatgagcctggacggccgcccgccctcctggctggcctggttcgcggtggcactgcccgtggcggtggcgtgcaatttcgtgtgctggggcctgctgctgctgtgctaccagcccggcaaggccatcgccgaggtgcggcccatcaagcccaacaccgaccccatcaacggcacgcaggtgtacatcattgtggtgtcgctgctgacggtggcggcatggtgcgccaacaccttcctgcagcgagaagtgcacaaatccatttatgctacaacgggcgagatgggcgtgatcgcggtggtgccgctggtggcgttcttcgggttcgacgtgcttaacaaggacgacttcaacagcttcctgtggaacgtggtcatgctggccatgggcggcctcagcctgggcgaggccgtcaagagcagcgggctgctggcggcgctggcgctgtccatcagcgacctggtcaccggcctcagcctgtggcaggtggccaccatcttctgcggcatgagcgcagccaagctctggagtccaccggggggacgctccaaccaacctggaccacgccagcaacctcagaagggttactgctggtacaacaacgcagggcccagcgggctcaccaaccactag

SEQ ID NO: 165 - DNA, 2313 bp, unidentified
  source 1..2313: mol_type = other DNA; note = Chromochloris zofingiensis
atgaagttcagtcagacgttgaagttcaataggagacccgattgggagatccactacatcaactatgcccatttgaaaaggctaatcacgaaggtgcagcaggctgaatttgcggagcagaacaacctgccattgcacttcggggatgaggaagcaggtgtcagatcccctttgctcagccagacgtccttcaaccgtcagcagtctgtgtcagcagctcttactcgccagcagtctttcaccatatcagcagcacagtgcgatgaagcattcatcaaagcgctggacagcgaattggccagaatcatccagttctacatgcgcaaggagagtgagttgctggcaaggtttgagtctgcggctctaaggatccacagcattgaagggccagcactgccaggaccagctgccttggatacagcacaaaggatccagttttggtcacaggacacaaaggagattgcactggagcgtgagaagcttcgctctgagatgaccgacctgtatgagcagctgcatgcgctcagcaaatacttagaactgaacttcacaggcttcaggaagatcttgaaaaagcatgacaaaatgacatcccagaatcagtacaaggactcctacatgccgattgtggaagccaaactcccattgaagaaccgtgaaatgatttctggtgtcatcaacaacctggtggagatgtatgcggtcgtgtgtacacgcggtgatgtcaatcgtgcgcaagctgagctcaagcgcaagttgaaagatgaggtcgcgtttgaacggtctactgtgtggagagacatggtggctatggaacgaaggggtgcttctgtggctgtacatgaagcttctagtttggctgatcagccaaagaagccccgttggtggcaagcacatagacaactactgctggtgacattgtgcgttactgtgttcgctgtcctgctgtctgtacccattttccagcagccggagaaacagaattgcttagctctgctggcgtttgtgtcattactgtggtgtacggaggccatccccttgtttgtgacatccatattggtgcccttgttgattgtggtgctgcgtgtgttggtagatcgcagcgcagatcctcccagacgcctaccgcctcaggaggctgccccagcggtgtttcatgtcatgttctcacaggtcatcatgctcctgctgggtggctttgctatagctgcagccctcagcaagcactttattgccaagcagctggctgtagcaatcctcagtcgggtgggcaggaagccgcagtatgtgctgcttgccaacatgttggtggcaacctttgctagtatgtggatcagtaatgtggctgcaccagtattatgcttctcacttgtacagcccattctaaggaccctgtccccaagtcacgcgtttgctaagagcttagtcataggcatcgcattggcatcgaatctggggggaatgacgagccctatctccagcccccaaaacatttttgcaattgagcggatgagcatggatggcaaccctccaagttggttgagctggtttgctgtcgcactgcctgtgtctgtgctgggcaatctgctgtgctggggcttgattctgttggtgtataacccaggagctaccatcaaagaggttcgccccgtgaaaccgccagaagatccgctcaacggcacccaaatctacgtcatcctcgtcagcgtggctactgttggtctatggtgcttcaattccttcatacaacatgtgacaggagagatgggtgtgctggcgatcctgccattggtagcattctttggctttggagtgctggataaggacgactttaatggattcctttggaatgtggtcatgctggctatggggggcttggcactgggtgaagctgtgaagagttcaggactgctgctaacaatagcaacgggtattcaggactttgtggctggtcttggcctatggtccgtgctggctgtgttctgcttcctggtgctcatctgcaccaccttcatctcacatacagtaggcgccatgatcatactgcctatcgtgcaatctgtgggagaaacaatgagcggcacgccccatcccaagctattagttatgggctctgcgctcatgtgctcaggggctatggggctgccagttagtggcttccctaacatgaatgctgtggctttagaagaccccaccggccaaaactacgtcaacaccattgatttcttgaaggtgggtgtacctggttccatcatggcgtatggagtgattgtcagtttggggtatgtgctgatgatagctgttggcatgtaa

SEQ ID NO: 166 - DNA, 2406 bp, Coccomyxa sp.
  source 1..2406: mol_type = other DNA; note = Coccomyxa subellipsoidea
atgaagtttggcgcggagagggcaggccacgcgctgctgagctggctgactgctgcctggctgtggcttctgcaagcctgggaggtggtcgcagagtggggccgccagtgctggggggctcttttgcatgcctggcattatatcgccagcgctgtcatgcaggcagtgcactggcaaacagagaatcgaatcgcagatcttggacgtattccagaggaggtaggaggtgacctggacaggacaatatccctggctcttgaagagggcggggacgatatcaagggcgcatttgacagcgagcttaaccgcattactactttccacaaaaagaaggaagaggagcttctgggtgcagtggacaagcttggggaggaggtgagcagtgctgtggagccaagtgcacagcagagcgctcctgatgcgagctcgccgctgcttgggacttccaggaatgcggaggcactgtactggggccaggacactgttgctgtgcgcattgcgcgggagcagctaagggagacctttcaggagctgtatgtggagatccaggggctgatagattttgtggaggtgaatcgtacaggcttcaggaaggccctgaagaagcacgacaaggtgttgggcgcgcttgggcacccaaagatgcagccaacgtacatgcctaatgtcgaggctgccttccctgagaagaaccgcctgcgtgtgtcagaagctcagaagcagctggtggagctgtacgctgtggtgtgctgccacaacaacctgctgctggcccagctggagctcaaggcacagctgcgctcccagctcaagcttgagaggacgacagtctggaaggacatggtggagaaggagcgaaaggagaacgctgccacggtcgacgacagcggcgctgagtccaagccctggtaccgcagcagcctcttcatgatcgctctctcttgcgtcgtctttgccgtgctcctcagcgtgccgatcttcgaggagcgagcgaagcagaattgcctggcgctgctgggatttgcctccatgctgtggtgcacggaggccctgccactctacgtcacctccatgctagtgcccctgctggctgttgtgctcagagtgatggtggacgacagcgggaagcacccggtcaggaagagcgcacccgacgctgcggacgccatcttcaaagccatgttctcacaggcgagttcacagctcttcatctcccctcattgcacaattgagaggcacgttgacggcctgccctcataccccactacaatcatgctgctgctggggggattcgcgatagctagcgccttcacaaagcacttcatcgccaagcgcgtggccgtctgggtgctggggaaagtcagcgccaagccgcacgcggtgctgatagccaacatgttcgtggccaccttcgcatccatgtggatcaccaatgtggccgcccccgtgctgtgcttctccgtgctggacccaatcctgcgcacgctgccctccggccattcttttggcaaagctctggtcctgggcatcgcgctggcgtccaacctgggaggcatgacaagcccgatctcatcgccgcagaacatcttcgcgatccaggagatgggccgagatggcgagccgccctcctggcttgcctggtttgcggtggcgctgcccgtggcgtgcgtgggtaactttgcctgctggggatttctgctgctggcctaccggcccggccgcaccctcaaggaagtccgccgtatgcccttcagctcggacccgttcacgtggaagcagatctacgtggtggtgatcagcctgggcacggtggggctgtggtgcgccaacaccgcgctctccaagttcaccggccagatgggcatcgtggccatcgtccccatggtcgccttctttggcttcggcctcctctccaaggatgacttcaacaaccagctgtggaacgtggtgatgctggcgatgggcggctccgcgctcggcgaggccgtcaagtccagcggacttctctcctccattgcgcactccatcgaggacgtggttgccggcatgggcgtctgggccgtcttcgccatcttctgcgcgcttgtgctcgtggccacaaccttcatctcccacaccgtgggcgccatggtcatcctgcccattgtcagcgctgttggcgcgcaaatggaggagccccacccgcggctgctggtgatgggggcagctttgatgtgcagcggagccatgggcctgccggtgtcaggcttccccaacatgacagcctacgccaaggaggaccccaccggcaacccctggctatccaccatcgacttcttcaaggtgggcgtgccatgctcactggccacgtatggcctcatcgtgacagtaggctatggcatcatgaagttcgttctgggctggtga

SEQ ID NO: 167 - DNA, 2379 bp, Coccomyxa sp.
  source 1..2379: mol_type = other DNA; note = Coccomyxa subellipsoidea
atgaagtttggcgcggagagggcaggccacgcgctgctgagctggctgactgctgcctggctgtggcttctgcaagcctgggaggtggtcgcagagtggggccgccagtgctggggggctcttttgcatgcctggcattatatcgccagcgctgtcatgcaggcagtgcactgggtgagggggctcgaggaggtaggaggtgacctggacaggacaatatccctggctcttgaagagggcggggacgatatcaagggcgcatttgacagcgagcttaaccgcattactactttccacaaaaagaaggaagaggagcttctgggtgcagtggacaagcttggggaggaggtgagcagtgctgtggagccaagtgcacagcagagcgctcctgatgcgagctcgccgctgcttgggacttccaggaatgcggaggcactgtactggggccaggacactgttgctgtgcgcattgcgcgggagcagctaagggagacctttcaggagctgtatgtggagatccaggggctgatagattttgtggaggtgaatcgtacaggcttcaggaaggccctgaagaagcacgacaaggtgttgggcgcgcttgggcacccaaagatgcagccaacgtacatgcctaatgtcgaggctgccttccctgagaagaaccgcctgcgtgtgtcagaagctcagaagcagctggtggagctgtacgctgtggtgtgctgccacaacaacctgctgctggcccagctggagctcaaggcacagctgcgctcccagctcaagcttgagaggacgacagtctggaaggacatggtggagaaggagcgaaaggagaacgctgccacggtcgacgacagcggcgctgagtccaagccctggtaccgcagcagcctcttcatgatcgctctctcttgcgtcgtctttgccgtgctcctcagcgtgccgatcttcgaggagcgagcgaagcagaattgcctggcgctgctgggatttgcctccatgctgtggtgcacggaggccctgccactctacgtcacctccatgctagtgcccctgctggctgttgtgctcagagtgatggtggacgacagcgggaagcacccggtcaggaagagcgcacccgacgctgcggacgccatcttcaaagccatgttctcacaggcgagttcacagctcttcatctcccctcattgcacaattgagaggcacgttgacggcctgccctcataccccactacaatcatgctgctgctggggggattcgcgatagctagcgccttcacaaagcacttcatcgccaagcgcgtggccgtctgggtgctggggaaagtcagcgccaagccgcacgcggtgctgatagccaacatgttcgtggccaccttcgcatccatgtggatcaccaatgtggccgcccccgtgctgtgcttctccgtgctggacccaatcctgcgcacgctgccctccggccattcttttggcaaagctctggtcctgggcatcgcgctggcgtccaacctgggaggcatgacaagcccgatctcatcgccgcagaacatcttcgcgatccaggagatgggccgagatggcgagccgccctcctggcttgcctggtttgcggtggcgctgcccgtggcgtgcgtgggtaactttgcctgctggggatttctgctgctggcctaccggcccggccgcaccctcaaggaagtccgccgtatgcccttcagctcggacccgttcacgtggaagcagatctacgtggtggtgatcagcctgggcacggtggggctgtggtgcgccaacaccgcgctctccaagttcaccggccagatgggcatcgtggccatcgtccccatggtcgccttctttggcttcggcctcctctccaaggatgacttcaacaaccagctgtggaacgtggtgatgctggcgatgggcggctccgcgctcggcgaggccgtcaagtccagcggacttctctcctccattgcgcactccatcgaggacgtggttgccggcatgggcgtctgggccgtcttcgccatcttctgcgcgcttgtgctcgtggccacaaccttcatctcccacaccgtgggcgccatggtcatcctgcccattgtcagcgctgttggcgcgcaaatggaggagccccacccgcggctgctggtgatgggggcagctttgatgtgcagcggagccatgggcctgccggtgtcaggcttccccaacatgacagcctacgccaaggaggaccccaccggcaacccctggctatccaccatcgacttcttcaaggtgggcgtgccatgctcactggccacgtatggcctcatcgtgacagtaggctatggcatcatgaagttcgttctgggctggtga

SEQ ID NO: 168 - DNA, 1812 bp, unidentified
  source 1..1812: mol_type = other DNA; note = Symbiochloris reticulata
atgcaattgggcctgggcagggacgacatgcagaggctgtttgtcctgctgacggggctggagcgttacatcgatttgaacattgccggcttccgcaaggccctgaaaaagcacgacaaggttctggcagatgcagagagcggcaagctgaaggagacctacatgcccactgtgcaccgccagtgctgcctcaacaagaagcccatcctggagacattgtatgccatcgtgtgctgcgatgggaacaatgagatggctttgatagatctcaagcgccgtctcggcgagactgtgcaatttgaaagaaacacagtgtggaaggatatggtgcaaaaggaccgcaaaaggggcacgctgaaggtcgacgatgggctgatcggatcgtggtggcatcgcgcgcggcagccggcagcaatcgccatgtcactggcggtctttgttgtgcttttatatacgcccacgttcagagagccggagaagcgaaactgcctggcgctactggccttcacctcgctgctgtggtgcacggaggcgctgccgctgtacgtgacgagcatgctggtgcccctgctggtggtggtgctgcgggtgttggtggacggcagccagcacccgcctcagcgcctgtcctgcaagcaggccgcgccccacatcttccatgcgatgaactcccaggtgatcatgctgctgctgggaggcttcaccattgcggctgccctgagcaagcacgcgattgccaagatcctggccagctgggtgctgagcaaggtggggcagcggccgggcgcggtgctcatggccaacatgctggtggccacctttgccagcatgtggatctccaatgtggccgcccccgtgctgtgcttctcgcttgtgcagcccgttctgcgcacgttggatgccacccacagctttgcaaaaagcctggtcatgggcattgcgctggcatccaatctggggggcatgaccagcccaatcagcagcccacaaaacctgtttgccattgagcgcatgtccatggcaggccttccgccctcatggctgtcctggtttgcagtcgcactgcctgtggcttttctgggtaactttctggtctgcggcttgttgctccttgtctatcaggaccctcatttcaccgaggtccggccaatgcagcccatcaaggatccgatcaacggcaagcagatgtacatcattgcagtatctgtcggctcggtcacaatgtggtgcttcaacagcgtgctccagcaatggtttggggagatgggtatcatcgctatactgcccatgatagcattttacggctttggcatactagacaaggacgattttaacagcatgctgtggaatgtcgtgatgctggctatgggcgggctggcgctgggggaggcggtcacatcctctggcctgctgctgtccattgcggagcagctgcagcacctggttcagggcgcctcggtgtggcgcgtgctggtcatcttctgcggcctggtgctcgtggccaccacctttgtctcccacactgtcggcgccatggtcgtcctgcccatcattcagtctgtcggctcccagctgtcggatccccatccaaagctgctggtcatgggcgcagcattgatgtgctcaggtgccatgggcctgcctgtcagtggctttccaaacatgaatgctgtggccttggaggactccaaaggcatcaactatctcaccacaatagacttcttcaaggttggcctgctgagttccttgatagcctatggacttatcgtcaccctgggctatggcatcatgtactatggcattggctggtaa

SEQ ID NO: 169 - DNA, 2442 bp, unidentified
  source 1..2442: mol_type = other DNA; note = Edaphochlamys debaryana
atgaagttcactcaccagctcaagttcaattcggttcccgagtggcgggaacattacatacagtatgcgcaccttaagaaatacatttacgcgcttgcgaagaaggaggctgaccaccaagcggacggcgccgggactggtgatgtagagggcctgatcgccccactgctgcaggatggcggtcgcgcatcgggccccaccgaggagggcttccagcgcgagctggactcccagctggccgcgctgctgggtttcttcgcggtcaaggaggcggacctgctggccaaggtgtcggagctggagctggaggtgcagagcatggagaagatccccaaccgcaacgaggcctccaacctggtccgggcgagggggggcggcagcgccgccagcggcacgccctccccgggcgcctccccgcgcgcctccgccgctggcgccgcgctgtccgccctcagcggcctgctggcggcctcgccctccaccatggacctggcgcgcatggtggccgcctcgccgccagaggaccaccgctccgtgcgcgtggccttctggaagaaccccccgcgtcacctcttctcctccagcctgcagtcccgcgcggccaagctgcagagcaggttccaggacctgtacattgcgctccacgacctgcgcgagttcctgcacatcaacaaggagggcttccgcaagatcatcaagaagcacgacaagctgacccgctccgtggacctgcgcgcccgctggtggcccaacgtggaggcgcacctggcccccgccgccaagcaggcggagctggacggggccatagcggggctcacggacacgtacgcggtggtgtactgccgcggcgacgcctcctccgccgaggagctgctcagccgcggcctgcgcgagcacatcacggtggagcgcaacaccgtgtggcgggacatggcggcgctggagcgcaagtacgcggcggtcagcgtcaagcaggcggcgggggcggccaagcccagctggctgtggcgccacgcccgctggctgaagctgggctttgcgctggcggtgtttgggatcatgctgcagtacgaggtgtggcccggccccgagaacgccccccgcaacggctgcctggcgctgctggtgttcgcgtcgctgctgtggtcgctggaggccgtgcccctgttcgtgacctccatgctgctgcccctgctcatcgtgctgctgggcgtgctggtggaccgcaccaaggaccccccgcagcgcatgaccccgcagcaggccgcacccgccatattccacgccatgttctcgcagaccatcatgctgctgctgggcggcttcgccatcgccgcggcgctgtccaagcacgcaatcgccaagcagttcgctgtggccatcctgtcccgcgtgggccgccgcccccgcaacgtgctcctggcctccatgttcaccgccacgttcgccagcatgtggatcagcaacgtggcggcgccggtgctgtgcttcgggctcatacagcccatcctgcgcacgttggaccccggccaccccttcgccaaggcgctggtgatgggcatcgccctggcctccaacgtggggggcatgaccagccccatcagcagcccgcagaacatcttcgccattgagcgcatgtccctggacggccgccccccctcctggctggcctggttcgcggtggcgctgcccgtgtccatagcctgcaactttgtgtgctggggcctgctgctggccgtgtaccgccccgagcgggtcatcgccgaggtgcgccccatcaagcccaacacggaccccatcaacggaacgcaggtgtacatctgcgccgtgtcgctgctgacggtgggcgcctggtgcgccaacaccttcctgcagaagttcacgggcgagatgggggtggtggcggtggtgccgctggtggccttcttcggcttcgatgtgctcaacaaggacgacttcaactccttcctgtggaacgtggtcatgctggccatgggcgggctgtgcctgggcgaggccgtcaagagcagcgggctgctggcggcgctggcgctgggcatcagcgacctggtcacggggctgagcctgtggcaggtggcggtggtgttctgcggaatggtcctggtggccaccaccttcatctcgcacaccgtgggcgccatggtgatcctgcccatcgtgcagtccgtgggcgaggccatgcccggcacgccccaccccaagctgctcgtcatggcggcggcgctcatgtgctcgggtgccatgggcctgcccgtgtccggcttccccaacatgaacgccgtcagcctggaggacgccacgggcaacgccatcgtggccacgcaggacttcctgttgagcggtgtgcccggcagcattgcggcgtacggcatcatcgtgacgctgggtcaccacaccatggcactgctggcagccccctga

SEQ ID NO: 170 - DNA, 2289 bp, Enallax sp.
  source 1..2289: mol_type = other DNA; note = Enallax costatus
atgaagttcacgcatgtgctgaagttcaactccgtgcccgagtggcgggagtcgtacatcaactacccgctgctgaagaagcttatccttgctgcaagtaccgctgaatatcatgaagcgtacgagggcctggcgctcacgcaagatgaggaggcagggccacggtctcctctgctgtcagcccagcccagcctcagccgttcgttatccgttacgatgacacgcgagcagcgcgagaaggagttcctcgaggccctcgacaacgagcttgcgaagatcatacgcttctatctgaagaaggaggcagagatcagtgccaagtttgaggagctcagcatgatggtccatcatgcggaggggataccttctcctaccccagagcaaatggcagatggccacgacgtgacgacagcagcgcgtgtagcattctggtcccaggggggccgagcagtagcagctcagcgtgaaaagcttaaaacatcgctcgaagagctttatgccacaacgttcagcctcgctaactatgtagaacagaacaggactggctttcgcaagattctcaagaaacatgataagctagtctcgcacacaatgtcgagcaactatctacccattgtagaccagaagttcccagccagccacgcagccacccttcatcaccagctagaggcaatcacagcactgtatgcagtagtatgctgcaacggtaacttggagcatgccaacagcatactacgtaagcagcagcaggagcaggtgtcgttccagcgtaacagcatctggaaagatatggtgggccaggagcgacgagcagcaacagtacgagtgcaggacggcaaagaggtagagccagagtcctggttcacagcccaccggcaagcagtcatcctggcaatcgcactagcagtgtttgtcgtgctactcacagtacccatattcaagcagcctgagaagcagaattgcttggcattgctggcctttgcaagtatgttatggtgcacggaggccattccgctgtttgtcaccagcatgttagtgcctttcctggtggtggtgttgcaagtgcttgatgatgtgactcaagagccccctgagaggctgacacccaaacaggcggcacccagggttttccacaccatgttctcacagaccatcatgttgctactaggaggctttgccattgcagcagccctcagtaaacacttcattgccaagcagctcgcagtagcaatactctctagagtaggccgcaagccacatcacgtactcctggccaatatgctcgtggcaacatttgccagcatgtggatatccaatgtagcagcgcctgttctgtgtttcagcttggtacagcccatacttcgaaccctgccgactacgcatgctttctgcaagagcttggtcattgggattgcactggcgagcaacttaggcggaatgacgagtcctatcgccagcccccagaatatatttgcagtcgagaggatgggaatgggtggtacaccacctagttggctggagtggtttgctatagctctgcctgtcagctttctgggcaacctgctgtgctgggggctgctactgttggtgtacaagcctgggaaagatataaaggaggttcgtcctctgaagcccactgaggatcctttgactggcacacagatatatgtcatcgtcatcagcttggctacagtcacactgtggtgctgtaacagcttcctacaggaatataccggagagatgggtgttctcgccatcttccctctggttgcgttctttggctttggtgttttgaacaaggatgacttcaacggctttctttggaacgttgtgatgctggctatgggtggactggcactgggagaagctgtgcagagcagtgggttactgctggagatatcaaatagcatcagtcaccttgttgctggtcagagcctgtgggcagtccttgccatcttctgtggactagtgcttgtgggcacaacattcatcagccacacagttggagccatggtgatcttgcccattgtgcaagcagtggggcagcagatgccgggtggagatcattcaaagctgctggttatgggtgcagctcttatgtgttcaggtgccatgggcttacccgtaagtggcttccccaacatgaatgccgttgcattagaagatcccactggggcaaactatgtctacacaaaggacttcctattggttggggtgcctggcagtatcatggcatatggcatcatcatcagcgtagggtatctgctgatgttggcagtaggcttttag

SEQ ID NO: 171 - DNA, 2322 bp, unidentified
  source 1..2322: mol_type = other DNA; note = Mesostigma viride
atgaagttcgggaaggtcctgaaggacgatgccgtccctgattggattccgaaatacgtggcatacaagaagttgaagcgtgtcgttcaacggatggagttaacagtagagcaggaactgcaacaagccgcgagcaaacggggagcagcaggctcgtcagacgtaacctcccctcttgccaccaaggagacgttgctgcagaggaagagtgatgagtttatggaaggggtggaggaggaggtggccaaagtgaaccacttttacgacgagatggtctcggcgctccgctgcgacctggaggcctacgagaagcagctcgcggcgcagctcgcgggcggcaacaagaaggcgttccagaagatgtttgtgctggcgtccgacctgaatgcgtacatcacgctcaacagcacggcgttccgcaagatcatgaagaagcacgacaagttgacgggcctgcaccgcatggacgcgtttgtggcgcgcatcaagcatgaggggttcatggaggcaaaggcgctgagggagctatccgcacgcctcgaggcgatgatgtcgcccgacgcgctcgacagcctcaagcagcagtaccacctggagcgccagaagcggtccgagtccgcagggggctccaccggatccccagccaagcccacgcgcatcctcttctccatcgctgtcttcttcctcatcctggcgctgccccccttttggagcgcgcgcccggcgagcggcggcaacgatgacgggatcgctgacgtcagcgacggtgccggcgtcagcggtggtgttgcgtttggggttgattatgggtatgagggtgagccggcgtcgttgggcgcccagggaggcgtcggggaggccgcggtggcggcgcgtgaccggctcatgcgtgtactgtgggagcggcactatgcgagggatgaggcggcctcctcgagcatcggcgactacgtctctggcaacagtgcgtttggccccactcaggaggagcgcgcgcaccggtgctttgcgctgctcatcttcatcgcgtgcatgtgggtgctggaggcgctgccgtacttcgtcacctccctcatgatcccgcccctggtggtcatgctgaacatcatggcggacccgacggacaaggacaaggcactgtccgcgcccgacagctcgcgcctcgtcctctcgtccatgttcgaccacgtgctcatcctgctgctgggcgggttcacgctctccgccgcgttcgggcagtgcgcgtttgagctgcgcatcgcaggcgcgctgcagcgggcgctcggccaccgcccctggctcttcatgctcgccatcatgctcctctcgctgttcctctgcatgtggctgtccaacgtgaccgcgcccgtcctcatgctctcggtgctgctgcccatcctgcgcgacttcgaccacggcgggcggtaccctaaggccctgctgctcggcctcgcgtttgcctgtaacctgggcggcatggtcaccccgatcgcgtcgcctcagaacgcggtcgccctggtggcgctcgacgcgcagcactttaccatcaccttcttcgagtggatggcggtcgcgctgcccttctgcgtgctcctcgtcgtcgtcgtctgggcctacctcatcttcgcgctgcggccagacgacgtggtgtccatcccgccggtcatgtacaagacgacccccctgagcagcaagcacatctgggtgctcctcttctcgctggccaccatcggcctgtggtccaccctctccctcaccgtgagcgtgctgggggacctgggcatcatcgcgcttctcttcatggtgtttgcgttcggcacgggcgtgctgtccaagcacgacctgaactccttctcctggcacctgctgctgctgatcgccggtggcaacgtcctgggccgcgcggtgcagtcctccgggctgatccagatcgtagcgcagatcgtgacgccctacctgcacgacatcctgtgggttgcggcgctcgagctgctcgccttcatgatcatcatcaccacgtttgtctcccactcggtcgcggccatcatcatgatgcctctcatcgtggcgatcgggaaggagatatcacctctctccgccgaggtgctggtcctcctgtgcacgctcgcggacagtgccgcgatggcgctcccgatgacatccttccccaacgtgaactcgctgctcgtggaggatgactatggcgttccctacctccgagtcgtcgacttcatcaaagtgggtgcccccgtgtcgatcatggtggtgaccgccatcgccaccctgggatactccctggctgtgtttgtgctgcgcccatga

SEQ ID NO: 172 - DNA, 2376 bp, unidentified
  source 1..2376: mol_type = other DNA; note = Raphidocelis subcapitata
atgaagttcacccaccagctcaagttcaacgcggtgccggagtggaaggagcattacataaactaccccctcctgaaaaagatcatctacgcgacccgcgcggccgagtgccaggacgcgtacgacggcgtcggcggggacgaggaggcggccggcccctccgcctccggcggctcgctgctgcgctccccccgcaccagcctcagcggcggctcgctgcgcgcgccgctgctgcagggcgtgggcgggctgtcgctgtcgcggtcgggcagcgtcggcgcgcgcgcgggggactctgaattcattaaggcgctggaccaggagctggcccgcatcataagcttctacctgcggaaggagggggagctgacctcggcgttcgagtccctcaacctgcagctgcacagccgcgatggctgcgacgcggctgcgcccgccgcgggcggcgccggcggcggcggcggcggagccgcggggtttggcaccgcgccggcggcgcccgcggcgggcgccgtggacggcgcggcggccgcagaggcgggggaggccgccgccgccgcggcggtcccgcagtcgcaggcggagcggcagcggcgcgccgagttccagcggcgcaccgcctactgggctgccaacgaccgcggcgtggcggcggagcgggagcggttccggcagaagctggtcgggctgtttgtgcagctggacggcctcaagaagtacttggagatgaaccacaccgggttcaggaagatcctcaaaaagcacgacaaggagaccacgcagcaccagtacaaggacagctacatggccatagtggacgccaagctgccgctgcgcagcctcgaggggctcaaccgcctgatagagcggctcagggagatgcacgcggcggtgtgctgcaagggcaatctggaaaaggcggagcgggagctgaggagcgagttgcgggaggaggtcggtttcgagcgcaacaccgtttggcgcgacatggtggccatggagcggcgcacgggggcggtggtgctgcaggagcccgcccacggcatcgccgatgagtcgcgccaggagccgtggctgcgccgccactggcagccgctggcgctgtgcgtctcggggctcgcgtttgccgcgctgctggcggcgccgctgttcgagggcgcgccggagaagcggaactgcctcgccatgctcgcgtttgtgagcctcctgtggtgcaccgaggcgctgccgctgtttgtcacgtccatgctggtgccgctgctggtggtggtgctgcgcgtgctggtggacaggaccgtggagccgccggtgcggctggaaccccagcaggccgcacccgcaattttcagagtcatgttcgggcaggtcatcatgctgctgctcggcggctttgccatcgccgcggcgctgtcgaagcatttcatcgcaaagcagctggcggtcgccatcctctcccgcgtgggacggcggccgcgggacgtgctgctggcgaacatgctggtggccacgtttgcgagcatgtggatatcaaacgtggcggcgccggtgctgtgcttcagcctggtgcagcccatcctgcgcacgctgccgccgagccacccctttgccaagtcgctcgtgatcggcatcgccctcgcttcgaacctcggcggcatgacctcccccatctcctccccccaaaacatctttgccatcgagcgcatgagcatggacggccacccgcccagctggctggcctggttcgcggtggcgctgccggtcgcgtttgcgggcaacgtgctgtgctgggggctgatcctggcagtctaccgccctgggcagaagatccgggaggtccgccccctgaagcccccagaggaccccctgtcacccacccaggtttacgtcgtcgtcgtgtcactcgccaccgtggcgctgtggtgctgcaacagcctggtggcgggggtgacgggggagatgggggtgctggccatcctgccgctggttgcgttcttcggctttggcgtgctgtccaaggacgacttcaacggcttcctgtggaacgtggtgatgctggcaatgggggggctggcgctgggggaggcggtcaagagcagcgggctgctgctgacgattgcgcagtccgtcggccagcagctgcccggcccgccgcacgacaagctgctcgtcatgggcgcggcgctcatgtgcagcggcgcgatggggctgcctgtctcggggtttccaaacatgaacgccgtcgcgctggaggacccaacgggggtgaattacgtggacacgatcgattttttgaaggtcggcgtgccgggcagcgtgctcgcgtactggatcatagtgaccgtggggtatgggatcatgagggccgtggggatgtga

SEQ ID NO: 173 - DNA, 2196 bp, unidentified
  source 1..2196: mol_type = other DNA; note = Symbiochloris reticulata Africa
atgaagttcacgaaggagttgaaatataacgccgtggaagagtggcgcgcccactacatcaactatgccgctttcaagcggctcatatacggcgaagagaagcgcaaatttggcgataacgaacgcatggtgccgggaacgccacaggaagatgaccatcccactcaggagccactgctacaccagacagatgacaaagctttcatgagccttttggacagcgaactggctcgtgtgcacgaattttaccttgaaagggagcgagagcttggtggccagcttgacagcttgctgagccatgcgcgcactgtggaagtcaatgaacggcctgccaccccttcaacagagcacggccgcagatcttctgagggcagattacaccttgcgaggcggagcagttccagaatgcagggagcgctggcagatttgcaggcagaagccgtatcctcagagttctggtcccagaaccaggactttgctgtccaggctgcacgcgagcaactcagggacgacatgcagaggctgtttgtcctgctgacggggctggagcgttacatcgatttgaacattgccggcttccgcaaggccctgaaaaagcacgacaaggttctggcagatgcagagagcggcaagctgaaggagacctacatgcccactgtgcaccgccagtgctgcctcaacaagaagcccatcctggagggggcgctgcggaagctgcagacattgtatgccatcgtgtgctgcgatgggaacaatgagatggctttgatagatctcaagcgccgtctcggcgagactgtgcaatttgaaagaaacacagtgtggaaggatatggtgcaaaaggaccgcaaaaggggcacgctgaaggtcgacgatgggctgatcggatcgtggtggcatcgcgcgcggcagccggcagcaatcgccatgtcactggcggtctttgttgtgcttttatatacgcccacgttcagagagccggagaagcgaaactgcctggcgctactggccttcacctcgctgctgtggtgcacggaggcgctgccgctgtacgtgacgagcatgctggtgcccctgctggtggtggtgctgcgggtgttggtggacggcagccagcacccgcctcagcgcctgtcctgcaagcaggccgcgccccacatcttccatgcgatgaactcccaggtgatcatgctgctgctgggaggcttcaccattgcggctgccctgagcaagcacgcgattgccaagatcctggccagctgggtgctgagcaaggtggggcagcggccgggcgcggtgctcatggccaacatgctggtggccacctttgccagcatgtggatctccaatgtggccgcccccgtgctgtgcttctcgcttgtgcagcccgttctgcgcacgttggatgccacccacagctttgcaaaaagcctggtcatggtcgcactgcctgtggcttttctgggtaactttctggtctgcggcttgttgctccttgtctatcaggaccctcatttcaccgaggtccggccaatgcagcccatcaaggatccgatcaacggcaagcagatgtacatcattgcagtatctgtcggctcggtcacaatgtggtgcttcaacagcgtgctccagcaatggtttggggagatgggtatcatcgctatactgcccatgatagcattttacggctttggcatactagacaaggacgattttaacagcatgctgtggaatgtcgtgatgctggctatgggcgggctggcgctgggggaggcggtcacatcctctggcctgctgctgtccattgcggagcagctgcagcacctggttcagggcgcctcggtgtggcgcgtgctggtcatcttctgcggcctggtgctcgtggccaccacctttgtctcccacactgtcggcgccatggtcgtcctgcccatcattcagtctgtcggctcccagctgtcggatccccatccaaagctgctggtcatgggcgcagcattgatgtgctcaggtgccatgggcctgcctgtcagtggctttccaaacatgaatgctgtggccttggaggactccaaaggcatcaactatctcaccacaatagacttcttcaaggttggcctgctgagttccttgatagcctatggacttatcgtcaccctgggctatggcatcatgtactatggcattggctggtaa

SEQ ID NO: 174 - DNA, 2301 bp, Tetradesmus sp.
  source 1..2301: mol_type = other DNA; note = Tetradesmus deserticola
atgaagttcacccacaccctcaagtacaattccgtgcctgagtggcgcgagtcctacatcaactatagcctgctgaaaaagcttatcttagcggccagtactgcagaatatcatgaggcgtacgaaggcgtgcatcctgcagcagacctggaggatgctgggcccaggtcacccctgctatctaggcaggcaagtctgcaggcaagtctttccaggagtctctcagtcacgatgacgcgcgagcagcgcgaaaaggagttccttgagacattggacaacgagctggccaagatcatccgcttttacttgaagaaggaggcagagatcacagccaagtatgaagaagtcagcatgatggtgcagcatgccgagggcattgcatcgccaacaccagggcaggcagcagaagtctcggggttgcaggcagcacagcgcacagcgttctggtctcagagcagcaggccagtagcagcgcagcgcgaaaagctgcgagccgcactggaggacctgtacgcgacctgctgcaaccttgccagctatgtagagcagaaccggactggcttcaggaagatattgaagaagcatgacaagctggtgtcgcacccgatgtcagccatatacctgcccatcgtagaccagaagttcccggaaagccacgcagcgcacctgcgcgcacagatggacgccatcgcgtctctgtacagcatggtgtgctgcaacggcaacgcagacaaggcggcagccatcctgcgcaagcagcagcaggagcaggtgttctttgagcgcaacagcatctggaaggacatggtgggccaggagcggcgggctgccacgctgcacctgcaggatggcaaggaggctgtgcaggagtcctggctgagcacgcaccgccaggcgatgctggtcaccctcgcactggcagtgtttgccttcttactctactacccaatcttcaaggagccagagaagcagaactgcttagcgctgctggcatttgccagcatcctgtggtgcacggaggccatcccgctgtttgtgaccagcatgctggtgcccttcctcatcgtgctgctgcgggtgctggatgatgtggaccaggagccgccagctcgcctgacacctcagcaggcggcaccgcgcgtcttccacaccatgttttcgcagactatcatgctgctgcttggcggctttgccattgcagcagcgctgtctaagcactttatcgcaaagcagctggctgtggccatcctgtcgcgtgttggccgcaagccgcaccacgtgctgttggcaaacatgctcgtcgccacctttgcaagcatgtggatctcaaacgtagcagcacccgtgctctgctttagcttggtgcaacccatcttgcggaccctgcccacaaaccatgcgttctgcaaaagccttgtcctcggcatcgcacttgccagcaacctgggtggcatgacgagcccaatcagcagcccgcagaacatctttgcgattgagcgcatgagcatgggtggcagcccgcccagctggctgcagtggtttgcgatcgcgctgcctgtcagcttccttggcaatgtgctgtgctgggcggtcatcctggcggtgtacaagccagggcaaaacatcaaggaggtgcgcccgctcaagcctaatgaggaccccatgagtggcacgcaaatctacaccatcatcgtcagcttggcaactgtcacagcctggtgctgcaactcgttcctacaggcgtacactggtgagatgggtgtgctggcaatcatcccgttggtggccttttttggctttggtgtgctgtccaaggatgacttcaatggcttcctgtggaatgtggtcatgctggccatgggagggctggcgttgggggaggcagtgcagagcagtggactgctggcaaccatctcaaacttgataagcgatcttgtgggtggtcagtcgctgtgggcagtgcttgccatcttctgtgccctggtgctggtcggcacaaccttcatcagccacaccgttggggctatggtcatactgcctatcgtgcagtcagtgggagataagatgcctgggggccattccaagctgttggtgatgggagcagcactcatgtgctcaggtgctatgggcctgccagtgagtggcttcccaaacatgaacgcggtgtcgctggaggactcgaccggccagaactacatcggcacggcagacttcctcaaggtcggcgtgctgggcagcgtgctggcatacggcatcatcatcagcataggctacgggctcatgctggcggttggcttctag

SEQ ID NO: 175 - DNA, 2265 bp, Tetraselmis sp.
  source 1..2265: mol_type = other DNA; note = Tetraselmis striata
atgaagtttgagcacgcgctcgagttcaacagcgtgccggaatggcgcgggcactacctcaactacgagcagctcaagcgcctggtgtatgccgtggaggcccagcagagcgcagcgcagcgcgctagcctggacctgtcccggcggccctccggggtgcaagaggatccggaggccgggtcgccgctactgccgggcggctcggaggtggagggcggccaggaggcggaggcggagtttgtgagctgcgcggagggggagctcaagcgggtgcacgccttcctgactgcacgggaggcgggcctgctggggcagtgggaggaggcggcgcttgcggcccacagcgcggaggccagctacgtgccagcgcgcaccactcgcggaggggcgttcacgcgctcccactggtggcagcagccaacgatgcaggcgcagcggcgcacgctggtggccaccctgggcagcctctttgtgagcctgcacgacctgtccagctacgcggagctcaacgagacgggtttccgcaagatcctgaagaagcacgacaaggtgacgggcggcgcgctcaagggggcgctgctgccggtggtgcaggcccggctgggcgccaagcgcgcgcggctggatcaggcgctcgaggaggtgacgagcctctacgccacgctcgcctttgacggcgatgcggacgtcgccgcggcgcacctgagggaggggctgcgcgagcaggttgtgtttgagcgcagtgcggtgtggaaggaccgcatggaggaggagcgtcgggttgcgaccgcgcacgtcgtgggccccaaggccgccgccgccaagccgtggctgctgtccggcaaggcgattgcaggcctggcggcgctggcgctggcgggcgctgtgctgggcagcagcgcgtttggggccgacgacgctggggccaccaagcgcgcatgccttgccatcctgctggccagcgcggtgctgtggtgcaccgaggcggtgccgctctacgtgaccagcatggcgctcatctttgcggtcgtcacgctgcgcgcaatgctggacggcgacggggcgcgcctgagcgcgcccgacgccatgaagcgcgtgttctccaagatcttcagccagacggtcatgctgctgctgggcggcttcaccatggcggctgcgctctctaagcacctcatcgccaagcggctcgccattggcgtgatggcgcaggtggggcggcgcccggcctcggtgctcctggcggcgatgggcatcgcgctgttcagcagcatgtggatctccaacgtcgcggcgcccgtcctgtgcttcagcatcgtggcgcccatcctgcgcacgctgcccacggacgacccgctgggcgccgccatggtcatcggcatcgcgatggcctccaacatcggcggcatgacgtcacccatcgcgagcccgcagaacatctttgccatcgagcgcatgtccatggacggacacccgcccagctggctcgcgtggtttgcggtctccatgccggtctccatcacctgcctgctgctggtgtggcgcctcctgctcatcatctacccgatcgacagggatcaggaggtgcgcccgctgcggcagctggacgaccccttcacgctgcaccacgccttcgtcatcgcggtgtgcctggccacgatgggcctctggtgcgccaacacgtggctgctgcacctgctgggcggcatgggggtgacggcgctcatccccatggtggcgttctttggcttcgggaccctcggcaaggacgactttgagagcttcccgtggagcgtggtcatgcttgccatgggcggcatcatcctgggcgacgccgccaccgagagcgggctgctggccgccatgacagagcagattgtgggcgtcgtgggcagcctcaccgtctgcgaggtgctcgtcatcttcaccggcgtcatcgccgtcgtcaccagcttcatctcgcacaccgtgggcgccatggtcatcctgcccgtggtgcagagcatcggcgcggagctcgccaagagcaccggggtggaccacagcaagctcctggtgatgggcggagcgctgatgtgctcgggcggcatggcgctgcccgtcagcgggttccccaacatgtccgcgtcgtccatccaggaccccacgggacggaactacgtccacgtgggcgacttcctcaagaccggcatcccctccactgccatcacctggctgtgcgtcatcgccatcggctaccccatcatgtcagccatcaacctctga

SEQ ID NO: 176 - DNA, 786 bp, Trebouxia sp.
  source 1..786: mol_type = other DNA
atgaagttttcgcaggccttgaaggccaatagcgttccggactggaagcatcactacattcactactcacgcctaaagaaaatgatatttcgactggagcagctgcaaggcaacgcccctctgagtcctgtgcctgagcataggcaatccttggatttcaccaatccttcagcgcccctgctgtccagacagagctcttccatgctgcaaaggaccagttcaggccttgagcacgctcatatcgacgagctgatgtttgaacgggaaattcacgatgagctagcaagagtcaaagcattttatgttgaaaagcatgatgaactggacgcagaggtgttggcagtcttggcaaaggttgcagcagcagagagacggggcatctctggtcccggtcatcaggatgttgagggcggtcagtctttgccagaggagcagcgaatagcgttctggactgatgtgaatgtgcctaggaacatcaaggagcgcctcagtggggccctgacagacgtgtacatccagcttgacaatctatccaagtttgttgagctgaactatgatggattcaggaagatcctgaagaagcatgacaaaatgaccaacacagagctgtcagggcggctcatgcccacagtctcagacatgctggccaaggagcaacgcaaaggggctctggagggcttgaagaacagcgtggtgcatgagtacgccctcatagcacacagcggcggcgagcgtgaggccgagcaagagctggggcggcaccggcgggatcagcttgacttttga

SEQ ID NO: 177 - DNA, 33 bp, synthetic construct
  source 1..33: mol_type = other DNA
ggaattccatatgtcgctatgcaacgatctacg

SEQ ID NO: 178 - DNA, 29 bp, synthetic construct
  source 1..29: mol_type = other DNA
ccggatatcgctgccgtgaacagtacaaa

SEQ ID NO: 179 - DNA, 20 bp, synthetic construct
  source 1..20: mol_type = other DNA
agacggctgaacagtgctac

SEQ ID NO: 180 - DNA, 20 bp, synthetic construct
  source 1..20: mol_type = other DNA
cgtggagacccatatgaccg

SEQ ID NO: 181 - DNA, 18 bp, synthetic construct
  source 1..18: mol_type = other DNA
cttctcgcccatgaccac

SEQ ID NO: 182 - DNA, 20 bp, synthetic construct
  source 1..20: mol_type = other DNA
cccaccaggttgttcttcag

SEQ ID NO: 183 - DNA, 20 bp, synthetic construct
  source 1..20: mol_type = other DNA
acagcagcaacaagagcaac

SEQ ID NO: 184 - DNA, 20 bp, synthetic construct
  source 1..20: mol_type = other DNA
cgaaatcaccgaagtcaaag

(19) United States
(12) Patent Application Publication
(10) Pub. No.: US 2025/0223542 A1
(43) Pub. Date: Jul. 10, 2025

(54) MODIFIED MICROALGAE FOR ENHANCED PHOSPHATE UPTADE INVOLVING OVEREXPRESSION OF PSR1 AND OPTIONALLY UNDEREXPRESSION OF PTC1

(71) Applicant: INSTITUTE OF AGRICULTURAL RESOURCES AND REGIONAL PLANNING OF THE CHINESE ACADE, Beijing (CN)

(72) Inventors: Keke YI, Beijing (CN); Long WANG, Beijing (CN); Xianqing JIA, Beijing (CN); Lei XU, Beijing (CN)

(73) Assignee: INSTITUTE OF AGRICULTURAL RESOURCES AND REGIONAL PLANNING OF THE CHINESE ACADE, Beijing (CN)

(21) Appl. No.: 18/853,760

(22) PCT Filed: Apr. 12, 2023
(86) PCT No.: PCT/EP2023/059559

§ 371 Date: Oct. 3, 2024

(30) Foreign Application Priority Data
Apr. 14, 2022 (WO) PCT/CN2022/086874

(51) Int. Cl. C12N 1/12; C02F 3/32; C02F 101/10; C05F 11/08; C12N 15/79; C12R 1/89

(52) U.S. Cl. CPC C12N 1/12; C02F 3/322; C05F 11/08; C12N 15/79; C02F 2101/105; C12R 2001/89

(57) ABSTRACT

The invention provides a recombinant microalgal strain comprising in its genome a first modification which causes overexpression of a PSR1 gene, and optionally a further modification which reduces or eliminates expression from an endogenous PTC1 gene. The strains of the invention have utility in promoting phosphate uptake, for example from wastewater, with the microalgae then being useful as fertilisers.

### CROSS-REFERENCE TO RELATED APPLICATIONS

This patent application claims the benefit of priority of PCT/CN2022/086874 filed 14 Apr. 2022 and which is herein incorporated in its entirety.

### TECHNICAL FIELD

The present invention relates generally to recombinant microalgal strains for use in promoting phosphate uptake and their use as fertilisers.

### BACKGROUND TO THE INVENTION

As a finite, non-renewable resource, our present supply of Phosphorus (P) is primarily mined from rock P reserves and limited in a number of geographical regions (1, 2). Undue P releases increase environmental pollution due to anthropogenic activities, including industrial wastewater, municipal sewage effluent, and agricultural run-off (3). Reducing P emissions to the ecosystem is proposed as key to reducing eutrophication (4).

Therefore there is a need in the art for a system which can improve P provision for food security, while also reducing environment pollution. Sustainable and energy-efficient approaches for P recovery from waste and recycling it for agriculture are therefore required. Phosphorus is stored as inorganic phosphate (Pi) in the vacuoles of land plants but as inorganic polyphosphate (polyP) in chlorophyte algae. As an environmentally-friendly and sustainable alternative to energy-intensive and conventional biological treatment processes, enhanced biological phosphorus removal (EBPR) is increasingly employed in wastewater treatment (WWT) (5-7). EBPR systems are usually based on polyP accumulating organisms (PAO) such as bacteria and algae.

Because of their relatively low operational cost, lack of a requirement for carbon nutrition, and avoidance of sludge handling problems, algae-based EBPR systems offer competitive and attractive nutrient removal options (5). Algae can perform sustained “luxury” P uptake (i.e. take up more P than is necessary for immediate growth) driven by photosynthesis, and can grow fast while using nutrients available in wastewater. Furthermore they can form biomass suitable for bio-fertilizer production.

Recent improvements to EBPR systems include the use of membrane bioreactor (8) or optimizing processing conditions (9).

A recent study showed that loss-of-function of a tonoplast-located P transporter-Phosphate Transporter C1 (CrPTC1), containing both SPX and SLC domains (which are named after the SPX-SLC protein), caused excess polyP accumulation in acidocalcisomes and highly induced phosphorus starvation response in the model green alga Chlamydomonas reinhardtii (Chlorophyta) (13).

Patent publication CN 109970868 relates to methods for improving the content of total phosphorus and polyphosphoric acid of algae by manipulation of PTC in C. reinhardtii.

Nevertheless it can be seen that providing novel algae-based EBPR systems with improved P removal efficiency and/or maximum P accumulation capacity would provide a useful contribution to the art.

### DISCLOSURE OF THE INVENTION

The present inventors have confirmed that knock-out of the CrPTC1 gene in a C. reinhardtii, led to rapidly P removal from wastewater and high P and vacuolar polyP accumulation in cells. However the inventors then used transcriptomic analysis to show that in the Crptc1 mutant, the core regulator of P-starvation response PSR1 dependent P-starvation signaling was induced even under P sufficient conditions.

The inventors then demonstrated that PSR1 over-expression lines (PSR1-OE) showed a rapid P removal with enhanced P removal ability.

Based on their novel insights, the inventors created novel strains with high expression of PSR1 in the Crptc1 mutant background (termed herein “SPAO lines”) which unexpectedly demonstrated an excellent ability to remove and accumulate P from water without unduly compromising cell growth. P removal simulation results showed that engineered SPAO strains can remove 30 mg/L P from wastewater in 2 days, while the wild-type strains take more than 7 days.

The results disclosed herein demonstrate the utility for microalgal strains in which P-homeostasis and signaling are simultaneously modified in order to enhance the efficiency of P removal from the environment. In preferred embodiments P vacuolar transport is also modified.

Some of the findings of the present application were published after the presently claimed priority date (Wang, L., Jia, X., Xu, L., Yu, J., Ren, S., Yang, Y., Wang, K., López-Arredondo, D., Herrera-Estrella, L., Lambers, H. and Yi, K. (2023), Engineering microalgae for water phosphorus recovery to close the phosphorus cycle. Plant Biotechnol J. https://doi.org/10.1111/pbi.14040).

Thus in one aspect there is provided a recombinant microalgal strain comprising in its genome a modification which causes overexpression of a PSR1 gene.

By “recombinant microalgae” is meant a microalgae in which a nucleic acid sequence contains at least one targeted genetic alteration introduced by man that distinguishes the engineered cell from the naturally occurring cell. Such microalgae may also be referred to as “engineered” or “modified”. Thus the microalgal strains of the invention are non-naturally occurring, owing to their genetic modifications. Recombinant microalgae can be prepared by transformation or other known molecular biology techniques as further detailed below.

The term “overexpression” as used herein refers to excessive expression of a gene product (RNA or protein, here for PSR1) in greater-than-normal amounts (i.e. compared to the same strain lacking the modification). Therefore this encompasses the introduction of a PSR1 transgene, leading to greater amounts of PSR1 polypeptide than would otherwise have been the case.

Overexpression of a PSR1 gene modulates P homeostasis or signalling, and in particular triggers starvation signalling, so as to promote P uptake compared to a parent strain lacking said modification. More specifically, and without being bound by mechanism, it is believed that over-expression of PSR1 promotes Pi acquisition through directly up-regulating the expression of P starvation-induced genes (PSIGs) which are responsible for Pi absorption from the extracellular environment and alkaline phosphatases (ALPs) which liberate soluble reactive phosphorus from dissolved organic P compounds.

Microalgae encompass a broad range of organisms, mostly unicellular aquatic organisms. The unicellular eukaryotic microalgae (including green algae, diatoms, and brown algae) are photosynthetic and have a nucleus, mitochondria and chloroplasts.

Preferably the microalgae are fresh water algae.

Preferably the microalgae are Chlorophyta (unicellular green algae), more preferably said microalgae is chosen from the group consisting of Chlamydomonas, Chlorella, and Scenedesmaceae

Even more particularly said microalgae is chosen from the group consisting of Chlamydomonas, more particularly Chlamydomonas reinhardtii. C. reinhardtii is a eukaryote distributed in various environments such as fresh water and oceans. An example strain is C. reinhardtii strain CC-4533.

In one embodiment the microalgae is selected from the following species: Asteromonas gracilis, Botryococcus terribilis, Carteria crucifera, Chlamydomonas bilatus, Chlamydomonas eustigma, Chlamydomonas incerta, Chlamydomonas noctigama, Chlamydomonas schloesseri, Chlamydomonas sp.-M2762, Chromochloris zofingiensis, Coccomyxa subellipsoidea C-169, Cylindrocapsa geminella, Edaphochlamys debaryana, Enallax costatus, Entransia fimbriata, Eudorina elegans, Golenkinia longispicula, Gonium pectorale, Haematococcus pluvialis, Hafniomonas reticulata, Ignatius tetrasporus, Mesostigma viride, Monoraphidium neglectum, Oedogonium cardiacum, Oedogonium foveolatum, Pandorina morum, Phacotus lenticularis, Planophila terrestris, Pteromonas angulosa, Raphidocelis subcapitata, Scherffelia dubia, Stephanosphaera pluvialis, Symbiochloris reticulata, Tetradesmus deserticola, Tetraselmis chui, Tetraselmis striata, Trebouxia sp. A1-2, Vitreochlamys sp, Volvox aureus-M1028, Volvox aureus-M2242, Volvox globator,

Whole genome sequencing information is available for all of these strains, and PSR and PTC polypeptide and nucleic acid sequences are provided herein (SEQ ID Nos 1 to 176), as shown in Table 1.

In the light of the present disclosure those skilled in the art can readily provide mutants according to the present invention in these species. It will be appreciated that where the invention is discussed in relation to C. reinhardtii, unless context demands otherwise, that discussion will apply mutatis mutandis to these other strains.

In C. reinhardtii, it is known that P deficiency response is regulated by the MYB-CC gene-Phosphate Starvation-Responsive 1 (PSR1) (10, 11). PSR1 is believed to be a global transcriptional regulator of phosphorus deficiency responses and carbon storage metabolism (12).

In one embodiment the overexpressed PSR1 gene is the PSR1 from a species shown in Table 1 hereinafter.

In one embodiment the overexpressed PSR1 gene has the sequence of any of SEQ ID No 2, or any of SEQ ID Nos 48 to 70, or 72 to 90 or is a homologue or derivative or genomic equivalent thereof.

For example the gene may encodes a PSR1 polypeptide having at least 75, 80, 85, 90, 95, 96, 97, 98, 99% identity with any of SEQ ID No 1, or any of SEQ ID Nos 5 to 27, or 29 to 47.

For example the gene may encode a homologue of a PSR1 polypeptide, for example as shown in SEQ ID No 71 (which is a homologue of SEQ ID No 70). That encodes a polypeptide having SEQ ID No 28.

In one embodiment the overexpressed PSR1 gene is the PSR1 from C. reinhardtii gene or a homologue or derivative thereof.

In one embodiment the overexpressed PSR1 gene has SEQ ID 2 or is a homologue or derivative thereof.

For example the gene may encode a PSR1 polypeptide having at least 75, 80, 85, 90, 95, 96, 97, 98, 99% identity with SEQ ID 1.

Homologues and derivatives (collectively “variants”) are discussed in more detail hereinafter.

In one embodiment overexpression is achieved by up-regulation of an endogenous PSR1 gene.

For example the strain and respective PSR1 gene may be selected from those described in Table 1.

In another embodiment overexpression is achieved by expression of a PSR1 transgene.

Such a PSR1 transgene may be same as an endogenous gene in the strain, or may be heterologous to the strain.

Methods for up-regulation of endogenous genes, and expression of transgenes, are discussed in more detail hereinafter.

In a preferred embodiment, in addition to the first modification relating to PSR1 gene expression, the recombinant microalgal strain comprises in its genome a further (second) modification which reduces or eliminates expression from an endogenous gene (thereby reducing production of an endogenous PTC1 polypeptide).

The PTC1 polypeptide is a tonoplast-located Pi efflux transporter. It comprises both SPX and SLC domains (13).

Therefore this (second) modification is a loss of function modification which inhibits the tonoplast-located P transporter, thereby inhibiting vacuolar P export transport and thereby increasing accumulation of inorganic polyphosphate (polyP) in vacuoles compared to a parent strain lacking said modification.

In one embodiment the strain is of a species shown in Table 1 and/or the PTC1 gene is a gene identified therein, or is a homologue thereof.

In one embodiment the gene or sequence encoding the endogenous PTC1 polypeptide comprises the sequence as shown in SEQ ID 4, or any of SEQ ID Nos 134 to 166 or 168 to 176, or is a homologue of any of those.

For example the gene may encode a homologue of a PTC1, for example as shown in SEQ ID No 167 (which is a homologue of SEQ ID No 166). That encodes a polypeptide having SEQ ID No 124.

The endogenous PTC1 protein may have any of the sequences shown in SEQ ID No 3, or any of SEQ ID Nos 91 to 123 or 125 to 133 or is a homologue thereof.

The PTC1 protein may have the sequence shown in SEQ ID NO: 3.

In one embodiment the gene or sequence encoding the endogenous PTC1 polypeptide has SEQ ID 3 or is a homologue thereof.

In another embodiment the gene is a native gene to the microalgal strain that is homologous to the Chlamydomonas reinhardtii PTC1 gene, for example the homologous PTC1 gene it has greater than least 55, 60, 65, 70, 75, 80, 85, 90, 95, 96, 97, 98, 99% homology to the CDS of said gene.

The encoded endogenous PTC1 polypeptide may share at least 75, 80, 85, 90, 95, 96, 97, 98, 99% identity with SEQ ID 3

As explained above the second modification down-regulates or inactivates the PTC1 gene (e.g. knocks it out, or down).

Such a modification can be achieved using a number of methods known in the art. For example utilising chemical mutagenesis and selection, genome editing, or an inducible promoter and trans acting elements. Gene silencing (for example based on RNA technologies) may also be used.

In one embodiment the gene is rendered non-functional. For example the endogenous gene may include an insertion within it which renders it non-functional, or the gene may be substantially deleted.

Methods for down regulation or inactivation of an endogenous gene are discussed in more detail hereinafter.

In one embodiment of the invention, the strain of the invention is in the form of biologically pure culture of said strain (isolated from any contaminants), which may be a slope culture or liquid medium broth. In another embodiment it is in the form of a freeze dried sample, a liquid nitrogen frozen sample, or a frozen preparation in glycerol of said strain.

In another aspect there is provided a cell extract; a cell suspension; a cell homogenate; a cell lysate; or a cell pellet of a strain of the invention.

In another aspect there is provided a culture broth of said strain, which may be cell free or substantially cell free.

In another aspect there is provided a process for producing a recombinant microalgal strain as described above, having enhanced P removal efficiency (PRE), the method comprising introducing the first genetic modification described above into a parent strain such as to cause overexpression of the PSR1 gene.

In one embodiment the process further comprises (in any order) introducing the second genetic modification described above into a parent strain such as to eliminate or reduce expression of an endogenous PTC1 polypeptide.

Alternatively the second genetic modification may be pre-existing in a modified parent strain, and the first genetic modification described above is introduced into the modified parent strain such as to cause overexpression of the PSR1 gene.

The processes may be used, inter alia, to achieve one or more of the following:
(1) increasing the capacity for polyphosphoric acid content of the resulting microalgae;
(2) increasing the capacity for total phosphorus content of the microalgae;
(3) increasing alkaline phosphatase activity of microalgae;

In another aspect there is provided a recombinant microalgal strain obtained or obtainable by these processes. For example a recombinant microalgal strain obtained by introducing and expressing a PSR1 gene into a recipient microalgae in which the endogenous PTC1 gene has been impaired as described herein.

In another aspect there is provided a recombinant microalgal strain as described herein capable of accumulating (e.g. from P-containing wastewater) a total P concentration of at least 30, 40, 50, 60 mg g−1 DW e.g. up to 70 mg g⁻¹ DW e.g. about 68 mg g⁻¹ DW.

In another aspect there is provided a recombinant microalgal strain as described herein having a total P concentration of at least 30, 40, 50, 60 mg g⁻¹ DW e.g. up to 70 mg g⁻¹ DW e.g. about 68 mg g⁻¹ DW

In another aspect there is provided a recombinant microalgal strain as described herein capable of accumulating (e.g. from P-containing wastewater) a total P concentration of at least 3%, 4%, 5%, 6% e.g. up to 7%.

In another aspect there is provided a recombinant microalgal strain as described herein having a total P concentration of at least 3%, 4%, 5%, 6% e.g. up to 7%.

As demonstrated in the Examples hereinafter, the first and second modifications described above lead to increased ‘luxury’ P uptake, and increase total P and polyP in the recombinant strain, thereby improving its overall PRE.

For example, based on an initial P content of 30 mg/L and an initial inoculation amount of alga of 10⁵ cells, preferred strains according to the invention can remove all P in the medium after 60 hours, as compared to a wild-type strain requiring 9 days, as shown in the follow table.

| | WT | | SPAO24 | |
|---|---|---|---|---|
| | P in medium | P removal | P in medium | P removal |
| Hours | (mg/L) | rate | (mg/L) | rate |
| 0 | 30.79 | 100.0% | 30.36 | 100.0% |
| 12 | 30.82 | 100.1% | 31.30 | 103.1% |
| 24 | 29.84 | 96.9% | 27.59 | 90.9% |
| 36 | 28.94 | 94.0% | 20.42 | 67.3% |
| 48 | 24.25 | 78.8% | 6.90 | 22.7% |
| 60 | 20.50 | 66.6% | 0 | 0.0% |
| 72 | 16.04 | 52.1% | 0 | 0.0% |
| 96 | 12.02 | 39.1% | 0 | 0.0% |
| 120 | 9.48 | 30.8% | 0 | 0.0% |
| 144 | 5.28 | 17.1% | 0 | 0.0% |
| 168 | 1.21 | 3.9% | 0 | 0.0% |
| 192 | 0.77 | 2.5% | 0 | 0.0% |
| 216 | 0.01 | 0.0% | 0 | 0.0% |
| 240 | 0 | 0.0% | 0 | 0.0% |

In one embodiment the strains of the invention demonstrate at least a 10, 20, 30, 40, 50, 60, 70, 80, 90, 100, 200% increase in any of total P or polyP in the strain after culture for 60 hours under comparable conditions compared to a parent strain (for example a wild-type strain lacking said modification or modifications, or a parent strain including only the 2nd modification.

In one embodiment the strains of the invention demonstrate at least a 10, 20, 30, 40, 50, 60, 70, 80, 90, 100, 200% increase in PRE by the strain after culture for 60 hours under comparable conditions compared to a parent strain (for example a wild-type strain lacking said modification or modifications, or a parent strain including only the 2nd modification.

In one embodiment the strains of the invention demonstrate at least a 10, 20, 30, 40, 50, 60, 70, 80, 90, 100, 200% decrease in complete-removal time of total P in a medium in which the strain is cultured compared to a parent strain cultured under comparable conditions (for example a wild-type strain lacking said modification or modifications, or a parent strain including only the 2nd modification.

In one embodiment the strains of the invention demonstrate at least a 10, 20, 30, 40, 50, 60, 70, 80, 90, 100, 200% decrease in total P amount in the medium in which the strain is cultured for 60 hours compared to a parent strain cultured under comparable conditions (for example a wild-type strain lacking said modification or modifications, or a parent strain including only the 2nd modification.

Some of these aspects and embodiments will now be discussed in more detail.

It will be recognised that whenever a particular protein or nucleic acid is referred to herein e.g. with reference to an accession number or SEQ ID NO., the invention applies mutatis mutandis to variants (e.g. homologues or derivatives).

For example where PSR1 gene from C. reinhardtii is discussed, a homologue or derivative thereof may be used to achieve overexpression. Such a homologue or derivative will encode a polypeptide sharing the biological activity of the C. reinhardtii PSR1 i.e. MYB-CC polypeptide which shares sequence identity with that PSR1 as well as the ability to regulate the P deficiency response. As explained above, and without being bound by mechanism, it is believed that PSR1 promotes Pi acquisition through directly up-regulating the expression of P starvation-induced genes (PSIGs) and alkaline phosphatases (ALPs).

For example where PTC1 gene from Chlamydomonas reinhardtii is discussed, a homologue thereof may be targeted to reduce or eliminate its expression in the respective host microalga. Such a homologue will encode a polypeptide which shares sequence identity with that PTC1 as well as sharing the biological activity of the C. reinhardtii PTC1 i.e. a tonoplast-located P transporter which catalyses vacuolar P export.

The term “identity” refers to sequence similarity to a reference sequence. Identity can be evaluated using the naked eye or computer software. Using computer software, the identity between two or more sequences can be expressed in percentage (%), which can be used to evaluate the identity between related sequences.

Sequence identity may be assessed as using BLASTp (proteins) or Megablast (nucleic acids) from NCBI (http://www.ncbi.nlm.nih.gov/blast/Blast.cgi) using default settings.

Variants of the sequences disclosed herein (for example any of those shown in Table 1) preferably share at least 55%, 56%, 57%, 58%, 59%, 60%, 65%, or 70%, or 80% identity, most preferably at least about 90%, 95%, 96%, 97%, 98% or 99% identity. Such variants may be referred to herein as “substantially homologous”.

In specific embodiments, two nucleic acid sequences are “substantially homologous” when at least about 55% or at least about 99% of the nucleotides (or any integer value in between) match over a defined length of the nucleic acid sequences i.e. they share this level of identity as determined by a sequence comparison algorithm such as BLAST.

Substantially homologous nucleic acids may be those which hybridize (to the respective complement of) a nucleotide sequence described herein e.g. encoding the PSR1 or PTC1 sequences of Chlamydomonas reinhardtii under stringent conditions e.g. hybridization in a solution of 2×SSC, 0.1% SDS at 68° C. for 2 times, 5 min each time, and in a solution of 0.5×SSC, 0.1% SDS, at 68° C. (washing the membrane 2 times, each time 15 min).

Similarly, in particular embodiments of the invention, two amino acid sequences are “substantially homologous” when greater than 75% of the amino acid residues are identical wherein identical contemplates a conservative substitution at a nucleic acid position. In a preferred embodiment at least 99% of the amino acid residues are identical (or any integer value in between).

The term “homologous” or “homologues” refers to the relationship between two genes or proteins that possess a “common evolutionary origin”, and embraces alleles (which will include polymorphisms or mutations at one or more bases), paralogues, isogenes, or other homologous genes belonging to the same families as the relevant enzymes.

Also included are orthologues or homologues from different microbial or other species. The invention embraces upregulation of a PSR1 sequence in the strain (either native or transgenic) which is substantially homologous to the PSR1 sequences of C. reinhardtii.

The invention embraces reducing or eliminating expression of an endogenous PTC1 sequence in the strain which is substantially homologous to the PTC1 sequences of C. reinhardtii.

“Derivatives” (in relation to the PSR1 transgenes used in the invention, or their encoded polypeptides) may be prepared, for instance, by site directed or random mutagenesis, or by direct synthesis. Preferably the variant nucleic acid is generated either directly or indirectly (e.g. via one or more amplification or replication steps) from an original nucleic acid having all or part of a sequence referred to herein.

Changes (“mutations”) may be desirable for a number of reasons. For instance they may introduce or remove restriction endonuclease sites or alter codon usage.

Alternatively changes to a sequence may produce a derivative by way of one or more (e.g. several) of addition, insertion, deletion or substitution of one or more nucleotides in the nucleic acid, leading to the addition, insertion, deletion or substitution of one or more (e.g. several) amino acids in the encoded polypeptide.

Other desirable mutations may be random or site directed mutagenesis in order to alter or evolve the activity (e.g. specificity) or stability of the encoded polypeptide. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation. Also included are variants having non-conservative substitutions. As is well known to those skilled in the art, substitutions to regions of a peptide which are not critical in determining its conformation may not greatly affect its activity because they do not greatly alter the peptide's three dimensional structure. In regions which are critical in determining the peptides conformation or activity such changes may confer advantageous properties on the polypeptide. Indeed, changes such as those described above may confer slightly advantageous properties on the peptide e.g. altered stability or specificity.

Derivatives include of fragments of the full-length polypeptides disclosed herein, especially active portions thereof. An “active portion” of a polypeptide means a peptide which is less than said full length polypeptide, but which retains its essential biological activity.

Also included are nucleic acids corresponding to those above, but which have been extended at the 3′ or 5′ terminus.

The term ‘variant’ nucleic acid as used herein encompasses all of these possibilities. When used in the context of polypeptides or proteins it indicates the encoded expression product of the variant nucleic acid.

As explained above, overexpression of PSR1 is typically achieved by introduction of a transgene encoding a PSR1, or by enhancement of expression of native PSR1 gene.

Generally speaking, those skilled in the art are well able to construct vectors and design protocols for recombinant gene expression (e.g. for expressing a heterologous nucleic acid within a host or one or more cells of a host). Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

“Expression vector” or “transformation vector” or “recombinant DNA construct”, or similar terms, are defined herein as DNA sequences that are required for the transcription of recombinant genes and the translation of their mRNAs in the microalgae algae host cells.

“Expression vectors” contain one or more expression cassettes for the recombinant genes (one or more gene encoding the protein, peptide or polypeptide of interest and often selectable markers). A vector including nucleic acid according to the present invention need not include a promoter or other regulatory sequence, particularly if the vector is to be used to introduce the nucleic acid into cells for recombination into the genome. In the case of chloroplast genome transformation, expression vectors will typically contain homologous recombination regions for the integration of expression cassettes inside the chloroplast genome.

Preferably the nucleic acid in the vector is under the control of, and operably linked to, an appropriate promoter or other regulatory elements for transcription in the host algal cell.

For microalgae chloroplast expression, promoters, 5′UTRs and 3′UTRs that can be used in the context of the invention are for example: the promoters and 5′UTRs of the genes psbD, psbA, psaA, atpA, and atpB, the 16S rRNA promoter (Prrn) promoter fused with a 5′UTR, the psbA 3′ UTR, the atpA 3′UTR or the rbcL 3′ UTR.

A 5′UTR from exogenous origin as for instance the 5′UTR of the gene 10L of the bacteriophage T7 can be used also fused downstream a microalgae promoter. In particular, the nucleic acid sequence is operationally linked at its 5′end to the C. reinhardtii 16S rRNA promoter (Prrn).

Stable expression and translation of the nucleic acid sequence according to the present invention can for example be controlled by the promoter and 5′UTR from psbD and the atpA 3′UTR.

US2012/0208201 describes methods of enhanced gene expression algae, using an algae compatible transcriptional promoter functionally upstream of a coding sequence for a gene expression enhancer (GEE) fusion protein.

Vectors for use in the invention may comprise a plasmid capable of integrating the DNA sequence of interest into a chromosome of the algae. There are a large numbers of such vectors known and characterized. A preferred vector of the invention is pSP124 (Lumbreras et al., Efficient foreign gene expression in C. reinhardtii mediated by an endogenous intron, The Plant Journal 14 (4): 441-447 (1998)).

Embodiments of the present invention may use one or more vectors to introduce a cassette encoding PSR1 and a gene silencing inhibitor into the nucleus DNA of algae. A gene silencing inhibitor is a peptide that induces relaxation of nucleosomes in the algae's nucleus. Gene silencing inhibitors include histone acetyl transferases (HATs) and other peptides that modify elements of the nucleosome, causing the chromatin structure to relax and to allow transcription factors to access the gene of interest. HAT proteins and the HAT domains of p300 and of other HAT proteins are known to cause histone acetylation and can be utilized in the invention. In accordance to the invention the domain responsible for the acetylation activity or the whole protein is deployed. See Fukuda H, et al., Brief Funct. Genomic Proteomic, 5 (3): 190-208 (2006); Renthal W. and Nestler E. J., Semin Cell Dev Biol. 20 (4): 387-94 (Epub 2009); and Lin Y. Y. et al., Genes Dev., 22 (15): 2062-74 (2008).

As explained in WO2021/170754, the chloroplast genome of microalgae host cell can be targeted for transformation according to any suitable techniques well known by the man skilled in the art including, without limitations biolistics (Boynton et ai, 1988; Goldschmidt-Clermont, 1991), electroporation (Fromm et ai, Proc. Natl. Acad. Sci. (USA) (1985) 82:5824-5828; see Maruyama et at. (2004), Biotechnology Techniques 8:821-826), glass bead transformation, protoplasts treated with CaCh and polyethylene glycol (PEG) (see Kim et ai (2002), Mar. Biotechnol. 4:63-73) or microinjection.

WO2014/076571 describes a variety of different methods for transfecting vectors into algal cells nuclei or chloroplasts. In various embodiments, vectors can be introduced into algae nuclei by, for example without limitation, electroporation, magnetophoresis. The latter is reportedly a nucleic acid introduction technology using the processes of magnetophoresis and nanotechnology fabrication of micro-sized linear magnets (Kuehnle et al., U.S. Pat. No. 6,706,394; 2004; Kuehnle et al., U.S. Pat. No. 5,516,670; 1996) that proved amenable to effective chloroplast engineering in freshwater Chlamydomonas (Champagne et al., Magnetophoresis for pathway engineering in green cells. Metabolic engineering V: Genome to Product, Engineering Conferences International Lake Tahoe CA, Abstracts pp 76; 2004).

To identify the microalgal transformants, a selectable marker gene may be used. Mention may be made for example of the aadA gene coding aminoglycoside 3″-adenylyltransferase and conferring the resistance to spectinomycin and streptomycin in the case of C. reinhardtii chloroplast transformation.

Transformed algae can be recovered on a solid nutrient media or in liquid media. Elizabeth H Harris, Chlamydomonas As A Model Organism, Annual Review of Plant Physiology and Plant Molecular Biology 52:363-406 (2001) and EMBO Practical Course: Molecular Genetics of Chlamydomonas, Laboratory protocols. Geneva, Sep. 18-28, 2006.

As explained above, reduction or elimination of expression of an endogenous PTC1 polypeptide can be achieved in a variety of ways. For example direct gene knockout or knockdown (e.g. by modification of the encoding gene acting in cis), or gene silencing acting in trans.

Such a modification can be achieved using a number of methods known in the art. For example utilising chemical mutagenesis and selection, genome editing, or an inducible promoter and trans acting elements. Gene silencing (for example based on RNA technologies) may also be used.

In one embodiment the gene is rendered non-functional. For example the endogenous gene may include an insertion within it which renders it non-functional, or the gene may be substantially deleted.

The term “knockout” or “gene knockout” refers herein to any organism and/or its corresponding genome where the gene of interest has been rendered unable to perform its function. This can be accomplished by both classical mutagenesis, natural mutation, specific or random inactivation, targeting in cis or trans, or any method wherein the normal expression of a protein is altered to reduce its effect. For example but not to limit the definition:
1) one can use chemical mutagenesis to damage the gene and then select for organisms not expressing the gene,
2) one can target the gene and remove a portion or all of the gene by homologous recombination,
3) one can use RNAi methods to produce an inhibitor molecule for a particular protein and similar methods and
4) one can use genome editing tools (i.e. CRISPR-Cas) to specifically modify the gene.

For example, to permanently inactivate PTC1 a plasmid can be constructed for gene deletion by integrational mutagenesis or gene replacement techniques well known in the art. Integrational mutagenesis and gene replacement can selectively inactivate undesired genes from host genomes. In this technique, a fragment of the target gene is cloned into a non-replicative vector with a selection marker, resulting in the non-replicative integrational plasmid. The partial gene in the non-replicative plasmid can recombine with the internal homologous region of the original target gene in the parental chromosome (double crossover), which results in the insertional inactivation of the target gene. The use of gene replacement (by double recombination) may be preferred to insertional inactivation (single recombination) since it permits the generation of more stable engineered strains, without the need to maintain selection of vectors.

Down regulation may be achieved by methods known in the art, for example using anti-sense technology.

In using anti-sense genes or partial gene sequences to down-regulate gene expression, a nucleotide sequence is placed under the control of a promoter in a “reverse orientation” such that transcription yields RNA which is complementary to normal mRNA transcribed from the “sense” strand of the target gene. See, for example, Rothstein et al, 1987; Smith et al, (1988) Nature 334, 724-726; Zhang et al, (1992) The Plant Cell 4, 1575-1588, English et al., (1996) The Plant Cell 8, 179-188. Antisense technology is also reviewed in Bourque, (1995), Plant Science 105, 125-149, and Flavell, (1994) PNAS USA 91, 3490-3496.

An alternative to anti-sense is to use a copy of all or part of the target gene inserted in sense, that is the same, orientation as the target gene, to achieve reduction in expression of the target gene by co-suppression. See, for example, van der Krol et al., (1990) The Plant Cell 2, 291-299; Napoli et al., (1990) The Plant Cell 2, 279-289; Zhang et al., (1992) The Plant Cell 4, 1575-1588, and U.S. Pat. No. 5,231,020. Further refinements of the gene silencing or co-suppression technology may be found in WO95/34668 (Biosource); Angell & Baulcombe (1997) The EMBO Journal 16,12:3675-3684; and Voinnet & Baulcombe (1997) Nature 389: pg. 553.

Double stranded RNA (dsRNA) has been found to be even more effective in gene silencing than both sense or antisense strands alone (Fire A. et al Nature, Vol 391, (1998)). dsRNA mediated silencing is gene specific and is often termed RNA interference (RNAi) (See also Fire (1999) Trends Genet. 15:358-363, Sharp (2001) Genes Dev. 15:485-490, Hammond et al. (2001) Nature Rev. Genes 2:1110-1119 and Tuschl (2001) Chem. Biochem. 2:239-245).

RNA interference is a two-step process. First, dsRNA is cleaved within the cell to yield short interfering RNAs (siRNAs) of about 21-23nt length with 5′ terminal phosphate and 3′ short overhangs (˜2nt) The siRNAs target the corresponding mRNA sequence specifically for destruction (Zamore P. D. Nature Structural Biology, 8, 9, 746-750, (2001)

Another methodology known in the art for down-regulation of target sequences is the use of “microRNA” (miRNA) e.g. as described by Schwab et al 2006, Plant Cell 18, 1121-1133. This technology employs artificial miRNAs, which may be encoded by stem loop precursors incorporating suitable oligonucleotide sequences, which sequences can be generated using well defined rules in the light of the disclosure herein.

Thus in various embodiments the invention may provide methods for influencing or affecting PRE in an algal host which method comprises any one or more of: (i) causing or allowing transcription from a nucleic acid encoding a PSR1 polypeptide (which may be a native one or active variant thereof, or heterologous to the host); (ii) causing or allowing transcription from a nucleic acid (a) comprising the complement sequence of a PTC1 nucleotide sequence such as to reduce the respective encoded polypeptide activity by an antisense mechanism; (b) encoding a stem loop precursor comprising 20-25 nucleotides, optionally including one or more mismatches, of PTC1 nucleotide sequence such as to reduce the respective encoded polypeptide activity by an miRNA mechanism; (c) encoding double stranded RNA corresponding to 20-25 nucleotides, optionally including one or more mismatches, of a PTC1 nucleotide sequence such as to reduce the respective encoded polypeptide activity by an siRNA mechanism.

WO2014/076571 describes methods of modifying algae genomes, based on the use of rare-cutting endonuclease, especially a homing endonuclease or a TALE-Nuclease, being expressed over several generations to efficiently modify said target sequence

WO2019/200318 gives examples of systems for genetically modifying algal genomes, such as a CRISPR/Cas system (e.g., a type I, II, or III CRISPR/Cas system, as well as modified versions thereof, such as a CRISPR/dCas9 system), TALENs, or zinc fingers to accomplish the desired genomic editing.

US2019/0045812 describes mutants constructed by using CRISPR gene scissors technology (RGEN RNPs) without any introduction of an exogenous DNA in a microalga C. reinhardtii to knock out a target gene.

US2018/0187170 describes Chlamydomonas reinhardtii knockout lines generated in different parental backgrounds.

In another aspect there are provided uses of a recombinant microalgal strain of the invention to reduce Pi or organophosphorus in an environment (e.g. external environment) in which said strain is present or introduced.

Strains of the invention may optionally be used in mixed consortia to maximise effectiveness and versatility, including mixed microalgae-bacteria consortia.

Thus there is a provided a method of reducing Pi or organophosphorus in an environment, the method comprising introducing or culturing strain of the invention in the environment. Methods of culturing the strains are described hereinafter.

Typically the environment is an aqueous environment e.g. a water body, which is optionally is or comprises waste water from a municipal or agricultural source (e.g. aquaculture pond, or agricultural flow-off). For example the microalgae may be used to treat Primary settled wastewater (PSW) or secondary treatment effluent (STE). However the strains may be used in other aqueous environments, or even terrestrial ones where there is sufficient water present e.g. through flooding or waterlogging.

The methods of the invention may comprise a batch process by which the strains are added to the environment, and optionally removed at intervals for utility as a fertiliser (see below).

Alternatively the methods may comprise continuous flow processes, by which the strains are immobilised or suspended and exposed continuously to a water stream or flow from which Pi or organophosphorus is to extracted, and optionally removed at intervals for utility as a fertiliser (see below).

General systems for continuous flow microalgal cultures are known in the art, for example by using algae-anchored fiber spheres, or other established technologies such as Membrane Bioreactors (MBRs) (Chen et al., 2018; Qin et al., 2020), or Sequencing Batch Reactors (SBRs) (Acevedo et al., 2012). A further publication (P. D. Álvarez-Díaz, J. Ruiz, Z. Arbib, J. Barragán, M. C. Garrido-Pérez, J. A. Perales. Examples of MBRs are shown in FIG. 9.

As explained in the introduction, enhanced biological phosphorus removal (EBPR) is already employed in wastewater treatment (5). An example microalgae-based wastewater treatment (MBWT) process is shown in FIG. 6, and example designs are shown in FIG. 7 (both discussed more fully in Reference 5). Commonly used designs include open raceway ponds (RPs), tubular photobioreactors (PBRs), flat panel (FP) PBRs, soft frame PBRs and other hybrid PBRs. PBRs can be based on vertical tubes.

Any of these systems may be utilised with the modified strains of the present invention. In these systems the strains may optionally be suspended or immobilised.

Mohsenpour, Seyedeh Fatemeh, et al. “Integrating micro-algae into wastewater treatment: A review.” Science of the Total Environment 752 (2021): 142168, describes in detail mechanisms for P removal by microalgae, abiotic and biotic factors influencing micro-algae wastewater treatment, and microalgae bioreactor configurations for wastewater treatment.

WO2017/165290 describes methods and apparatus for cultivating algae biomass in which auto-flocculating (self-aggregated) species of algae that are grown in raceways under controlled culture conditions such as controlled water velocity and controlled composition of the algae growth medium. The apparatus for growing algae biomass (referred to therein as a “Sustainable Algae Floe with Recirculation” (“SAFR”) apparatus”) comprises:
at least one Algae Growth Raceway (AGR);
an Algae Growth Medium (AGM) reservoir functionally connected to the AGR,
at least one AGM flow disrupter positioned in the AGR; and
an AGM circulation system (e.g., pump) for circulating AGM through the at least one AGR.

The SAFR apparatus, systems, and methods are reported to find applications in water treatment, such as removal of nutrients (e.g. phosphorus) from waste water, eutrophic aquifers and aquaculture.

Culture systems may be based on the use of in situ treatment of aqueous environments e.g. aquaculture systems. Culture systems suitable for this purpose include permeable floating photobioreactors. Culture systems may be based around autotrophic or split-mixotrophic systems, in which additional organic carbon is supplied e.g. during hours of darkness.

These and other types of system for wastewater treatment are generally known in the art, and discussed (for example) in the following: Wollmann, F., Dietze, S., Ackermann, J.-U., Bley, T., Walther, T., Steingroewer, J. and Krujatz, F. (2019) Microalgae wastewater treatment: Biological and technological approaches. Engineering in Life Sciences, 19, 860-871.

Microalgal biofilms and their use in the treatment of wastewaters are described by Miranda, A. F., Ramkumar, N., Andriotis, C., et al. (2017) Applications of microalgal biofilms for wastewater treatment and bioenergy production. Biotechnology for Biofuels, 10, 120. Algal biofilm reactors are discussed by Choudhary, P., Prajapati, S. K., Kumar, P., Malik, A. and Pant, K. K. (2017) Development and performance evaluation of an algal biofilm reactor for treatment of multiple wastewaters and characterization of biomass for diverse applications. Bioresource Technology, 224, 276-284—see FIG. 8 herein.

The publication “Freshwater microalgae selection for simultaneous wastewater nutrient removal and lipid production”, Algal Research, Volume 24, Part B, 2017, Pages 477-485) describes how microalgae may be used as both bioenergy sources as well as wastewater pollution reducers.

Solovchenko, A., Verschoor, A. M., Jablonowski, N. D. and Nedbal, L. (2016) “Phosphorus from wastewater to crops: An alternative path involving microalgae”. Biotechnology Advances, 34, 550-564, describes how the ability of microalgae to accumulate large quantities of P can be a way to direct waste P back to crop plants. As noted therein, algae can acquire and store P through luxury uptake, and the P enriched algal biomass can be used as bio-fertilizer. In particular, cultivation technologies can be used for solar-driven recycling of P and other nutrients from wastewater into algae-based bio-fertilizers.

Such systems can be used analogously with the strains of the present invention.

In one embodiment the uses or methods described above comprise the further step of recovering the strain following a period of culture in the environment and utilising the same as a P-rich fertiliser.

It has been demonstrated that the P in microalgae can be rapidly transformed in soil and mobilized for plant growth (Siebers et al., 2019). Optionally the strains of the invention, having accumulated luxury P, can be combined with a further microorganism which enhances degradation of polyp to inorganic P.

The microalgae strains of the present invention may be used in slow-release or liquid bio-fertilisers. Typically the production process of slow-release algal fertilizer involves the algae cultivation, biomass dehydration, and biomass pasteurization or pulverization (see e.g. Zou, Y., Zeng, Q., Li, H., Liu, H. and Lu, Q. (2021) “Emerging technologies of algae-based wastewater remediation for bio-fertilizer production: a promising pathway to sustainable agriculture”. Journal of Chemical Technology & Biotechnology, 96, 551-563).

Microalgae may be utilised as a hydrochar. An example processes for production utilises harvested biomass and a reactor heated to 200-300 C at 3 C/min, and held at the final temperature for a duration of 2 h. The reactor is then rapidly cooled down to room temperature using a recirculating condensing engine. The solid and liquid products are separated by centrifugation and fully gravity-filtered through a 0.45 mm membrane (see e.g. Chu, Q., Lyu, T., Xue, L., et al. (2021) Hydrothermal carbonization of microalgae for phosphorus recycling from wastewater to crop-soil systems as slow-release fertilizers. Journal of Cleaner Production, 283, 124627).

In a further aspect the invention provides a fertiliser product obtained from the methods described above e.g. comprising, consisting or consisting essentially of a strain of the invention (once it has been cultured in the P containing environment, and having accumulated luxury P).

Optionally this comprises further biological or chemical components e.g. further microorganisms.

The effectiveness of algae based fertilisers has been demonstrated in the study Mulbry, W., Kondrad, S., Pizarro, C., Kebede-Westhead, E., 2008. Treatment of dairy manure effluent using freshwater algae: algal productivity and recovery of manure nutrients using pilot-scale algal turf scrubbers. Bioresour. Technol. 99, 8137-8142. The authors demonstrated that 20-day-old cucumber and corn seedlings grown in a potting mix-containing algae assimilated 38% to 60% of the P applied with the microalgal biomass. The plants grown in algae-amended potting mixes were equivalent to those grown with comparable levels of fertilizer-amended potting mixes with respect to seedling dry weight and nutrient content.

An added benefit of algal biomass is that it does not need to be tilled into soil, which is generally necessary for mineral P fertilizers. Algal biomass may be side-dressed into growing crops, thereby saving labour and energy.

As explained in Alvarez, A. L., Weyers, S. L., Goemann, H. M., Peyton, B. M. and Gardner, R. D. (2021) “Microalgae, soil and plants: A critical review of microalgae as renewable resources for agriculture”. Algal Research, 54, 102200, the diverse effects that microalgal biomass (or microalgal compounds) have on soils and plants, and the different mechanisms of action, offer the opportunity to potentially derive multiple agricultural products from microalgae with applications for soil improvement and crop production and protection.

For example, in addition to use as biofertilizer (whether provided in viable or non-living form—e.g. oven-dried) when applied to soil (micro-algal soil amendment), the microalgal biomass can improve physical properties such as soil structure and water retention, and therefore one of the potential applications is as soil conditioners.

In addition, microalgae may have utility as plant biostimulants, biopesticides or biocontrol agents.

In a further aspect there is provided use of the microalga strain-based fertiliser as an agricultural fertiliser e.g. a method of increasing the P availability in an environment (and optionally improving one or more of the other properties discussed above) by dispersing the strain-based fertiliser in the environment, for example to grow crops or other plants.

### Definitions

“Nucleic acid” according to the present invention may include cDNA, RNA or genomic DNA. Where a DNA sequence is specified, e.g. with reference to a figure, unless context requires otherwise the RNA equivalent, with U substituted for T where it occurs, is encompassed. Nucleic acids may include more than one nucleic acid molecule. Nucleic acid molecules according to the present invention may be provided isolated and/or purified from their natural environment, in substantially pure or homogeneous form, or free or substantially free of other nucleic acids of the species of origin, and double or single stranded. Where used herein, the term “isolated” encompasses all of these possibilities. The nucleic acid molecules may be wholly or partially synthetic. In particular they may be recombinant in that nucleic acid sequences which are not found together in nature (do not run contiguously) have been ligated or otherwise combined artificially. Nucleic acids may comprise, consist, or consist essentially of, any of the sequences discussed hereinafter.

The “complement” of a nucleic acid described herein means the complementary sequence of the or a nucleotide sequence comprised by the nucleic acid. Optionally complementary sequences are full length compared to the reference nucleotide sequence.

By “promoter” is meant a sequence of nucleotides from which transcription may be initiated of DNA operably linked downstream (i.e. in the 3′ direction on the sense strand of double-stranded DNA).

“Operably linked” means joined as part of the same nucleic acid molecule, suitably positioned and oriented for transcription to be initiated from the promoter. DNA operably linked to a promoter is “under transcriptional initiation regulation” of the promoter.

By “endogenous” is meant the native polypeptide (or encoding gene) which originates from the microalgal strain.

The term “heterologous” is used broadly herein to indicate that the gene/sequence of nucleotides in question have been introduced into said cells of the host or an ancestor thereof, using genetic engineering, i.e. by human intervention. “Heterologous” (or “exogenous”, the terms are used interchangeably). Nucleic acid heterologous to a host cell will be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homologue is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

“Transformed” in this context means that the nucleotide sequences of the heterologous nucleic acid alter one or more of the cell's characteristics and hence phenotype e.g. with respect to PRE efficiency. Such transformation may be transient or stable.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1. Knock-out of CrPTC1 confers high P removal capacity without compromising cell growth. (A) Growth of CC-4533 and the Crptc1 mutant strains in the TAP (with Pi supply) and TA (without Pi supply) mediums. Colonies from left to right are a series of dilutions. The right panel shows the growth curves of CC-4533 and the Crpsr1 mutant under Pi supply (+P) and Pi deprivation (−P) conditions. (B) Total P and polyP content of CC-4533 and the Crptc1 mutant. (C) Assessment of P removal ability of CC-4533 and the Crptc1 mutant with 1 mM Pi supply. (D) Principal component analysis (PCA) shows the global similarity and divergence of transcriptome data. The first two components are shown in the plot. (E) Gene ontology (GO) enrichment analysis of significantly up-regulated genes in the Crptc1 mutant under −P condition. GO terms are highly enriched in ion transport-related terms. GO: 0006817 is P transport. (F) Heatmap of expression profiles of genes involved in P homeostasis under +P and −P conditions.

FIG. 2. Over-expression of PSR1 confers high P removal capacity. (A) Relative expression levels of PSR1 and PTB2 of three representative PSR1-OE lines. (B) Total P and ployP contents in the PSR1-OE lines. (C) P removal ability of the PSR1-OE lines with 1 mM Pi supply. (D) Growth of CC-4533 and the PSR1-OE14 line in the TAP and TA mediums. Colonies from left to right are a series of dilutions. The right panel shows growth curves of CC-4533 and the PSR1-OE14 line under 1 mM Pi supply conditions.

FIG. 3. Over-expression of PSR1 in the Crptc1 mutant enhances P removal of the Crptc1 mutant. (A) Relative expression levels of PSR1 and PTB2 of three representative SPAO lines. (B) Total P and ployP contents in the SPAO lines. (C) Assessment of P removal capacity of the SPAO lines under 1 mM Pi supply. (D) Growth of CC-4533 and the SPAO24 line in the TAP and TA mediums. Colonies from left to right are a series of dilutions. The right panel shows growth curves of CC-4533 and the SPAO24 line under 1 mM Pi supply conditions. (E) Correlation of P removal efficiency (PRE) and relative expression of PSR1 under backgrounds of CC-4533 (blue) and the Crptc1 mutant (red). PRE results were calculated at 48 h under the 1 mM Pi supply condition. (F) SPAO24 showed the highest polyP accumulation and slowest relative polyP reduction upon P deprivation. The left panel shows representative pseudo-color images of cellular polyP stained with DAPI. Bar, 10 μm. Experiments were repeated three times with similar results. The right panel shows the relative polyP contents of different strains under P deprivation treatment.

FIG. 4. SPAO shows a high P removal ability under different simulated conditions and proposed model for SPAO design. Evaluation of P removal ability of CC-4533, the Crptc1 mutant, the PSR1-OE14 line, and SPAO24 line in synthetic aquacultural wastewater (SAWW). (B) Proposed model for SPAO design. Compared to conventional PAO (wildtype microalgae), improved PAO presents higher polyP accumulation and higher P removal capacity. Three improving approaches for genetic engineering of improved PAO are suggested: 1) genetic operation of genes controlling the vacuolar P homeostasis. Down-regulation (or loss-of-function) of SPX-SLC proteins could raise the P accumulation in vacuoles and further increase the P removal capacity in improved PAO; 2) increase the expression of PSR1, which further promotes Pi acquisition through directly up-regulating the expression of P starvation-induced genes (PSIGs); 3) the best way-combining above two approaches-enhancing P starvation signalling and trapping P into vacuoles and generating the SPAO strains which showed the highest PRE and highest polyP accumulation.

FIG. 5. Large-scale culture of SPAO24 and CC-4533 in 1 L, 2 L and 8 L medium.
(A-C) Extended culture of SPAO24 and CC-4533 in 1 L, 2 L and 8 L medium. Photos taken at 1 day after inoculation.
(D-E) Total P content in medium and OD750 detected at 40 and 60 hours after inoculation into 2 L medium.

FIG. 6. Example Microalgae-based wastewater treatment (MBWT) process.

Figure taken from Li, K., Liu, Q., Fang, F., et al. (2019) Microalgae-based wastewater treatment for nutrients recovery: A review. Bioresource Technology, 291, 121934).

FIG. 7. Examples of designs and configurations of MBWT processes.

(a) SB-MPBR, (b) twin-layer (TL) PBR, (c) air-lift (AL) PBR, (d) RABR-enhanced RP, (e) RABR, (f) multilayer PBR (from Li et al, supra)

FIG. 8. Schematic of algal biofilm reactor (ABR).

A side view; B front view; L: Length of growth surface B: width of growth surface (from Choudhary, P., Prajapati, S. K., Kumar, P., Malik, A. and Pant, K. K. (2017) Development and performance evaluation of an algal biofilm reactor for treatment of multiple wastewaters and characterization of biomass for diverse applications. Bioresource Technology, 224, 276-284).

FIG. 9. Schematic diagram of MBR setup.

Equipped with Chlorella encapsulated macrocapsules (a) and free Chlorella cells (b) (from Qin, L., Gao, M., Zhang, M., Feng, L., Liu, Q. and Zhang, G. (2020) Application of encapsulated algae into MBR for high-ammonia nitrogen wastewater treatment and biofouling control. Water Research, 187, 116430).

FIG. 10 Over-expression of PSR1 in the Crptc1 mutant further enhanced phosphorus (P) removal and cellular accumulation.

(a) Total P concentrations of CC-4533 and SPAO23 strains cultured in the medium with different P inputs. Error bars indicate SE. (b) Total P concentration of land plants and algae. Data on the total P concentration of land plants were collected from the previous study (Reich, P. B. and Oleksyn, J. (2004) Global patterns of plant leaf N and P in relation to temperature and latitude. Proc. Natl. Acad. Sci., 101, 11001-11006) and from measurements in this study.

FIG. 11 Evaluation of P-removal capacity of the CC-4533 and SPAO23 lines with actual industrial wastewater.

Error bars indicate SE.

### EXAMPLE 1—SUMMARY OF INVESTIGATION

We have investigated whether it is possible to engineer one or more genes involved in cellular P homeostasis in algae to generate improved algae (termed super PAO (“SPAO”) here) with higher efficiency of P luxury uptake and higher P accumulation capacity.

We tested three engineering strategies following:
1) knocking out CrPTC1 to restrict polyP into vacuoles;
2) over-expression of PSR1 to trigger excessive global P starvation signalling, and
3) a combination of these two approaches-over-expression of PSR1 in the Crptc1 mutant.

We then conducted a detailed assessment of the engineered strains and employed them to recycle P from simulated wastewater. We found that all these three kinds of engineered strains have a higher ability to remove P from the environment without compromising biomass production compared to wildtype, but that the third type showed particularly unexpected benefits.

### EXAMPLE 2—INCREASING ACCUMULATION OF POLYP IN VACUOLES

Given that excess Pi is stored as polyP in algae vacuoles (also called acidocalcisomes) (14), we investigated whether we could modulate vacuole-located P transporters to increase the accumulation of polyP in vacuoles to further improve luxury P uptake

In our previous study, CrPTC1 was shown to be involved in cellular P homeostasis, and loss-of-function of CrPTC1 caused the excess P and polyP accumulation in acidocalcisomes (13), indicating that the Crptc1 mutant is a potential improved PAO.

In the design of engineered microalgae, an efficient PAO is expected to have a high P removal ability without compromising cell viability under either P sufficient or P deficient conditions (6).

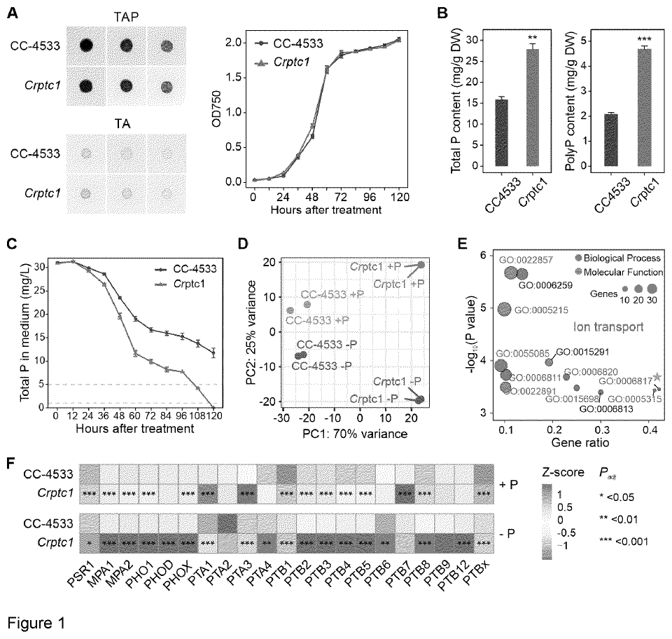

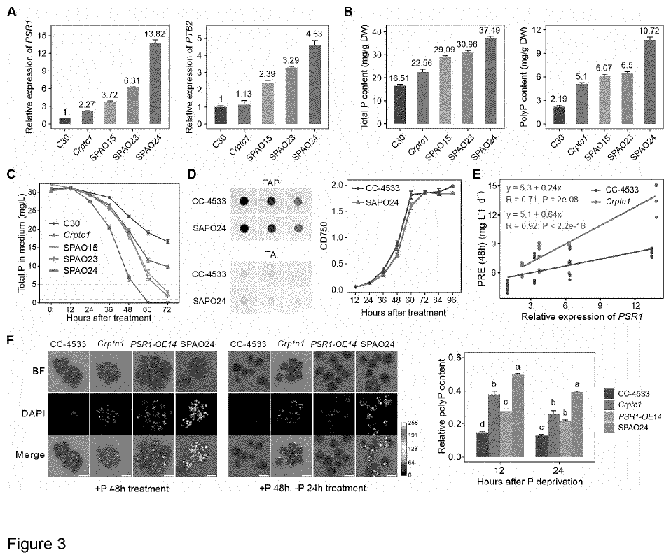

To test this, we first evaluated its physiological status under both Pi sufficient and deficient conditions (FIG. 1A). Given that the Crptc1 mutant accumulates more P and polyP, we hypothesized that the Crptc1 mutant should be less sensitive to low Pi stress. Therefore, we investigated the growth under P-replete or depleted conditions. Like the WT, the Crptc1-1 mutant grew less on the P-depleted condition than on the P-replete condition but showed no growth defect compared to the wildtype in both conditions (FIG. 1A). The total P content and polyP contents in the Crptc1 mutant are significantly higher (around two times) than in the wild-type strain (CC-4533) (FIG. 1B). These data show that the Crptc1 mutant is less sensitive to Pi deficiency stress and could accumulate more P in the cells under both P-replete and depleted conditions, suggesting that the Crptc1 mutant has the potential to remove P from external environments. We then assessed the P removal ability of the Crptc1 mutant by simulating the wastewater environment through an external 1 mM Pi supply (FIG. 3C). After 120 hours, the Crptc1 mutant could remove nearly all Pi in the medium, while the wildtype line could only remove around 62% Pi, leaving a final Pi concentration of 11.72 mg L⁻¹ in the medium. Together, Crptc1 has a high potential for P removal, regardless of whether Pi or organophosphorus is present in the external environment.

### EXAMPLE 3—DISSECTING THE GENE REGULATORY NETWORK UPON PI STARVATION AND ASSESSING THE EFFECT OF CRPTC1 ON P HOMEOSTASIS AND INCREASED ACCUMULATION OF POLYP IN VACUOLES

We analyzed gene expression profiles of the Crptc1 mutant after six hours of Pi starvation treatment using RNA-seq. Principal component analysis (PCA) shows the global similarity of bio-replicates for each treatment and significant differences in expression profiles between the Crptc1 mutant and CC-4533 either under Pi sufficiency or Pi deficiency conditions (FIG. 1D). Gene ontology (GO) enrichment analysis of significantly up-regulated genes in the Crptc1 mutant under P starvation shows that terms related to ion transport are enriched considerably (FIG. 1E). Among them, the annotation with the largest gene ratio is enriched in phosphate ion transport (GO: 0006817). Notably, genes involved in Pi homeostasis are dramatically up-regulated, including the core regulator PSR1 and some genes of several well-known Pi-signalling related gene families, such as phosphatase, PTA, and PTB families (FIG. 1F). These results indicate that loss-of-function of CrPTC1 caused the over-accumulation of polyP in vacuoles and induced P starvation signalling to promote P uptake further.

### EXAMPLE 4—EFFECT OF MODULATION OF EXPRESSION OF THE CORE REGULATOR PSR1 IN ALGAE

We speculated that increasing the expression of the core regulator PSR1 in algae might increase P removal directly.

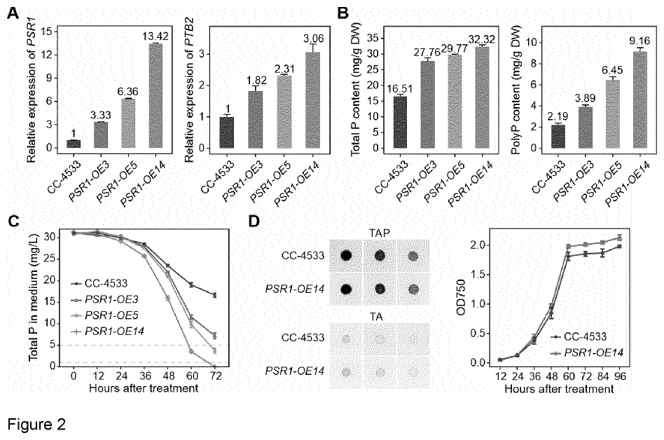

To test this, we developed three PSR1 over-expression (termed PSR1-OE) lines with different expression levels of PSR1 (FIG. 2A) and further evaluated their physiological characters and P entrapment potential. All three representative PSR1-OE lines showed higher expression of PSR1 than wildtype, up to more than 13.4 times. The relative expression of a PTB2 is also higher in all PSR1-OE lines, indicating higher P uptake in the PSR1-OE lines (FIG. 2A). Both total P and polyP showed significant elevation in all three PSR1-OE lines (FIG. 2B). Further P removal simulation results show that all PSR1-OE lines show excellent P removal ability (FIG. 2C), indicating that engineering the core regulator PSR1 can enhance the luxury P uptake. Meanwhile, the strain with a higher expression of PSR1 showed a higher P removal efficiency (PRE). Thus, the PSR1-OE14 strain was selected for further analysis, with the highest expression of PSR1. Growth assessment results found no growth defects in the PSR1-OE14, no matter under Pi sufficiency or Pi deficiency conditions (FIG. 2D). The results showed that the PSR1-OE14 strain had a higher PRE than Crptc1.

### EXAMPLE 5—AN ALGAL STRAIN WITH FURTHER ENHANCED PAO PERFORMANCE

The above results show that either inducing P starvation signalling by increasing PSR1 expression or over-accumulation polyP in vacuoles by knock-out CrPTC1 could increase the luxury P uptake. However it could not be predicted how these different approaches may interact, or whether algal strains embodying both approaches would be robust and viable.

Therefore we next attempted to create strains with high expression of PSR1 in the Crptc1 mutant background. All three representative Crptc1: PSR1-OE lines (termed as SPAO hereafter) presented a higher expression of PSR1 than that in wildtype, as well as the expression of PTB2 (FIG. 3A). Also, both total P and polyP showed significant elevation in all three SPAO lines (FIG. 3B). Further P removal simulation results show that all SPAO lines show excellent P removal ability (FIG. 3C). Among them, the SPAO24 line can completely remove P in the medium at 60 h, which is much faster than the PSR-OE14 line (completely removed P at 72 h). Growth assessment results found no growth defects in the SPAO24 line, whether under Pi sufficiency or Pi deficiency conditions (FIG. 3D). In addition, we found that PRE increased with PSR1 expression in different backgrounds, but in the Crptc1 background, the strains with similar PSR expression levels had higher PRE (FIG. 3E), suggesting that trapping P in vacuoles significantly promotes PRE, while the combination of the two approaches gives a more effective interactive strategy. PolyP staining and contents also showed that after 24 P starvation treatment, SPAO24 retained more polyP in vacuoles than the wild type, Crptc1 and PSR1-OE14 lines (FIG. 3F). While PSR1-OE lines showed a substantial reduction in polyP content, indicating that PSR1-OE lines cannot trap P in vacuoles steadily when P in solution decreases to P deficient conditions. Taken together, these data suggest that over-expression of PSR1 in the Crptc1 background can further improve PRE.

### EXAMPLE 6—ASSESSMENT OF ALGAL STRAINS OF THE INVENTION WITH SYNTHETIC AQUACULTURAL WASTEWATER (SAWW)

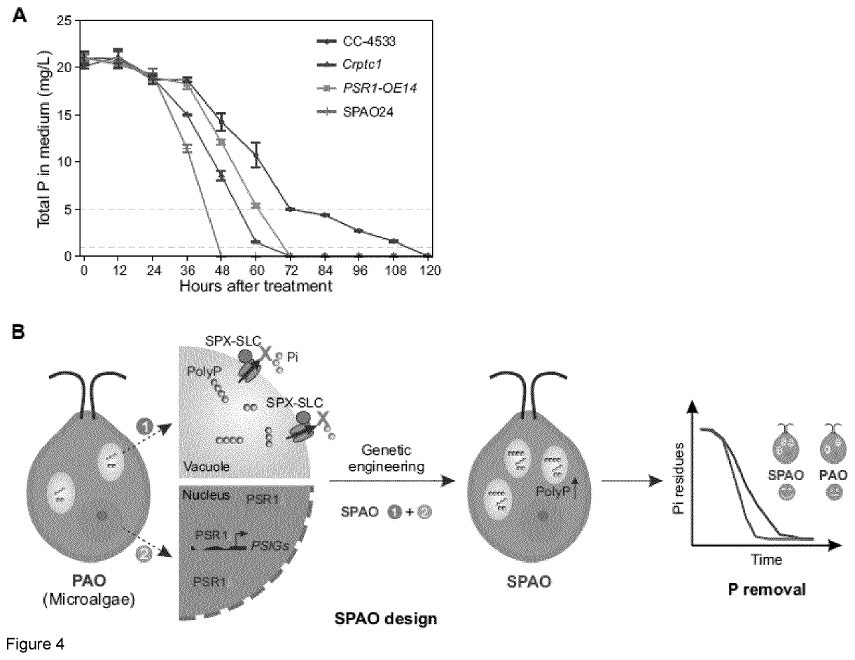

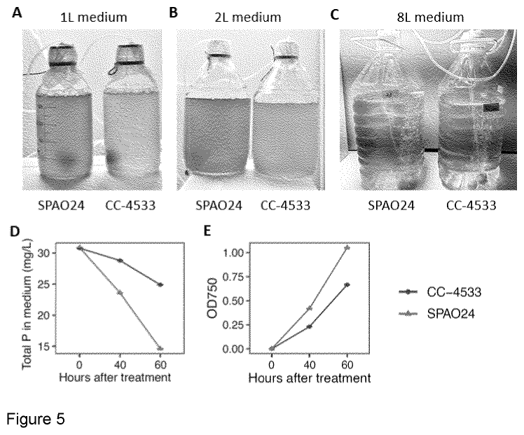

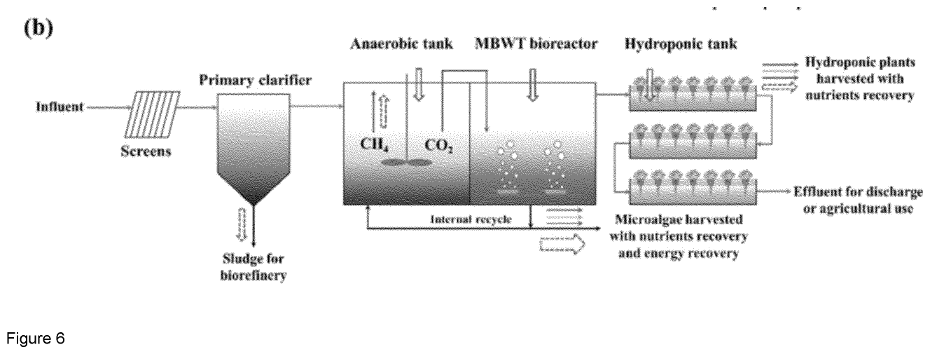

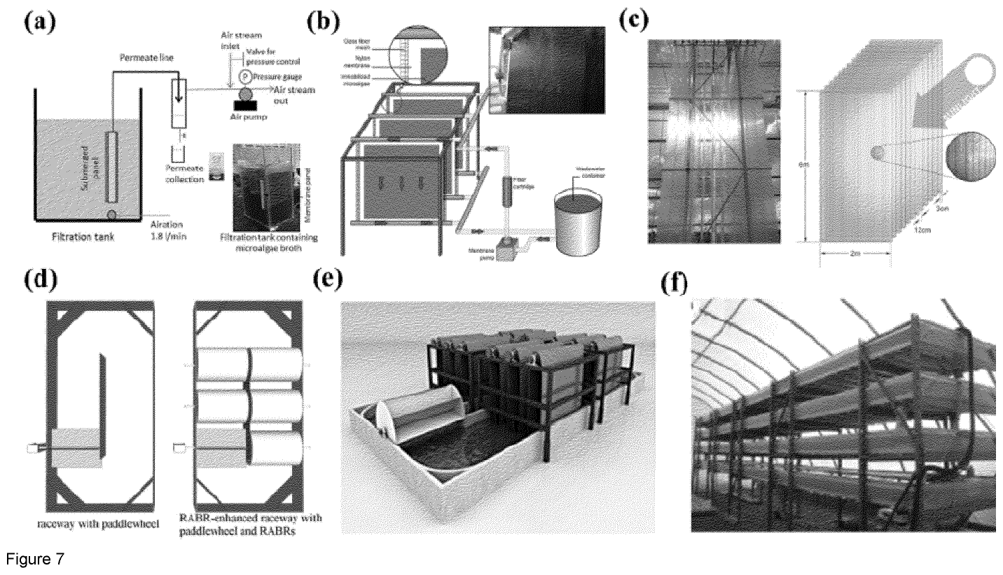

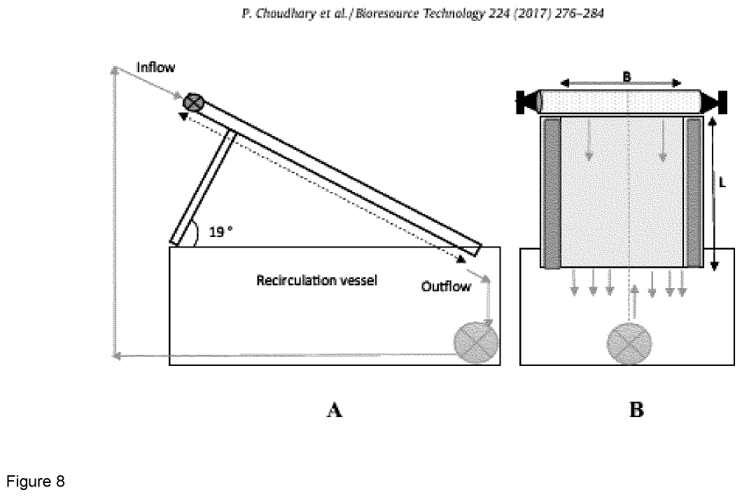

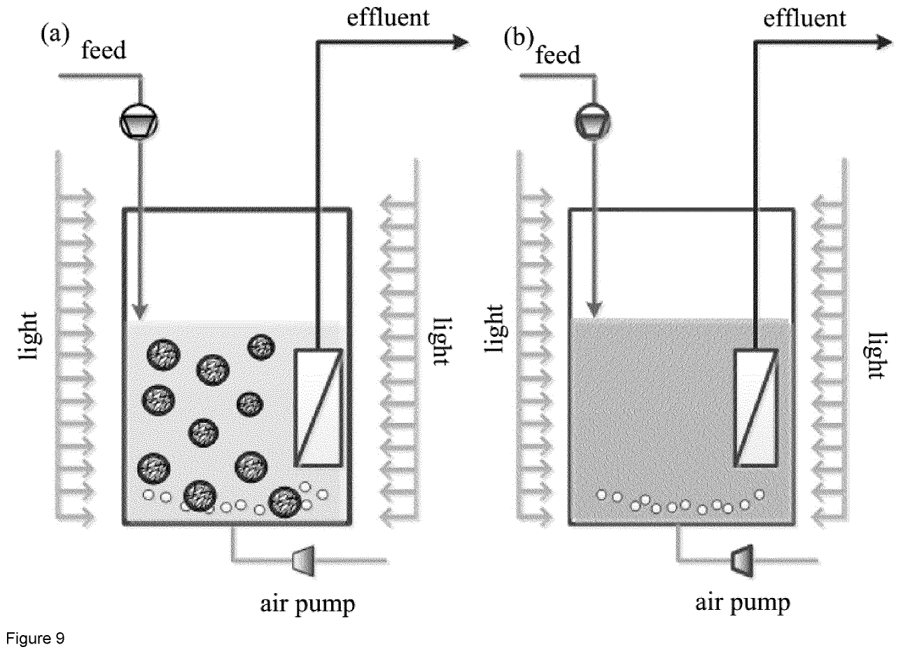

To assess the P removal ability of the strains described above, we further conducted a simulated evaluation of three representative strains of the three approaches, using synthetic aquacultural wastewater (SAWW). These were the Crptc1 mutant, PSR1-OE14 line, and SPAO24 line, along with wildtype CC-4533 as a control. The results showed that all three engineered strains showed higher P removal ability than wild type, and SPAO24 strain showed highest PRE (FIG. 4A). Thus, three improved approaches for genetic engineering of SPAO are proposed (following (FIG. 4B):

1) genetic manipulation of endogenous genes controlling vacuolar P homeostasis. Down-regulation (or loss-of-function) of SPX-SLC proteins can raise the P and polyP accumulation in vacuoles, and further increase the P removal capacity in SPAO.

2) increase the expression of core regulator of P starvation response-PSR1. PSR1 further promotes Pi acquisition through directly up-regulating the expression of P starvation-induced genes (PSIGs), such as phosphate transporters (PTs) which are responsible for Pi absorption from the extracellular environment and alkaline phosphatases (ALPs) which could liberate soluble reactive phosphorus from dissolved organic P compounds.

3) combining above two approaches-enhancing P starvation signaling and trapping P into vacuoles.

### EXAMPLE 7—SCALED UP CULTURE

In the Examples above microalgae culture was carried out at lab-scale (typically 100 to 150 mL medium).

In further experiments we used extended culture in larger volumes (1 L, 2 L, and 10 L) of medium. As shown in FIG. 5, using a same inoculation amount with the previous lab-scale system (about 10⁵ cells/L), the SPAO24 strain showed an growth advantage compared to wild-type strain CC-4533 in all large-scale culture systems within a relatively short time after inoculation (about 1 day).

### EXAMPLE 8—UTILITY OF ALGAE AS FERTILISER

For use as fertiliser the microalgae are recovered and added to fields growing crop plants.

Although higher crop plants can typically take up inorganic phosphate from external environments, the polyPs can be degraded naturally by polyphosphatases, which occur in bacteria and fungi in the natural environment and are reviewed in (Lorenzo-Orts et al., 2020). In brief, in bacteria, long-chain polyPs can be sequentially hydrolyzed by exopolyphosphatase 1 (PPX1). PPX1 belongs to the same protein superfamily as actin, HSP70 chaperones and sugar kinases, and hydrolyzes both polyP and the alarmone guanosine pentaphosphate (pppGpp). The short-chain inorganic polyphosphatase ygiF from Escherichia coli hydrolyzes tripolyphosphate into pyrophosphate and Pi. In yeast, PPX1 belongs to the DHH phosphatase family and hydrolyzes the terminal Pi from short-chain polyPs. Siebers et al., 2019 demonstrates that the P in algae can be rapidly transformed in soil and mobilized for plant growth.

### EXAMPLE 9—ASSESSMENT OF THE MAX CAPACITY OF CELLULAR P IN ENGINEERED ALGAE

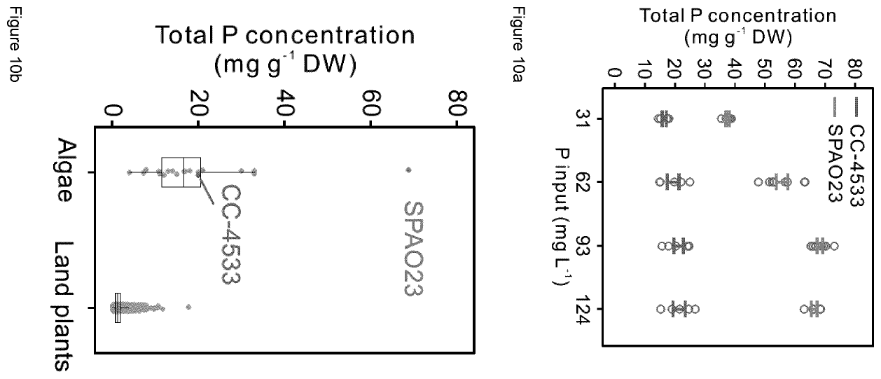

Given that the SPAO23 strain accumulated more than twice as much total P as the WT did under normal culture conditions (FIG. 3b), then, it is asked what is the possible maximum P accumulation capacity in SPAO strains. To assess this, the SPAO23 strain and the WT were cultured in modified TAP conditions amended with different concentrations of Pi. Total P concentrations increased with increasing P input and did no further increase after P input exceeded 93 mg L⁻¹ (3 times of the normal concentration in TAP) (FIG. 10a). At 93 mg L⁻¹ P input, the total P concentration in the SPAO23 line reached a maximum of 68.3 mg g⁻¹ DW, while the total P concentration in WT was relatively stable at about 20 mg g⁻¹ DW (FIG. 10a). Thus, it is proposed that the over-expression of PSR1 in the Crptc1 background can enormously increase maximum P accumulation capacity up to almost 7% dry matter. So far, this is the maximum stoichiometric proportion of total P have to be achieved in plants (FIG. 10b).

### EXAMPLE 10—REMOVING P FROM INDUSTRIAL WASTEWATER

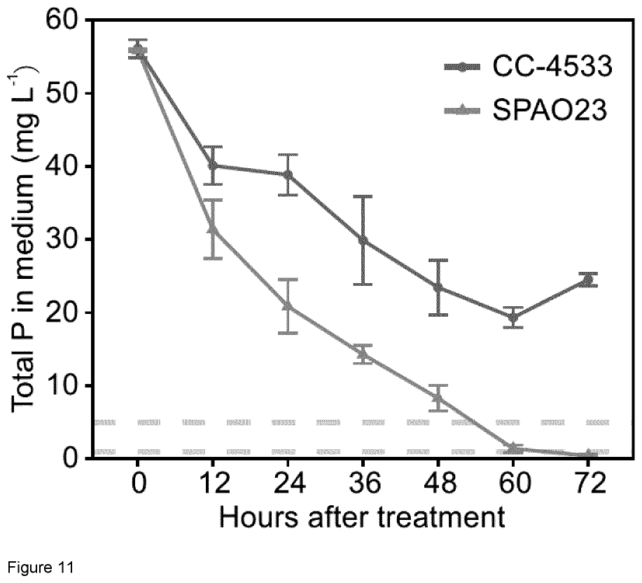

To evaluate the P-removal capacity of the engineered algae strains in the real wastewater environment, the wastewater from a chemical plant in Nantong, China, was collected for further analysis. The wastewater was used in the experiments directly without filtration to minimize any change in water composition. Characteristic analysis showed that the wastewater contained 56 mg L⁻¹ total P and 34 mg L⁻¹ total N, with 1100 mg L⁻¹ chemical oxygen demand (COD). To simulate the actual wastewater treatment scenario as much as possible (Nie, X., Mubashar, M., Zhang, S., Qin, Y., and Zhang, X. (2020) Current progress, challenges and perspectives in microalgae-based nutrient removal for aquaculture waste: A comprehensive review. J. Clean. Prod., 277, 124209), after adjusting the wastewater to the algal growth conditions (details in Methods), an approximately 10% initial inoculum (about 10⁶ cells L-1) of SPAO23 strains and its WT were used to inoculate wastewater in a 50 mL working volume. Measurement of the residual P in the wastewater showed that SPAO23 had removed 97.4% of the total P from the wastewater after 60 h, and it recovered all the P within 72 h (FIG. 11). In contrast, the wastewater inoculated with the WT strain reached the lowest residual P at 60 h (34.5% of initial P concentration), and this even increased with prolonged cultivation (FIG. 11). The above results confirmed that SPAO23 has superior P removal application prospects.

### EXAMPLE 11—CONCLUSIONS FROM EXAMPLES 1-10

The current evidence supports the view that integrating microalgae as an alternative biological wastewater treatment approach is environmentally and technologically feasible (5, 16). Species of several algae genera have been assessed and employed for phosphorus removal capacities from wastewater, such as Chlorella, Scenedesmus, Cyanobacteria, Oocystis, and Ankistrodesmus (16). In this study, we proved that engineering the genes involving the P homeostasis could enhance the luxury P removal and enable the development of species or strains that are more efficient at P removal from wastewater. PSR1 has been shown to be conserved in regulating the P starvation signalling in green plants (17). Our previous study also has demonstrated that SPX-SLC proteins are widely found in green algae, which are responsible for the efflux of vacuolar polyP in green algae (13). These conservative mechanisms of P homeostasis are widespread in green algae. Thus, although this study uses the model green alga-C. reinhardtii as an example, it is credible that analogous methodology may be used to produce other genetically engineered microalgae with enhanced ability to remove P from wastewater.

### Materials and Methods Used in Examples

Chlamydomonas reinhardtii Strains and Growth Conditions

The Chlamydomonas reinhardtii strain CC-4533 (also refers to CMJ030) and Crptc1 (LMJ.RY0402.181899) were purchased from the Chlamydomonas Resource Center (18).

This strain was generated by the CIB1-insertion method as follows:

To generate mutants, cells of the wild-type strain CC-4533 were transformed with DNA cassettes (termed CIB1 cassette) that randomly insert into the genome, confer paromomycin resistance for selection, and inactivate the genes into which they insert. Each cassette contained two unique 22-nucleotide barcodes, one at each end of the cassette. Transformants were arrayed on agar plates, and each insertion in a transformant would contain two barcodes. To make sure the insertion, DNA was then extracted from each pool, and barcodes were amplified and deep-sequenced.

Li, X., Patena, W., Fauser, F., et al. (2019) A genome-wide algal mutant library and functional screen identifies genes required for eukaryotic photosynthesis. Nat Genet, 51, 627-635.

Alternatively, an miRNA targeting Chlamydomonas PTC1 may be provided according to (Molnár et al., 2009) using the WMD3 tool at http://wmd3.weigelworld.org/. Resulting oligonucleotides are annealed by boiling and slowly cooling down in a thermocycler and ligated into Spel-digested miRNA2, yielding miRNA2-PTC. miRNA2-PTC is linearized by digestion with Scal and transformed into Chlamydomonas strain CC-4533 by electroporating (Bio-Rad; Gene Pulser2 electroporation system) with pulse settings of 800 V and 25 uF, followed by immediate decanting into a 15-mL tube containing 13 mL of TAP supplemented with 40 mM sucrose. Cells are then collected by centrifugation at 1000 g for 4 min, with most of the supernatant being decanted, and the cells resuspended in the remaining 500 mL of supernatant. Resuspended cells are gently plated onto 2% (w/v) TAP agar plates containing 20 mg/mL paromomycin. These plates are stored at 5 mmol photons m⁻² s⁻¹ light for 2 weeks, until transformant colonies appear (Molnar, A., Bassett, A., Thuenemann, E., Schwach, F., Karkare, S., Ossowski, S., Weigel, D. and Baulcombe, D. (2009) Highly specific gene silencing by artificial microRNAs in the unicellular alga Chlamydomonas reinhardtii. The Plant Journal, 58, 165-174).

Alternatively, a CRISPR based method may be used via transformation with an RNP complex consisting of LbCpf1 protein and a gRNA targeting a PAM sequence in the first exon of CrPTC1 as described in Ferenczi et al. (2017). Cells were incubated at 40° C. for 20 min. Purified LbCpf1 (80 μM) is preincubated with gRNA (1 nmol) at 25° C. for 20 min to form RNP complexes. For template DNA-mediated editing, ssODN (5.26 nmol) is added at a 1:10 molar ratio to LbCpf1. Final volumes are around 270-280 μL. Cells are electroporated in 4-mm cuvettes (800 V, 25 μF) by using Gene Pulser Xcell (Bio-Rad). 800 μL of TAP with 40 mM sucrose is added immediately after electroporation. Cells are recovered overnight (24 h) in 5 mL TAP with 40 mM sucrose shaken at 110 rpm and then plated onto TAP media supplemented with 10 UM rapamycin (Ferenczi, A., Pyott, D. E., Xipnitou, A. and Molnar, A. (2017) Efficient targeted DNA editing and replacement in Chlamydomonas reinhardtii using Cpf1 ribonucleoproteins and single-stranded DNA. Proceedings of the National Academy of Sciences, 114, 13567-13572).***Cells were cultured in a standard Tris-acetate-phosphate TAP medium at pH 7.0 under continuous illumination (50 mmol photons m⁻² s⁻¹) on a rotating platform (150 rpm) at 24° C. For Pi deprivation, cells in the mid-logarithmic phase (5-8×10⁶ cells mL-1) were pelleted by centrifugation (2,000 g, 5 min), washed twice with TA in which 1.5 mM potassium chloride was substituted for 1 mM potassium phosphate (19), and then resuspended in TA medium.

### Generation of Over-Expression Chlamydomonas Lines

To create CrPSR1 over-expression strains, the genomic DNA of CrPSR1 was introduced into the HSP70-ARbcS2-Ble vector (20), then the reconstructed plasmids were linearized with Scal before electroporation into CC-4533 and the Crptc1 mutant cells. Transformants were selected on the solid TAP medium containing 10 μg mL⁻¹ bleomycin (21). Positive transformants were further validated by relative expression level of PSR1 using qRT-PCR.

Measurement of Total P and polyP

For measurement of total polyP, 0.5 mL cells were harvested (2300 g, 2 min) and the pellet was frozen immediately at −20° C. for later analysis. After thawing, 50 μL of 1 M H₂SO₄ was added to the cells. PolyP was purified using PCR purification columns (22). 5 μL polyP solution was mixed with an equal volume of 2 M HCl and heated at 95° C. for 30 min. The content of Pi released from polyP was measured by the Mo-blue method (86 UL of 28 mM ammonium heptamolybdate in 2.1 M H₂SO₄ and 64 μL of 0.76 mM malachite green in 0.35% polyvinyl alcohol). The absorbance was measured at a wavelength of 595 nm in a TECAN infinite Elisa Reader. For the measurement of total phosphorus concentration, 5 mL cells were harvested and dried. The total P content was measured as previously (23).

Staining PolyP with DAPI

PolyP within cells was stained with DAPI and imaged through a ZEISS LSM 880 scanning confocal microscope. Cells were grown in TAP medium to 6×10⁶ cells mL-1 and incubated with DAPI. DAPI was excited at 405 nm and emission was collected from 532 to 632 nm, similar to conditions previously described (24).

### Quantitative Real-Time PCR Analysis

Total RNA was extracted from frozen cell pellets using the RNeasy Mini Kit (Qiagen) and reverse transcribed to complementary DNA after DNase I treatment following the standard instructions (NEB). Quantitative real-time PCR was performed using a SYBR Premix kit (Roche) on a QuantStudio 6 Flex machine (Life Technologies). The CBLP gene was used as an internal control (26). The primer pairs used for RT-qPCR are given in the Table S1:

TABLE S1
Primers used in this study.

| Primer | | |
|---|---|---|
| names | Sequence (5′-3′) | Use of primers |
| PSR1.F | GGAATTCCATATGTCG | Over-expression line of |
| | CTATGCAACGATCTAC | PSR1 |
| | G | |
| PSR1.R | CCGGATATCGCTGCCG | Over-expression line of |
| | TGAACAGTACAAA | PSR1 |
| PTB2.QF | AGACGGCTGAACAGTG | Quantitative RT-PCR of |
| | CTAC | PTB2 |
| PTB2.QR | CGTGGAGACCCATATG | Quantitative RT-PCR of |
| | ACCG | PTB2 |
| CBLP.QF | CTTCTCGCCCATGACC | Quantitative RT-PCR of |
| | AC | CBLP |
| CBLP.QR | CCCACCAGGTTGTTCT | Quantitative RT-PCR of |
| | TCAG | CBLP |
| PSR1.QF | ACAGCAGCAACAAGAG | Quantitative RT-PCR of |
| | CAAC | PSR1 |
| PSR1.QR | CGAAATCACCGAAGTC | Quantitative RT-PCR of |
| | AAAG | PSR1 |

### Synthetic Aquacultural Wastewater

Synthetic aquaculture wastewater (SAWW) was prepared based on the characteristics of local aquaculture wastewater from Zhoushan, China. The components were the following: ammonium, 120 mg L⁻¹; orthophosphate, 20 mg L⁻¹; and 92.3 mg L⁻¹ of CH3COONa as an additional carbon source. Other nutrients added as the TAP medium. The pH of the synthetic aquaculture wastewater was controlled at approximately 7.

### Wastewater Source and Experimental Setup

Industrial wastewater used in this study was collected from a chemical plant located in Nantong, China, which contains 34 mg L⁻¹ total N and 56 mg L⁻¹ total P, with 2200 mg L⁻¹ chemical oxygen demand (COD). To adjust the wastewater to the algal growth conditions, 3 mM NH₄Cl, 0.2 mM MgSO₄, 0.34 mM CaCl₂), and 0.5 ml L⁻¹ Hunter's Trace Stock Solution (Harris, E. H. (1989) The Chlamydomonas Sourcebook. Elsevier) were added to generate experimental conditions. The pH was controlled at approximately 7.0. The working volume was 50 mL, and the initial inoculum was approximately 10% (about 10⁶ cells L⁻¹).

### Elemental Analysis

For elemental analysis, 10 mL of algal cells were harvested, dried, and then digested with 65% HNO₃ at 75° C. for six hours. The elemental concentration was determined by inductively coupled plasma optical emission spectrometry (ICP-OES, Thermo Scientific, USA). The results are given in the Table S2:

TABLE S2
Elemental analysis of CC-4533 and SPAO23.

| Elements (ppm) | CC-4533 | SPAO23 |
|---|---|---|
| Ca | 4.199 ± 0.158 | 7.068 ± 0.211** |
| Cu | 0.019 ± 0.007 | 0.017 ± 0.004 |
| Fe | 0.453 ± 0.062 | 0.464 ± 0.106 |
| K | 3.308 ± 0.124 | 3.796 ± 0.092 |
| Mg | 4.366 ± 0.408 | 6.247 ± 0.274** |
| Mn | 0.216 ± 0.023 | 0.204 ± 0.008 |
| Na | 0.706 ± 0.051 | 0.646 ± 0.055 |
| S | 5.669 ± 0.480 | 4.804 ± 0.186 |
| Ti | 4.518 ± 0.804 | 3.313 ± 1.032 |
| Zn | 0.064 ± 0.007 | 0.050 ± 0.006 |

### RNA Sequencing and Data Analysis

Total RNA was extracted by TaKaRa MiniBEST Universal RNA Extraction Kit, and at least two independent biological replicates were used for each line. Library construction of RNA and sequencing was carried out by HiSeq 4000 platform with paired-end (2×150 bp) sequencing. Transcriptome data were prepared as described in our previous study (27). Briefly, the paired-end reads for each individual were mapped against the Chlamydomonas reinhardtii reference genome (JGI v5.5) using HISAT2 (version 2.1.0) (28). FPKM (fragments per kilobase of exon model per million reads mapped) and TPM (transcripts per million) values were calculated by StringTie (version 1.3.4b) with default parameters (29). Differential expression analysis was carried out by DESeq2 (30). Z-score value of each gene was calculated by Mfuzz (31). Significant changes in differentially expressed genes (DEGs) were determined as fold-change more than 2 and fold-change less than 0.5 for up-regulation and down-regulation respectively, with P value <0.05. Gene ontology (GO) analysis was performed using agriGO v2.0 (32). Significantly enriched GO items were filtered by P value <0.01 and false discovery rate (FDR)<0.05. Diagrams were drawn by R scripts available by request.

### REFERENCES

1. D. Cordell, J.-O. Drangert, S. White, Glob. Environ. Change. 19, 292-305 (2009).
2. G. K. MacDonald, E. M. Bennett, P. A. Potter, N. Ramankutty, Proc. Natl. Acad. Sci. 108, 3086-3091 (2011).
3. D. Cordell, S. White, Annu. Rev. Environ. Resour. 39, 161-188 (2014).
4. D. W. Schindler et al., Proc. Natl. Acad. Sci. 105, 11254-11258 (2008).
5. K. Li et al., Bioresour. Technol. 291, 121934 (2019).
6. Y. Xu, Y. Wu, S. Esquivel-Elizondo, J. Dolfing, B. E. Rittmann, Trends Biotechnol. 38, 1292-1303 (2020).
7. A. Solovchenko, A. M. Verschoor, N. D. Jablonowski, L. Nedbal, Biotechnol. Adv. 34, 550-564 (2016).
8. L. Qin et al., Water Res. 187, 116430 (2020).
9. I. S. A. Abeysiriwardana-Arachchige, H. M. K. Delanka-Pedige, S. P. Munasinghe-Arachchige, N. Nirmalakhandan, Bioresour. Technol. 332, 125128 (2021).
10. D. D. Wykoff, A. R. Grossman, D. P. Weeks, H. Usuda, K. Shimogawara, Proc. Natl. Acad. Sci. 96, 15336-15341 (1999).
11. J. L. Moseley, C.-W. Chang, A. R. Grossman, Eukaryot. Cell. 5, 26-44 (2006).
12. A. K. Bajhaiya, A. P. Dean, L. A. H. Zeef, R. E. Webster, J. K. Pittman, Plant Physiol. 170, 1216-1234 (2016).
13. L. Wang et al., Mol. Plant. 14, 838-846 (2021).
14. M. Aksoy, W. Pootakham, A. R. Grossman, Plant Cell. 26, 4214-4229 (2014).
15. F. Wollmann et al., Eng. Life Sci. 19, 860-871 (2019).
16. S. F. Mohsenpour, S. Hennige, N. Willoughby, A. Adeloye, T. Gutierrez, Sci. Total Environ. 752, 142168 (2021).
17. V. Rubio et al., Genes Dev. 15, 2122-2133 (2001).
18. X. Li et al., Nat. Genet. 51, 627-635 (2019).
19. J. D. Quisel, D. D. Wykoff, A. R. Grossman, Plant Physiol. 111, 839-848 (1996).
20. W. Zhang et al., Proc. Natl. Acad. Sci. 118, e2104443118 (2021).
21. K. Shimogawara, S. Fujiwara, A. Grossman, H. Usuda, Genetics. 148, 1821-1828 (1998).
22. T. P. Werner, N. Amrhein, F. M. Freimoser, Arch. Microbiol. 184, 129-136 (2005).
23. A. Chen, J. Hu, S. Sun, G. Xu, New Phytol. 173, 817-831 (2007).
24. R. Aschar-Sobbi et al., J. Fluoresc. 18, 859-866 (2008).
25. F.-J. Li, C. Y. He, Autophagy. 10, 1978-1988 (2014).
26. C.-W. Chang, J. L. Moseley, D. Wykoff, A. R. Grossman, Plant Physiol. 138, 319-329 (2005).
27. X. Jia et al., J. Plant Physiol. 248, 153141 (2020).
28. D. Kim, B. Langmead, S. L. Salzberg, Nat. Methods. 12, 357-360 (2015).
29. M. Pertea et al., Nat. Biotechnol. 33, 290-295 (2015).
30. M. I. Love, W. Huber, S. Anders, Genome Biol. 15, 1-21 (2014).
31. L. Kumar, M. E. Futschik, Bioinformation. 2, 5-7 (2007).
32. T. Tian et al., Nucleic Acids Res. 45, W122-W129 (2017).

### ADDITIONAL REFERENCES

Acevedo, B., Oehmen, A., Carvalho, G., Seco, A., Borrás, L., and Barat, R. (2012). Metabolic shift of polyphosphate-accumulating organisms with different levels of polyphosphate storage. Water Res. 46:1889-1900.
Chen, X., Li, Z., He, N., Zheng, Y., Li, H., Wang, H., Wang, Y., Lu, Y., Li, Q., and Peng, Y. (2018). Nitrogen and phosphorus removal from anaerobically digested wastewater by microalgae cultured in a novel membrane photobioreactor. Biotechnol. Biofuels 11:190.
Qin, L., Gao, M., Zhang, M., Feng, L., Liu, Q., and Zhang, G. (2020). Application of encapsulated algae into MBR for high-ammonia nitrogen wastewater treatment and biofouling control. Water Res. 187:116430. (=8 above)
Lorenzo-Orts, L., Couto, D., and Hothorn, M. (2020). Identity and functions of inorganic and inositol polyphosphates in plants. New Phytol. 225:637-652.
Siebers, N., Hofmann, D., Schiedung, H., Landsrath, A., Ackermann, B., Gao, L., Mojzeš, P., Jablonowski, N. D., Nedbal, L., and Amelung, W. (2019). Towards phosphorus recycling for agriculture by algae: Soil incubation and rhizotron studies using 33P-labeled microalgal biomass. Algal Res. 43:101634.

TABLE 1
Non-limiting host species of the invention

| | PSR | PSR | PTC | PTC | | | |
|---|---|---|---|---|---|---|---|
| | protein | CDS | protein | CDS | | | |
| | SEQ | SEQ | SEQ | SEQ | | | |
| | ID | ID | ID | ID | | PSR genome sequence | PTC genome sequence |
| Microalgal species | NO: | NO: | NO: | NO: | Source* | accession. | accession. |
| 1 Chlamydomonas_reinhardtii | 1 | 2 | 3 | 4 | NCBI | XM_001700501 | XM_043062609 |
| 2 Monoraphidium_neglectum | 5 | 48 | 91 | 134 | NCBI | XM_014050785 | XM_014038471 |
| 3 Entransia_fimbriata | 6 | 49 | 92 | 135 | OneKP | scaffold-BFIK-2030547 | scaffold-BFIK-2030241 |
| 4 Golenkinia_longispicula | 7 | 50 | 93 | 136 | OneKP | scaffold-BZSH-2001091 | scaffold-BZSH-2006448 |
| 5 Oedogonium_cardiacum | 8 | 51 | 94 | 137 | OneKP | scaffold-DVYE-2079230 | scaffold-DVYE-2077838 |
| 6 Cylindrocapsa_geminella | 9 | 52 | 95 | 138 | OneKP | scaffold-DZPJ-2052897 | scaffold-DZPJ-2003776 |
| 7 Scherffelia_dubia | 10 | 53 | 96 | 139 | OneKP | scaffold-FMVB-2037579 | scaffold-FMVB-2001145 |
| 8 Hafniomonas_reticulata | 11 | 54 | 97 | 140 | OneKP | scaffold-FXHG-2081405 | scaffold-FXHG-2003842 |
| 9 Tetraselmis_chui | 12 | 55 | 98 | 141 | OneKP | scaffold-HVNO-2004729 | scaffold-HVNO-2062440 |
| 10 Volvox_globator | 13 | 56 | 99 | 142 | OneKP | scaffold-ISPU-2045281 | scaffold-ISPU-2049066 |
| 11 Volvox_aureus-M1028 | 14 | 57 | 100 | 143 | OneKP | scaffold-JWGT-2006823 | scaffold-JWGT-2063294 |
| 12 Ignatius_tetrasporus | 15 | 58 | 101 | 144 | OneKP | scaffold-KADG-2046538 | scaffold-KADG-2001949 |
| 13 Gonium_pectorale | 16 | 59 | 102 | 145 | OneKP | scaffold-KUJU-2047796 | scaffold-KUJU-2052195 |
| 14 Planophila_terrestris | 17 | 60 | 103 | 146 | OneKP | scaffold-LETF-2022328 | scaffold-LETF-2024633 |
| 15 Pteromonas_angulosa | 18 | 61 | 104 | 147 | OneKP | scaffold-LNIL-2007754 | scaffold-LNIL-2008234 |
| 16 Asteromonas_gracilis | 19 | 62 | 105 | 148 | OneKP | scaffold-MNPL-2049621 | scaffold-MNPL-2001370 |
| 17 Haematococcus_pluvialis-B | 20 | 63 | 106 | 149 | OneKP | scaffold-ODXI-2004725 | scaffold-ODXI-2081911 |
| 18 Chlamydomonas_bilatus-B | 21 | 64 | 107 | 150 | OneKP | scaffold-OVHR-3009713 | scaffold-OVHR-3004312 |
| 19 Vitreochlamys_sp | 22 | 65 | 108 | 151 | OneKP | scaffold-QWRA-2010672 | scaffold-QWRA-2058512 |
| 20 Botryococcus_terribilis | 23 | 66 | 109 | 152 | OneKP | scaffold-QYXY-2003986 | scaffold-QYXY-2007561 |
| 21 Eudorina_elegans | 24 | 67 | 110 | 153 | OneKP | scaffold-RNAT-2008570 | scaffold-RNAT-2006695 |
| 22 Pandorina_morum | 25 | 68 | 111 | 154 | OneKP | scaffold-RYJX-2054172 | scaffold-RYJX-2002422 |
| 23 Oedogonium_foveolatum | 26 | 69 | 112 | 155 | OneKP | scaffold-SDPC-2070697 | scaffold-SDPC-2071408 |
| 24 Chlamydomonas_sp.-M2762 | 27 | 70 | 113 | 156 | OneKP | scaffold-TSBQ-2014277 | scaffold-TSBQ-2002581 |
| 25 Chlamydomonas_sp.-M2762.PSR | 28 | 71 | — | — | OneKP | scaffold-TSBQ-2014095 | — |
| homologue | | | | | | | |
| 26 Chlamydomonas_noctigama | 29 | 72 | 114 | 157 | OneKP | scaffold-VALZ-2054316 | scaffold-VALZ-2007695 |
| 27 Carteria_crucifera | 30 | 73 | 115 | 158 | OneKP | scaffold-VIAU-2010027 | scaffold-VIAU-2008242 |
| 28 Volvox_aureus-M2242 | 31 | 74 | 116 | 159 | OneKP | scaffold-WRSL-2007027 | scaffold-WRSL-2061065 |
| 29 Phacotus_lenticularis | 32 | 75 | 117 | 160 | OneKP | scaffold-ZIVZ-2051918 | scaffold-ZIVZ-2051878 |
| 30 Stephanosphaera_pluvialis | 33 | 76 | 118 | 161 | OneKP | scaffold-ZLQE-2007917 | scaffold-ZLQE-2028596 |
| 31 Chlamydomonas_eustigma | 34 | 77 | 119 | 162 | PhycoCosm | jgi\|Chleu1\|2902 | jgi\|Chleu1\|8327 |
| 32 Chlamydomonas_incerta | 35 | 78 | 120 | 163 | PhycoCosm | jgi\|Chlin1\|9537 | jgi\|Chlin1\|8443 |
| 33 Chlamydomonas_schloesseri | 36 | 79 | 121 | 164 | PhycoCosm | jgi\|Chlsc1\|11577 | jgi\|Chlsc1\|10888 |
| 34 Chromochloris_zofingiensis | 37 | 80 | 122 | 165 | PhycoCosm | jgi\|Chrzof1\|7509 | jgi\|Chrzof1\|5011 |
| 35 Coccomyxa_subellipsoidea | 38 | 81 | 123 | 166 | PhycoCosm | jgi\|Coc_C169_1\|61415 | jgi\|Coc_C169_1\|40418 |
| 36 Coccomyxa_subellipsoidea PTC | — | — | 124 | 167 | PhycoCosm | | jgi\|Coc_C169_1\|61319 |
| homologue | | | | | | | |
| 37 Symbiochloris_reticulata | 39 | 82 | 125 | 168 | PhycoCosm | jgi\|Dicre1\|239710 | jgi\|Dicre1\|741512 |
| 38 Edaphochlamys_debaryana | 40 | 83 | 126 | 169 | PhycoCosm | jgi\|Edade1\|14722 | jgi\|Edade1\|2330 |
| 39 Enallax_costatus | 41 | 84 | 127 | 170 | PhycoCosm | jgi\|Enacos1_1\|6437860 | jgi\|Enacos1_1\|6401798 |
| 40 Mesostigma_viride | 42 | 85 | 128 | 171 | PhycoCosm | jgi\|Mesvir1\|525 | jgi\|Mesvir1\|25557 |
| 41 Raphidocelis_subcapitata | 43 | 86 | 129 | 172 | PhycoCosm | jgi\|Rapsub1\|10603 | jgi\|Rapsub1\|7807 |
| 42 Symbiochloris_reticulata_Africa | 44 | 87 | 130 | 173 | PhycoCosm | jgi\|SymretAf1\|595148 | jgi\|SymretAf1\|539201 |
| 43 Tetradesmus_deserticola | 45 | 88 | 131 | 174 | PhycoCosm | jgi\|TetrdesSNI2_1\|7399393 | jgi\|TetrdesSNI2_1 \|7661652 |
| 44 Tetraselmis_striata | 46 | 89 | 132 | 175 | PhycoCosm | jgi\|Tetstr1\|445010 | jgi\|Tetstr1\|443528 |
| 45 Trebouxia_sp. | 47 | 90 | 133 | 176 | PhycoCosm | jgi\|TrebA12_1\|9605 | jgi\|TrebA12_1\|7379 |

*NCBI https://www.ncbi.nlm.nih.gov/nucleotide
OneKP https://db.cngb.org/onekp
PhycoCosm https://phycocosm.jgi.doe.gov/phycocosm

Sequence Annex

>Chlamydomonas_reinhardtii.PSR1
MDKAERAAGGPNAASEDDWLLEFWPEPAADFPAPVAPMLSQHQDAAQLPEAMPQQQGLALGGYGLTQQPSDFMQTGMPGFDAF
SSGKAATLGLPLLADPQRASTDGASALMNAAQQSSEYMLAPGMGGMPHLLAPSVGTALPGTGHTGFADLSMGGMAGGIPGLGG
PGIMHGQYFMQPQRAATGPAKSRLRWTPELHNRFVNAVNSLGGPDKATPKGILKLMGVDGLTIYHIKSHLQKYRLNIRLPGES
GLAGDSADGSDGERSDGEGGVRRATSLERADTMSGMAGGAAAALGRAGGTPGGALISPGLAGGTSSTGGMAAGGGGGGGLVTE
PSISRGTVLNAAGAVATAAPAAAAPAGGSAAVKRPAGTSLSSGSTASATRRNLEEALLFQMELQKKLHEQLETQRQLQLSLEA
HGRYIASLMEQEGLTSRLPELSGGAPAAAPVAAGGAAGGMIAPPPPQQQLQHQPQLLQPQGSLPAGGSSEAHAAAGAGTMVVH
QQQQQHVHHHHQQQQVQMQQHARHCDTCGAGGAGGAPSGGSSMQQLQAAEQQRTELVVAGRLGSMPAPASSSPLAGQAHQQQP
LAGGAAHLVHVHSHTPGGQPHVQHQDAFAGAATAAAHASPGLPQSHSHLLPADLSSNAGPDTSAGQIKPEPDMSQQQQQQEQQ
EAEQLAQGLLNDSSAGAGAVSGSDGGGLGDFDFGDFGDLDGGAQGGLLGPGDLIGIAELEAAAAHEQQQEQEHDPLDADRAKR
QRVEP
>Monoraphidium_neglectum.PSR1
MQQDLLGGPPGPMHHDEQHEMLQQQPQQQQAQPQAQQQQQQQHPQQQHQQHQHLPGKAAFPPGMGVPGMDHFHGTPYGMQAVP
MQPGHFEHLLNAMPVTGHSLSSSFATDNVHMSGAQPTLYLAAGENMAGSKPGVSAAGSAGGGGGGGSKTRLRWTPELHSSFVR
SVQQLGGPDKATPKGILKAMNMDGLTIFHIKSHLQKYRLNARVPGASSVDGGSDGCAAGDSAEGNSGSRPASAALDGLGSVPV
SALTRKNLEDALVLQMELQKKLHEQLELLLLLLLLLLLLLLMLLLLLLLLLLLLLLLLLLLLLLLLLLLLLLLLLLLLLRLLS
LEPRRSCDGSVACSCSCAEVFISGELY
>Entransia_fimbriata.PSR1
AQSSSKRMPADSGAEPTGSTGSGHHSQGRMSEGVYLSSNKQRLRWTPELHELFVSAVHELGGADRSTPKGILRLMGKQGLTIY
HVKSHLQKYRLAKLSGQSKYSQPATPPPQGADTGMAPLPPIRPGSSGGGAPTASVTSTVSEQDIEKDAGMGALPKSLQISEAL
RMQMEVQKRLHEQLEVQRQLQLRIEAQGKYLQQIIEEQQ
>Golenkinia_longispicula.PSR1
AGGSAKTRLRWTPELHSRFVASVNQLGGPDKATPKGILKLMGVEGLTIYHIKSHLQKYRLNIRLPEASSSGPLTSSDIGLDTP
DATMSISEATMPIVSEAQVQQRVEQSSVQTLTSLTSTQAETQSTTPTASASLEPVTHFVSQPMAVVDPTSESRRFTRKDLEEA
LLLQMELQKKLHEQLECQRQLQHHLEAHGRYIAQLMEQEGLAHRLADLTGQPLHPGPSSSEA
>Oedogonium_cardiacum.PSR1
ARAGTVKARLRWTPELHTREVASVQSLGGPEKATPKGILKLMGVDGLTIYHIKSHLQKFRLNMRLPESTNTSQGNEAGTSSKR
SKKDDLQGGDSPPFEQPKASETSTSSQPPPGALTTSTATSAPEALIHHEFPFPQFGSGSGTITRKDLEEAMLLQMEMQKKLHD
QLETQRHLQLSLEAHGRYIASLIEQEGLGQQMPELS
>Cylindrocapsa_geminella.PSR1
SGDYVYPAAAPGHLSQLGPGMGPGLQSMSHSMGFLAEGSHASGSHGMYPHSTIFTNTPSKDGSRKGRLRWTPELHGRFVSAVT
QLGGADKATPKGILKIMGVEGLTIYHIKSHLQKYRLNIKMPEAEAGGTGQSTDTALKVESNVPAVGQARPLGAAVPPGNGSFR
DMGAEVNPAAVSARADTTAGPFVGEQRGDQVTQPVRGQEQLPPVVQQSSAAPADSVNIHEVLKQHVALQRKLAEQLETQRQLQ
SHLEQHGRFLRELINTSASTS
>Scherffelia_dubia.PSR1
SPSAFKPRLRWTNELHNQFIEAVETLGGHGKATPSALLKHMNMEGLTLGHVKSHLQKYRTEIRRAKEARCKVKDVLKEIKRDK
ASKPGAGGKALDVAREAYEDGPNARELEEAMHTQLELQRLLCDQLEAQKKLQSSLEQHTKYISVLMRNKSDVRTKPRDPPDTG
SIEPGFQAVGAESGPSTSEA
>Hafniomonas_reticulata.PSR1
KSRLRWTPELHTRFVAAVSSLGGPEKATPKGVLKLMGVEGLTIYHIKSHLQKYRLNIKMPADGKQMSGSDMSGAVLGDPNRPE
LPSVSSLALDTSEATHLSPHLRPHGSPAPLAATSVPSLSSLPGSIDAKGSLEQALLFQMELQKKLHEQLEAQRQLQHSLEAHG
RYIASLMEAAQEGV
>Tetraselmis_chui.PSR1
QNTNFQMPSGMHFPNFNPNVPDGNMPNFGSSLFPPTTFRPRLRWTNDLHNQFLESVEQLGGHGKATPSAILKHMAVDGLSLSH
VKSHLQKYRTELKRAKAVRGRALNDMNQIKKGARRRAGEGSGGSSAEEGLDILGSTHEELQKQLAAKAKGPNAKELEDAMRTQ
LELQKMLCAQLEAQKKLQSSLEQHTKYISVLMQK
>Volvox_globator.PSR1
EFLPVLGFDAYSAKPTGLGLGGLLPDPPRTSTDGASTLLQSSDFMLSMPAVPHLMQPGVGTLQPPQSAFPDLTLPGAGSLGLN
SGLLHHPSGHFMGQPQRAATATAPGHGPAKSRLRWTPELHNRFVMAVNQLGGPEKATPKGILKLMGVDGLTIYHIKSHLQKYR
LNIRLPGDGVQGDSAADSDMSDGEPGGDGFGGPSTVAGEMQSGLAGGGGVSG
>Volvox_aureus-M1028.PSR1
GRAALPMDKAERAASNAIGNEDDWLLEFWPEPAAADELGPVAGAMQQQQQHPLQLDHSQLPEQVPHSGSFQMSQFGLSPPTSD
YLPGLQFDAYGSKPHGLSGLGGLFHDHQRSSTNGASTLLQPSDLLFPMCGVTHALMQHPAGVAGFQQPAFPDLPLGGVGLHPG
LLPGHYLSHQQRAASCPAKSRLRWTPELHNRFVASVNQLGGPEKATPKGIMKLMGVDGLTIYHIKSHLQKYRLNIRLPGETMP
GDSADTDGSDGEGEAPSASMDRLDRLEATQSGMLGGEGGAGGAGGGATTAATEQTVSISAQGKSGRRSGPAGGTSCSSGSAPS
ATRRNLEEALLFQMELQKKLHEQLETQRQLQLSLEAHGRYIASLMEQEGLTGKLPELTEAPLGGGGASASIGSRERRASGGLG
AGLSSVQQAPLGSAPPPLTTSKDRGGRGIAAGRAISGGCGALQSPATNLSGASPHLQASSGGVAGVGLQPLQPPPAAVGAAAG
RQGNQQQKPQHQFQNQQQQQQQQQKQVQAVGNSILTGVRHSPLHGLPSLGGSGGGGRGSVTSVTSSSSMHFQMQQDHQRLELM
RLGRLGSHPTPGSPSGNPLVDGGGGGGGAGVNEKPQHIVSNTGLAVVGESSIPLEQPVVILQDGGHSGQTAALAHQQPEPPQV
HPQPALRAASGQLESGLGLGDALEGIIGESGNGGGGANGGSVVPLPDFDFGDFPDLDSGGLEHQGLLGPGDL
>Ignatius_tetrasporus.PSR1
AKPRLRWTPELHKRFVHAVQQLGGPDKATPKAILKLMASPGLTIFHIKSHLQKFRLNIKLPDTKKEGSKATVSGQSEPFADQA
DNAVSMQFEEPTSEPEVAVSPSAGSSHVAYKGLLGKNLGEALVRQMELQKKLHKQLESQRHLQLSLEAHGRYIAGLIAR
>Gonium_pectorale.PSR1
MDKAERQAATSIGPEDDWLLEFWPEPTVSDLPRFGPAMQPLHQPHQPLDAPQHPELLSQQQQQHQALQLGAYGLQAQAPMGSD
YGLPGLGFDAFGGKAPLGMGGMLSEHPRASEGASAMLPPSDFMLPMGGVGSMPHLMQPGMGALQQHSFHDFSLGGAGLAQGML
HGHYMAHQQRAAAGPSKNRLRWTPELHTRFVTSVNQLGGPEKATPKGILKLMGVDGLTIYHIKSHLQKYRLNIRLPGEGGMQG
DSPVDSEMSEGEGAAPSA
>Planophila_terrestris.PSR1
QPSASTTGKSGRLRWTLELHKKFADAVAKLNGPDKATPKGILKLMDTKGLTIYHIKSHLQKYRSNMRTMQPAPMQAALSDAHT
ATHHDARSFGMQSFDAAVSASGAMHSGALTSNPLGLGLNFPQSSVSSMGAMLGGMGSAAAGPLTMTGLPPIRTDASLLRAASQ
GPSQGPDALSWAHAHGTSPAFARGGSPTCSVHSGAGRRDEGRGASGEGVDQSHSWTKALVKQMELQKQLHEQLSMQRQLQLNL
EAHGRYMLRLVAKEG
>Pteromonas_angulosa.PSR1
GGATASGGCRAGNGLGSGMSEPHEPRDGKQHLGSLDELWLDDHAVGELSWWPEPQLPLPDPLLLQGGAAGLMLGHQPQQQAQQ
QQPAYHGRMPPQQQQQQPSQFNNEYGYAPVPHDPYALGAHAQAPAGPSGDMSAGVSAVNYASLTPGPAIPQPHLPPQPMPQQL
QQHPGYMDPSMMGGMYGGQFYPAAPQQGPAKSRLRWTPELHNRFVAAVNQLGGAEKATPKGILKFMGVDGLTIFHIKSHLQKY
RLNIKMPGEGTPMPGDSDSESIDVGVRPMQQPQQQMQQPQQQMQMQQQGDTSMRSKSRGDSGDGVRGGGAVVAPASQAAGPGL
SSSTSSAINRKNLEEALLLQMELQKKLHEQLETQRQLQLSLEAHGRYIASLMEQEGLTQRIPQLHGNAQMPASTANAAAAVAA
AAAAAAAASSKHNDPRGAAAGSSASGQGGQSAAPSQPALEHRAASGAGAMPAHYQQQPPQQQYMQQQQQQHPVSDSGMAAAAA
AAAAAAAAAGMAGPSEMYAGHSQQQHHSSQQQQQPYLQLQHRTSQQQGEHQPSSIAAAGSAWGEAASTGHGPG
>Asteromonas_gracilis.PSR1
GPSKSRLRWTPELHNRFVQAVNYLGGPDRATPKGILKLVNGEGLTIYHIKSHLQKYRLNIKLPMDPSGSEYMSDSQQDVSASG
EMRSSVGHVVTRRRASPMPGAAQETPQQHATQVQAVGNTQEGTSRCLDTSAVASGDKSKSLEDALLFQMELQKKLHEQLESQR
QLQQSLEAHGRYIASLMEQEGL
>Haematococcus_pluvialis-B.PSR1
KSRLRWTPELHNRFVQSVTTLGGPDRATPKGILKLMGVDGLTMYHIKSHLQKYRLNIKMPAESGGQDSLSDSQDQQPPSAMEV
RSSSRGPTSTPQLRAPGSSYDCSGQAPALVSAASVTAVPAPSSAGAASSGTNRRNLEDALLFQMELQKKLHEQLESQRQLQLS
LEAHGRYIASLMEQEGLTQR
>Chlamydomonas_bilatus-B.PSR1
GHPGHHEQFRRQSEDKPGSSKSRLRWTPELHNRFVNAVNQLGGAEKATPKGVLKLVNVEGLTIYHIKSHLQKYRLSMKLPGDA
GGAESPAESDSGLEQGQGQQPTRRRSSM
>Vitreochlamys_sp.PSR1
GIGHNVLALQGLPSQSPAQMLPFVPDYSGQLGAGALPPGLHLQQGVLRSSSAAQAQRARLRWTPELHNRFVTAVNALGDKATP
KGILKLMGAEDLTIYHIKSHLQKYRLNIRLPSGPHADSGVSSDNDMGGMGNALSTVSSGPLGDLADFPQQHEFQTQQQRSLSM
SQTQQQLSQPQAPFIPGTASSPMQAASIAPNPGGSSPTMQQMMPDQQHRMGNLEHALLVQLELQKKLHEQLEAQRQIQMKIEA
HGRYLASLMASE
>Botryococcus_terribilis.PSR1
GMGGHQAPLGGAPIFTTSRSVSSSQGMRDPGKQRLRWTPELHARFVGAVNQLHGPERATPKGILKLMDIDGLTIYHIKSHLQK
YRLNIKLPGQGGPMFEEDDMSERRSTKRRRTKARSTKRRRKARGRISDSESEEDHYESEEEENDPEEVEAEEEEDDDGGEVAS
RGTGAPSGVPVPVPHHPARSQPMPTASSTEDSVDADGKKATRHDRQAALTKALNDQMEMQQKLMEQLESQRRLQSQVERHTAY
LRGLMEEEGL
>Eudorina_elegans.PSR1
GRVALPMAKAERASGTVVCNEDDWLVEFWPEPAAADLLSAVPGAMQAQQQHPHQQLDPSQLSELLPQQTGLQMGQFSLHQTNE
YLSSIQFDAFGGGKATGLAGLGGLLPDHQRSSTDGASALLQSSDFMLPMAGGLQQPAFPDLALGGVTLNPGMMPAHFLGHQQR
AASGPAKSRLRWTPELHNRFVASVNQLGGPEKATPKGILKLMSVDGLTIYHIKSHLQKYRLNIRLPGE
>Pandorina_morum.PSR1
GLGGLLPDHQRSSTDGASALLQSSDFMLPLGGVPHLMQPGVAGLQQSAFPDLALGGVGINQMLLQGHFLAHPQRAASGPAKSR
LRWTPELHNRFVASVNQLGGPDKATPKGILKLMGVDGLTIYHIKSHLQKYRLNIRLPGETTQGDSADSDASDGEAADPSASMD
RTVETQSGLGGGCGGSLA
>Oedogonium_foveolatum.PSR1
ARAGTVKARLRWTPELHTRFVASVQSLGGPDKATPKGILKLMGVEGLTIYHIKSHLQKFRLNMRLPESTSTSQGNEAGTSSKR
SKKDEPQGGDSPAVEQPKASETSTASQPPPAALTTSTATSAPAAALHHEFHFPQLGRGIGSITRKDLEEAMLLQMEMQKKLHE
QLETQRHLQLSLEAHGRYIASLIEQEGLAQQMPEL
>Chlamydomonas_sp.-M2762.PSR1
PQRPAPKGSSKSRLRWTPELHNRFVNSVNQLGGPDKATPKGILKLMSVDGLTIYHIKSHLQKYRLNVKTPGDSAAMYDMDSDG
DGEGEVTDTRPARSKGQSEATTSSGGTARGKHSNRQHQAASAPVGLPAAAPAPPVPGMTTAASLPVVSSNNRKNLEDALLVQM
DLQKKLHEQLENQRQLQAQLQAHGHYIASLMQQEGMATPAETQPPAPDTKPPGLPSTSAPAGLPGPLPP
>Chlamydomonas_sp.-M2762.PSR2/homologue
GVSKSRLRWTPELHNRFAAAVRLLGGPDKATPKGILSQMSAPGLTIYHIKSHLQKYRLSSKSPGNFSLNDDSDDGLAGEGDED
TSCMASGHRQDFAAAALPGDADRRAAHPGSPRRAIVTNMEVSGSPAPSVRPQAAVMTAPRLPDAAASNRRNLEMALLRQMELQ
KKLHEQLEAQRHLQLSLEAHGHYIATLMQKEGYAGGPTPPEPAAGACPAPATAAAGTAVISSAVPQGLARACSS
>Chlamydomonas_noctigama.PSR1
YQMPGIVGAAPTKKGRLRWTPELHACFVNSVHQLGGFEKATPKEILRLMKTEGITLYHIKSHLQKYRHCMKLGRLGGTDSSDA
SENLPGDQQSPQPILDCHMPGRTDGSLEVAPSRPADGGRTTCHRHNDSTRQFSDANVQASASSCSIRRTALEEAIALQKELQK
KFREQMQTQIELQARLEAHGRYIATLVER
>Carteria_crucifera.PSR1
GTPKSRLRWTPELHNRFVNAVNQLGGPEKATPKGIMKLMSVDGLTIYHIKSHLQKYRLNIRLPAESQLTDSSTENKHELQGQS
PVQEPQQQERDCGGTSAIPCELTVPTTTSGSGAVTTVPNALYTNVQASIVASQASIVASAHPPPSTAEPPVQAGPSSSERRSS
PEPSSSTRKNLEEALLFQMELQKKLHEQLESQRQLQLSLEAHGRYIASLMEQEGLTHKLPELTGQTLGAPSS
>Volvox_aureus-M2242.PSR1
GRAALPMDKAERAASNAIGNEDDWLLEFWPEPAAADFLGPVAGAMQQQHPLQLDHSQLPEQVPHSGSFQMGQFGLSPPTSDYL
PGLQFDAYGSKPHGLSGLGGLFHDHQRSSTNGASTLLQPSDLLFPMCGVTHALLQHPGGVAGFQQPAFPDLPLGGVGLHPGLL
PGHYLSHQQRAASCPAKSRLRWTPELHNRFVASVNQLGGPEKATPKGIMKLMGVDGLTIYHIKSHLQKYRLNIRLPGETMPGD
SADTDASDGEGEAPSASMDRLDRLEATQSGMLGGEGGGGGTGGGATTAATEQTVSISAQGKSGRRSGPAGGTSCSSGSAPSAT
RRNLEEALLFQMELQKKLHEQLETQRQLQLSLEAHGRYIASLMEQEGLTGKLPELTEAPLGGGGASASIGSRERRASGGLGAG
LSSVPQPPLGSGPPLLTTCKDRGGRGIAAGRAASGSCGALQSPATNLSGASPHLQASSGGVAGVGLQPLQPPPAAVGAAAAAS
QSATEASASVPEQQQQQQQQQQQQQQQQQQKQQQQQNQVQAVGNCILTGVRHSPLHGLPSLGGSGGGGRGSVTSVTSSSSMHF
QMQQDHQRLELMRLGRLGSHPTPGSPSGNPQVDGGGGGAGVSEKPQQVVFNPGLVVVRESSIPLEQPVVILQDGGHHGQTAAL
AHLQPEPRQVHPQPALRSASGQLGSGLGLGDALEGIMGESGNGGGGANGGIVGPLPDFDFGDFPDLDSGGLEHQGLLGPGDL
>Phacotus_lenticularis.PSR1
PKSRLRWTPELHNRFVSAVNQLGGADKATPKGILKLMGVDGLTIFHIKSHLQKYRLNIKMPGDSSMLAGDSDSESIDPQRSLR
MPEPMRSKSKGDSGDAQRGPAVPSGAPAPAGPSMPAPSPSGAGPSMPAPSSSTSSAINRKNLEEALLFQMELQKKLHEQLETQ
RQLQLSLEAHGRYIASLMEQ
>Stephanosphaera_pluvialis.PSR1
GQALM```QPQFSSQAPKPDPAAPKQRLRWTPELHNLFVQAVDQLGGPERATPKGILNLMSVEKLTIYHIKSHLQKYRLNIKCPNG
DSGAAGDSDSYDQAPSGGVVEGRSLSRGSVPTLTHPHASLTSLLTGSMPSAAATSPQAVTPAASAIHVHSPPPVGCPTQPSLQ
LTVGTSAPGSLSQAPVSQLSNMSGGLSTTVTAANRKNLEDALMVQMELQKRLHEQLEQQRQLQLSLEAHGRYIASLMEREGMT
>Chlamydomonas_eustigma.PSR1
MADPIGQPSLPTDDPLLLTLKTGGVDEPDLDENSWLEFWPESELPSMHSFLPQANNVPIDESYRQGFALQSAIPDIPRMQGGL
LDNYDRVPTLLSAGSARDMHMLADFHGNMKSLPHHAGFSSFSGDMGPSAGSYYHEKESRHGSNSRSRLRWTPELHNRFVNSVN
QLAGPEKATPKGILKLMNVEGLTIYHIKSHLQKYRLNIKMPGDMNLESCGDDSDMDERPTSTTPVDRNRRAPDLERQVSLNMD
RLGRAGKVASERETLDQGRRGMDTSSGLAGPATGHTESTPPRPSTSAGTTAPLSSSSSALNRKNLEDALLFQMELQKKLHEQL
ETQRQLQLSLEAHGRYIASLMEQEVLASKQDGQQPSTEPSLGGGGGTTRGGITAAGLCRPPSGASDEVMIADKAGMVSTSGAA
SPQYLDHGGAKQGGAHLQYFSSAGSEASASAAGTGLHGLSVSASNGAHGAMLSGNQFMGMGGDSSMALMSPGKHMETHMGGTV
MHGKQLHGVMRGGMVGTAAAPSPPLLPLMDAHPSHDGSPGASLLPTSLMMVQQSSPDLELLVHEAGSLASAGDCNHASKRIKL
ENEL
>Chlamydomonas_incerta.PSR1
MDKAERAPGGPNAASEDDWLLEFWPEPAADFPAPVAAMQPQHQDATQLQEAVPQQQGLALGAYGLAQQPSDFMQSSMPGFDAF
GSGKAATLAGLPGLLPDAQRASTDGASALMNAAQQSSEYMLAAGMGGVQHLLAPSVGTALPGSGHTGFADLSMGGLAGGLAGL
GGPGMMHHGQFFMQPQRAATGPAKSRLRWTPELHNRFVNSVNSLGGPDKATPKGILKLMGVDGLTIYHIKSHLQKYRLNIRLP
GESGVAGDSADGSDGERSDGEGGGGGGRRATSLERADTMSGMAGAVAGGRPGGALLSPGLAGATTSTGAVGGGGGLMTEPSIS
RGAVLNAAGAAPAGVVAAAVGGSAGVKRPAGTSLSSGSTASATRRNLEEALLFQMELQKKLHDQLETQRQLQLSLEAHGRYIA
SLMEQEGLTSRLPELSGGGPAAPPAGAGGAAGGMIAPPPAQQQLQHQPQQLLQPQGSLPAGGGSGVDAGSGGGGMNLQPQHQH
VHLHHHQQQLQPLARHCDTCGAGGAGGAPSGGSSMQQLQAAEQQRTELVATGRLGSMPAPASSSPLAGQQHQPLAGGAAHVHM
HAHTPGAQPQPHVQRQDSYAGAAAAAAAAAAAALPQSHSHTLPADLSSNAVADPSAGPIKPEQGLSPQQQQQQQEQQEAEQLA
QGLLHDSSAGAGAVSGSDGGGLGDFDFGDFGDLDGGAQGGLLGAGDLIGIAELEAAAAHEQQLQQQQQQQQQQQQQQQQLQGQ
EQEQLDADRAKRQRLEQ
>Chlamydomonas_schloesseri.PSR1
MDKAERAAGCTNAASEDDWLLEFWPEPATDFPAPGAAMQPAHQDATQLPETIPQQQGLALGAYGLTQQPADFMQSGIPGFDAF
STGKAPGLAGLPSLLPDPQRASTDGASALMTAAQQPSEYMLPPAMGSVPHLLAPSVGTVLPGTGHTGFPDLSMGGMPGGLAGL
GGPGMMHGQFFMQPQRAATGPAKSRLRWTPELHNRFVTAVNQLGGPDKATPKGILKLMGVDGLTIYHIKSHLQKYRLNIRLPG
ESGLAGDSADGSDGERSDGEGGGGGGAMGRRASSLERADTASGMAGPGAVAPGRAGSTPGGQLLSPGVGATAGAMAAAAEPSM
SRGAVLGASGAAAAAAPAAAGAAAGGVKRPAGGPSLSSGSTASAARRNLEEALLFQMELQKKLHEQLETQRQLQLSLEAHGRY
IASLMEQEGLTSRLPELSGGAPAAPPAAPGGMLAPPPQPQQQQQQQQLLQPQGSLPAGGTTAGLDAAAATGCGGGEGPQVQQQ
QQQQHHVRHCETCGAGGAPSGGSSMQQLQAAEQQRNELAAAGRLGSMPAAASSSPHAGHAPLQPPGAHLHVPTPGMHQQQQQH
IQPQDSYAGAAAAAAAQASPAALPQSHSHTLPGDMSSSGVMEQPGPGLVKAEPGLSPQQQQQQPQMQQDADQGLLGDGGAGAA
GVSGSDGGGLGDFDFGDFGDLDGSAQGGLLGPGDLIGIAELEAAAAHDQQQQLQQLHQQEEQEQLDADRVKRQRLEQ
>Chromochloris_zofingiensis.PSR1
MDQRREPAAASRDADTADINWLEFWPESEFKIDGAPAGGSMDPSLGTLGGLGDYLGSNLQHPQLATAAPLQLTLPGEFGSAQG
LPLLSSLEAYQGSGDLNVLQSTQPGQLPQLLSSAPLAGHSLSSSYGSDPSGFTANLTSPALYPTGSYLAQNSKPGLPPKTRLR
WTPELHSRFVSAVHQLGGPDKATPKGILKLIGVDGLTIFHIKSHLQKYRLNIRLPESGRSDSQGGSEPLEGGSGADSRMRAPS
STQTQAKLGLSNQLESGQAGTEATPSATAASSQDYAAAHTVSRPRSSNAGPSSGAGKAAVGDQLPAAGGSGSYSTVGQGGAKP
AAQKQQQLKQQLTAGLPISAVTRKDLEDALLLQMELQKKLHEQLESQRQLQLSLEAHGRYIASLMEQEGLGHRVPDIAQLTGV
QPEHLQHKEQQAGGVPLSFTQSLTEDLNVDDSALHMFPGGHSAGPSHRHQHATEQGLPDSPHLLLNFPELNELADVPQAGAGS
NMLLPHMPMGQQNHMQPTHKRQRLDEGGSAQNHGSHSGSRQQH
>Coccomyxa_subellipsoidea.PSR1
MQQNQHYNWVSQHVSVEDHQEPHTQHQLPQTVGHEHLTAAHAAFSDFGQHRGDQAIAGITGDQAELLEAISGTETELGLPYTA
LDHLQPHHPQNDLHQHGVGYGMERNPSEPTQSQGKDAKPRLRWTPELHARFVSAVASLEGPDKATPKSILKLMAVEGLTIYHI
KSHLQKYRLNVRLPGESGDMISGPDESEEPSRRKRRSRSHGQASSRRRSSRQRKRRRSSDEDSDEDDMEDEDMDDDENFEEGI
SRARPGTSVSGINGSSPHGGSPRGGSPRGVSPRGRSPRGGSPHGGGNSMACLQVEPLEVPDLDPEKQHSLEEALLKQMDMQKR
LHEQLEEQRRLQLSLEAHGRYITSLIQKKGLEGLPPQTKEALDAALVPPQGSGLSTLTHNTAPQWTPSVSEGSSLAQQVGHVM
HHSTAFMLGSASATDPESSLLLDTNMQAAAAVWDPSQAHGLDQSGSKQLYEEPKELYEEPGQLYEERGGHVKPEEQL
>Symbiochloris_reticulata.PSR1
MDTHDIAPALPERSLEWLEFWPEAEFRAEDHSMNSNPFCWVQHDDQQPSSGAKASAGPVSAAQTSEPGMLFPGQLQPVSAALL
SHFTEVHTGVQADVSHSFLPPDYMQPHFCGSDLPEAAHSMPLASAAQQAPVFGATANEPSSAGAGSSQAGKPRLRWTPELHSR
FVAAVNHLGGPDRATPKGVLKLMLVEGLTIYHIKSHLQKYRLNIRLPGDSGPVGSLSGSRKKRKRSRRARSSDLEDEEEEDDM
DEADSMEDMLPGDELHGRQQAVGEAGLALDAALPEQGNAQVPGQQPEQQPNAQRQRDLEEALILQMDMQKRLHEQLESQRQLQ
LSLEAHGRYISSLIEREGLQSKLPAGTHAAMQSGLPRLPEASLGMAAGMCGPADGSGAGTIAPGTSGGMSWGQMTHVTLPHSA
ESPPLLSHTSRTGATAADAGQFLMVGDPGDLGPLPSMLLDTDLQAAAAVWDDGMHRPRKHAPNGHLEHASGLDEGLFDQHEGE
EHGRLQRRRQPSSRLRQS
>Edaphochlamys_debaryana.PSR1
MAKAEGRPGTIVGSEEDWLLEFWPEPTLELSGPAAMQPQQSPSLDAPITDLQQLAPQQTSQQAQQPGGLPLGQYALASAADYL
QTAQHALSAYDPYRTKSAPPLPLGLLPDRPSDCASGLLPPPAGGEYLGALGTAQGGLGPVPHPLMAPGAVSGLQQGQPGGGYG
DLGLGAMGMGMGGLGLQQGMLHPHAHYFAAPPRAAAGPSKSRLRWTPELHNRFVQAVNTLGGPDKATPKGILKLMGVDGLTIY
HIKSHLQKYRLNIRLPGDSAAGPQGDSADDSDAEGGGGGTTATGMAAAPSMSLDRGGMETTSGLLGRRLGSNAATAAAAAGFL
AGGGGGGGGGMAEPSLSNSIAAAQVAQQQAAAAAAAQMAAARPAGGSTSSGSTPSATRRNLEEALLFQMELQKKLHEQLETQR
QLQLSLEAHGRYIASLMEQEGLTSRLPQLSSGDGPTAQLALPGPGGEGGGDGLQRQPSGIGGGGGGPQQGGPLVGATGQGVDH
AGLGGVGPDGRRISSQGLGAPSPQALLPFQLSSAGQPTGRHQLGMQPSPQHLPGPGGDGGGGGGPGDEHQRRRSEIAYDGTGG
SGLTGGASGGSSVQQLAVAEAQRHDLMRAGRLGSMPSAAAAALQAAGSNSLPQQHMYSPAQQDSLGLSQQQQQQQAQADAQAH
AQAHAAAQEHAAAAAVAAGMQLSMAHAPSGSGLGDGGGGLGDGGGGLGDFDLADFVGDLDASGVAALEGQGFAGLQGGLQGDS
EMGLLAGIGDDLAAAAAEAQAQGLVSPRRGSSGGEDSGRSKRARLQGSSSGEGQG
>Enallax_costatus.PSR1
MDPGPNHSLGPLEPDHCDLGYLEFWQESEFKLEPAAHHTLGMDHLGDHFLAAGAVGGSAGQYGQLGLAAGPDPYSNQGIPLVP
ALNDQHFQAGDVSLLSTSTGQGNQVPQLLTTPALESYTSSYGADPLSSMPSGAMLYSSGAFAMPGSKGSSAFDAPSNKTRLRW
TPELHSRFVSAVNQLGGPDKATPKGILKLMGVDGLTIFHIKSHLQKYRLNIRLPEGAQPAMSTGSMQEGDAAAAAVDSAADTQ
TAVMSGAQAAAAQQPSQQQQQRGQQDKSGQQDKPTQQQQQQAPALVPQPSSSAGRAAASLSPLIREGSTTSIPGLSSGAVPDM
QAPLLPPGTGSGGPAGQQQQQQSQQQPPPPQQQLKQAQQQPLQQPQQHARPVPETAAAAGGAAATDENNDAAIKHSTRRDLER
ALLRQMELQKQLHEQLEMQRALQHSFEVHQRYIHSLMEQEGLAHKIPEMSAALGAVAAATATAPPGSVVSEAMPVQPAQPSNN
SQPLQQQQQQQPPGAQAGAAVPAPGQQQQQQVLQLPQKQQHTGHASANDAAPVAAAAVTDQFLSDAELLMGFPDLQHDTGDID
PIQQHLLGDEAAGGPPKRQRMSGQDV
>Mesostigma_viride.PSR1
MNRPPVGSASRTADQQLSSSAEVQPRTVQKLPTTVDELINQEWPIWGELAPNDDSITTCWTDLLTGPPPKNQDMHRPQHATIQ
DDTSPGLYLARQQYLPGMGTLPPGGVPPLCAPPGLMDGGGMNLVPGMQASMAAAQSQQPPKQRLRWTPELHDRFVNAVQNLGG
ADRATPKGVLRVMGVQGLTIYHVKSHLQKYRLAKFLPEEGGNSSKSLGGSKRDTDSDNDDASDGDPLKMADLKAGATELLTGE
DGSVNIEEALRMQMEVQKRLHEQLELQRALQLKIEAQGRYLQQIMEEQRNAALARRAQAGGAASGATTQGQATSAGQAASASS
SRSSAGGGGKGPEGAAAPAAGEGGAGADSISHAADGARAGAEAGDAQRQAVASPSGAPVFAASGVHDADGAGATCPAVGAAGG
HAPSPALVPKTESVACGGSLAMPDALASLPGGGGHHLGASGKLPGCELPLPSWSEPGAALLTANGGILPFPSKVEGRNLPQLS
LPSHLLLGVEDVDDGGGGGGGGGGGGQSLQSGVGVGSKRAYDEMMGGGIAMEDGAGDRLHTDGSGLPTGGSLLPDDASLLAAQ
GGHASGGADPAPHML
>Raphidocelis_subcapitata.PSR1
MAERTPGSPAGEGDEAVLAGLAGWLNDELSYWPEWPVGPPAPPLDPQAHCDGPVIALPGAHCPMEQRQVAAGPPGPHGGAGPH
AVAQPQQQHPALQAGQGHALDAFQSYQATAYGMQLAVHAQQGGFDPGMLGAAGALAPGALFGVPPAYGMAGGKPGAMAGGNKS
RLRWTPELHASFVAAAESLGGADKATPKGILKLMAVPGLTIFHIKSHLQKYRLNVRAPDGTEGASDGGGESAVEGASGEGGAT
VRMGALRAESLDATAPSSALALPPTALGASPAVGVKPEHPEVDAHSLLKQQQHAVPASTTSTCAGLSSATGLEAAAAAGGAGS
EAAAGGPSTARRRNLEDALQLQMDLQRRLHDQLEAQRALQLSLEAHGRYIARLMEQEGLGHRLQDLAAITAPGPGAGAEAEAA
PGGGDGGGAAGSGGAGSGGAGPGGAPAAAPASEANSSGLRAAAGGCGGGRSVAGGCCDGALPLARAGSSALDSSDHPAEPHQQ
PARWQQPTPPPSASGRRDDRSQDQRLHAAAGQLLAWGRSAPPPHDAAGLDAAGAPQGKRPRLSGA
>Symbiochloris_reticulata_Africa.PSR1
MLFPGQLQPVSAALLSHFTEADVSHSFLPPDYMQPHFCGSDLPEAAHSMPLASAAQQAPVFGATANEPSSAGAGSSQAGKPRL
RWTPELHSRFVAAVNHLGGPDRATPKGVLKLMLVEGLTIYHIKSHLQKYRLNIRLPGDSGPVGSLSGSRKKRKRSRRARSSDL
EDEEEEDDMDEADSMEDMLPGDELHGRQQAVGEAGLALDAALPEQGNAQVPGQQPEQQPNAQRQRDLEEALILQMDMQKRLHE
QLESQRQLQLSLEAHGRYISSLIEREGLQSKLPAGTHAAMQSGLPRLPEASLGMAAGMCGPADGSGAGTIAPGTSGGMSWGQM
THVTLPHSAESPPLLSHTSRTGATAADAGQFLMVGDPGDLGPLPSMLLDTDLQAAAAVWDDGMHRPRKHAPNGHLEHASGLDE
GLFDQHEGEEHGRLQRRRQPSSRLRQS
>Tetradesmus_deserticola.PSR1
MDSGAHDLGDHTGDWLEFWHESEFKLDGVSTAAAQPGQHAPMDLPGGLGDFFLPSGSMLPQPHSGDAQQLVLAPAGDPYAGSL
TMLPGLEQQQQHYKGPDLSFMSTSSGAAGQMTQLMPPTAQLESYTSSFSSDPTLSGMHSAPMLYHAASFQLPGTRSGSLQEAP
AGKTRLRWTPELHSRFVQSVNSLGGPDKATPKGILKLMSVDGLTIFHIKSHLQKYRLNIRLPETSEMGAQPANSSGSPDQEAT
AATDSAADTHATLATSTINPSAAAAVAAGAAAPTAAVAPASASAGGGSSLQQQQQQQSLVPTSQQQQQQQPPPPQQQQQQRIL
SGVEQLSGASPLQLTTSGVLEMPDSAASAAQQQQQQQQQQQQQPTGAAADAAEDSLHMKSDTRRDLERALLQQMHLQKKLHEQ
LETQRQLQHSLEVHQRYIHKLMEQEGLAHKIPEMSAAFNAGALPPPGSVVSEAMPGQPLAVGTAPQQQQQQQQQASSAAPPLQ
RHHSLPHQQQLHTGVGNSDAAAGVGTSKRSSSHHHHHHHQHHQQHHPQQQHHPQPMQQQQEPGQDAAGIDPLPGSCGNLLSDH
ELLLGFPELRDSGDEGGGMGLLSEPGQPQGKRQRLLTPDIAKWPSVDSAEGQH
>Tetraselmis_striata.PSR1
MNIRHDDDAAAASVQVRERVPWSQGIRGVEGAVVVVFILARFTPTAPQTRQTLQPPYRQLARHREDPATSPDPRTGRPAMDLN
EDADAELNFFKAMEAFSPPSFEGGEAEDHLHGLSVPGLPHMAGATDTHAHNPPASTGDGSQSATVHNTSGARGHDLLVNNGHS
MWEPLSFEEVMRNGGVNPSQASSLASTSTAATELLMHRGNTFLPSGNGGGRQAPPGQFGMGGMPSMMAFGAPQQQQQHQQHQP
TPQQQPPQRNGSEDGMQHFGGLFPQSAAFRPRLRWTNDLHNQFLDSVERLGGTDKATPSAILKHMGVDGLSLGHVKSHLQKYR
TELKRAKAVRGKAMDDMHQMKKGARSKAAAADVAAEAAEVVAEASGSAEAGLEQLGATQRELQRQLAARAASGPNAKELEEAM
RTQLELQKMLCAQLEAQKELQRSLEQHTKYISVLMKRQSGDDLHAHGEGDTAGEHEMSKA
>Trebouxia_sp..PSR1
MDNDTIDWLDLDYWPEKDSKKPADMDNSFAWLAQQAQPLSGQPLPGSQYQVQPHVMQPHDGLLYHDTFHPHSTAGSLLSDLSG
DLLDTTAVDISNAQFEAIPSQSPHQQSNMQLRSDTAHNGAPQPLQDMIQAPVFGRSTSSMSQQAGNNSQSAAAQAAGKPRLRW
TPELHTRFVGCVSQLGGPEKATPKGIMKLMSVEGLTIYHIKSHLQKYRLNIRLPESEQVEMSEAVSGEHEGRKSQRGKRRSTR
KQRKRSKRSSSRRRALEKSDGDDDEADDLDDDQFDEEEGDNELDGHAASSGVGEASSMLDGVTNREEDAQREVQRQRNLEQAL
LIQMEMQKKLHEQLESQRQLQLSLEAHGRYITSLIEREGLQHRLLPQLVAAAAPSLARTVPALAALAASMPPGSSGQISDQQT
HYMPLSASGASEFSPQQLLAGRESSLPNSVNLNQDPSPGATDAARSLDVSPSSLSRHVSGAVPRNPFGTINQAAFGEPSSPGL
LLNTDLQAAAAAWDDQQRHILTGPGSRPLDGMPAVPGQ
>Chlamydomonas_reinhardtii.PSR1
ATGGACAAAGCTGAACGCGCTGCTGGTGGCCCTAACGCTGCAAGCGAGGACGACTGGCTGCTGGAGTTTTGGCCGGAGCCTGC
AGCGGACTTTCCTGCACCGGTCGCTCCGATGCTGTCGCAGCATCAAGACGCAGCACAGCTGCCTGAGGCCATGCCGCAGCAGC
AAGGACTGGCGCTGGGTGGATATGGTCTCACGCAGCAGCCTTCTGACTTTATGCAAACGGGCATGCCCGGCTTCGACGCGTTC
AGCAGCGGGAAGGCTGCAACCCTCGGGCTGCCCCTGCTTGCCGACCCCCAGCGCGCCTCCACCGACGGCGCCTCTGCGCTTAT
GAACGCGGCGCAGCAGTCCTCAGAGTACATGCTGGCCCCCGGCATGGGCGGCATGCCGCATCTACTAGCACCGAGCGTTGGCA
CGGCGCTGCCCGGCACTGGGCACACCGGCTTCGCGGACCTGTCCATGGGGGGCATGGCGGGGGGCATCCCGGGCCTCGGGGGG
CCAGGCATTATGCATGGGCAGTACTTCATGCAGCCGCAGCGAGCAGCCACGGGCCCCGCCAAGAGCCGGCTGCGCTGGACGCC
GGAGCTGCACAACCGCTTCGTCAACGCGGTGAACTCGCTGGGGGGGCCGGACAAGGCCACGCCCAAGGGCATCCTTAAGCTCA
TGGGCGTCGACGGCCTCACCATCTACCACATCAAGTCGCACCTGCAGAAGTACCGCCTCAACATCCGGCTGCCGGGAGAGAGC
GGCCTCGCGGGCGACTCGGCGGACGGCTCGGACGGCGAGCGCTCGGACGGCGAGGGCGGCGTGCGGCGCGCCACCTCGCTGGA
GCGGGCAGACACCATGTCGGGGATGGCGGGAGGGGCCGCCGCAGCGTTAGGGAGAGCGGGCGGGACGCCGGGCGGTGCGCTAA
TCTCCCCCGGCCTTGCCGGCGGGACGTCAAGCACCGGTGGGATGGCAGCCGGCGGCGGCGGGGGTGGCGGCTTGGTGACTGAG
CCCAGCATCTCTAGGGGCACGGTCCTCAACGCGGCCGGCGCAGTTGCCACCGCCGCGCCGGCTGCGGCGGCGCCTGCCGGCGG
GTCCGCCGCCGTGAAGCGGCCGGCGGGTACGTCTCTGAGCAGCGGCAGCACTGCCTCGGCTACTCGGCGCAATCTGGAGGAGG
CGCTGCTGTTCCAAATGGAGCTGCAGAAGAAGCTGCACGAGCAGCTGGAGACGCAGCGTCAACTGCAGCTGAGCTTGGAGGCG
CACGGGCGCTACATCGCCAGCCTCATGGAGCAGGAGGGACTCACCTCGCGACTGCCCGAGCTCAGCGGCGGCGCGCCGGCGGC
GGCGCCTGTGGCCGCAGGCGGCGCAGCGGGCGGCATGATTGCGCCGCCGCCACCGCAGCAGCAGCTGCAGCACCAGCCGCAGC
TGCTGCAGCCGCAGGGCAGCTTGCCAGCCGGCGGTTCCTCTGAAGCCCATGCCGCAGCCGGCGCCGGCACGATGGTGGTGCAC
CAGCAGCAGCAGCAGCACGTGCACCATCATCACCAGCAGCAGCAGGTGCAGATGCAGCAGCATGCCCGCCACTGCGACACGTG
TGGCGCCGGTGGCGCTGGGGGTGCGCCCAGCGGCGGCAGCAGCATGCAGCAGCTTCAGGCTGCGGAGCAGCAGCGCACGGAGC
TTGTTGTGGCGGGGCGGCTAGGCTCCATGCCGGCGCCCGCCTCTTCGTCGCCGCTAGCAGGGCAGGCACACCAGCAGCAGCCG
CTGGCCGGCGGGGCGGCGCACTTGGTGCACGTGCACTCGCACACGCCTGGGGGGCAGCCGCACGTGCAGCACCAGGACGCGTT
TGCCGGCGCGGCTACGGCGGCAGCGCACGCTTCGCCGGGGCTGCCGCAGTCACATTCGCACCTGCTCCCAGCCGACCTCTCCA
GCAACGCCGGTCCTGACACAAGCGCGGGGCAGATTAAGCCTGAGCCTGATATGTCGCAGCAACAGCAGCAACAAGAGCAACAG
GAGGCGGAGCAGCTTGCGCAGGGTTTGCTCAATGACAGCAGCGCTGGCGCGGGGGCTGTCAGCGGCAGCGATGGTGGGGGCCT
TGGGGACTTTGACTTCGGTGATTTCGGGGACCTGGACGGGGGAGCCCAGGGCGGCCTACTAGGCCCCGGAGACCTCATTGGCA
TCGCCGAGCTGGAGGCAGCGGCCGCGCACGAGCAGCAGCAAGAGCAAGAGCACGACCCACTAGATGCGGATCGCGCAAAGCGG
CAGCGAGTGGAGCCATAG
>Monoraphidium_neglectum.PSR1
ATGCAGCAGGACCTGCTGGGTGGCCCTCCTGGGCCCATGCATCATGATGAACAGCACGAAATGTTGCAGCAGCAGCCGCAGCA
GCAGCAGGCACAACCCCAGGCGCAGCAGCAGCAACAGCAGCAGCACCCGCAGCAGCAGCACCAGCAGCACCAGCACCTGCCCG
GCAAGGCCGCCTTTCCCCCGGGCATGGGTGTGCCTGGCATGGATCACTTTCACGGCACACCCTACGGCATGCAAGCTGTGCCC
ATGCAGCCAGGCCACTTTGAACATCTGCTCAACGCAATGCCCGTGACGGGTCACTCGTTGAGCTCGTCGTTCGCAACGGACAA
CGTGCACATGAGCGGAGCCCAGCCCACGCTCTATCTGGCCGCGGGTTTCAACATGGCCGGTTCCAAGCCAGGCGTGAGTGCGG
CTGGCTCGGCTGGCGGCGGTGGCGGTGGCGGCAGCAAGACGCGCCTGCGATGGACGCCCGAGCTCCACTCGTCATTTGTGCGC
TCCGTGCAGCAGCTGGGAGGGCCCGACAAGGCGACGCCCAAGGGCATCCTGAAAGCCATGAACATGGATGGCCTCACCATTTT
CCACATCAAGAGCCACCTGCAGAAGTACCGCCTGAATGCGCGCGTGCCCGGCGCCAGCAGCGTTGACGGTGGCAGCGATGGCT
GCGCGGCTGGGGACTCGGCAGAGGGCAACAGCGGCAGCCGACCGGCCTCTGCAGCTTTAGACGGGCTGGGCAGCGTCCCCGTG
TCTGCCCTGACGCGCAAGAATCTGGAGGACGCCTTGGTGCTGCAGATGGAGCTCCAGAAAAAGCTCCACGAGCAGCTCGAGCT
CCTGCTGCTGCTGCTGCTGCTGCTGCTGCTGCTGCTGCTTATGCTGCTGCTGCTGCTGCTGCTGCTGCTGCTGCTGCTGCTGC
TGCTGCTGCTGCTGCTGCTGCTGCTGCTGCTGCTGCTGCTGCTGCTGCTGCTGCTGCTGCTGCTGCTGCTGCGGCTGCTGAGC
CTGGAGCCGCGTCGTTCCTGCGACGGCAGCGTCGCGTGCAGCTGCAGCTGCGCTGAGGTGTTCATCTCGGGTGAGCTCTACTA
G
>Entransia_fimbriata.PSR1
GCCCAGAGCAGCAGCAAGAGGATGCCGGCTGACTCGGGGGCAGAGCCTACTGGGAGCACCGGCAGCGGCCACCATTCGCAGGG
GCGGATGTCCGAAGGGGTGTACCTTTCCAGCAACAAACAGCGCCTCCGCTGGACGCCGGAGCTGCACGAGCTTTTCGTCTCTG
CTGTGCACGAGCTAGGAGGCGCAGACCGCTCGACCCCGAAGGGCATCCTCCGCCTCATGGGCAAGCAAGGCCTTACCATCTAC
CATGTGAAGAGCCACCTCCAGAAGTACCGTCTTGCCAAGCTCAGCGGACAGTCCAAGTACAGCCAGCCAGCCACCCCGCCCCC
TCAAGGCGCCGATACTGGGATGGCCCCCCTGCCACCCATACGCCCCGGGTCCTCCGGTGGTGGCGCGCCGACAGCCTCCGTCA
CCTCCACTGTCTCGGAGCAGGACATTGAGAAGGATGCGGGCATGGGAGCTCTGCCTAAATCACTGCAGATCTCAGAGGCACTC
CGGATGCAGATGGAGGTCCAGAAACGCCTCCATGAGCAGCTCGAGGTCCAGCGCCAGCTCCAGCTTCGTATTGAGGCTCAGGG
GAAGTACCTACAGCAGATTATAGAGGAGCAGCAG
>Golenkinia_longispicula.PSR1
GCAGGTGGATCGGCTAAGACTCGACTGCGTTGGACACCGGAATTGCACAGCAGATTTGTGGCCTCGGTCAACCAGCTTGGGGG
ACCAGACAAGGCCACTCCTAAAGGGATCCTGAAACTGATGGGAGTTGAGGGGCTCACAATCTACCATATTAAGAGCCACTTGC
AAAAGTACCGCCTTAATATCCGTCTCCCCGAGGCATCGTCCAGTGGCCCCCTGACCTCCTCCGATATCGGTCTAGACACCCCG
GACGCGACGATGTCAATATCAGAAGCAACCATGCCCATAGTGTCTGAAGCTCAGGTGCAGCAGCGGGTGGAGCAGAGCTCTGT
ACAGACCCTAACATCATTGACGAGCACCCAGGCTGAGACGCAATCTACAACTCCCACAGCTTCTGCTTCACTAGAGCCCGTCA
CCCACTTTGTCTCCCAGCCCATGGCAGTGGTTGACCCCACCTCAGAATCCAGGAGGTTCACCCGCAAGGACCTGGAGGAGGCA
TTGCTGCTGCAAATGGAGTTGCAAAAGAAGTTGCATGAACAGCTAGAGTGTCAGCGCCAGTTGCAACATCATTTGGAGGCACA
TGGGCGCTATATCGCTCAGCTCATGGAGCAAGAGGGCCTGGCCCATCGCCTGGCGGATTTAACAGGCCAACCCCTGCATCCGG
GACCCAGTAGCTCAGAGGCA
>Oedogonium_cardiacum.PSR1
GCCCGTGCAGGCACTGTGAAGGCAAGGCTGCGCTGGACTCCTGAGCTTCACACACGCTTCGTGGCATCTGTTCAAAGTTTAGG
CGGGCCAGAAAAAGCAACTCCCAAGGGCATTTTGAAGCTGATGGGAGTAGACGGGCTAACCATTTATCATATAAAGAGCCATT
TGCAAAAGTTTCGTCTTAACATGCGTCTGCCTGAATCAACTAATACTAGTCAAGGAAACGAGGCTGGCACAAGCAGCAAACGA
AGTAAGAAAGATGATCTCCAAGGAGGTGATTCCCCTCCTTTTGAGCAACCGAAAGCCAGTGAAACCTCTACATCTTCACAACC
ACCACCAGGTGCATTGACCACCTCCACAGCAACCAGCGCACCTGAAGCTCTAATCCATCATGAATTCCCGTTCCCTCAATTCG
GGTCTGGGTCTGGTACTATCACCCGTAAGGACTTGGAGGAAGCCATGTTGCTGCAAATGGAGATGCAGAAGAAACTGCATGAT
CAGTTGGAGACTCAGCGACACTTGCAGCTCAGTTTAGAGGCACATGGCAGGTACATTGCAAGTCTTATTGAGCAGGAGGGTCT
AGGGCAACAGATGCCGGAATTGAGT
>Cylindrocapsa_geminella.PSR1
AGTGGTGATTACGTATACCCAGCAGCCGCACCTGGCCACCTGAGCCAGCTAGGGCCCGGAATGGGCCCCGGGCTTCAGTCCAT
GTCACATAGCATGGGCTTCTTGGCTGAAGGCTCTCATGCGTCTGGCTCTCACGGAATGTACCCGCACTCAACTATTTTTACGA
ACACTCCGTCGAAAGATGGATCGAGGAAAGGCCGACTAAGATGGACTCCTGAACTACACGGTCGCTTTGTTAGTGCTGTAACC
CAGCTTGGTGGTGCGGATAAAGCAACTCCAAAGGGGATTTTGAAAATCATGGGTGTAGAGGGCCTAACTATTTACCATATCAA
GAGCCATCTGCAGAAGTATCGTTTGAACATCAAGATGCCAGAAGCAGAGGCAGGAGGGACAGGCCAGTCAACCGATACTGCGT
TGAAGGTTGAAAGCAACGTGCCAGCAGTAGGTCAGGCCAGGCCGCTAGGCGCTGCAGTTCCTCCGGGCAATGGTTCCTTCAGG
GACATGGGTGCGGAAGTGAACCCGGCTGCTGTTTCTGCACGTGCTGACACCACGGCTGGTCCGTTTGTTGGAGAACAGCGTGG
AGATCAAGTCACACAGCCTGTCCGCGGTCAGGAGCAGCTGCCGCCAGTGGTCCAGCAGAGCAGCGCGGCACCAGCGGACAGTG
TGAACATTCACGAGGTCCTCAAGCAGCACGTAGCACTGCAGCGAAAACTGGCGGAGCAGCTTGAGACGCAGCGCCAGCTTCAG
TCTCACCTGGAGCAACACGGCAGGTTCCTGCGCGAGTTGATAAACACTAGTGCCAGCACGTCG
>Scherffelia_dubia.PSR1
TCCCCGTCTGCATTCAAGCCGCGGCTACGCTGGACAAACGAACTGCACAACCAATTTATAGAAGCAGTGGAGACTCTTGGGGG
GCATGGCAAGGCCACACCATCAGCACTGCTGAAGCACATGAACATGGAGGGCCTGACCCTTGGACATGTCAAGAGCCATCTCC
AGAAGTACCGCACAGAGATTCGCCGTGCCAAGGAAGCTCGGTGCAAGGTCAAAGATGTTCTCAAGGAGATCAAGCGGGACAAA
GCCAGCAAGCCTGGGGCAGGGGGCAAGGCGTTGGATGTTGCAAGAGAGGCGTACGAGGATGGGCCCAATGCGAGGGAGCTCGA
GGAGGCTATGCACACCCAGCTGGAGCTGCAGCGACTGCTGTGCGACCAGCTTGAGGCCCAGAAGAAGCTGCAGAGTAGCTTGG
AGCAGCACACAAAGTACATCTCGGTCCTCATGCGGAACAAGTCGGATGTGCGCACCAAGCCCAGGGACCCACCGGACACAGGC
TCCATTGAACCGGGTTTTCAGGCCGTGGGGGCAGAGAGCGGGCCCAGCACTTCGGAGGCC
>Hafniomonas_reticulata.PSR1
AAGAGCCGTCTCCGCTGGACGCCTGAGCTGCACACCCGCTTTGTTGCCGCTGTATCCAGCCTGGGTGGCCCAGAGAAGGCCAC
GCCCAAGGGTGTGTTGAAGCTCATGGGCGTAGAAGGCCTGACCATCTACCATATCAAGAGCCACCTTCAAAAATACCGCCTCA
ACATCAAGATGCCTGCAGACGGCAAGCAAATGTCCGGCAGTGACATGAGCGGGGCCGTACTAGGAGACCCGAACCGACCAGAG
TTACCCTCAGTCAGCAGCCTTGCCTTAGACACAAGCGAAGCCACACACCTTTCACCGCACTTGCGACCACATGGGAGCCCAGC
TCCCTTGGCGGCCACCAGCGTCCCGTCCCTCAGCAGCCTGCCGGGCTCGATCGATGCGAAGGGCAGCCTAGAGCAGGCGCTGC
TGTTCCAGATGGAGCTGCAGAAGAAGCTGCATGAGCAGCTGGAGGCTCAGCGGCAGCTGCAACACAGCCTGGAGGCGCATGGC
CGCTACATCGCCTCACTGATGGAGGCTGCGCAGGAGGGCGTC
>Tetraselmis_chui.PSR1
CAGAACACCAACTTCCAAATGCCCTCGGGCATGCACTTTCCGAATTTCAACCCAAACGTGCCCGATGGAAATATGCCCAACTT
CGGTTCAAGTTTATTTCCACCGACTACGTTTAGACCGCGGCTGCGGTGGACCAACGACCTGCACAACCAGTTTCTTGAGTCGG
TGGAGCAGCTGGGTGGGCACGGCAAAGCCACGCCATCCGCGATCCTTAAGCACATGGCTGTCGACGGACTGTCGTTAAGCCAC
GTGAAAAGTCACCTCCAAAAATATAGGACCGAGCTGAAGAGGGCAAAGGCGGTGCGGGGGAGGGCACTGAACGATATGAACCA
GATCAAGAAGGGCGCTCGCCGTAGGGCGGGCGAGGGAAGCGGAGGGAGTAGTGCGGAGGAGGGTCTGGATATTTTGGGGTCTA
CGCACGAGGAGCTGCAGAAGCAGCTGGCAGCGAAGGCGAAAGGCCCCAACGCGAAAGAGCTGGAGGACGCCATGCGCACGCAG
CTGGAGCTACAGAAGATGCTCTGCGCGCAACTCGAGGCGCAGAAGAAGTTGCAGAGCAGTTTGGAGCAGCACACAAAGTACAT
ATCGGTGTTGATGCAAAAG
>Volvox_globator.PSR1
GAATTCCTGCCCGTCCTTGGCTTTGACGCATACAGCGCAAAGCCAACTGGACTGGGCTTGGGCGGGCTTCTACCAGACCCGCC
CCGAACATCTACCGATGGAGCATCTACGCTGCTCCAATCCTCAGATTTTATGCTATCCATGCCAGCCGTACCGCACCTGATGC
AGCCCGGCGTAGGAACTTTGCAGCCCCCGCAGTCCGCTTTCCCGGACCTCACGCTCCCAGGCGCCGGTAGCCTGGGGCTCAAC
TCTGGGCTACTCCACCACCCGAGTGGCCACTTCATGGGTCAGCCTCAGCGCGCAGCCACTGCCACGGCCCCCGGTCACGGACC
CGCCAAGAGCCGTCTTCGTTGGACCCCCGAGCTCCACAATCGCTTCGTCATGGCCGTCAACCAACTGGGCGGACCGGAGAAAG
CAACACCAAAAGGCATCCTCAAGTTAATGGGCGTGGATGGCCTCACAATATACCACATTAAGAGCCACTTGCAAAAATACCGT
TTAAATATACGTCTACCAGGCGACGGGGTTCAGGGAGACAGTGCGGCGGATTCGGACATGTCGGACGGGGAACCGGGCGGCGA
CGGATTCGGCGGGCCATCCACGGTTGCGGGGGAAATGCAGTCTGGGCTAGCTGGCGGAGGCGGCGTCAGTGGG
>Volvox_aureus-M1028.PSR1
GGGCGTGCTGCCCTCCCCATGGACAAGGCTGAGCGCGCAGCCAGCAACGCGATTGGCAACGAGGACGACTGGCTCTTGGAGTT
CTGGCCGGAGCCAGCTGCGGCGGACTTCCTTGGCCCAGTTGCGGGCGCGATGCAGCAGCAGCAGCAGCACCCGTTACAATTGG
ACCATTCACAGCTGCCAGAGCAGGTTCCCCACTCTGGAAGTTTTCAGATGAGCCAATTCGGCCTCAGTCCTCCTACCAGCGAT
TACCTCCCGGGGCTCCAGTTCGACGCATACGGCAGCAAACCGCACGGTCTCAGCGGGCTCGGCGGGCTTTTTCACGACCACCA
ACGCTCCTCCACCAACGGTGCATCAACACTGCTCCAACCCTCCGACTTATTGTTTCCCATGTGTGGGGTTACTCACGCACTTA
TGCAACACCCTGCGGGCGTTGCGGGCTTCCAACAGCCCGCTTTTCCAGACCTGCCGCTCGGCGGGGTGGGTCTGCACCCGGGG
TTGCTTCCCGGCCACTATCTGTCGCATCAGCAAAGAGCAGCCTCGTGCCCCGCGAAGAGCCGCCTCCGCTGGACCCCCGAGCT
TCATAATCGCTTCGTGGCCTCAGTGAACCAGCTAGGCGGCCCCGAGAAGGCCACTCCGAAGGGTATCATGAAGCTGATGGGCG
TAGACGGCCTCACCATATACCACATCAAGAGCCACCTGCAGAAGTATCGCTTGAACATACGGCTGCCTGGGGAGACGATGCCC
GGCGACAGTGCAGACACGGATGGCTCCGACGGCGAAGGCGAAGCACCTTCAGCGTCAATGGACAGATTGGACAGGTTGGAAGC
AACGCAGTCGGGGATGTTGGGGGGAGAAGGTGGCGCCGGTGGCGCTGGAGGAGGGGCCACGACTGCCGCAACGGAGCAAACGG
TGTCCATCAGCGCTCAGGGAAAGTCTGGTCGGCGCTCGGGTCCTGCCGGTGGTACTTCTTGCAGCAGCGGCAGTGCCCCCTCA
GCTACGCGGCGCAACCTCGAAGAGGCGCTTCTGTTCCAGATGGAGCTTCAGAAGAAGCTCCACGAACAGTTGGAGACGCAACG
TCAGCTGCAGCTCAGTTTGGAGGCGCACGGCCGCTATATCGCCAGCTTGATGGAACAGGAGGGCCTTACGGGGAAGCTGCCGG
AGCTGACTGAAGCCCCGCTGGGTGGTGGCGGCGCCAGTGCTTCCATTGGAAGCCGGGAGCGCCGGGCTTCCGGCGGCCTAGGA
GCGGGGCTGTCATCAGTACAACAGGCGCCGTTGGGAAGCGCGCCGCCACCACTCACCACGTCTAAAGACCGTGGGGGAAGAGG
GATAGCTGCCGGCCGCGCTATCAGTGGGGGCTGTGGCGCGCTTCAGTCTCCGGCCACGAATCTGAGTGGGGCTTCACCCCATC
TCCAGGCTTCGTCTGGGGGCGTTGCCGGCGTCGGGCTGCAGCCGCTACAGCCGCCACCTGCTGCTGTGGGGGCTGCAGCCGGG
CGGCAAGGCAATCAGCAACAGAAGCCTCAGCATCAGTTCCAGAACCAGCAACAGCAGCAACAACAGCAACAGAAGCAAGTGCA
AGCGGTTGGCAATAGCATTCTCACTGGTGTACGACATAGCCCTCTGCACGGATTGCCATCACTTGGCGGTAGTGGTGGCGGCG
GCCGCGGAAGTGTGACCAGTGTGACCAGTAGTAGCTCGATGCATTTTCAGATGCAGCAGGACCATCAGCGTTTGGAACTTATG
CGGTTGGGCCGACTTGGGTCGCACCCGACCCCGGGGTCACCAAGCGGTAATCCGCTGGTCGATGGTGGCGGCGGCGGAGGAGG
AGCAGGAGTGAATGAGAAGCCTCAGCACATTGTTTCCAACACTGGCCTGGCGGTTGTGGGTGAGAGCAGTATCCCGCTGGAGC
AACCTGTAGTGATACTGCAGGATGGTGGCCATTCTGGGCAAACAGCAGCCCTAGCGCATCAGCAGCCTGAGCCGCCGCAGGTG
CACCCGCAGCCGGCATTGAGGGCAGCGTCGGGACAGCTGGAATCTGGGCTCGGGTTGGGGGATGCATTGGAGGGAATTATAGG
CGAAAGTGGAAATGGCGGTGGTGGTGCCAACGGTGGCAGTGTAGTGCCGCTGCCGGACTTCGACTTTGGAGATTTTCCGGATT
TGGATAGCGGAGGATTGGAGCATCAGGGTTTGTTGGGACCTGGTGACCTG
>Ignatius_tetrasporus.PSR1
GCGAAGCCGCGATTGCGATGGACGCCCGAGCTGCACAAACGCTTTGTTCATGCAGTGCAGCAGCTCGGCGGTCCAGACAAGGC
GACGCCCAAAGCCATTCTAAAGCTCATGGCATCACCTGGCTTGACAATTTTTCACATCAAAAGCCATTTGCAGAAGTTTAGGC
TCAACATCAAACTCCCTGATACGAAGAAAGAAGGTTCAAAAGCGACAGTATCCGGCCAATCAGAGCCGTTTGCGGACCAGGCT
GACAATGCTGTCAGCATGCAGTTTGAGGAGCCCACCAGCGAACCGGAAGTTGCAGTCTCGCCATCAGCAGGCAGCTCGCATGT
CGCTTACAAGGGTCTGCTCGGCAAGAACCTTGGCGAAGCTTTAGTGCGGCAGATGGAGCTGCAAAAGAAGCTTCACAAGCAGC
TGGAGTCGCAGCGACATTTACAATTGAGTTTGGAAGCGCATGGTCGCTACATCGCCGGTCTGATTGCGCGC
>Gonium_pectorale.PSR1
ATGGACAAGGCTGAACGGCAAGCCGCCACCTCAATAGGGCCTGAAGACGACTGGCTGCTGGAGTTCTGGCCAGAGCCTACGGT
TTCCGACCTGCCGCGGTTTGGGCCCGCGATGCAGCCCCTGCACCAGCCCCACCAGCCCCTGGACGCACCCCAGCATCCGGAAC
TCCTCTCACAACAGCAGCAACAACATCAAGCCTTGCAACTAGGAGCGTATGGCCTGCAAGCACAAGCGCCCATGGGATCGGAC
TATGGCTTGCCCGGACTCGGTTTCGACGCGTTTGGCGGCAAGGCCCCGCTGGGCATGGGCGGGATGCTGTCCGAGCACCCGCG
CGCTTCGGAGGGAGCTTCCGCCATGCTGCCCCCATCGGACTTTATGCTGCCAATGGGCGGCGTGGGCAGCATGCCGCACCTCA
TGCAGCCGGGGATGGGTGCGCTGCAGCAGCACTCCTTCCACGACTTCAGCCTTGGGGGCGCAGGGCTGGCGCAGGGCATGCTG
CACGGACACTACATGGCCCACCAGCAGCGAGCAGCGGCGGGCCCCTCGAAGAACCGTCTCCGCTGGACGCCGGAGCTGCACAC
CCGCTTCGTGACCTCGGTCAACCAGCTAGGAGGCCCCGAGAAGGCCACCCCCAAGGGCATCCTCAAGCTGATGGGCGTGGACG
GCCTCACCATCTACCACATCAAGAGCCACCTGCAAAAGTACCGCCTCAACATCCGGCTGCCGGGCGAGGGCGGCATGCAGGGC
GACTCGCCGGTCGACTCGGAGATGTCGGAAGGGGAGGGCGCCGCGCCGTCGGCG
>Planophila_terrestris.PSR1
CAGCCGTCCGCGAGCACCACAGGGAAGTCAGGGCGACTCCGATGGACGCTGGAGCTGCACAAGAAGTTTGCAGACGCGGTGGC
GAAGCTGAACGGCCCCGATAAAGCCACGCCAAAGGGCATACTGAAGCTCATGGACACCAAAGGCCTCACCATTTACCACATCA
AATCGCACCTGCAAAAGTACAGGTCCAACATGCGCACAATGCAGCCGGCGCCCATGCAAGCGGCGCTCTCCGACGCGCACACA
GCAACACATCACGACGCGCGCAGCTTTGGCATGCAGTCCTTTGACGCTGCGGTCTCAGCGTCCGGGGCCATGCATTCGGGCGC
GCTCACATCAAACCCGCTGGGTTTGGGTTTGAATTTCCCGCAGAGCTCGGTCAGCAGCATGGGCGCGATGCTCGGCGGCATGG
GCTCAGCGGCTGCGGGCCCGCTCACGATGACTGGGCTGCCGCCGATCCGCACCGACGCGTCGCTGCTGCGGGCCGCGAGTCAG
GGGCCTAGTCAGGGGCCCGACGCGCTGAGCTGGGCGCATGCGCATGGCACCTCTCCCGCGTTTGCGCGCGGCGGCTCACCGAC
GTGCAGCGTGCACAGCGGGGGGGGGGGGCGGGACGAGGGGCGCGGGGCGTCCGGCGAGGGGGTGGACCAGAGCCACAGCTGGA
CCAAGGCGCTTGTCAAACAGATGGAGCTCCAGAAGCAGCTTCATGAGCAGCTCTCGATGCAGCGGCAGCTGCAGCTCAACCTC
GAGGCGCACGGCCGCTACATGTTGCGGCTCGTGGCCAAGGAGGGC
>Pteromonas_angulosa.PSR1
GGGGGCGCGACAGCCTCTGGAGGCTGCCGGGCGGGTAATGGGCTGGGGAGCGGAATGAGCGAGCCACACGAGCCTCGGGACGG
CAAGCAGCACTTGGGCTCGCTCGACGAGCTTTGGCTGGACGACCACGCCGTAGGCGAGCTTTCCTGGTGGCCGGAGCCCCAGC
TTCCACTACCAGACCCACTGCTGCTGCAAGGTGGCGCAGCGGGCTTGATGCTGGGCCACCAGCCTCAGCAGCAGGCCCAGCAG
CAGCAACCCGCCTACCATGGCCGCATGCCCCCGCAGCAGCAGCAGCAGCAGCCCTCCCAGTTCAACAACGAGTATGGTTACGC
GCCAGTGCCCCATGACCCATATGCACTGGGGGCGCACGCTCAGGCGCCAGCAGGGCCCTCGGGAGACATGTCAGCAGGGGTCA
GTGCAGTGAATTACGCCAGCTTGACCCCTGGGCCTGCGATCCCCCAGCCACACTTGCCACCGCAGCCAATGCCACAGCAGCTG
CAGCAGCACCCTGGCTACATGGACCCGTCGATGATGGGCGGCATGTATGGCGGCCAGTTCTACCCAGCAGCCCCCCAGCAAGG
GCCCGCCAAGTCGCGACTCAGGTGGACCCCTGAGCTGCACAACCGGTTTGTGGCAGCAGTGAACCAGCTGGGCGGTGCTGAAA
AGGCCACCCCCAAGGGCATCCTCAAGTTCATGGGCGTGGACGGGCTCACCATCTTCCACATCAAAAGCCACTTGCAGAAGTAC
CGCCTGAACATCAAGATGCCTGGGGAGGGCACCCCCATGCCCGGCGACAGCGACAGCGAGTCCATCGATGTGGGGGTGCGCCC
CATGCAGCAGCCACAGCAGCAGATGCAGCAGCCACAGCAGCAGATGCAGATGCAGCAGCAGGGTGACACGTCCATGCGCAGCA
AGTCCAGGGGCGACAGCGGGGATGGAGTGCGGGGTGGTGGCGCAGTAGTTGCACCTGCGTCCCAGGCAGCTGGGCCAGGGTTG
TCCTCCTCAACGTCGTCTGCCATCAACCGCAAGAACCTGGAGGAGGCGCTGCTGCTGCAGATGGAGCTGCAGAAGAAGCTACA
CGAGCAGCTCGAGACGCAACGCCAGCTGCAACTGAGTCTGGAGGCACACGGGCGATACATAGCAAGCTTGATGGAGCAGGAGG
GGCTTACGCAGCGCATCCCGCAGCTGCACGGCAATGCACAGATGCCTGCCAGCACAGCCAATGCTGCGGCAGCGGTCGCAGCG
GCGGCTGCGGCGGCAGCAGCAGCATCAAGCAAGCACAACGACCCACGGGGAGCTGCGGCTGGGTCCAGTGCGTCTGGGCAAGG
GGGGCAGTCTGCGGCACCATCTCAGCCTGCATTGGAGCACAGGGCAGCGTCAGGAGCAGGAGCCATGCCCGCGCACTACCAGC
AGCAGCCGCCACAGCAGCAGTACATGCAGCAGCAACAGCAGCAACACCCTGTGTCTGACAGCGGCATGGCCGCCGCCGCGGCA
GCCGCAGCAGCCGCTGCAGCTGCAGCTGGCATGGCAGGCCCTAGCGAGATGTACGCTGGGCACTCCCAGCAGCAGCACCACTC
GTCACAGCAGCAGCAGCAGCCATACCTCCAGCTGCAGCACCGGACATCGCAGCAGCAGGGCGAGCACCAGCCCTCCAGTATAG
CAGCAGCCGGCAGCGCGTGGGGGGAAGCAGCATCCACGGGTCACGGGCCTGGG
>Asteromonas_gracilis.PSR1
GGTCCATCTAAGTCGAGATTACGTTGGACCCCCGAGTTGCACAATCGTTTTGTACAAGCAGTCAATTATCTTGGTGGTCCAGA
TAGGGCAACACCGAAAGGTATCCTCAAGCTTGTCAATGGGGAAGGCCTCACCATCTACCACATCAAAAGCCATCTACAGAAGT
ACCGTTTGAACATCAAGCTACCAATGGATCCCTCGGGATCGGAATATATGAGTGACAGCCAGCAGGACGTGTCAGCCTCAGGA
GAGATGCGCAGCAGTGTAGGGCATGTGGTGACCAGGAGGAGAGCTTCGCCGATGCCCGGTGCTGCCCAAGAAACGCCTCAGCA
ACATGCGACACAAGTGCAGGCAGTTGGCAACACTCAAGAGGGAACATCAAGGTGTTTAGATACTTCAGCCGTTGCGTCGGGGG
ACAAGTCCAAAAGCCTAGAGGATGCGCTTTTGTTTCAGATGGAGCTGCAGAAGAAATTGCATGAACAGCTGGAGTCGCAGCGC
CAGCTGCAGCAAAGTCTGGAGGCACATGGACGTTACATCGCCAGCCTGATGGAGCAGGAGGGGCTA
>Haematococcus_pluvialis-B.PSR1
AAGTCGCGATTGCGCTGGACGCCCGAGCTGCACAACAGATTTGTGCAGTCAGTGACTACCCTTGGGGGCCCAGACCGAGCGAC
GCCCAAAGGCATCCTTAAGCTGATGGGCGTAGATGGTCTTACCATGTACCACATTAAGAGTCACTTGCAGAAGTACCGGCTCA
ACATTAAAATGCCAGCTGAAAGCGGGGGCCAGGACAGCTTGAGCGACAGCCAGGACCAGCAGCCACCCAGCGCCATGGAAGTC
CGAAGCAGCAGTCGTGGGCCAACATCTACACCACAGTTGCGGGCGCCAGGCTCGAGCTACGACTGTAGTGGGCAAGCCCCAGC
TCTGGTCTCGGCAGCGTCTGTCACCGCTGTACCCGCACCGTCTTCGGCCGGTGCCGCCTCTTCAGGCACAAACAGGCGCAACC
TTGAGGATGCACTGCTGTTCCAGATGGAGCTGCAGAAAAAGCTTCATGAGCAGTTGGAGTCTCAACGGCAACTACAGCTCAGC
CTTGAGGCGCATGGTCGCTACATTGCCAGCCTTATGGAGCAAGAAGGCTTGACACAGCGG
>Chlamydomonas_bilatus-B.PSR1
GGGCACCCCGGGCACCATGAGCAGTTCCGAAGACAAAGCGAAGACAAGCCTGGCTCCTCCAAATCGCGGCTTAGGTGGACGCC
CGAGCTGCACAACCGCTTCGTCAACGCTGTGAATCAGCTGGGAGGCGCGGAGAAGGCCACTCCGAAGGGCGTGCTCAAGCTCG
TGAACGTCGAGGGGCTCACGATTTACCACATCAAGAGCCACCTCCAAAAGTATCGTCTCAGCATGAAGCTTCCTGGCGATGCC
GGCGGCGCAGAGAGCCCTGCAGAGTCTGACAGTGGCCTGGAGCAGGGGCAGGGGCAGCAGCCCACGCGGCGCCGCAGCAGCAT
G
>Vitreochlamys_sp.PSR1
GGCATCGGACACAACGTGCTGGCGCTCCAGGGGTTGCCGTCTCAATCACCAGCCCAAATGCTTCCGTTCGTGCCCGACTACAG
CGGCCAATTAGGAGCAGGAGCCTTGCCGCCTGGCCTCCACTTACAACAAGGCGTGCTGAGGAGCAGCAGCGCCGCTCAAGCAC
AGAGAGCCCGTCTGCGATGGACGCCAGAGCTGCACAACCGATTTGTCACTGCCGTCAATGCACTCGGTGACAAAGCAACTCCT
AAAGGGATTCTCAAGCTTATGGGAGCTGAGGACCTGACCATCTATCACATCAAGAGTCACCTGCAAAAGTACCGCCTGAACAT
CCGTCTTCCCAGTGGTCCCCACGCCGATTCTGGGGTGTCCTCAGACAATGACATGGGCGGAATGGGCAATGCGCTGAGCACGG
TGTCATCTGGGCCGCTCGGGGACCTCGCAGATTTCCCACAGCAGCACGAGTTCCAGACGCAGCAGCAAAGGAGCTTGAGCATG
TCGCAGACCCAGCAGCAGCTGTCGCAGCCGCAAGCACCCTTCATACCTGGGACAGCGTCCAGCCCAATGCAGGCAGCGTCTAT
CGCACCCAATCCAGGAGGTTCGTCACCGACAATGCAGCAGATGATGCCTGACCAGCAGCATCGCATGGGCAACCTTGAGCATG
CGCTGCTGGTCCAGTTGGAGCTGCAGAAGAAACTGCACGAACAATTGGAGGCCCAGCGACAGATACAAATGAAGATTGAGGCG
CATGGGCGATATCTGGCGAGTCTAATGGCCTCAGAG
>Botryococcus_terribilis.PSR1
GGCATGGGGGGCCACCAAGCTCCCTTGGGTGGGGCCCCGATCTTCACAACAAGCAGATCAGTGAGTTCCTCTCAGGGCATGAG
AGACCCGGGAAAACAGCGTCTGCGCTGGACGCCGGAGCTGCACGCGCGCTTCGTCGGAGCGGTAAACCAGCTTCACGGGCCGG
AAAGGGCGACGCCAAAAGGCATTCTGAAGCTGATGGATATCGACGGGCTGACAATCTATCACATCAAAAGCCACCTGCAGAAA
TACCGCCTCAACATCAAACTCCCGGGCCAAGGCGGGCCGATGTTTGAAGAGGACGACATGTCGGAGCGGCGCAGCACGAAGAG
ACGGCGCACTAAGGCGCGCTCTACGAAGCGGCGGCGGAAGGCACGGGGTCGAATCTCGGACAGCGAGAGCGAGGAGGACCATT
ACGAGAGCGAGGAAGAGGAGAACGACCCTGAGGAAGTCGAGGCGGAAGAGGAGGAGGACGACGACGGAGGCGAGGTAGCGTCC
CGAGGGACCGGCGCTCCCTCGGGCGTCCCTGTGCCGGTGCCACATCACCCTGCAAGATCGCAGCCCATGCCTACTGCCAGCAG
CACAGAGGACTCGGTGGACGCTGACGGGAAGAAAGCAACCCGCCACGACCGGCAGGCCGCGCTTACTAAGGCCCTCAACGACC
AGATGGAGATGCAGCAGAAGCTGATGGAGCAGCTGGAGTCTCAGCGACGCCTCCAAAGCCAAGTGGAGAGGCACACGGCGTAC
CTGCGGGGGCTTATGGAGGAGGAGGGCCTC
>Eudorina_elegans.PSR1
GGGCGTGTTGCCCTCCCCATGGCCAAGGCTGAGCGCGCATCTGGCACCGTAGTTTGCAACGAGGACGACTGGCTTGTGGAGTT
TTGGCCGGAGCCAGCGGCGGCAGACCTCCTGAGCGCGGTTCCGGGCGCAATGCAGGCGCAGCAGCAACACCCGCACCAACAAC
TGGACCCCTCCCAGCTATCGGAGCTTCTCCCACAGCAGACGGGGCTCCAGATGGGCCAATTTAGCTTGCATCAAACCAATGAA
TACCTCTCAAGCATCCAGTTCGACGCGTTTGGTGGTGGCAAAGCGACTGGACTGGCGGGGCTGGGCGGGTTGCTGCCAGATCA
CCAGCGCTCCTCCACAGACGGCGCTTCGGCGCTGCTGCAGTCCTCAGATTTCATGCTGCCCATGGCCGGCGGCCTGCAGCAGC
CGGCCTTCCCGGACTTAGCGTTGGGGGGCGTGACGCTCAACCCAGGCATGATGCCCGCCCACTTCCTGGGTCACCAGCAACGA
GCAGCGTCAGGACCCGCCAAGAGCCGACTGCGCTGGACGCCCGAGCTGCACAACCGCTTCGTGGCGTCGGTCAACCAACTGGG
CGGGCCGGAGAAGGCGACGCCCAAGGGCATCTTGAAGCTCATGAGCGTGGATGGCCTCACAATCTACCACATTAAGAGCCACC
TCCAAAAGTACCGCCTCAATATCCGCCTGCCCGGCGAA
>Pandorina_morum.PSR1
GGGCTCGGCGGGCTGTTGCCCGATCACCAGCGCTCGTCCACAGATGGCGCGTCAGCGCTGCTGCAGTCTTCCGATTTCATGCT
TCCCCTCGGTGGCGTGCCCCACCTTATGCAGCCCGGCGTCGCTGGCTTGCAGCAGTCCGCGTTTCCGGACCTGGCGCTGGGCG
GCGTGGGCATCAATCAGATGCTCCTGCAAGGTCACTTTCTGGCGCACCCGCAGCGAGCAGCGTCGGGCCCCGCCAAGAGCCGG
CTGCGGTGGACGCCCGAGTTACACAACCGCTTCGTGGCATCTGTCAACCAGCTGGGCGGCCCGGACAAGGCCACGCCCAAGGG
CATCCTGAAGCTCATGGGCGTGGACGGCCTCACGATATACCACATCAAGAGCCACCTGCAGAAGTACAGGCTCAACATCCGGC
TGCCCGGGGAGACCACGCAGGGCGACTCTGCGGACTCGGACGCATCCGACGGCGAGGCAGCGGACCCCTCGGCGTCCATGGAC
CGCACTGTAGAGACGCAGTCGGGACTGGGCGGGGGCTGCGGCGGATCCTTGGCC
>Oedogonium_foveolatum.PSR1
GCCCGTGCAGGCACTGTCAAGGCACGCCTGCGTTGGACGCCAGAGCTTCATACACGCTTTGTGGCATCTGTCCAGAGCCTGGG
CGGGCCAGACAAGGCCACTCCAAAGGGCATTTTAAAACTGATGGGAGTTGAAGGATTAACAATTTATCACATAAAGAGCCATT
TGCAGAAATTTCGGCTTAATATGCGTCTGCCTGAATCGACATCCACCAGCCAAGGAAACGAGGCCGGCACAAGCAGTAAACGC
AGCAAGAAAGATGAGCCTCAAGGAGGCGATTCTCCAGCTGTTGAACAACCGAAAGCCAGTGAGACTTCTACAGCTTCGCAACC
TCCACCCGCTGCGTTGACAACTTCCACGGCAACCAGTGCGCCTGCAGCCGCACTTCATCATGAATTTCACTTTCCCCAACTTG
GCCGTGGTATCGGATCTATCACGCGCAAAGATTTGGAGGAAGCCATGTTGCTGCAGATGGAAATGCAGAAAAAACTCCACGAG
CAGCTGGAGACACAAAGGCACCTACAACTCAGTTTAGAAGCGCATGGAAGGTACATTGCGAGTCTTATCGAGCAGGAGGGCCT
GGCCCAGCAGATGCCTGAATTG
>Chlamydomonas_sp.-M2_762.PSR1
CCGCAGCGCCCTGCTCCCAAAGGCTCCTCCAAGTCACGACTGCGGTGGACTCCCGAGCTTCATAATCGCTTTGTCAACTCAGT
CAACCAGCTGGGAGGACCAGACAAGGCTACTCCCAAGGGCATCCTCAAGTTGATGTCTGTTGATGGCTTGACAATCTATCACA
TCAAGAGCCACTTGCAGAAGTATCGCCTCAACGTGAAGACCCCGGGTGACTCCGCAGCAATGTATGATATGGACTCTGATGGG
GACGGTGAGGGCGAGGTGACAGACACCCGACCGGCGCGCTCCAAGGGTCAGAGCGAGGCGACAACATCTTCGGGTGGGACAGC
CAGGGGCAAGCACAGCAACCGGCAGCATCAGGCTGCCTCGGCGCCCGTGGGCCTTCCTGCGGCAGCGCCAGCGCCACCGGTGC
CTGGTATGACCACCGCCGCGTCGCTGCCTGTGGTGTCCAGCAACAACCGCAAGAACCTAGAAGATGCGCTGCTCGTGCAGATG
GACCTGCAGAAGAAGCTTCATGAGCAGCTGGAGAACCAGCGCCAGCTGCAGGCCCAGCTGCAGGCACACGGGCACTACATCGC
CAGCCTCATGCAGCAGGAGGGCATGGCCACGCCCGCTGAGACGCAGCCCCCAGCGCCAGACACCAAGCCACCGGGCCTGCCCA
GCACCTCCGCACCGGCTGGGCTCCCGGGCCCACTTCCACCT
>Chlamydomonas_sp.-M2762.PSR2/homologue
GGTGTCTCCAAGTCAAGGCTGCGTTGGACTCCAGAGCTACACAACCGCTTTGCAGCTGCTGTGAGGCTGCTGGGAGGACCAGA
CAAGGCGACGCCAAAGGGCATCCTCAGCCAGATGAGCGCGCCTGGGCTGACTATTTACCACATCAAGAGCCACCTGCAGAAAT
ACCGCCTCAGCAGCAAAAGCCCTGGCAACTTCAGCCTGAACGATGACTCTGACGACGGGCTTGCAGGGGAGGGAGACGAGGAC
ACGAGCTGCATGGCCAGTGGCCACCGCCAAGACTTTGCCGCTGCAGCGCTGCCCGGCGATGCTGACAGGCGAGCGGCACATCC
TGGCTCACCCAGACGAGCGATCGTCACCAACATGGAGGTGTCAGGCAGCCCGGCCCCGTCAGTGCGGCCGCAAGCAGCGGTCA
TGACAGCTCCGCGCCTGCCTGATGCCGCCGCGAGCAATCGCAGGAACCTGGAGATGGCTCTGCTGAGGCAGATGGAGCTGCAG
AAGAAGCTGCATGAGCAGCTCGAGGCCCAGCGCCACCTGCAGCTGAGCCTGGAGGCTCACGGCCACTACATCGCCACGCTGAT
GCAGAAGGAGGGCTACGCCGGGGGCCCGACGCCCCCGGAGCCCGCTGCGGGAGCCTGCCCGGCCCCAGCCACAGCGGCGGCTG
GCACGGCCGTGATCAGCTCAGCCGTTCCCCAGGGCCTCGCCAGGGCCTGCTCATCA
>Chlamydomonas_noctigama.PSR1
TATCAAATGCCAGGTATTGTCGGCGCTGCACCCACGAAGAAAGGCAGACTGCGTTGGACACCGGAATTACATGCCTGCTTCGT
CAATTCGGTCCACCAGCTGGGAGGATTTGAGAAAGCGACACCGAAAGAAATCCTGAGACTAATGAAGACCGAGGGCATAACGT
TGTACCATATTAAGAGCCATCTTCAGAAGTACAGGCACTGCATGAAGCTCGGAAGACTTGGGGGCACAGACAGCTCAGATGCG
TCGGAAAACCTCCCGGGCGATCAACAGTCTCCCCAACCGATACTGGATTGCCATATGCCTGGACGGACAGACGGGAGTTTGGA
GGTCGCGCCATCGCGGCCAGCGGATGGAGGACGTACGACATGTCATCGTCACAATGACAGCACCCGACAGTTTTCAGATGCAA
ATGTGCAGGCCTCTGCGTCCTCCTGCAGCATTCGCCGCACCGCACTAGAGGAAGCAATTGCTCTGCAGAAGGAACTGCAGAAA
AAATTCCGGGAGCAGATGCAGACGCAGATAGAGCTGCAAGCTCGTCTGGAGGCCCATGGCCGCTACATAGCGACGCTGGTTGA
GCGT
>Carteria_crucifera.PSR1
GGAACACCGAAGTCCCGTTTGCGATGGACTCCCGAGCTGCATAACCGGTTTGTAAACGCAGTAAATCAGCTCGGTGGCCCGGA
GAAAGCAACACCGAAAGGCATTATGAAGCTCATGAGTGTGGACGGCCTTACAATATACCATATAAAAAGCCACTTACAAAAGT
ACAGACTAAACATTCGACTTCCGGCTGAGTCGCAGCTTACAGATAGCAGCACCGAGAACAAACACGAGCTGCAAGGCCAGTCG
CCAGTTCAAGAACCGCAACAGCAGGAGAGAGATTGCGGTGGAACGTCAGCAATACCGTGCGAATTAACAGTTCCTACGACAAC
GTCAGGAAGCGGTGCAGTAACGACCGTTCCCAACGCCCTGTACACTAACGTCCAAGCCTCCATCGTCGCTTCGCAAGCCTCCA
TCGTCGCTTCGGCCCACCCACCCCCGTCAACCGCCGAGCCACCGGTTCAGGCCGGGCCCAGCTCATCGGAGCGCAGGTCCTCC
CCTGAACCCTCTTCCTCGACTAGGAAAAATCTCGAAGAGGCGCTGCTTTTCCAAATGGAGCTCCAGAAAAAGCTGCATGAGCA
GTTGGAGTCTCAACGGCAGTTGCAGTTGAGCTTAGAAGCTCACGGTCGTTATATTGCTAGCCTGATGGAGCAGGAGGGTCTTA
CGCATAAACTACCGGAACTCACAGGGCAAACGTTAGGAGCACCTTCATCA
>Volvox_aureus-M2242.PSR1
GGGCGTGCTGCCCTCCCCATGGACAAGGCTGAACGCGCAGCCAGCAACGCGATTGGCAACGAGGACGACTGGCTCTTGGAGTT
CTGGCCGGAGCCAGCTGCGGCGGACTTCCTTGGCCCAGTTGCGGGCGCGATGCAGCAGCAGCACCCGTTACAATTGGACCATT
CACAGCTGCCAGAGCAGGTTCCCCACTCTGGAAGTTTTCAGATGGGCCAGTTTGGTCTCAGTCCTCCTACCAGCGATTACCTC
CCGGGGCTCCAGTTCGATGCATACGGCAGCAAACCGCACGGTCTCAGCGGGCTCGGCGGGCTTTTTCACGACCACCAACGCTC
CTCCACCAACGGTGCATCAACACTGCTCCAACCCTCAGACTTATTGTTTCCCATGTGTGGGGTTACTCACGCACTTCTGCAAC
ACCCTGGGGGCGTTGCGGGCTTCCAACAGCCCGCATTTCCAGACCTGCCGCTCGGCGGGGTGGGTCTGCACCCGGGGTTGCTT
CCCGGTCACTATCTGTCGCACCAGCAAAGAGCAGCCTCTTGCCCCGCGAAGAGCCGCCTCCGCTGGACCCCCGAGCTTCATAA
TCGCTTCGTGGCCTCAGTGAACCAGCTAGGCGGCCCCGAGAAGGCCACTCCGAAGGGCATCATGAAGCTGATGGGAGTAGACG
GCCTCACCATATACCACATCAAGAGCCACCTGCAGAAGTATCGCTTGAACATACGGCTGCCTGGGGAGACGATGCCCGGCGAC
AGTGCAGACACGGATGCCTCCGACGGCGAAGGCGAAGCACCTTCAGCGTCAATGGACAGATTGGACAGGTTGGAAGCAACGCA
GTCGGGGATGTTGGGGGGAGAAGGTGGCGGTGGTGGCACTGGAGGAGGGGCCACTACCGCCGCGACGGAGCAAACGGTGTCCA
TCAGCGCTCAGGGAAAGTCTGGTCGGCGCTCGGGTCCCGCCGGTGGTACTTCTTGCAGCAGCGGCAGTGCCCCCTCCGCTACG
CGGCGCAACCTCGAGGAGGCGCTTCTGTTCCAGATGGAGCTTCAGAAGAAGCTCCACGAGCAGTTGGAGACGCAACGTCAGCT
GCAGCTCAGTTTGGAGGCTCACGGCCGCTACATCGCCAGCTTGATGGAACAGGAGGGCCTTACGGGGAAACTGCCGGAGCTGA
CTGAAGCCCCGCTGGGTGGTGGCGGCGCCAGTGCTTCCATTGGCAGCCGTGAGCGCCGGGCTTCAGGCGGCCTAGGCGCGGGG
CTGTCCTCAGTGCCACAGCCGCCCTTGGGAAGCGGGCCGCCACTACTCACCACGTGTAAGGACCGGGGGGGAAGAGGGATAGC
TGCCGGCCGCGCTGCCAGTGGGAGCTGCGGCGCGCTGCAGTCGCCAGCCACGAATCTGAGTGGGGCTTCACCCCATCTCCAGG
CTTCGTCTGGGGGCGTTGCCGGCGTCGGGCTACAGCCGCTACAGCCGCCACCTGCTGCTGTGGGCGCTGCAGCTGCGGCAAGT
CAATCAGCAACAGAAGCCTCAGCATCAGTTCCAGAACAGCAACAGCAGCAACAGCAGCAACAGCAGCAACAGCAGCAACAACA
GCAACAGAAGCAACAGCAGCAACAGAATCAGGTGCAAGCGGTTGGCAATTGCATTCTCACTGGTGTACGACATAGCCCTCTGC
ACGGATTGCCATCTCTTGGCGGTAGTGGTGGCGGCGGCCGTGGAAGTGTGACCAGTGTGACCAGTAGTAGCTCGATGCATTTT
CAGATGCAGCAGGATCATCAGCGTTTGGAACTGATGCGGTTGGGCCGACTTGGGTCGCACCCCACCCCTGGGTCACCAAGCGG
TAATCCGCAGGTCGACGGTGGCGGAGGAGGAGCAGGAGTGAGTGAGAAGCCTCAGCAAGTTGTTTTCAACCCTGGCCTGGTGG
TTGTGCGTGAGAGCAGTATCCCGCTGGAGCAACCTGTAGTGATACTGCAGGATGGTGGCCATCATGGCCAAACAGCAGCCCTA
GCGCACCTGCAGCCTGAGCCGCGGCAGGTGCACCCGCAGCCGGCATTGAGGTCAGCATCGGGACAGCTGGGATCGGGGCTCGG
GTTGGGGGATGCATTGGAGGGAATTATGGGCGAGAGTGGAAATGGCGGTGGTGGTGCCAACGGTGGCATTGTAGGGCCGCTGC
CGGACTTTGACTTTGGAGATTTCCCGGATTTGGATAGCGGAGGATTGGAACATCAGGGTTTGTTGGGACCTGGTGACCTG
>Phacotus_lenticularis.PSR1
CCCAAGTCTCGCCTCAGGTGGACGCCGGAGCTACACAATCGTTTCGTGTCAGCAGTGAACCAGCTGGGGGGTGCTGACAAGGC
CACACCCAAGGGCATTCTGAAGCTGATGGGAGTGGATGGGCTCACTATCTTCCACATCAAGAGCCACCTGCAGAAGTACCGCC
TGAACATCAAGATGCCCGGGGACAGCAGTATGCTTGCAGGCGACTCTGACAGCGAGTCCATCGACCCGCAGCGCAGCCTCCGC
ATGCCCGAGCCCATGCGCAGCAAGTCCAAGGGCGACAGCGGGGACGCGCAGCGGGGCCCAGCAGTGCCGTCGGGTGCCCCAGC
GCCAGCAGGGCCCAGCATGCCGGCGCCCTCGCCTTCTGGCGCCGGGCCCAGCATGCCCGCGCCCTCGTCGTCCACGTCGTCTG
CCATCAACCGCAAGAACCTGGAGGAGGCGCTGCTGTTCCAGATGGAGCTGCAGAAGAAGCTTCATGAGCAGCTTGAGACACAA
CGGCAGCTGCAGCTCAGTCTGGAGGCGCATGGCCGCTACATAGCCAGCCTGATGGAGCAG
>Stephanosphaera_pluvialis.PSR1
GGACAGGCGTTGATGCAGCCCCAATTCTCATCGCAAGCTCCCAAACCCGACCCAGCTGCGCCCAAGCAGCGGCTCCGATGGAC
ACCCGAGCTGCACAATCTATTCGTTCAAGCGGTGGACCAGCTGGGAGGCCCAGAGCGTGCCACACCCAAGGGCATCCTCAATT
TGATGAGTGTCGAAAAGCTCACCATTTACCACATCAAAAGTCACCTTCAGAAGTACCGTCTCAACATCAAGTGCCCTAATGGC
GATTCTGGTGCAGCTGGAGACAGTGACAGCTACGACCAGGCACCATCAGGTGGCGTGGTTGAAGGGCGCAGCCTCAGCAGGGG
CTCAGTGCCCACACTCACACACCCACATGCTTCCCTGACCAGTCTTTTGACCGGCAGCATGCCTTCAGCAGCAGCCACTTCAC
CCCAGGCAGTCACACCAGCAGCATCAGCAATCCACGTGCATTCACCCCCACCTGTGGGCTGCCCTACCCAGCCGTCTCTGCAG
CTCACTGTAGGCACAAGCGCACCTGGCAGCCTCAGCCAGGCTCCTGTGTCCCAGCTCAGCAACATGTCTGGTGGGCTGTCTAC
CACTGTCACCGCTGCCAACAGGAAGAACCTAGAGGACGCCCTGATGGTCCAGATGGAGCTGCAAAAGCGCTTACATGAGCAGC
TGGAGCAACAACGGCAGCTACAGCTCAGTCTTGAGGCGCATGGCAGGTACATAGCCAGCCTGATGGAACGAGAGGGCATGACA
>Chlamydomonas_eustigma.PSR1
ATGGCTGACCCCATCGGCCAGCCCAGCCTCCCAACAGACGACCCTCTGTTATTAACATTGAAAACTGGAGGGGTCGACGAACC
TGACCTCGATTTTAATTCTTGGTTAGAATTCTGGCCTGAATCCGAACTCCCGAGCATGCACAGCTTTTTACCTCAAGCAAATA
ATGTGCCAATAGATGAATCTTACCGACAAGGATTTGCCTTGCAGTCAGCAATACCAGACATCCCACGGATGCAGGGCGGTCTC
CTAGATAACTATGACAGAGTACCGACCCTCTTGTCAGCTGGATCAGCGAGAGACATGCATATGCTAGCAGATTTTCATGGTAA
TATGAAGTCTCTTCCACATCATGCTGGTTTCTCTTCGTTCAGTGGAGACATGGGGCCTTCTGCTGGGTCGTATTACCATGAGA
AGGAGAGTAGACACGGCTCAAATTCAAGGTCTCGTCTTCGATGGACGCCTGAACTCCACAACCGCTTTGTGAACTCAGTAAAT
CAGTTGGCAGGGCCTGAGAAAGCCACCCCAAAAGGCATCTTGAAGCTCATGAATGTGGAGGGATTGACAATCTATCACATAAA
AAGCCATTTGCAGAAGTATCGCCTCAACATCAAAATGCCAGGAGACATGAACTTGGAGAGCTGTGGTGATGACTCAGACATGG
ATGAAAGACCCACATCCACTACGCCTGTGGACAGGAACCGCCGAGCTCCTGACCTCGAGCGCCAAGTCTCCCTCAATATGGAC
AGACTGGGGAGAGCTGGCAAGGTTGCTAGTGAGAGGGAAACTCTAGATCAAGGCAGGAGAGGCATGGACACATCATCAGGCTT
GGCAGGCCCAGCTACTGGCCATACAGAGTCAACGCCTCCTCGACCCTCCACAAGTGCTGGAACCACGGCTCCACTCTCGTCAT
CATCATCAGCACTGAACCGCAAAAATCTAGAGGATGCTTTGCTGTTCCAAATGGAGCTACAGAAGAAGCTGCATGAACAACTT
GAGACTCAGCGCCAGCTGCAGCTCAGTCTAGAGGCCCATGGGCGGTACATTGCTAGTTTGATGGAGCAGGAAGTTCTGGCATC
CAAGCAGGATGGTCAACAACCTTCTACTGAGCCCTCACTTGGTGGAGGAGGGGGGACAACCCGGGGTGGGATCACAGCTGCTG
GCTTGTGCAGACCTCCTTCTGGGGCCTCTGATGAAGTCATGATAGCTGATAAGGCAGGGATGGTGTCAACCTCAGGTGCAGCA
TCACCGCAGTACCTCGATCATGGTGGTGCAAAGCAAGGAGGAGCCCACCTGCAGTACTTCAGCTCCGCAGGCAGTGAGGCCTC
TGCCTCTGCTGCAGGCACAGGCCTGCATGGCCTTTCAGTCTCTGCAAGCAATGGTGCTCATGGAGCCATGCTGTCAGGTAATC
AGTTTATGGGCATGGGAGGGGATAGCTCCATGGCGCTCATGTCTCCAGGTAAGCACATGGAGACTCACATGGGTGGAACCGTC
ATGCATGGGAAGCAGCTGCATGGTGTCATGCGAGGAGGCATGGTTGGTACAGCTGCAGCACCCAGTCCCCCCCTCCTCCCGCT
CATGGACGCCCATCCCTCCCATGACGGCTCCCCTGGTGCCTCCTTGTTGCCTACCTCCCTCATGATGGTTCAGCAGTCGTCCC
CAGATCTGGAGCTATTGGTACATGAAGCAGGCAGCCTGGCTTCAGCGGGAGACTGCAACCATGCATCTAAGCGCATTAAGTTA
GAAAATGAGTTATGA
>Chlamydomonas_incerta.PSR1
ATGGACAAAGCTGAACGCGCTCCTGGTGGCCCTAACGCCGCAAGCGAGGACGACTGGCTGCTGGAGTTCTGGCCGGAGCCTGC
CGCGGACTTCCCTGCGCCGGTCGCAGCGATGCAGCCGCAGCATCAGGACGCTACACAGCTGCAGGAGGCCGTTCCGCAGCAGC
AAGGGCTCGCGCTGGGTGCATATGGCCTCGCCCAGCAGCCCTCTGACTTCATGCAGTCAAGCATGCCCGGCTTTGACGCGTTC
GGCAGCGGGAAGGCCGCAACCCTCGCCGGGCTGCCCGGCCTGCTGCCCGACGCCCAGCGCGCCTCCACCGACGGCGCCTCTGC
GCTTATGAACGCGGCGCAGCAGTCCTCGGAGTACATGCTGGCCGCCGGCATGGGCGGCGTGCAGCACTTGTTAGCACCGAGCG
TTGGCACGGCGCTGCCCGGCAGCGGGCACACCGGCTTCGCGGACCTGTCGATGGGGGGCCTGGCGGGCGGCCTTGCGGGCCTG
GGGGGGCCGGGGATGATGCACCACGGGCAGTTCTTCATGCAGCCGCAGCGAGCAGCCACAGGCCCCGCCAAGAGCCGGCTGCG
CTGGACGCCGGAGCTGCATAATCGCTTCGTCAACTCGGTCAACAGCCTGGGGGGCCGGACAAGGCCACGCCCAAGGGCATCC
TCAAGCTCATGGGCGTGGACGGCCTCACCATCTACCACATCAAGTCGCACCTGCAGAAGTACCGCCTCAACATCCGGCTGCCG
GGCGAGAGCGGCGTGGCGGGCGACTCGGCGGACGGCTCGGACGGCGAGCGCTCAGACGGCGAGGGCGGCGGCGGCGGCGGGCG
GCGCGCCACGTCGCTGGAGCGGGCGGACACGATGTCGGGTATGGCCGGCGCCGTGGCGGGCGGGAGGCCGGGCGGGGCGCTGC
TGTCGCCGGGGCTCGCCGGCGCCACGACGAGCACCGGTGCGGTGGGCGGCGGCGGCGGCTTGATGACCGAGCCCAGCATCTCG
CGGGGCGCGGTGCTGAACGCGGCCGGCGCAGCTCCCGCGGGGGTGGTGGCGGCGGCGGTCGGCGGTTCGGCCGGCGTGAAGCG
GCCGGCGGGGACGTCGCTGAGCAGCGGCAGCACGGCGTCGGCGACGCGGCGCAACCTGGAGGAGGCGCTGCTGTTCCAGATGG
AGCTGCAGAAGAAGCTGCACGACCAGCTCGAGACGCAGCGGCAGCTGCAGCTCAGTCTGGAGGCGCACGGGCGCTATATCGCC
AGCCTGATGGAGCAGGAGGGCCTCACCTCGCGCCTGCCAGAGCTCAGCGGCGGCGGACCAGCCGCGCCGCCCGCTGGCGCGGG
CGGCGCCGCCGGCGGCATGATTGCGCCGCCGCCGGCGCAGCAGCAGCTGCAGCACCAGCCGCAGCAGCTGCTGCAGCCGCAGG
GCAGCCTGCCGGCCGGCGGTGGCTCCGGCGTAGATGCTGGCAGCGGCGGCGGCGGCATGAACCTGCAGCCACAGCACCAGCAT
GTCCACCTCCATCACCACCAGCAGCAGCTGCAGCCGCTCGCGCGCCATTGCGACACGTGTGGCGCCGGCGGCGCTGGCGGGGC
GCCCAGCGGCGGCAGCAGCATGCAGCAGCTGCAGGCGGCCGAGCAGCAGCGCACTGAGTTGGTGGCGACGGGGCGGCTCGGGT
CCATGCCCGCGCCCGCGTCTTCGTCACCCCTCGCGGGGCAGCAGCACCAGCCGCTGGCCGGCGGGGCGGCGCACGTGCACATG
CACGCGCACACGCCCGGGGCGCAGCCGCAGCCGCACGTGCAGCGGCAGGACTCATATGCTGGCGCGGCCGCTGCGGCGGCTGC
GGCGGCTGCTGCAGCATTGCCGCAATCGCATTCACACACGCTCCCAGCCGACCTGTCCAGCAACGCCGTCGCTGACCCAAGCG
CAGGGCCGATCAAGCCTGAGCAGGGCCTGTCGCCGCAACAGCAACAGCAACAACAAGAGCAGCAGGAAGCGGAGCAGCTGGCG
CAGGGGTTGCTCCATGACAGCAGCGCCGGCGCAGGGGCTGTGAGCGGTAGCGACGGCGGCGGGCTTGGCGACTTCGACTTTGG
CGATTTCGGGGACCTCGACGGCGGCGCCCAGGGCGGCCTGCTTGGCGCCGGGGACCTGATCGGCATCGCGGAGCTAGAGGCGG
CGGCCGCGCACGAGCAGCAGCTGCAGCAGCAACAGCAGCAGCAACAGCAGCAGCAACAGCAGCAGCAACAGCTGCAGGGACAA
GAACAGGAGCAGCTGGATGCGGACCGCGCAAAACGGCAGCGGCTGGAGCAGTAG
>Chlamydomonas_schloesseri.PSR1
ATGGACAAAGCTGAACGCGCTGCTGGCTGCACCAACGCTGCGAGCGAGGACGACTGGCTGCTGGAGTTTTGGCCGGAGCCTGC
CACGGACTTTCCAGCGCCGGGGGCAGCGATGCAGCCGGCGCATCAGGACGCGACACAGCTGCCAGAGACCATTCCGCAGCAGC
AAGGGCTGGCGCTGGGCGCATATGGCCTCACCCAGCAGCCGGCGGACTTTATGCAGTCGGGCATACCCGGCTTTGACGCATTC
AGCACTGGGAAGGCGCCCGGGCTGGCCGGACTGCCGAGCCTTCTTCCGGACCCGCAGCGCGCCTCCACCGACGGGGCATCAGC
GCTGATGACTGCGGCGCAGCAGCCCTCAGAGTACATGCTGCCGCCAGCCATGGGCAGCGTGCCGCACCTTCTGGCGCCAAGCG
TTGGCACGGTGCTGCCCGGCACGGGGCACACAGGGTTCCCCGACCTTTCCATGGGCGGCATGCCGGGCGGCCTTGCCGGCCTC
GGGGGGCCCGGCATGATGCATGGGCAGTTCTTCATGCAGCCGCAGCGAGCAGCCACGGGCCCCGCCAAGAGCCGGCTACGCTG
GACGCCTGAGCTGCACAACCGCTTCGTGACGGCGGTCAACCAGCTGGGCGGGCCCGACAAGGCCACGCCCAAGGGCATCCTCA
AGCTCATGGGCGTGGACGGCCTCACCATCTACCACATCAAGTCGCACCTGCAGAAGTACCGCCTCAACATACGGCTGCCGGGC
GAGAGCGGGTTGGCGGGCGACTCGGCGGACGGCTCCGACGGCGAGCGCTCCGACGGCGAGGGCGGAGGAGGCGGGGGCGCGAT
GGGGCGGCGCGCGAGCTCGCTGGAGCGGGCGGACACTGCCTCGGGCATGGCGGGGCCCGGCGCGGTGGCGCCGGGGGGGGCGG
GCAGCACGCCGGGAGGGCAGCTGCTCTCACCGGGCGTGGGGGCCACCGCTGGCGCCATGGCGGCGGCAGCCGAGCCGTCCATG
TCCAGGGGCGCGGTCCTGGGCGCGTCGGGCGCTGCCGCAGCCGCGGCGCCGGCGGCGGCCGGGGCGGCGGCCGGCGGCGTGAA
GCGACCGGCTGGGGGGCCGTCGCTGAGCAGCGGCAGCACGGCCTCGGCGGCGCGGCGCAACCTGGAGGAGGCGCTGCTGTTCC
AAATGGAGCTGCAGAAGAAGCTGCACGAGCAGCTGGAGACGCAGCGGCAGCTGCAGCTGAGCCTGGAGGCGCACGGCCGCTAT
ATCGCCAGCCTGATGGAGCAGGAGGGCCTCACCTCGCGACTGCCCGAGCTCAGCGGCGGCGCGCCCGCGGCGCCGCCCGCGGC
CCCCGGCGGCATGCTTGCGCCTCCGCCCCAGCCGCAGCAACAGCAACAGCAACAGCAGCTGCTGCAGCCGCAAGGCAGCCTGC
CGGCCGGTGGGACCACCGCCGGGTTGGACGCGGCTGCCGCCACTGGTTGTGGGGGAGGCGAAGGCCCGCAGGTGCAGCAGCAG
CAGCAGCAGCAGCACCATGTCCGCCACTGCGAGACGTGCGGCGCTGGTGGCGCCCCCAGCGGCGGTAGCAGCATGCAGCAGCT
GCAGGCGGCAGAGCAGCAGCGCAATGAGCTGGCGGCTGCGGGGCGGCTGGGCTCCATGCCGGCGGCCGCGTCCTCGTCGCCGC
ACGCGGGGCACGCGCCGCTGCAGCCGCCGGGCGCACATCTGCATGTGCCCACGCCTGGGATGCATCAGCAGCAGCAACAGCAC
ATACAGCCGCAAGACTCGTACGCTGGCGCAGCTGCGGCGGCAGCAGCACAAGCGTCACCGGCGGCGCTCCCACAGTCGCATTC
GCACACGCTGCCAGGGGACATGAGCAGCAGCGGGGTCATGGAGCAGCCAGGTCCAGGGCTGGTTAAGGCCGAGCCAGGCCTGT
CGCCGCAGCAGCAGCAACAGCAACCGCAAATGCAGCAAGACGCAGATCAGGGGTTACTCGGCGACGGCGGCGCCGGTGCAGCG
GGTGTGAGCGGCAGTGACGGCGGCGGCCTAGGCGACTTCGACTTCGGGGATTTTGGGGACCTGGACGGCAGCGCCCAGGGTGG
CCTGTTGGGTCCTGGCGACCTCATTGGCATTGCGGAGCTGGAGGCAGCGGCCGCACACGACCAGCAGCAACAGCTGCAGCAGC
TACACCAGCAGGAGGAGCAGGAACAACTGGATGCGGATCGCGTGAAGCGGCAGCGGCTGGAGCAGTAG
>Chromochloris_zofingiensis.PSR1
ATGGACCAGCGCAGGGAGCCCGCTGCAGCTAGTCGTGACGCTGATACAGCTGATATCAATTGGTTAGAGTTCTGGCCAGAAAG
CGAGTTTAAAATCGACGGTGCTCCGGCAGGTGGCAGCATGGACCCGAGCTTGGGCACCTTAGGAGGACTTGGGGATTATCTGG
GCTCTAATCTGCAGCACCCCCAGCTGGCAACAGCTGCGCCGCTGCAACTCACCCTGCCAGGAGAGTTTGGCAGCGCACAGGGA
CTTCCCTTGCTAAGCTCTCTGGAAGCGTATCAGGGAAGTGGAGATCTGAATGTGTTACAAAGCACGCAACCAGGGCAGTTGCC
ACAGCTGCTGTCATCAGCACCCTTAGCTGGACATTCACTGTCGTCATCATATGGGTCTGACCCCTCAGGTTTCACTGCCAACC
TCACATCTCCAGCACTATACCCCACTGGGTCGTATCTTGCTCAAAATTCAAAGCCAGGTTTGCCGCCGAAAACGCGCTTGCGA
TGGACTCCTGAGTTGCACAGCAGGTTTGTATCAGCCGTGCATCAGCTTGGTGGGCCAGATAAAGCAACTCCCAAGGGCATCCT
TAAGCTCATAGGGGTAGATGGGTTGACCATATTTCATATCAAAAGTCACCTGCAGAAGTACAGGCTCAACATCCGACTACCAG
AATCTGGACGATCAGATTCCCAGGGTGGTTCAGAGCCCCTGGAAGGTGGCTCAGGTGCAGACAGCAGGATGAGAGCACCCAGC
TCCACACAAACACAAGCGAAACTAGGGTTGAGCAATCAACTAGAATCAGGTCAAGCAGGAACCGAAGCTACACCCTCAGCTAC
AGCTGCATCATCGCAGGACTATGCTGCCGCCCACACAGTGTCGCGACCACGCAGCTCAAATGCTGGCCCCAGCAGCGGTGCAG
GCAAAGCAGCTGTTGGGGATCAGCTGCCAGCAGCTGGCGGATCAGGTTCATACTCGACGGTGGGACAAGGTGGGGCAAAGCca
gcagcacagaaacagcagcaattgaagcagcagTTGACAGCTGGGTTGCCAATATCTGCTGTCACGCGAAAAGACCTAGAGGA
TGCGCTCTTACTACAAATGGAGCTGCAGAAGAAGCTGCATGAGCAATTGGAGTCTCAACGCCAGCTACAACTGAGTCTTGAAG
CCCATGGCAGGTACATAGCCAGCCTGATGGAACAGGAAGGCTTGGGGCACAGAGTACCTGACATAGCTCAGCTTACTGGTGTA
CAGCCGGAGCATTTGCAGCACAAAGAGCAGCAAGCAGGCGGTGTGCCTCTGTCATTCACCCAGTCACTGACAGAAGACCTCAA
TGTTGATGACTCAGCTTTACATATGTTTCCAGGGGGTCATTCAGCAGGTCCTAGTCATAGGCATCAGCATGCGACAGAACAGG
GTCTGCCAGATTCTCCACACCTTCTTTTGAACTTCCCCGAACTCAATGAGCTAGCAGATGTCCCACAAGCAGGTGCAGGCAGC
AATATGTTGTTGCCACACATGCCCATGGGACAGCAGAATCACATGCAGCCAACCCACAAACGGCAGCGCTTAGATGAGGGTGG
CAGCGCGCAGAACCACGGCAGTCATTCTGGTTCACGACAACAACACTGA
>Coccomyxa_subellipsoidea.PSR1
ATGCAGCAGAACCAGCACTACAACTGGGTATCACAGCATGTCAGCGTTGAAGATCATCAGGAGCCGCATACGCAGCATCAATT
GCCGCAAACAGTAGGACACGAGCACCTCACAGCAGCTCACGCTGCCTTCTCAGACTTTGGGCAGCACAGGGGCGATCAGGCGA
TTGCAGGGATCACAGGGGATCAGGCAGAACTGTTGGAGGCCATATCTGGTACAGAGACAGAACTGGGGCTGCCATACACTGCC
CTTGATCATCTGCAGCCTCACCACCCCCAGAACGACTTGCATCAGCATGGGGTTGGCTATGGGATGGAGAGAAATCCATCAGA
GCCCACACAGTCGCAGGGGAAGGATGCGAAGCCGCGCTTGCGCTGGACGCCAGAGTTGCATGCGCGCTTCGTGTCAGCAGTGG
CTTCCTTGGAGGGGCCGGACAAGGCAACGCCGAAGAGCATCCTGAAGCTGATGGCAGTGGAGGGCCTGACCATCTACCACATC
AAGAGCCACTTGCAGAAGTACCGGCTCAACGTGCGGCTGCCGGGCGAGTCCGGTGACATGATCAGCGGCCCCGACGAGTCTGA
GGAGCCCTCGCGACGCAAGCGGCGCAGCCGCTCGCATGGCCAGGCCTCCAGCCGCAGGCGCAGCAGCCGGCAGCGCAAACGGC
GCAGGTCGTCGGACGAGGACAGCGATGAGGACGACATGGAGGATGAGGATATGGATGACGACGAGAACTTCGAGGAGGGCATC
AGCCGCGCGCGGCCGGGCACCAGCGTGAGCGGCATCAACGGCAGCAGTCCCCACGGTGGCAGCCCGCGCGGTGGCAGCCCCCG
GGGGGTCAGCCCCCGGGGCCGCAGCCCGCGAGGAGGGAGCCCGCATGGAGGAGGGAATAGCATGGCCTGCCTGCAGGTGGAGC
CGCTGGAGGTGCCTGACCTGGACCCCGAGAAGCAGCACAGCCTGGAGGAGGCCCTGCTCAAGCAGATGGACATGCAGAAGCGG
CTGCACGAACAGCTGGAGGAGCAGCGGCGGCTGCAGTTGTCGCTGGAGGCGCACGGGCGGTACATCACCAGCTTGATACAGAA
GAAGGGGCTGGAGGGGCTTCCTCCGCAGACCAAGGAGGCTTTAGATGCCGCCCTGGTGCCCCCACAAGGCTCAGGGCTGAGCA
CGCTGACACACAACACGGCACCGCAGTGGACGCCGTCAGTGAGCGAGGGGTCTTCGCTGGCGCAGCAGGTCGGCCATGTCATG
CACCACAGCACAGCCTTCATGCTGGGCTCAGCCAGCGCCACAGACCCTGAATCATCTCTCCTTCTGGACACCAATATGCAGGC
GGCGGCAGCTGTGTGGGATCCGAGCCAGGCGCATGGGCTGGACCAGAGCGGCTCTAAGCAGCTGTATGAGGAGCCAAAAGAGC
TGTACGAGGAGCCCGGACAGCTGTATGAGGAGAGGGGCGGCCACGTCAAGCCAGAGGAGCAACTCTGA
>Symbiochloris_reticulata.PSR1
ATGGACACCCATGACATAGCGCCTGCGCTTCCAGAGAGAAGCTTGGAGTGGCTGGAATTTTGGCCAGAGGCAGAGTTCAGAGC
AGAGGACCACAGTATGAACAGCAACCCGTTTTGTTGGGTACAGCATGATGATCAGCAACCGTCAAGTGGCGCCAAAGCCTCCG
CTGGACCAGTGTCAGCGGCACAGACCTCGGAACCTGGCATGCTCTTTCCAGGGCAATTGCAGCCGGTCTCTGCAGCCCTTCTG
AGTCACTTCACGGAGGTGCATACTGGCGTGCAGGCCGACGTCTCGCACAGCTTCCTACCGCCCGACTATATGCAGCCCCACTT
CTGTGGTAGTGATTTGCCGGAAGCTGCCCACTCTATGCCGCTTGCTTCAGCAGCTCAGCAGGCGCCTGTTTTTGGTGCAACTG
CCAACGAGCCAAGCTCAGCTGGAGCTGGCAGCTCACAAGCTGGAAAGCCGCGCTTGAGATGGACGCCAGAGCTGCACTCCCGC
TTTGTCGCTGCCGTCAATCACCTGGGTGGACCTGATCGAGCTACTCCCAAAGGCGTGCTCAAGCTGATGTTGGTGGAGGGTCT
CACTATCTACCATATCAAGAGCCACCTGCAGAAGTATCGTCTGAACATCCGCCTGCCAGGCGACTCCGGCCCCGTCGGCAGCC
TCAGTGGCTCACGCAAGAAGCGCAAACGCAGCAGACGAGCCAGGTCCTCAGACTTGGAGGATGAGGAGGAGGAAGATGACATG
GATGAGGCAGACAGCATGGAGGACATGTTGCCTGGAGATGAGCTACATGGCAGGCAGCAGGCAGTTGGTGAGGCTGGTCTGGC
ACTGGATGCAGCCCTGCCAGAGCAGGGCAACGCTCAGGTGCCTGGGCAGCAGCCTGAGCAGCAGCCAAATGCTCAACGGCAAC
GAGATCTTGAAGAGGCCCTCATCTTGCAGATGGATATGCAGAAGCGCTTGCACGAGCAGCTGGAGTCACAGCGGCAGCTGCAG
CTCAGCCTGGAGGCGCACGGCCGCTACATCAGCAGCTTGATAGAGCGCGAGGGTCTGCAGAGCAAACTGCCAGCCGGGACGCA
TGCAGCCATGCAGAGCGGCCTGCCCCGGCTGCCCGAGGCGTCCCTCGGCATGGCTGCAGGCATGTGCGGTCCAGCAGACGGCA
GCGGAGCGGGCACCATTGCGCCGGGCACGTCCGGCGGCATGTCATGGGGTCAGATGACCCATGTCACCCTACCGCACAGCGCA
GAGTCACCTCCGCTGCTGTCCCACACCAGCCGCACCGGCGCTACTGCTGCGGATGCCGGGCAGTTCCTGATGGTCGGGGATCC
CGGAGATCTGGGGCCGCTGCCCAGCATGCTTCTGGACACCGATCTGCAAGCAGCGGCAGCTGTGTGGGACGACGGCATGCACC
GGCCCCGAAAGCATGCGCCGAATGGGCACCTAGAGCATGCTTCGGGGCTTGACGAGGGCCTCTTTGACCAGCATGAAGGGGAA
GAGCATGGAAGGCTGCAGCGCCGCAGACAGCCATCTTCTCGCCTCAGACAATCATGA
>Edaphochlamys_debaryana.PSR1
ATGGCCAAGGCTGAAGGTCGCCCTGGAACCATTGTGGGAAGCGAGGAGGACTGGTTATTGGAGTTTTGGCCGGAGCCGACGCT
GGAGCTCTCGGGGCCGGCCGCGATGCAGCCGCAGCAGTCGCCGTCGCTTGACGCGCCCATAACCGACCTGCAGCAGCTTGCAC
CACAGCAAACATCACAACAGGCGCAGCAGCCCGGAGGGCTGCCGCTGGGGCAATACGCGCTCGCCTCGGCTGCCGATTACCTC
CAAACCGCGCAGCATGCCCTCAGCGCATACGACCCCTACCGGACCAAGTCCGCGCCGCCCCTGCCCCTGGGCCTGCTCCCAGA
CCGGCCTTCGGACTGTGCATCGGGGTTACTGCCTCCGCCAGCTGGCGGGGAATACCTGGGGGCCCTGGGCACAGCCCAGGGGG
GTCTGGGGCCCGTGCCACACCCCCTCATGGCGCCGGGCGCTGTCTCAGGGCTACAGCAAGGGCAGCCGGGGGGCGGCTACGGA
GACCTCGGCCTGGGGGCCATGGGCATGGGCATGGGCGGCCTGGGCCTGCAGCAGGGGATGCTGCACCCGCACGCCCATTACTT
CGCGGCGCCCCCCCGTGCCGCCGCGGGCCCTAGCAAGAGCCGGCTCCGATGGACGCCCGAGCTCCACAACCGCTTCGTCCAAG
CCGTGAACACGCTCGGCGGGCCTGACAAGGCAACGCCCAAGGGCATTCTAAAGCTCATGGGCGTGGACGGACTCACCATCTAT
CACATCAAGTCCCATCTGCAGAAATACCGCCTTAATATTAGGCTGCCTGGGGACTCCGCCGCCGGGCCCCAAGGCGACTCCGC
CGATGACTCCGACGCGGAGGGCGGCGGCGGCGGCACGACCGCGACCGGCATGGCGGCGGCACCGTCGATGTCGTTAGACCGCG
GCGGCATGGAGACGACGTCGGGGCTGCTGGGCCGTCGGCTCGGCAGCAACGCCGCCACCGCCGCCGCCGCCGCGGGCTTCCTG
GCGGGGGGCGGCGGGGGTGGCGGCGGCGGCATGGCGGAGCCAAGCCTGTCCAACTCGATAGCGGCGGCGCAGGTGGCGCAGCA
GCAGGCCGCCGCCGCGGCGGCGGCGCAGATGGCGGCGGCGCGGCCCGCCGGCGGCAGCACCAGCAGCGGCAGTACGCCGTCGG
CGACCCGGCGGAACCTGGAGGAGGCGCTGCTCTTCCAAATGGAGCTCCAGAAGAAGCTGCACGAGCAGCTGGAGACCCAGCGC
CAGCTGCAGCTCAGCCTCGAGGCGCACGGGCGCTACATCGCCTCGCTCATGGAGCAGGAGGGCCTCACCTCCCGCCTGCCGCA
GCTCAGCAGCGGCGACGGGCCGACGGCGCAGCTCGCGCTACCCGGGCCCGGCGGCGAGGGCGGCGGCGACGGGCTGCAGCGGC
AGCCGTCGGGCATTGGCGGCGGCGGCGGCGGGCCGCAGCAGGGCGGGCCGCTGGTGGGGGCGACGGGGCAAGGCGTGGACCAC
GCGGGGCTAGGCGGCGTGGGGCCCGACGGGCGGCGGATATCCTCCCAGGGCCTCGGCGCGCCCTCTCCCCAGGCGCTCCTGCC
GTTCCAGTTGTCCAGTGCTGGGCAGCCGACGGGCCGCCACCAGCTAGGGATGCAGCCCTCGCCCCAGCACCTGCCGGGGCCCG
GCGGCGACGGAGGCGGCGGCGGGGGGCCCGGCGACGAGCATCAGCGGCGGCGGTCCGAGATCGCGTACGACGGTACGGGTGGG
TCGGGCCTCACTGGCGGCGCCAGCGGCGGCTCGTCCGTACAGCAGCTGGCGGTGGCGGAGGCGCAGCGGCATGACCTCATGCG
AGCCGGCCGCCTCGGGTCCATGCCGTCCGCCGCCGCCGCCGCACTCCAGGCCGCCGGCTCCAACTCGCTTCCGCAGCAGCACA
TGTACTCGCCCGCGCAGCAAGACTCCCTCGGCCTCTCCCAACAGCAGCAGCAGCAGCAAGCACAAGCCGACGCCCAGGCGCAC
GCGCAAGCGCACGCAGCGGCCCAGGAGCACGCGGCGGCTGCCGCCGTCGCCGCCGGCATGCAGCTCAGCATGGCACACGCGCC
GTCGGGCAGCGGCCTCGGCGACGGCGGCGGCGGGCTGGGCGACGGCGGCGGCGGGCTGGGGGATTTCGACCTGGCGGACTTCG
TCGGCGACCTAGACGCCAGCGGCGTGGCGGCGCTCGAGGGGCAGGGGTTCGCGGGGCTCCAGGGGGGCCTGCAGGGGGACTCC
GAGATGGGCCTTCTTGCGGGTATCGGAGACGACCTGGCGGCGGCGGCCGCCGAGGCGCAGGCACAAGGGCTCGTCTCGCCGCG
GCGCGGGTCCTCGGGCGGGGAGGATAGCGGGCGGAGCAAGCGCGCGCGGCTGCAGGGCAGCTCGTCGGGGGAGGGCCAGGGCT
AG
>Enallax_costatus.PSR1
ATGGATCCCGGGCCTAATCATTCTTTGGGCCCTTTAGAACCAGATCATTGTGACCTTGGATACCTGGAGTTTTGGCAGGAAAG
CGAGTTCAAGCTCGAACCTGCAGCACACCATACCTTAGGGATGGACCACCTAGGCGACCACTTTTTGGCAGCAGGAGCAGTTG
GAGGGAGCGCAGGGCAGTATGGGCAGCTTGGTTTGGCGGCGGGACCAGACCCCTACAGCAACCAAGGCATACCTCTGGTGCCA
GCCCTTAACGATCAGCACTTCCAGGCGGGTGACGTATCGCTGCTGAGCACGTCCACGGGCCAAGGCAATCAGGTGCCACAGCT
GCTGACAACACCTGCTCTGGAGTCTTATACGTCGTCCTACGGGGCAGACCCACTCAGCAGCATGCCATCCGGGGCAATGCTTT
ACTCATCGGGAGCGTTCGCGATGCCTGGCAGCAAGGGGTCTTCAGCTTTTGACGCTCCTTCAAACAAGACACGTCTGCGCTGG
ACACCAGAGTTGCACAGCCGCTTTGTCAGCGCAGTGAATCAGTTGGGAGGTCCTGATAAAGCAACACCAAAAGGCATCCTTAA
GCTCATGGGGGTGGATGGGCTAACCATATTTCACATCAAGAGTCACCTGCAGAAGTACAGGCTCAACATCAGGCTGCCAGAAG
GTGCTCAGCCAGCCATGAGCACCGGGTCTATGCAGGAGGGGGATGCAGCAGCCGCAGCTGTAGATAGTGCAGCTGATACACAG
ACAGCGGTGATGTCCGGAGCACAAGCAGCTGCAGCTCAACAGCCAAGTCAGCAGCAGCAGCAGCGTGGTCAGCAGGATAAATC
TGGTCAGCAGGATAAGCCTACTCAGCAGCAGCAGCAGCAGGCACCAGCTCTGGTCCCTCAGCCAAGCAGCAGTGCTGGCCGTG
CAGCTGCTTCTCTATCCCCATTGATACGTGAGGGTTCAACAACATCTATACCTGGGCTCAGCTCAGGGGCAGTGCCCGACATG
CAAGCACCACTGCTACCTCCTGGTACTGGCTCGGGTGGGCCGGCAGGACAGCAGCAACAGCAGCAGTCGCAGCAGCAACCGCC
GCCGCCGCAGCAGCAGTTGAAGCAAGCGCAACAGCAGCCTCTACAACAGCCGCAGCAGCATGCTCGGCCGGTGCCGGAGACGG
CAGCAGCAGCTGGAGGAGCAGCAGCTACTGACGAGAACAATGATGCTGCCATCAAGCACAGCACTCGGCGTGACCTGGAGAGG
GCTCTCCTGCGCCAGATGGAGCTGCAGAAGCAGCTGCATGAACAACTTGAGATGCAGCGTGCACTGCAGCATAGCTTTGAGGT
TCATCAGCGCTACATACACAGCCTCATGGAGCAGGAGGGGCTGGCTCACAAGATACCAGAGATGTCAGCAGCCTTAGGGGCAG
TAGCAGCAGCCACAGCTACAGCACCACCTGGAAGTGTGGTCAGTGAAGCCATGCCAGTGCAGCCCGCACAGCCCAGTAACAAC
AGTCAGCCACTGCAGCAGCAGCAGCAGCAGCAGCCGCCAGGAGCACAAGCTGGAGCTGCAGTGCCAGCCCCTGGGCAGCAGCA
ACAGCAGCAGGTGCTGCAGCTACCACAGAAGCAACAACATACAGGCCATGCAAGCGCCAATGATGCTGCGCCTGTGGCAGCTG
CTGCGGTGACTGATCAGTTCCTGAGTGATGCTGAGCTGTTGATGGGTTTCCCTGATCTGCAGCATGACACCGGTGACATAGAC
CCCATACAGCAGCACCTGCTCGGGGATGAGGCAGCAGGGGGACCACCAAAGAGGCAGCGCATGTCAGGGCAGGATGTCTGA
>Mesostigma_viride.PSR1
ATGAACCGCCCCCCGGTCGGGAGTGCTTCACGTACCGCTGACCAGCAGCTGAGTTCGTCAGCGGAGGTGCAGCCAAGGACCGT
TCAAAAGCTGCCTACCACTGTTGACGAGCTTATAAATCAAGAATGGCCTATTTGGGGAGAGCTTGCGCCTAACGATGACAGCA
TTACTACTTGCTGGACGGACTTGTTGACCGGGCCTCCGCCCAAGAATCAAGACATGCACCGTCCACAACATGCAACCATTCAA
GATGATACGTCTCCAGGGCTTTACCTTGCCAGGCAGCAGTATCTCCCAGGGATGGGGACACTGCCACCTGGCGGGGTTCCCCC
CCTGTGTGCCCCCCCCGGCTTGATGGATGGTGGCGGCATGAACCTGGTGCCGGGCATGCAGGCCTCGATGGCGGCGGCCCAGT
CGCAGCAGCCGCCCAAGCAGCGGTTGCGGTGGACGCCAGAGCTGCACGACCGCTTTGTCAACGCGGTGCAGAACCTGGGGGGA
GCGGACCGTGCCACTCCCAAGGGTGTCCTGCGCGTGATGGGTGTGCAGGGGCTGACCATCTACCACGTGAAGAGCCACCTACA
GAAGTACCGGCTGGCCAAGTTCCTGCCTGAGGAGGGAGGCAACTCATCCAAATCGCTGGGCGGTAGCAAGCGCGACACGGACA
GCGACAACGATGACGCGTCGGACGGCGACCCGCTCAAGATGGCGGACCTCAAGGCGGGCGCGACCGAGCTGCTGACGGGGGAG
GACGGCTCGGTGAACATTGAGGAGGCGCTGCGCATGCAGATGGAGGTGCAGAAGCGGCTGCATGAACAGCTCGAGCTGCAACG
CGCGCTGCAGCTCAAGATCGAGGCACAGGGTCGCTACCTGCAGCAGATTATGGAGGAGCAGCGGAACGCCGCGCTCGCACGCC
GCGCGCAGGCTGGGGGGGCGGCCAGCGGCGCCACCACGCAGGGGCAGGCCACCTCGGCAGGGCAAGCTGCGTCCGCCAGCAGT
AGCAGGAGCAGCGCGGGGGGTGGGGGAAAGGGGCCCGAGGGGGCAGCAGCGCCGGCAGCGGGTGAGGGAGGTGCCGGGGCGGA
TAGCATTAGCCACGCTGCGGATGGTGCCCGCGCGGGTGCAGAGGCAGGAGATGCACAGCGCCAGGCGGTCGCCTCCCCATCGG
GCGCCCCCGTGTTTGCAGCCTCGGGCGTACACGACGCGGATGGAGCGGGCGCCACCTGTCCAGCCGTGGGCGCAGCAGGTGGC
CACGCGCCATCCCCGGCCCTGGTGCCGAAGACAGAATCGGTTGCTTGCGGTGGTTCCCTTGCGATGCCCGATGCGCTGGCGTC
ACTCCCAGGGGGCGGCGGTCACCACCTGGGCGCGAGTGGCAAGCTGCCAGGGTGCGAATTGCCGCTGCCCTCGTGGTCGGAGC
CAGGTGCCGCGCTGCTGACGGCCAACGGGGGCATCTTGCCGTTCCCTAGTAAGGTCGAGGGGCGCAATCTACCCCAGTTGTCG
CTGCCGTCGCACCTGCTGCTGGGGGTGGAGGATGTGGATGATGGTGGCGGTGGTGGGGGTGGTGGTGGCGGTGGGGGCCAGTC
CTTGCAATCAGGTGTCGGTGTTGGCAGCAAGCGCGCGTACGATGAGATGATGGGGGGGGGCATCGCGATGGAGGACGGAGCGG
GCGACCGGCTGCACACGGACGGCAGTGGGTTGCCCACAGGGGGGTCCCTGCTGCCCGATGACGCGTCCCTGCTGGCGGCCCAG
GGGGGGCACGCGTCCGGTGGGGCGGACCCAGCTCCACACATGCTCTAG
>Raphidocelis_subcapitata.PSR1
ATGGCGGAGCGCacccccggcagccccgcgggggagggcgacgaggcggTTCTCGCGGGCCTCGCGGGCTGGCTGAACGATGA
GCTCAGCTATTGGCCGGAGTGGCCCGtcggccccccagccccgcccctggACCCCCAGGCCCACTGCGACGGCCCCGTGATCG
CTCTCCCGGGCGCTCACTGCCCGatggagcagcggcaggttgCAGCGGGACCCCCCGGACCCCacgggggggcgggaccCCAC
GCGGTGgcacagccccagcagcagcatcccGCCCTGCAGGCGGGCCAGGGGCACGCCTTGGATGCTTTCCAGTCCTACCAgGC
GACCGCTTACGGCATGCAGTTGGCggtgcacgcgcagcaggggggctTCGACCCTGGCatgctcggcgcggcgggcgcgctcg
cgcccggcgcgctcttCGGCGTGCCGCCAGCGTACGGCatggccggcggcaagccaGgcgccatgGCCGGCGGCAACAAATCC
CGCCTGCGCTGGACGCCCGAGCTGCACGCGTCCTTtgttgccgccgcggagTCGCTCGGGGGCGCTGACAAGGCGACGCCCAA
GGGCATCCTGAAGCTCATGGCCGTGCCCGGGCTCACCATCTTCCACATCAAGAGCCACCTCCAAAAGTACCGCCTCAACGTGC
GCGCACCGGACGGGACCGAGggcgccagcgacggcggcggggagtcggccgtcgagggcgccagcggcgagggcggcgcgacg
gtgcgcatgggcgcgctgcgggcggagaGCCTGGATGCCACGGCGCCtagcagcgcgctggcgctgccgccgacggcgctggg
cgcttCGCCGGCGGTGGGTGTGAAGCCGGAGCACCCAGAGGTCGATGCGCACAGCCTGCttaagcagcagcagcacgcagtGC
CTgccagcaccaccagcacgTGCGCGGGCCTCAGCAGCGCGAcgggcctcgaggcggcggcggcggcgggtggcgccggctcc
gaggcggcggcgggcggcccctccacggcgcggcgcaggaacCTGGAGgatgcgctgcagctgcagatGGACCTGCAGAGGCG
GCTGCACGaccagctcgaggcgcagcgggcgctgcagctgagcCTGGAGGCACACGGGCGCTACATCGCGCGGCTGATGGAGC
AGGAGGGCCTCGGCCACAGGCTGCAGGACCTCGCGGCGATaaccgcgccgggcccgggcgcgggcgcggaggccgaggcggcg
ccgggcggcggcgatggcggcggcgcggcgggctctggcggcgcgggctctggcggcgcgggccccggcggcgcgcccgcggc
ggcgccggccagcgaggcgaacagcagcggcttgagggcggcggccggcggctgcggcggcggcaggagtgtggcgggcggct
gctgcgacgGTGCGctcccgctggcgcgggcgggctccTCTGCCCTGGACAGCAGCGACCACCCCGCAgagccgcaccagcag
ccggcgaggtggcagcagcccacgccgccgccctccgcgtcagggcggcgggacgaCCGCAGCCAAGACCagcggctgcacgc
cgccgccgggcagctgctggcgtggggccgcagcgcgccgccgccgcacgacgcggccgggctggacgcggccggcgcgccgc
agggtaagcggccgcggctcagTGGCGCCTGA
>Symbiochloris_reticulata_Africa.PSR1
ATGCTCTTTCCAGGGCAATTGCAGCCGGTCTCTGCAGCCCTTCTGAGTCACTTCACGGAGGCCGACGTCTCGCACAGCTTCCT
ACCGCCCGACTATATGCAGCCCCACTTCTGTGGTAGTGATTTGCCGGAAGCTGCCCACTCTATGCCGCTTGCTTCAGCAGCTC
AGCAGGCGCCTGTTTTTGGTGCAACTGCCAACGAGCCAAGCTCAGCTGGAGCTGGCAGCTCACAAGCTGGAAAGCCGCGCTTG
AGATGGACGCCAGAGCTGCACTCCCGCTTTGTCGCTGCCGTCAATCACCTGGGTGGACCTGATCGAGCTACTCCCAAAGGCGT
GCTCAAGCTGATGTTGGTGGAGGGTCTCACTATCTACCATATCAAGAGCCACCTGCAGAAGTATCGTCTGAACATCCGCCTGC
CAGGCGACTCCGGCCCCGTCGGCAGCCTCAGTGGCTCACGCAAGAAGCGCAAACGCAGCAGACGAGCCAGGTCCTCAGACTTG
GAGGATGAGGAGGAGGAAGATGACATGGATGAGGCAGACAGCATGGAGGACATGTTGCCTGGAGATGAGCTACATGGCAGGCA
GCAGGCAGTTGGTGAGGCTGGTCTGGCACTGGATGCAGCCCTGCCAGAGCAGGGCAACGCTCAGGTGCCTGGGCAGCAGCCTG
AGCAGCAGCCAAATGCTCAACGGCAACGAGATCTTGAAGAGGCCCTCATCTTGCAGATGGATATGCAGAAGCGCTTGCACGAG
CAGCTGGAGTCACAGCGGCAGCTGCAGCTCAGCCTGGAGGCGCACGGCCGCTACATCAGCAGCTTGATAGAGCGCGAGGGTCT
GCAGAGCAAACTGCCAGCCGGGACGCATGCAGCCATGCAGAGCGGCCTGCCCCGGCTGCCCGAGGCGTCCCTCGGCATGGCTG
CAGGCATGTGCGGTCCAGCAGACGGCAGCGGAGCGGGCACCATTGCGCCGGGCACGTCCGGCGGCATGTCATGGGGTCAGATG
ACCCATGTCACCCTACCGCACAGCGCAGAGTCACCTCCGCTGCTGTCCCACACCAGCCGCACCGGCGCCACTGCTGCGGATGC
CGGGCAGTTCCTGATGGTCGGGGATCCCGGAGATCTGGGGCCGCTGCCCAGCATGCTTCTGGACACCGATCTGCAAGCAGCGG
CAGCTGTGTGGGACGACGGCATGCACCGGCCCCGAAAGCATGCGCCGAATGGGCACCTAGAGCATGCTTCGGGGCTTGACGAG
GGCCTCTTTGACCAGCATGAAGGGGAAGAGCATGGAAGGCTGCAGCGCCGCAGACAGCCATCTTCTCGCCTCAGACAATCATG
A
>Tetradesmus_deserticola.PSR1
ATGGACTCTGGTGCTCATGACTTAGGGGACCATACAGGCGATTGGCTTGAGTTTTGGCACGAGTCTGAGTTTAAGTTAGACGG
CGTATCGACCGCAGCAGCCCAGCCCGGCCAGCACGCCCCTATGGACCTGCCTGGAGGGCTCGGCGACTTCTTCTTGCCCAGCG
GCAGCATGCTGCCGCAGCCGCACTCTGGAGACGCGCAGCAGTTGGTATTAGCACCTGCAGGCGATCCCTATGCAGGCAGCCTG
ACCATGCTGCCAGGGCTGGAACAGCAACAGCAGCACTACAAGGGGCCTGACCTGTCGTTCATGAGCACATCCTCTGGAGCAGC
AGGGCAGATGACGCAGTTGATGCCGCCTACTGCACAGCTGGAGTCGTACACTTCTTCATTCAGCTCAGACCCTACCCTCAGCG
GCATGCATTCAGCACCTATGCTGTATCACGCAGCTTCTTTTCAGCTGCCGGGCACGAGGTCTGGGAGCCTGCAAGAAGCCCCT
GCAGGCAAGACACGGCTGCGCTGGACACCGGAGCTACACAGCCGCTTTGTGCAGTCAGTCAATTCCCTTGGCGGCCCTGATAA
GGCAACACCTAAGGGCATACTGAAGCTCATGTCAGTAGATGGGCTCACCATCTTCCACATCAAGAGTCACCTGCAGAAGTACC
GTCTGAACATCAGGCTGCCGGAGACCTCAGAGATGGGTGCACAGCCTGCAAACAGCAGCGGATCACCAGACCAGGAGGCAACA
GCAGCAACAGACAGCGCAGCAGACACGCACGCAACGCTGGCGACAAGCACCATAAACCCATCAGCAGCAGCAGCAGTGGCAGC
AGGCGCTGCCGCACCTACTGCAGCTGTGGCACCAGCCAGTGCGAGTGCTGGCGGGGGTAGTTCGCTGCAGCAGCAGCAGCAGC
AGCAGTCACTGGTTCCTACGTCTCAGCAGCAGCAGCAACAGCAGCCACCGCCGCCGCAGCAGCAGCAGCAGCAGCGCATTTTG
AGTGGTGTCGAGCAGCTGTCAGGTGCATCGCCACTGCAGCTGACCACTTCAGGCGTTCTGGAGATGCCAGACAGCGCTGCGTC
CGCAGCCCAGCAGCAGCAGCAGCAGCAGCAGCAGCAGCAACAGCAGCCCACAGGCGCTGCAGCAGATGCTGCAGAGGACAGCC
TGCACATGAAGAGTGACACGCGGCGGGACTTGGAACGTGCACTGCTGCAGCAGATGCACCTGCAGAAGAAGCTGCACGAGCAG
CTTGAGACTCAGCGTCAGCTGCAGCATAGTCTGGAGGTCCACCAGCGATACATCCACAAGCTCATGGAGCAAGAAGGGCTAGC
GCACAAGATCCCTGAGATGTCAGCAGCTTTCAACGCGGGAGCATTGCCACCGCCAGGCAGTGTCGTGAGTGAAGCAATGCCAG
GCCAGCCACTGGCAGTCGGAACCGCTCCTCAGCAGCAGCAGCAGCAGCAGCAGCAGGCGAGCAGTGCAGCGCCGCCGCTGCAG
CGGCACCATTCGCTGCCGCACCAGCAACAGTTGCACACTGGTGTCGGCAACTCTGATGCAGCTGCTGGTGTTGGCACCTCAAA
GCGTAGCAGTAGTCACCACCACCACCACCACCATCAACATCATCAGCAGCACCACCCCCAGCAGCAGCACCACCCGCAGCCGA
TGCAGCAGCAGCAGGAGCCAGGTCAAGACGCAGCAGGTATCGACCCGCTGCCTGGCAGCTGTGGCAACCTGCTGAGTGATCAT
GAGCTGCTGCTGGGGTTCCCTGAGCTGCGCGACAGTGGTGACGAGGGCGGGGGCATGGGGCTGCTCAGCGAGCCTGGGCAGCC
GCAAGGCAAGCGGCAGCGGCTGCTGACGCCCGACATAGCCAAGTGGCCTTCAGTGGACAGCGCTGAAGGCCAGCACTGA
>Tetraselmis_striata.PSR1
ATGAACATTCGTCACGACGACGATGCCGCCGCCGCCAGCGTACAGGTGCGAGAACGGGTTCCATGGTCACAGGGGATTCGGGG
GGTGGAGGGTGCGGTGGTGGTGGTTTTCATCCTAGCCCGCTTCACTCCAACCGCGCCACAGACCCGCCAGACCTTGCAGCCCC
CGTATCGCCAGCTCGCGCGGCATCGTGAGGACCCTGCCACGAGCCCCGACCCGCGCACCGGTCGTCCCGCCATGGACCTCAAC
GAGGACGCGGACGCGGAGCTGAACTTTTTCAAGGCGATGGAAGCCTTCAGTCCACCCAGCTTTGAGGGCGGCGAGGCGGAGGA
CCACCTGCACGGCCTCAGCGTCCCGGGTCTCCCCCACATGGCCGGGGCCACCGATACACATGCGCACAATCCCCCCGCCAGCA
CAGGAGACGGCTCGCAAAGCGCCACGGTGCACAACACATCCGGGGCACGTGGGCATGACCTTCTGGTGAACAACGGGCACAGC
ATGTGGGAGCCGCTGTCGTTTGAGGAGGTGATGCGCAACGGCGGCGTGAATCCCTCCCAGGCCTCAAGCCTCGCGTCGACCAG
CACCGCCGCCACGGAGCTGCTGATGCATCGCGGCAACACGTTCCTGCCCAGCGGGAATGGCGGCGGCAGGCAGGCGCCGCCTG
GCCAGTTTGGGATGGGCGGCATGCCGTCCATGATGGCGTTTGGTGCCCCGCAGCAGCAGCAGCAACATCAGCAACACCAGCCG
ACGCCTCAGCAGCAGCCGCCGCAGCGGAATGGGTCGGAGGATGGCATGCAGCACTTTGGAGGCCTGTTCCCGCAGTCGGCGGC
GTTCCGGCCGCGGCTGCGCTGGACCAACGACCTGCACAACCAGTTTCTGGACAGCGTCGAGCGGCTGGGCGGCACCGACAAGG
CCACGCCGTCCGCGATCCTCAAGCACATGGGCGTGGATGGGCTCAGCCTGGGCCACGTGAAGAGCCACCTGCAGAAGTACCGC
ACCGAGCTGAAGCGCGCCAAGGCGGTGCGGGGCAAGGCGATGGACGACATGCACCAGATGAAGAAGGGGGCGCGCAGCAAGGC
GGCGGCGGCGGACGTGGCGGCGGAGGCGGCGGAGGTGGTGGCGGAGGCGAGCGGTAGCGCGGAGGCGGGGCTGGAGCAGCTGG
GCGCGACGCAGCGGGAGCTGCAGCGGCAGCTCGCGGCGCGCGCGGCGAGCGGGCCCAACGCCAAGGAGCTGGAGGAGGCGATG
CGCACGCAGCTGGAGCTGCAGAAGATGCTGTGCGCGCAGCTGGAGGCGCAGAAGGAGCTGCAGCGGAGCCTGGAGCAGCACAC
CAAGTACATTTCTGTGCTGATGAAGCGGCAATCGGGGGACGACCTTCACGCGCACGGCGAGGGTGACACGGCCGGCGAGCATG
AGATGTCCAAGGCCTGA
>Trebouxia_sp..PSR1
ATGGACAACGACACCATAGACTGGCTTGACCTGGACTACTGGCCTGAGAAAGATTCCAAAAAGCCTGCAGACATGGACAACTC
GTTCGCTTGGCTTGCACAGCAAGCTCAGCCATTATCTGGGCAGCCACTGCCAGGATCGCAGTACCAGGTCCAGCCACATGTAA
TGCAGCCACATGATGGACTGTTGTATCACGACACGTTTCACCCCCACTCCACAGCAGGATCCCTGCTTTCAGACCTCTCAGGG
GATTTGCTAGATACAACAGCTGTAGACATTTCGAATGCACAGTTCGAGGCCATTCCTTCGCAGAGCCCGCACCAGCAGAGCAA
CATGCAATTACGATCAGATACTGCTCACAACGGAGCCCCACAACCTCTGCAAGACATGATCCAAGCTCCCGTCTTTGGCAGAA
GCACTTCATCTATGTCTCAGCAAGCAGGCAACAACTCCCAGTCTGCAGCAGCACAAGCTGCTGGCAAACCGCGCTTGCGGTGG
ACGCCTGAGCTTCATACTCGCTTTGTTGGCTGTGTAAGTCAGCTAGGCGGTCCTGAAAAAGCCACACCCAAGGGTATCATGAA
GCTCATGTCAGTGGAAGGTCTCACCATATACCATATCAAGAGTCACTTGCAAAAGTACAGGCTAAACATCAGGCTGCCGGAGT
CAGAGCAGGTCGAAATGAGTGAAGCTGTGTCAGGTGAGCATGAGGGGCGCAAAAGTCAGCGAGGCAAAAGGCGCAGCACCAGG
AAACAGCGCAAACGCTCAAAGCGCTCGTCTAGCAGGCGACGTGCCTTGGAGAAGAGTGATGGTGATGATGATGAAGCCGATGA
CTTGGATGATGATCAGTTCGATGAGGAGGAGGGTGACAATGAGCTGGATGGGCATGCTGCTTCTTCTGGAGTAGGGGAGGCTT
CCAGCATGCTGGATGGAGTCACCAACAGGGAAGAAGATGCACAACGTGAGGTGCAGCGGCAGCGCAATCTGGAGCAGGCTTTG
CTGATCCAGATGGAAATGCAGAAGAAGCTGCATGAGCAGTTGGAATCACAGCGGCAGCTTCAGCTCAGCTTAGAGGCGCATGG
CCGCTACATCACCAGCCTCATTGAAAGAGAAGGTCTGCAGCACAGATTGTTGCCGCAGCTGGTAGCTGCAGCTGCCCCCAGTC
TGGCACGCACTGTCCCTGCCCTAGCAGCACTAGCAGCCTCAATGCCTCCAGGCTCCTCAGGTCAGATTTCAGATCAGCAGACT
CACTATATGCCTCTGTCAGCTTCTGGCGCTTCAGAATTCTCCCCTCAGCAGCTGCTGGCTGGCAGATTTTCCTCCTTGCCTAA
CTCAGTCAATCTGAACCAGGATCCCAGTCCCGGTGCAACAGACGCTGCAAGATCCCTGGACGTGTCGCCATCTTCGCTGAGCA
GGCATGTCAGTGGTGCAGTTCCGCGTAACCCATTCGGCACTATCAATCAGGCTGCGTTTGGAGAGCCAAGCTCGCCTGGATTA
CTGCTGAACACCGACCTGCAGGCTGCTGCTGCCGCTTGGGACGATCAGCAGCGGCATATCCTGACAGGTCCTGGAAGCAGACC
CTTGGATGGGATGCCAGCTGTACCTGGTCAATAG
>Chlamydomonas_reinhardtii.PTC1
MKFTHQLKFNSVPEWREHYIQYGHLKKYIYALAKKEADLQAGGQDEEALLAPLLEAERDQGPTEEGFQRELDAQLAATLSFFA
VKEADLLAKVSALELDIQSLEKIPNRAEASTLARMGGSASPGGPMSSPRAAAAAAMSAMASLVSHSPSTLDLARMVNSTPPED
HRKIRVKFWENPPRHLFSTNLNTRRAKLQARFQDLYISLHDLREFLHINKEGFRKIIKKHDKLTRAVDLRARWWPNVEAHLAP
AAKQAELDGAIGALTDHYAVLYTRGDVAQAEEQLSRGLREHITVERNTVWRDMAAMERKYAAVSVKQAAAPGARVTWLRTHAR
WLKLALSVAVFVVLANVEVWPGAENEPRNNCLALLVFASLLWSLEAVPLFVTSMALPLLIVALGVLVDRSKDPPQRMTPQQAA
PAIFHAMFSQTIMLLLGGFAIAAALSKHAIAKQVAVSILSRVGRKPRNVLLAAMFTATFASMWISNVAAPVLCFGLIQPILRT
LDPGHPFAKALVMGIALASNVGGMTSPISSPQNIFAIERMSLDGRPPSWLAWFAVALPVAVACNFVCWGLLLLCYQPGKAIAE
VRPIKPNTDPINGTQVYIIVVSLLTVAAWCANTFLQRYTGEMGVIAVVPLVAFFGFDVLNKDDENSFLWNVVMLAMGGLSLGE
AVKSSGLLAALALTISDLVMGLSLWQVAAIFCGMVLVATTFISHTVGAMVILPIVQSVGEAMAGTPHPKLLVMAAALMCSGAM
GLPVSGFPNMNAVSLEDSTGNAIVGTGDFLAVGVPSSVFAYGIIVSLGYVLMLAVGF
>Monoraphidium_neglectum.PTC1
MDKAERELRSELREEVGFERNTVWRDMVAMERRTGAVVRQDTHGITDDTIREPWVKRYWQPMTLTVSLIALVTLLLVPIFEDE
PEKQNCLALLVFASLLWCTEALPLFVTSMIVPLLVVVLRVLVDRTVSPPERLSPEKAAPAVFHIMFGQVIMLLLGGFAIAAAL
SKHFIAKQLAVAILSRVGRRPRDVLLANMLVATFASMWISNVAAPVLCFSLVQPILRTLPPTHPFAKALVIGIALASNLGGMT
SPISSPQNIFAIERMSIGGDPPSWLTWFAVALPVAFFGNVLCWGLILIVYKPGLKIKEVRPLKPPEDPLSATQIYVVVVSLAT
VALWCCNNLLSHITGEMGVLAILPLVAFFGFGVLSKDDFNGFLWNVVMLAMGGLALGEAVKSSGLLLTIAQAIQSMVDGLDLW
SVLAIFCALVLMATTFISHTVGAMVILPIVQSVGQQMPGHHDKLLVMGAALMCSGAMGLPVSGFPNMNAVALEDPTGVNYVDT
VDFLKVGVPGSVMVYWLIVSVGYVLMRAVGY
>Entransia_fimbriata.PTC1
MKFSHALKFNSVSEWKAFYIDYVHLKRFVYQLEAAAVTALPETPRLTSLSLVSTLGDVEAGEEASGPSPLVTDAAFVHALERE
LEKIVTFYAKKEQELVSQIENAETQAREFEARQYTRSRRQGHGQSTMTAPEGGRRVGDEGAVDLREPLLGGKESSGLGSGAGP
DSTAQRVWVWQQEDSGVRLAKERLRLQMTDLYVQLFGLQDYIDLNRTGFRKILKKHAKVTHHALQSEFMPRVNVGLDKKHEQQ
QEAALARVVQLYSIVCCNGSSELARLELKRHLRDHLVWERNTVWQDMVSKERRSASAHVYEEKTAWYLRCLRPLSLLLAVGVL
VALLVVPVFPEAPKQNCLALLAFVSLLWCTETIPLYTTSMLVPLLAVVLRVLVDSKTGRRLSPQEAASAIFHTMFSEVIMLLL
GGFAIASALSKYFIAKRLATTILSRVGQAPQNVLLASMLVASFLSMWISNVAAPVLCFSLVQPILRTLPSGHPYGRALVMGIA
LASNLGGMASPISSPQNIFAIEELSLLGDPPSWLQWFVIALPICLVGNIGCWALLLAAYAPHKQPNTIRPLKPTQDPITLSQV
YVVVVSVVTVALWCANSWLRQYLGQMGVVAVVPLVAFFGTGILTKDDFNNFLWNVVMLAMGGLALGECVTSSGLLHSIAASIQ
ATVANLGPWQVAAVFCGLVLVATTFISHTVGAMVILPIVMTVGSAMPDPHPKLLVMASVLMCSGAMGLPVSGFPNMNAIALED
GTGKTYLTTLDFLWVGIPSSLLAYGIIITLGYGIM
>Golenkinia_longispicula.PTC1
MKFTHQLKFNCAPEWKDYYIHYSVLKKLIYQIGGDDIRESAGIGPAEQEPLLPVIDKDEKERQFESLLNTELTRILDFYTRTE
RELFSQLEGLGLEIREYEEGRLPTTSEGLETELDGRRRFWSSHDLPKDLKAAKDSLAMKLEDLYEELNNLLEYITLNHTGFRK
ILKKHDKVTQRELKSQYMPLVEAKLVHNKKERVQERIDQVVHQYAVIVCNGHEGRALTELRKKLRDHLVFERQTVWQDMVAIE
RRTAAAQVSNKAPEGGKGPRKWCQRLWSPLAIGASLLVLALLLRMSLFDEPAKQNCFAMLVFCSMLWCSEAVPLFVTSMMVPF
LVVVLRVLTDGTPERQRQTPNQAAGAIFHAMFSQTIMLLLGGFTIAAALSKHFIAKQLAAAILSRVGRKPATVLLTSMMVATF
ASMWISNVAAPVLCFSLMQTILRTLSPSHPFAKSLVLGIALASNIGGMTSPIASPQNIFAVERMSLHGSPPGWLSWFGVAIPV
SILSNLLVWAVILVVYRPGRYIKEVRPLKAPEDPLTGTQMYVIGVSLLTVVLWCCNSFLQKYTGEMGVLALLPMIAFFGFGIL
NKDDFNGFLWNVVMLAMGGLALGEAVKSSGLLASIAQGIEELVQGMSLYQVSVVFGLMVLVCTTFISHTVGAMVILPIVQSVG
ERMSDPHPNVLVMSAALLCSCAMGLPV
>Oedogonium_cardiacum.PTC1
EAIPLFVTSMLVPVLAVSLRVIVVDGRRLEPPDAASYLFGKMFSQVVMLLLGGFAIAAALSKHNIARKMAIAVLSRVGREPAH
VLLATMMVATFLSMWISNVAAPVLCFSLVQPILRTLDTNHSFAKALVMGIALASNVGGMASPISSPQNIFAIQVMGSGGDKPP
SWTQWFAVALPVSVVCNMLIWALLLLVYQPHKHIKEVRPIRAIQDKYTLQQVMVVLVSLVTVALWCLNGVLEPYLGSMGVIAI
LPLVAFFGFGILTKDDFNAFLWHVVMLAMGGLAVGECVKSSHLLQTIAAEIQEMTVGWSMYAIVCMFCALVLCCTTFISHTVG
AFVILPVLKSVGDEMARSGQPNHSKLLVMAAALMCSGAMGLPVSGFPNMNAVALEDQAGLNYVATIDFIKAGLLSSVFAYVII
ITLGYSLMFMIGF
>Cylindrocapsa_geminella.PTC1
MKYGTQLKLNMNPDWKDHYINYSMLKHMIYQISNKENQVPIDFEQGEPAEPLQPLLRWNSAPDLESTFVKAFEGELARVIEFY
TQKEAELFAKCGTLGLKIHQMDGLSAPPSDSPVASQAEDERIAFWAHVPGHLVAQRDELRKEMEALYVALKDLESFRSLNFEG
FRKALKKHDKETTTALMPQLMPVLQSKLSSSQTAIIQQRGDVVLHLFAVVVCHGDLQAARRDLDSRLQDEVLFERRSIWQDMV
ATERLRGSAPQVVPKDSGSAYGASLTGWFERNKQWTLLLLSFGAFAVLLNYPVEDDESKNNCLAMFVLLSLLWSTEALPLFVT
SMLVPLLVVILRVLVDNTQDPPVRLTPQDAAASIFHSMFSQTILLLLGGFTIAAALTKHFLAKQLAVTILSRVGRKPEHVLLA
NMLVATFLSMWISNVASPVLCFSLVTPILRTLPTHHPFGKALVMGIALASNLGGMTSPISSPQNLFAIERMGLDGHPPSWLAW
FLVALPVSFLGNLICWGVLLAVYRPQQKIREVRQLKPSGDPITWKQVYVLVVSLTTVGLWCANTALQQYTGEMGVLAIVPLVA
FFGFDILNKDDFNHFLWNVVMLAMGGLALGEAVKSSGLLLTIAEAIKELVIGFDLWTVLVIFCGLVLVGTTFISHTVGAMVIL
PIVQSVGDQLPGPPHAKLLVMGSALMCSGAMGLPVSGFPNMNAVALEDPTGLNYVATIDFLKVGVVCSFFTYGIIVTLGYGLM
LLAGF
>Scherffelia_dubia.PTC1
LNVTGFRKILKKHDKVTNKELKGDFLPIVASKLNPKKERVDQVLQELIGVYATIGCEGDVNAAAAQLQVNLRDQVEFERQAVW
KDKIEEERKLANAKVVDKGKKAWYADYKKPFCLLLSAALIFGVLGSPLFPSSPPKRNCLAIFVGAAALWCTEAVPLYVTSMLI
PAAVVTLRALEDADGVRMTATETADRVFSKMFSQTIMLLLGGFTMAAAITKHLIAKRMAFQVLCRVGRRPGNVLLASMFIALF
SSMWISNVASPVMCYGIVQPILRTLAPDDRFASALVMAIALAANVGGMTSPIASPQNIFAVERMAMDGNPPSWLAWFSVSMPV
SMFCILVLWRIILAVYRIGPHTAEVRPMRPLDDVFTMQHVFIILISVLTMGLWCANTWLLSVLGGMGITALLPMVAFFGSGIL
NKLDFESFPWSVVMLAMGGIVLGEAVKSSGLLGFIAQGIVGLVDGFTVWQVLMIFGAVIGVVTSFISHTVGAMVILPVVQSVG
EEMAKASGVAHYKLLVMGAALCCSGGMALPVSSFPNMTAASLTDPT
>Hafniomonas_reticulata.PTC1
TAGNAEKAEGELSAHLREQVQYERNTVWRDMVANERTKTAESSKQVRTLSQSIVCIAIVAVVALIHANIFPDQPSKNNCLAML
VFVSILWASEAVPLFATSMMVPLLTVLLGIWVDPSQKPPKRLDHTAAASAVFSAMFNQVIMLLLGGFAIAAALSKHFIAKRVA
VAILSRVGRKPHNIILASMCVAAFMSMWISNVAAPVLCFSLIAPILRTLDASHPFAKALVMGIALASNVGGMTSPISSPQNLE
AIERMSMPPSWLSWFAVALPVAGISIILCWLIILIVYRPWTKVKEVRPLKQDDPITYTQAFVVLVSVVTVGLWCAESQLESVF
GQMGVIAILPMLAFFATGILNKDDFNGFLWNVVMLAMGGLVLGGAVKNCGLLEDIANAIKNQVADLELFQVLVVFCLLVLFCT
TFISHTVGAIVILPIVQQVGESFPGTPHSKLLVMGAALMCSGAMGLPVSGFPNMNAVALEDATGQTYVQTLDFLKVSVPCSIC
A
>Tetraselmis_chui.PTC1
LLSAAVLWCTEAVPLYVTSMALIFAVVVLRAMLDGDGVRMSAQDTMKRVFSKIFSQTVMLLLGGFTMAAALSKHLIAKRLAIG
VMSQVGRKPSNVLIASMAIALFSSMWISNVAAPVLCFSIVQPILRTLPTDDPLGAALVMGIAMSSNIGGMTSPIASPQNIFAI
ERMSLDGHPPSWITWFAVSMPVSITCMLLVWRLLLAIYPVSSNQDVRPLRQLHDPFTFQHVYIIIVCL
>Volvox_globator.PTC1
LWSLEAIPLFVTSMALPLLIVVLGVLQDSPNTEKPATRLTPQQAATAIFHAMFSQTIMLLLGGFAIAAALSKHAIAKQVAVAI
LSRVGRKPRNVLLAAMFTATFASMWISNVAAPVLCFGLVQPILRTLDPGHQFAKSLVMGIALASNVGGMTSPISSPQNIFAIE
RMSLDGQPPSWLSWFTVALPVSIAANMLCWAILLVVYQPDRAIAEVRPIKPNTDPTNGTQVYIIVVALLTVSAWCANTFLQSY
TGEMGIIAILPLVAFFGFDVLNKDDFNSFLWNVVMLAMGGLALGEAVKSSGLLAALASDISDLVLGLSLWQVTLIFCGLVLVA
TTFISHTVGAMVILPIVQSVGERMIGTPHPKLLVMATALTCSGAMGLPVSGFPNMNAVSLEDATGNPIVSTKDFLMVGVPSSI
AAYGIIVTLGYGLMLLVGF
>Volvox_aureus-M1028.PTC1
MKFTHQLKFNSVPEWREHYIQYAHLKKYIYALAKREADLQAGGQLHDDELLTPLVPETSRQGFSEEGFQRELDAQLASILSFF
AVKEAELLAKVSELELDVQSLEKIPNRQEASSMSRISGNPSTTGYHSSSSPRGPVGLPSMSLMSVSPSTLDLARMVDSTPPED
FRKVRVKFWENPPRHVFSPNLNTRRQKLLGRFQDLFIGLHDLREYLHINKEGFRKTIKKHDKLTRSVDLRVRWWPNVEVHLAP
VAKQQELERAIAALTDHYAVLYMGGDLTKADEQLSRGLREHITVERNTVWRDMAAMERKYAAVSVKQATAPRDGGRQAHVRWA
KLAACCLVFASLLLWGGPVENGQVNQPRNNCLALLVFASLLWSLEAVPLYVTSMALPFLIVAMGVLVDHPSDSKDPPRRLTPQ
QAAPAIFHAMFSQTIMLLLGGFSIAAALSKHAIAKQVAVAILSRVGRKPRNVLLAAMFTATFASMWISNVAAPVLCFGLIQPI
LRTLDPGHPFAKSLVMGIALASNVGGMTSPISSPQNIFAIERMSMDGQPPSWLSWFAVAIPVSITCNFLCWALLLLVYQPGRA
LGEVRPLKPNTDPINGTQVYIIVVSMLTVAAWCANTFLQRYTGEMGIIAIVPLVAFFGFDVLNKDDENSFLWNVVMLAMGGLS
LGEAVKSSGLLAALTNSISELVTGFTMFQVTLIFCGLVLLATTFISHTVGAMVILPIVQSVGESMAGTPHPKLLVMASALMCS
GAMGLPVSGFPNMNAVSLEDSTGNAIVSTKDFLYVGVPSSVMAYGIIVSLGYVLMLLVGM
>Ignatius_tetrasporus.PTC1
DMYSKLEDLVNFLELNREGFRKALKKHDKVTQRNLSPILLPEALEALNVQDNKNAIEERKQDVVQCYATSQQKGEVLAATNVL
KRQQREMVEFERSTVWREHMAVERQHAQATAKAVELTGWQHWWSSHRNLIWIAVAVAAFFIVLLVPMPIFDTVEQHNCAALLT
MAGILWCFEALPLFATGMLVPFLVVVLRVMRVADTPQHACTHDCRLSAPDAASAVFHSMMDQVIFLLLGGFTIAAALTKHNIA
KQLASAGLASVRDAPGKLLFAAMNIATVSSLFISNVAAPVLCFSLVQPILRTHKHDHPFSKALVIGIALASNIGGMTSPISSP
QNIFAIQKMDEDGRAPSWLVWFAVALPVAFACNAACFTVIWHFYKPYKTRTAIRLPKLTDKLNSTQVFVIVVSLLTVGLWCAN
AQLEKYFGKMGIIAILPVVLFYGSGVLNKDDLNNYLWNVVVLAMGGLALGEAVKSSGLLHVIANLLADAVGSLDLWLVLIVFC
GFVLIGTTFISHTVGAMIILPILQTVGKNLPGAPHPQLLVMGAALMCSGAMGLSVSGFPNMTAAAQQASTGEHYINSADELQV
GIPCSVITYGIIVTLGYGLMLAVGL
>Gonium_pectorale.PTC1
MKFTHQLKFNSVPEWREHYIQYAHLKKYIYALAKKEADLQAGAPTIEEGPLAPLLQDARATQGPSEEGFQRELDAQLAALLAF
FAVKEADLLAKVSGLELDVQSLEKIPGRREASTLSRLGITGGPCSSSDAIAPGAAVGATVAPLTTLTMDASPSTLDLARMVTC
TPPQDHRKVRVKYWENPPRSTFSPNLNSRKMKLQGRFQDLYIGLHDLRQFLIINKEGFRKIIKKHDKLTRMVDLRDCWWPNVE
AHLAPTTKQQELDRAIADISDHYAVVYTGGDVAKAEEQLSRGLREHITFERNTVWRDMAAMERKYAAVSVKQAAPPGGAKASR
LRDYLQWTKLALSCAVFAILLNVDVWPGPQNGPRNNCLALLVFASLLWSLEAVPLFVTSMAIPFLVVTLEVLTDGTKDPPQRL
TPQQAASVVFHAMFSQTVMLLLGGFSIAAALSKHAIAKQVAVTILSRVGRRPRNVLLAAMFTATFASMWISNVAAPVLCFGLV
QPILRTLNPGHPFAKALVMGIALASNVGGMTSPISSPQNIFAIERMSMDGHPPSWLSWFAVALPVSIIINLVCWALLLLVYQP
ERYITEVRRVKPNTDPVNGTQVYIVIVSLLTVVCWCGNSYLQRYTGEMGIIAIVPLVAFFGFGVLNKDDENSFLWNVVMLAMG
GLSLGEAVKSSGLLAALALDISNLVTGLSLWQVAAVFFGMVLVATTFISHTVGAMVILPIVQSVGEAMASPTHPKLLVMGAAL
MCSGAMGLPVSGFPNMNAVSLEDSTGNAIVSTKDFLLVGVPSSFFAYLVIVTLGYVLMLLVGL
>Planophila_terrestris.PTC1
MKFSHSLKFNSVAEWRQHYINYGALKKLSYAIEKQEEEGRQREMFAQQSMDLRQRSMRQSVDLPRGAVEEDEARRRASLDASS
SFTGSVQQPLLQRLSSGLGGSLRLSMLERSAEEGGKVSQADFQRSLDSELHKIVDFYITKEAELKKELAAAELDARAAEASSA
GSSGFAEMQAERLPRPSFWRTAASDALKAKMHERLCSLYVQLVDLLNFIELNRTGFRKILKKHDKVTDVSLMTDYMPVVTSKL
SSKREEDLGAMINEVIKLHAMVMHNSDTNASEVDLKRNLRDHVTYERDTIWRDMVALERRNITVKVPEGAPTGGFAKWWQIYH
TPVMVAAALLLFFVILNIDIWPNDTPKRRCAAMLALLVTLWTSEALPLYVTSMLVPLLTVLLRILPDEAAPDGHPQQLPAPKA
AEAVFKVMFSQVIMLLLGGFAIAGALSKHYIAKAMASNILSRVGTRPRDVILANMFVATFASMWISNVAAPVLCFSLIQPILR
TLPSHHPVSKCLVMGIALASNVGGMTSPISSPQNIFAIELMARDGAAPNWLTWFVVSIPVSIASNLFIWAILLAVYRPGLAIR
EVRHMRRVQEPITKVQVYVVAVSSLTVALWCASSALEQYLGSQGIIAIFPLVMFFGLGVLDKDDFNNFLWHVVMLAQGGLVLG
LAVKGSGLLADVAGAIRGVTAGMSLFGILFTFCALVLVCTTFISHTVGAMVILPIVRSVGQHLSPTPHPRLLVMGAALACSGA
MGLPVSGFPNMNAVSLEDATGKTYVNTLDFLMVGLLGSLTTFCVIVTLGYALMM
>Pteromonas_angulosa.PTC1
MKFTHQLKFNSVPEWRDNYIDYAHLKKFIFAIARAEQDDIQQLHGGADGTSMPLLQHTVTMGHDKVDATEDNLRQALDKELQR
VISFYMTKEAETLAKVTSMELEINTLEMTRAPRGTSMDHMQGAQRGGSGGSGGSGGGVDLNQQLPSPPQGLATDVEATPPAAH
VSASMAAPKSPGTMSRQMRVDFWARANPGARHGGSFGGGSAAFLFVRELQSHKERLRVVFSDLYLQLHDLLNFLRVNKEGFRK
IIKKHDKMTSSNLKEHYWPVLESKYPIVRADMLEATINSLVDLYAVIYNQGSVELAKDHLDKLLRDQIKVERNTVWRDMVAQE
RRTTAAVVEGAVRRPWWAQLTPHIALLSSVLVFAVLLSMEDIFEGEPEKQNCLALLIFVSMLWATEAVPLYVTSLAIPLLVVV
LKVLMDKSVDPPVRMTAQQAAPAIFHSMMSQVILLLLGGSAIASALTKHFIAKKLAQVVLSRAGRQPHNVLLALMLVAVVASM
FISNVAAPVLCWSLVDPILKAFDAENPFSKSLVMGIALASNIGGMTSPISSPQNIFAIERMSMDGHPPSWLAWFAVALPVSFI
CILVCWGLILAVYRPWTKVAEVRPLKPSSDKVTFTQFYVVAVTAVTVTLWCFNTQLQPYTGEMGVVATIPIIAFFGFGVLNKD
DFLSSPWLVMTLAMGGLGLGEAVKSSGLLLSIAHTIGDVVQGMDVFTVCCIFCALVLVCTSFISHTVGAMIILPIVQSVGEQM
PGPHHAKLLVMSSALMCSGAMGLPVSGFPNMFLISKDDGTGKNYINTLDFIKVGVPGSIGAYFVIVSVGYLLMLAV
>Asteromonas_gracilis.PTC1
MKFSHQLKENSVADWKEHYIHYANLKKIIYEIARLEQARANPDAGEVTELGEPLLSRPPVQNYELAISTKESEFVGELDRELA
HIITFILRKEAELVSQLEALDLEVHSLESADPQYRKSLDRDFLDQDAAVGAENGTGYQAGIPARPERIKFWSQGAEPHLAARD
ARNVAQLKPAQREALSQKFVDLFTTLNDLLEYLVLNREGFRKLIKKHDKMTSSASMKESYWPLIEQRYPEHKRVSMGQNIERL
VDLYAILFEGGDTSSAREALSQNLRQHIKVERNTVWRDMVAMERRTVAATVDAPKRKRAWFSTHRKHLSLLLASIVFASMLSL
KLFKEPEKSNCAAILVFVSLLWASEAIPLFVTAMVVPVLVVSLRVLVDDSSAKHPIRLSSTDAANAIFHAMFSQVTMLLLGGF
TIAAALSKHFIAKQMAVAVLSRVGRLPRNVLLASMCVAAFASMWISNVAAPVLCFSLVQPILRTLDVSSPFAKSLVMGIALAS
NIGGMTSPISSPQNIFAIERMSMDGVAPSWLSWFAVALPVSFISIILCWLLLLLVYRPGMSTTEVRPLKPYTDPMNMTQVYVI
VVSMVTVLLWCANSEVQQFVGNMGVVAVLPMIAFFGFGVLSKDDFNGFLWNVVMLAMGGSALGEAVKSSGLLSTFANDISGQV
HGLDLWTVSAIFCGVVLICATFISHTVAAMVILPIVQSVGEAMQENPHPKLLVMATALTCSAAMGLPVSGFPNMNAVSLEDGT
GQTFVNTLDFLKVGVPSSVATYFVIISAGYYFM
>Haematococcus_pluvialis-B.PTC1
RVRFWAELGMRRGGRDLRFARDVMRIRFHDLYTSINDLIEYLSLNREGFRKLIKKHDKLTSTCLKEAYWPDFERRYPMKRKEE
LERHLDRLIELYAVMFAGGDTRKARDLLLKTLREHIKVERNTVWRDMVALERRTVAATVGAASGVARLSKYKAYSERLGLLAA
LLVFAALLWAPVFEEKEKSNCLAILVLASMLWATEAIPLFATAMLIPVLVVMLRVLVDHGRPAGAQRLTPQEAAPLIFHAMES
QVIMLLLGGFTIAAALSKHFIAKQMAVAVLSRVGRKPHNVLLASMFVAIFASMWISNVAAPVLCFSLVQPILRTLDVNTPFAK
SLVMGIALASNIGGMTSPISSPQNIFAIERMSMDGNPPSWLSWFFVALPVAIISNFICWAAILLVYQPWHKTSEVRPIKPSSD
PVTWTQVYVIFVSLATVGLWCGNVALQKYTGEMGVVAVLPMVAFFGFGVLNKDDFNGFLWNVVMLAMGGSALGEAVKSSGLLL
TIAQGIQEMVDGLNLWTVTIIFCACVLVCTTFISHTVGAMVILPIVQSVGESMPGQPHPKLLVMSAALMCSGAMGLPVSGFPN
MNAVSLEDSTGQTYVGALDFIKVGVPSSILAYAVIITVGYSIMLIIGF
>Chlamydomonas_bilatus.PTC1
QTIMLLLGGFAIAGALSKHFIAKQLAIAVLSRVGRKPHNVLLAAMFVATFASMWISNVAAPVLCFSIIMPILKTLDTASSFAK
SMVMGIALASNVGGMTSPISSPQNIFAIERMSMDGQPPSWLAWFAVALPVATLCNLLCWLLILAVYQPWRTINDVRPLKPNTD
PMNFTQAYVIFISLATVGLWCANTSLQQYTGEMGVVAVLPLVAFFGSGVLSKDDFNGFLWNVVMLAMGGLALGEAVKSSGLLQ
SMAEGITEVTDGMDLYQVLLVFCLMVLISTTFISHTVGAMVILPIVQSVGEAMPGSPHPKLLVMASALMCSGAMGLPVSGFPN
MNAVSLEDSTGQNYVDTLDFLKVGVPGSVLAYGVIVTLGYNLMLMVRF
>Vitreochlamys_sp.PTC1
MKFSSLLKFNCVPEWRDHYVQYGHLKKYIYALAKWEADHLHETQPPDLESLTSPLLPTSGLGSAYGPSEEAFQRELDQSLLEV
IRFFSMKEAELVSKCQALLLELVSVEKLPSGSSAGRRSFSGASTPSGAATPTSSAPHGSTANVLAGAKSRLTASPQTSPHVTL
SGAKGAGGLGGMHLSPSVVHLMDVANHKDHRTVRVEFWRKPPRRLFQNLEAARSKLKPRLQELYIALHDLAEFLHLNREGLRK
VVKKHDKLTRRVTLKTKWWPQVEHLIPPTKKEEVDRAVSELVDNYAVLFTGGSMAAAEQALSQGLRDYVTMERNTVWRDMAAM
ERRFASLAVKKGSASFIATWWTQPLKIAVSLLVLSVLLNVTIWPEDEKNNCLAVLLFASMMWSLEAIPLFVTSMTIPFLVVCF
QLLVDHSQDPPVRMTAQQAAPAIFHAMFSQVIMLLLGGFAIAAALSKHAIAKQISVAVLSRVGRKPRNVLLASMFVATFASMW
ISNVAAPVLCFGLIQPILRTLDPGHPFAKCLVMGIALASNVGGMTSPISSPQNIFAIERMSVDGKAPSWLSWFAVALPVSIVS
NLICWAVLLLVYRPWTKIQEVRPIKPITDPINGTQVYIIVVSVVTVALWCSNTILQPYTGEMG
>Botryococcus_terribilis.PTC1
MKFSQQIIFNSVPEWKDNYISYAQLKRLIYSEEAARLAAGRDGARGASMRLQRLRKTAMQFKDDLKKEADKVVRFFHEEVESI
WSRFHLVLHEIECFEQQEWLPPSAAGLDTSPTSPLLSATSMPATPKTPPMPSPRSSPFQRAGSAGMGLLRTVTGILPRPKRSP
RTLSGPLLEVEDGTPRDDSKTWIWQQAEPSIARKRDELRGQLSEVYQDANNMIEFRRLNLDGFRKILKKYDKVLEGLPGAEKL
SESQFPGIKERLEALDLTRMQEVEGEVVRLYARVCCSGVYAVAEELLKKQKKDRIVEDRSTVWKEMVERERKRSAAHVEGGAA
PRAWYQRHWQLMACAFCGAVFLALLWVPIFEEVEKQHCAALLAFVSLLWCTEALPLFATAMLVPFLVVTLGVLVDRSVDPPHR
LTPQEAAPAVFKTMMSQVIMLLLASFAIAGALSKHFIAKWLASVFLSRFGKRPSRVLLANMGVATFASMWISNVAAPVLCFSL
LQPILRNLSAKDSFAKALVLGIALASNLGGMTSPIASPQNLFAIQQMSVGGNAPSWLQWWLVALPVAIIGNLVCWGLLLWRYQ
PPPDDVRELHEAKGFHINPTQMYVVAVSLLTVGLWCCNGYLTPYFGEMGVIAIIPLVAFFGTGVLDKDDFNAFLWNVVILAMG
GMALGSAVDSSGLLLTIAKKLEGLVSSHGPWVVLAIFCALVLFATTFVSHTVGAIVILPIVRAVGETMTDPHPKMLVMGAALM
CSGAMGLPVSGFPNMNAISLEDKTGVNYLTTKDFLLVGVPSSVATWGIIVSVGYVLM
>Eudorina_elegans.PTC1
MKFTHTLKFNAADSWREHYIQYAHLKKYIYALAKREADLQAGGHVPDDESLHAPLVPETSRSGQGVSEEGFQRELDAQLAAIL
SFFAVKEAELLAKVSELELDLQSLEKIPNRQEASTMSRLGGGGGAAGSNPTGSPGTAAVAAVSAVLPSLSILSVSPSTLDLAR
MVNSTPPEEHRKVRVKFWENPPRHVFLPSLHARRTKLQGRFQDLYIGLHDLREYLHINKEGFRKIIKKHDKLTRAVDLRVRWW
PNVEAHLAPDAKQQELDRAIAALTDHYAVLYMGGDVAKADEQLSHGLREHITVERNTVWRDMAAMERKYAAVSVKQAAAPGGL
RGSYRKLAACCAVFAVMLHVKVWGEDEDEPKNNCLALLAFASLLWSLEAVPLFVTSMALPLLIVVTGVLVGPDKQPLTPQQAA
PAIFHAMFSQTIMLLLGGFAIAAALSKHAIAKQVAVAILSRVGRKPRNVLLAAMFTATFASMWISNVAAPVLCFGLIQPILRT
LDPGHPFAKSLVMGIALASNVGGMTSPISSPQNIFAIECMSFDGHPPSWLSWFAVALPVSITCNFACWAVLLLVYQPGRAIAE
VRPIKPNTDPINGTQVYIIVVSLLTVAAWCANTFLQRYTGEMGVIAILPLVAFFGFDVLNKDDFNSFLWNVVMLAMGGLSLGE
AVKSSGLLAALASDISGVVKDLTLFQVAVIFCGMVLVATTFISHTVGAMVILPIVQTVGKAMEGTPHPKLLVMAAALMCSGAM
GLPVSGFPNMNAVSLEDSTGNAIVSTQDFLYVGVPSSIIAYGIIVTLGYVLMLLVGL
>Pandorina_morum.PTC1
MKFTHQLKFNSVPEWREHYIQYAHLKKYIYALAKREADLQAGGDEDGLLSPLVPETSRAGQGVSEEGFQRELDAQLASILSFF
AVKEAELLAKVSELELDVQSLEKIPSRQEASVSLSRLGAGGGSGGGNPTSSPGSAAVSAVSAVLPSLSLLSVSPSTLDLARMV
SSTPPEEHRKVRVKFWENPPRHVFSPNLHARRAKLQGRFQDLYIGLHDLREYLHINKEGFRKIIKKHDKLTRAVDLRARWWPN
VEAHLAPDAKQQELDRAIAALTDHYAVLYTGGDVEKAEEQLSRGLREHITVERNTVWRDMAAMERKYAAVSVKQAAAPGLLRF
SANRAHVRWAKLALCCVVFAILLNVDFYKENDMEPPDVQRAKNKCLALLVFASMLWSLEAVPLFVTSMALPFLIVMLGVLMDS
DGKERLQPKSAAPAIFHAMFSQTVMLLLGGFAIAAALSKHAIAKQVAAAILSRVGRKPRNVLLAAMFTATFASMWISNVAAPV
LCFGLIQPILRTLDPGHPFAKSLVMGIALASNVGGMTSPISSPQNIFAIEEMSKGANPPSWLSWFAVALPVSIACNLICWAVL
LLAYRPGHVISEVRPIKPNTDPINGTQVYIIVVSLLTVAAWCSNTFLQRYIGEMGVIAIVPLVAFFGFDVLNKDDENSFLWNV
VMLAMGGMSLGEAVKSSGLLSALATDISNLVLHLSIFQITVIFCGMVLVATTFISHTVGAMVILPIVWSVGEKIKGDDPASQS
HSKLLVMAAALMCSGAMGLPVSGFPNMNAVSLEDSTGNPIVNTQDFIYVGVPSSIFAYGVIVTLGYVLMSLVGF
>Oedogonium_foveolatum.PTC1
VKFTHYLKENSVPEWRGQYLAYGLLKKLIYKQEKLLALSRAAPHPESVDIEHEEPTVETPFLQVPSTPPSQLDLSPRRSFDRS
FLSGKLSPRSASTTGNPEIEFVRLLGSERTRLNEFIASKYTELTGQLSNVTEVMRVKEMEGGLPHSDPNPYSLAAHRVAFWSQ
APMQKAREQLIPQLVELCVFLTGLKDYVEMNKEGFRKILKKWDKVNEARLSEQEMPLVEQTLDVGRRLQDLDEAIGHVMSLYA
LLTSKGNMDLAWRSMKEHQSEHIRFQRSTVWHDLIALERRTLTATAVRPVDEVMGWWAVNRKHEMIVASLMVFLLLLEAKTFE
GDEAAPQRNCLALLVFVSCLWATEAIPLFVTSMLVPLLAVSLRVVVVDGKRLEPPDAATFMFGKMFSQVIMLLLGGFAIAAAL
SKHNIARKMAISVLSRVGRAPGRVLLATMMVATFLSMWISNVAAPVLCFSLVQPILRTLDTNHQFAKALVMGIALASNVGGMT
SPISSPQNIFAIQVMSGGGHSPPSWTQWFVVALPVSAVCNVLIWGLLLAVYQPHKHIKEVRPIRALQDAFTLQQVVVVLVSLL
TVTLWCLNGMLEPYLGSMGVIAILPLVAFFGFGILTKDDFNAFLWNVVMLAMGGLAVGECVKSSHLLQSIARGIQDTTAGWSL
YCVLAMFCALVLCCTTFISHTVGAFVILPVLQSVGDEMAAAGQPNHSKLLVMAAALMCSGAMGLPVSGFPNMNAVALEDQAGF
NYVATIDFIKVGLLSSGFAYVVIISLGYLLMLMVGF
>Chlamydomonas_sp.-M2762.PTC1
PARRLDKLDAAIAKLVDLHAVIYLAGDATKAKDQLSRVLRDVERNTVWRDMVAMERRAVSATVEGTKRPPWWKGYTEHMGLVL
SVAVFAVLLSVEIFDEEEKNNCLALLAFVSMLWATEAIPLFATSMLVPPLVVILRVLVDRTKDPPVRLTAQQAAPTIFHAMES
QTIMLLLGGFAIAAALSKHFIA
>Chlamydomonas_noctigama.PTC1
MKFTHQLKFNTVPEWRDHYIHYAALKKIIYAIAKAEADEHQHPAGHDDEHLGVALLDKVEATEEYLIKSLDKELAEVIKFYMA
KEAEILGKLEQLDLEVHSLEQRSALGTTLRSTSMPLPSDAVPVILEEDDLSRTESVRASRTEFWRTNSRSLKPTSRALIKDSG
KMKQRIIDLYSSLHDLADFLNFNKEGFRKILKKHDKVTSSNLKDRYWRVVEDKYPSKKAEVLEQAMDRLTDQFAVLYLQGDTV
KAKDTLGRVLREQIKVERNTVWKDMVAMERRTVAAVIKPGAAEPKKVSFFAKHHSRIMLLLSVVVFASLLSVEIFPEPEKQNC
LAMLVFVSLLWATEAIPLYATSMLVPPLVVLLKVLVDRSHEEPIRMTAQQAAPTIFHAMFSQTIMLLLGGFAIAAALSKHFIA
KQLAIAVMSRVGRKPHNVLLASMFVATFASMWISNVAAPVLTFSIVMPILKTLETSSAFAKSMVMGIALASNIGGMTSPISSP
QNIFAIERMSMDGQPPSWLSWFAVSLPVSSVCIILCWLLILAVYQPWRSVSDVRPLKPNTDPMNMTQVFVIVISMATVGLWCA
NTALQSYTGEMGVVAMLPLVAFFGFGVLSKDDFNGFLWNVVMLAMGGLALGEAVKSSGLLQSIAEAIKEVTDGYDLYQVLLVF
CVMILVCTTFISHTVGAMVILPIVQKVGEDMPGPHPKLLVMAAALMCSGAMGLPVSGFPNMQAVSLEDSTGQNYVDTLDFLKV
GVPGSVLAYLVIVSLGYTLMLLVRF
>Carteria_crucifera.PTC1
QVYVIVVSVVTVVLWCLNSALQNVTGEMGVIAIIPMVAFFGTGVLSKDDFNGFLWNVVMLAMGGLAMGEAVKSSGLLAAIAEG
IKELVAGMDLWEVLAIFCSLILVCTTFISHTVGAMVILPIVQSVGEMALGHPHPRLLVMGSALMCSGAMGLPVSGFPNMNAVA
LEDSTGVNYVSTVDFLWVGIPSSIFAYVVIVTVGYFLMLMVRF
>Volvox_aureus-M2242.PTC1
CLALLVFASLLWSLEAVPLYVTSMALPFLIVAMGVLVDHPNDSKDPPKRLTPQQAAPAIFHAMFSQTIMLLLGGFSISAALSK
HAIAKQVAVAILSRVGRKPRNVLLAAMFTATFASMWISNVAAPVLCFGLIQPILRTLDPGHPFAKSLVMGIALASNVGGMTSP
ISSPQNIFAIERMSMDGQPPSWLSWFAVAIPVSITCNFLCWALLLLVYQPGRALGEVRPLKPNTDPINGTQVYIIVVSMLTVA
AWCANTFLQRYTGEMGIIAIVPLVAFFGFDVLNKDDENSFLWNVVMLAMGGLSLGEAVKSSGLLAALTNSISELVTGFTMFQV
TLIFCGLVLLATTFISHTVGAMVILPIVQSVGESMAGTPHPKLLVMASALMCSGAMGLPVSGFPNMNAVSLEDSTGNAIVSTK
DFLYVGVPSSVMAYGIIVSLGYVLMLLVGM
>Phacotus_lenticularis.PTC1
MKFTHQLKFNSVPEWRDQYVDYAHLKRFIYAIARAEQDDIQQLHEVHDTTMPLLPHTVTMGHDKVEATEENLRQALDKELQRV
ISFYMAKEADILAKVTALELGIHALEKLPARGVSLELDPTRQGSQVAAGGVAGGGAPPGGRHVPLLQGAPSVTREGSGGIAHS
ISPQQSSSSPQLGGPGSGRTAGTGGSQSASPQPSGAMHGGDLEAPLQGGDHTGKTSPHWSRAARVEFWGRAQPAHRFTGGASF
SAASFSGPFVRDMQAHKERLRPQFSDLYLSLHDLLGFLRLNKEGFRKIIKKHDKMTSSNLREQYWPLLEAKYPIQRAELLEAT
IASLVDHYAVIYLGGDVGTSKAHLDKVLRDQIQVERNTVWRDMVAQERRTTAAVVATTYKQKVWAKVTPHIALVSSVAVFALL
LSVEDLFPEAPEKQNCLALLIFVSMLWATEAVPLYVTSLAIPLLAVTLRVLVDKTTDPPQRMPAQQAAPAIFHSMCSQVILLL
LGGSAIASALTKHFIAKKLAQVVLAQAGRQPHNVLLALMLVATVASMFISNVAAPVLCWSLVEPILKSFDADNPFSKSLVMGI
ALASNIGGMTSPISSPQNIFAIERMGMDGHPPSWLSWFAVALPVSFICILVCWGLILGVYRPWSKVAELRPLKASADKVTFTQ
IYVVLVTVVTVGLWCCNTMLQPYTGEMGIVATIPIIAFFGFGVLNKDDFLSSPWLVMTLAMGGLALGEAVKSSGLLLSIAHSI
GDLVQDLDLFTVCVIFCGLVLVCASFISHTVGAMIILPIVQSVGEQMPGPHHSKLLVMASALMCSGAMGLPVSGFPNMFLISK
DDGTGKNYVNTLDFIKVGVPGSVGAFFVIATVGYVLMLMV
>Stephanosphaera_pluvialis.PTC1
RRQRVRFWASLDTRAELRDLRLVRGMMRYRFNDIYTTLNDLMEYIMLNREGLRKVVKKHDKLTTTVALKESYWPTVDQQLALS
KRDAMAQQIEQLVDLYAVMFTAGDVDAAKELLSKNLREHIKVERNTVWRDMVALERRTVAATVQQTTGKAAAKLQRYREPLCL
LLSLAAFFALLRAAPFAEPEKNQCLALLALCSLLWATEAVPLFATALAIPPLVVVMRVLVDRSDPAAPHRLTPQQAAPAIFHA
MFSQVIMLLLGGFAIAAALSKHFIAKQMAVAVLSRVGRKPHNVLLAAMFVATFASMWISNVAAPVLCFSLVQPILRTMDVTTP
FAKSLVMGIALASNIGGMTSPISSPQNIFAIERMGMDGHPPSWLAWFAVALPVAIISNLLAWGLLLLVYRPWTHTTEVRPLKP
SSDPINLTQVYVCLVSLATVGLWCANTALQKYTGEMGVVAVLPLVAFFGFGVLNKDDFNGFLWNVVMLAMGGSALGEAVKSSG
LLVSIAESIRQLVAGMDLWMVTVVFCLAVLFCTTFISHTVGAMVILPIVQSVGEAMPGPPHSKLLVMASALMCSGAMGLPVSG
FPNMNAVSLEDATGQTYVSASDFIAVGVPSSVAAYAVIVTVGYSLMLLVGF
>Chlamydomonas_eustigma.PTC1
MKFTHQIKFNSVPEWRDHYIDYAHLKKIIYAIAKAEADEQQQHHLDEEHPLLTRQQTAHGEKVEATEEALIQALDKELAKIIK
FIMAKEAETLGKLAQLDLEVHSLEAQRVGSMFTPPIVNRFTSLQDAGNTRLGGSLPDPQKDGFETLGLADRRPSEVMEEAVRP
DLEGGIGSNSFRASRVHFWHSNSLPATTRTGARVLAKDSAKMKPRITDLFVVLHDLKNYLSLNKEGFRKILKKHDKMTSSNLK
SRYWCIIEEQYPSKKEEGIMQAINKLVDLYAVLFLKGDFEKASSVLNRVLGEQIKVERNTVWRDMVAMERKTVNAAVHKPQGV
ATRVTWLQQNMKHILLMLAVLTFATLLTVQTFEEPEKNNCLAMLVFVSMLWATEAIPLFATSMLVPPLVVILRVMVDHTKSPP
ERMPAKDAAPAIFHSMFSQAIMLLLGGFAIAAALSKHYIAKQLAISVMSRVGRKPQFVILAAMCVAAFVSMFISNVAAPVLTY
SIVMPILKTLDTGCPFGKALVMGIALASNVGGMTSPISSPQNIFAIQLMSNDSNPPSWLAWFAISLPVSALCVLMCWSLILIV
YQPWRRVAEVRPLKPSTDPINGTQVYVIIISLATVALWCANTVLTPYTGEMGVVAVLPLVAFFGFGVLSKEDENGFLWNVIML
AMGGMAVGEAVKSSGLLHSIALGIQDLTSGLDLFQVMIIFCLLVLICTTFISHTVGAMVILPIVQSVGESMPGTAHPKLLVMA
TVLMCSGAMGLPISGFPNMQAVSLDDGMGQNYVSTIDFLMVGVPSSVLAYFVIVSVGYSLMLLVRF
>Chlamydomonas_incerta.PTC1
MKFTHQLKFNSVPEWREHYIQYGHLKKYIYALAKREADLQAGGQEEEALLAPLLLEAGRDQGPTEEGFQQELDAQLAATLSFF
AVKEADLLAKVSALELDIQSLEKIPNRAEASTLARMGMGMGGSASPGGPMSSPRAAAAAAMSAVASLVSHSPSTLDLARMVNS
TPPEDHRKVRVKFWENPPRHLFSTNLSARRAKLQARFQDLYISLHDLREFLHINKEGFRKIIKKHDKLTRAVDLRARWWPNVE
AHLAPAAKQAELDGAIAALTDHYAVLYTRGDVAQAEEQLSRGLREHITVERNTVWRDMAAMERKYAAVSVKQAAAPGARVTWL
RTHARWLKLALSVAVLVVLANVEVWPGPENEPRNNCLALLVFASLLWSLEAVPLFVTSMALPLLIVAMGVLVDRSKDPPQRMS
PQQAAPAIFHAMFSQTIMLLLGGFSIAAALSKHAIAKQVAVAILSRVGRKPRHVLLAAMFTATFASMWISNVAAPVLCFGLIQ
PILRTLDPGHPFAKALVMGIALASNVGGMTSPISSPQNIFAIERMSLDGSPPSWLAWFAVALPVAVAANFVCWGLLLLCYQPD
KAIAEVRPIKPNTDPINGTQVYIIVVSLLTVAAWCANTFLQRYTGEMGVIAVVPLVAFFGFDVLNKDDENSFLWNVVMLAMGG
LSLGEAVKSSGLLAALALTISDLVTGLSLWQVATIFCGMVLVATTFISHTVGAMVILPIVQSVGEAMPGTPHPKLLVMAAALM
CSGAMGLPVSGFPNMNAVSLEDSTGNAIVGTGDFLAVGVPSSVFAYGIIVSLGYLLMLAVGF
>Chlamydomonas_schloesseri.PTC1
MKFTHQLKFNSVPEWREHYIQYGHLKKYIYALAKKEADLQAGGHDDEEALLAPLLEAGRDQGPTEEGFQRELDAQLAATLSFF
AVKEADLLAKVSALELDIQSLEKIPNRAEASTLARMGGPGSAMASPGGGGPMASPRAAAAAAMSAVASLVSHSPSTLDLARLV
NNTPPEDHRKIRVKFWENPPRHLFSTNLSTRRAKLQARFQDLYISLHDLREFLHINKEGFRKIIKKHDKLTRAVDLRARWWPN
VEAHLAPAAKQAELDGAIAQLTDHYAVLYTRGDVAQAEEQLSRGLREHITVERNTVWRDMAAMERKYAAVSVKQAAAPGARVT
WLRTHARWLKLAGAVLVFLVLANVQVWPGAENEPRNNCLALLVFASLLWSLEAVPLFVTSMALPLLIVALGVLVDHTKDPPQR
MTPQQAAPAIFHAMFSQTIMLLLGGFSIAAALSKHAIAKQVAVAILSRVGRKPRNVLLAAMFTATFASMWISNVAAPVLCFGL
IQPILRTLDPGHPFAKALVMGIALASNVGGMTSPISSPQNIFAIERMSLDGRPPSWLAWFAVALPVAVACNFVCWGLLLLCYQ
PGKAIAEVRPIKPNTDPINGTQVYIIVVSLLTVAAWCANTFLQREVHKSIYATTGEMGVIAVVPLVAFFGFDVLNKDDENSFL
WNVVMLAMGGLSLGEAVKSSGLLAALALSISDLVTGLSLWQVATIFCGMSAAKLWSPPGGRSNQPGPRQQPQKGYCWYNNAGP
SGLTNH
>Chromochloris_zofingiensis.PTC1
MKFSQTLKFNRRPDWEIHYINYAHLKRLITKVQQAEFAEQNNLPLHFGDEEAGVRSPLLSQTSFNRQQSVSAALTRQQSFTIS
AAQCDEAFIKALDSELARIIQFYMRKESELLARFESAALRIHSIEGPALPGPAALDTAQRIQFWSQDTKEIALEREKLRSEMT
DLYEQLHALSKYLELNFTGFRKILKKHDKMTSQNQYKDSYMPIVEAKLPLKNREMISGVINNLVEMYAVVCTRGDVNRAQAEL
KRKLKDEVAFERSTVWRDMVAMERRGASVAVHEASSLADQPKKPRWWQAHRQLLLVTLCVTVFAVLLSVPIFQQPEKQNCLAL
LAFVSLLWCTEAIPLFVTSILVPLLIVVLRVLVDRSADPPRRLPPQEAAPAVFHVMFSQVIMLLLGGFAIAAALSKHFIAKQL
AVAILSRVGRKPQYVLLANMLVATFASMWISNVAAPVLCFSLVQPILRTLSPSHAFAKSLVIGIALASNLGGMTSPISSPQNI
FAIERMSMDGNPPSWLSWFAVALPVSVLGNLLCWGLILLVYNPGATIKEVRPVKPPEDPLNGTQIYVILVSVATVGLWCFNSF
IQHVTGEMGVLAILPLVAFFGFGVLDKDDFNGFLWNVVMLAMGGLALGEAVKSSGLLLTIATGIQDFVAGLGLWSVLAVFCFL
VLICTTFISHTVGAMIILPIVQSVGETMSGTPHPKLLVMGSALMCSGAMGLPVSGFPNMNAVALEDPTGQNYVNTIDFLKVGV
PGSIMAYGVIVSLGYVLMIAVGM
>Coccomyxa_subellipsoidea.PTC1
MKFGAERAGHALLSWLTAAWLWLLQAWEVVAEWGRQCWGALLHAWHYIASAVMQAVHWQTENRIADLGRIPEEVGGDLDRTIS
LALEEGGDDIKGAFDSELNRITTFHKKKEEELLGAVDKLGEEVSSAVEPSAQQSAPDASSPLLGTSRNAEALYWGQDTVAVRI
AREQLRETFQELYVEIQGLIDFVEVNRTGFRKALKKHDKVLGALGHPKMQPTYMPNVEAAFPEKNRLRVSEAQKQLVELYAVV
CCHNNLLLAQLELKAQLRSQLKLERTTVWKDMVEKERKENAATVDDSGAESKPWYRSSLFMIALSCVVFAVLLSVPIFEERAK
QNCLALLGFASMLWCTEALPLYVTSMLVPLLAVVLRVMVDDSGKHPVRKSAPDAADAIFKAMFSQASSQLFISPHCTIERHVD
GLPSYPTTIMLLLGGFAIASAFTKHFIAKRVAVWVLGKVSAKPHAVLIANMFVATFASMWITNVAAPVLCFSVLDPILRTLPS
GHSFGKALVLGIALASNLGGMTSPISSPQNIFAIQEMGRDGEPPSWLAWFAVALPVACVGNFACWGFLLLAYRPGRTLKEVRR
MPFSSDPFTWKQIYVVVISLGTVGLWCANTALSKFTGQMGIVAIVPMVAFFGFGLLSKDDENNQLWNVVMLAMGGSALGEAVK
SSGLLSSIAHSIEDVVAGMGVWAVFAIFCALVLVATTFISHTVGAMVILPIVSAVGAQMEEPHPRLLVMGAALMCSGAMGLPV
SGFPNMTAYAKEDPTGNPWLSTIDFFKVGVPCSLATYGLIVTVGYGIMKFVLGW
>Coccomyxa_subellipsoidea.PTC2/homologue
MKFGAERAGHALLSWLTAAWLWLLQAWEVVAEWGRQCWGALLHAWHYIASAVMQAVHWVRGLEEVGGDLDRTISLALEEGGDD
IKGAFDSELNRITTFHKKKEEELLGAVDKLGEEVSSAVEPSAQQSAPDASSPLLGTSRNAEALYWGQDTVAVRIAREQLRETF
QELYVEIQGLIDFVEVNRTGFRKALKKHDKVLGALGHPKMQPTYMPNVEAAFPEKNRLRVSEAQKQLVELYAVVCCHNNLLLA
QLELKAQLRSQLKLERTTVWKDMVEKERKENAATVDDSGAESKPWYRSSLFMIALSCVVFAVLLSVPIFEERAKQNCLALLGF
ASMLWCTEALPLYVTSMLVPLLAVVLRVMVDDSGKHPVRKSAPDAADAIFKAMFSQASSQLFISPHCTIERHVDGLPSYPTTI
MLLLGGFAIASAFTKHFIAKRVAVWVLGKVSAKPHAVLIANMFVATFASMWITNVAAPVLCFSVLDPILRTLPSGHSFGKALV
LGIALASNLGGMTSPISSPQNIFAIQEMGRDGEPPSWLAWFAVALPVACVGNFACWGFLLLAYRPGRTLKEVRRMPFSSDPFT
WKQIYVVVISLGTVGLWCANTALSKFTGQMGIVAIVPMVAFFGFGLLSKDDFNNQLWNVVMLAMGGSALGEAVKSSGLLSSIA
HSIEDVVAGMGVWAVFAIFCALVLVATTFISHTVGAMVILPIVSAVGAQMEEPHPRLLVMGAALMCSGAMGLPVSGFPNMTAY
AKEDPTGNPWLSTIDFFKVGVPCSLATYGLIVTVGYGIMKFVLGW
>Symbiochloris_reticulata.PTC1
MQLGLGRDDMQRLFVLLTGLERYIDLNIAGFRKALKKHDKVLADAESGKLKETYMPTVHRQCCLNKKPILETLYAIVCCDGNN
EMALIDLKRRLGETVQFERNTVWKDMVQKDRKRGTLKVDDGLIGSWWHRARQPAAIAMSLAVFVVLLYTPTFREPEKRNCLAL
LAFTSLLWCTEALPLYVTSMLVPLLVVVLRVLVDGSQHPPQRLSCKQAAPHIFHAMNSQVIMLLLGGFTIAAALSKHAIAKIL
ASWVLSKVGQRPGAVLMANMLVATFASMWISNVAAPVLCFSLVQPVLRTLDATHSFAKSLVMGIALASNLGGMTSPISSPQNL
FAIERMSMAGLPPSWLSWFAVALPVAFLGNFLVCGLLLLVYQDPHFTEVRPMQPIKDPINGKQMYIIAVSVGSVTMWCENSVL
QQWFGEMGIIAILPMIAFYGFGILDKDDFNSMLWNVVMLAMGGLALGEAVTSSGLLLSIAEQLQHLVQGASVWRVLVIFCGLV
LVATTFVSHTVGAMVVLPIIQSVGSQLSDPHPKLLVMGAALMCSGAMGLPVSGFPNMNAVALEDSKGINYLTTIDFFKVGLLS
SLIAYGLIVTLGYGIMYYGIGW
>Edaphochlamys_debaryana.PTC1
MKFTHQLKFNSVPEWREHYIQYAHLKKYIYALAKKEADHQADGAGTGDVEGLIAPLLQDGGRASGPTEEGFQRELDSQLAALL
GFFAVKEADLLAKVSELELEVQSMEKIPNRNEASNLVRARGGGSAASGTPSPGASPRASAAGAALSALSGLLAASPSTMDLAR
MVAASPPEDHRSVRVAFWKNPPRHLFSSSLQSRAAKLQSRFQDLYIALHDLREFLHINKEGFRKIIKKHDKLTRSVDLRARWW
PNVEAHLAPAAKQAELDGAIAGLTDTYAVVYCRGDASSAEELLSRGLREHITVERNTVWRDMAALERKYAAVSVKQAAGAAKP
SWLWRHARWLKLGFALAVFGIMLQYEVWPGPENAPRNGCLALLVFASLLWSLEAVPLFVTSMLLPLLIVLLGVLVDRTKDPPQ
RMTPQQAAPAIFHAMFSQTIMLLLGGFAIAAALSKHAIAKQFAVAILSRVGRRPRNVLLASMFTATFASMWISNVAAPVLCFG
LIQPILRTLDPGHPFAKALVMGIALASNVGGMTSPISSPQNIFAIERMSLDGRPPSWLAWFAVALPVSIACNFVCWGLLLAVY
RPERVIAEVRPIKPNTDPINGTQVYICAVSLLTVGAWCANTFLQKFTGEMGVVAVVPLVAFFGFDVLNKDDENSFLWNVVMLA
MGGLCLGEAVKSSGLLAALALGISDLVTGLSLWQVAVVFCGMVLVATTFISHTVGAMVILPIVQSVGEAMPGTPHPKLLVMAA
ALMCSGAMGLPVSGFPNMNAVSLEDATGNAIVATQDFLLSGVPGSIAAYGIIVTLGHHTMALLAAP
>Enallax_costatus.PTC1
MKFTHVLKFNSVPEWRESYINYPLLKKLILAASTAEYHEAYEGLALTQDEEAGPRSPLLSAQPSLSRSLSVTMTREQREKEFL
EALDNELAKIIRFYLKKEAEISAKFEELSMMVHHAEGIPSPTPEQMADGHDVTTAARVAFWSQGGRAVAAQREKLKTSLEELY
ATTFSLANYVEQNRTGFRKILKKHDKLVSHTMSSNYLPIVDQKFPASHAATLHHQLEAITALYAVVCCNGNLEHANSILRKQQ
QEQVSFQRNSIWKDMVGQERRAATVRVQDGKEVEPESWFTAHRQAVILAIALAVFVVLLTVPIFKQPEKQNCLALLAFASMLW
CTEAIPLFVTSMLVPFLVVVLQVLDDVTQEPPERLTPKQAAPRVFHTMFSQTIMLLLGGFAIAAALSKHFIAKQLAVAILSRV
GRKPHHVLLANMLVATFASMWISNVAAPVLCFSLVQPILRTLPTTHAFCKSLVIGIALASNLGGMTSPIASPQNIFAVERMGM
GGTPPSWLEWFAIALPVSFLGNLLCWGLLLLVYKPGKDIKEVRPLKPTEDPLTGTQIYVIVISLATVTLWCCNSFLQEYTGEM
GVLAIFPLVAFFGFGVLNKDDFNGFLWNVVMLAMGGLALGEAVQSSGLLLEISNSISHLVAGQSLWAVLAIFCGLVLVGTTFI
SHTVGAMVILPIVQAVGQQMPGGDHSKLLVMGAALMCSGAMGLPVSGFPNMNAVALEDPTGANYVYTKDFLLVGVPGSIMAYG
IIISVGYLLMLAVGF
>Mesostigma_viride.PTC1
MKFGKVLKDDAVPDWIPKYVAYKKLKRVVQRMELTVEQELQQAASKRGAAGSSDVTSPLATKETLLQRKSDEFMEGVEEEVAK
VNHFYDEMVSALRCDLEAYEKQLAAQLAGGNKKAFQKMFVLASDLNAYITLNSTAFRKIMKKHDKLTGLHRMDAFVARIKHEG
FMEAKALRELSARLEAMMSPDALDSLKQQYHLERQKRSESAGGSTGSPAKPTRILFSIAVFFLILALPPFWSARPASGGNDDG
IADVSDGAGVSGGVAFGVDYGYEGEPASLGAQGGVGEAAVAARDRLMRVLWERHYARDEAASSSIGDYVSGNSAFGPTQEERA
HRCFALLIFIACMWVLEALPYFVTSLMIPPLVVMLNIMADPTDKDKALSAPDSSRLVLSSMFDHVLILLLGGFTLSAAFGQCA
FELRIAGALQRALGHRPWLFMLAIMLLSLFLCMWLSNVTAPVLMLSVLLPILRDFDHGGRYPKALLLGLAFACNLGGMVTPIA
SPQNAVALVALDAQHFTITFFEWMAVALPFCVLLVVVVWAYLIFALRPDDVVSIPPVMYKTTPLSSKHIWVLLFSLATIGLWS
TLSLTVSVLGDLGIIALLFMVFAFGTGVLSKHDLNSFSWHLLLLIAGGNVLGRAVQSSGLIQIVAQIVTPYLHDILWVAALEL
LAFMIIITTFVSHSVAAIIMMPLIVAIGKEISPLSAEVLVLLCTLADSAAMALPMTSFPNVNSLLVEDDYGVPYLRVVDFIKV
GAPVSIMVVTAIATLGYSLAVFVLRP
>Raphidocelis_subcapitata.PTC1
MKFTHQLKFNAVPEWKEHYINYPLLKKIIYATRAAECQDAYDGVGGDEEAAGPSASGGSLLRSPRTSLSGGSLRAPLLQGVGG
LSLSRSGSVGARAGDSEFIKALDQELARIISFYLRKEGELTSAFESLNLQLHSRDGCDAAAPAAGGAGGGGGGAAGFGTAPAA
PAAGAVDGAAAAEAGEAAAAAAVPQSQAERQRRAEFQRRTAYWAANDRGVAAERERFRQKLVGLFVQLDGLKKYLEMNHTGER
KILKKHDKETTQHQYKDSYMAIVDAKLPLRSLEGLNRLIERLREMHAAVCCKGNLEKAERELRSELREEVGFERNTVWRDMVA
MERRTGAVVLQEPAHGIADESRQEPWLRRHWQPLALCVSGLAFAALLAAPLFEGAPEKRNCLAMLAFVSLLWCTEALPLFVTS
MLVPLLVVVLRVLVDRTVEPPVRLEPQQAAPAIFRVMFGQVIMLLLGGFAIAAALSKHFIAKQLAVAILSRVGRRPRDVLLAN
MLVATFASMWISNVAAPVLCFSLVQPILRTLPPSHPFAKSLVIGIALASNLGGMTSPISSPQNIFAIERMSMDGHPPSWLAWF
AVALPVAFAGNVLCWGLILAVYRPGQKIREVRPLKPPEDPLSPTQVYVVVVSLATVALWCCNSLVAGVTGEMGVLAILPLVAF
FGFGVLSKDDFNGFLWNVVMLAMGGLALGEAVKSSGLLLTIAQSVGQQLPGPPHDKLLVMGAALMCSGAMGLPVSGFPNMNAV
ALEDPTGVNYVDTIDFLKVGVPGSVLAYWIIVTVGYGIMRAVGM
>Symbiochloris_reticulata_Africa.PTC1
MKFTKELKYNAVEEWRAHYINYAAFKRLIYGEEKRKFGDNERMVPGTPQEDDHPTQEPLLHQTDDKAFMSLLDSELARVHEFY
LERERELGGQLDSLLSHARTVEVNERPATPSTEHGRRSSEGRLHLARRSSSRMQGALADLQAEAVSSEFWSQNQDFAVQAARE
QLRDDMQRLFVLLTGLERYIDLNIAGFRKALKKHDKVLADAESGKLKETYMPTVHRQCCLNKKPILEGALRKLQTLYAIVCCD
GNNEMALIDLKRRLGETVQFERNTVWKDMVQKDRKRGTLKVDDGLIGSWWHRARQPAAIAMSLAVFVVLLYTPTFREPEKRNC
LALLAFTSLLWCTEALPLYVTSMLVPLLVVVLRVLVDGSQHPPQRLSCKQAAPHIFHAMNSQVIMLLLGGFTIAAALSKHAIA
KILASWVLSKVGQRPGAVLMANMLVATFASMWISNVAAPVLCFSLVQPVLRTLDATHSFAKSLVMVALPVAFLGNFLVCGLLL
LVYQDPHFTEVRPMQPIKDPINGKQMYIIAVSVGSVTMWCFNSVLQQWFGEMGIIAILPMIAFYGFGILDKDDENSMLWNVVM
LAMGGLALGEAVTSSGLLLSIAEQLQHLVQGASVWRVLVIFCGLVLVATTFVSHTVGAMVVLPIIQSVGSQLSDPHPKLLVMG
AALMCSGAMGLPVSGFPNMNAVALEDSKGINYLTTIDFFKVGLLSSLIAYGLIVTLGYGIMYYGIGW
>Tetradesmus_deserticola.PTC1
MKFTHTLKYNSVPEWRESYINYSLLKKLILAASTAEYHEAYEGVHPAADLEDAGPRSPLLSRQASLQASLSRSLSVTMTREQR
EKEFLETLDNELAKIIRFYLKKEAEITAKYEEVSMMVQHAEGIASPTPGQAAEVSGLQAAQRTAFWSQSSRPVAAQREKLRAA
LEDLYATCCNLASYVEQNRTGFRKILKKHDKLVSHPMSAIYLPIVDQKFPESHAAHLRAQMDAIASLYSMVCCNGNADKAAAI
LRKQQQEQVFFERNSIWKDMVGQERRAATLHLQDGKEAVQESWLSTHRQAMLVTLALAVFAFLLYYPIFKEPEKQNCLALLAF
ASILWCTEAIPLFVTSMLVPFLIVLLRVLDDVDQEPPARLTPQQAAPRVFHTMFSQTIMLLLGGFAIAAALSKHFIAKQLAVA
ILSRVGRKPHHVLLANMLVATFASMWISNVAAPVLCFSLVQPILRTLPTNHAFCKSLVLGIALASNLGGMTSPISSPQNIFAI
ERMSMGGSPPSWLQWFAIALPVSFLGNVLCWAVILAVYKPGQNIKEVRPLKPNEDPMSGTQIYTIIVSLATVTAWCCNSFLQA
YTGEMGVLAIIPLVAFFGFGVLSKDDFNGFLWNVVMLAMGGLALGEAVQSSGLLATISNLISDLVGGQSLWAVLAIFCALVLV
GTTFISHTVGAMVILPIVQSVGDKMPGGHSKLLVMGAALMCSGAMGLPVSGFPNMNAVSLEDSTGQNYIGTADFLKVGVLGSV
LAYGIIISIGYGLMLAVGF
>Tetraselmis_striata.PTC1
MKFEHALEFNSVPEWRGHYLNYEQLKRLVYAVEAQQSAAQRASLDLSRRPSGVQEDPEAGSPLLPGGSEVEGGQEAEAEFVSC
AEGELKRVHAFLTAREAGLLGQWEEAALAAHSAEASYVPARTTRGGAFTRSHWWQQPTMQAQRRTLVATLGSLFVSLHDLSSY
AELNETGFRKILKKHDKVTGGALKGALLPVVQARLGAKRARLDQALEEVTSLYATLAFDGDADVAAAHLREGLREQVVFERSA
VWKDRMEEERRVATAHVVGPKAAAAKPWLLSGKAIAGLAALALAGAVLGSSAFGADDAGATKRACLAILLASAVLWCTEAVPL
YVTSMALIFAVVTLRAMLDGDGARLSAPDAMKRVFSKIFSQTVMLLLGGFTMAAALSKHLIAKRLAIGVMAQVGRRPASVLLA
AMGIALFSSMWISNVAAPVLCFSIVAPILRTLPTDDPLGAAMVIGIAMASNIGGMTSPIASPQNIFAIERMSMDGHPPSWLAW
FAVSMPVSITCLLLVWRLLLIIYPIDRDQEVRPLRQLDDPFTLHHAFVIAVCLATMGLWCANTWLLHLLGGMGVTALIPMVAF
FGFGTLGKDDFESFPWSVVMLAMGGIILGDAATESGLLAAMTEQIVGVVGSLTVCEVLVIFTGVIAVVTSFISHTVGAMVILP
VVQSIGAELAKSTGVDHSKLLVMGGALMCSGGMALPVSGFPNMSASSIQDPTGRNYVHVGDFLKTGIPSTAITWLCVIAIGYP
IMSAINL
>Trebouxia_sp..PTC1
MKFSQALKANSVPDWKHHYIHYSRLKKMIFRLEQLQGNAPLSPVPEHRQSLDFTNPSAPLLSRQSSSMLQRTSSGLEHAHIDE
LMFEREIHDELARVKAFYVEKHDELDAEVLAVLAKVAAAERRGISGPGHQDVEGGQSLPEEQRIAFWTDVNVPRNIKERLSGA
LTDVYIQLDNLSKFVELNYDGFRKILKKHDKMTNTELSGRLMPTVSDMLAKEQRKGALEGLKNSVVHEYALIAHSGGEREAEQ
ELGRHRRDQLDF
>Chlamydomonas_reinhardtii.PTC1
ATGAAGTTTACGCACCAGCTGAAGTTTAATAGTGTGCCGGAATGGAGAGAACACTACATACAGTATGGACATCTTAAGAAGTA
CATTTATGCGCTAGCTAAGAAGGAAGCGGACCTTCAAGCTGGCGGCCAAGATGAGGAGGCGCTGCTCGCCCCGCTGTTGGAAG
CGGAGCGTGATCAGGGCCCCACGGAGGAGGGCTTCCAGCGGGAGTTAGACGCGCAGCTTGCGGCCACGCTAAGCTTCTTCGCG
GTGAAGGAGGCGGACCTGCTCGCCAAGGTGTCCGCACTGGAGCTGGACATTCAAAGCCTGGAGAAGATCCCCAACCGCGCCGA
GGCGTCCACACTGGCGCGCATGGGCGGCAGCGCCAGCCCCGGCGGCCCCATGAGCAGCCCGCGCGCCGCCGCCGCCGCCGCCA
TGTCGGCCATGGCCTCGCTGGTCAGCCACAGCCCCTCCACACTGGACCTGGCGCGCATGGTCAACAGCACGCCGCCAGAGGAC
CACCGCAAGATCCGGGTCAAGTTCTGGGAGAACCCGCCCCGCCACCTGTTCAGCACCAACCTCAACACGCGCAGGGCCAAGCT
GCAGGCGCGCTTCCAGGACCTGTACATCTCGCTGCACGACCTGCGCGAGTTCTTGCACATCAACAAGGAGGGCTTCCGCAAGA
TCATCAAGAAGCACGACAAGCTGACCCGCGCCGTGGACCTGCGCGCCCGCTGGTGGCCCAACGTGGAGGCGCACCTGGCGCCC
GCCGCCAAGCAGGCCGAGCTGGACGGCGCCATCGGGGCGCTGACCGACCACTACGCAGTGCTGTACACGCGCGGTGACGTGGC
TCAGGCGGAGGAGCAGCTGTCGCGGGGGCTGCGCGAGCACATCACCGTGGAGCGAAACACCGTGTGGCGAGACATGGCGGCCA
TGGAGCGCAAGTACGCGGCGGTGTCGGTGAAGCAGGCGGCCGCGCCCGGGGCGCGCGTCACGTGGCTGCGCACGCACGCGCGC
TGGCTGAAGCTGGCCCTGAGTGTGGCGGTGTTCGTGGTGCTGGCCAATGTAGAGGTGTGGCCGGGCGCCGAGAACGAGCCGCG
CAACAACTGCCTGGCGCTGCTAGTGTTCGCGTCGCTGCTGTGGAGCCTGGAGGCTGTGCCGCTGTTCGTGACCAGCATGGCGC
TGCCGCTGCTGATTGTGGCGCTGGGGGTGCTGGTGGACCGCTCCAAGGACCCGCCGCAGCGCATGACCCCGCAGCAGGCGGCG
CCGGCCATCTTCCACGCCATGTTCTCGCAGACCATCATGCTGCTGCTGGGCGGCTTCGCCATCGCCGCCGCCCTGTCCAAACA
CGCCATCGCCAAGCAGGTGGCGGTGTCCATCCTGTCCCGTGTGGGCCGCAAGCCGCGCAATGTGCTGCTGGCGGCCATGTTCA
CAGCCACCTTCGCCAGCATGTGGATCAGCAACGTGGCGGCGCCCGTGCTGTGCTTCGGACTCATACAGCCCATCCTCAGGACG
CTGGACCCCGGCCACCCGTTCGCCAAGGCGCTGGTGATGGGCATTGCGCTGGCGTCCAACGTGGGCGGCATGACCAGCCCCAT
CAGCAGCCCGCAGAACATCTTCGCCATCGAGCGCATGAGCCTGGACGGCCGCCCGCCCTCGTGGCTGGCCTGGTTCGCGGTGG
CGCTGCCCGTGGCGGTCGCATGTAACTTTGTGTGCTGGGGTCTGCTGCTGCTGTGCTACCAGCCCGGCAAGGCCATCGCCGAG
GTGCGGCCCATCAAGCCCAACACCGACCCCATCAATGGCACACAGGTGTACATCATTGTTGTGTCGCTGCTGACGGTGGCGGC
CTGGTGCGCCAACACCTTCCTGCAGCGCTACACTGGCGAGATGGGCGTGATCGCGGTGGTGCCGCTGGTGGCGTTCTTCGGCT
TCGACGTGCTCAACAAGGACGACTTCAACAGCTTCCTGTGGAACGTGGTCATGCTGGCCATGGGGGGACTCAGCCTGGGCGAG
GCCGTCAAGAGCAGCGGCCTGCTGGCGGCGCTGGCGCTCACCATCAGCGACCTGGTCATGGGGCTCAGCCTGTGGCAGGTGGC
GGCCATATTCTGTGGCATGGTACTTGTGGCCACCACCTTCATCAGCCACACGGTGGGCGCCATGGTCATCCTGCCCATCGTGC
AGAGCGTGGGCGAGGCCATGGCCGGCACGCCGCACCCCAAGCTGCTGGTCATGGCGGCGGCGCTCATGTGCTCGGGCGCCATG
GGCCTGCCTGTGAGCGGCTTCCCCAACATGAACGCCGTGAGCCTGGAGGACAGCACCGGCAACGCCATCGTGGGCACCGGCGA
CTTCCTGGCGGTGGGCGTGCCCAGCTCCGTGTTCGCGTACGGCATCATTGTCTCGCTGGGCTACGTGCTCATGCTGGCGGTGG
GCTTCTAG
>Monoraphidium_neglectum.PTC1
ATGGACAAGGCTGAGAGGGAGCTGCGCAGTGAGCTGCGGGAGGAGGTCGGGTTCGAGCGCAACACGGTGTGGCGCGACATGGT
GGCCATGGAGCGGCGCACGGGGGCGGTCGTCAGGCAGGACACCCACGGCATCACAGACGACACCATCCGCGAGCCCTGGGTGA
AGCGCTACTGGCAGCCCATGACGCTGACTGTGTCGCTCATCGCGCTCGTCACGCTGCTGCTGGTGCCCATATTTGAGGACGAG
CCCGAGAAGCAGAACTGCCTGGCCCTGCTGGTGTTTGCGAGCCTGCTGTGGTGCACTGAGGCGCTGCCGCTGTTTGTGACGTC
CATGATCGTGCCGCTGCTGGTGGTGGTGCTGCGGGTGCTGGTGGACAGGACTGTCAGTCCGCCCGAGCGCCTGTCCCCGGAGA
AGGCCGCCCCCGCAGTGTTCCACATCATGTTTGGCCAGGTCATCATGCTGCTCCTGGGCGGCTTCGCCATCGCCGCGGCCCTG
TCGAAGCATTTCATAGCCAAGCAGCTCGCCGTCGCCATCCTGTCGCGCGTCGGGCGCCGCCCCCGGGACGTGCTGCTTGCCAA
CATGCTGGTGGCGACGTTTGCCAGCATGTGGATCTCAAACGTGGCCGCGCCGGTGCTGTGCTTCAGCTTAGTGCAGCCGATCC
TGCGCACCCTGCCGCCGACCCACCCCTTCGCCAAAGCCCTGGTCATCGGCATCGCCCTGGCCTCAAACCTGGGCGGCATGACA
TCACCCATCTCGTCCCCCCAGAACATCTTTGCCATTGAGCGCATGTCCATCGGCGGGGACCCCCCCTCCTGGCTCACCTGGTT
TGCGGTGGCCCTGCCCGTGGCGTTCTTTGGCAACGTGCTGTGCTGGGGGCTCATCCTCATAGTCTACAAGCCGGGGCTCAAAA
TCAAGGAGGTGCGCCCGCTGAAGCCCCCGGAGGACCCCCTCAGCGCCACCCAGATCTACGTGGTGGTGGTGTCACTGGCCACG
GTGGCCCTGTGGTGCTGCAATAACCTGCTGTCACACATCACGGGAGAGATGGGCGTGCTCGCAATCCTGCCGCTTGTCGCGTT
CTTCGGGTTCGGTGTGCTGTCGAAGGACGACTTCAACGGTTTCCTGTGGAACGTGGTGATGCTGGCTATGGGGGGCCTGGCCC
TGGGGGAGGCCGTCAAGTCCAGCGGGCTGCTGCTCACCATTGCACAGGCCATCCAGTCGATGGTGGACGGCCTCGACCTGTGG
AGCGTCCTGGCCATATTCTGCGCCCTCGTCCTCATGGCCACCACCTTCATCTCCCACACCGTCGGCGCCATGGTCATCCTGCC
CATCGTGCAGTCCGTCGGCCAGCAGATGCCGGGCCACCACGACAAGCTACTGGTCATGGGCGCGGCCCTCATGTGCAGCGGGG
CCATGGGCCTGCCGGTCAGCGGGTTCCCAAACATGAACGCGGTCGCGCTGGAGGACCCGACGGGGGTCAACTACGTCGACACC
GTCGACTTTTTGAAGGTCGGGGTGCCGGGGTCCGTCATGGTTTACTGGCTGATCGTGAGCGTCGGCTACGTGCTGATGCGGGC
GGTGGGGTACTGA
>Entransia_fimbriata.PTC1
ATGAAGTTCTCCCATGCCCTGAAATTCAATTCCGTGTCAGAGTGGAAGGCATTCTATATAGACTATGTACACCTCAAGAGGTT
TGTGTACCAACTGGAGGCCGCTGCGGTGACCGCCCTTCCAGAAACCCCGCGTCTGACTAGCTTATCTCTCGTGTCCACACTGG
GGGATGTCGAGGCAGGGGAGGAGGCATCGGGCCCTTCGCCTTTGGTCACAGATGCCGCATTCGTGCATGCCCTGGAGCGGGAG
CTTGAGAAGATAGTCACCTTCTACGCAAAGAAGGAGCAGGAGCTGGTCTCACAGATCGAGAATGCGGAGACGCAGGCGCGGGA
GTTTGAGGCGCGGCAGTACACCAGAAGCAGGCGCCAGGGGCACGGGCAATCCACGATGACGGCGCCGGAAGGGGGGAGGCGTG
TGGGCGATGAGGGAGCGGTGGATCTTCGAGAACCTCTGTTGGGAGGGAAGGAGAGCAGTGGACTGGGGAGCGGTGCCGGGCCT
GACTCCACAGCACAGCGCGTGTGGGTGTGGCAGCAGGAGGACAGTGGAGTGCGGCTGGCGAAGGAGAGGCTCAGGCTCCAGAT
GACGGACCTCTATGTCCAGCTGTTTGGCCTGCAAGATTACATCGACCTCAACCGCACAGGGTTCCGGAAGATCCTCAAGAAGC
ACGCCAAGGTGACGCACCATGCGCTGCAGTCGGAGTTCATGCCGCGGGTCAATGTGGGGCTGGACAAAAAACATGAGCAGCAG
CAGGAGGCTGCTCTGGCACGTGTGGTCCAGTTGTACTCCATCGTGTGCTGCAACGGAAGCTCGGAGCTTGCCCGGCTGGAGCT
CAAACGCCACCTGAGGGACCACCTGGTGTGGGAGAGGAACACTGTGTGGCAGGACATGGTGTCCAAGGAGCGCCGGAGTGCTT
CAGCTCACGTGTACGAGGAGAAGACAGCCTGGTACCTGCGCTGCCTGCGGCCGCTGTCGCTGCTGCTGGCCGTGGGTGTGCTG
GTCGCGCTTCTCGTGGTGCCCGTCTTCCCCGAGGCCCCCAAGCAGAACTGTCTGGCACTGCTTGCCTTTGTCTCTCTTCTTTG
GTGCACTGAGACCATCCCCCTCTACACCACCTCCATGCTCGTCCCCCTGCTGGCCGTCGTCCTCCGGGTGCTGGTTGACTCCA
AGACGGGCCGCCGGTTGAGCCCCCAGGAGGCAGCCTCAGCCATATTCCACACGATGTTCTCAGAGGTGATCATGCTCCTCCTC
GGTGGCTTTGCCATCGCGTCCGCCCTCAGCAAGTACTTCATCGCCAAGCGCCTCGCCACGACGATCCTGTCCCGCGTCGGCCA
GGCCCCCCAGAACGTCCTCCTCGCCTCGATGCTGGTCGCTTCCTTCCTTTCCATGTGGATCTCCAACGTGGCCGCCCCCGTGC
TCTGCTTCTCGCTCGTGCAGCCCATACTGCGGACGCTGCCCAGTGGCCACCCCTACGGCCGGGCACTGGTCATGGGCATTGCC
CTCGCATCAAACCTCGGCGGCATGGCCAGCCCCATCAGCAGTCCCCAGAACATCTTCGCTATCGAGGAGCTCTCGCTGCTCGG
TGACCCCCCGAGCTGGCTGCAATGGTTCGTCATCGCCCTCCCCATCTGCCTCGTCGGGAACATCGGGTGCTGGGCCCTGCTGC
TCGCCGCCTACGCCCCGCACAAGCAGCCCAACACGATCCGACCGCTGAAACCGACCCAGGATCCCATCACACTGTCCCAGGTC
TATGTTGTGGTGGTCAGTGTGGTCACGGTGGCCCTCTGGTGTGCCAACAGTTGGCTCAGGCAATACCTGGGCCAGATGGGAGT
TGTCGCTGTCGTCCCCCTCGTCGCCTTCTTCGGCACCGGGATACTCACCAAGGACGATTTCAACAACTTCTTGTGGAACGTCG
TGATGCTGGCCATGGGGGGCCTCGCCCTCGGCGAGTGTGTCACCAGCTCCGGCCTCCTCCACTCCATCGCCGCCTCCATCCAG
GCGACCGTTGCGAACCTGGGCCCGTGGCAAGTTGCGGCCGTGTTCTGCGGATTGGTTCTGGTGGCCACCACCTTCATTTCCCA
CACAGTCGGGGCCATGGTGATACTACCCATTGTGATGACCGTGGGGTCAGCCATGCCGGACCCCCACCCCAAGCTCCTAGTCA
TGGCCTCTGTGCTGATGTGCTCAGGCGCCATGGGGCTCCCAGTGAGTGGCTTCCCAAATATGAACGCCATTGCACTCGAAGAT
GGCACAGGGAAGACGTACCTGACCACCCTAGATTTCCTATGGGTTGGCATTCCAAGCTCATTGCTCGCATACGGGATCATCAT
CACCCTGGGTTACGGGATTATG
>Golenkinia_longispicula.PTC1
ATGAAGTTTACTCATCAGTTGAAGTTCAACTGTGCCCCGGAGTGGAAGGACTATTACATTCACTACTCCGTGTTGAAAAAGCT
CATATATCAGATAGGAGGCGATGATATACGTGAGAGTGCGGGTATAGGCCCTGCGGAGCAAGAGCCTCTGCTGCCAGTGATTG
ACAAAGACGAGAAAGAGAGACAGTTTGAGAGTCTCTTAAACACTGAGCTGACGAGGATCCTTGACTTCTACACCCGCACGGAG
CGTGAACTGTTTTCTCAGCTAGAGGGCCTGGGCCTTGAGATCAGAGAATATGAGGAGGGAAGGCTACCCACAACATCTGAAGG
GTTGGAAACAGAGCTGGATGGTCGCCGGCGCTTCTGGTCGTCCCACGACCTCCCCAAGGACCTCAAAGCCGCCAAAGACAGCC
TGGCCATGAAGCTTGAGGACCTGTACGAGGAGCTGAACAATCTGTTGGAGTACATCACGTTGAACCACACGGGCTTCAGGAAG
ATCCTCAAGAAACACGACAAGGTGACCCAGAGGGAGCTGAAGTCGCAGTACATGCCCCTGGTGGAGGCCAAGCTGGTCCATAA
CAAGAAGGAGAGAGTGCAGGAGCGCATCGACCAGGTGGTCCATCAGTATGCAGTGATTGTCTGCAATGGCCATGAAGGCCGGG
CCCTCACAGAACTCAGGAAGAAGTTGAGGGACCACCTTGTATTCGAGCGCCAGACTGTGTGGCAGGACATGGTGGCAATAGAG
CGTCGCACAGCTGCAGCACAGGTCAGCAACAAGGCGCCTGAGGGAGGCAAAGGCCCCCGTAAGTGGTGCCAGCGGTTGTGGAG
TCCCCTGGCCATAGGTGCCTCTTTGCTGGTGCTGGCTCTGCTGCTGAGGATGTCCCTCTTTGACGAGCCGGCGAAACAGAACT
GTTTTGCTATGTTGGTGTTCTGCTCCATGTTGTGGTGCTCAGAGGCGGTGCCCCTGTTTGTGACGTCCATGATGGTGCCCTTC
CTGGTGGTGGTGTTGAGGGTGCTGACAGATGGCACTCCTGAGCGGCAGAGGCAGACCCCCAACCAGGCAGCCGGGGCCATATT
CCATGCGATGTTCTCACAGACCATCATGCTGCTCCTCGGTGGCTTCACCATTGCGGCGGCTCTTAGTAAGCACTTCATAGCTA
AACAGTTGGCAGCGGCAATTCTCAGCCGAGTGGGTAGGAAGCCCGCAACAGTGCTGCTGACCAGTATGATGGTGGCGACCTTT
GCCAGCATGTGGATTTCCAACGTGGCGGCTCCGGTGCTGTGCTTTTCTCTGATGCAGACCATCCTGAGGACCCTTTCCCCCTC
ACACCCGTTTGCCAAGAGTTTGGTGCTGGGCATTGCCCTGGCCTCCAACATCGGGGGGATGACCTCGCCCATTGCCAGCCCAC
AGAACATCTTCGCAGTAGAGAGGATGTCACTCCATGGCAGCCCCCCGGGGTGGTTGAGTTGGTTTGGTGTCGCCATTCCTGTC
AGTATCTTGTCCAACCTGCTTGTTTGGGCCGTTATCTTGGTCGTGTACAGGCCAGGCCGCTACATCAAGGAGGTAAGGCCCCT
CAAGGCCCCCGAAGACCCCCTCACAGGTACCCAAATGTACGTCATCGGGGTCAGTCTGCTCACTGTGGTGCTCTGGTGCTGCA
ACTCCTTCCTCCAGAAATATACCGGAGAGATGGGGGTGCTGGCCCTACTGCCCATGATTGCCTTCTTTGGCTTTGGTATCTTG
AACAAGGACGACTTCAACGGCTTTCTTTGGAACGTGGTGATGTTGGCCATGGGGGGTCTGGCCTTAGGCGAGGCAGTGAAGAG
CAGCGGTCTGCTGGCCTCCATTGCTCAGGGCATTGAGGAGCTGGTGCAGGGTATGAGCTTGTACCAGGTGTCTGTGGTGTTTG
GCCTCATGGTGCTGGTGTGTACCACCTTCATCAGCCATACGGTGGGGGCCATGGTAATCCTGCCCATTGTGCAGTCCGTGGGG
GAGAGGATGTCAGATCCCCATCCCAACGTATTAGTCATGAGCGCTGCACTGTTGTGTAGCTGCGCGATGGGTCTGCCGGTC
>Oedogonium_cardiacum.PTC1
GAGGCAATCCCCCTCTTTGTGACCAGCATGCTCGTGCCCGTGTTGGCCGTATCCCTGCGGGTGATAGTGGTGGACGGAAGGAG
GCTGGAGCCGCCTGACGCGGCCTCATACTTGTTCGGGAAGATGTTCTCACAGGTGGTCATGTTGCTGCTCGGTGGTTTTGCCA
TTGCTGCAGCTTTGAGCAAGCATAACATTGCGCGCAAGATGGCCATAGCCGTCCTTTCAAGAGTTGGGCGCGAACCTGCACAC
GTTTTGCTAGCCACAATGATGGTTGCCACTTTCCTATCTATGTGGATTTCAAATGTGGCTGCACCAGTGCTTTGTTTCTCTCT
CGTGCAGCCCATCTTGAGGACACTGGATACAAACCATAGCTTTGCGAAGGCGCTGGTGATGGGCATCGCGCTAGCTTCCAATG
TGGGCGGCATGGCCAGCCCCATCAGTAGTCCCCAAAACATATTCGCCATCCAAGTCATGGGCAGTGGGGGTGACAAACCTCCA
AGTTGGACGCAATGGTTTGCTGTGGCATTGCCAGTGTCCGTCGTGTGCAACATGCTCATTTGGGCACTGCTATTGCTGGTGTA
CCAACCACATAAGCACATCAAAGAGGTTCGCCCCATTCGTGCAATCCAGGACAAGTACACTCTTCAGCAAGTCATGGTGGTTC
TGGTCAGTCTGGTTACAGTAGCTCTGTGGTGCCTGAATGGCGTGCTGGAGCCCTACCTGGGATCTATGGGTGTCATAGCTATC
TTACCGCTGGTGGCGTTCTTCGGTTTTGGCATCTTGACCAAGGATGATTTCAACGCGTTTTTGTGGCATGTGGTCATGTTGGC
CATGGGGGGACTGGCAGTGGGAGAGTGTGTGAAGAGCTCACACTTATTGCAGACCATTGCTGCCGAAATCCAGGAGATGACGG
TTGGATGGTCCATGTATGCTATTGTGTGCATGTTTTGTGCGCTGGTGCTATGCTGCACGACCTTCATTTCGCACACGGTGGGC
GCCTTCGTGATTTTGCCTGTTTTGAAGAGTGTGGGAGACGAGATGGCGAGATCCGGGCAGCCAAACCACTCCAAGCTTTTGGT
CATGGCCGCAGCTCTGATGTGCTCTGGTGCCATGGGCCTGCCAGTGAGTGGTTTCCCCAACATGAACGCGGTGGCCCTGGAGG
ACCAAGCGGGGCTCAACTACGTAGCCACAATAGACTTCATCAAAGCAGGGCTGCTCAGCTCAGTGTTTGCATACGTCATTATT
ATAACCCTGGGATACTCACTTATGTTTATGATCGGTTTT
>Cylindrocapsa_geminella.PTC1
ATGAAGTACGGCACGCAGCTTAAGCTGAACATGAATCCTGACTGGAAGGATCACTACATCAACTATTCCATGCTGAAGCACAT
GATCTATCAGATCTCCAATAAGGAGAACCAGGTGCCTATTGACTTTGAGCAGGGAGAGCCAGCAGAGCCCCTACAGCCCCTCC
TTCGATGGAACAGCGCGCCGGACCTGGAGAGCACGTTCGTAAAGGCATTTGAAGGGGAGCTCGCGCGTGTGATTGAGTTCTAC
ACGCAGAAAGAGGCAGAGCTCTTCGCCAAGTGCGGTACTCTAGGTTTGAAGATTCACCAGATGGATGGGCTGAGCGCCCCGCC
GTCGGACTCTCCAGTCGCTTCTCAGGCCGAGGACGAGCGCATCGCGTTCTGGGCGCACGTGCCGGGCCACTTGGTCGCCCAGC
GCGACGAGCTTCGCAAAGAGATGGAGGCGCTGTACGTGGCCCTTAAGGACCTGGAAAGCTTCAGGTCGCTGAACTTCGAGGGC
TTCCGCAAGGCTCTGAAAAAGCACGATAAGGAGACCACCACTGCGCTTATGCCGCAGCTGATGCCCGTGCTGCAGTCCAAGCT
GTCCTCGTCGCAAACGGCCATCATCCAACAGCGCGGAGACGTGGTGCTGCACCTGTTCGCTGTGGTTGTGTGCCATGGCGACC
TGCAGGCGGCTCGCAGGGACCTCGACTCGCGGCTGCAGGATGAGGTTCTGTTCGAGCGACGCAGCATCTGGCAGGATATGGTG
GCGACCGAGCGGCTGCGTGGCTCAGCGCCGCAAGTGGTGCCCAAGGACTCCGGATCGGCGTACGGCGCGAGCCTGACAGGCTG
GTTCGAGCGGAACAAGCAGTGGACACTGCTGCTGCTGTCGTTCGGGGCGTTCGCGGTGCTGCTGAACTACCCCGTGTTCGACG
ACGAGTCCAAGAACAACTGCCTCGCGATGTTCGTGCTGCTGTCTCTTCTGTGGAGCACAGAGGCGCTCCCACTATTCGTCACG
AGCATGCTGGTGCCGCTGCTGGTGGTCATACTCAGGGTGCTGGTGGACAACACCCAGGACCCGCCCGTGCGGCTGACGCCTCA
GGACGCTGCGGCGTCCATCTTCCACTCGATGTTCTCGCAAACCATCCTGCTGCTGCTCGGCGGCTTCACCATCGCTGCCGCGC
TGACCAAGCACTTCCTGGCCAAGCAGCTGGCGGTGACCATCCTCTCACGTGTCGGCCGCAAACCCGAGCACGTGCTGCTGGCC
AACATGCTGGTAGCAACGTTCTTGTCAATGTGGATATCGAACGTGGCGTCGCCTGTGCTGTGCTTCTCGCTGGTCACTCCGAT
CCTGCGCACGCTGCCCACGCACCACCCCTTCGGCAAGGCGCTCGTTATGGGGATCGCGCTCGCGTCGAACCTCGGGGGGATGA
CCAGCCCGATCAGCAGCCCTCAGAACCTGTTTGCGATCGAGCGCATGGGTCTGGATGGTCACCCGCCCAGCTGGCTGGCGTGG
TTTCTGGTGGCACTGCCCGTGTCCTTCTTAGGCAACCTGATCTGCTGGGGCGTGCTGCTGGCAGTGTACCGCCCGCAGCAGAA
GATTCGAGAGGTCCGTCAGCTGAAGCCCAGCGGCGACCCCATCACGTGGAAGCAGGTGTACGTGCTTGTCGTGAGCCTCACCA
CAGTGGGCCTGTGGTGCGCTAATACGGCCTTGCAGCAGTACACGGGCGAGATGGGCGTGCTGGCGATCGTGCCGCTGGTGGCG
TTCTTCGGTTTCGACATCCTCAACAAGGACGACTTCAACCACTTTCTGTGGAACGTGGTGATGCTGGCGATGGGCGGCCTGGC
GCTGGGCGAGGCAGTCAAGTCGTCCGGCCTGCTGCTGACCATCGCCGAGGCGATCAAGGAACTCGTAATCGGGTTCGATCTCT
GGACTGTTCTCGTGATCTTCTGCGGCCTTGTTCTCGTGGGCACCACCTTCATCTCGCACACCGTCGGCGCGATGGTGATCCTC
CCGATCGTGCAATCCGTGGGCGACCAGCTGCCCGGCCCGCCGCACGCGAAGCTTCTCGTGATGGGTTCGGCCCTAATGTGCTC
CGGCGCAATGGGCCTGCCCGTGAGCGGCTTCCCGAACATGAACGCGGTGGCACTGGAGGATCCCACGGGGCTGAACTACGTGG
CCACCATCGACTTCCTCAAGGTCGGCGTGGTCTGCTCGTTCTTTACGTACGGCATCATAGTAACGCTCGGCTACGGCCTCATG
CTGCTGGCCGGCTTC
>Scherffelia_dubia.PTC1
CTGAACGTGACGGGGTTCCGCAAGATCCTGAAGAAGCACGACAAGGTGACGAACAAGGAGCTCAAGGGCGACTTCCTGCCCAT
CGTGGCCAGCAAGCTCAACCCCAAGAAGGAGCGCGTGGACCAGGTGCTGCAGGAGCTCATCGGCGTGTACGCGACGATAGGCT
GCGAGGGCGATGTGAACGCCGCAGCCGCGCAGCTGCAGGTCAACCTGCGCGACCAGGTGGAGTTCGAGCGGCAGGCGGTGTGG
AAGGACAAGATCGAGGAGGAGCGCAAGCTGGCCAACGCCAAGGTGGTGGACAAGGGCAAGAAGGCATGGTACGCGGACTACAA
GAAGCCCTTCTGCCTGCTGCTCAGCGCCGCGCTCATCTTCGGGGTGCTCGGCTCGCCCCTCTTCCCCTCCTCCCCCCCCAAGC
GCAACTGCCTCGCCATCTTCGTGGGCGCCGCCGCGCTGTGGTGCACCGAAGCGGTGCCGCTGTACGTCACCTCCATGCTCATC
CCCGCCGCCGTCGTCACGCTGCGCGCGCTCGAGGACGCGGACGGCGTGCGGATGACTGCCACAGAGACCGCGGACCGCGTGTT
CTCAAAGATGTTCAGCCAAACCATCATGCTGCTGTTGGGGGGGTTCACCATGGCAGCTGCCATCACCAAGCACCTCATCGCCA
AGCGGATGGCCTTTCAGGTGCTCTGCCGCGTGGGGCGGCGGCCGGGGAATGTGTTGCTGGCCTCTATGTTCATTGCCCTCTTC
AGCAGCATGTGGATCTCCAACGTGGCCTCGCCCGTCATGTGCTACGGCATCGTGCAGCCCATCCTGCGCACGCTGGCGCCCGA
CGACCGCTTCGCGTCTGCGCTGGTGATGGCGATCGCGCTGGCGGCCAACGTAGGCGGCATGACCTCCCCCATCGCCAGCCCGC
AGAACATCTTTGCAGTGGAGCGCATGGCGATGGATGGCAACCCGCCCAGCTGGCTGGCCTGGTTCTCGGTCTCCATGCCCGTC
TCCATGTTCTGCATCCTCGTGCTGTGGCGCATCATCCTCGCGGTGTACAGGATCGGGCCGCACACTGCGGAGGTGCGCCCCAT
GCGGCCGCTGGACGACGTCTTCACGATGCAGCACGTGTTCATCATTCTCATCAGCGTCCTCACGATGGGCCTCTGGTGCGCCA
ACACGTGGCTGCTCAGCGTGCTCGGCGGCATGGGCATCACCGCGCTGCTGCCGATGGTGGCATTCTTCGGCTCGGGCATCCTC
AACAAGCTCGACTTCGAGAGCTTCCCCTGGAGCGTGGTGATGCTCGCCATGGGCGGCATTGTGCTGGGGGAGGCGGTCAAGAG
CAGCGGGCTGCTGGGCTTCATCGCGCAGGGCATAGTGGGGCTGGTGGATGGGTTCACAGTGTGGCAGGTGCTGATGATCTTCG
GCGCGGTGATCGGGGTGGTCACCAGCTTCATCTCGCACACTGTGGGCGCGATGGTCATCCTGCCGGTGGTGCAGAGCGTGGGC
GAGGAGATGGCCAAGGCCTCGGGCGTGGCGCACTACAAGCTGCTGGTGATGGGGGCGGCGCTGTGCTGCTCGGGTGGGATGGC
GCTGCCGGTGAGCAGCTTCCCCAACATGACAGCCGCCTCGCTGACAGACCCCACC
>Hafniomonas_reticulata.PTC1
ACTGCTGGCAATGCCGAGAAGGCTGAGGGTGAGCTGAGTGCTCATCTAAGAGAGCAAGTGCAGTACGAGCGCAATACAGTGTG
GCGTGACATGGTGGCGAACGAACGCACGAAGACCGCCGAAAGCAGCAAGCAAGTTCGAACGCTATCGCAATCTATTGTCTGCA
TCGCCATCGTCGCTGTTGTCGCTCTGATCCATGCCAACATCTTCCCAGATCAACCCTCCAAAAACAACTGCCTTGCGATGCTC
GTGTTTGTGAGCATCCTATGGGCATCCGAAGCAGTTCCTCTGTTTGCGACGTCTATGATGGTGCCTTTGCTTACGGTGCTTCT
TGGCATTTGGGTGGACCCTTCTCAAAAACCACCTAAGAGGCTGGACCACACGGCTGCTGCTTCTGCTGTTTTCTCTGCTATGT
TCAACCAGGTCATCATGCTTTTGCTCGGTGGCTTTGCCATCGCCGCCGCCCTCTCAAAACACTTCATCGCGAAGCGTGTTGCT
GTCGCCATTCTCTCCCGTGTGGGTCGCAAACCTCACAACATCATCCTCGCGTCTATGTGTGTGGCGGCCTTCATGTCGATGTG
GATATCGAACGTCGCGGCGCCTGTGCTTTGCTTTTCGTTGATAGCGCCTATCTTGCGAACGTTGGATGCATCGCACCCCTTTG
CCAAGGCGTTGGTGATGGGCATTGCTTTGGCGTCGAATGTGGGAGGGATGACGAGCCCTATTAGCAGCCCCCAGAACCTGTTC
GCGATTGAGCGCATGTCCATGCCCCCTTCCTGGTTGTCTTGGTTTGCGGTTGCGCTTCCAGTGGCAGGTATTTCTATCATTCT
CTGTTGGCTCATCATTCTCATTGTATACCGGCCGTGGACAAAAGTGAAGGAAGTCCGACCCTTGAAACAAGATGATCCTATCA
CGTACACGCAGGCATTCGTCGTTCTCGTCAGCGTCGTCACGGTTGGTCTTTGGTGTGCGGAGTCTCAGCTAGAAAGTGTGTTT
GGACAGATGGGGGTGATTGCAATTTTGCCCATGCTCGCATTCTTTGCAACTGGTATTCTCAACAAGGATGACTTCAACGGTTT
CTTGTGGAACGTGGTTATGCTTGCCATGGGCGGGTTAGTGCTAGGTGGAGCTGTGAAGAACTGTGGATTGTTAGAAGACATTG
CAAACGCTATCAAGAACCAAGTTGCTGACTTGGAATTGTTCCAGGTTCTTGTCGTGTTCTGCCTGCTCGTGCTCTTCTGCACG
ACATTCATTTCCCATACAGTGGGCGCCATTGTCATTCTACCCATCGTTCAGCAAGTGGGCGAAAGTTTCCCTGGCACCCCTCA
TTCAAAGTTGCTGGTCATGGGCGCCGCCCTCATGTGCAGTGGTGCTATGGGCCTACCCGTGTCAGGCTTCCCAAACATGAATG
CGGTCGCACTGGAGGACGCCACCGGACAGACGTACGTGCAGACTCTTGATTTTCTCAAAGTCAGTGTTCCTTGCTCAATCTGT
GCG
>Tetraselmis_chui.PTC1
CTACTGAGCGCAGCCGTGCTTTGGTGCACGGAGGCCGTCCCGCTTTACGTCACGTCGATGGCGCTCATCTTCGCTGTGGTCGT
TCTGCGGGCGATGTTGGACGGGGACGGCGTGCGGATGTCAGCGCAGGACACCATGAAGCGCGTATTCTCTAAGATCTTCAGCC
AGACTGTCATGCTTCTCCTAGGGGGCTTCACTATGGCGGCAGCCCTGTCGAAGCACCTGATCGCCAAGCGGTTGGCCATCGGG
GTGATGTCGCAGGTGGGCCGTAAGCCATCCAACGTGCTCATCGCCTCCATGGCCATCGCACTCTTCAGCAGCATGTGGATCTC
CAACGTAGCGGCGCCCGTACTGTGTTTTAGCATAGTGCAGCCTATCCTGCGTACTCTACCTACGGATGACCCTCTAGGTGCTG
CGCTGGTTATGGGTATCGCGATGTCCTCCAACATCGGCGGCATGACCTCCCCCATCGCCAGCCCTCAAAACATATTCGCCATC
GAACGCATGTCCCTGGACGGCCACCCTCCTAGCTGGATTACCTGGTTTGCCGTCTCCATGCCCGTGTCTATCACCTGCATGCT
GCTCGTATGGAGACTCCTCCTCGCCATTTACCCGGTCAGTAGCAATCAGGATGTCCGTCCGCTGCGGCAACTACACGACCCGT
TCACGTTTCAGCACGTGTACATAATCATCGTGTGCTTA
>Volvox_globator.PTC1
CTGTGGTCGTTGGAGGCCATTCCCCTGTTCGTCACCAGCATGGCTCTTCCGCTCCTCATCGTCGTGCTGGGGGTGCTACAAGA
CTCACCCAACACAGAGAAACCTGCCACTCGACTCACTCCGCAGCAGGCGGCGACCGCCATATTCCATGCCATGTTCTCACAGA
CTATCATGCTGCTGTTGGGCGGTTTCGCTATCGCTGCGGCGCTCTCCAAACATGCCATTGCCAAGCAGGTTGCGGTTGCCATT
TTGTCCAGAGTGGGTCGTAAGCCTCGCAACGTGCTGCTGGCCGCCATGTTCACCGCCACGTTCGCCTCCATGTGGATATCGAA
CGTGGCAGCACCCGTGCTGTGTTTCGGATTGGTACAACCCATTTTGCGCACCCTGGACCCTGGACACCAATTCGCCAAGTCTC
TAGTCATGGGTATCGCCCTCGCCTCCAATGTGGGTGGCATGACCTCCCCCATCTCGTCGCCGCAGAATATCTTCGCGATAGAG
CGCATGTCCTTGGACGGCCAACCGCCTAGCTGGCTGTCCTGGTTTACGGTGGCGCTACCCGTGTCGATTGCAGCCAACATGCT
CTGCTGGGCGATCCTGCTGGTGGTGTACCAACCCGACCGAGCCATCGCGGAGGTTCGGCCCATTAAACCCAACACCGACCCCA
CCAACGGCACCCAGGTGTACATCATCGTTGTGGCGCTGCTCACGGTGTCAGCATGGTGTGCAAACACATTCCTACAGAGCTAC
ACGGGTGAGATGGGTATCATCGCCATCTTGCCCCTTGTGGCGTTCTTCGGGTTCGATGTGCTCAACAAGGATGACTTCAACTC
CTTTCTGTGGAATGTGGTCATGCTGGCCATGGGGGGACTCGCGCTGGGTGAGGCCGTTAAGAGCAGTGGCCTGCTGGCCGCCC
TCGCCTCGGACATCAGCGACCTGGTGTTGGGTCTCAGTCTCTGGCAGGTTACACTCATCTTCTGTGGACTGGTTCTGGTGGCC
ACCACTTTCATCAGCCACACGGTGGGCGCCATGGTCATCCTACCCATCGTACAGTCCGTGGGGGAGCGAATGATTGGCACACC
GCACCCCAAACTGCTCGTCATGGCGACAGCACTGACATGTTCAGGTGCCATGGGTCTGCCTGTGAGTGGCTTCCCCAACATGA
ACGCGGTGAGTCTGGAGGACGCCACGGGCAACCCCATCGTCAGCACAAAGGACTTTCTCATGGTGGGGGTGCCCTCGTCCATT
GCGGCGTACGGCATCATTGTGACGCTGGGGTACGGCTTGATGCTGTTGGTGGGCTTT
>Volvox_aureus-M1028.PTC1
ATGAAGTTCACACATCAGCTGAAGTTTAACAGCGTCCCGGAGTGGCGCGAGCACTACATACAGTATGCACATTTAAAAAAGTA
CATATATGCTCTTGCAAAGAGGGAGGCGGATTTGCAGGCTGGGGGCCAGCTTCATGATGACGAGTTGCTCACCCCACTTGTTC
CGGAGACTTCACGCCAGGGCTTTAGCGAAGAAGGCTTTCAACGGGAACTTGATGCCCAGCTTGCTTCAATTCTTTCATTTTTT
GCTGTCAAAGAAGCAGAGCTGCTTGCTAAAGTGTCGGAACTGGAATTAGACGTGCAAAGTTTGGAAAAAATTCCGAATCGGCA
AGAAGCATCCTCGATGTCTCGCATTAGCGGTAATCCATCGACTACCGGCTATCACAGCAGCAGCAGTCCTAGGGGGCCTGTCG
GTTTGCCGTCGATGTCGTTGATGTCCGTTTCTCCCTCCACGCTTGACCTGGCACGGATGGTGGACTCTACTCCGCCAGAGGAC
TTCCGCAAAGTGCGAGTCAAGTTCTGGGAAAATCCGCCACGGCACGTTTTCTCTCCAAACCTTAACACGCGACGGCAAAAGCT
TTTAGGGCGGTTTCAGGACCTGTTCATTGGCCTGCACGACTTGCGGGAGTATTTGCACATCAACAAGGAAGGATTTCGGAAGA
CTATCAAGAAGCACGATAAGCTGACTCGCTCTGTCGATCTTCGCGTGCGCTGGTGGCCGAACGTGGAAGTGCACTTGGCGCCC
GTAGCGAAGCAACAAGAGCTAGAGAGGGCAATTGCGGCGCTCACGGATCACTATGCGGTGCTGTACATGGGGGGTGACCTTAC
AAAAGCCGACGAGCAACTGTCGCGAGGCCTGCGTGAGCATATCACGGTGGAGCGCAATACTGTGTGGCGCGATATGGCGGCAA
TGGAGCGCAAATACGCGGCGGTGTCTGTCAAACAGGCGACAGCTCCCAGAGATGGTGGGCGGCAAGCTCATGTCCGGTGGGCT
AAGCTGGCCGCGTGCTGCCTGGTCTTCGCATCGCTGTTGCTATGGGGGGGACCCGTGGAGAATGGGCAGGTCAACCAGCCCAG
AAACAACTGCTTGGCGTTGCTGGTGTTTGCATCTTTGTTGTGGTCACTGGAGGCCGTGCCTCTGTACGTCACAAGTATGGCTC
TACCTTTTCTGATTGTCGCCATGGGTGTCCTTGTTGACCACCCAAGTGATTCCAAGGATCCACCTAGACGCCTCACACCGCAG
CAAGCTGCACCAGCGATCTTCCATGCCATGTTCTCACAGACAATCATGTTACTTCTTGGTGGTTTTTCGATAGCGGCCGCTCT
CTCCAAGCACGCCATCGCAAAGCAGGTGGCTGTGGCCATTTTATCTCGTGTTGGAAGGAAACCACGCAACGTACTGCTTGCTG
CAATGTTTACAGCCACATTCGCATCAATGTGGATTTCCAATGTGGCGGCACCAGTACTCTGTTTTGGCCTAATTCAACCCATC
CTGAGAACCCTGGACCCGGGACATCCTTTTGCGAAGTCCCTGGTTATGGGCATTGCGCTGGCTTCCAATGTCGGAGGAATGAC
ATCGCCCATATCTTCACCCCAAAACATTTTTGCAATAGAGCGCATGTCGATGGATGGACAGCCGCCCAGCTGGCTCTCTTGGT
TCGCTGTAGCAATACCAGTGTCCATCACATGCAATTTTCTCTGTTGGGCCTTACTCCTTCTGGTCTACCAACCAGGACGAGCT
TTGGGTGAGGTCCGACCGCTAAAACCCAACACGGACCCCATTAACGGAACACAGGTGTACATCATTGTTGTGTCGATGTTGAC
TGTGGCAGCCTGGTGTGCGAACACATTCCTCCAGAGGTATACTGGCGAGATGGGCATCATCGCCATTGTCCCTTTGGTAGCTT
TTTTCGGTTTCGACGTACTCAACAAGGATGATTTCAACTCGTTTCTATGGAACGTCGTCATGCTGGCCATGGGCGGGTTGTCT
CTAGGTGAAGCAGTTAAGAGCAGCGGTTTGCTTGCAGCACTCACAAACAGTATTAGTGAACTGGTGACAGGCTTCACTATGTT
CCAAGTTACCCTCATATTCTGTGGCCTGGTGCTGCTGGCGACAACGTTTATCAGCCACACAGTAGGGGCTATGGTCATCCTAC
CAATCGTGCAGAGTGTTGGAGAGAGCATGGCTGGGACACCACACCCTAAGCTGTTGGTCATGGCATCGGCGCTCATGTGCTCT
GGTGCTATGGGCTTGCCTGTCAGCGGATTTCCGAACATGAACGCTGTCAGCTTGGAGGATAGCACTGGCAATGCAATTGTCAG
TACTAAGGACTTCCTGTACGTCGGTGTGCCTTCATCGGTGATGGCTTATGGCATAATCGTCAGTCTAGGGTATGTGCTCATGC
TGTTGGTAGGTATG
>Ignatius_tetrasporus.PTC1
GACATGTACTCCAAACTGGAAGATCTGGTCAACTTTCTCGAACTGAATCGTGAAGGCTTCAGGAAAGCATTGAAGAAGCATGA
TAAAGTGACTCAACGCAATCTGTCTCCAATCTTGCTGCCGGAAGCTTTGGAGGCACTCAATGTGCAGGACAACAAGAACGCAA
TTGAAGAAAGGAAGCAGGATGTCGTGCAGTGTTATGCAACAAGTCAGCAGAAAGGCGAAGTGCTTGCCGCAACCAATGTACTC
AAACGCCAGCAAAGGGAGATGGTGGAGTTTGAGCGCAGCACGGTATGGCGGGAGCACATGGCAGTGGAGCGGCAGCACGCGCA
AGCGACCGCGAAAGCCGTGGAGTTGACAGGCTGGCAGCATTGGTGGAGCAGCCATCGCAATCTGATTTGGATCGCGGTTGCTG
TCGCCGCGTTCTTCATTGTGCTGCTTGTTCCCATGCCCATCTTTGACACCGTTGAACAGCACAACTGCGCTGCTTTGCTAACT
ATGGCGGGCATTCTATGGTGCTTCGAAGCGCTGCCGCTGTTCGCAACGGGCATGCTGGTACCGTTTCTTGTTGTTGTGCTGCG
CGTGATGCGCGTTGCCGACACACCACAGCACGCTTGCACGCATGACTGCCGGCTGTCGGCGCCTGATGCTGCGAGCGCTGTGT
TTCACAGCATGATGGACCAGGTGATATTTTTGCTGTTGGGAGGCTTTACGATAGCAGCAGCGCTGACGAAACACAACATTGCG
AAGCAACTCGCATCAGCAGGATTGGCAAGTGTGCGCGACGCGCCTGGCAAACTGTTGTTCGCAGCAATGAACATTGCGACAGT
GTCGAGTCTTTTCATCTCCAATGTTGCGGCTCCTGTGCTCTGCTTCTCGCTCGTGCAGCCGATTTTGCGCACGCACAAGCACG
ATCATCCGTTTTCCAAGGCGCTGGTTATCGGCATCGCGCTTGCATCAAATATTGGCGGCATGACCTCCCCCATTTCAAGTCCG
CAGAACATATTTGCGATTCAGAAGATGGATGAAGACGGACGAGCACCAAGCTGGCTCGTTTGGTTCGCAGTTGCGCTGCCTGT
CGCGTTTGCATGCAACGCCGCGTGCTTCACAGTCATCTGGCACTTCTACAAACCCTACAAAACACGCACGGCTATTCGTCTCC
CGAAACTTACCGACAAACTGAACTCCACTCAGGTGTTTGTGATCGTCGTGAGCTTGCTGACGGTTGGATTGTGGTGCGCGAAC
GCGCAGCTCGAAAAGTACTTTGGCAAAATGGGAATCATTGCGATCCTGCCTGTTGTGTTGTTTTATGGAAGTGGCGTTTTAAA
CAAAGACGACTTGAACAACTACTTGTGGAACGTTGTCGTGCTGGCAATGGGTGGGCTCGCGCTTGGAGAGGCTGTGAAGTCAT
CGGGCCTGTTACACGTCATCGCGAATCTGTTGGCGGATGCTGTTGGATCGTTGGACCTGTGGCTCGTGCTCATCGTGTTTTGT
GGGTTTGTGCTGATCGGTACCACTTTCATCTCCCACACGGTGGGAGCGATGATCATCTTGCCCATCCTTCAAACAGTTGGCAA
GAATCTTCCAGGCGCGCCGCATCCGCAGCTGTTAGTCATGGGAGCCGCATTGATGTGCAGCGGCGCCATGGGTTTGTCCGTCA
GTGGCTTTCCAAACATGACTGCAGCTGCTCAACAAGCCAGCACCGGCGAGCATTACATCAATTCAGCTGATTTCCTGCAAGTT
GGCATTCCGTGTTCCGTCATTACATATGGCATCATCGTTACTCTTGGGTACGGTTTGATGCTGGCTGTGGGACTG
>Gonium_pectorale.PTC1
ATGAAGTTTACCCACCAGCTGAAGTTCAACAGCGTCCCGGAATGGCGTGAACACTACATACAGTATGCGCATCTTAAGAAGTA
CATATACGCGCTTGCGAAGAAGGAGGCCGATTTGCAGGCCGGAGCGCCGACAATTGAAGAAGGTCCGCTTGCACCGCTGTTGC
AGGATGCGCGTGCTACACAGGGCCCCTCCGAGGAAGGCTTTCAGCGCGAGCTTGACGCTCAGCTGGCAGCTCTCTTGGCCTTC
TTTGCCGTTAAAGAGGCGGATTTGCTTGCGAAGGTGTCCGGGCTGGAACTGGACGTGCAGAGCTTGGAAAAGATTCCTGGTCG
TCGCGAAGCTTCAACATTATCACGTCTGGGGATAACAGGTGGCCCATGCAGCAGCTCAGATGCCATTGCCCCTGGTGCCGCGG
TGGGCGCGACTGTAGCGCCCTTGACCACACTGACAATGGACGCGAGCCCGTCCACCCTTGACCTGGCAAGGATGGTCACATGC
ACGCCGCCTCAAGACCATCGCAAAGTGCGCGTCAAGTATTGGGAAAATCCACCTCGGAGCACATTCTCGCCTAACCTCAACTC
GCGGAAAATGAAGCTGCAGGGACGCTTCCAGGACCTGTACATTGGGTTGCACGATTTGCGGCAGTTCCTTATTATTAATAAAG
AGGGCTTTCGCAAGATCATCAAGAAGCACGACAAGCTAACCCGCATGGTGGACCTTCGTGATTGTTGGTGGCCAAACGTAGAG
GCGCACCTGGCCCCAACCACCAAGCAGCAGGAACTTGATCGAGCTATCGCCGACATCTCCGACCACTACGCCGTTGTTTACAC
CGGAGGCGATGTGGCCAAGGCGGAGGAACAGCTTTCCAGGGGCCTCCGTGAACACATCACTTTTGAACGAAATACAGTCTGGC
GTGACATGGCGGCCATGGAGCGCAAGTACGCCGCTGTGTCAGTCAAGCAAGCTGCCCCACCGGGTGGTGCCAAGGCCAGCCGG
CTGCGCGACTACCTTCAGTGGACAAAGCTTGCACTGTCGTGCGCAGTGTTTGCAATCTTGCTGAACGTCGATGTGTGGCCAGG
GCCACAAAACGGCCCGCGGAACAACTGCCTGGCACTACTCGTTTTCGCCTCACTTCTATGGTCATTAGAGGCCGTCCCGCTCT
TCGTGACAAGTATGGCCATCCCTTTTCTGGTTGTGACGTTAGAGGTTTTGACAGACGGGACGAAGGATCCACCGCAGCGCCTG
ACTCCTCAGCAAGCGGCATCTGTCGTTTTCCATGCAATGTTCTCACAGACCGTTATGCTCCTGCTGGGTGGCTTCTCTATTGC
GGCTGCGCTGTCGAAACATGCAATTGCTAAACAGGTGGCAGTTACGATCTTGTCCCGAGTCGGGCGCAGACCACGCAACGTCC
TTCTTGCTGCCATGTTCACCGCGACATTTGCCTCAATGTGGATATCGAATGTGGCGGCACCGGTACTATGCTTTGGCCTTGTG
CAGCCAATCTTGAGGACGTTAAATCCGGGGCATCCATTTGCTAAGGCATTAGTGATGGGCATCGCACTGGCGTCGAATGTTGG
GGGTATGACATCACCAATATCATCGCCACAGAACATCTTCGCTATAGAGCGGATGTCCATGGACGGCCACCCCCCTAGCTGGC
TTTCATGGTTTGCTGTCGCGCTGCCAGTGTCCATCATAATTAACCTGGTATGCTGGGCATTACTCCTGTTGGTCTACCAGCCA
GAGCGCTACATCACTGAAGTGCGGCGCGTCAAGCCAAATACGGACCCAGTAAACGGGACACAGGTGTACATTGTGATAGTGTC
ACTGCTGACCGTTGTCTGCTGGTGTGGCAACTCATATCTGCAAAGATACACCGGCGAAATGGGCATTATTGCTATTGTCCCAT
TGGTTGCGTTCTTTGGATTTGGCGTTCTCAATAAGGATGATTTCAACTCGTTTTTGTGGAATGTTGTTATGCTGGCCATGGGT
GGCTTGTCATTAGGTGAAGCCGTGAAGAGCAGTGGCCTTTTGGCAGCCCTCGCGTTAGACATCAGCAATCTGGTTACCGGGCT
CAGCTTATGGCAGGTCGCGGCCGTATTCTTTGGCATGGTTTTGGTGGCGACGACTTTCATCAGCCATACGGTTGGCGCTATGG
TCATTCTACCGATAGTGCAGTCTGTCGGCGAGGCCATGGCCAGCCCGACGCACCCGAAGCTGCTTGTCATGGGCGCGGCTCTC
ATGTGCTCAGGCGCCATGGGTCTTCCAGTAAGTGGCTTTCCGAACATGAACGCCGTTAGCCTCGAGGACAGCACTGGCAATGC
TATTGTTAGCACGAAAGACTTCCTCCTCGTGGGTGTGCCATCATCTTTCTTCGCATATCTGGTCATAGTTACGCTTGGGTACG
TCCTGATGCTCTTGGTAGGGTTA
>Planophila_terrestris.PTC1
ATGAAGTTTTCCCACTCCCTCAAGTTCAACAGCGTCGCAGAATGGAGGCAGCACTACATAAATTACGGCGCCTTGAAGAAGCT
GTCTTATGCGATCGAAAAGCAGGAGGAGGAAGGCCGGCAGCGCGAGATGTTTGCGCAGCAAAGCATGGACCTGCGGCAGCGCT
CGATGCGCCAGAGCGTCGATCTGCCGCGGGGCGCAGTGGAGGAGGACGAAGCCCGGCGGCGCGCGTCGCTCGACGCATCCTCC
TCGTTCACGGGCTCCGTTCAGCAGCCGCTCCTGCAGCGGCTCTCGTCTGGCCTGGGCGGGTCTTTGCGTTTGTCCATGCTGGA
GCGCAGTGCGGAAGAGGGCGGCAAGGTCTCGCAGGCCGACTTCCAGCGCAGCCTCGACTCGGAGCTGCATAAGATAGTGGACT
TCTACATCACAAAGGAGGCGGAGCTGAAGAAGGAGCTTGCGGCCGCGGAGCTCGATGCCCGCGCCGCCGAAGCGTCGTCTGCG
GGATCGTCTGGCTTTGCAGAGATGCAGGCGGAGCGGCTGCCGCGGCCGTCGTTCTGGCGGACCGCCGCGTCCGACGCGCTCAA
AGCCAAGATGCACGAGCGCCTGTGCTCGCTGTACGTGCAGCTGGTGGACCTGCTGAACTTCATCGAGCTCAACCGCACGGGCT
TCCGCAAGATCCTCAAGAAGCACGACAAGGTGACCGACGTGTCGCTCATGACCGACTACATGCCCGTCGTGACCAGCAAGCTG
AGCTCCAAGCGCGAGGAGGACCTTGGGGCGATGATCAATGAAGTCATCAAGCTGCACGCCATGGTAATGCACAACTCAGACAC
GAACGCTTCGGAGGTCGACCTCAAGCGCAACTTGCGGGATCACGTGACATATGAGCGCGACACCATCTGGCGCGACATGGTTG
CGCTGGAGCGCCGCAACATTACTGTCAAGGTGCCGGAGGGGGCGCCGACGGGTGGGTTTGCAAAATGGTGGCAGATATACCAC
ACGCCCGTCATGGTGGCGGCGGCGCTGCTGCTCTTTTTCGTCATCCTCAACATCGACATCTGGCCGAACGACACGCCCAAGCG
CCGCTGCGCCGCGATGCTCGCTCTCCTCGTCACGCTGTGGACCAGCGAGGCTTTGCCACTGTACGTCACGTCCATGCTGGTGC
CCCTGCTGACTGTGTTGCTGCGCATCCTCCCGGACGAAGCTGCCCCAGACGGACACCCTCAGCAGCTGCCGGCGCCGAAAGCC
GCGGAAGCCGTCTTCAAGGTGATGTTTTCGCAAGTGATCATGCTGCTGCTTGGTGGCTTTGCCATCGCAGGCGCTCTCTCAAA
GCACTACATCGCCAAGGCCATGGCGTCCAATATCTTGTCCCGCGTCGGCACTCGCCCACGGGACGTCATCCTGGCCAACATGT
TTGTTGCAACGTTTGCCAGCATGTGGATCTCCAACGTGGCGGCGCCGGTTCTGTGCTTCTCGCTCATACAGCCGATCCTGCGC
ACGCTGCCGAGTCACCATCCCGTCAGCAAGTGCCTGGTGATGGGCATCGCGCTGGCGTCCAATGTGGGCGGCATGACGTCGCC
GATCTCGAGCCCCCAGAACATTTTCGCAATTGAGCTGATGGCGCGCGACGGCGCAGCGCCCAACTGGCTCACGTGGTTCGTGG
TCAGCATCCCGGTGTCGATCGCGTCCAACCTCTTCATCTGGGCCATCCTGCTGGCGGTGTACCGGCCCGGGCTCGCGATCCGA
GAGGTCCGCCACATGCGCCGCGTGCAGGAGCCGATCACCAAAGTACAGGTGTATGTGGTCGCTGTCAGCAGCCTGACGGTCGC
GCTCTGGTGCGCCAGCTCAGCGCTGGAGCAGTACCTCGGCAGCCAGGGCATCATCGCGATCTTTCCGCTTGTCATGTTCTTCG
GCCTCGGCGTTCTCGACAAAGACGACTTCAACAACTTTCTCTGGCATGTTGTCATGCTCGCTCAGGGGGGCCTCGTGCTTGGG
CTGGCGGTGAAGGGGTCGGGCCTGCTGGCGGACGTGGCAGGCGCGATCCGCGGCGTCACGGCCGGCATGTCGCTCTTCGGCAT
CCTCTTCACGTTCTGCGCGCTGGTGCTCGTATGCACGACCTTCATCTCGCACACCGTCGGCGCGATGGTCATCCTGCCCATCG
TGCGCTCCGTGGGGCAGCACCTCTCCCCGACGCCGCACCCGCGGCTGCTCGTGATGGGCGCCGCGCTCGCGTGCTCCGGCGCG
ATGGGCCTGCCCGTGTCCGGATTTCCAAACATGAACGCGGTTTCGCTTGAAGATGCCACTGGAAAGACTTACGTGAACACACT
CGATTTTCTCATGGTGGGCCTGCTGGGAAGCTTGACTACGTTCTGTGTCATTGTGACGCTGGGCTACGCTCTGATGATG
>Pteromonas_angulosa.PTC1
ATGAAGTTCACGCACCAGCTCAAGTTCAACTCCGTGCCGGAGTGGCGCGACAACTACATTGACTATGCGCACCTCAAGAAGTT
CATCTTTGCCATCGCGCGGGCGGAGCAGGATGACATCCAGCAGCTGCATGGCGGTGCTGATGGCACATCCATGCCCTTGCTAC
AGCACACGGTCACCATGGGGCACGACAAGGTGGACGCCACGGAGGACAACCTCCGCCAGGCGCTGGACAAGGAGCTGCAGCGC
GTGATCAGCTTCTACATGACCAAGGAGGCGGAGACGCTGGCCAAGGTGACTTCCATGGAGCTTGAGATCAACACGCTGGAGAT
GACCCGTGCCCCCCGAGGCACCTCCATGGACCACATGCAGGGAGCCCAGCGTGGAGGATCTGGTGGATCCGGTGGCTCCGGAG
GCGGAGTGGACCTGAACCAGCAGCTGCCATCGCCTCCCCAGGGCCTTGCCACGGACGTGGAGGCCACCCCCCCAGCGGCGCAC
GTGAGCGCCAGCATGGCAGCCCCCAAGTCACCTGGCACCATGTCGCGCCAGATGCGTGTGGACTTCTGGGCGCGCGCCAACCC
CGGCGCCAGGCACGGCGGGTCGTTCGGCGGCGGCTCCGCGGCCTTCCTCTTCGTGCGCGAGCTGCAGTCCCACAAGGAGCGCC
TGCGTGTCGTGTTCAGCGACCTCTACCTGCAGCTGCACGACCTGCTCAACTTCCTGAGGGTGAACAAGGAGGGCTTCCGCAAG
ATCATCAAGAAGCACGACAAGATGACGAGCAGCAACCTGAAGGAGCACTACTGGCCAGTCCTGGAGTCCAAGTACCCCATCGT
GCGCGCCGACATGCTGGAGGCAACCATCAACTCGCTGGTCGACCTCTACGCAGTCATCTACAACCAGGGCAGCGTGGAGCTGG
CCAAGGACCACCTGGACAAGCTGCTGCGCGACCAGATCAAGGTGGAGCGCAACACGGTGTGGCGCGACATGGTGGCCCAGGAG
CGCCGCACCACCGCGGCCGTGGTGGAGGGTGCGGTCAGGCGGCCCTGGTGGGCCCAGCTGACCCCCCACATCGCACTGCTGAG
CAGCGTGCTGGTGTTTGCAGTCCTGCTGTCCATGGAGGACATCTTCGAGGGCGAGCCGGAGAAGCAGAACTGCCTGGCGCTCC
TCATCTTTGTCTCCATGCTCTGGGCCACCGAGGCGGTCCCACTCTACGTCACCTCCCTCGCCATCCCCCTGCTGGTGGTGGTG
CTCAAGGTGCTGATGGACAAGAGCGTGGACCCCCCCGTGCGCATGACGGCGCAGCAGGCCGCGCCCGCCATCTTCCACTCCAT
GATGTCGCAGGTCATCCTGCTGCTGCTGGGCGGCTCTGCCATCGCCTCCGCCCTCACCAAGCACTTCATCGCCAAGAAGCTGG
CCCAGGTGGTGCTCTCGCGCGCCGGCCGGCAACCACACAATGTGCTGCTGGCGCTGATGTTGGTGGCCGTTGTTGCCTCCATG
TTCATCTCCAACGTGGCTGCGCCTGTGCTGTGCTGGTCGCTTGTGGACCCGATCCTCAAGGCCTTTGACGCAGAGAACCCGTT
CTCCAAGTCGCTGGTCATGGGCATCGCGCTGGCCTCCAACATCGGCGGCATGACCTCCCCCATCTCCTCCCCCCAGAACATCT
TTGCCATCGAGCGCATGAGCATGGACGGCCACCCGCCTTCCTGGCTGGCGTGGTTCGCCGTCGCCCTGCCCGTCTCCTTCATC
TGCATCCTGGTGTGCTGGGGCCTGATCCTCGCCGTGTATCGCCCCTGGACCAAGGTGGCTGAGGTGAGGCCCCTGAAGCCAAG
CTCCGACAAGGTCACCTTCACGCAGTTCTACGTTGTGGCCGTCACCGCCGTCACCGTCACCCTGTGGTGCTTCAACACCCAGC
TGCAGCCGTACACCGGGGAGATGGGCGTGGTGGCCACCATCCCCATCATCGCCTTCTTTGGCTTTGGTGTGCTCAACAAGGAC
GACTTCCTGTCCTCCCCCTGGCTGGTCATGACGCTGGCCATGGGCGGCCTGGGCCTGGGTGAGGCGGTCAAGAGCAGCGGCCT
GCTGCTGTCCATCGCACACACCATCGGCGACGTGGTGCAGGGCATGGACGTCTTCACCGTCTGCTGCATCTTCTGCGCCCTGG
TCCTGGTCTGCACCTCCTTCATCAGCCACACGGTGGGCGCCATGATCATCCTGCCCATCGTGCAGTCGGTGGGCGAGCAGATG
CCCGGCCCCCACCACGCCAAGTTGCTGGTCATGTCCTCCGCCCTCATGTGCTCCGGAGCCATGGGCCTCCCTGTCAGCGGCTT
CCCCAACATGTTCCTCATCAGCAAGGACGACGGCACGGGCAAGAACTACATCAACACGCTCGACTTCATCAAGGTGGGCGTGC
CCGGCTCCATCGGCGCCTACTTTGTGATTGTCTCCGTCGGCTACCTCCTCATGCTGGCGGTC
>Asteromonas_gracilis-A.PTC1
ATGAAATTTTCGCACCAACTGAAATTCAATAGCGTAGCTGACTGGAAAGAGCATTATATTCATTATGCTAACCTTAAGAAAAT
CATTTACGAAATCGCTCGTCTGGAGCAGGCGCGAGCCAATCCAGATGCCGGAGAGGTCACAGAATTGGGGGAGCCCTTACTTT
CCAGACCACCAGTTCAAAATTACGAACTTGCTATTTCTACAAAGGAATCTGAATTTGTCGGGGAGCTTGATCGTGAGCTGGCT
CATATCATTACATTCATCTTGCGGAAGGAGGCTGAGTTGGTGAGCCAGCTTGAAGCACTAGATCTCGAGGTCCACAGCTTGGA
GAGTGCAGATCCCCAATATCGTAAGAGCTTGGATAGGGATTTTTTGGACCAGGATGCTGCAGTAGGAGCTGAGAATGGGACCG
GGTATCAAGCGGGCATTCCAGCTCGACCTGAGCGAATCAAGTTCTGGTCCCAAGGTGCTGAACCTCACCTTGCTGCTCGTGAT
GCGCGAAATGTGGCCCAACTGAAACCTGCACAAAGAGAGGCTCTGTCACAGAAATTCGTCGACCTTTTTACCACTTTAAATGA
CCTATTGGAATACCTGGTCCTCAACCGCGAAGGCTTCAGGAAGCTGATCAAGAAGCATGATAAGATGACGTCTTCAGCAAGCA
TGAAGGAAAGCTACTGGCCTCTGATTGAGCAGCGTTATCCAGAGCATAAGAGGGTTTCAATGGGACAGAATATTGAGCGGCTG
GTGGACCTGTATGCTATTCTGTTCGAGGGGGGTGACACGTCGTCAGCCAGAGAGGCCCTGTCACAGAATCTGCGGCAGCATAT
CAAAGTAGAGCGAAACACTGTCTGGAGGGACATGGTGGCAATGGAGAGGCGCACAGTTGCTGCAACAGTAGACGCGCCTAAGA
GGAAGAGAGCCTGGTTCAGCACTCACAGGAAGCACCTGTCCCTCCTGCTGGCTTCGATTGTGTTCGCATCTATGCTCAGCTTG
AAGCTTTTTAAAGAGCCTGAGAAATCGAATTGTGCGGCGATTCTGGTGTTCGTGTCGCTTCTCTGGGCGAGTGAAGCTATCCC
CTTATTCGTGACCGCAATGGTGGTCCCTGTCCTGGTGGTGTCGCTGCGTGTTCTTGTTGACGACTCTAGCGCAAAGCATCCCA
TCCGCCTTTCTTCCACGGATGCTGCCAACGCCATCTTCCACGCCATGTTCTCCCAGGTTACCATGCTGCTCCTGGGTGGCTTC
ACCATCGCAGCCGCCTTGTCCAAGCACTTCATTGCCAAACAAATGGCGGTTGCAGTGCTGTCCAGGGTGGGCAGGCTTCCGCG
CAACGTCCTTTTGGCGTCGATGTGTGTCGCTGCCTTCGCATCCATGTGGATCTCCAACGTTGCCGCACCGGTGCTGTGCTTCT
CTCTAGTGCAGCCCATTCTGCGCACACTGGACGTGAGCTCACCCTTCGCGAAAAGCCTCGTCATGGGCATTGCCCTCGCCTCA
AACATAGGGGGTATGACCAGCCCTATCTCCTCCCCCCAAAATATCTTCGCCATCGAGCGCATGTCCATGGATGGCGTCGCTCC
CAGCTGGCTGTCCTGGTTCGCGGTGGCGCTACCCGTCTCTTTCATCAGCATTATCCTCTGCTGGCTCCTCCTCCTCCTCGTGT
ATCGGCCTGGGATGTCTACAACCGAGGTCCGGCCTCTGAAGCCGTATACGGACCCTATGAACATGACGCAGGTGTACGTGATT
GTGGTGTCCATGGTCACGGTTTTGCTGTGGTGCGCCAACTCCGAGGTGCAGCAGTTTGTGGGGAACATGGGGGTTGTGGCGGT
GCTGCCCATGATTGCTTTCTTCGGCTTTGGGGTGTTGAGCAAGGACGACTTCAATGGCTTCCTATGGAACGTGGTCATGCTGG
CCATGGGGGGCTCGGCCCTGGGGGAGGCTGTGAAGAGCAGCGGCCTGCTGTCCACGTTTGCAAATGACATCAGCGGGCAGGTA
CATGGGCTTGACTTGTGGACCGTCAGTGCGATTTTCTGCGGTGTCGTGCTCATCTGCGCCACGTTCATCAGCCACACGGTGGC
GGCAATGGTCATTCTCCCCATTGTCCAGTCCGTCGGCGAGGCGATGCAGGAGAACCCTCATCCAAAGCTGCTGGTCATGGCCA
CTGCCCTCACGTGCTCAGCTGCCATGGGCTTGCCCGTGAGTGGCTTCCCCAATATGAATGCTGTGTCACTGGAGGATGGCACC
GGCCAAACCTTCGTCAACACCCTGGACTTTTTGAAGGTGGGCGTCCCCAGCTCCGTCGCTACGTATTTCGTGATTATTTCTGC
GGGCTACTACTTCATG
>Haematococcus_pluvialis-B.PTC1
CGGGTGCGGTTCTGGGCAGAGCTGGGCATGCGGCGCGGCGGCAGAGACTTGCGGTTTGCCCGGGATGTCATGCGCATCCGCTT
CCATGACTTGTACACCAGCCTGAACGACCTCATAGAGTACCTCAGCTTGAATAGGGAGGGGTTCCGCAAGCTCATCAAGAAGC
ATGACAAGCTGACCAGCACATGCCTCAAGGAGGCCTACTGGCCTGACTTTGAGCGTCGCTACCCCATGAAGCGCAAGGAAGAG
CTGGAGCGGCACCTTGATCGCCTCATAGAGCTGTACGCTGTCATGTTTGCTGGCGGAGACACGCGCAAGGCCAGGGACCTGCT
GCTGAAGACGCTGCGAGAGCACATCAAGGTGGAGCGCAACACAGTGTGGAGGGACATGGTGGCACTGGAGCGGCGCACTGTGG
CAGCCACTGTGGGCGCTGCTTCTGGGGTAGCTCGCCTCAGCAAGTACAAGGCATACAGCGAGCGCCTTGGCCTGCTGGCAGCC
CTCCTGGTCTTTGCTGCGCTGTTGTGGGCACCAGTGTTTGAGGAGAAGGAGAAGAGCAACTGCCTGGCCATTCTCGTCCTGGC
ATCCATGCTTTGGGCCACAGAGGCCATTCCTTTGTTTGCCACGGCCATGCTCATCCCCGTACTGGTGGTCATGCTCAGGGTGT
TGGTGGACCACGGGCGGCCTGCAGGTGCCCAGCGCCTCACCCCTCAGGAGGCGGCCCCCCTCATCTTCCACGCCATGTTCTCC
CAGGTCATCATGCTGCTGCTCGGGGGGTTCACCATAGCTGCTGCCCTTTCAAAACACTTCATCGCCAAGCAGATGGCAGTAGC
AGTACTGTCCAGGGTGGGGCGTAAGCCACACAACGTGCTGCTGGCCTCCATGTTTGTGGCCATCTTTGCCTCCATGTGGATCT
CTAACGTTGCTGCGCCTGTGCTGTGCTTCTCCCTGGTGCAGCCCATACTCAGAACCTTGGATGTAAACACTCCCTTTGCCAAA
TCCCTGGTCATGGGGATAGCCCTTGCCTCAAACATCGGTGGTATGACCTCTCCTATCTCCTCACCCCAGAACATCTTTGCCAT
TGAGCGCATGTCCATGGATGGCAACCCACCCAGCTGGCTCAGCTGGTTCTTCGTGGCACTGCCTGTGGCAATCATCAGCAACT
TCATCTGTTGGGCCGCCATTCTCCTGGTGTACCAGCCCTGGCACAAGACGTCTGAGGTCCGGCCCATCAAGCCCAGCTCGGAC
CCTGTCACCTGGACGCAGGTGTATGTCATATTTGTGTCGCTGGCGACGGTGGGTTTGTGGTGCGGCAACGTGGCCCTGCAGAA
GTACACAGGGGAGATGGGTGTGGTGGCGGTGCTGCCCATGGTGGCATTCTTTGGGTTCGGTGTGCTCAACAAGGATGACTTCA
ACGGCTTCTTGTGGAACGTGGTGATGCTGGCCATGGGCGGTTCAGCCCTGGGGGAAGCTGTGAAGAGCAGTGGCCTGCTGTTG
ACCATTGCTCAAGGCATCCAAGAGATGGTGGATGGCCTGAACCTGTGGACTGTCACCATCATCTTCTGCGCCTGTGTGCTGGT
GTGCACCACCTTCATCTCTCACACAGTGGGCGCCATGGTCATCCTGCCTATCGTGCAAAGCGTGGGGGAGAGCATGCCCGGCC
AGCCCCACCCCAAGCTGCTGGTCATGAGCGCAGCACTCATGTGCTCAGGCGCCATGGGCTTGCCCGTCAGCGGCTTCCCCAAC
ATGAATGCCGTGTCCCTGGAAGACTCCACAGGGCAGACATATGTCGGAGCACTCGACTTCATCAAGGTCGGGGTACCTAGCTC
GATCCTAGCGTACGCCGTGATCATCACGGTCGGGTATTCGTTGATGTTGATCATTGGCTTC
>Chlamydomonas_bilatus-B.PTC1
CAGACCATCATGCTCCTGCTCGGTGGCTTTGCCATCGCCGGCGCGCTGAGCAAGCACTTCATCGCCAAGCAGCTCGCGATCGC
CGTGCTGTCGCGCGTCGGGCGCAAGCCCCACAACGTGCTGCTCGCCGCCATGTTTGTGGCGACGTTTGCGTCAATGTGGATCA
GCAACGTGGCAGCACCGGTGCTCTGCTTCTCCATCATCATGCCCATCCTCAAGACGTTGGACACAGCCAGCTCGTTCGCCAAG
TCCATGGTGATGGGCATCGCGCTCGCGTCCAACGTGGGCGGCATGACATCGCCCATCAGCAGCCCGCAGAACATCTTTGCCAT
CGAGCGCATGTCCATGGACGGGCAGCCCCCCTCCTGGCTCGCCTGGTTTGCGGTCGCACTGCCCGTCGCAACGCTCTGCAACC
TGCTGTGCTGGCTGCTCATCCTGGCCGTGTACCAGCCCTGGCGCACCATCAATGACGTGAGGCCGCTCAAGCCCAACACAGAC
CCCATGAACTTCACACAGGCTTACGTCATCTTCATCTCATTGGCGACAGTGGGGCTGTGGTGCGCCAACACCAGCTTGCAACA
GTACACTGGCGAGATGGGCGTTGTGGCTGTACTGCCCCTGGTTGCCTTCTTTGGCTCCGGAGTGCTCAGCAAGGATGACTTCA
ACGGCTTCCTCTGGAACGTGGTCATGCTGGCCATGGGAGGCCTGGCGCTTGGCGAGGCTGTAAAGAGCAGCGGCCTGCTCCAG
TCCATGGCAGAGGGCATCACGGAGGTGACGGATGGGATGGACCTCTACCAGGTGCTGCTGGTCTTCTGCCTCATGGTGCTCAT
CAGCACCACCTTCATCAGCCACACGGTCGGCGCGATGGTGATCCTGCCCATTGTGCAGAGCGTGGGTGAGGCTATGCCGGGCA
GCCCGCACCCCAAGCTCCTGGTCATGGCCAGTGCTCTCATGTGCTCAGGCGCCATGGGCCTGCCAGTCAGCGGCTTCCCCAAC
ATGAACGCCGTTTCACTCGAGGACTCAACCGGGCAGAACTACGTGGACACGCTGGACTTCCTCAAGGTTGGAGTGCCAGGCTC
GGTACTCGCCTATGGCGTCATTGTCACCCTGGGCTACAACCTCATGCTCATGGTGCGCTTC
>Vitreochlamys_sp.PTC1
ATGAAGTTTAGCTCTCTTTTAAAGTTCAACTGTGTACCTGAGTGGCGTGACCATTACGTTCAGTATGGCCACCTGAAAAAGTA
CATATACGCGTTGGCGAAATGGGAAGCGGACCACCTACACGAAACTCAGCCTCCGGACCTGGAGTCGCTCACGTCTCCCTTGC
TGCCAACCAGTGGACTGGGGTCTGCTTATGGACCCAGCGAGGAGGCCTTCCAGCGTGAGCTGGACCAGTCACTGCTGGAGGTG
ATCCGCTTCTTCAGCATGAAGGAGGCAGAGTTGGTGTCCAAGTGCCAGGCGCTGCTGCTGGAGCTGGTCTCGGTGGAGAAGCT
GCCGTCCGGGTCGTCCGCGGGGCGCCGCTCCTTCAGCGGCGCCTCCACGCCCTCGGGTGCCGCCACGCCCACGTCCTCGGCGC
CCCACGGCTCCACCGCCAACGTCCTGGCCGGCGCCAAGTCGCGTCTCACCGCCTCGCCGCAGACGTCGCCCCACGTGACGCTC
AGCGGCGCCAAGGGCGCGGGGGGGCTGGGCGGCATGCACTTGTCGCCCTCCGTCGTGCATCTCATGGACGTGGCTAACCACAA
GGACCACCGTACAGTGCGTGTGGAATTCTGGCGCAAGCCCCCGCGCCGCCTGTTCCAGAACCTGGAGGCCGCTCGCAGCAAGC
TCAAGCCCAGGCTCCAGGAGCTGTACATTGCTCTGCACGACCTTGCCGAGTTCCTCCACCTCAACCGCGAGGGCCTGCGCAAG
GTCGTGAAGAAACACGACAAGCTGACCCGCCGGGTCACGCTCAAGACCAAGTGGTGGCCCCAAGTGGAGCACCTCATCCCGCC
CACCAAGAAGGAGGAGGTGGACAGGGCAGTTTCGGAGCTGGTGGACAACTACGCGGTCTTGTTCACGGGCGGCTCAATGGCGG
CTGCTGAGCAGGCGCTCAGCCAGGGGCTGCGTGACTACGTGACCATGGAGCGCAACACCGTGTGGCGTGACATGGCTGCCATG
GAGCGCCGCTTTGCATCCCTCGCAGTCAAGAAGGGCTCCGCAAGCTTCATTGCCACCTGGTGGACGCAGCCGCTCAAGATCGC
CGTGAGTCTGTTGGTGCTGTCGGTGCTGCTCAACGTGACGATCTGGCCCGAGGACGAGAAGAACAACTGTCTGGCGGTGCTGC
TGTTTGCGTCCATGATGTGGTCGCTGGAGGCTATCCCGCTGTTTGTCACGTCCATGACCATCCCCTTCCTCGTCGTCTGCTTC
CAGCTGCTGGTGGACCATTCGCAAGACCCACCCGTGCGTATGACCGCGCAGCAGGCTGCACCCGCCATCTTCCACGCCATGTT
CAGCCAGGTCATCATGCTGCTGCTGGGGGGCTTTGCCATCGCAGCGGCGCTTTCCAAGCATGCCATCGCCAAGCAGATCTCAG
TGGCTGTCCTCTCACGCGTCGGGCGTAAGCCGCGCAACGTGCTCCTCGCTAGCATGTTTGTGGCTACATTTGCGTCCATGTGG
ATTTCAAACGTGGCTGCGCCCGTGCTGTGCTTTGGCCTCATCCAGCCCATCCTCCGCACCCTCGACCCAGGCCACCCTTTCGC
CAAGTGCTTGGTGATGGGCATCGCGCTGGCCAGCAACGTGGGCGGCATGACGTCGCCCATCAGCTCCCCGCAGAACATCTTTG
CCATTGAGCGCATGTCCGTGGACGGCAAGGCGCCTTCGTGGCTGTCGTGGTTTGCCGTCGCGCTCCCTGTGTCCATTGTGTCC
AACCTGATCTGCTGGGCGGTGCTGCTGCTGGTGTACCGCCCCTGGACCAAAATCCAGGAGGTACGACCCATCAAGCCCATCAC
CGACCCCATCAACGGCACGCAGGTGTACATCATCGTGGTGTCGGTGGTGACGGTGGCGCTGTGGTGCAGCAACACCATCCTAC
AACCGTACACGGGCGAGATGGGC
>Botryococcus_terribilis.PTC1
ATGAAGTTTTCCCAGCAGATCATATTCAATTCCGTTCCTGAATGGAAAGATAACTACATCAGCTATGCCCAACTCAAAAGGTT
GATATACTCCGAGGAAGCTGCACGTTTAGCGGCTGGCAGAGATGGAGCCCGGGGGGCATCGATGCGGCTGCAGCGTCTGCGGA
AGACAGCGATGCAGTTCAAGGACGATCTCAAGAAGGAAGCTGACAAGGTCGTCAGGTTCTTCCATGAGGAGGTGGAGAGTATC
TGGAGCAGGTTCCATCTGGTACTGCACGAGATCGAATGCTTTGAGCAGCAGGAGTGGCTTCCCCCCTCTGCTGCGGGCCTGGA
CACCTCCCCGACCAGCCCCCTTCTCTCCGCCACATCCATGCCCGCCACCCCCAAAACGCCCCCCATGCCAAGCCCGCGCTCCA
GCCCCTTCCAGAGGGCCGGCTCAGCCGGCATGGGGCTCTTGCGGACGGTGACGGGGATTCTCCCGCGGCCGAAGCGCAGCCCT
AGAACTTTGTCTGGGCCCTTGCTTGAGGTGGAGGATGGGACCCCCCGGGATGATTCTAAGACCTGGATTTGGCAGCAGGCAGA
ACCCAGTATTGCCCGGAAACGGGACGAGCTCAGGGGTCAGTTGTCGGAGGTGTACCAGGATGCAAACAACATGATCGAGTTCC
GGAGATTGAACCTGGACGGCTTTCGCAAGATCCTGAAGAAGTACGACAAAGTCCTGGAAGGCCTCCCCGGAGCAGAGAAACTG
TCTGAATCACAGTTTCCTGGGATCAAGGAGAGACTCGAGGCACTGGACTTGACACGGATGCAGGAAGTGGAAGGGGAGGTGGT
GAGGTTGTACGCGCGCGTGTGCTGCTCCGGTGTATATGCAGTTGCTGAGGAACTGCTCAAGAAGCAGAAGAAGGATCGCATCG
TCTTTGACCGGAGCACCGTCTGGAAGGAGATGGTCGAGCGGGAAAGAAAACGCAGCGCTGCGCACGTGGAGGGCGGGGCCGCG
CCTCGTGCCTGGTACCAGCGCCACTGGCAGCTCATGGCCTGCGCCTTCTGCGGGGCCGTGTTCCTCGCACTGCTCTGGGTGCC
CATCTTTGAGGAGGTGGAGAAGCAACACTGCGCCGCCCTCTTGGCCTTCGTCTCACTCCTCTGGTGCACGGAGGCCCTCCCGC
TGTTTGCCACTGCCATGCTGGTGCCCTTCCTGGTGGTGACCCTCGGGGTGTTGGTGGACCGCAGCGTTGACCCTCCCCACCGC
CTCACGCCCCAGGAGGCTGCCCCCGCCGTCTTCAAGACCATGATGTCCCAGGTGATCATGCTGCTGCTCGCGAGCTTTGCCAT
TGCCGGAGCGCTGAGCAAGCACTTCATCGCCAAGTGGCTGGCGTCGGTCTTCCTGTCGCGCTTCGGCAAGCGGCCCTCTCGGG
TGCTTCTGGCCAACATGGGGGTGGCCACGTTTGCCAGCATGTGGATTAGTAATGTGGCTGCGCCTGTGCTCTGCTTCTCCCTG
CTGCAGCCTATCCTCCGCAACCTCTCTGCAAAGGACAGCTTTGCAAAGGCACTCGTGCTAGGCATTGCCCTAGCAAGCAATCT
GGGGGGCATGACGAGTCCGATTGCCAGCCCCCAGAACCTGTTTGCAATCCAGCAGATGTCGGTCGGGGGCAATGCCCCCTCCT
GGCTCCAGTGGTGGTTGGTTGCTCTGCCTGTTGCCATTATAGGCAACCTGGTGTGCTGGGGCCTGCTGCTGTGGAGATACCAG
CCGCCCCCAGATGACGTGCGCGAGCTGCATGAAGCCAAGGGGTTCCACATCAACCCGACCCAGATGTATGTGGTGGCTGTCTC
ACTCCTGACCGTGGGGCTCTGGTGCTGCAACGGCTACTTGACTCCCTACTTTGGGGAGATGGGCGTCATCGCCATCATCCCGC
TGGTTGCGTTCTTCGGCACGGGTGTCCTGGACAAGGATGACTTCAACGCATTCCTGTGGAACGTTGTGATTCTGGCAATGGGC
GGGATGGCGCTGGGATCTGCAGTGGACAGCTCAGGTCTTCTGCTCACAATCGCAAAAAAGCTGGAAGGTCTGGTGTCCTCGCA
CGGCCCCTGGGTGGTACTGGCCATTTTCTGCGCGCTGGTACTGTTTGCGACGACGTTTGTCTCCCACACGGTCGGGGCCATCG
TCATCCTCCCGATCGTGCGAGCCGTCGGGGAGACCATGACGGACCCACACCCAAAGATGCTGGTCATGGGGGCAGCCCTCATG
TGCTCCGGTGCCATGGGCCTGCCCGTCTCCGGCTTCCCCAACATGAACGCAATCTCCCTTGAGGACAAGACAGGCGTCAACTA
CCTGACGACCAAAGACTTCCTGCTGGTGGGGGTCCCGTCGTCCGTCGCCACGTGGGGCATCATTGTCAGTGTGGGCTACGTGC
TCATG
>Eudorina_elegans.PTC1
ATGAAGTTTACACATACCCTAAAGTTTAACGCAGCCGACTCGTGGAGGGAACACTACATTCAATATGCACACCTGAAGAAGTA
CATTTACGCCCTTGCAAAAAGGGAGGCGGACCTCCAGGCTGGTGGCCATGTGCCCGACGACGAGTCTCTGCACGCGCCGCTTG
TACCGGAGACCTCTCGCTCTGGACAGGGCGTCAGCGAAGAAGGCTTTCAGCGGGAACTCGACGCCCAGCTGGCCGCCATCCTT
TCATTTTTTGCTGTCAAAGAAGCGGAACTGCTTGCGAAGGTGTCGGAGTTGGAGTTGGACTTGCAAAGTTTGGAGAAGATACC
TAATCGGCAAGAAGCGTCGACCATGTCGCGCCTCGGTGGCGGCGGTGGGGCAGCGGGCAGCAACCCAACCGGCAGCCCTGGAA
CTGCCGCCGTGGCGGCTGTCTCTGCGGTGTTGCCCTCCTTGTCGATTCTCTCTGTCAGTCCCTCCACCCTCGACCTGGCGCGC
ATGGTGAACTCCACTCCGCCGGAGGAGCACCGCAAGGTCCGGGTGAAGTTCTGGGAGAACCCGCCAAGGCACGTGTTCTTACC
GAGCCTGCATGCACGACGGACAAAGCTCCAGGGCCGCTTTCAGGATCTGTACATCGGCTTGCATGACCTGCGGGAGTACTTGC
ACATCAACAAGGAAGGCTTTCGCAAAATTATTAAGAAGCATGACAAGTTGACGCGTGCAGTGGATCTTCGCGTGCGCTGGTGG
CCCAACGTTGAGGCGCACCTGGCACCTGATGCCAAGCAGCAGGAGCTGGATCGGGCCATTGCAGCGCTGACGGATCACTATGC
GGTGCTGTACATGGGGGGCGACGTGGCTAAGGCTGATGAGCAGCTGTCCCACGGCCTCCGTGAGCATATTACGGTGGAGCGGA
ACACGGTGTGGCGCGATATGGCGGCCATGGAGCGCAAGTACGCAGCGGTGTCCGTCAAGCAGGCAGCGGCTCCTGGGGGCCTC
AGAGGCAGCTACCGCAAGCTCGCGGCCTGCTGTGCAGTGTTCGCAGTGATGTTGCACGTGAAGGTTTGGGGGGAGGATGAGGA
CGAGCCCAAGAACAACTGCTTGGCGTTGCTGGCGTTTGCGTCTCTGCTGTGGTCGCTTGAGGCCGTCCCGCTGTTCGTAACCA
GCATGGCCCTTCCGCTGCTCATTGTGGTAACGGGCGTACTGGTCGGGCCCGACAAGCAGCCCCTCACCCCGCAGCAAGCGGCC
CCGGCCATCTTCCATGCTATGTTTTCCCAGACGATTATGCTGCTGCTGGGCGGCTTCGCAATTGCGGCTGCACTGTCCAAGCA
CGCCATTGCGAAGCAGGTGGCGGTTGCTATTTTGTCCCGCGTGGGGCGCAAGCCCCGCAACGTGCTTCTTGCTGCGATGTTCA
CCGCAACCTTTGCGTCGATGTGGATTTCCAATGTCGCGGCACCAGTGCTGTGCTTCGGGCTCATCCAGCCAATCTTGCGCACC
CTTGACCCGGGTCATCCATTTGCCAAGTCTCTGGTGATGGGGATTGCGTTGGCGTCGAACGTTGGAGGCATGACATCGCCCAT
ATCATCGCCGCAGAACATTTTCGCGATCGAGTGCATGTCATTCGACGGCCATCCCCCCAGCTGGCTTTCCTGGTTTGCCGTTG
CCCTGCCGGTGTCCATAACATGTAACTTTGCCTGCTGGGCTGTGCTCCTACTCGTTTACCAGCCTGGGCGGGCAATCGCGGAG
GTGCGACCTATCAAGCCAAACACGGATCCCATCAACGGGACTCAGGTTTACATCATCGTCGTGTCGCTGCTGACCGTGGCTGC
CTGGTGCGCAAACACTTTCCTTCAAAGGTACACTGGTGAGATGGGTGTCATTGCGATTTTGCCGCTCGTTGCGTTCTTTGGCT
TCGATGTACTCAACAAGGACGACTTCAACTCATTCCTGTGGAACGTGGTCATGCTGGCCATGGGAGGGTTGTCGCTAGGTGAG
GCGGTCAAGAGCAGCGGCTTGCTGGCAGCGCTCGCATCGGACATCAGCGGGGTGGTGAAGGATCTCACCCTGTTCCAGGTGGC
GGTTATATTCTGCGGAATGGTGTTGGTGGCGACAACATTCATCAGTCACACAGTGGGGGCTATGGTCATCCTGCCCATCGTAC
AAACTGTCGGAAAGGCCATGGAGGGGACGCCGCATCCAAAGCTTCTTGTTATGGCAGCGGCGCTGATGTGCTCAGGTGCCATG
GGTTTGCCAGTGAGTGGCTTCCCTAATATGAACGCGGTTAGCCTGGAGGACAGCACTGGCAATGCCATCGTCAGTACGCAGGA
CTTCTTGTATGTCGGTGTGCCTTCGTCAATAATCGCCTACGGTATTATAGTTACCCTAGGATATGTGCTGATGCTGCTGGTTG
GGCTT
>Pandorina_morum.PTC1
ATGAAGTTTACACACCAGCTGAAGTTCAACAGTGTCCCCGAATGGCGGGAGCACTACATTCAGTACGCACATCTTAAGAAATA
TATTTATGCATTGGCCAAACGGGAGGCAGATCTGCAGGCTGGAGGCGACGAAGATGGACTCTTATCACCCTTGGTACCGGAGA
CGTCTCGGGCCGGTCAGGGTGTCAGCGAGGAAGGCTTTCAGCGGGAGCTTGATGCTCAGCTTGCGTCCATTCTTTCGTTCTTT
GCCGTCAAAGAAGCGGAGCTACTTGCGAAAGTGTCGGAGCTGGAATTGGATGTACAAAGCCTGGAAAAGATACCGAGCCGTCA
GGAGGCGTCTGTCTCCCTATCACGCTTGGGCGCTGGCGGGGGATCAGGGGGAGGGAACCCCACGAGCAGCCCCGGGTCGGCGG
CCGTGTCGGCCGTGTCCGCCGTGCTGCCCTCGCTGTCGCTGCTATCGGTCAGCCCCTCCACACTCGACCTCGCACGGATGGTC
AGCTCCACCCCGCCCGAGGAGCACCGGAAGGTGCGGGTCAAGTTCTGGGAGAACCCGCCGCGGCACGTCTTCTCACCAAACCT
ACACGCGCGGCGGGCGAAGCTGCAGGGTCGTTTCCAGGACCTGTACATCGGCTTGCACGACCTGCGCGAGTACCTGCACATCA
ACAAGGAGGGTTTCCGCAAGATCATCAAGAAGCACGACAAGTTGACGCGCGCGGTAGACCTCCGAGCACGATGGTGGCCCAAC
GTGGAGGCGCACCTCGCGCCCGACGCGAAGCAGCAGGAGCTCGACCGCGCCATCGCGGCGCTGACGGACCACTACGCGGTGCT
GTACACGGGTGGCGACGTTGAGAAGGCTGAGGAGCAGCTGTCGCGCGGCCTGCGGGAGCACATCACGGTGGAGCGCAACACAG
TGTGGCGCGATATGGCGGCCATGGAGCGCAAGTACGCGGCCGTGTCGGTGAAGCAGGCGGCGGCGCCCGGCTTACTGCGGTTC
AGCGCCAATCGGGCGCATGTGCGATGGGCAAAGCTGGCGCTCTGCTGCGTGGTGTTCGCCATCCTGCTCAACGTGGACTTTTA
CAAGGAGAACGATATGGAGCCGCCCGACGTACAACGGGCAAAGAATAAGTGCCTCGCCTTGCTGGTGTTCGCATCCATGCTGT
GGTCTCTGGAGGCGGTGCCGCTATTCGTGACTAGCATGGCTCTGCCGTTTCTGATCGTCATGCTGGGGGTCCTTATGGACTCC
GACGGCAAGGAGCGGCTCCAGCCCAAGAGTGCGGCGCCCGCCATTTTCCACGCGATGTTCTCCCAGACGGTCATGCTTCTGCT
CGGCGGCTTCGCCATTGCGGCTGCTCTGTCCAAGCATGCAATTGCAAAGCAGGTGGCGGCGGCCATCTTGTCGCGTGTGGGAC
GGAAGCCCCGCAACGTGCTGCTCGCCGCCATGTTCACCGCGACGTTCGCGTCAATGTGGATCTCCAACGTCGCTGCACCGGTC
TTGTGCTTCGGGCTCATCCAGCCAATCTTGCGGACACTCGACCCTGGCCACCCCTTTGCCAAGTCACTCGTGATGGGGATCGC
ACTGGCATCGAACGTCGGCGGCATGACATCTCCAATCTCGTCGCCACAGAACATTTTCGCCATCGAGGAGATGTCCAAGGGTG
CCAATCCGCCGAGCTGGCTTTCCTGGTTCGCCGTGGCGTTGCCGGTTTCCATCGCATGCAATCTGATTTGCTGGGCCGTGCTG
CTCCTGGCGTACCGGCCTGGCCACGTCATCTCCGAGGTGCGGCCCATCAAACCCAACACGGACCCCATCAATGGCACACAGGT
GTACATCATCGTGGTGTCGCTCTTGACCGTGGCTGCCTGGTGTTCAAATACATTTCTACAAAGGTACATCGGTGAAATGGGCG
TCATCGCCATCGTGCCACTGGTGGCGTTCTTCGGGTTCGACGTGCTCAACAAAGACGACTTCAATTCGTTCCTGTGGAATGTC
GTCATGCTCGCCATGGGAGGCATGTCGCTCGGCGAGGCTGTCAAGAGCAGCGGGCTGCTGTCGGCGCTTGCGACGGACATCAG
CAACTTGGTACTTCACTTATCCATCTTCCAAATAACTGTCATTTTCTGCGGCATGGTGCTGGTGGCGACCACCTTCATCAGCC
ACACCGTCGGAGCCATGGTCATACTGCCGATTGTTTGGTCCGTGGGCGAAAAGATCAAGGGTGACGACCCAGCCAGTCAGAGC
CATTCAAAGCTCCTGGTGATGGCTGCGGCGCTCATGTGTTCAGGTGCCATGGGTTTGCCCGTAAGCGGCTTTCCAAACATGAA
CGCTGTGAGCCTCGAAGACAGCACAGGCAATCCGATCGTAAACACGCAAGACTTCATCTACGTTGGTGTGCCTTCGTCAATTT
TTGCATACGGGGTCATCGTCACCTTGGGTTACGTGCTGATGTCGTTGGTGGGCTTT
>Oedogonium_foveolatum.PTC1
GTGAAGTTCACACACTATCTTAAGTTCAACAGTGTTCCAGAATGGAGAGGCCAATACCTGGCGTATGGCTTATTGAAGAAGCT
TATATACAAGCAGGAGAAGCTGCTTGCTTTAAGCCGGGCGGCTCCCCATCCAGAATCCGTTGACATTGAACATGAGGAGCCCA
CGGTGGAAACGCCATTCTTGCAAGTCCCGTCGACACCACCATCTCAGCTGGATTTGTCCCCCCGTCGCTCCTTTGACCGCAGC
TTCCTCTCGGGTAAACTGTCCCCCCGCAGCGCGAGCACAACGGGCAACCCTGAGATTGAGTTTGTGAGGTTATTGGGCTCGGA
GCGCACTCGCCTCAATGAGTTCATCGCGAGCAAATATACGGAGCTGACTGGGCAACTATCCAACGTGACAGAGGTGATGCGAG
TGAAGGAGATGGAGGGCGGCCTGCCCCACTCGGACCCCAACCCATACAGCCTGGCGGCACATCGGGTGGCGTTCTGGAGCCAG
GCTCCCATGCAGAAGGCGCGCGAGCAGCTCATCCCGCAGCTGGTGGAGCTGTGCGTGTTCCTGACGGGGCTCAAGGACTACGT
GGAGATGAACAAGGAGGGGTTCCGGAAGATCCTGAAGAAGTGGGACAAGGTCAACGAGGCGCGGCTGAGCGAACAGGAAATGC
CGCTGGTGGAGCAGACGCTGGACGTGGGGCGGAGGCTGCAGGACCTTGATGAAGCCATTGGTCACGTGATGTCTTTGTACGCG
CTGCTCACCTCCAAGGGCAACATGGACCTGGCCTGGCGCAGCATGAAGGAGCACCAGTCGGAGCACATCAGGTTCCAGCGCAG
CACCGTGTGGCACGACCTGATCGCCCTCGAGCGCCGCACCCTGACCGCCACCGCCGTGCGGCCTGTAGATGAGGTGATGGGCT
GGTGGGCTGTCAACCGGAAGCACTTCATGATTGTGGCCTCGCTGATGGTGTTCCTGCTGCTGCTGGAGGCCAAGACGTTTGAG
GGTGACGAAGCCGCCCCGCAGCGGAACTGCCTCGCGCTGCTGGTCTTTGTCTCGTGCCTGTGGGCTACAGAGGCGATCCCGCT
GTTTGTGACGAGCATGCTGGTGCCGCTGCTGGCGGTGTCGCTGCGTGTGGTGGTGGTGGACGGGAAGCGGCTGGAGCCACCAG
ATGCGGCCACCTTCATGTTCGGGAAAATGTTCTCACAGGTGATCATGCTGCTGTTGGGCGGCTTTGCCATTGCAGCGGCCCTG
AGCAAGCACAACATTGCGCGCAAGATGGCCATATCGGTGCTGTCCCGCGTGGGGAGAGCCCCGGGGGGGGTCTTGTTGGCGAC
CATGATGGTGGCCACATTCCTGTCGATGTGGATCTCCAACGTGGCCGCGCCAGTGCTGTGCTTCTCTCTCGTGCAGCCCATAC
TGCGGACTCTCGACACAAACCACCAGTTTGCCAAGGCGCTTGTGATGGGCATCGCGCTGGCCTCCAACGTGGGCGGCATGACC
AGCCCCATCAGCAGCCCGCAGAACATCTTCGCCATCCAGGTCATGTCAGGCGGCGGCCACAGCCCCCCCAGCTGGACGCAGTG
GTTCGTGGTGGCGCTGCCCGTGTCTGCCGTGTGCAACGTGCTCATCTGGGGGCTGCTGCTGGCGGTGTACCAGCCGCACAAGC
ACATCAAAGAGGTGCGGCCCATCCGCGCCCTCCAGGACGCCTTCACGCTGCAGCAGGTGGTGGTGGTGCTGGTCAGCCTGCTC
ACCGTGACGCTGTGGTGCCTCAATGGCATGCTGGAGCCCTACCTGGGGTCCATGGGGGTCATCGCCATCCTGCCGCTGGTGGC
CTTCTTCGGGTTCGGCATTCTGACCAAGGACGACTTCAACGCCTTCCTGTGGAACGTGGTCATGCTGGCCATGGGGGGGCTGG
CGGTCGGGGAGTGCGTGAAGAGCTCGCACCTACTGCAGTCCATCGCGCGCGGCATCCAGGACACGACTGCCGGCTGGTCCCTG
TACTGCGTGCTGGCCATGTTCTGCGCGCTGGTGCTGTGCTGCACGACCTTCATCTCACACACGGTGGGCGCATTTGTGATCCT
CCCTGTGCTGCAGAGCGTGGGAGACGAGATGGCGGCGGCCGGACAGCCGAACCACTCCAAGCTGCTGGTCATGGCGGCAGCTC
TGATGTGCTCCGGCGCCATGGGCCTGCCGGTCAGCGGCTTCCCCAACATGAACGCCGTGGCGCTGGAGGACCAGGCGGGGTTC
AATTACGTGGCCACAATCGACTTCATCAAAGTGGGGCTGCTCAGCTCAGGGTTTGCGTACGTGGTCATTATCTCGCTCGGATA
TTTACTGATGCTGATGGTGGGCTTT
>Chlamydomonas_sp.-M2_762.PTC1
CCAGCGCGCAGGCTGGACAAGCTGGACGCAGCCATCGCAAAGCTGGTGGACCTGCACGCGGTCATCTACCTGGCGGGTGATGC
CACCAAGGCCAAGGACCAGCTCTCCCGCGTGCTGCGCGACGTGGAGCGCAACACGGTGTGGCGCGACATGGTCGCCATGGAGC
GGCGGGCTGTGAGCGCCACGGTGGAGGGCACCAAGCGGCCGCCATGGTGGAAGGGCTACACGGAGCACATGGGGCTGGTGCTG
AGCGTGGCTGTCTTCGCGGTGCTTCTCTCGGTGGAGATATTCGACGAGGAAGAGAAGAACAACTGCCTGGCGCTGCTGGCCTT
TGTGTCCATGCTGTGGGCCACGGAGGCCATCCCGCTCTTCGCCACCTCCATGCTGGTGCCGCCCCTGGTGGTCATCCTGAGGG
TGCTCGTGGACCGCACCAAGGACCCCCCCGTGCGCCTCACAGCGCAGCAGGCCGCGCCCACCATCTTCCACGCCATGTTCTCG
CAGACCATCATGCTGCTGCTGGGCGGCTTCGCCATCGCCGCCGCCCTCTCCAAGCATTTCATCGCC
>Chlamydomonas_noctigama.PTC1
ATGAAGTTCACCCACCAGCTCAAGTTCAACACAGTGCCGGAATGGAGGGACCATTACATCCACTACGCGGCGCTGAAGAAAAT
CATCTACGCCATCGCCAAAGCCGAGGCTGACGAGCATCAGCATCCAGCGGGCCATGACGACGAACACCTGGGCGTCGCACTGC
TGGATAAGGTTGAGGCCACTGAGGAGTACCTGATCAAGAGCCTGGACAAGGAGCTGGCTGAGGTCATCAAGTTCTACATGGCC
AAGGAGGCGGAGATCCTGGGCAAGCTGGAGCAGCTGGACCTGGAGGTGCACAGCCTGGAGCAGCGCAGTGCTCTGGGCACGAC
GCTGCGGTCGACGTCGATGCCCCTGCCCAGCGATGCTGTGCCTGTGATCCTTGAAGAAGATGACCTGTCTCGCACCGAGTCGG
TGCGTGCCTCCCGCACCGAGTTCTGGCGCACCAACAGCCGCAGCCTCAAGCCCACCTCCAGAGCCCTCATCAAGGACAGCGGC
AAGATGAAGCAGCGCATCATAGACCTGTACAGCTCTCTGCACGACCTGGCGGACTTCCTTAACTTCAACAAGGAGGGCTTCCG
CAAGATACTGAAGAAGCATGACAAGGTGACCAGCAGCAACCTGAAGGACCGCTACTGGAGGGTGGTGGAGGACAAGTACCCCA
GCAAGAAGGCAGAGGTGCTGGAGCAGGCCATGGACAGGCTGACCGACCAGTTTGCAGTGCTGTACCTGCAGGGTGACACAGTG
AAGGCCAAGGACACCCTGGGCAGGGTGCTGAGGGAGCAGATCAAGGTTGAGCGCAACACTGTGTGGAAGGACATGGTGGCCAT
GGAGCGCCGCACAGTGGCAGCTGTCATCAAGCCGGGTGCAGCGGAGCCCAAGAAGGTGTCCTTCTTCGCCAAGCACCACTCCC
GGATCATGCTGCTGCTCTCCGTGGTGGTCTTTGCATCGCTGCTGTCGGTGGAGATCTTCCCGGAGCCTGAGAAGCAGAACTGC
CTTGCCATGCTGGTGTTTGTGTCCCTGCTGTGGGCCACTGAGGCCATCCCCTTGTATGCCACCTCCATGCTGGTGCCCCCCCT
GGTGGTGCTGCTCAAGGTGCTGGTGGACCGCAGTCACGAGGAGCCCATCCGCATGACAGCGCAGCAAGCGGCCCCCACCATCT
TCCATGCCATGTTCTCCCAGACCATCATGCTGTTGCTGGGTGGCTTTGCCATCGCCGCCGCCCTCAGCAAGCACTTCATCGCC
AAGCAGCTGGCCATCGCCGTGATGTCACGCGTGGGGCGCAAGCCTCACAACGTGCTGCTGGCCTCCATGTTCGTCGCCACCTT
TGCGTCGATGTGGATCAGCAACGTCGCGGCGCCGGTGCTCACCTTCTCCATCGTCATGCCCATCCTCAAGACCCTGGAGACCA
GCAGTGCCTTTGCCAAGTCCATGGTCATGGGCATCGCCCTGGCCTCCAACATTGGGGGCATGACCTCGCCCATCAGCAGCCCC
CAGAACATCTTTGCCATCGAGCGCATGTCCATGGACGGGCAGCCCCCCAGCTGGCTCTCCTGGTTCGCAGTGTCACTGCCGGT
GTCCAGCGTGTGCATCATCCTGTGCTGGCTGCTCATCCTGGCAGTGTACCAGCCCTGGCGCAGCGTCAGTGACGTGCGCCCCC
TCAAGCCCAACACCGACCCCATGAACATGACACAGGTGTTTGTGATTGTGATCTCCATGGCAACGGTCGGCCTGTGGTGCGCC
AACACAGCCCTGCAGTCGTACACTGGGGAGATGGGTGTGGTGGCGATGCTGCCGCTGGTTGCGTTCTTTGGCTTTGGAGTGCT
CAGCAAGGATGACTTCAATGGCTTCCTGTGGAACGTGGTCATGCTGGCCATGGGGGGCCTGGCTCTGGGGGAGGCTGTCAAGA
GCAGTGGTCTGCTGCAGTCCATTGCTGAGGCCATTAAGGAGGTCACTGATGGATATGACTTGTACCAGGTCCTGCTGGTCTTC
TGTGTCATGATCCTCGTCTGCACCACCTTCATCAGCCACACCGTTGGTGCCATGGTGATCCTGCCCATCGTGCAGAAGGTCGG
GGAGGACATGCCTGGGCCCCACCCCAAGCTGCTGGTCATGGCCGCTGCCCTTATGTGCTCAGGTGCCATGGGCCTGCCCGTGA
GTGGCTTCCCCAACATGCAGGCTGTGTCCCTGGAGGACTCCACTGGGCAGAACTATGTGGACACCCTGGACTTCCTCAAGGTC
GGTGTGCCCGGCTCCGTACTGGCATACCTTGTCATCGTATCACTTGGCTACACGCTGATGCTCCTGGTCCGCTTT
>Carteria_crucifera.PTC1
CAGGTGTATGTGATCGTTGTGAGTGTGGTGACTGTTGTGCTGTGGTGCCTCAACAGTGCCCTACAGAATGTGACGGGTGAGAT
GGGAGTCATTGCCATCATACCCATGGTGGCCTTCTTTGGCACTGGGGTGCTCAGTAAGGATGACTTCAACGGATTCTTGTGGA
ACGTGGTGATGTTGGCCATGGGCGGTCTAGCCATGGGAGAAGCGGTGAAGAGCAGCGGGCTGTTGGCAGCTATTGCTGAGGGC
ATCAAAGAATTGGTGGCAGGGATGGACTTGTGGGAGGTGCTAGCGATATTCTGCAGCCTCATCCTGGTCTGTACCACCTTCAT
CAGCCACACGGTGGGGGCCATGGTGATCTTGCCCATCGTGCAGTCAGTGGGGGAGATGGCGCTGGGCCACCCTCACCCCCGCC
TCTTAGTCATGGGGTCAGCGCTCATGTGCAGTGGCGCCATGGGCCTGCCCGTGTCAGGCTTCCCCAACATGAACGCAGTGGCT
CTGGAGGACTCCACTGGGGTCAACTACGTTAGCACTGTGGACTTCTTGTGGGTGGGTATCCCCAGCAGCATCTTTGCTTACGT
GGTCATTGTGACAGTCGGCTACTTCCTCATGCTCATGGTCAGATTC
>Volvox_aureus-M2242.PTC1
TGCTTGGCGTTGCTGGTGTTTGCATCTTTGTTGTGGTCACTGGAGGCCGTGCCTCTGTACGTAACAAGTATGGCTCTACCTTT
TCTGATTGTCGCCATGGGTGTCCTTGTTGACCACCCAAATGATTCCAAGGATCCACCTAAACGCCTCACACCGCAGCAAGCTG
CACCAGCGATCTTCCATGCCATGTTCTCACAGACAATCATGTTACTTCTTGGTGGTTTCTCGATATCAGCCGCTCTGTCCAAG
CACGCCATCGCAAAGCAGGTGGCTGTGGCTATTTTGTCTCGTGTTGGAAGAAAACCACGCAACGTACTGCTTGCTGCAATGTT
TACAGCCACATTCGCATCAATGTGGATTTCCAATGTGGCGGCACCAGTACTCTGTTTTGGCCTAATTCAACCCATCCTGAGGA
CCCTGGACCCTGGACATCCTTTTGCGAAGTCCCTGGTTATGGGCATTGCGCTGGCTTCCAATGTCGGAGGAATGACGTCACCC
ATATCTTCACCCCAAAACATTTTTGCAATAGAGCGCATGTCGATGGATGGACAGCCGCCCAGCTGGCTCTCTTGGTTCGCTGT
AGCAATACCAGTTTCCATCACATGCAATTTTCTCTGTTGGGCCTTACTCCTTCTGGTCTACCAACCAGGACGAGCTTTGGGTG
AGGTCCGACCGTTAAAACCCAACACGGACCCCATCAACGGGACACAGGTGTACATCATCGTTGTGTCGATGTTGACTGTGGCA
GCCTGGTGTGCGAACACATTCCTCCAGAGGTATACTGGCGAGATGGGCATCATCGCCATTGTCCCATTGGTAGCGTTTTTCGG
TTTCGACGTACTGAATAAGGATGATTTCAACTCGTTTCTCTGGAACGTCGTCATGCTGGCCATGGGCGGGTTGTCTCTAGGTG
AAGCAGTTAAGAGCAGCGGTTTGCTTGCAGCGCTCACAAACAGTATTAGTGAACTGGTAACAGGCTTCACTATGTTTCAAGTT
ACCCTCATATTCTGTGGCCTGGTGCTGCTGGCGACGACGTTTATCAGCCACACAGTAGGGGCTATGGTTATCCTACCAATCGT
GCAGAGTGTTGGAGAGAGTATGGCGGGGACACCACACCCTAAGCTGTTGGTCATGGCATCGGCGCTCATGTGCTCTGGTGCTA
TGGGCTTGCCTGTCAGCGGATTTCCGAACATGAACGCTGTCAGCTTGGAGGATAGCACTGGAAATGCGATTGTCAGTACTAAG
GACTTCCTGTACGTCGGTGTGCCTTCATCGGTGATGGCTTATGGCATAATCGTCAGTCTAGGGTATGTGCTCATGCTGTTGGT
AGGTATG
>Phacotus_lenticularis.PTC1
ATGAAGTTCACGCATCAGCTCAAGTTTAACTCGGTCCCAGAGTGGCGGGACCAGTATGTTGACTACGCCCACCTCAAGCGCTT
CATCTATGCCATTGCCCGCGCCGAGCAGGATGACATCCAGCAGCTGCACGAGGTGCACGACACCACGATGCCTCTGCTGCCCC
ACACCGTCACCATGGGTCATGACAAGGTGGAGGCCACGGAGGAGAACCTGCGCCAGGCGCTGGACAAGGAGCTGCAGCGCGTC
ATCTCCTTCTACATGGCCAAGGAGGCGGACATCTTGGCCAAGGTGACGGCGCTGGAGCTGGGCATCCATGCGCTGGAGAAGCT
GCCCGCCCGGGGGGTCAGTCTGGAGCTGGACCCCACCCGGCAGGGCAGCCAGGTGGCGGCTGGGGGCGTTGCGGGAGGGGGGG
CCCCCCCAGGGGGGCGCCACGTCCCCCTACTGCAGGGCGCCCCCTCGGTCACGCGCGAGGGCTCGGGCGGCATTGCGCACTCC
ATCTCCCCCCAGCAGTCCTCCTCCTCCCCCCAGCTGGGCGGCCCCGGCAGTGGCCGCACCGCCGGCACCGGGGGCAGTCAGTC
CGCCTCCCCCCAGCCCTCGGGAGCCATGCACGGGGGGGACCTGGAGGCCCCCCTGCAGGGGGGCGACCACACCGGCAAGACCT
CCCCCCACTGGTCACGCGCCGCCCGCGTCGAGTTCTGGGGGGGGGCCCAGCCCGCGCACCGCTTCACCGGGGGAGCCTCCTTC
TCAGCCGCCTCCTTCTCGGGGCCCTTCGTGCGCGACATGCAGGCCCACAAGGAGAGGCTGCGCCCCCAGTTCAGCGACCTCTA
TCTCTCCCTGCACGACCTCCTGGGCTTCCTCAGGCTCAACAAGGAGGGCTTCCGCAAGATCATCAAGAAGCACGACAAGATGA
CCAGCAGCAACCTGAGGGAGCAGTACTGGCCGCTGCTGGAGGCCAAGTACCCCATCCAGAGGGCCGAGCTACTGGAGGCCACC
ATCGCCTCCCTGGTGGACCACTACGCCGTCATCTACCTGGGGGGCGACGTGGGCACCTCCAAGGCCCACCTGGACAAGGTGCT
GCGTGACCAGATCCAGGTGGAGCGCAACACAGTGTGGAGGGACATGGTGGCTCAGGAGAGGCGCACCACAGCGGCCGTGGTGG
CCACCACGTACAAGCAGAAAGTGTGGGCCAAGGTGACGCCCCACATCGCGCTGGTCTCCAGCGTCGCCGTCTTTGCCCTGCTG
CTGTCTGTGGAGGACCTGTTCCCCGAGGCGCCCGAGAAGCAGAACTGCCTGGCGCTCCTAATCTTCGTGTCCATGCTGTGGGC
CACCGAGGCGGTCCCCCTCTACGTCACCTCCCTGGCCATCCCCCTGCTGGCGGTCACGCTGAGGGTGCTGGTGGACAAGACCA
CCGACCCCCCCCAGCGCATGCCGGCGCAGCAGGCCGCCCCCGCCATCTTCCACTCCATGTGCTCCCAGGTGATCCTGCTGCTG
CTGGGTGGCTCGGCCATCGCCTCGGCCCTCACCAAGCACTTCATTGCCAAGAAGCTGGCGCAGGTGGTGCTGGCCCAGGCCGG
CCGCCAGCCCCACAATGTGCTGCTGGCGCTCATGCTGGTGGCAACCGTGGCCTCCATGTTCATCTCCAACGTGGCCGCCCCCG
TGCTCTGCTGGTCCCTGGTGGAGCCCATCCTCAAGTCCTTTGACGCCGACAACCCCTTCTCCAAGTCGCTGGTTATGGGCATC
GCGCTGGCCTCCAACATTGGCGGCATGACCTCCCCAATCTCCTCCCCCCAGAACATCTTTGCCATTGAGCGCATGGGCATGGA
CGGGCACCCCCCCTCCTGGCTCTCCTGGTTCGCGGTGGCGCTGCCAGTCTCCTTCATCTGCATCCTGGTGTGCTGGGGCCTCA
TCCTGGGGGTGTACCGCCCCTGGTCCAAGGTGGCGGAGCTGAGGCCCCTCAAGGCCAGCGCCGACAAGGTCACCTTCACCCAG
ATCTACGTGGTGCTGGTGACGGTGGTGACGGTGGGGCTCTGGTGCTGCAACACGATGCTGCAGCCGTACACGGGCGAGATGGG
CATCGTGGCCACCATCCCCATCATCGCCTTCTTTGGCTTTGGGGTGCTCAACAAGGACGACTTCCTCTCCTCCCCCTGGCTGG
TCATGACGCTGGCCATGGGCGGCCTGGCGCTGGGCGAGGCGGTCAAGAGCAGCGGGCTGCTGCTGTCCATCGCACACTCCATT
GGCGACCTGGTGCAGGACCTGGACCTCTTCACCGTCTGCGTCATCTTCTGTGGCTTAGTCCTGGTCTGCGCCTCCTTCATCAG
CCACACGGTGGGCGCCATGATCATCCTGCCCATCGTGCAGTCGGTGGGCGAGCAGATGCCGGGCCCCCACCACTCCAAGCTCC
TGGTCATGGCCTCGGCCCTCATGTGCTCGGGGGCCATGGGCCTGCCCGTGTCGGGCTTCCCCAACATGTTCCTCATCTCCAAG
GACGACGGCACCGGCAAGAACTACGTCAACACGCTGGACTTCATCAAGGTGGGCGTGCCCGGCTCAGTGGGCGCCTTCTTCGT
CATCGCCACCGTGGGCTACGTCCTCATGCTCATGGTG
>Stephanosphaera_pluvialis.PTC1
AGGAGACAGAGAGTGCGCTTCTGGGCTTCTCTAGACACCAGGGCAGAGCTCCGGGACTTGCGGCTGGTGCGGGGCATGATGCG
CTACCGCTTCAACGACATCTACACAACACTGAATGACCTCATGGAATACATCATGCTGAACCGCGAGGGGCTCCGCAAGGTGG
TGAAGAAGCACGACAAGCTGACCACAACTGTGGCGCTCAAGGAATCCTACTGGCCGACTGTGGACCAACAGCTGGCGCTGAGC
AAGAGGGATGCAATGGCCCAGCAGATAGAGCAGCTGGTGGACCTGTACGCGGTGATGTTCACAGCAGGGGACGTGGACGCTGC
GAAGGAGCTGCTGAGCAAAAACTTGCGCGAACACATCAAGGTGGAGCGCAACACGGTGTGGCGGGACATGGTGGCCCTTGAGC
GTCGCACAGTGGCTGCCACAGTGCAGCAGACCACAGGCAAGGCCGCAGCCAAATTGCAGAGGTACAGGGAGCCCTTGTGTCTG
CTGCTGTCCCTTGCTGCCTTCTTCGCCCTGCTGCGGGCAGCCCCCTTTGCTGAGCCAGAGAAGAACCAGTGCCTGGCCCTGCT
GGCGCTGTGCTCCCTGTTGTGGGCAACAGAGGCAGTGCCCCTGTTCGCCACTGCCCTGGCCATCCCCCCTTTGGTTGTGGTCA
TGCGGGTGTTGGTGGACAGGAGTGACCCTGCAGCGCCACACAGGCTGACGCCCCAGCAGGCCGCCCCTGCCATCTTCCACGCC
ATGTTCTCTCAGGTCATCATGCTGCTGCTTGGCGGGTTTGCCATCGCTGCCGCACTCTCCAAGCACTTCATTGCAAAGCAGAT
GGCAGTGGCAGTCTTATCCCGTGTGGGACGCAAGCCACACAACGTGCTGCTGGCTGCCATGTTTGTGGCCACCTTTGCGTCCA
TGTGGATCAGCAACGTGGCCGCCCCTGTGCTTTGCTTCTCCCTTGTGCAACCCATCCTGCGGACCATGGACGTGACAACGCCA
TTTGCCAAGAGCCTGGTGATGGGCATCGCGCTGGCATCAAACATAGGTGGCATGACCTCGCCCATCTCCTCGCCCCAGAACAT
TTTTGCCATAGAGAGGATGGGTATGGATGGGCACCCGCCCAGCTGGCTTGCATGGTTTGCAGTAGCCCTTCCCGTCGCCATCA
TCAGTAACCTGCTGGCTTGGGGCTTGTTGCTGCTGGTGTACCGGCCATGGACACACACCACAGAGGTCCGCCCCCTCAAGCCC
AGCTCCGACCCCATCAACCTGACGCAGGTGTATGTGTGCCTGGTGAGTCTGGCAACAGTGGGTCTGTGGTGCGCCAACACAGC
ACTGCAGAAGTACACTGGGGAGATGGGTGTTGTGGCAGTGCTACCCCTTGTGGCCTTCTTTGGCTTTGGTGTGCTGAACAAGG
ACGATTTCAATGGCTTCCTTTGGAACGTTGTCATGTTGGCCATGGGCGGCTCAGCACTGGGTGAGGCTGTCAAGAGCAGCGGG
CTGCTGGTGTCCATCGCTGAGAGCATCAGGCAGCTGGTGGCAGGCATGGACCTGTGGATGGTGACGGTGGTGTTCTGCCTGGC
AGTGCTGTTCTGCACCACCTTCATCTCACACACAGTGGGAGCCATGGTCATCCTACCAATTGTGCAGAGCGTGGGGGAGGCCA
TGCCCGGGCCGCCCCACTCCAAGCTGCTGGTCATGGCCTCTGCGCTCATGTGCTCAGGTGCTATGGGCCTGCCAGTCAGTGGC
TTCCCCAACATGAATGCAGTGTCACTGGAGGATGCTACAGGGCAAACGTATGTCAGTGCCAGTGACTTCATTGCAGTGGGAGT
GCCATCCAGCGTAGCAGCATATGCAGTCATAGTCACAGTGGGGTACAGTCTGATGCTGCTGGTTGGGTTC
>Chlamydomonas_eustigma.PTC1
ATGAAGTTCACTCATCAAATTAAGTTCAACAGTGTTCCCGAGTGGAGAGACCACTACATCGACTATGCCCACCTGAAGAAAAT
CATATATGCAATCGCAAAAGCTGAGGCTGATGAGCAGCAGCAACACCATCTAGATGAGGAACATCCTCTTCTTACCAGACAAC
AGACTGCTCATGGAGAGAAGGTTGAAGCTACTGAGGAGGCTTTGATCCAGGCACTTGATAAGGAGCTTGCCAAGATTATCAAA
TTCATTATGGCCAAAGAGGCTGAGACCCTGGGGAAGCTAGCTCAGCTGGATCTAGAGGTTCACAGTCTTGAGGCCCAGCGGGT
TGGAAGTATGTTCACACCTCCCATTGTGAACAGGTTCACATCACTTCAAGATGCAGGGAATACACGTCTTGGAGGCTCACTGC
CAGATCCCCAAAAAGATGGTTTTGAAACTCTAGGACTAGCCGACAGAAGACCCTCTGAGGTTATGGAGGAGGCTGTACGCCCT
GATCTGGAAGGGGGTATTGGCTCAAACTCTTTCCGTGCCTCCCGAGTCCATTTTTGGCACTCAAACAGTCTGCCTGCTACCAC
TCGCACTGGTGCTCGAGTACTGGCTAAGGACAGTGCTAAGATGAAACCAAGAATCACGGACCTGTTTGTTGTGCTGCATGACT
TGAAGAACTATCTGTCCTTAAACAAGGAGGGCTTCAGAAAGATTTTGAAGAAGCATGACAAGATGACTAGCAGTAATCTGAAG
AGCAGGTATTGGTGTATCATTGAGGAACAGTACCCCAGCAAGAAAGAGGAGGGCATCATGCAAGCTATCAACAAGCTAGTGGA
TCTGTATGCTGTGCTCTTCCTGAAGGGTGATTTTGAGAAGGCATCAAGCGTCCTTAATCGTGTGCTAGGAGAACAGATTAAGG
TGGAGAGGAACACTGTGTGGAGGGATATGGTGGCAATGGAGCGCAAGACTGTGAATGCTGCAGTTCATAAGCCACAAGGTGTG
GCCACTCGTGTCACCTGGCTGCAGCAGAACATGAAGCACATCCTGTTGATGCTTGCCGTCCTGACCTTTGCCACACTTCTGAC
AGTGCAGACCTTTGAAGAGCCAGAGAAGAACAACTGCTTGGCTATGCTGGTGTTTGTGTCCATGCTGTGGGCCACTGAGGCCA
TCCCTCTGTTTGCCACCTCCATGCTGGTGCCACCCTTAGTGGTCATCCTCAGGGTTATGGTGGATCACACAAAGTCACCTCCA
GAGCGCATGCCTGCGAAGGATGCTGCACCTGCCATCTTCCACTCCATGTTCTCACAGGCAATCATGCTCTTGCTGGGTGGTTT
CGCTATTGCTGCTGCCCTGAGCAAGCACTACATTGCCAAGCAGCTAGCTATCTCGGTCATGTCCAGGGTTGGACGTAAACCCC
AGTTTGTGATCCTGGCCGCCATGTGTGTGGCAGCCTTCGTGTCCATGTTCATCAGCAATGTAGCTGCGCCTGTACTCACGTAC
TCTATTGTCATGCCCATCTTGAAGACCTTGGATACAGGCTGTCCCTTTGGTAAGGCCTTAGTGATGGGTATTGCCCTGGCATC
GAATGTCGGAGGGATGACTTCCCCCATCAGCTCTCCTCAGAACATATTTGCGATCCAGCTCATGTCAAACGACAGCAACCCGC
CCAGCTGGCTGGCCTGGTTTGCCATCTCCCTGCCAGTATCGGCTCTTTGTGTCTTGATGTGCTGGTCCCTGATCTTGATTGTG
TATCAGCCTTGGCGCCGCGTGGCCGAGGTGCGTCCCCTAAAGCCCAGCACTGATCCCATCAACGGCACCCAGGTCTATGTCAT
CATCATCTCCCTGGCTACAGTGGCTTTGTGGTGTGCCAACACCGTCCTCACACCGTACACTGGGGAGATGGGGGTTGTAGCAG
TGTTGCCGCTGGTTGCCTTCTTTGGTTTCGGGGTGTTGAGCAAAGAGGACTTCAATGGGTTCTTGTGGAATGTTATCATGCTG
GCCATGGGAGGCATGGCTGTGGGAGAAGCTGTAAAAAGCAGTGGCCTCCTCCACTCCATTGCGCTGGGCATACAGGATCTGAC
CTCAGGTCTTGATCTCTTCCAGGTCATGATCATCTTCTGCCTCCTGGTTCTCATTTGCACAACCTTCATCAGCCACACGGTGG
GTGCCATGGTCATCCTGCCTATCGTCCAGAGTGTGGGAGAGTCCATGCCGGGCACAGCGCACCCCAAATTACTGGTTATGGCA
ACCGTCCTCATGTGCTCTGGAGCTATGGGCTTGCCAATCAGTGGTTTTCCAAACATGCAAGCTGTTTCCCTGGATGATGGCAT
GGGGCAGAACTATGTCAGCACCATAGACTTCCTTATGGTGGGAGTCCCCAGCTCTGTGCTAGCTTACTTCGTCATAGTCTCAG
TGGGATACTCTCTAATGCTCCTAGTGCGCTTTTGA
>Chlamydomonas_incerta.PTC1
ATGAAGTTCACTCACCAACTTAAGTTTAATAGTGTGCCGGAATGGCGAGAACACTATATACAGTATGGACACCTAAAGAAATA
TATTTATGCGCTTGCAAAGAGGGAAGCAGACCTTCAAGCTGGCGGCCAGGAAGAGGAGGCGCTTCTCGCCCCGCTGCTGCTGG
AAGCGGGGCGCGATCAGGGCCCCACGGAGGAGGGCTTCCAGCAGGAGCTGGATGCGCAGCTCGCAGCCACGTTGAGCTTCTTC
GCGGTGAAGGAGGCTGACCTGCTCGCCAAGGTGTCGGCGCTGGAGCTGGACATCCAGAGCCTGGAGAAGATCCCCAACCGCGC
CGAGGCCTCCACCCTGGCGCGCATGGGCATGGGCATGGGCGGCAGCGCCAGCCCCGGCGGCCCCATGAGCAGCCCGCGCGCCG
CCGCCGCCGCCGCCATGTCGGCCGTGGCCTCGCTGGTCAGCCACAGCCCCTCCACACTGGACCTGGCGCGCATGGTCAACAGC
ACGCCGCCCGAGGACCACCGCAAGGTGCGGGTCAAGTTCTGGGAGAACCCGCCGCGGCACCTGTTCAGCACCAACCTCAGCGC
GCGCCGTGCCAAGCTGCAGGCGCGCTTCCAGGACCTGTACATCTCGCTACACGACCTGCGCGAGTTCCTGCACATCAACAAGG
AGGGCTTCCGCAAGATCATCAAGAAGCACGACAAGCTGACGCGCGCCGTGGACCTGCGCGCGCGCTGGTGGCCCAACGTGGAG
GCGCACCTGGCGCCCGCCGCCAAGCAGGCGGAGCTGGACGGTGCCATAGCCGCGCTGACCGACCACTACGCCGTGCTGTACAC
GCGCGGCGACGTGGCCCAGGCGGAGGAGCAGCTGTCGCGCGGGCTGCGGGAGCACATCACCGTGGAGCGAAACACCGTGTGGC
GCGACATGGCGGCCATGGAGCGCAAGTACGCGGCGGTGTCGGTGAAGCAGGCGGCGGCGCCCGGGGCGCGAGTCACGTGGCTG
CGCACGCACGCGCGCTGGCTGAAGCTAGCGCTGAGCGTGGCGGTGCTGGTGGTGCTGGCCAACGTGGAGGTGTGGCCGGGGCC
CGAGAACGAGCCGCGCAACAACTGCCTGGCGCTGCTGGTGTTCGCGTCGCTGCTGTGGAGCCTGGAGGCCGTGCCGCTGTTCG
TGACCAGCATGGCGCTGCCGCTGCTGATCGTGGCCATGGGCGTGCTGGTGGACCGCAGCAAGGACCCGCCGCAGCGCATGAGC
CCGCAGCAGGCGGCGCCAGCCATCTTCCACGCCATGTTCTCGCAGACCATCATGCTGCTGCTGGGCGGCTTCTCCATCGCCGC
CGCGCTGTCCAAGCACGCCATCGCCAAGCAGGTGGCTGTGGCCATCCTGTCGCGTGTGGGCCGCAAGCCGCGCCACGTGCTGC
TGGCGGCCATGTTCACCGCCACCTTCGCCAGCATGTGGATCAGCAACGTGGCCGCGCCCGTGCTGTGCTTCGGCCTCATACAG
CCCATCCTCAGGACGCTGGACCCCGGCCACCCCTTTGCCAAGGCGCTGGTGATGGGCATCGCGCTGGCCTCCAACGTGGGCGG
CATGACCTCGCCCATCAGCAGCCCGCAGAACATCTTCGCCATCGAGCGCATGAGCCTGGACGGCAGCCCGCCCTCCTGGCTAG
CCTGGTTCGCGGTGGCGCTGCCCGTGGCCGTGGCGGCCAACTTCGTGTGCTGGGGGCTGCTGCTGCTGTGCTACCAGCCCGAC
AAGGCCATCGCCGAGGTGCGCCCCATCAAGCCCAACACCGACCCCATCAACGGCACCCAGGTGTACATCATCGTGGTGTCGCT
GCTGACGGTGGCGGCCTGGTGCGCCAACACCTTCCTGCAGCGCTACACGGGCGAGATGGGCGTGATCGCGGTGGTGCCGCTGG
TGGCGTTCTTCGGCTTCGACGTGCTCAACAAGGACGATTTCAACAGCTTCCTGTGGAACGTGGTCATGCTGGCCATGGGCGGC
CTCAGCCTGGGCGAGGCCGTCAAGAGCAGCGGCCTGCTGGCGGCGCTGGCGCTCACCATCAGCGACCTGGTCACGGGGCTCAG
CCTGTGGCAGGTGGCCACCATATTCTGCGGCATGGTGCTCGTGGCCACCACCTTCATCAGCCACACCGTGGGCGCCATGGTCA
TCCTGCCCATCGTGCAGAGCGTGGGCGAGGCCATGCCCGGCACGCCGCACCCCAAGCTGCTGGTCATGGCGGCGGCGCTCATG
TGCTCCGGCGCCATGGGCCTGCCGGTGAGCGGCTTCCCCAACATGAACGCGGTCAGCCTGGAGGACAGCACCGGCAACGCCAT
CGTGGGCACCGGCGACTTCCTGGCGGTGGGCGTGCCCAGCTCCGTGTTCGCGTACGGCATCATCGTCTCGCTCGGCTACCTGC
TCATGCTGGCGGTGGGCTTCTAG
>Chlamydomonas_schloesseri.PTC1
ATGAAGTTCACACACCAACTCAAGTTTAATAGTGTGCCGGAATGGCGAGAACACTATATACAATATGGGCATTTGAAAAAATA
CATTTATGCGCTTGCTAAGAAGGAAGCGGACCTGCAAGCTGGCGGCCACGATGACGAGGAGGCGCTGCTTGCTCCGCTGCTGG
AAGCAGGACGTGATCAGGGCCCCACGGAGGAGGGCTTCCAGCGTGAGCTGGATGCGCAGCTCGCGGCCACGCTGAGCTTCTTC
GCGGTGAAGGAGGCCGACCTGCTGGCCAAGGTGTCCGCGCTGGAGCTGGACATCCAGAGCCTGGAGAAGATCCCCAACCGCGC
CGAGGCCTCCACCCTGGCGCGCATGGGCGGCCCCGGCAGCGCCATGGCCAGCCCCGGCGGCGGCGGCCCCATGGCCAGCCCGC
GCGCCGCCGCCGCCGCCGCCATGTCGGCGGTGGCGTCGCTGGTCAGCCACAGCCCCTCCACCCTGGACCTGGCGCGCCTGGTC
AACAACACGCCGCCGGAGGACCACCGCAAGATCCGCGTCAAGTTCTGGGAGAACCCGCCGCGCCACCTGTTCAGCACCAACCT
CAGCACGCGTAGGGCTAAGCTGCAGGCGCGCTTCCAGGACCTGTACATCTCGCTGCACGACCTGCGCGAGTTCCTGCACATTA
ACAAGGAGGGCTTCAGGAAGATCATCAAGAAGCACGACAAGCTGACGCGCGCCGTGGACCTGCGCGCGCGCTGGTGGCCCAAC
GTCGAGGCGCACCTGGCGCCCGCCGCAAAGCAGGCGGAGCTGGACGGAGCCATCGCGCAGCTGACGGACCACTACGCGGTGCT
GTACACGCGCGGCGACGTGGCGCAGGCGGAGGAGCAGCTGTCGCGCGGGCTGCGTGAGCACATCACCGTGGAGCGAAACACCG
TGTGGCGCGACATGGCGGCCATGGAGCGCAAGTACGCGGCCGTGTCGGTGAAGCAGGCGGCGGCGCCAGGGGCCAGGGTCACG
TGGCTGCGCACGCACGCGCGCTGGCTCAAGCTGGCGGGCGCCGTGCTTGTGTTCCTGGTGCTGGCCAACGTGCAGGTGTGGCC
GGGCGCCGAGAACGAGCCGCGCAACAACTGCCTGGCGCTGCTGGTGTTCGCGTCGCTGCTGTGGAGCCTGGAGGCCGTGCCCC
TGTTCGTGACCAGCATGGCCCTGCCGCTGCTGATCGTGGCTCTGGGCGTGTTGGTGGACCACACCAAGGACCCGCCGCAGCGC
ATGACGCCGCAGCAGGCCGCGCCCGCCATATTCCACGCCATGTTCTCGCAGACCATCATGCTGCTGCTGGGCGGCTTCTCCAT
CGCCGCCGCGCTGTCCAAACACGCCATCGCCAAGCAGGTGGCTGTGGCCATCCTCTCGCGTGTGGGCCGCAAGCCGCGCAACG
TGCTGCTTGCGGCCATGTTCACTGCCACCTTCGCCAGCATGTGGATCAGCAACGTGGCAGCGCCCGTGCTGTGCTTCGGGCTC
ATCCAGCCCATCCTCAGGACGCTGGACCCGGGCCACCCCTTTGCCAAGGCGCTGGTGATGGGCATCGCGCTGGCCTCCAACGT
GGGCGGCATGACCTCGCCCATCAGCAGCCCGCAGAACATCTTCGCCATCGAGCGCATGAGCCTGGACGGCCGCCCGCCCTCCT
GGCTGGCCTGGTTCGCGGTGGCACTGCCCGTGGCGGTGGCGTGCAATTTCGTGTGCTGGGGCCTGCTGCTGCTGTGCTACCAG
CCCGGCAAGGCCATCGCCGAGGTGCGGCCCATCAAGCCCAACACCGACCCCATCAACGGCACGCAGGTGTACATCATTGTGGT
GTCGCTGCTGACGGTGGCGGCATGGTGCGCCAACACCTTCCTGCAGCGAGAAGTGCACAAATCCATTTATGCTACAACGGGCG
AGATGGGCGTGATCGCGGTGGTGCCGCTGGTGGCGTTCTTCGGGTTCGACGTGCTTAACAAGGACGACTTCAACAGCTTCCTG
TGGAACGTGGTCATGCTGGCCATGGGCGGCCTCAGCCTGGGCGAGGCCGTCAAGAGCAGCGGGCTGCTGGCGGCGCTGGCGCT
GTCCATCAGCGACCTGGTCACCGGCCTCAGCCTGTGGCAGGTGGCCACCATCTTCTGCGGCATGAGCGCAGCCAAGCTCTGGA
GTCCACCGGGGGGACGCTCCAACCAACCTGGACCACGCCAGCAACCTCAGAAGGGTTACTGCTGGTACAACAACGCAGGGCCC
AGCGGGCTCACCAACCACTAG
>Chromochloris_zofingiensis.PTC1
ATGAAGTTCAGTCAGACGTTGAAGTTCAATAGGAGACCCGATTGGGAGATCCACTACATCAACTATGCCCATTTGAAAAGGCT
AATCACGAAGGTGCAGCAGGCTGAATTTGCGGAGCAGAACAACCTGCCATTGCACTTCGGGGATGAGGAAGCAGGTGTCAGAT
CCCCTTTGCTCAGCCAGACGTCCTTCAACCGTCAGCAGTCTGTGTCAGCAGCTCTTACTCGCCAGCAGTCTTTCACCATATCA
GCAGCACAGTGCGATGAAGCATTCATCAAAGCGCTGGACAGCGAATTGGCCAGAATCATCCAGTTCTACATGCGCAAGGAGAG
TGAGTTGCTGGCAAGGTTTGAGTCTGCGGCTCTAAGGATCCACAGCATTGAAGGGCCAGCACTGCCAGGACCAGCTGCCTTGG
ATACAGCACAAAGGATCCAGTTTTGGTCACAGGACACAAAGGAGATTGCACTGGAGCGTGAGAAGCTTCGCTCTGAGATGACC
GACCTGTATGAGCAGCTGCATGCGCTCAGCAAATACTTAGAACTGAACTTCACAGGCTTCAGGAAGATCTTGAAAAAGCATGA
CAAAATGACATCCCAGAATCAGTACAAGGACTCCTACATGCCGATTGTGGAAGCCAAACTCCCATTGAAGAACCGTGAAATGA
TTTCTGGTGTCATCAACAACCTGGTGGAGATGTATGCGGTCGTGTGTACACGCGGTGATGTCAATCGTGCGCAAGCTGAGCTC
AAGCGCAAGTTGAAAGATGAGGTCGCGTTTGAACGGTCTACTGTGTGGAGAGACATGGTGGCTATGGAACGAAGGGGTGCTTC
TGTGGCTGTACATGAAGCTTCTAGTTTGGCTGATCAGCCAAAGAAGCCCCGTTGGTGGCAAGCACATAGACAACTACTGCTGG
TGACATTGTGCGTTACTGTGTTCGCTGTCCTGCTGTCTGTACCCATTTTCCAGCAGCCGGAGAAACAGAATTGCTTAGCTCTG
CTGGCGTTTGTGTCATTACTGTGGTGTACGGAGGCCATCCCCTTGTTTGTGACATCCATATTGGTGCCCTTGTTGATTGTGGT
GCTGCGTGTGTTGGTAGATCGCAGCGCAGATCCTCCCAGACGCCTACCGCCTCAGGAGGCTGCCCCAGCGGTGTTTCATGTCA
TGTTCTCACAGGTCATCATGCTCCTGCTGGGTGGCTTTGCTATAGCTGCAGCCCTCAGCAAGCACTTTATTGCCAAGCAGCTG
GCTGTAGCAATCCTCAGTCGGGTGGGCAGGAAGCCGCAGTATGTGCTGCTTGCCAACATGTTGGTGGCAACCTTTGCTAGTAT
GTGGATCAGTAATGTGGCTGCACCAGTATTATGCTTCTCACTTGTACAGCCCATTCTAAGGACCCTGTCCCCAAGTCACGCGT
TTGCTAAGAGCTTAGTCATAGGCATCGCATTGGCATCGAATCTGGGGGGAATGACGAGCCCTATCTCCAGCCCCCAAAACATT
TTTGCAATTGAGCGGATGAGCATGGATGGCAACCCTCCAAGTTGGTTGAGCTGGTTTGCTGTCGCACTGCCTGTGTCTGTGCT
GGGCAATCTGCTGTGCTGGGGCTTGATTCTGTTGGTGTATAACCCAGGAGCTACCATCAAAGAGGTTCGCCCCGTGAAACCGC
CAGAAGATCCGCTCAACGGCACCCAAATCTACGTCATCCTCGTCAGCGTGGCTACTGTTGGTCTATGGTGCTTCAATTCCTTC
ATACAACATGTGACAGGAGAGATGGGTGTGCTGGCGATCCTGCCATTGGTAGCATTCTTTGGCTTTGGAGTGCTGGATAAGGA
CGACTTTAATGGATTCCTTTGGAATGTGGTCATGCTGGCTATGGGGGGCTTGGCACTGGGTGAAGCTGTGAAGAGTTCAGGAC
TGCTGCTAACAATAGCAACGGGTATTCAGGACTTTGTGGCTGGTCTTGGCCTATGGTCCGTGCTGGCTGTGTTCTGCTTCCTG
GTGCTCATCTGCACCACCTTCATCTCACATACAGTAGGCGCCATGATCATACTGCCTATCGTGCAATCTGTGGGAGAAACAAT
GAGCGGCACGCCCCATCCCAAGCTATTAGTTATGGGCTCTGCGCTCATGTGCTCAGGGGCTATGGGGCTGCCAGTTAGTGGCT
TCCCTAACATGAATGCTGTGGCTTTAGAAGACCCCACCGGCCAAAACTACGTCAACACCATTGATTTCTTGAAGGTGGGTGTA
CCTGGTTCCATCATGGCGTATGGAGTGATTGTCAGTTTGGGGTATGTGCTGATGATAGCTGTTGGCATGTAA
>Coccomyxa_subellipsoidea.PTC1
ATGAAGTTTGGCGCGGAGAGGGCAGGCCACGCGCTGCTGAGCTGGCTGACTGCTGCCTGGCTGTGGCTTCTGCAAGCCTGGGA
GGTGGTCGCAGAGTGGGGCCGCCAGTGCTGGGGGGCTCTTTTGCATGCCTGGCATTATATCGCCAGCGCTGTCATGCAGGCAG
TGCACTGGCAAACAGAGAATCGAATCGCAGATCTTGGACGTATTCCAGAGGAGGTAGGAGGTGACCTGGACAGGACAATATCC
CTGGCTCTTGAAGAGGGCGGGGACGATATCAAGGGCGCATTTGACAGCGAGCTTAACCGCATTACTACTTTCCACAAAAAGAA
GGAAGAGGAGCTTCTGGGTGCAGTGGACAAGCTTGGGGAGGAGGTGAGCAGTGCTGTGGAGCCAAGTGCACAGCAGAGCGCTC
CTGATGCGAGCTCGCCGCTGCTTGGGACTTCCAGGAATGCGGAGGCACTGTACTGGGGCCAGGACACTGTTGCTGTGCGCATT
GCGCGGGAGCAGCTAAGGGAGACCTTTCAGGAGCTGTATGTGGAGATCCAGGGGCTGATAGATTTTGTGGAGGTGAATCGTAC
AGGCTTCAGGAAGGCCCTGAAGAAGCACGACAAGGTGTTGGGCGCGCTTGGGCACCCAAAGATGCAGCCAACGTACATGCCTA
ATGTCGAGGCTGCCTTCCCTGAGAAGAACCGCCTGCGTGTGTCAGAAGCTCAGAAGCAGCTGGTGGAGCTGTACGCTGTGGTG
TGCTGCCACAACAACCTGCTGCTGGCCCAGCTGGAGCTCAAGGCACAGCTGCGCTCCCAGCTCAAGCTTGAGAGGACGACAGT
CTGGAAGGACATGGTGGAGAAGGAGCGAAAGGAGAACGCTGCCACGGTCGACGACAGCGGCGCTGAGTCCAAGCCCTGGTACC
GCAGCAGCCTCTTCATGATCGCTCTCTCTTGCGTCGTCTTTGCCGTGCTCCTCAGCGTGCCGATCTTCGAGGAGCGAGCGAAG
CAGAATTGCCTGGCGCTGCTGGGATTTGCCTCCATGCTGTGGTGCACGGAGGCCCTGCCACTCTACGTCACCTCCATGCTAGT
GCCCCTGCTGGCTGTTGTGCTCAGAGTGATGGTGGACGACAGCGGGAAGCACCCGGTCAGGAAGAGCGCACCCGACGCTGCGG
ACGCCATCTTCAAAGCCATGTTCTCACAGGCGAGTTCACAGCTCTTCATCTCCCCTCATTGCACAATTGAGAGGCACGTTGAC
GGCCTGCCCTCATACCCCACTACAATCATGCTGCTGCTGGGGGGATTCGCGATAGCTAGCGCCTTCACAAAGCACTTCATCGC
CAAGCGCGTGGCCGTCTGGGTGCTGGGGAAAGTCAGCGCCAAGCCGCACGCGGTGCTGATAGCCAACATGTTCGTGGCCACCT
TCGCATCCATGTGGATCACCAATGTGGCCGCCCCCGTGCTGTGCTTCTCCGTGCTGGACCCAATCCTGCGCACGCTGCCCTCC
GGCCATTCTTTTGGCAAAGCTCTGGTCCTGGGCATCGCGCTGGCGTCCAACCTGGGAGGCATGACAAGCCCGATCTCATCGCC
GCAGAACATCTTCGCGATCCAGGAGATGGGCCGAGATGGCGAGCCGCCCTCCTGGCTTGCCTGGTTTGCGGTGGCGCTGCCCG
TGGCGTGCGTGGGTAACTTTGCCTGCTGGGGATTTCTGCTGCTGGCCTACCGGCCCGGCCGCACCCTCAAGGAAGTCCGCCGT
ATGCCCTTCAGCTCGGACCCGTTCACGTGGAAGCAGATCTACGTGGTGGTGATCAGCCTGGGCACGGTGGGGCTGTGGTGCGC
CAACACCGCGCTCTCCAAGTTCACCGGCCAGATGGGCATCGTGGCCATCGTCCCCATGGTCGCCTTCTTTGGCTTCGGCCTCC
TCTCCAAGGATGACTTCAACAACCAGCTGTGGAACGTGGTGATGCTGGCGATGGGCGGCTCCGCGCTCGGCGAGGCCGTCAAG
TCCAGCGGACTTCTCTCCTCCATTGCGCACTCCATCGAGGACGTGGTTGCCGGCATGGGCGTCTGGGCCGTCTTCGCCATCTT
CTGCGCGCTTGTGCTCGTGGCCACAACCTTCATCTCCCACACCGTGGGCGCCATGGTCATCCTGCCCATTGTCAGCGCTGTTG
GCGCGCAAATGGAGGAGCCCCACCCGCGGCTGCTGGTGATGGGGGCAGCTTTGATGTGCAGCGGAGCCATGGGCCTGCCGGTG
TCAGGCTTCCCCAACATGACAGCCTACGCCAAGGAGGACCCCACCGGCAACCCCTGGCTATCCACCATCGACTTCTTCAAGGT
GGGCGTGCCATGCTCACTGGCCACGTATGGCCTCATCGTGACAGTAGGCTATGGCATCATGAAGTTCGTTCTGGGCTGGTGA
>Coccomyxa_subellipsoidea.PTC2/homologue
ATGAAGTTTGGCGCGGAGAGGGCAGGCCACGCGCTGCTGAGCTGGCTGACTGCTGCCTGGCTGTGGCTTCTGCAAGCCTGGGA
GGTGGTCGCAGAGTGGGGCCGCCAGTGCTGGGGGGCTCTTTTGCATGCCTGGCATTATATCGCCAGCGCTGTCATGCAGGCAG
TGCACTGGGTGAGGGGGCTCGAGGAGGTAGGAGGTGACCTGGACAGGACAATATCCCTGGCTCTTGAAGAGGGCGGGGACGAT
ATCAAGGGCGCATTTGACAGCGAGCTTAACCGCATTACTACTTTCCACAAAAAGAAGGAAGAGGAGCTTCTGGGTGCAGTGGA
CAAGCTTGGGGAGGAGGTGAGCAGTGCTGTGGAGCCAAGTGCACAGCAGAGCGCTCCTGATGCGAGCTCGCCGCTGCTTGGGA
CTTCCAGGAATGCGGAGGCACTGTACTGGGGCCAGGACACTGTTGCTGTGCGCATTGCGCGGGAGCAGCTAAGGGAGACCTTT
CAGGAGCTGTATGTGGAGATCCAGGGGCTGATAGATTTTGTGGAGGTGAATCGTACAGGCTTCAGGAAGGCCCTGAAGAAGCA
CGACAAGGTGTTGGGCGCGCTTGGGCACCCAAAGATGCAGCCAACGTACATGCCTAATGTCGAGGCTGCCTTCCCTGAGAAGA
ACCGCCTGCGTGTGTCAGAAGCTCAGAAGCAGCTGGTGGAGCTGTACGCTGTGGTGTGCTGCCACAACAACCTGCTGCTGGCC
CAGCTGGAGCTCAAGGCACAGCTGCGCTCCCAGCTCAAGCTTGAGAGGACGACAGTCTGGAAGGACATGGTGGAGAAGGAGCG
AAAGGAGAACGCTGCCACGGTCGACGACAGCGGCGCTGAGTCCAAGCCCTGGTACCGCAGCAGCCTCTTCATGATCGCTCTCT
CTTGCGTCGTCTTTGCCGTGCTCCTCAGCGTGCCGATCTTCGAGGAGCGAGCGAAGCAGAATTGCCTGGCGCTGCTGGGATTT
GCCTCCATGCTGTGGTGCACGGAGGCCCTGCCACTCTACGTCACCTCCATGCTAGTGCCCCTGCTGGCTGTTGTGCTCAGAGT
GATGGTGGACGACAGCGGGAAGCACCCGGTCAGGAAGAGCGCACCCGACGCTGCGGACGCCATCTTCAAAGCCATGTTCTCAC
AGGCGAGTTCACAGCTCTTCATCTCCCCTCATTGCACAATTGAGAGGCACGTTGACGGCCTGCCCTCATACCCCACTACAATC
ATGCTGCTGCTGGGGGGATTCGCGATAGCTAGCGCCTTCACAAAGCACTTCATCGCCAAGCGCGTGGCCGTCTGGGTGCTGGG
GAAAGTCAGCGCCAAGCCGCACGCGGTGCTGATAGCCAACATGTTCGTGGCCACCTTCGCATCCATGTGGATCACCAATGTGG
CCGCCCCCGTGCTGTGCTTCTCCGTGCTGGACCCAATCCTGCGCACGCTGCCCTCCGGCCATTCTTTTGGCAAAGCTCTGGTC
CTGGGCATCGCGCTGGCGTCCAACCTGGGAGGCATGACAAGCCCGATCTCATCGCCGCAGAACATCTTCGCGATCCAGGAGAT
GGGCCGAGATGGCGAGCCGCCCTCCTGGCTTGCCTGGTTTGCGGTGGCGCTGCCCGTGGCGTGCGTGGGTAACTTTGCCTGCT
GGGGATTTCTGCTGCTGGCCTACCGGCCCGGCCGCACCCTCAAGGAAGTCCGCCGTATGCCCTTCAGCTCGGACCCGTTCACG
TGGAAGCAGATCTACGTGGTGGTGATCAGCCTGGGCACGGTGGGGCTGTGGTGCGCCAACACCGCGCTCTCCAAGTTCACCGG
CCAGATGGGCATCGTGGCCATCGTCCCCATGGTCGCCTTCTTTGGCTTCGGCCTCCTCTCCAAGGATGACTTCAACAACCAGC
TGTGGAACGTGGTGATGCTGGCGATGGGCGGCTCCGCGCTCGGCGAGGCCGTCAAGTCCAGCGGACTTCTCTCCTCCATTGCG
CACTCCATCGAGGACGTGGTTGCCGGCATGGGCGTCTGGGCCGTCTTCGCCATCTTCTGCGCGCTTGTGCTCGTGGCCACAAC
CTTCATCTCCCACACCGTGGGCGCCATGGTCATCCTGCCCATTGTCAGCGCTGTTGGCGCGCAAATGGAGGAGCCCCACCCGC
GGCTGCTGGTGATGGGGGCAGCTTTGATGTGCAGCGGAGCCATGGGCCTGCCGGTGTCAGGCTTCCCCAACATGACAGCCTAC
GCCAAGGAGGACCCCACCGGCAACCCCTGGCTATCCACCATCGACTTCTTCAAGGTGGGCGTGCCATGCTCACTGGCCACGTA
TGGCCTCATCGTGACAGTAGGCTATGGCATCATGAAGTTCGTTCTGGGCTGGTGA
>Symbiochloris_reticulata.PTC1
ATGCAATTGGGCCTGGGCAGGGACGACATGCAGAGGCTGTTTGTCCTGCTGACGGGGCTGGAGCGTTACATCGATTTGAACAT
TGCCGGCTTCCGCAAGGCCCTGAAAAAGCACGACAAGGTTCTGGCAGATGCAGAGAGCGGCAAGCTGAAGGAGACCTACATGC
CCACTGTGCACCGCCAGTGCTGCCTCAACAAGAAGCCCATCCTGGAGACATTGTATGCCATCGTGTGCTGCGATGGGAACAAT
GAGATGGCTTTGATAGATCTCAAGCGCCGTCTCGGCGAGACTGTGCAATTTGAAAGAAACACAGTGTGGAAGGATATGGTGCA
AAAGGACCGCAAAAGGGGCACGCTGAAGGTCGACGATGGGCTGATCGGATCGTGGTGGCATCGCGCGCGGCAGCCGGCAGCAA
TCGCCATGTCACTGGCGGTCTTTGTTGTGCTTTTATATACGCCCACGTTCAGAGAGCCGGAGAAGCGAAACTGCCTGGCGCTA
CTGGCCTTCACCTCGCTGCTGTGGTGCACGGAGGCGCTGCCGCTGTACGTGACGAGCATGCTGGTGCCCCTGCTGGTGGTGGT
GCTGCGGGTGTTGGTGGACGGCAGCCAGCACCCGCCTCAGCGCCTGTCCTGCAAGCAGGCCGCGCCCCACATCTTCCATGCGA
TGAACTCCCAGGTGATCATGCTGCTGCTGGGAGGCTTCACCATTGCGGCTGCCCTGAGCAAGCACGCGATTGCCAAGATCCTG
GCCAGCTGGGTGCTGAGCAAGGTGGGGCAGCGGCCGGGCGCGGTGCTCATGGCCAACATGCTGGTGGCCACCTTTGCCAGCAT
GTGGATCTCCAATGTGGCCGCCCCCGTGCTGTGCTTCTCGCTTGTGCAGCCCGTTCTGCGCACGTTGGATGCCACCCACAGCT
TTGCAAAAAGCCTGGTCATGGGCATTGCGCTGGCATCCAATCTGGGGGGCATGACCAGCCCAATCAGCAGCCCACAAAACCTG
TTTGCCATTGAGCGCATGTCCATGGCAGGCCTTCCGCCCTCATGGCTGTCCTGGTTTGCAGTCGCACTGCCTGTGGCTTTTCT
GGGTAACTTTCTGGTCTGCGGCTTGTTGCTCCTTGTCTATCAGGACCCTCATTTCACCGAGGTCCGGCCAATGCAGCCCATCA
AGGATCCGATCAACGGCAAGCAGATGTACATCATTGCAGTATCTGTCGGCTCGGTCACAATGTGGTGCTTCAACAGCGTGCTC
CAGCAATGGTTTGGGGAGATGGGTATCATCGCTATACTGCCCATGATAGCATTTTACGGCTTTGGCATACTAGACAAGGACGA
TTTTAACAGCATGCTGTGGAATGTCGTGATGCTGGCTATGGGCGGGCTGGCGCTGGGGGAGGCGGTCACATCCTCTGGCCTGC
TGCTGTCCATTGCGGAGCAGCTGCAGCACCTGGTTCAGGGCGCCTCGGTGTGGCGCGTGCTGGTCATCTTCTGCGGCCTGGTG
CTCGTGGCCACCACCTTTGTCTCCCACACTGTCGGCGCCATGGTCGTCCTGCCCATCATTCAGTCTGTCGGCTCCCAGCTGTC
GGATCCCCATCCAAAGCTGCTGGTCATGGGCGCAGCATTGATGTGCTCAGGTGCCATGGGCCTGCCTGTCAGTGGCTTTCCAA
ACATGAATGCTGTGGCCTTGGAGGACTCCAAAGGCATCAACTATCTCACCACAATAGACTTCTTCAAGGTTGGCCTGCTGAGT
TCCTTGATAGCCTATGGACTTATCGTCACCCTGGGCTATGGCATCATGTACTATGGCATTGGCTGGTAA
>Edaphochlamys_debaryana.PTC1
ATGAAGTTCACTCACCAGCTCAAGTTCAATTCGGTTCCCGAGTGGCGGGAACATTACATACAGTATGCGCACCTTAAGAAATA
CATTTACGCGCTTGCGAAGAAGGAGGCTGACCACCAAGCGGACGGCGCCGGGACTGGTGATGTAGAGGGCCTGATCGCCCCAC
TGCTGCAGGATGGCGGTCGCGCATCGGGCCCCACCGAGGAGGGCTTCCAGCGCGAGCTGGACTCCCAGCTGGCCGCGCTGCTG
GGTTTCTTCGCGGTCAAGGAGGCGGACCTGCTGGCCAAGGTGTCGGAGCTGGAGCTGGAGGTGCAGAGCATGGAGAAGATCCC
CAACCGCAACGAGGCCTCCAACCTGGTCCGGGCGAGGGGGGGCGGCAGCGCCGCCAGCGGCACGCCCTCCCCGGGCGCCTCCC
CGCGCGCCTCCGCCGCTGGCGCCGCGCTGTCCGCCCTCAGCGGCCTGCTGGCGGCCTCGCCCTCCACCATGGACCTGGCGCGC
ATGGTGGCCGCCTCGCCGCCAGAGGACCACCGCTCCGTGCGCGTGGCCTTCTGGAAGAACCCCCCGCGTCACCTCTTCTCCTC
CAGCCTGCAGTCCCGCGCGGCCAAGCTGCAGAGCAGGTTCCAGGACCTGTACATTGCGCTCCACGACCTGCGCGAGTTCCTGC
ACATCAACAAGGAGGGCTTCCGCAAGATCATCAAGAAGCACGACAAGCTGACCCGCTCCGTGGACCTGCGCGCCCGCTGGTGG
CCCAACGTGGAGGCGCACCTGGCCCCCGCCGCCAAGCAGGCGGAGCTGGACGGGGCCATAGCGGGGCTCACGGACACGTACGC
GGTGGTGTACTGCCGCGGCGACGCCTCCTCCGCCGAGGAGCTGCTCAGCCGCGGCCTGCGCGAGCACATCACGGTGGAGCGCA
ACACCGTGTGGCGGGACATGGCGGCGCTGGAGCGCAAGTACGCGGCGGTCAGCGTCAAGCAGGCGGCGGGGGGGGCCAAGCCC
AGCTGGCTGTGGCGCCACGCCCGCTGGCTGAAGCTGGGCTTTGCGCTGGCGGTGTTTGGGATCATGCTGCAGTACGAGGTGTG
GCCCGGCCCCGAGAACGCCCCCCGCAACGGCTGCCTGGCGCTGCTGGTGTTCGCGTCGCTGCTGTGGTCGCTGGAGGCCGTGC
CCCTGTTCGTGACCTCCATGCTGCTGCCCCTGCTCATCGTGCTGCTGGGCGTGCTGGTGGACCGCACCAAGGACCCCCCGCAG
CGCATGACCCCGCAGCAGGCCGCACCCGCCATATTCCACGCCATGTTCTCGCAGACCATCATGCTGCTGCTGGGCGGCTTCGC
CATCGCCGCGGCGCTGTCCAAGCACGCAATCGCCAAGCAGTTCGCTGTGGCCATCCTGTCCCGCGTGGGCCGCCGCCCCCGCA
ACGTGCTCCTGGCCTCCATGTTCACCGCCACGTTCGCCAGCATGTGGATCAGCAACGTGGCGGCGCCGGTGCTGTGCTTCGGG
CTCATACAGCCCATCCTGCGCACGTTGGACCCCGGCCACCCCTTCGCCAAGGCGCTGGTGATGGGCATCGCCCTGGCCTCCAA
CGTGGGGGGCATGACCAGCCCCATCAGCAGCCCGCAGAACATCTTCGCCATTGAGCGCATGTCCCTGGACGGCCGCCCCCCCT
CCTGGCTGGCCTGGTTCGCGGTGGCGCTGCCCGTGTCCATAGCCTGCAACTTTGTGTGCTGGGGCCTGCTGCTGGCCGTGTAC
CGCCCCGAGCGGGTCATCGCCGAGGTGCGCCCCATCAAGCCCAACACGGACCCCATCAACGGAACGCAGGTGTACATCTGCGC
CGTGTCGCTGCTGACGGTGGGCGCCTGGTGCGCCAACACCTTCCTGCAGAAGTTCACGGGCGAGATGGGGGTGGTGGCGGTGG
TGCCGCTGGTGGCCTTCTTCGGCTTCGATGTGCTCAACAAGGACGACTTCAACTCCTTCCTGTGGAACGTGGTCATGCTGGCC
ATGGGCGGGCTGTGCCTGGGCGAGGCCGTCAAGAGCAGCGGGCTGCTGGCGGCGCTGGCGCTGGGCATCAGCGACCTGGTCAC
GGGGCTGAGCCTGTGGCAGGTGGCGGTGGTGTTCTGCGGAATGGTCCTGGTGGCCACCACCTTCATCTCGCACACCGTGGGCG
CCATGGTGATCCTGCCCATCGTGCAGTCCGTGGGCGAGGCCATGCCCGGCACGCCCCACCCCAAGCTGCTCGTCATGGCGGCG
GCGCTCATGTGCTCGGGTGCCATGGGCCTGCCCGTGTCCGGCTTCCCCAACATGAACGCCGTCAGCCTGGAGGACGCCACGGG
CAACGCCATCGTGGCCACGCAGGACTTCCTGTTGAGCGGTGTGCCCGGCAGCATTGCGGCGTACGGCATCATCGTGACGCTGG
GTCACCACACCATGGCACTGCTGGCAGCCCCCTGA
>Enallax_costatus.PTC1
ATGAAGTTCACGCATGTGCTGAAGTTCAACTCCGTGCCCGAGTGGCGGGAGTCGTACATCAACTACCCGCTGCTGAAGAAGCT
TATCCTTGCTGCAAGTACCGCTGAATATCATGAAGCGTACGAGGGCCTGGCGCTCACGCAAGATGAGGAGGCAGGGCCACGGT
CTCCTCTGCTGTCAGCCCAGCCCAGCCTCAGCCGTTCGTTATCCGTTACGATGACACGCGAGCAGCGCGAGAAGGAGTTCCTC
GAGGCCCTCGACAACGAGCTTGCGAAGATCATACGCTTCTATCTGAAGAAGGAGGCAGAGATCAGTGCCAAGTTTGAGGAGCT
CAGCATGATGGTCCATCATGCGGAGGGGATACCTTCTCCTACCCCAGAGCAAATGGCAGATGGCCACGACGTGACGACAGCAG
CGCGTGTAGCATTCTGGTCCCAGGGGGGCCGAGCAGTAGCAGCTCAGCGTGAAAAGCTTAAAACATCGCTCGAAGAGCTTTAT
GCCACAACGTTCAGCCTCGCTAACTATGTAGAACAGAACAGGACTGGCTTTCGCAAGATTCTCAAGAAACATGATAAGCTAGT
CTCGCACACAATGTCGAGCAACTATCTACCCATTGTAGACCAGAAGTTCCCAGCCAGCCACGCAGCCACCCTTCATCACCAGC
TAGAGGCAATCACAGCACTGTATGCAGTAGTATGCTGCAACGGTAACTTGGAGCATGCCAACAGCATACTACGTAAGCAGCAG
CAGGAGCAGGTGTCGTTCCAGCGTAACAGCATCTGGAAAGATATGGTGGGCCAGGAGCGACGAGCAGCAACAGTACGAGTGCA
GGACGGCAAAGAGGTAGAGCCAGAGTCCTGGTTCACAGCCCACCGGCAAGCAGTCATCCTGGCAATCGCACTAGCAGTGTTTG
TCGTGCTACTCACAGTACCCATATTCAAGCAGCCTGAGAAGCAGAATTGCTTGGCATTGCTGGCCTTTGCAAGTATGTTATGG
TGCACGGAGGCCATTCCGCTGTTTGTCACCAGCATGTTAGTGCCTTTCCTGGTGGTGGTGTTGCAAGTGCTTGATGATGTGAC
TCAAGAGCCCCCTGAGAGGCTGACACCCAAACAGGCGGCACCCAGGGTTTTCCACACCATGTTCTCACAGACCATCATGTTGC
TACTAGGAGGCTTTGCCATTGCAGCAGCCCTCAGTAAACACTTCATTGCCAAGCAGCTCGCAGTAGCAATACTCTCTAGAGTA
GGCCGCAAGCCACATCACGTACTCCTGGCCAATATGCTCGTGGCAACATTTGCCAGCATGTGGATATCCAATGTAGCAGCGCC
TGTTCTGTGTTTCAGCTTGGTACAGCCCATACTTCGAACCCTGCCGACTACGCATGCTTTCTGCAAGAGCTTGGTCATTGGGA
TTGCACTGGCGAGCAACTTAGGCGGAATGACGAGTCCTATCGCCAGCCCCCAGAATATATTTGCAGTCGAGAGGATGGGAATG
GGTGGTACACCACCTAGTTGGCTGGAGTGGTTTGCTATAGCTCTGCCTGTCAGCTTTCTGGGCAACCTGCTGTGCTGGGGGCT
GCTACTGTTGGTGTACAAGCCTGGGAAAGATATAAAGGAGGTTCGTCCTCTGAAGCCCACTGAGGATCCTTTGACTGGCACAC
AGATATATGTCATCGTCATCAGCTTGGCTACAGTCACACTGTGGTGCTGTAACAGCTTCCTACAGGAATATACCGGAGAGATG
GGTGTTCTCGCCATCTTCCCTCTGGTTGCGTTCTTTGGCTTTGGTGTTTTGAACAAGGATGACTTCAACGGCTTTCTTTGGAA
CGTTGTGATGCTGGCTATGGGTGGACTGGCACTGGGAGAAGCTGTGCAGAGCAGTGGGTTACTGCTGGAGATATCAAATAGCA
TCAGTCACCTTGTTGCTGGTCAGAGCCTGTGGGCAGTCCTTGCCATCTTCTGTGGACTAGTGCTTGTGGGCACAACATTCATC
AGCCACACAGTTGGAGCCATGGTGATCTTGCCCATTGTGCAAGCAGTGGGGCAGCAGATGCCGGGTGGAGATCATTCAAAGCT
GCTGGTTATGGGTGCAGCTCTTATGTGTTCAGGTGCCATGGGCTTACCCGTAAGTGGCTTCCCCAACATGAATGCCGTTGCAT
TAGAAGATCCCACTGGGGCAAACTATGTCTACACAAAGGACTTCCTATTGGTTGGGGTGCCTGGCAGTATCATGGCATATGGC
ATCATCATCAGCGTAGGGTATCTGCTGATGTTGGCAGTAGGCTTTTAG
>Mesostigma_viride.PTC1
ATGAAGTTCGGGAAGGTCCTGAAGGACGATGCCGTCCCTGATTGGATTCCGAAATACGTGGCATACAAGAAGTTGAAGCGTGT
CGTTCAACGGATGGAGTTAACAGTAGAGCAGGAACTGCAACAAGCCGCGAGCAAACGGGGAGCAGCAGGCTCGTCAGACGTAA
CCTCCCCTCTTGCCACCAAGGAGACGTTGCTGCAGAGGAAGAGTGATGAGTTTATGGAAGGGGTGGAGGAGGAGGTGGCCAAA
GTGAACCACTTTTACGACGAGATGGTCTCGGCGCTCCGCTGCGACCTGGAGGCCTACGAGAAGCAGCTCGCGGCGCAGCTCGC
GGGCGGCAACAAGAAGGCGTTCCAGAAGATGTTTGTGCTGGCGTCCGACCTGAATGCGTACATCACGCTCAACAGCACGGCGT
TCCGCAAGATCATGAAGAAGCACGACAAGTTGACGGGCCTGCACCGCATGGACGCGTTTGTGGCGCGCATCAAGCATGAGGGG
TTCATGGAGGCAAAGGCGCTGAGGGAGCTATCCGCACGCCTCGAGGCGATGATGTCGCCCGACGCGCTCGACAGCCTCAAGCA
GCAGTACCACCTGGAGCGCCAGAAGCGGTCCGAGTCCGCAGGGGGCTCCACCGGATCCCCAGCCAAGCCCACGCGCATCCTCT
TCTCCATCGCTGTCTTCTTCCTCATCCTGGCGCTGCCCCCCTTTTGGAGCGCGCGCCCGGCGAGCGGCGGCAACGATGACGGG
ATCGCTGACGTCAGCGACGGTGCCGGCGTCAGCGGTGGTGTTGCGTTTGGGGTTGATTATGGGTATGAGGGTGAGCCGGCGTC
GTTGGGCGCCCAGGGAGGCGTCGGGGAGGCCGCGGTGGCGGCGCGTGACCGGCTCATGCGTGTACTGTGGGAGCGGCACTATG
CGAGGGATGAGGCGGCCTCCTCGAGCATCGGCGACTACGTCTCTGGCAACAGTGCGTTTGGCCCCACTCAGGAGGAGCGCGCG
CACCGGTGCTTTGCGCTGCTCATCTTCATCGCGTGCATGTGGGTGCTGGAGGCGCTGCCGTACTTCGTCACCTCCCTCATGAT
CCCGCCCCTGGTGGTCATGCTGAACATCATGGCGGACCCGACGGACAAGGACAAGGCACTGTCCGCGCCCGACAGCTCGCGCC
TCGTCCTCTCGTCCATGTTCGACCACGTGCTCATCCTGCTGCTGGGCGGGTTCACGCTCTCCGCCGCGTTCGGGCAGTGCGCG
TTTGAGCTGCGCATCGCAGGCGCGCTGCAGCGGGCGCTCGGCCACCGCCCCTGGCTCTTCATGCTCGCCATCATGCTCCTCTC
GCTGTTCCTCTGCATGTGGCTGTCCAACGTGACCGCGCCCGTCCTCATGCTCTCGGTGCTGCTGCCCATCCTGCGCGACTTCG
ACCACGGCGGGCGGTACCCTAAGGCCCTGCTGCTCGGCCTCGCGTTTGCCTGTAACCTGGGCGGCATGGTCACCCCGATCGCG
TCGCCTCAGAACGCGGTCGCCCTGGTGGCGCTCGACGCGCAGCACTTTACCATCACCTTCTTCGAGTGGATGGCGGTCGCGCT
GCCCTTCTGCGTGCTCCTCGTCGTCGTCGTCTGGGCCTACCTCATCTTCGCGCTGCGGCCAGACGACGTGGTGTCCATCCCGC
CGGTCATGTACAAGACGACCCCCCTGAGCAGCAAGCACATCTGGGTGCTCCTCTTCTCGCTGGCCACCATCGGCCTGTGGTCC
ACCCTCTCCCTCACCGTGAGCGTGCTGGGGGACCTGGGCATCATCGCGCTTCTCTTCATGGTGTTTGCGTTCGGCACGGGCGT
GCTGTCCAAGCACGACCTGAACTCCTTCTCCTGGCACCTGCTGCTGCTGATCGCCGGTGGCAACGTCCTGGGCCGCGCGGTGC
AGTCCTCCGGGCTGATCCAGATCGTAGCGCAGATCGTGACGCCCTACCTGCACGACATCCTGTGGGTTGCGGCGCTCGAGCTG
CTCGCCTTCATGATCATCATCACCACGTTTGTCTCCCACTCGGTCGCGGCCATCATCATGATGCCTCTCATCGTGGCGATCGG
GAAGGAGATATCACCTCTCTCCGCCGAGGTGCTGGTCCTCCTGTGCACGCTCGCGGACAGTGCCGCGATGGCGCTCCCGATGA
CATCCTTCCCCAACGTGAACTCGCTGCTCGTGGAGGATGACTATGGCGTTCCCTACCTCCGAGTCGTCGACTTCATCAAAGTG
GGTGCCCCCGTGTCGATCATGGTGGTGACCGCCATCGCCACCCTGGGATACTCCCTGGCTGTGTTTGTGCTGCGCCCATGA
>Raphidocelis_subcapitata.PTC1
atgaAGTTCACCCACCAGCTCAAGTTCAACGCGGTGCCGGAGTGGAAGGAGCATTACATAAACTACCCCCTCCTGAAAAAGAT
CATCTACGCgacccgcgcggccgagtgCCAGGACGCGTacgacggcgtcggcggggacgaggaggcggccggcccctccgcct
ccggcggctcgctgctgcgctccccccgcaccagcctcagcggcggctcgctgcgcgcgccgctgctgcagggcgtgggcggg
ctgtcgctgtcgcggtcgggcagcgtcggcgcgcgcgcgggggactCTGAATTCATTAAGGCGCTGGACCAGGAGCTGGCCCG
CATCATAAGCTTCTACCTGCGGAAGGAGGGGGAGCTGACCTCGGCGTTCGAGTCCCTCAacctgcagctgcacagCCGCGATG
GCTgcgacgcggctgcgcccgccgcgggcggcgccggcggcggcggcggcggagccgcggggtttggcaccgcgccggcggcg
cccgcggcgggcgccgtggacggcgcggcggccgcagaggcgggggaggccgccgccgccgcggcggtcccgCAGTCGCaggc
ggagcggcagcggcgcgccgagttccagcggcgcaccgcctACTGGGCTGCCAACgaccgcggcgtggcggcggagcgggagc
ggTTCCGGCAGAAGCTGGTCGGGCTGTTTGTGCAGCTGGACGgcctCAAGAAGTACTTGGAGATGAACCACACCGGGTTCAGG
AAGATCCTCAAAAAGCACGACAAGGAGACCACgcagCACCAGTACAAGGACAGCTACATGGCCATAGTGGACGccaagctgcc
gctgcgcagcctcgAGGGGCTCAACCGCCTGATAGAGCGGCTCAGGGAGATGCACGCGGCGGTGTGCTGcaagggcAATCTGG
AAAAGGCGGAGCGGGAGCTGAGGAGCGAGTTGCgggaggagGTCGGTTTCGAGCGCAACACCGTTTGGCGCGACATGGTGGCC
ATGGAGCGGCGCACGGGGGCGgtggtgctgcaggAGCCCGCCCACGGCATCGCCGATGAGTCGCGCCAGGAGccgtggctgcg
ccgccactggcagccgctggcgctgtgcgTCTCGGGGCTCGCgtttgccgcgctgctggcggcgccgctgttcgagggcgcgc
cggagaAGCGGAACTGCCTCGCCATGCTCGCGTTTGTGAGCCTCCTGTGGTGcaccgaggcgctgccgctgtttgTCACGTCC
atgctggtgccgctgctggtggtggtgctgcgcgtgctggtggacagGACCGTGgagccgccggtgcggctgGAACCCCAGCA
GGCCGCACCCGCAATTTTCAGAGTCATGTTcgggcagGTCAtcatgctgctgctcggcggctttgccatcgccgcggcgctgt
cgaaGCATTTCATCGCAAAGCAGCTGGCGGTCGCCATCCTCTCCCGCgtgggacggcggccgcgggacgtgctgctggcgaAC
ATGCTGGTGGCCACGTTTGCGAGCATGTGGATATCAaacgtggcggcgccggtgctgtGCTTCAGCCTGGTGCAGCCCATcct
gcgcacgctgccgccgagccaCCCCTTTGCCAAGTCGCTCGTGATCGGCATCGCCCTCGCTTCGAAcctcggcggcatgACCT
CCCCCAtctcctccccccaaaacatcTTTGCCATCGAGCGCATGAGCATGGACGGCCACCCGCCCAGCTGGCTGGCCTGGttc
gcggtggcgctgccggtcGCGTTTGCGGGCAACGTGCTGTGCTGGGGGCTGATCCTGGCAGTCTACCGCCCTGGGCAGAAGAT
CCGGGAGgtccgcCCCCTGAAGCCCCCAGAGGACCCCCTGTCACCCACCCAGGTTTACGTCGTCGTCGTGTCACTCGCCACCG
TGGCGCTGTGGTGCTGCAACAGCCTGGTGGCGGGGGTGACGGGGGAGATGGGGGTGCTGGCCATCCTGCCGCTGGttGCGTTC
TTCGGCTTTGGCGTGCTGTCCAAGGACGACTTCAACGGCTTCCTGTGGAACGTGGTGATGCTGGCaatgggggggctggcgct
gggggaggcggtcaagagcagcgggctgctgctgacgattgcgcagTccgtcggccagcagctgcccggcccgccgcacgaCA
AGCTGCTCGtcatgggcgcggcgctcatgtgcagcggcgcgatggGGCTGCCTGTCTCGGGGTTTCCAAACATGAACGCCGTC
GCGCTGGAGGACCCAACGGGGGTGAATTACGTGGACACGATCGATTTTTTGAAGGTCGGCGTGCCGGGCAGCGTGCTCGCGTA
CTGGATCATAGTGACCGTGGGGTATGGGATCATGAGGGCCGTGGGGATGtga
>Symbiochloris_reticulata_Africa.PTC1
ATGAAGTTCACGAAGGAGTTGAAATATAACGCCGTGGAAGAGTGGCGCGCCCACTACATCAACTATGCCGCTTTCAAGCGGCT
CATATACGGCGAAGAGAAGCGCAAATTTGGCGATAACGAACGCATGGTGCCGGGAACGCCACAGGAAGATGACCATCCCACTC
AGGAGCCACTGCTACACCAGACAGATGACAAAGCTTTCATGAGCCTTTTGGACAGCGAACTGGCTCGTGTGCACGAATTTTAC
CTTGAAAGGGAGCGAGAGCTTGGTGGCCAGCTTGACAGCTTGCTGAGCCATGCGCGCACTGTGGAAGTCAATGAACGGCCTGC
CACCCCTTCAACAGAGCACGGCCGCAGATCTTCTGAGGGCAGATTACACCTTGCGAGGCGGAGCAGTTCCAGAATGCAGGGAG
CGCTGGCAGATTTGCAGGCAGAAGCCGTATCCTCAGAGTTCTGGTCCCAGAACCAGGACTTTGCTGTCCAGGCTGCACGCGAG
CAACTCAGGGACGACATGCAGAGGCTGTTTGTCCTGCTGACGGGGCTGGAGCGTTACATCGATTTGAACATTGCCGGCTTCCG
CAAGGCCCTGAAAAAGCACGACAAGGTTCTGGCAGATGCAGAGAGCGGCAAGCTGAAGGAGACCTACATGCCCACTGTGCACC
GCCAGTGCTGCCTCAACAAGAAGCCCATCCTGGAGGGGGCGCTGCGGAAGCTGCAGACATTGTATGCCATCGTGTGCTGCGAT
GGGAACAATGAGATGGCTTTGATAGATCTCAAGCGCCGTCTCGGCGAGACTGTGCAATTTGAAAGAAACACAGTGTGGAAGGA
TATGGTGCAAAAGGACCGCAAAAGGGGCACGCTGAAGGTCGACGATGGGCTGATCGGATCGTGGTGGCATCGCGCGCGGCAGC
CGGCAGCAATCGCCATGTCACTGGCGGTCTTTGTTGTGCTTTTATATACGCCCACGTTCAGAGAGCCGGAGAAGCGAAACTGC
CTGGCGCTACTGGCCTTCACCTCGCTGCTGTGGTGCACGGAGGCGCTGCCGCTGTACGTGACGAGCATGCTGGTGCCCCTGCT
GGTGGTGGTGCTGCGGGTGTTGGTGGACGGCAGCCAGCACCCGCCTCAGCGCCTGTCCTGCAAGCAGGCCGCGCCCCACATCT
TCCATGCGATGAACTCCCAGGTGATCATGCTGCTGCTGGGAGGCTTCACCATTGCGGCTGCCCTGAGCAAGCACGCGATTGCC
AAGATCCTGGCCAGCTGGGTGCTGAGCAAGGTGGGGCAGCGGCCGGGCGCGGTGCTCATGGCCAACATGCTGGTGGCCACCTT
TGCCAGCATGTGGATCTCCAATGTGGCCGCCCCCGTGCTGTGCTTCTCGCTTGTGCAGCCCGTTCTGCGCACGTTGGATGCCA
CCCACAGCTTTGCAAAAAGCCTGGTCATGGTCGCACTGCCTGTGGCTTTTCTGGGTAACTTTCTGGTCTGCGGCTTGTTGCTC
CTTGTCTATCAGGACCCTCATTTCACCGAGGTCCGGCCAATGCAGCCCATCAAGGATCCGATCAACGGCAAGCAGATGTACAT
CATTGCAGTATCTGTCGGCTCGGTCACAATGTGGTGCTTCAACAGCGTGCTCCAGCAATGGTTTGGGGAGATGGGTATCATCG
CTATACTGCCCATGATAGCATTTTACGGCTTTGGCATACTAGACAAGGACGATTTTAACAGCATGCTGTGGAATGTCGTGATG
CTGGCTATGGGCGGGCTGGCGCTGGGGGAGGCGGTCACATCCTCTGGCCTGCTGCTGTCCATTGCGGAGCAGCTGCAGCACCT
GGTTCAGGGCGCCTCGGTGTGGCGCGTGCTGGTCATCTTCTGCGGCCTGGTGCTCGTGGCCACCACCTTTGTCTCCCACACTG
TCGGCGCCATGGTCGTCCTGCCCATCATTCAGTCTGTCGGCTCCCAGCTGTCGGATCCCCATCCAAAGCTGCTGGTCATGGGC
GCAGCATTGATGTGCTCAGGTGCCATGGGCCTGCCTGTCAGTGGCTTTCCAAACATGAATGCTGTGGCCTTGGAGGACTCCAA
AGGCATCAACTATCTCACCACAATAGACTTCTTCAAGGTTGGCCTGCTGAGTTCCTTGATAGCCTATGGACTTATCGTCACCC
TGGGCTATGGCATCATGTACTATGGCATTGGCTGGTAA
>Tetradesmus_deserticola.PTC1
ATGAAGTTCACCCACACCCTCAAGTACAATTCCGTGCCTGAGTGGCGCGAGTCCTACATCAACTATAGCCTGCTGAAAAAGCT
TATCTTAGCGGCCAGTACTGCAGAATATCATGAGGCGTACGAAGGCGTGCATCCTGCAGCAGACCTGGAGGATGCTGGGCCCA
GGTCACCCCTGCTATCTAGGCAGGCAAGTCTGCAGGCAAGTCTTTCCAGGAGTCTCTCAGTCACGATGACGCGCGAGCAGCGC
GAAAAGGAGTTCCTTGAGACATTGGACAACGAGCTGGCCAAGATCATCCGCTTTTACTTGAAGAAGGAGGCAGAGATCACAGC
CAAGTATGAAGAAGTCAGCATGATGGTGCAGCATGCCGAGGGCATTGCATCGCCAACACCAGGGCAGGCAGCAGAAGTCTCGG
GGTTGCAGGCAGCACAGCGCACAGCGTTCTGGTCTCAGAGCAGCAGGCCAGTAGCAGCGCAGCGCGAAAAGCTGCGAGCCGCA
CTGGAGGACCTGTACGCGACCTGCTGCAACCTTGCCAGCTATGTAGAGCAGAACCGGACTGGCTTCAGGAAGATATTGAAGAA
GCATGACAAGCTGGTGTCGCACCCGATGTCAGCCATATACCTGCCCATCGTAGACCAGAAGTTCCCGGAAAGCCACGCAGCGC
ACCTGCGCGCACAGATGGACGCCATCGCGTCTCTGTACAGCATGGTGTGCTGCAACGGCAACGCAGACAAGGCGGCAGCCATC
CTGCGCAAGCAGCAGCAGGAGCAGGTGTTCTTTGAGCGCAACAGCATCTGGAAGGACATGGTGGGCCAGGAGCGGCGGGCTGC
CACGCTGCACCTGCAGGATGGCAAGGAGGCTGTGCAGGAGTCCTGGCTGAGCACGCACCGCCAGGCGATGCTGGTCACCCTCG
CACTGGCAGTGTTTGCCTTCTTACTCTACTACCCAATCTTCAAGGAGCCAGAGAAGCAGAACTGCTTAGCGCTGCTGGCATTT
GCCAGCATCCTGTGGTGCACGGAGGCCATCCCGCTGTTTGTGACCAGCATGCTGGTGCCCTTCCTCATCGTGCTGCTGCGGGT
GCTGGATGATGTGGACCAGGAGCCGCCAGCTCGCCTGACACCTCAGCAGGCGGCACCGCGCGTCTTCCACACCATGTTTTCGC
AGACTATCATGCTGCTGCTTGGCGGCTTTGCCATTGCAGCAGCGCTGTCTAAGCACTTTATCGCAAAGCAGCTGGCTGTGGCC
ATCCTGTCGCGTGTTGGCCGCAAGCCGCACCACGTGCTGTTGGCAAACATGCTCGTCGCCACCTTTGCAAGCATGTGGATCTC
AAACGTAGCAGCACCCGTGCTCTGCTTTAGCTTGGTGCAACCCATCTTGCGGACCCTGCCCACAAACCATGCGTTCTGCAAAA
GCCTTGTCCTCGGCATCGCACTTGCCAGCAACCTGGGTGGCATGACGAGCCCAATCAGCAGCCCGCAGAACATCTTTGCGATT
GAGCGCATGAGCATGGGTGGCAGCCCGCCCAGCTGGCTGCAGTGGTTTGCGATCGCGCTGCCTGTCAGCTTCCTTGGCAATGT
GCTGTGCTGGGCGGTCATCCTGGCGGTGTACAAGCCAGGGCAAAACATCAAGGAGGTGCGCCCGCTCAAGCCTAATGAGGACC
CCATGAGTGGCACGCAAATCTACACCATCATCGTCAGCTTGGCAACTGTCACAGCCTGGTGCTGCAACTCGTTCCTACAGGCG
TACACTGGTGAGATGGGTGTGCTGGCAATCATCCCGTTGGTGGCCTTTTTTGGCTTTGGTGTGCTGTCCAAGGATGACTTCAA
TGGCTTCCTGTGGAATGTGGTCATGCTGGCCATGGGAGGGCTGGCGTTGGGGGAGGCAGTGCAGAGCAGTGGACTGCTGGCAA
CCATCTCAAACTTGATAAGCGATCTTGTGGGTGGTCAGTCGCTGTGGGCAGTGCTTGCCATCTTCTGTGCCCTGGTGCTGGTC
GGCACAACCTTCATCAGCCACACCGTTGGGGCTATGGTCATACTGCCTATCGTGCAGTCAGTGGGAGATAAGATGCCTGGGGG
CCATTCCAAGCTGTTGGTGATGGGAGCAGCACTCATGTGCTCAGGTGCTATGGGCCTGCCAGTGAGTGGCTTCCCAAACATGA
ACGCGGTGTCGCTGGAGGACTCGACCGGCCAGAACTACATCGGCACGGCAGACTTCCTCAAGGTCGGCGTGCTGGGCAGCGTG
CTGGCATACGGCATCATCATCAGCATAGGCTACGGGCTCATGCTGGCGGTTGGCTTCTAG
>Tetraselmis_striata.PTC1
ATGAAGTTTGAGCACGCGCTCGAGTTCAACAGCGTGCCGGAATGGCGCGGGCACTACCTCAACTACGAGCAGCTCAAGCGCCT
GGTGTATGCCGTGGAGGCCCAGCAGAGCGCAGCGCAGCGCGCTAGCCTGGACCTGTCCCGGCGGCCCTCCGGGGTGCAAGAGG
ATCCGGAGGCCGGGTCGCCGCTACTGCCGGGCGGCTCGGAGGTGGAGGGCGGCCAGGAGGCGGAGGCGGAGTTTGTGAGCTGC
GCGGAGGGGGAGCTCAAGCGGGTGCACGCCTTCCTGACTGCACGGGAGGCGGGCCTGCTGGGGCAGTGGGAGGAGGCGGCGCT
TGCGGCCCACAGCGCGGAGGCCAGCTACGTGCCAGCGCGCACCACTCGCGGAGGGGCGTTCACGCGCTCCCACTGGTGGCAGC
AGCCAACGATGCAGGCGCAGCGGCGCACGCTGGTGGCCACCCTGGGCAGCCTCTTTGTGAGCCTGCACGACCTGTCCAGCTAC
GCGGAGCTCAACGAGACGGGTTTCCGCAAGATCCTGAAGAAGCACGACAAGGTGACGGGCGGCGCGCTCAAGGGGGCGCTGCT
GCCGGTGGTGCAGGCCCGGCTGGGCGCCAAGCGCGCGCGGCTGGATCAGGCGCTCGAGGAGGTGACGAGCCTCTACGCCACGC
TCGCCTTTGACGGCGATGCGGACGTCGCCGCGGCGCACCTGAGGGAGGGGCTGCGCGAGCAGGTTGTGTTTGAGCGCAGTGCG
GTGTGGAAGGACCGCATGGAGGAGGAGCGTCGGGTTGCGACCGCGCACGTCGTGGGCCCCAAGGCCGCCGCCGCCAAGCCGTG
GCTGCTGTCCGGCAAGGCGATTGCAGGCCTGGCGGCGCTGGCGCTGGCGGGCGCTGTGCTGGGCAGCAGCGCGTTTGGGGCCG
ACGACGCTGGGGCCACCAAGCGCGCATGCCTTGCCATCCTGCTGGCCAGCGCGGTGCTGTGGTGCACCGAGGCGGTGCCGCTC
TACGTGACCAGCATGGCGCTCATCTTTGCGGTCGTCACGCTGCGCGCAATGCTGGACGGCGACGGGGCGCGCCTGAGCGCGCC
CGACGCCATGAAGCGCGTGTTCTCCAAGATCTTCAGCCAGACGGTCATGCTGCTGCTGGGCGGCTTCACCATGGCGGCTGCGC
TCTCTAAGCACCTCATCGCCAAGCGGCTCGCCATTGGCGTGATGGCGCAGGTGGGGCGGCGCCCGGCCTCGGTGCTCCTGGCG
GCGATGGGCATCGCGCTGTTCAGCAGCATGTGGATCTCCAACGTCGCGGCGCCCGTCCTGTGCTTCAGCATCGTGGCGCCCAT
CCTGCGCACGCTGCCCACGGACGACCCGCTGGGCGCCGCCATGGTCATCGGCATCGCGATGGCCTCCAACATCGGCGGCATGA
CGTCACCCATCGCGAGCCCGCAGAACATCTTTGCCATCGAGCGCATGTCCATGGACGGACACCCGCCCAGCTGGCTCGCGTGG
TTTGCGGTCTCCATGCCGGTCTCCATCACCTGCCTGCTGCTGGTGTGGCGCCTCCTGCTCATCATCTACCCGATCGACAGGGA
TCAGGAGGTGCGCCCGCTGCGGCAGCTGGACGACCCCTTCACGCTGCACCACGCCTTCGTCATCGCGGTGTGCCTGGCCACGA
TGGGCCTCTGGTGCGCCAACACGTGGCTGCTGCACCTGCTGGGCGGCATGGGGGTGACGGCGCTCATCCCCATGGTGGCGTTC
TTTGGCTTCGGGACCCTCGGCAAGGACGACTTTGAGAGCTTCCCGTGGAGCGTGGTCATGCTTGCCATGGGCGGCATCATCCT
GGGCGACGCCGCCACCGAGAGCGGGCTGCTGGCCGCCATGACAGAGCAGATTGTGGGCGTCGTGGGCAGCCTCACCGTCTGCG
AGGTGCTCGTCATCTTCACCGGCGTCATCGCCGTCGTCACCAGCTTCATCTCGCACACCGTGGGCGCCATGGTCATCCTGCCC
GTGGTGCAGAGCATCGGCGCGGAGCTCGCCAAGAGCACCGGGGTGGACCACAGCAAGCTCCTGGTGATGGGCGGAGCGCTGAT
GTGCTCGGGCGGCATGGCGCTGCCCGTCAGCGGGTTCCCCAACATGTCCGCGTCGTCCATCCAGGACCCCACGGGACGGAACT
ACGTCCACGTGGGCGACTTCCTCAAGACCGGCATCCCCTCCACTGCCATCACCTGGCTGTGCGTCATCGCCATCGGCTACCCC
ATCATGTCAGCCATCAACCTCTGA
>Trebouxia_sp..PTC1
ATGAAGTTTTCGCAGGCCTTGAAGGCCAATAGCGTTCCGGACTGGAAGCATCACTACATTCACTACTCACGCCTAAAGAAAAT
GATATTTCGACTGGAGCAGCTGCAAGGCAACGCCCCTCTGAGTCCTGTGCCTGAGCATAGGCAATCCTTGGATTTCACCAATC
CTTCAGCGCCCCTGCTGTCCAGACAGAGCTCTTCCATGCTGCAAAGGACCAGTTCAGGCCTTGAGCACGCTCATATCGACGAG
CTGATGTTTGAACGGGAAATTCACGATGAGCTAGCAAGAGTCAAAGCATTTTATGTTGAAAAGCATGATGAACTGGACGCAGA
GGTGTTGGCAGTCTTGGCAAAGGTTGCAGCAGCAGAGAGACGGGGCATCTCTGGTCCCGGTCATCAGGATGTTGAGGGCGGTC
AGTCTTTGCCAGAGGAGCAGCGAATAGCGTTCTGGACTGATGTGAATGTGCCTAGGAACATCAAGGAGCGCCTCAGTGGGGCC
CTGACAGACGTGTACATCCAGCTTGACAATCTATCCAAGTTTGTTGAGCTGAACTATGATGGATTCAGGAAGATCCTGAAGAA
GCATGACAAAATGACCAACACAGAGCTGTCAGGGCGGCTCATGCCCACAGTCTCAGACATGCTGGCCAAGGAGCAACGCAAAG
GGGCTCTGGAGGGCTTGAAGAACAGCGTGGTGCATGAGTACGCCCTCATAGCACACAGCGGCGGCGAGCGTGAGGCCGAGCAA
GAGCTGGGGCGGCACCGGCGGGATCAGCTTGACTTTTGA

1. A recombinant microalgal strain comprising in its genome a first modification which causes overexpression of a PSR1 gene, and optionally a further modification which reduces or eliminates expression from an endogenous PTC1 gene.

2. The recombinant strain of claim 1 comprising the further modification which reduces or eliminates expression from an endogenous PTC1 gene.

3. The recombinant strain of any one of claims 1 to 2 wherein the microalgal strain is a chlorophyte.

4. The recombinant strain of claim 3 wherein the chlorophyte is Chlamydomonas.

5. The recombinant strain of claim 3 or claim 4 wherein the strain is selected from the strains shown in Table 1.

6. The recombinant strain of any one of claims 1 to 5 wherein the PSR1 gene is
(i) from a species shown in Table 1 and/or
(ii) comprises any of SEQ ID No 2, or any of SEQ ID Nos 48 to 70, or 72 to 90 or a homologue or derivative thereof,
(iii) encodes any of SEQ ID No 1, or any of SEQ ID Nos 5 to 27, or 29 to 47 or a homologue or derivative thereof.

7. The recombinant strain of any one of claims 1 to 6 wherein the PSR1 gene has at least 75, 80, 85, 90, 95, 96, 97, 98, 99% or 100% identity with any of SEQ ID No 2, or any of SEQ ID Nos 48 to 70, or 72 to 90.

8. The recombinant strain of any one of claims 1 to 6 wherein the PSR1 gene encodes a PSR1 polypeptide having at least 75, 80, 85, 90, 95, 96, 97, 98, 99% or 100% identity with any of SEQ ID No 1, or any of SEQ ID Nos 5 to 27, or 29 to 47.

9. The recombinant strain of any one of claims 1 to 6 wherein the PSR1 gene encodes a homologue of a PSR1 polypeptide as shown in SEQ ID No 71.

10. The recombinant strain of any one of claims 1 to 9 wherein the first modification causes up regulation of an endogenous PSR1 gene.

11. The recombinant strain of any one of claims 1 to 9 wherein the first modification is expression of a PSR1 transgene.

12. The recombinant strain of any one of claims 1 to 11 wherein the PTC1 gene comprises the sequence as shown in SEQ ID 4, or any of SEQ ID Nos 134 to 165 or 167 to 176 or is a homologue or genomic equivalent of any of those sequences.

13. The recombinant strain of any one of claims 1 to 12 wherein the PTC1 gene encodes a PTC1 polypeptide having at least 75, 80, 85, 90, 95, 96, 97, 98, 99% or 100% identity with any of SEQ ID No 3, or any of SEQ ID Nos 91 to 123 or 125 to 133 or is a homologue thereof.

14. The recombinant strain of any one of claims 1 to 13 wherein the PTC1 gene encodes a homologue of a PTC1 polypeptide as shown in SEQ ID No 124.

15. The recombinant strain of any one of claims 1 to 14 wherein the further modification down-regulates or inactivates the PTC1 gene.

16. The recombinant strain of any one of claims 1 to 15, which strain:
(i) demonstrates at least a 10, 20, 30, 40, 50, 60, 70, 80, 90, 100, 200% increase in total phosphate or polyphosphate in the strain after culture for 60 hours under comparable conditions compared to a parent strain; or
(ii) demonstrates at least a 10, 20, 30, 40, 50, 60, 70, 80, 90, 100, 200% increase in phosphate removal efficiency by the strain after culture for 60 hours under comparable conditions compared to a parent strain; or
(iii) demonstrates at least a 10, 20, 30, 40, 50, 60, 70, 80, 90, 100, 200% decrease in complete-removing time of total phosphate in a medium after culture under comparable conditions compared to a parent strain; or
(iv) demonstrates at least a 10, 20, 30, 40, 50, 60, 70, 80, 90, 100, 200% decrease in total phosphate in a medium after culture for 60 hours under comparable conditions compared to a parent strain.

17. A biologically pure culture of a recombinant strain of any one of claims 1 to 16.

18. A cell extract; a cell suspension; a cell homogenate; a cell lysate; or a cell pellet of a recombinant strain of any one of claims 1 to 16.

19. A process for producing a recombinant microalgal strain having enhanced PRE efficiency of any one of claims 1 to 16, the process comprising the step of introducing a genetic modification into a parent strain which causes overexpression of a PSR1 gene.

20. The process of claim 19 further comprising the step of introducing a genetic modification into a parent strain which reduces or eliminates overexpression from an endogenous PTC1 gene.

21. A recombinant microalgal strain obtained or obtainable by the process of any one of claims 19 to 20.

22. A method of reducing inorganic or organic phosphorus in an environment, the method comprising introducing or culturing the recombinant strain as defined in any one of claims 1 to 16, or claim 21, into the environment.

23. The method according to claim 22 wherein the environment is a water body, optionally a waste water source from a municipal or aquacultural or agricultural source from which phosphorus is to be extracted.

24. The method according to any one of claims 22 to 23, which comprises a batch process by which the recombinant strain is added to the environment periodically over a period of time, and is optionally suspended in the environment.

25. The method according to any one of claims 22 to 23, which comprises a continuous flow process in which the recombinant strain is immobilised and exposed to an aqueous source from which phosphorus is to be extracted.

26. The method according to any one of claims 22 to 23, wherein the recombinant strain is exposed to the aqueous source from which phosphorus is to be extracted by raceway ponds, tubular photobioreactors (PBRs), flat panel PBRs, or soft frame PBRs.

27. The method according to any one of claims 22 to 23, wherein the recombinant strain is exposed to the aqueous source from which phosphorus is to be extracted via a permeable floating PBR.

28. The method according to any one of claims 22 to 23, wherein the recombinant strain is exposed to the aqueous source from which phosphorus is to be extracted in the form of a microalgal biofilm.

29. The method according to any one of claims 23 to 28 which further comprises the step of recovering the recombinant strain from the environment or reactor, optionally for use a fertiliser.

30. The method according to claim 29 which further comprises the step of heat-treating the recovered recombinant strain.

31. A fertiliser product obtained or obtainable from the method of claim 29 or claim 30, the fertiliser comprising, consisting or consisting essentially of the recombinant strain.

32. The fertiliser product of claim 30 which is slow release fertiliser or is liquid fertiliser.

33. A method of increasing the phosphorus availability in an environment, which is optionally a plant growing environment, the method comprising dispersing the fertiliser product of any one of claims 30 to 31 into the environment.

34. The method of claim 33 wherein the dispersion is by side-dressing into a growing crop.
